(19) United States
(12) Patent Application Publication
(10) Pub. No.: US 2022/0380770 A1
(43) Pub. Date: Dec. 1, 2022

(54) SHORT INTERFERING NUCLEIC ACID (siNA) MOLECULES AND USES THEREOF FOR CORONAVIRUS DISEASES

(71) Applicant: ALIGOS THERAPEUTICS, INC., South San Francisco, CA (US)

(72) Inventors: Leonid BEIGELMAN, San Mateo, CA (US); Antitsa STOYCHEVA, South San Francisco, CA (US); Aneerban BHATTACHARYA, South San Francisco, CA (US); David Bernard SMITH, South San Francisco, CA (US); Rajendra K. PANDEY, Foster City, CA (US); Saul Martinez MONTERO, San Bruno, CA (US); Vivek Kumar RAJWANSHI, Cupertino, CA (US); Jin HONG, Pacifica, CA (US)

(73) Assignee: ALIGOS THERAPEUTICS, INC., South San Francisco, CA (US)

(21) Appl. No.: 17/226,686

(22) Filed: Apr. 9, 2021

Related U.S. Application Data
(60) Provisional application No. 63/008,273, filed on Apr. 10, 2020.

(51) Int. Cl. C12N 15/113; A61K 31/713; A61P 31/14

(52) U.S. Cl. CPC C12N 15/1131; A61K 31/713; A61P 31/14; C12N 2310/14; C12N 2310/321; C12N 2310/346; C12N 2310/315; C12N 2310/313; C12N 2310/317; C12N 2310/345; C12N 2310/322

(57) ABSTRACT

The present invention is in the field of pharmaceutical compounds and preparations and method of their use in the treatment of disease. Described are short interfering nucleic acid (siNA) molecules comprising modified nucleotides, compositions containing the same, and uses thereof for treating or preventing coronavirus infections. In particular, the present invention is in the field of siNA molecules effective against a broad spectrum of coronaviruses, and especially the β-coronaviruses, including SARS-CoV-2, the causative agent of COVID-19.

### CROSS-REFERENCE STATEMENT

This application claims priority to U.S. Provisional Application No. 63/008,273, filed Apr. 10, 2020, the disclosure of which is hereby incorporated by reference in its entirety.

### FIELD OF THE INVENTION

The present invention is in the field of pharmaceutical compounds and preparations and method of their use in the treatment of disease. Described are short interfering nucleic acid (siNA) molecules comprising modified nucleotides, compositions containing the same, and uses thereof for treating or preventing coronavirus infections. In particular, the present invention is in the field of siNA molecules effective against a broad spectrum of coronaviruses, and especially the β-coronaviruses, including SARS-CoV-2, the causative agent of COVID-19.

### BACKGROUND

The following discussion is merely provided to aid the reader in understanding the disclosure and is not admitted to describe or constitute prior art thereto.

Coronavirus disease 2019 (COVID-19) (also referred to as novel coronavirus pneumonia or 2019-nCoV acute respiratory disease) is an infectious disease caused by the virus severe respiratory syndrome coronavirus 2 (SARS-CoV-2) (also referred to as novel coronavirus 2019, or 2019-nCoV). The disease was first identified in December 2019 and spread globally, causing a pandemic. Symptoms of COVID-19 include fever, cough, shortness of breath, fatigue, headache, loss of smell, nasal congestion, sore throat, coughing up sputum, pain in muscles or joints, chills, nausea, vomiting, and diarrhea. In severe cases, symptoms can include difficulty waking, confusion, blueish face or lips, coughing up blood, decreased white blood cell count, and kidney failure. Complications can include pneumonia, viral sepsis, acute respiratory distress syndrome, and kidney failure.

COVID-19 is especially threatening to public health. The virus is highly contagious, and studies currently indicate that it can be spread by asymptomatic carriers or by those who are pre-symptomatic. Likewise, the early stage of the disease is slow-progressing enough that carriers do not often realize they are infected, leading them to expose numerous others to the virus. The combination of COVID-19's ease of transmission, its high rate of hospitalization of victims, and its death rate make the virus a substantial public health risk, especially for countries without a healthcare system equipped to provide supportive care to pandemic-level numbers of patients. There is not yet a vaccine or specific antiviral treatment for COVID-19 and accordingly, there is a pressing need for treatments or cures.

SARS-CoV-2 is not the only coronavirus that causes disease. It is a β-coronavirus, a genus of coronaviruses that includes other human pathogens, including SARS-CoV (the causative agent of SARS), MERS-CoV (the causative agent of MERS), and HCoV-OC43 (a causative agent of the common cold). The infectivity of these viruses, and the severity of the diseases they cause, varies widely. B-coronaviruses can also manifest as zoonotic infections, spread to and from humans and animals. Additionally, non-human species such as camels, bats, tigers, non-human primates, and rabbits can be susceptible to β-coronaviruses. Accordingly, there is a pressing need for treatments or cures to multiple coronaviruses.

RNA interference (RNAi) is a biological response to double-stranded RNA that mediates resistance to both endogenous parasitic and exogenous pathogenic nucleic acids, and regulates the expression of protein-coding genes. The short interfering nucleic acids (siNA), such as siRNA, have been developed for RNAi therapy to treat a variety of diseases. For instance, RNAi therapy has been proposed for the treatment of metabolic diseases, neurodegenerative diseases, cancer, and pathogenic infections (See e.g., Rondindone, Biotechniques, 2018, 40(4S), doi.org/10.2144/000112163, Boudreau and Davidson, Curr Top Dev Biol, 2006, 75:73-92, Chalbatani et al., Int J Nanomedicine, 2019, 14:3111-3128, Arbuthnot, Drug News Perspect, 2010, 23(6):341-50, and Chernikov et. al., Front. Pharmacol., 2019, doi.org/10.3389/fphar.2019.00444, each of which are incorporated by reference in their entirety).

The present disclosure provides siNA molecules useful against coronaviruses, and especially SARS-CoV-2, the causative agent of COVID-19. Accordingly, the present disclosure fulfills the need in the art for compounds that can be safely and effectively treat or prevent coronavirus infections in humans.

### SUMMARY OF THE INVENTION

Disclosed herein are short interfering nucleic acid (siNA) molecules, which can be used to treat and/or prevent viral disease and infections, such as diseases (e.g., COVID-19) or infections caused by coronavirus like SARS-CoV-2. In some embodiments, the siNA can be a double-stranded siNA (ds-siNA).

In one aspect, the present disclosure provides siNA that comprise (a) a sense strand comprising a first nucleotide sequence, wherein the first nucleotide sequence is 15 to 30 nucleotides in length and comprises a nucleotide sequence that is at least about 60%, 65%, 70%, 75%, 80%, 85%, 90%, 95%, or 100% identical to an RNA corresponding to of any one of SEQ ID NOs: 1-1203 and 2411-3392; and (b) an antisense strand comprising a second nucleotide sequence, wherein the second nucleotide sequence is 15 to 30 nucleotides in length and comprises a nucleotide sequence that is at least about 60%, 65%, 70%, 75%, 80%, 85%, 90%, 95%, or 100% complementary to the first nucleotide sequence.

In another aspect, the present disclosure provides siNA that comprise a sense strand comprises (a) a first nucleotide sequence, wherein the first nucleotide sequence is identical to an RNA corresponding to 15 to 30 nucleotides within positions 190-216, 233-279, 288-324, 455-477, 626-651, 704-723, 3352-3378, 5384-5403, 6406-6483, 7532-7551, 9588-9606, 10484-10509, 11609-11630, 11834-11853, 12023-12045, 12212-12234, 12401-12420, 12839-12867, 12885-12924, 12966-12990, 13151-13176, 13363-13386, 13388-13416, 13458-13416, 13458-13520, 13762-13790, 14290-14312, 14404-14429, 14500-14531, 14623-14642, 14650-14687, 14698-14717, 14722-14748, 14750-14777, 14821-14846, 14854-14873, 14875-14903, 14962-14990, 14992-15020, 15055-15140, 15172-15200, 15310-15332, 15346-15367, 15496-15518, 15622-15644, 15838-15869, 15886-15905, 15985-16010, 16057-16079, 16186-16205, 16430-16448, 16822-16865, 16954-16976, 17008-17042, 17080-17111, 17137-17156, 17269-17289, 17530-17549, 17563-17582, 17680-17699, 17746-17765, 17857-17876, 17956-17975, 18100-18122, 18196-18218, 19618-19639, 19783-19802, 19831-19850, 20107-20130, 20776-20795, 21502-21524, 24302-24325, 24446-24465, 24620-24651, 24662-24684, 25034-25057, 25104-25128, 25364-25387, 25502-25530, 26191-26227, 26232-26267, 26269-26330, 26332-26394, 26450-26481, 26574-26600, 27003-27064, 27093-27111, 27183-27212, 27382-27407, 27511-27533, 27771-27818, 28270-28296, 28397-28434, 28513-28546, 28673-28692, 28706-28726, 28744-28794, 28799-28827, 28946-28972, 28976-29034, 29144-29172, 29174-29196, 29228-29259, 29285-29305, 29342-29394, 29444-29463, 29543-29566, 29598-29630, 29652-29687, 29689-29731, 29733-29757, or 29770-29828 of SEQ ID NO: 2407 and (b) an antisense strand.

In another aspect, the present disclosure provides siNA that comprise an antisense strand comprising (a) a second nucleotide sequence, wherein the second nucleotide sequence is complementary to an RNA corresponding to 15 to 30 nucleotides within positions 190-216, 233-279, 288-324, 455-477, 626-651, 704-723, 3352-3378, 5384-5403, 6406-6483, 7532-7551, 9588-9606, 10484-10509, 11609-11630, 11834-11853, 12023-12045, 12212-12234, 12401-12420, 12839-12867, 12885-12924, 12966-12990, 13151-13176, 13363-13386, 13388-13416, 13458-13416, 13458-13520, 13762-13790, 14290-14312, 14404-14429, 14500-14531, 14623-14642, 14650-14687, 14698-14717, 14722-14748, 14750-14777, 14821-14846, 14854-14873, 14875-14903, 14962-14990, 14992-15020, 15055-15140, 15172-15200, 15310-15332, 15346-15367, 15496-15518, 15622-15644, 15838-15869, 15886-15905, 15985-16010, 16057-16079, 16186-16205, 16430-16448, 16822-16865, 16954-16976, 17008-17042, 17080-17111, 17137-17156, 17269-17289, 17530-17549, 17563-17582, 17680-17699, 17746-17765, 17857-17876, 17956-17975, 18100-18122, 18196-18218, 19618-19639, 19783-19802, 19831-19850, 20107-20130, 20776-20795, 21502-21524, 24302-24325, 24446-24465, 24620-24651, 24662-24684, 25034-25057, 25104-25128, 25364-25387, 25502-25530, 26191-26227, 26232-26267, 26269-26330, 26332-26394, 26450-26481, 26574-26600, 27003-27064, 27093-27111, 27183-27212, 27382-27407, 27511-27533, 27771-27818, 28270-28296, 28397-28434, 28513-28546, 28673-28692, 28706-28726, 28744-28794, 28799-28827, 28946-28972, 28976-29034, 29144-29172, 29174-29196, 29228-29259, 29285-29305, 29342-29394, 29444-29463, 29543-29566, 29598-29630, 29652-29687, 29689-29731, 29733-29757, or 29770-29828 of SEQ ID NO: 2407 and (b) a sense strand.

In another aspect, the present disclosure provides siNA that comprise (a) a sense strand comprising a nucleotide sequence identical to an RNA corresponding to any one of SEQ ID NOs: 1-1203 and 2411-3392 and (b) an antisense strand.

In another aspect, the present disclosure provides siNA that comprise (a) an antisense strand comprising a nucleotide sequence identical to an RNA corresponding to any one of SEQ ID NOs: 1204-2406 and 3393-4374 and (b) a sense strand.

In some embodiments, the sense strand can comprise 15 or more modified nucleotides independently selected from a 2′-O-methyl nucleotide and a 2′-fluoro nucleotide, wherein at least one modified nucleotide is a 2′-O-methyl nucleotide and the nucleotide at position 3, 5, 7, 8, 9, 10, 11, 12, 14, 17, and/or 19 from the 5′ end is a 2′-fluoro nucleotide; and the antisense strand can comprise 15 or more modified nucleotides independently selected from a 2′-O-methyl nucleotide and a 2′-fluoro nucleotide, wherein at least one modified nucleotide is a 2′-O-methyl nucleotide and at least one modified nucleotide is a 2′-fluoro nucleotide.

In some embodiments, the sense strand can comprise 15 or more modified nucleotides independently selected from a 2′-O-methyl nucleotide and a 2′-fluoro nucleotide, wherein at least one modified nucleotide is a 2′-O-methyl nucleotide and at least one modified nucleotide is a 2′-fluoro nucleotide; and the antisense strand can comprise 15 or more modified nucleotides independently selected from a 2′-O-methyl nucleotide and a 2′-fluoro nucleotide, wherein at least one modified nucleotide is a 2′-O-methyl nucleotide and the nucleotide at position 2, 5, 6, 8, 10, 14, 16, 17, and/or 18 from the 5′ end is a 2′-fluoro nucleotide.

In some embodiments, the sense strand can comprise 16, 17, 18, 19, 20, 21, 22, 23, or more modified nucleotides independently selected from a 2′-O-methyl nucleotide and a 2′-fluoro nucleotide. In some embodiments, 70%, 75%, 80%, 85%, 90%, 95% or 100% of the nucleotides in the sense strand can be modified nucleotides independently selected from a 2′-O-methyl nucleotide and a 2′-fluoro nucleotide.

In some embodiments, (i) at least 2, 3, 4, 5, or 6 modified nucleotides of the sense strand are 2′-fluoro nucleotides; (ii) no more than 10, 9, 8, 7, 6, 5, 4, 3, or 2 modified nucleotides of the sense strand are 2′-fluoro nucleotides; (iii) at least 10, 11, 12, 13, 14, 15, 16, 17, 18, 19, 20, 21, or 22 modified nucleotides of the sense strand sequence are 2′-O-methyl nucleotides; and/or (iv) no more than 25, 24, 23, 22, 21, 20, 19, 18, 17, 16, 15, 14, 13, 12, 11, 10, 9, 8, 7, 6, 5, 4, 3, or 2 modified nucleotides of the sense strand are 2′-O-methyl nucleotides.

In some embodiments, the antisense strand can comprise 16, 17, 18, 19, 20, 21, 22, 23, or more modified nucleotides independently selected from a 2′-O-methyl nucleotide and a 2′-fluoro nucleotide. In some embodiments, 70%, 75%, 80%, 85%, 90%, 95% or 100% of the nucleotides in the antisense strand are modified nucleotides independently selected from a 2′-O-methyl nucleotide and a 2′-fluoro nucleotide.

In some embodiments, (i) at least 2, 3, 4, 5, or 6 modified nucleotides of the antisense strand are 2′-fluoro nucleotides; (ii) no more than 10, 9, 8, 7, 6, 5, 4, 3, or 2 modified nucleotides of the antisense strand are 2′-fluoro nucleotides; (iii) at least 10, 11, 12, 13, 14, 15, 16, 17, 18, 19, 20, 21, or 22 modified nucleotides of the antisense strand sequence are 2′-O-methyl nucleotides; and/or (iv) no more than 25, 24, 23, 22, 21, 20, 19, 18, 17, 16, 15, 14, 13, 12, 11, 10, 9, 8, 7, 6, 5, 4, 3, or 2 modified nucleotides of the antisense strand are 2′-O-methyl nucleotides.

In some embodiments, the sense strand and/or the antisense strand can comprise one or more phosphorothioate internucleoside linkage(s). In some embodiments, the siNA can further comprise a phosphorylation blocker and/or a 5′-stabilized end cap.

In some embodiments, the sense strand can further comprise a TT sequence adjacent to the first nucleotide sequence.

In some embodiments, at least one end of the siNA can be a blunt end. In some embodiments, at least one end of the siNA can comprise an overhang, wherein the overhang comprises at least one nucleotide. In some embodiments, both ends of the siNA can comprise an overhang, wherein the overhang comprises at least one nucleotide.

In some embodiments, the sense strand can further comprise at least 1, 2, 3, 4, 5, 6, 7, 8, 9, 10, 11, 12, 13, 14, 15 or more phosphorothioate internucleotide linkages. In some embodiments, the antisense strand can further comprise at least 1, 2, 3, 4, 5, 6, 7, 8, 9, 10, 11, 12, 13, 14, 15 or more phosphorothioate internucleotide linkages.

In some embodiments, the sense strand and/or the antisense strand can comprise one or more modified nucleotides. In some embodiments, the modified nucleotides are independently selected from 2′-O-methyl nucleotides and 2′-fluoro nucleotides. In some embodiments, at least one 2′-fluoro nucleotide or 2′-O-methyl nucleotide is a 2′-fluoro or 2′-O-methyl nucleotide mimic of Formula (V):

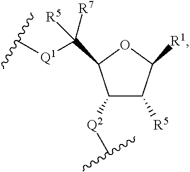

wherein R¹ is a nucleobase, aryl, heteroaryl, or H, Q¹ and Q² are independently S or O, R⁵ is —OCD₃, —F, or —OCH₃, and R⁶ and R⁷ are independently H or D.

In some embodiments, the sense strand and/or antisense strand comprises at least one modified nucleotide selected from

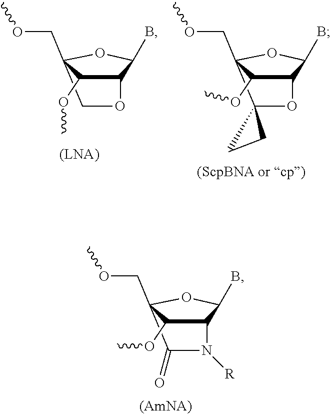

where R is H or alkyl (or AmNA(N-Me)) when R is alkyl);

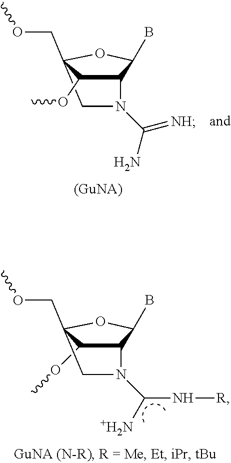

wherein B is a nucleobase.

In some embodiments, the siNA can further comprise a phosphorylation blocker and/or a 5′-stabilized end cap. In some embodiments, the phosphorylation blocker has the structure of Formula (IV):

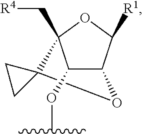

wherein R¹ is a nucleobase, R⁴ is —O—R³⁰ or —NR³¹R³², R³⁰ is C₁-C₈ substituted or unsubstituted alkyl; and
R³¹ and R³² together with the nitrogen to which they are attached form a substituted or unsubstituted heterocyclic ring. In some embodiments, R⁴ is —OCH₃ or —N(CH₂CH₂)₂O. In some embodiments, the phosphorylation blocker is attached to the 5′ end of the sense strand. In some embodiments, the phosphorylation blocker is attached to the 5′ end of the sense strand via one or more linkers independently selected from a phosphodiester linker, phosphorothioate linker, and phosphorodithioate linker.

In some embodiments, the 5′-stabilized end cap is a 5′ vinylphosphonate. In some embodiments, the 5′ vinylphosphonate is selected from a 5′-(E)-vinyl phosphonate or 5′-(Z)-vinyl phosphonate. In some embodiments, the 5′-vinylphosphonate is a deuterated vinyl phosphonate. In some embodiments, the deuterated vinylphosphonate is a mono-deuterated vinylphosphonate or a di-deuterated vinylphosphonate

In some embodiments, the 5′-stabilized end cap has the structure of Formula (Ia):

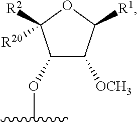

wherein R¹ is a nucleobase, aryl, heteroaryl, or H; R² is

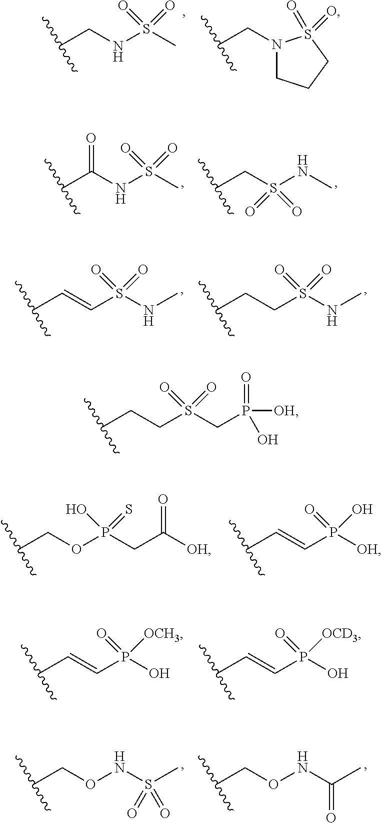

—CH═CD-Z, —CD=CH—Z, —CD=CD-Z, —(CR²¹R²²)ₙ—Z or —(C₂-C₆ alkenylene)-Z and R²⁰ is hydrogen, or R² and R²⁰ together form a 3- to 7-membered carbocyclic ring substituted with —(CR²¹R²²)ₙ—Z or —(C₂-C₆ alkenylene)-Z; n is 1, 2, 3, or 4;
Z is —ONR²³R²⁴, —OP(O)OH(CH₂)ₘCO₂R²³, —OP(S)OH(CH₂)ₘCO₂R²³, —P(O)(OH)₂, —P(O)(OH)(OCH₃), —P(O)(OH)(OCD₃), —SO₂(CH₂)ₘP(O)(OH)₂, —SO₂NR²³R²⁵, —NR²³R²⁴, or —NR²³SO₂R²⁵; R²¹ and R²² either are independently hydrogen or C₁-C₆ alkyl, or R²¹ and R²² together form an oxo group; R²³ is hydrogen or C₁-C₆ alkyl, R²⁴ is —SO₂R²⁵ or —C(O)R²⁵, or R²³ and R²⁴ together with the nitrogen to which they are attached form a substituted or unsubstituted heterocyclic ring; R²⁵ is C₁-C₆ alkyl; and m is 1, 2, 3, or 4. In some embodiments, R¹ is an aryl. In some embodiments, the aryl is a phenyl.

In some embodiments, the 5′-stabilized end cap has the structure of Formula (Ib):

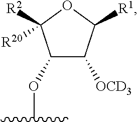

wherein
R¹ is a nucleobase, aryl, heteroaryl, or H,

### R² is

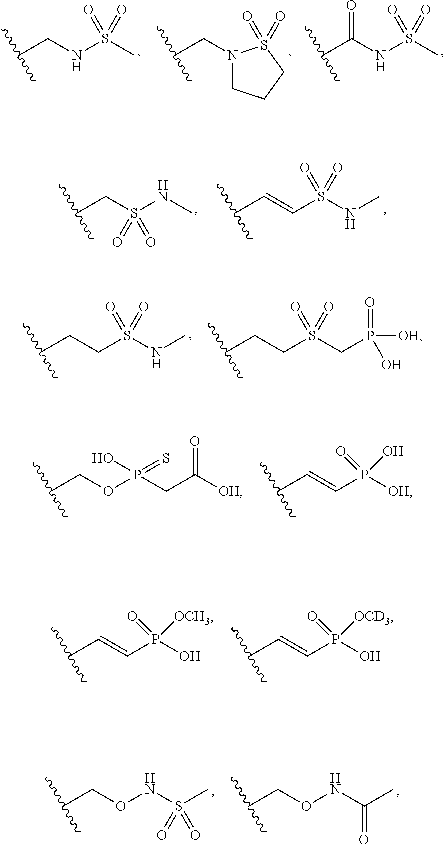

—CH═CD-Z, —CD=CH—Z, —CD=CD-Z, —(CR²¹R²²)ₙ—Z, or —(C₂-C₆ alkenylene)-Z and R²⁰ is hydrogen; or
R² and R²⁰ together form a 3- to 7-membered carbocyclic ring substituted with —(CR²¹R²²)ₙ—Z or —(C₂-C₆ alkenylene)-Z;
n is 1, 2, 3, or 4;
Z is —ONR²³R²⁴, —OP(O)OH(CH₂)ₘCO₂R²³, —OP(S)OH(CH₂)ₘCO₂R²³, —P(O)(OH)₂, —P(O)(OH)(OCH₃), —P(O)(OH)(OCD₃), —SO₂(CH₂)ₘP(O)(OH)₂, —SO₂NR²³R²⁵, —NR²³R²⁴,
R²¹ and R²² are independently hydrogen or C₁-C₆ alkyl; R²¹ and R²² together form an oxo group;
R²³ is hydrogen or C₁-C₆ alkyl;
R²⁴ is —SO₂R²⁵ or —C(O)R²⁵; or
R²³ and R²⁴ together with the nitrogen to which they are attached form a substituted or unsubstituted heterocyclic ring;
R²⁵ is C₁-C₆ alkyl; and
m is 1, 2, 3, or 4.

In some embodiments, the 5′-stabilized end cap is selected from the group consisting of Formula (1) to Formula (15), Formula (9X) to Formula (12X), and Formula (9Y) to Formula (12Y):

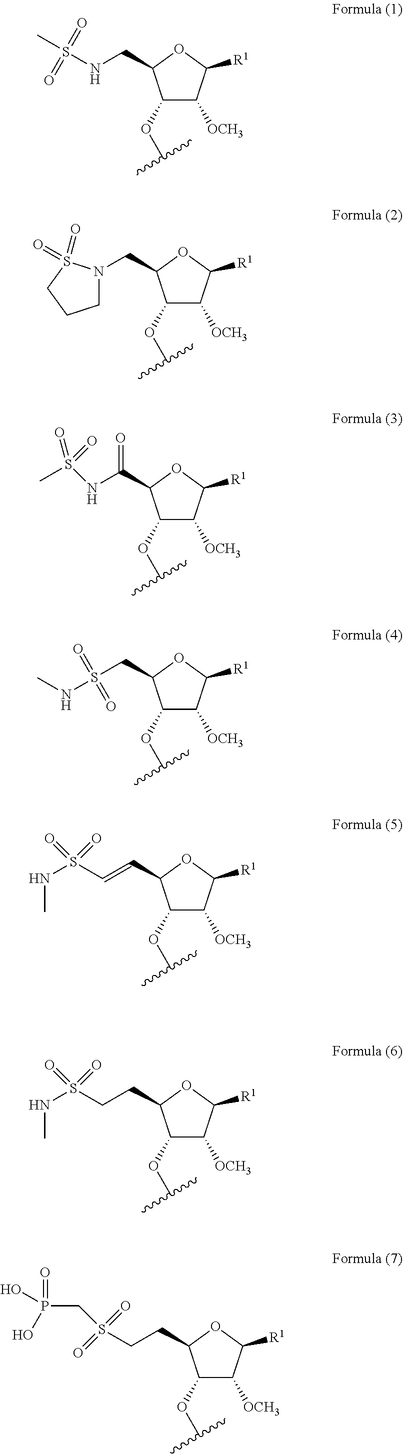

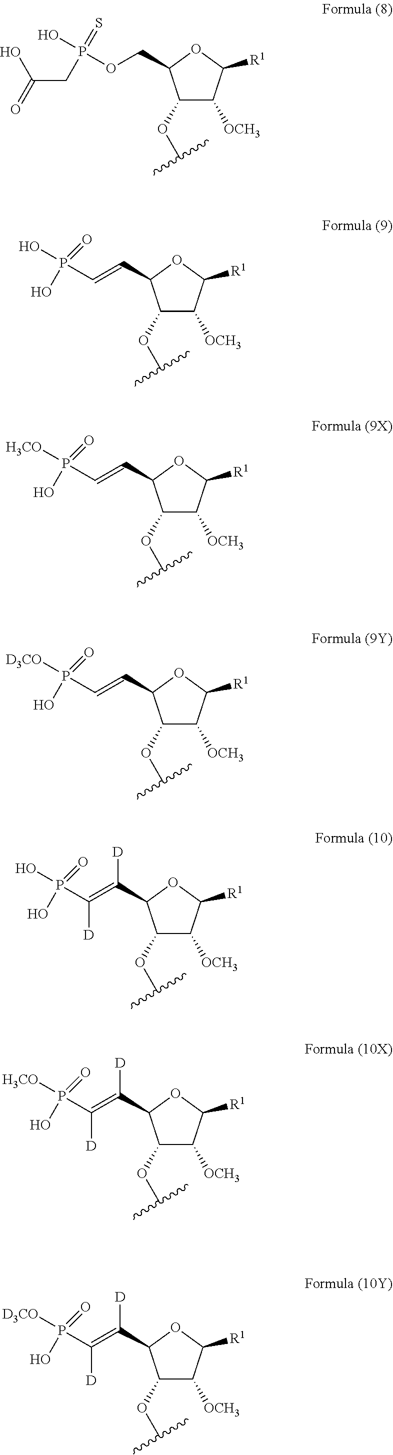

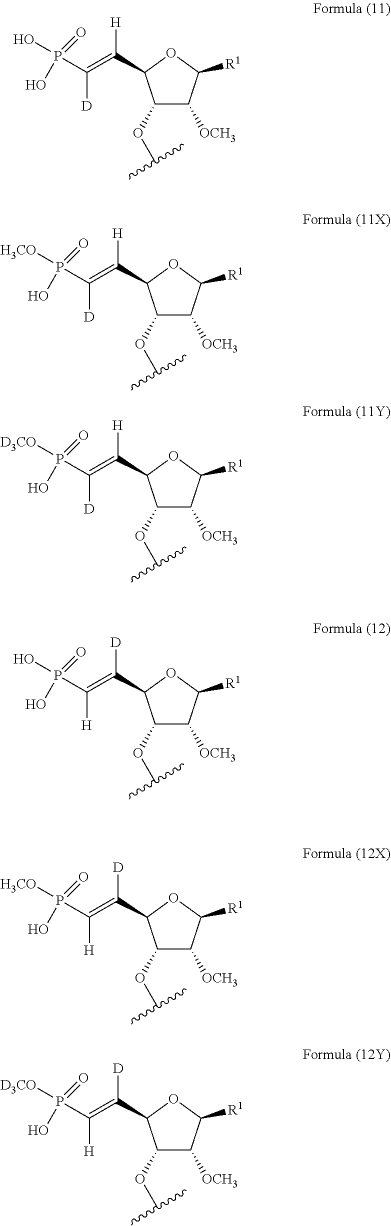

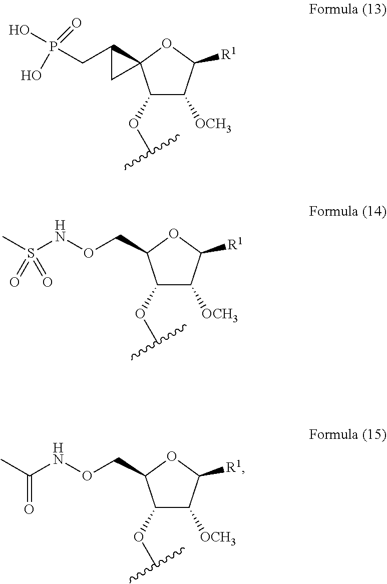

wherein R¹ is a nucleobase, aryl, heteroaryl, or H.

In some embodiments, the 5′-stabilized end cap is selected from the group consisting of Formulas (1A)-(15A), Formulas (9B)-(12B), Formulas (9AX)-(12AX), Formulas (9AY)-(12AY), Formulas (9BX)-(12BX), and Formulas (9BY)-(12BY):

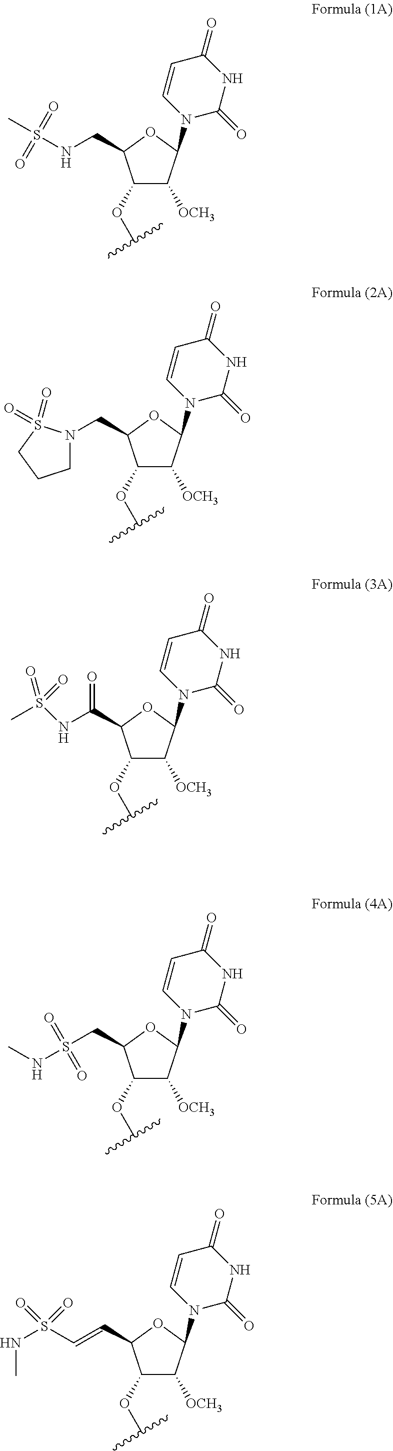 [SMILES: *O[C@H]1[C@@H](OC)[C@H](n2ccc(=O)[nH]c2=O)O[C@@H]1CNS(C)(=O)=O ; *O[C@H]1[C@@H](OC)[C@H](n2ccc(=O)[nH]c2=O)O[C@@H]1CN1CCCS1(=O)=O ; *O[C@H]1[C@@H](OC)[C@H](n2ccc(=O)[nH]c2=O)O[C@@H]1C(=O)NS(C)(=O)=O ; *O[C@H]1[C@@H](OC)[C@H](n2ccc(=O)[nH]c2=O)O[C@@H]1CS(=O)(=O)NC ; *O[C@H]1[C@@H](OC)[C@H](n2ccc(=O)[nH]c2=O)O[C@@H]1/C=C/S(=O)(=O)NC]

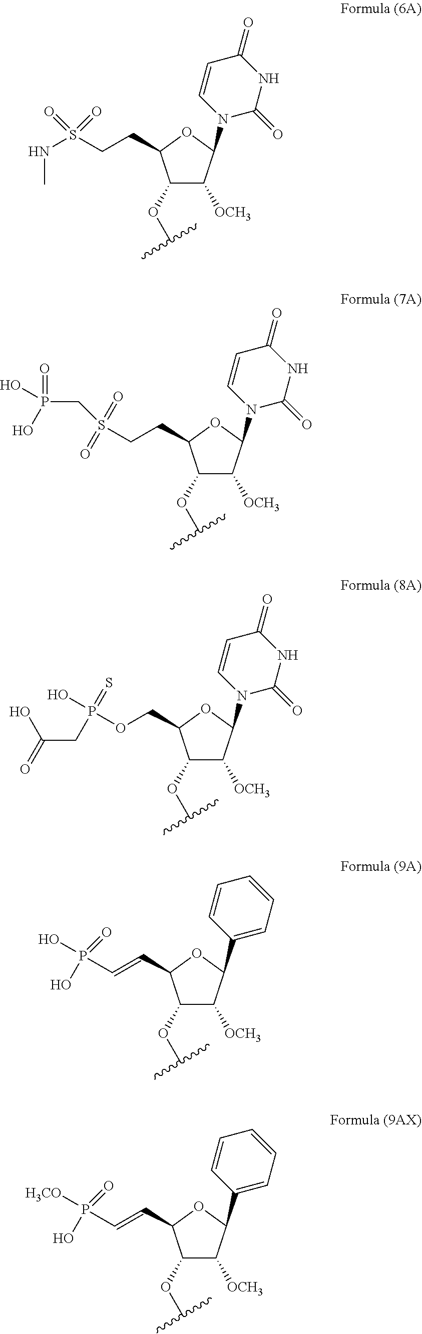 [SMILES: *O[C@H]1[C@@H](OC)[C@H](n2ccc(=O)[nH]c2=O)O[C@@H]1CCS(=O)(=O)NC ; *O[C@H]1[C@@H](OC)[C@H](n2ccc(=O)[nH]c2=O)O[C@@H]1CCS(=O)(=O)CP(=O)(O)O ; *O[C@H]1[C@@H](OC)[C@H](n2ccc(=O)[nH]c2=O)O[C@@H]1COP(O)(=S)CC(=O)O ; *O[C@H]1[C@@H](OC)[C@H](c2ccccc2)O[C@@H]1/C=C/P(=O)(O)O ; *O[C@H]1[C@@H](OC)[C@H](c2ccccc2)O[C@@H]1/C=C/P(=O)(O)OC]

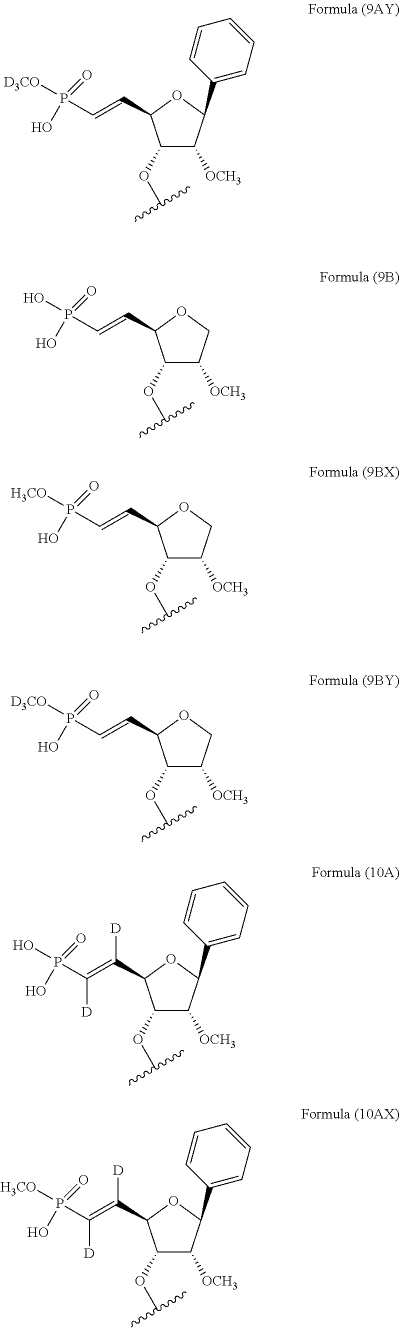

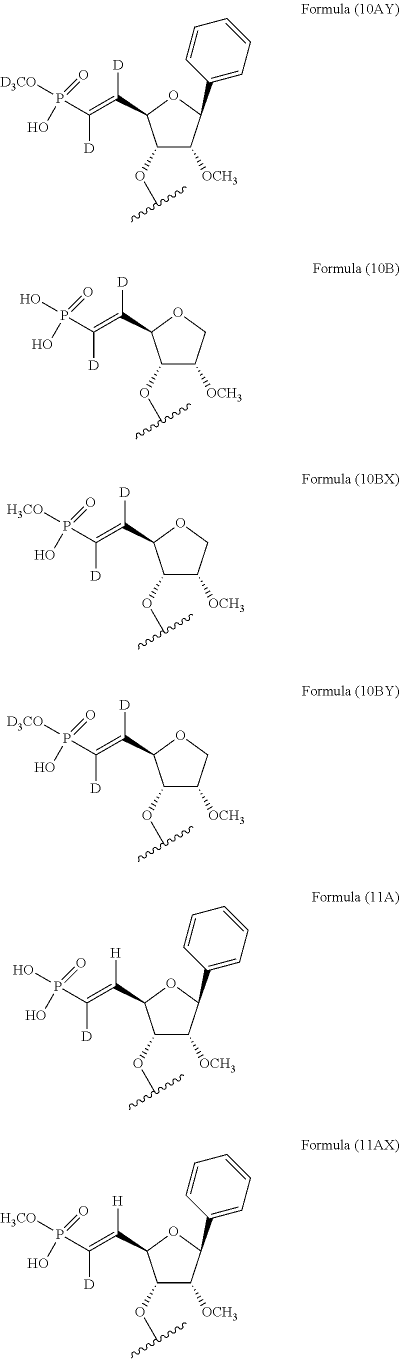

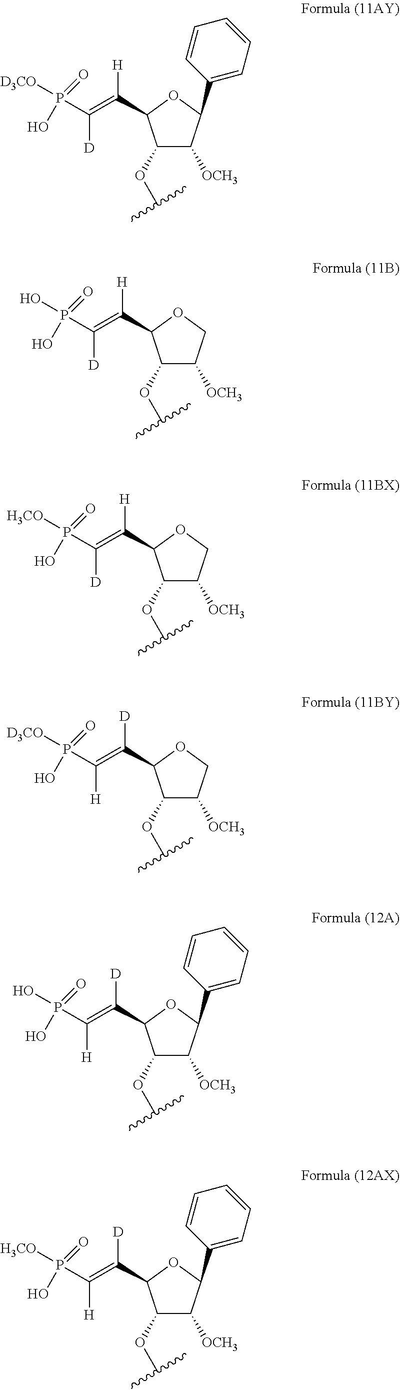

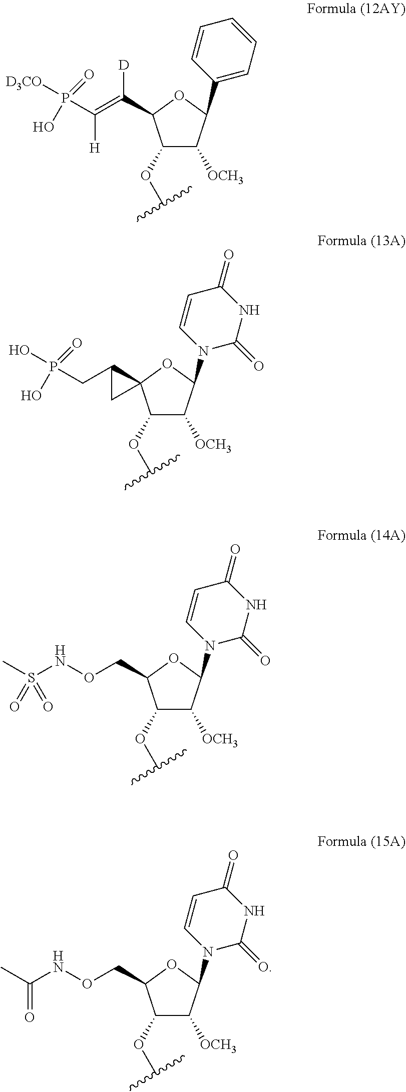

In some embodiments, the 5′-stabilized end cap can be attached to the 5′ end of the antisense strand. In some embodiments, the 5′-stabilized end cap can be attached to the 5′ end of the antisense strand via one or more linkers independently selected from a phosphodiester linker, phosphorothioate linker, phosphoramidite (HEG) linker, triethylene glycol (TEG) linker, or phosphorodithioate linker.

In some embodiments, the sense strand consists of 21 nucleotides. In some embodiments, 2′-O-methyl nucleotides are at positions 18-21 from the 5′ end of the sense strand.

In some embodiments, the antisense strand consists of 23 nucleotides. In some embodiments, 2′-O-methyl nucleotides are at positions 18-23 from the 5′ end of the antisense strand.

In another aspect, the present disclosure provides, a siNA selected from ds-siNA-005; ds-siNA-006; ds-siNA-007; ds-siNA-008; ds-siNA-009; ds-siNA-010; ds-siNA-011; ds-siNA-012; ds-siNA-013; ds-siNA-014; ds-siNA-015; ds-siNA-016; ds-siNA-017; ds-siNA-018; ds-siNA-019; ds-siNA-020; ds-siNA-021; ds-siNA-022; ds-siNA-023; ds-siNA-024; ds-siNA-025; ds-siNA-026; ds-siNA-027; ds-siNA-028; ds-siNA-029; ds-siNA-030; ds-siNA-031; ds-siNA-032; ds-siNA-033; ds-siNA-034; ds-siNA-035; ds-siNA-036; ds-siNA-037; ds-siNA-038; ds-siNA-039; ds-siNA-040; ds-siNA-041; ds-siNA-042; ds-siNA-043; ds-siNA-044; ds-siNA-045; ds-siNA-046; ds-siNA-047; ds-siNA-048; ds-siNA-049; ds-siNA-050; ds-siNA-051; ds-siNA-052; ds-siNA-053; ds-siNA-054; ds-siNA-055; ds-siNA-056; ds-siNA-057; ds-siNA-058; ds-siNA-059; ds-siNA-060; ds-siNA-061; ds-siNA-062; ds-siNA-063; ds-siNA-064; ds-siNA-065; ds-siNA-066; ds-siNA-067; ds-siNA-068; ds-siNA-069; ds-siNA-070; ds-siNA-071; ds-siNA-072; ds-siNA-073; ds-siNA-074; ds-siNA-075; ds-siNA-076; ds-siNA-077; ds-siNA-078; ds-siNA-079; ds-siNA-080; ds-siNA-081; ds-siNA-082; ds-siNA-083; ds-siNA-084; ds-siNA-085; ds-siNA-086; ds-siNA-087; ds-siNA-088; ds-siNA-089; ds-siNA-090; ds-siNA-091; ds-siNA-092; ds-siNA-093; ds-siNA-094; ds-siNA-095; ds-siNA-096; ds-siNA-097; ds-siNA-098; ds-siNA-099; ds-siNA-100; ds-siNA-101; ds-siNA-102; ds-siNA-103; ds-siNA-104; ds-siNA-105; ds-siNA-106; ds-siNA-107; ds-siNA-108; ds-siNA-109; ds-siNA-110; ds-siNA-111; ds-siNA-112; ds-siNA-113; ds-siNA-114; ds-siNA-115; ds-siNA-116; ds-siNA-117; ds-siNA-118; ds-siNA-119; ds-siNA-120; ds-siNA-121; ds-siNA-122; ds-siNA-123; ds-siNA-124; ds-siNA-125; ds-siNA-126; ds-siNA-127; ds-siNA-128; ds-siNA-129; ds-siNA-130; ds-siNA-131; ds-siNA-132; ds-siNA-133; ds-siNA-134; ds-siNA-135; ds-siNA-136; ds-siNA-137; ds-siNA-138; ds-siNA-139; ds-siNA-140; ds-siNA-141; ds-siNA-142; ds-siNA-143; ds-siNA-144; ds-siNA-145; ds-siNA-146; ds-siNA-147; ds-siNA-148; ds-siNA-149; ds-siNA-150; ds-siNA-151; ds-siNA-152; ds-siNA-153; ds-siNA-154; ds-siNA-155; ds-siNA-156; ds-siNA-157; ds-siNA-158; ds-siNA-159; ds-siNA-160; ds-siNA-161; ds-siNA-162; ds-siNA-163; ds-siNA-164; ds-siNA-165; ds-siNA-166; ds-siNA-167; ds-siNA-168; ds-siNA-169; ds-siNA-170; ds-siNA-171; ds-siNA-172; ds-siNA-173; ds-siNA-174; ds-siNA-175; ds-siNA-176; ds-siNA-177; ds-siNA-178; ds-siNA-179; ds-siNA-180; ds-siNA-181; ds-siNA-182; ds-siNA-183; ds-siNA-184; ds-siNA-185; ds-siNA-186; ds-siNA-187; ds-siNA-188; ds-siNA-189; ds-siNA-190; ds-siNA-191; ds-siNA-192; ds-siNA-193; ds-siNA-194; ds-siNA-195; ds-siNA-196; ds-siNA-197; ds-siNA-198; ds-siNA-199; ds-siNA-200; ds-siNA-201; ds-siNA-202; ds-siNA-203; ds-siNA-204; ds-siNA-205; ds-siNA-206; ds-siNA-207; ds-siNA-208; ds-siNA-209; ds-siNA-210; ds-siNA-211; ds-siNA-212; ds-siNA-213; ds-siNA-214; ds-siNA-215; ds-siNA-216; ds-siNA-217; ds-siNA-218; ds-siNA-219; ds-siNA-220; ds-siNA-221; and ds-siNA-222.

In some embodiments, the siNA is selected from ds-siNA-196 (sense and antisense respectively comprising SEQ ID NOs: 4578 and 4800), ds-siNA-197 (sense and antisense respectively comprising SEQ ID NOs: 4579 and 4801), ds-siNA-198 (sense and antisense respectively comprising SEQ ID NOs: 4580 and 4802), ds-siNA-199 (sense and antisense respectively comprising SEQ ID NOs: 4581 and 4803), ds-siNA-217 (sense and antisense respectively comprising SEQ ID NOs: 4599 and 4821), ds-siNA-218 (sense and antisense respectively comprising SEQ ID NOs: 4600 and 4822), ds-siNA-219 (sense and antisense respectively comprising SEQ ID NOs: 4601 and 4823), ds-siNA-220 (sense and antisense respectively comprising SEQ ID NOs: 4602 and 4824), ds-siNA-221 (sense and antisense respectively comprising SEQ ID NOs: 4603 and 4825), and ds-siNA-222 (sense and antisense respectively comprising SEQ ID NOs: 4604 and 4826).

In some embodiments, the siNA is selected from ds-siNA-196 (sense and antisense respectively comprising SEQ ID NOs: 4578 and 4800), ds-siNA-197 (sense and antisense respectively comprising SEQ ID NOs: 4579 and 4801), ds-siNA-198 (sense and antisense respectively comprising SEQ ID NOs: 4580 and 4802), and ds-siNA-199 (sense and antisense respectively comprising SEQ ID NOs: 4581 and 4803).

In some embodiments, the siNA is selected from, ds-siNA-217 (sense and antisense respectively comprising SEQ ID NOs: 4599 and 4821), ds-siNA-218 (sense and antisense respectively comprising SEQ ID NOs: 4600 and 4822), ds-siNA-219 (sense and antisense respectively comprising SEQ ID NOs: 4601 and 4823), ds-siNA-220 (sense and antisense respectively comprising SEQ ID NOs: 4602 and 4824), ds-siNA-221 (sense and antisense respectively comprising SEQ ID NOs: 4603 and 4825), and ds-siNA-222 (sense and antisense respectively comprising SEQ ID NOs: 4604 and 4826).

In another aspect, the present disclosure provides pharmaceutical compositions comprising at least one siNA according to any one of the embodiments described herein and a pharmaceutically acceptable carrier or diluent.

In some embodiments, the pharmaceutical composition can comprise two or more siNA according to any of the embodiments described herein.

In another aspect, the present disclosure provides methods for treating a disease in a subject in need thereof, comprising administering the subject a pharmaceutical composition according to any of the embodiments described herein.

In another aspect, the present disclosure provides uses of a ds-siRNA according to any of the embodiments described herein in the manufacture of a medicament for treating a disease.

In another aspect, the present disclosure provides methods for treating a disease in a subject in need thereof, comprising administering the subject a siNA according to any of the embodiments described herein. In some embodiments, wherein the disease is a viral disease. In some embodiments, the viral disease is caused by an RNA virus. In some embodiments, the RNA virus is a single-stranded RNA virus (ssRNA virus). In some embodiments, the ssRNA virus is a positive-sense single-stranded RNA virus ((+)ssRNA virus). In some embodiments, the (+)ssRNA virus is a coronavirus. In some embodiments, the coronavirus is a β-coronavirus. In some embodiments, the β-coronavirus is severe acute respiratory syndrome coronavirus 2 (SARS-CoV-2) (also known by the provisional name 2019 novel coronavirus, or 2019-nCoV), human coronavirus OC43 (hCoV-OC43), Middle East respiratory syndrome-related coronavirus (MERS-CoV, also known by the provisional name 2012 novel coronavirus, or 2012-nCoV), or severe acute respiratory syndrome-related coronavirus (SARS-CoV, also known as SARS-CoV-1). In some embodiments, the β-coronavirus is SARS-CoV-2. In some embodiments, the (3-coronavirus is SARS-CoV. In some embodiments, the β-coronavirus is MERS-CoV. In some embodiments, the β-coronavirus is hCoV-OC43.

In some embodiments, the disease is a respiratory disease. In some embodiments, the respiratory disease is viral pneumonia. In some embodiments, the respiratory disease is an acute respiratory infection. In some embodiments, the respiratory disease is a cold. In some embodiments, the respiratory disease is severe acute respiratory syndrome (SARS). In some embodiments, the respiratory disease is Middle East respiratory syndrome (MERS). In some embodiments, the disease is coronavirus disease 2019 (COVID-19). In some embodiments, the respiratory disease causes one or more symptoms selected from coughing, sore throat, runny nose, sneezing, headache, fever, shortness of breath, myalgia, abdominal pain, fatigue, difficulty breathing, persistent chest pain or pressure, difficulty waking, loss of smell and taste, muscle or joint pain, chills, nausea or vomiting, nasal congestion, diarrhea, haemoptysis, conjunctival congestion, sputum production, chest tightness, and palpitations. In some embodiments, the respiratory disease can cause complications selected from sinusitis, otitis media, pneumonia, acute respiratory distress syndrome, disseminated intravascular coagulation, pericarditis, and kidney failure. In some embodiments, the respiratory disease is idiopathic.

In another aspect, the present disclosure provides methods of treating a β-coronavirus-caused disease in a subject in need thereof, comprising administering the subject a siNA comprising a sense strand that is 15 to 30 nucleotides in length, wherein the sense strand is at least about 60%, 65%, 70%, 75%, 80%, 85%, 90%, 95%, or 100% identical to a sequence within a region of either two, three, or four of SEQ ID NOs: 2407, 2408, 2409, and 2410. In some embodiments, the sense strand is identical to an RNA sequence corresponding to a region of each of SEQ ID NOs: 2407, 2408, 2409, and 2410. In some embodiments, the sense strand is selected from the group consisting of sequences corresponding to SEQ ID NOs: 1-1203 and 2411-3392.

In another aspect, the present disclosure provides methods of treating a β-coronavirus-caused disease in a subject in need thereof, comprising administering the subject a siNA comprising antisense strand that is 15 to 30 nucleotides in length, wherein the antisense strand is complementary to a sequence within a region of either two, three, or four of SEQ ID NOs: 2407, 2408, 2409, and 2410. In some embodiments, the second nucleotide sequence is at least about 60%, 65%, 70%, 75%, 80%, 85%, 90%, 95%, or 100% complementary to an RNA sequence corresponding to a region of each of SEQ ID NOs: 2407, 2408, 2409, and 2410. In some embodiments, the antisense strand comprises a sequence corresponding to one of SEQ ID NOs: 1204-2406 and 3393-4374.

In another aspect, the present disclosure provides methods of treating a β-coronavirus-caused disease in a subject in need thereof, comprising administering the subject a siNA comprising a sense strand that is 15 to 30 nucleotides in length, wherein the sense strand is at least about 60%, 65%, 70%, 75%, 80%, 85%, 90%, 95%, or 100% identical to a sequence within a region of either two, three, or four of the genomes of severe acute respiratory syndrome coronavirus 2 (SARS-CoV-2), human coronavirus OC43 (hCoV-OC43), Middle East respiratory syndrome-related coronavirus (MERS-CoV), and severe acute respiratory syndrome-related coronavirus (SARS-CoV). In some embodiments, the sense strand is identical to a sequence within a region of each of the genomes of severe acute respiratory syndrome coronavirus 2 (SARS-CoV-2), human coronavirus OC43 (hCoV-OC43), Middle East respiratory syndrome-related coronavirus (MERS-CoV), and severe acute respiratory syndrome-related coronavirus (SARS-CoV).

In another aspect, the present disclosure provides methods of treating a β-coronavirus-caused disease in a subject in need thereof, comprising administering the subject a siNA comprising an antisense strand that is 15 to 30 nucleotides in length, wherein the antisense strand is at least about 60%, 65%, 70%, 75%, 80%, 85%, 90%, 95%, or 100% complementary to a sequence within a region of either two, three, or four of the genomes of severe acute respiratory syndrome coronavirus 2 (SARS-CoV-2), human coronavirus OC43 (hCoV-OC43), Middle East respiratory syndrome-related coronavirus (MERS-CoV), and severe acute respiratory syndrome-related coronavirus (SARS-CoV). In some embodiments, the second nucleotide sequence is complementary to a sequence within a region of each of the genomes of severe acute respiratory syndrome coronavirus 2 (SARS-CoV-2), human coronavirus OC43 (hCoV-OC43), Middle East respiratory syndrome-related coronavirus (MERS-CoV), and severe acute respiratory syndrome-related coronavirus (SARS-CoV).

A method of treating a β-coronavirus-caused disease in a subject in need thereof, comprising administering the subject a siNA selected from ds-siNA-005; ds-siNA-006; ds-siNA-007; ds-siNA-008; ds-siNA-009; ds-siNA-010; ds-siNA-011; ds-siNA-012; ds-siNA-013; ds-siNA-014; ds-siNA-015; ds-siNA-016; ds-siNA-017; ds-siNA-018; ds-siNA-019; ds-siNA-020; ds-siNA-021; ds-siNA-022; ds-siNA-023; ds-siNA-024; ds-siNA-025; ds-siNA-026; ds-siNA-027; ds-siNA-028; ds-siNA-029; ds-siNA-030; ds-siNA-031; ds-siNA-032; ds-siNA-033; ds-siNA-034; ds-siNA-035; ds-siNA-036; ds-siNA-037; ds-siNA-038; ds-siNA-039; ds-siNA-040; ds-siNA-041; ds-siNA-042; ds-siNA-043; ds-siNA-044; ds-siNA-045; ds-siNA-046; ds-siNA-047; ds-siNA-048; ds-siNA-049; ds-siNA-050; ds-siNA-051; ds-siNA-052; ds-siNA-053; ds-siNA-054; ds-siNA-055; ds-siNA-056; ds-siNA-057; ds-siNA-058; ds-siNA-059; ds-siNA-060; ds-siNA-061; ds-siNA-062; ds-siNA-063; ds-siNA-064; ds-siNA-065; ds-siNA-066; ds-siNA-067; ds-siNA-068; ds-siNA-069; ds-siNA-070; ds-siNA-071; ds-siNA-072; ds-siNA-073; ds-siNA-074; ds-siNA-075; ds-siNA-076; ds-siNA-077; ds-siNA-078; ds-siNA-079; ds-siNA-080; ds-siNA-081; ds-siNA-082; ds-siNA-083; ds-siNA-084; ds-siNA-085; ds-siNA-086; ds-siNA-087; ds-siNA-088; ds-siNA-089; ds-siNA-090; ds-siNA-091; ds-siNA-092; ds-siNA-093; ds-siNA-094; ds-siNA-095; ds-siNA-096; ds-siNA-097; ds-siNA-098; ds-siNA-099; ds-siNA-100; ds-siNA-101; ds-siNA-102; ds-siNA-103; ds-siNA-104; ds-siNA-105; ds-siNA-106; ds-siNA-107; ds-siNA-108; ds-siNA-109; ds-siNA-110; ds-siNA-111; ds-siNA-112; ds-siNA-113; ds-siNA-114; ds-siNA-115; ds-siNA-116; ds-siNA-117; ds-siNA-118; ds-siNA-119; ds-siNA-120; ds-siNA-121; ds-siNA-122; ds-siNA-123; ds-siNA-124; ds-siNA-125; ds-siNA-126; ds-siNA-127; ds-siNA-128; ds-siNA-129; ds-siNA-130; ds-siNA-131; ds-siNA-132; ds-siNA-133; ds-siNA-134; ds-siNA-135; ds-siNA-136; ds-siNA-137; ds-siNA-138; ds-siNA-139; ds-siNA-140; ds-siNA-141; ds-siNA-142; ds-siNA-143; ds-siNA-144; ds-siNA-145; ds-siNA-146; ds-siNA-147; ds-siNA-148; ds-siNA-149; ds-siNA-150; ds-siNA-151; ds-siNA-152; ds-siNA-153; ds-siNA-154; ds-siNA-155; ds-siNA-156; ds-siNA-157; ds-siNA-158; ds-siNA-159; ds-siNA-160; ds-siNA-161; ds-siNA-162; ds-siNA-163; ds-siNA-164; ds-siNA-165; ds-siNA-166; ds-siNA-167; ds-siNA-168; ds-siNA-169; ds-siNA-170; ds-siNA-171; ds-siNA-172; ds-siNA-173; ds-siNA-174; ds-siNA-175; ds-siNA-176; ds-siNA-177; ds-siNA-178; ds-siNA-179; ds-siNA-180; ds-siNA-181; ds-siNA-182; ds-siNA-183; ds-siNA-184; ds-siNA-185; ds-siNA-186; ds-siNA-187; ds-siNA-188; ds-siNA-189; ds-siNA-190; ds-siNA-191; ds-siNA-192; ds-siNA-193; ds-siNA-194; ds-siNA-195; ds-siNA-196; ds-siNA-197; ds-siNA-198; ds-siNA-199; ds-siNA-200; ds-siNA-201; ds-siNA-202; ds-siNA-203; ds-siNA-204; ds-siNA-205; ds-siNA-206; ds-siNA-207; ds-siNA-208; ds-siNA-209; ds-siNA-210; ds-siNA-211; ds-siNA-212; ds-siNA-213; ds-siNA-214; ds-siNA-215; ds-siNA-216; ds-siNA-217; ds-siNA-218; ds-siNA-219; ds-siNA-220; ds-siNA-221; and ds-siNA-222. In some embodiments, the siNA is selected from ds-siNA-196 (sense and antisense respectively comprising SEQ ID NOs: 4578 and 4800), ds-siNA-197 (sense and antisense respectively comprising SEQ ID NOs: 4579 and 4801), ds-siNA-198 (sense and antisense respectively comprising SEQ ID NOs: 4580 and 4802), ds-siNA-199 (sense and antisense respectively comprising SEQ ID NOs: 4581 and 4803), ds-siNA-217 (sense and antisense respectively comprising SEQ ID NOs: 4599 and 4821), ds-siNA-218 (sense and antisense respectively comprising SEQ ID NOs: 4600 and 4822), ds-siNA-219 (sense and antisense respectively comprising SEQ ID NOs: 4601 and 4823), ds-siNA-220 (sense and antisense respectively comprising SEQ ID NOs: 4602 and 4824), ds-siNA-221 (sense and antisense respectively comprising SEQ ID NOs: 4603 and 4825), and ds-siNA-222 (sense and antisense respectively comprising SEQ ID NOs: 4604 and 4826). In some embodiments, the siNA is selected from ds-siNA-196 (sense and antisense respectively comprising SEQ ID NOs: 4578 and 4800), ds-siNA-197 (sense and antisense respectively comprising SEQ ID NOs: 4579 and 4801), ds-siNA-198 (sense and antisense respectively comprising SEQ ID NOs: 4580 and 4802), and ds-siNA-199 (sense and antisense respectively comprising SEQ ID NOs: 4581 and 4803). In some embodiments, the siNA is selected from, ds-siNA-217 (sense and antisense respectively comprising SEQ ID NOs: 4599 and 4821), ds-siNA-218 (sense and antisense respectively comprising SEQ ID NOs: 4600 and 4822), ds-siNA-219 (sense and antisense respectively comprising SEQ ID NOs: 4601 and 4823), ds-siNA-220 (sense and antisense respectively comprising SEQ ID NOs: 4602 and 4824), ds-siNA-221 (sense and antisense respectively comprising SEQ ID NOs: 4603 and 4825), and ds-siNA-222 (sense and antisense respectively comprising SEQ ID NOs: 4604 and 4826). In some embodiments, the (3-coronavirus can be SARS-CoV-2. In some embodiments, the β-coronavirus-caused disease can be COVID-19.

In some embodiments of the disclosed methods and uses, the subject is a mammal. In some embodiments, the subject is a human. In some embodiments, the subject is a non-human primate. In some embodiments, the subject is a cat. In some embodiments, the subject is a camel.

In some embodiments of the disclosed methods and uses, the siNA is administered intravenously, subcutaneously, or via inhalation.

In some embodiments of the disclosed methods and uses, the subject has been treated with one or more additional coronavirus treatment agents. In some embodiments of the disclosed methods, the subject is concurrently treated with one or more additional coronavirus treatment agents.

The foregoing general description and following detailed description are exemplary and explanatory and are intended to provide further explanation of the disclosure as claimed. Other objects, advantages, and novel features will be readily apparent to those skilled in the art from the following brief description of the drawings and detailed description of the disclosure.

### BRIEF DESCRIPTION OF THE DRAWINGS

FIG. 1 (FIG. 1) shows the coronaviridae family and its four genera (top panel) and the full length genome of NCBI 407 (bottom panel), which encodes 28 proteins across multiple open reading frames (ORF s).

FIG. 2 (FIG. 2) shows the percent identity between multiple coronavirus, including sudden acute respiratory syndrome coronavirus (SARS-CoV), Middle East respiratory syndrome coronavirus (MERS-CoV), and human coronavirus OC43 (top panel), and an alignment of the highly similar region of the genomes encodings non-structural protein 8 (nsp8) to non-structural protein (nsp15) (bottom panel).

FIG. 3 (FIG. 3) shows details of nsp8-nsp15.

FIG. 4 (FIG. 4) shows an exemplary siNA molecule.

FIG. 5 (FIG. 5) shows an exemplary siNA molecule.

FIGS. 6A-6I (FIGS. 6A-6I) show exemplary double-stranded siNA molecules.

### DETAILED DESCRIPTION

Disclosed herein are short interfering nucleic acid (siNA) molecules. In some embodiments, the siNA is a double-stranded siNA (ds-siNA). In some embodiments, the ds-siNA comprises a sense strand and an antisense strand. In some embodiments, the ds-siNA comprises (a) a sense strand comprising a first nucleotide sequence, wherein the first nucleotide sequence is 15 to 30 nucleotides in length; and (b) an antisense strand comprising a second nucleotide sequence, wherein the second nucleotide sequence is 15 to 30 nucleotides in length and comprises a nucleotide sequence that is the reverse complement of the first nucleotide sequence.

Further disclosed herein are pharmaceutical compositions comprising the ds-siNA according to any one of the embodiments described herein and a pharmaceutically acceptable carrier or diluent. In some embodiments the disclosed compositions may comprise two or more ds-siNA according to any of the embodiments described herein.

Further disclosed herein is a method for treating a disease in a subject in need thereof, comprising administering the subject one or more siNA or pharmaceutical compositions of any of the embodiments described herein. In some embodiments, the disease is a viral infection, such as a coronavirus infection (e.g., COVID-19).

Further disclosed herein is the use of one or more ds-siRNA according to any of the embodiments described herein in the manufacture of a medicament for treating a disease, such as a viral infection or, more specifically, a coronavirus infection (e.g., COVID-19).

Further disclosed herein is a method for treating a disease in a subject in need thereof, comprising administering the subject one or more ds-siNA or pharmaceutical compositions of any of the embodiments described herein.

Further disclosed herein is a method of treating a β-coronavirus-caused disease (e.g., COVID-19) in a subject in need thereof, comprising administering the subject one or more ds-siNA according to any of the embodiments described herein.

As described in more detail below, the disclose siNA molecules may comprise modified nucleotides. The modified nucleotides may be selected from 2′-O-methyl nucleotides and 2′-fluoro nucleotides. The siNA molecules described herein may comprise 1, 2, 3, 4, 5, 6, 7, 8, 9, or 10 or more phosphorothioate internucleoside linkages. The siNA molecules described herein may comprise at least one phosphorylation blocker. The siNA molecules described herein may comprise a 5′-stabilized end cap. The siNA molecules described herein may comprise one or more blunt ends. The siNA molecules described herein may comprise one or more overhangs.

Further, the disclosed siNA molecules may comprise (a) a phosphorylation blocker; and (b) a siNA. The siNA may comprise at least 5 nucleotides. The nucleotides may be modified nucleotides, non-modified nucleotides, or any combination thereof. The nucleotides may be ribonucleotides, deoxyribonucleotides, or any combination thereof. The siNA may be single-stranded. Alternatively, the siNA is double-stranded. The double-stranded siNA may comprise one or more blunt ends. The double-stranded siNA may comprise one or more overhangs. The double-stranded siNA may comprise a blunt end and an overhang.

Further, the disclosed siNA molecules may comprise (a) a conjugated moiety; and (b) a siNA. The siNA may comprise at least 5 nucleotides. The nucleotides may be modified nucleotides, non-modified nucleotides, or any combination thereof. The nucleotides may be ribonucleotides, deoxyribonucleotides, or any combination thereof. The siNA may be single-stranded. Alternatively, the siNA is double-stranded. The double-stranded siNA may comprise one or more blunt ends. The double-stranded siNA may comprise one or more overhangs. The double-stranded siNA may comprise a blunt end and an overhang.

Further, the disclosed siNA molecules may comprise (a) a 5′-stabilized end cap; and (b) a siNA. The siNA may comprise at least 5 nucleotides. The nucleotides may be modified nucleotides, non-modified nucleotides, or any combination thereof. The nucleotides may be ribonucleotides, deoxyribonucleotides, or any combination thereof. The siNA may be single-stranded. Alternatively, the siNA is double-stranded. The double-stranded siNA may comprise one or more blunt ends. The double-stranded siNA may comprise one or more overhangs. The double-stranded siNA may comprise a blunt end and an overhang.

Further, the disclosed siNA molecules may comprise (a) at least one phosphorylation blocker, conjugated moiety, or 5′-stabilized end cap; and (b) a siNA. The siNA may comprise at least 5 nucleotides. The nucleotides may be modified nucleotides, non-modified nucleotides, or any combination thereof. The nucleotides may be ribonucleotides, deoxyribonucleotides, or any combination thereof. The siNA may be single-stranded. Alternatively, the siNA is double-stranded. The double-stranded siNA may comprise one or more blunt ends. The double-stranded siNA may comprise one or more overhangs. The double-stranded siNA may comprise a blunt end and an overhang.

Exemplary siNA, which may be used to treat and/or prevent coronavirus infections (e.g., COVID-19) are also described herein.

### Definitions

It is to be understood that methods are not limited to the particular embodiments described, and as such may vary. It is also to be understood that the terminology used herein is for the purpose of describing particular embodiments only and is not intended to be limiting. The scope of the present technology will be limited only by the appended claims.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

As used in the specification and claims, the singular form “a,” “an” and “the” include singular and plural references unless the context clearly dictates otherwise.

As used herein, the term “comprising” is intended to mean that the compositions and methods include the recited elements, but not excluding others. “Consisting essentially of” when used to define compositions and methods, shall mean excluding other elements of any essential significance to the composition or method. “Consisting of” shall mean excluding more than trace elements of other ingredients for claimed compositions and substantial method steps. Embodiments defined by each of these transition terms are within the scope of this disclosure. Accordingly, it is intended that the methods and compositions can include additional steps and components (comprising) or alternatively including steps and compositions of no significance (consisting essentially of) or alternatively, intending only the stated method steps or compositions (consisting of).

As used herein, “about” means plus or minus 10% as well as the specified number. For example, “about 10” should be understood as both “10” and “9-11.”

As used herein, “optional” or “optionally” means that the subsequently described event or circumstance may or may not occur, and that the description includes instances where said event or circumstance occurs and instances where it does not.

The terms “individual,” “subject,” and “patient” are used interchangeably herein, and refer to any individual mammal, e.g., bovine, canine, feline, equine, simian, porcine, camelid, bat, or human, being treated according to the disclosed methods or uses. In preferred embodiments, the subject is a human.

As used herein, the phrases “effective amount,” “therapeutically effective amount,” and “therapeutic level” mean the siNA dosage or concentration in a subject that provides the specific pharmacological effect for which the siNA is administered in a subject in need of such treatment, i.e. to treat or prevent a coronavirus infection (e.g., MERS, SARS, or COVID-19). It is emphasized that a therapeutically effective amount or therapeutic level of an siNA will not always be effective in treating the infections described herein, even though such dosage is deemed to be a therapeutically effective amount by those of skill in the art. For convenience only, exemplary dosages, drug delivery amounts, therapeutically effective amounts, and therapeutic levels are provided below. Those skilled in the art can adjust such amounts in accordance with standard practices as needed to treat a specific subject and/or condition. The therapeutically effective amount may vary based on the route of administration and dosage form, the age and weight of the subject, and/or the subject's condition, including the type and severity of the coronavirus infection.

The terms “treatment” or “treating” as used herein with reference to a coronavirus infections refer to reducing or eliminating viral load and/or improving or ameliorating one or more symptoms of an infection such as cough, shortness of breath, body aches, chills, and/or fever.

The terms “prevent” or “preventing” as used herein with reference to a coronavirus infections refer to precluding an infection from developing in a subject exposed to a coronavirus and/or avoiding the development of one or more symptoms of an infection such as cough, shortness of breath, body aches, chills, and/or fever. “Prevention” may occur when the viral load is never allowed to exceed beyond a threshold level at which point the subject begins to feel sick or exhibit symptoms. “Prevention” may also, in some embodiments, refer to the prevention of a subsequent infection once an initial infection has been treated or cured.

As used herein, the term “pharmaceutical composition” refers to the combination of an active agent with a carrier, inert or active, making the composition especially suitable for diagnostic or therapeutic use in vivo or ex vivo.

As used herein, the term “pharmaceutically acceptable carrier” refers to any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants, see, for example, Martin, Remington's Pharmaceutical Sciences, 15th Ed., Mack Publ. Co., Easton, Pa. [1975].

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

As used herein, the term “nucleobase” refers to a nitrogen-containing biological compound that forms a nucleoside. Examples of nucleobases include, but are not limited to, thymine, uracil, adenine, cytosine, guanine, aryl, heteroaryl, and an analogue or derivative thereof.

The target gene may be any gene in a cell or virus. Here, “target gene” and “target sequence” are used synonymously.

For the purposes of the present disclosure, a DNA sequence that replaces all the U residues of an RNA sequence with T residues is “identical” to the RNA sequence, and vice versa. Accordingly, a sequence that is “identical to an RNA corresponding to” a DNA sequence constitutes the DNA sequence with all T replaced by U. The presence of modified nucleotides or 2′-deoxynucleotides in a sequence does not make a sequence not “identical to an RNA” but rather a modified RNA.

As used herein, “modified nucleotide” includes any nucleic acid or nucleic acid analogue residue that contains a modification or substitution in the chemical structure of an unmodified nucleotide base, sugar (including, but not limited to, 2′-substitution), or phosphate (including, but not limited to, alternate internucleotide linkers, such as phosphorothioates or the substitution of bridging oxygens in phosphate linkers with bridging sulfurs), or a combination thereof. Non-limiting examples of modified nucleotides are shown herein.

As used herein, the term “d2vd3 nucleotide” refers to a nucleotide comprising a 5′-stabilized end cap of Formula (10):

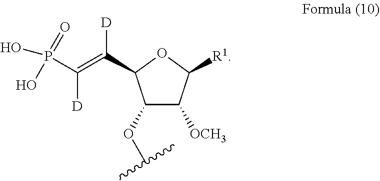

Throughout the description, where compositions are described as having, including, or comprising specific components, or where processes and methods are described as having, including, or comprising specific steps, it is contemplated that, additionally, there are compositions of the present disclosure that consist essentially of, or consist of, the recited components, and that there are processes and methods according to the present disclosure that consist essentially of, or consist of, the recited processing steps.

As a general matter, compositions specifying a percentage are by weight unless otherwise specified. Further, if a variable is not accompanied by a definition, then the previous definition of the variable controls.

### Coronaviruses and Coronavirus Infections

The siNA molecules and compositions described herein may be administered to a subject to treat a disease. Further disclosed herein are uses of any of the siNA molecules or compositions disclosed herein in the manufacture of a medicament for treating a disease.

In some embodiments of the disclosed method and uses, the disease being treated is a viral disease. In some embodiments, the viral disease is caused by an RNA virus. In some embodiments, the RNA virus is a single-stranded RNA virus (ssRNA virus). In some embodiments, the ssRNA virus is a positive-sense single-stranded RNA virus ((+)ssRNA virus). In some embodiments, the (+)ssRNA virus is a coronavirus.

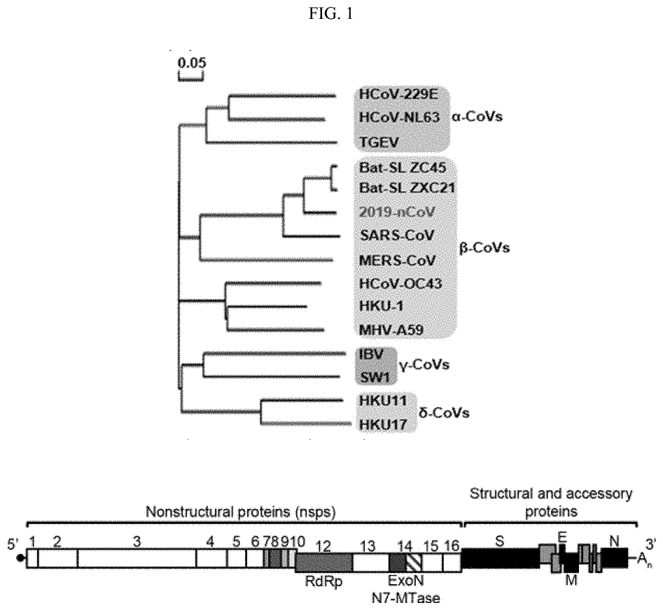

Coronaviruses are a family of viruses (i.e., the coronaviridae family) that cause respiratory infections in mammals and that comprise a genome that is roughly 30 kilobases in length. The coronaviridae family is divided into four genera and the genome encodes 28 proteins across multiple open reading frames, including 16 non-structural proteins (nsp) that are post-translationally cleaved from a polyprotein (see FIG. 1).

The coronaviridae family includes both α-coronaviruses or β-coronaviruses, which both mainly infect bats, but can also infect other mammals like humans, camels, and rabbits. β-coronaviruses have, to date, been of greater clinical importance, having caused epidemics including severe acute respiratory syndrome (SARS), Middle East respiratory syndrome (MERS), and COVID-19. Other disease-causing β-coronaviruses include OC44, and HKU1. Non-limiting examples of disease-causing α-coronaviruses include, but are not limited to, 229E and NL63.

In some embodiments, the coronavirus is a β-coronaviruses. In some embodiments, the β-coronaviruses is selected from the group consisting of severe acute respiratory syndrome coronavirus 2 (SARS-CoV-2) (also known by the provisional name 2019 novel coronavirus, or 2019-nCoV), human coronavirus OC43 (hCoV-OC43), Middle East respiratory syndrome-related coronavirus (MERS-CoV, also known by the provisional name 2012 novel coronavirus, or 2012-nCoV), and severe acute respiratory syndrome-related coronavirus (SARS-CoV, also known as SARS-CoV-1). In some embodiments, the β-coronaviruses is SARS-CoV-2, the causative agent of COVID-19.

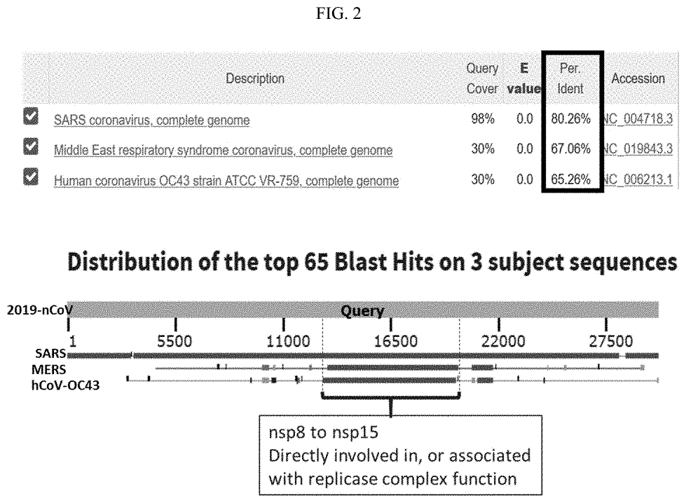

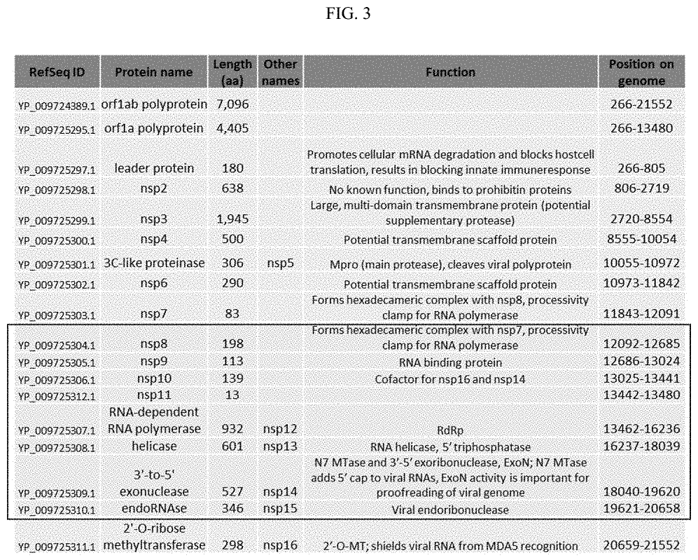

As shown in FIGS. 2 and 3, several disease-causing coronaviruses share a high degree of homology in the regions of the genome encoding non-structural proteins (nsp), and more specifically, in the region encoding nsp8-nsp15. Indeed, there is roughly 65% identity across the roughly 7 kB sequence of β-coronaviruses from about nucleotide 12900 to about nucleotide 19900 of 2019-nCoV, and some subsections of the genomic span of nsp8 to nsp15 may comprise 95% or more identity. All of the genes in this region encode non-structural proteins associated with replication. Accordingly, this segment of the genome is suitable for targeting with an siNA that can provide a broad spectrum treatment for multiple different types of coronavirus, such as MERS-CoV, SARS-CoV-1, and SARS-CoV-2.

Without wishing to be bound by theory, upon entry into a cell, any of the ds-siNA molecules disclosed herein may interact with proteins in the cell to form a RNA-Induced Silencing Complex (RISC). Once the ds-siNA is part of the RISC, the ds-siNA may be unwound to form a single-stranded siNA (ss-siNA). The ss-siNA may comprise the antisense strand of the ds-siNA. The antisense strand may bind to a complementary messenger RNA (mRNA), which results in silencing of the gene that encodes the mRNA.

In some embodiments, the target gene is a viral gene. In some embodiments, the viral gene is from an RNA virus. In some embodiments, the RNA virus is a single-stranded RNA virus (ssRNA virus). In some embodiments, the ssRNA virus is a positive-sense single-stranded RNA virus ((+)ssRNA virus). In some embodiments, the (+)ssRNA virus is a coronavirus. In some embodiments, the coronavirus is a β-coronavirus. In some embodiments, the β-coronavirus is severe acute respiratory syndrome coronavirus 2 (SARS-CoV-2) (also known by the provisional name 2019 novel coronavirus, or 2019-nCoV), human coronavirus OC43 (hCoV-OC43), Middle East respiratory syndrome-related coronavirus (MERS-CoV, also known by the provisional name 2012 novel coronavirus, or 2012-nCoV), severe acute respiratory syndrome-related coronavirus (SARS-CoV, also known as SARS-CoV-1). In some embodiments, the β-coronavirus is SARS-CoV-2.

In some embodiments, the target gene is selected from genome of SARS-CoV-2. In some embodiments, SARS-CoV-2 has a genome sequence shown in the nucleotide sequence of SEQ ID NO: 2407, which corresponds to the nucleotide sequence of GenBank Accession No. NC_045512.2, which is incorporated by reference in its entirety.

In some embodiments, the target gene is selected from genome of SARS-CoV. In some embodiments, SARS-CoV has a genome sequence shown in the nucleotide sequence of SEQ ID NO: 2408, which corresponds to the nucleotide sequence of GenBank Accession No. NC_004718.3, which is incorporated by reference in its entirety.

In some embodiments, the target gene is selected from the genome of MERS-CoV. In some embodiments, MERS-CoV has a genome sequence shown in the nucleotide sequence of SEQ ID NO: 2409, which corresponds to the nucleotide sequence of GenBank Accession No. NC_019843.3, which is incorporated by reference in its entirety.

In some embodiments, the target gene is selected from the genome of hCoV-OC43. In some embodiments, hCoV-OC43 has a genome sequence shown in the nucleotide sequence of SEQ ID NO: 2410, which corresponds to the nucleotide sequence of GenBank Accession No. NC_006213.1, which is incorporated by reference in its entirety.

Short Interfering Nucleic Acid (siNA) Molecules

As indicated above, the present disclosure provides siNA molecules comprising modified nucleotides. Any of the siNA molecules described herein may be double-stranded siNA (ds-siNA) molecules. The terms “siNA molecules” and “ds-siNA molecules” may be used interchangeably. In some embodiments, the ds-siNA molecules comprise a sense strand and an antisense strand.

The disclosed siNA molecules may comprise (a) at least one phosphorylation blocker, conjugated moiety, or 5′-stabilized end cap; and (b) a short interfering nucleic acid (siNA). In some embodiments, the phosphorylation blocker is a phosphorylation blocker disclosed herein. In some embodiments, the 5′-stabilized end cap is a 5′-stabilized end cap disclosed herein. The siNA may comprise any of the first nucleotide, second nucleotide, sense strand, or antisense strand sequences disclosed herein. The siNA may comprise 5 to 100, 5 to 90, 10 to 100, 10 to 90, 10 to 80, 10 to 70, 10 to 60, 10 to 50, 10 to 30, 10 to 25, 15 to 100, 15 to 90, 15 to 80, 15 to 70, 15 to 60, 15 to 50, 15 to 30, or 15 to 25 nucleotides. The siNA may comprise at least 5, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 nucleotides. The siNA may comprise less than or equal to 50, 45, 40, 39, 38, 37, 36, 35, 34, 33, 32, 31, 30, 29, 28, 27, 26, 25, 24, 23, 22, 21, 20, or 19 nucleotides. The nucleotides may be modified nucleotides. The siNA may be single stranded. The siNA may be double stranded. The siNA may comprise (a) a sense strand comprising 15 to 30, 15 to 25, 15 to 24, 15 to 23, 15 to 22, 15 to 21, 17 to 30, 17 to 25, 17 to 24, 17 to 23, 17 to 22, 17 to 21, 18 to 30, 18 to 25, 18 to 24, 18 to 23, 18 to 22, 18 to 21, 19 to 30, 19 to 25, 19 to 24, 19 to 23, 19 to 22, 19 to 21, 20 to 25, 20 to 24, 20 to 23, 21 to 25, 21 to 24, or 21 to 23 nucleotides; and (b) an antisense strand comprising 15 to 30, 15 to 25, 15 to 24, 15 to 23, 15 to 22, 15 to 21, 17 to 30, 17 to 25, 17 to 24, 17 to 23, 17 to 22, 17 to 21, 18 to 30, 18 to 25, 18 to 24, 18 to 23, 18 to 22, 18 to 21, 19 to 30, 19 to 25, 19 to 24, 19 to 23, 19 to 22, 19 to 21, 20 to 25, 20 to 24, 20 to 23, 21 to 25, 21 to 24, or 21 to 23 nucleotides. The siNA may comprise (a) a sense strand comprising about 15, 16, 17, 18, 19, 20, 21, 22, or 23 nucleotides; and (b) an antisense strand comprising about 15, 16, 17, 18, 19, 20, 21, 22, or 23 nucleotides. The siNA may comprise (a) a sense strand comprising about 19 nucleotides; and (b) an antisense strand comprising about 21 nucleotides. The siNA may comprise (a) a sense strand comprising about 21 nucleotides; and (b) an antisense strand comprising about 23 nucleotides.

In some embodiments, any of the siNA molecules disclosed herein further comprise one or more linkers independently selected from a phosphodiester (PO) linker, phosphorothioate (PS) linker, phosphorodithioate linker, and PS-mimic linker. In some embodiments, the PS-mimic linker is a sulfur linker. In some embodiments, the linkers are internucleotide linkers. Alternatively, or additionally, the linkers connect a nucleotide of the siNA molecule to at least one phosphorylation blocker, conjugated moiety, or 5′-stabilized end cap. In some embodiments, the linkers connect a conjugated moiety to a phosphorylation blocker or 5′-stabilized end cap.

Table 1 details sequences of the present disclosure useful for sense and antisense strands, disclosed in SEQ ID NOs: 1-2406 and 3393-4374. Table 2 details representative genome sequences of four pathogenic β-coronaviruses, disclosed in SEQ ID NOs: 2407-2410. It is understood that RNA sequences corresponding to these sequences constitute identical sequences with all T replaced with U.

In some embodiments, the target gene a sequence 15 to 30, 15 to 25, 15 to 23, 17 to 23, 19 to 23, or 19 to 21 nucleotides in length, and preferably 19 or 21 nucleotides in length, within a region of either two, three, or four of SEQ ID NOs: 2407, 2408, 2409, and 2410. In some embodiments, the first nucleotide sequence is identical to an RNA sequence corresponding to a region of each of SEQ ID NOs: 2407, 2408, 2409, and 2410. In some embodiments, the target gene a sequence 15 to 30, 15 to 25, 15 to 23, 17 to 23, 19 to 23, or 19 to 21 nucleotides in length, and preferably 19 or 21 nucleotides in length, within a region of either two, three, or four of the genomes of severe acute respiratory syndrome coronavirus 2 (SARS-CoV-2), human coronavirus OC43 (hCoV-OC43), Middle East respiratory syndrome-related coronavirus (MERS-CoV), and severe acute respiratory syndrome-related coronavirus (SARS-CoV). In some embodiments, the first nucleotide sequence is identical to an RNA sequence corresponding to a region of each of two, three, or four of the genomes of severe acute respiratory syndrome coronavirus 2 (SARS-CoV-2), human coronavirus OC43 (hCoV-OC43), Middle East respiratory syndrome-related coronavirus (MERS-CoV), and severe acute respiratory syndrome-related coronavirus (SARS-CoV). In some embodiments, the first nucleotide sequence is identical to the target gene. In some embodiments, the second nucleotide sequence is complementary to the target gene.

In some embodiments, the second nucleotide sequence is complementary to a sequence within a region of either two, three, or four of SEQ ID NOs: 2407, 2408, 2409, and 2410. In some embodiments, the second nucleotide sequence is complementary to an RNA sequence corresponding to a region of each of SEQ ID NOs: 2407, 2408, 2409, and 2410. In some embodiments, the second nucleotide sequence comprises a sequence corresponding to one of SEQ ID NOs: 1204-2406 and 3393-4374.

In some embodiments, the second nucleotide is complementary to a nucleotide region within SEQ ID NO: 2407, 2408, 2409, or 2410. In some embodiments, the second nucleotide sequence is complementary to 15 to 30, 15 to 25, 15 to 23, 15 to 22, 15 to 21, 17 to 25, 17 to 23, 17 to 22, 17 to 21, or 19 to 21 nucleotides, and preferably 19 to 21 nucleotides, and more preferably 19 or 21 nucleotides, within positions 190-216, 233-279, 288-324, 455-477, 626-651, 704-723, 3352-3378, 5384-5403, 6406-6483, 7532-7551, 9588-9606, 10484-10509, 11609-11630, 11834-11853, 12023-12045, 12212-12234, 12401-12420, 12839-12867, 12885-12924, 12966-12990, 13151-13176, 13363-13386, 13388-13416, 13458-13416, 13458-13520, 13762-13790, 14290-14312, 14404-14429, 14500-14531, 14623-14642, 14650-14687, 14698-14717, 14722-14748, 14750-14777, 14821-14846, 14854-14873, 14875-14903, 14962-14990, 14992-15020, 15055-15140, 15172-15200, 15310-15332, 15346-15367, 15496-15518, 15622-15644, 15838-15869, 15886-15905, 15985-16010, 16057-16079, 16186-16205, 16430-16448, 16822-16865, 16954-16976, 17008-17042, 17080-17111, 17137-17156, 17269-17289, 17530-17549, 17563-17582, 17680-17699, 17746-17765, 17857-17876, 17956-17975, 18100-18122, 18196-18218, 19618-19639, 19783-19802, 19831-19850, 20107-20130, 20776-20795, 21502-21524, 24302-24325, 24446-24465, 24620-24651, 24662-24684, 25034-25057, 25104-25128, 25364-25387, 25502-25530, 26191-26227, 26232-26267, 26269-26330, 26332-26394, 26450-26481, 26574-26600, 27003-27064, 27093-27111, 27183-27212, 27382-27407, 27511-27533, 27771-27818, 28270-28296, 28397-28434, 28513-28546, 28673-28692, 28706-28726, 28744-28794, 28799-28827, 28946-28972, 28976-29034, 29144-29172, 29174-29196, 29228-29259, 29285-29305, 29342-29394, 29444-29463, 29543-29566, 29598-29630, 29652-29687, 29689-29731, 29733-29757, or 29770-29828 of SEQ ID NO: 2407. In some embodiments, the second nucleotide sequence is complementary to any one of SEQ ID NOs: 1-1203 and 2411-3392. In some embodiments, the second nucleotide sequence is identical to an RNA corresponding to any one of SEQ ID NOs: 1204-2406 and 3393-4374.

In some embodiments, the first nucleotide sequence is identical to a nucleotide region within SEQ ID NOs: 2407, 2408, 2409, or 2410, with the exception that the thymines (Ts) in SEQ ID NOs: 2407, 2408, 2409, or 2410 are replaced with uracil (U). In some embodiments, the first nucleotide sequence is identical to 15 to 30, 15 to 25, 15 to 23, 15 to 22, 15 to 21, 17 to 25, 17 to 23, 17 to 22, 17 to 21, or 19 to 21 nucleotides, and preferably 19 to 21 nucleotides, and more preferably 19 or 21 nucleotides, within positions 190-216, 233-279, 288-324, 455-477, 626-651, 704-723, 3352-3378, 5384-5403, 6406-6483, 7532-7551, 9588-9606, 10484-10509, 11609-11630, 11834-11853, 12023-12045, 12212-12234, 12401-12420, 12839-12867, 12885-12924, 12966-12990, 13151-13176, 13363-13386, 13388-13416, 13458-13416, 13458-13520, 13762-13790, 14290-14312, 14404-14429, 14500-14531, 14623-14642, 14650-14687, 14698-14717, 14722-14748, 14750-14777, 14821-14846, 14854-14873, 14875-14903, 14962-14990, 14992-15020, 15055-15140, 15172-15200, 15310-15332, 15346-15367, 15496-15518, 15622-15644, 15838-15869, 15886-15905, 15985-16010, 16057-16079, 16186-16205, 16430-16448, 16822-16865, 16954-16976, 17008-17042, 17080-17111, 17137-17156, 17269-17289, 17530-17549, 17563-17582, 17680-17699, 17746-17765, 17857-17876, 17956-17975, 18100-18122, 18196-18218, 19618-19639, 19783-19802, 19831-19850, 20107-20130, 20776-20795, 21502-21524, 24302-24325, 24446-24465, 24620-24651, 24662-24684, 25034-25057, 25104-25128, 25364-25387, 25502-25530, 26191-26227, 26232-26267, 26269-26330, 26332-26394, 26450-26481, 26574-26600, 27003-27064, 27093-27111, 27183-27212, 27382-27407, 27511-27533, 27771-27818, 28270-28296, 28397-28434, 28513-28546, 28673-28692, 28706-28726, 28744-28794, 28799-28827, 28946-28972, 28976-29034, 29144-29172, 29174-29196, 29228-29259, 29285-29305, 29342-29394, 29444-29463, 29543-29566, 29598-29630, 29652-29687, 29689-29731, 29733-29757, or 29770-29828 of SEQ ID NO: 2407. In some embodiments, the first nucleotide sequence is identical to an RNA corresponding to any one of SEQ ID NOs: 1-1203 and 2411-3392. In some embodiments, the first nucleotide sequence is complementary to any one of SEQ ID NOs: 1204-2406 and 3393-4374.

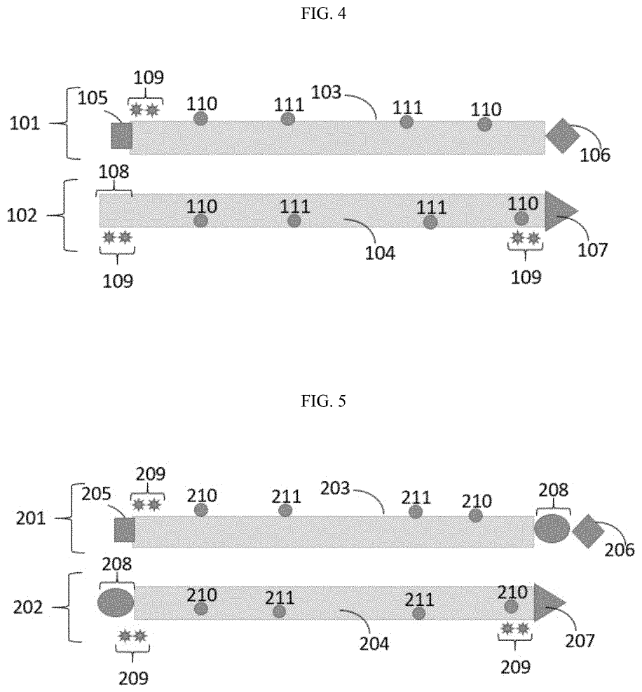

An exemplary siNA molecule of the present disclosure is shown in FIG. 4. As shown in FIG. 4, an exemplary siNA molecule can comprise a sense strand (101) and an antisense strand (102). The sense strand (101) may comprise a first oligonucleotide sequence (103). The first oligonucleotide sequence (103) may comprise one or more phosphorothioate internucleoside linkages (109). The phosphorothioate internucleoside linkage (109) may be between the nucleotides at the 5′ or 3′ terminal end of the first oligonucleotide sequence (103). The phosphorothioate internucleoside linkage (109) may be between the first three nucleotides from the 5′ end of the first oligonucleotide sequence (103). The first oligonucleotide sequence (103) may comprise one or more 2′-fluoro nucleotides (110). The first oligonucleotide sequence (103) may comprise one or more 2′-O-methyl nucleotides (111). The first oligonucleotide sequence (103) may comprise 15 or more modified nucleotides independently selected from 2′-fluoro nucleotides (110) and 2′-O-methyl nucleotides (111). The sense strand (101) may further comprise a phosphorylation blocker (105). The sense strand (101) may further comprise an optional conjugated moiety (106). The antisense strand (102) may comprise a second oligonucleotide sequence (104). The second oligonucleotide sequence (104) may comprise one or more phophorothioate internucleoside linkages (109). The phosphorothioate internucleoside linkage (109) may be between the nucleotides at the 5′ or 3′ terminal end of the second oligonucleotide sequence (104). The phosphorothioate internucleoside linkage (109) may be between the first three nucleotides from the 5′ end of the second oligonucleotide sequence (104). The phosphorothioate internucleoside linkage (109) may be between the first three nucleotides from the 3′ end of the second oligonucleotide sequence (104). The second oligonucleotide sequence (104) may comprise one or more 2′-fluoro nucleotides (110). The second oligonucleotide sequence (104) may comprise one or more 2′-O-methyl nucleotides (111). The second oligonucleotide sequence (104) may comprise 15 or more modified nucleotides independently selected from 2′-fluoro nucleotides (110) and 2′-O-methyl nucleotides (111). The antisense strand (102) may further comprise a 5′-stabilized end cap (107). The siNA may further comprise one or more blunt ends. Alternatively, or additionally, one end of the siNA may comprise an overhang (108). The overhang (108) may be part of the sense strand (101). The overhang (108) may be part of the antisense strand (102). The overhang (108) may be distinct from the first nucleotide sequence (103). The overhang (108) may be distinct from the second nucleotide sequence (104). The overhang (108) may be part of the first nucleotide sequence (103). The overhang (108) may be part of the second nucleotide sequence (104). The overhang (108) may comprise 1 or more nucleotides. The overhang (108) may comprise 1 or more deoxyribonucleotides. The overhang (108) may comprise 1 or more modified nucleotides. The overhang (108) may comprise 1 or more modified ribonucleotides. The sense strand (101) may be shorter than the antisense strand (102). The sense strand (101) may be the same length as the antisense strand (102). The sense strand (101) may be longer than the antisense strand (102).

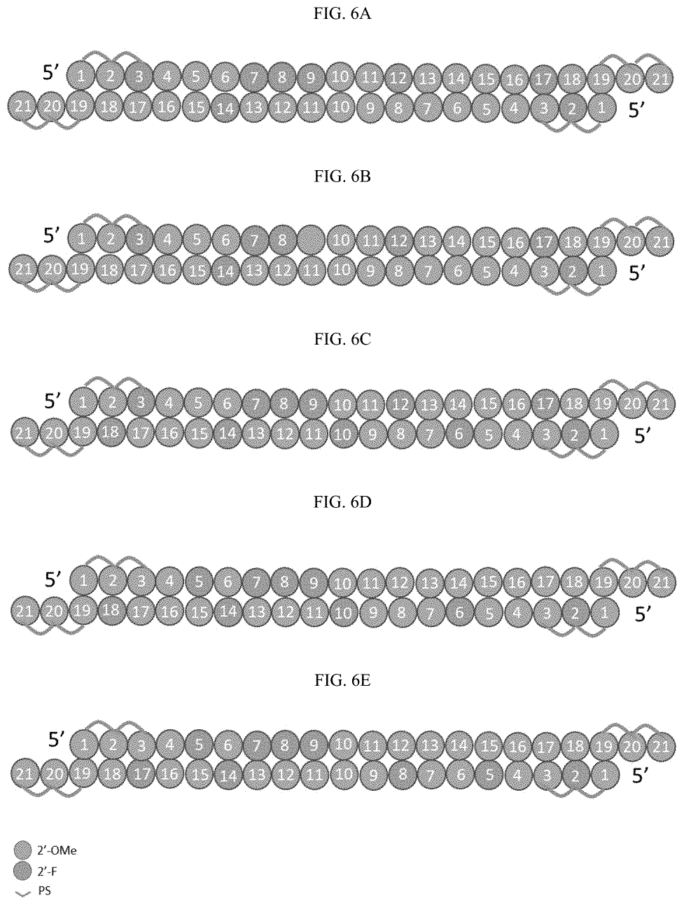

Another exemplary siNA molecule of the present disclosure is shown in FIG. 5. As shown in FIG. 5, an exemplary siNA molecule can comprise a sense strand (201) and an antisense strand (202). The sense strand (201) may comprise a first oligonucleotide sequence (203). The first oligonucleotide sequence (203) may comprise one or more phophorothioate internucleoside linkages (209). The phosphorothioate internucleoside linkage (209) may be between the nucleotides at the 5′ or 3′ terminal end of the first oligonucleotide sequence (203). The phosphorothioate internucleoside linkage (209) may be between the first three nucleotides from the 5′ end of the first oligonucleotide sequence (203). The first oligonucleotide sequence (203) may comprise one or more 2′-fluoro nucleotides (210). The first oligonucleotide sequence (203) may comprise one or more 2′-O-methyl nucleotides (211). The first oligonucleotide sequence (203) may comprise 15 or more modified nucleotides independently selected from 2′-fluoro nucleotides (210) and 2′-O-methyl nucleotides (211). The sense strand (201) may further comprise a phosphorylation blocker (205). The sense strand (201) may further comprise an optional conjugated moiety (206). The antisense strand (202) may comprise a second oligonucleotide sequence (204). The second oligonucleotide sequence (204) may comprise one or more phophorothioate internucleoside linkages (209). The phosphorothioate internucleoside linkage (209) may be between the nucleotides at the 5′ or 3′ terminal end of the second oligonucleotide sequence (204). The phosphorothioate internucleoside linkage (209) may be between the first three nucleotides from the 5′ end of the second oligonucleotide sequence (204). The phosphorothioate internucleoside linkage (209) may be between the first three nucleotides from the 3′ end of the second oligonucleotide sequence (204). The second oligonucleotide sequence (204) may comprise one or more 2′-fluoro nucleotides (210). The second oligonucleotide sequence (204) may comprise one or more 2′-O-methyl nucleotides (211). The second oligonucleotide sequence (204) may comprise 15 or more modified nucleotides independently selected from 2′-fluoro nucleotides (210) and 2′-O-methyl nucleotides (211). The antisense strand (202) may further comprise a 5′-stabilized end cap (207). The siNA may further comprise one or more overhangs (208). The overhang (208) may be part of the sense strand (201). The overhang (208) may be part of the antisense strand. (202). The overhang (208) may be distinct from the first nucleotide sequence (203). The overhang (208) may be distinct from the second nucleotide sequence (204). The overhang (208) may be part of the first nucleotide sequence (203). The overhang (208) may be part of the second nucleotide sequence (204). The overhang (208) may be adjacent to the 3′ end of the first nucleotide sequence (203). The overhang (208) may be adjacent to the 5′ end of the first nucleotide sequence (203). The overhang (208) may be adjacent to the 3′ end of the second nucleotide sequence (204). The overhang (208) may be adjacent to the 5′ end of the second nucleotide sequence (204). The overhang (208) may comprise 1 or more nucleotides. The overhang (208) may comprise 1 or more deoxyribonucleotides. The overhang (208) may comprise a TT sequence. The overhang (208) may comprise 1 or more modified nucleotides. The overhang (208) may comprise 1 or more modified nucleotides disclosed herein (e.g., 2-fluoro nucleotide, 2′-O-methyl nucleotide, 2′-fluoro nucleotide mimic, 2′-O-methyl nucleotide mimic, or a nucleotide comprising a modified nucleobase). The overhang (208) may comprise 1 or more modified ribonucleotides. The sense strand (201) may be shorter than the antisense strand (202). The sense strand (201) may be the same length as the antisense strand (202). The sense strand (201) may be longer than the antisense strand (202).

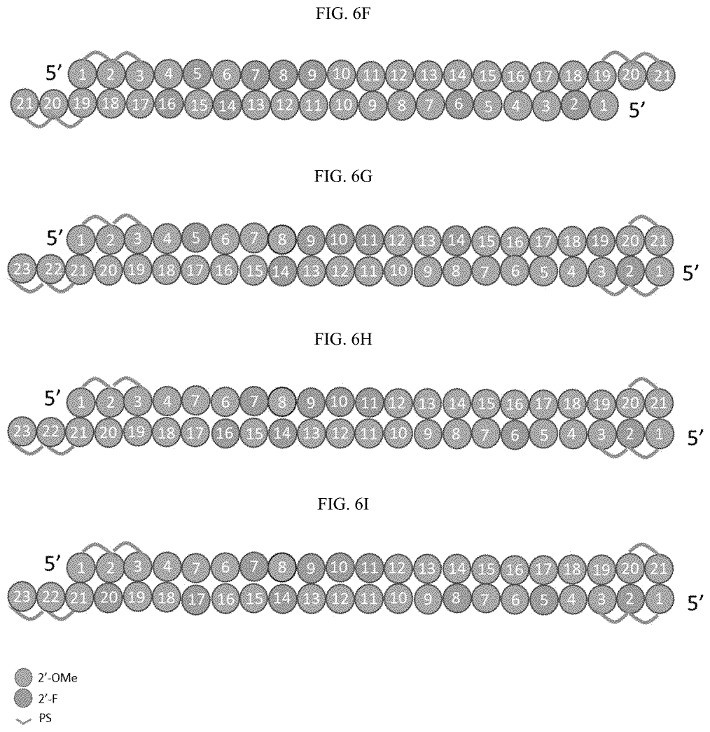

FIGS. 6A-6I depict exemplary ds-siNA modification patterns. As shown in FIGS. 6A-6G, an exemplary ds-siNA molecule may have the following formula:

5′-Aₙ¹Bₙ²Aₙ³Bₙ⁴Aₙ⁵Bₙ⁶Aₙ⁷Bₙ⁸Aₙ⁹-3′
3′-C_{q}¹A_{q}²B_{q}³A_{q}⁴B_{q}⁵A_{q}⁶B_{q}⁷A_{q}⁸B_{q}⁹A_{q}¹⁰B_{q}¹¹A_{q}¹²-5′

wherein:
the top strand is a sense strand comprising a first nucleotide sequence that is at least about 60%, 65%, 70%, 75%, 80%, 85%, 90%, 95%, or 100% identical to an RNA corresponding to a target gene, wherein the first nucleotide sequence comprises 15 to 30 nucleotides;
the bottom strand is an antisense strand comprising a second nucleotide sequence that is at least about 60%, 65%, 70%, 75%, 80%, 85%, 90%, 95%, or 100% complementary to the RNA corresponding to the target gene, wherein the second nucleotide sequence comprises 15 to 30 nucleotides;
each A is independently a 2′-O-methyl nucleotide or a nucleotide comprising a 5′ stabilized end cap or phosphorylation blocker;
B is a 2′-fluoro nucleotide;
C represents overhanging nucleotides and is a 2′-O-methyl nucleotide;
n¹=1-4 nucleotides in length;
each n², n⁶, n⁸, q³, q⁵, q⁷, q⁹, q¹¹, and q¹² is independently 0-1 nucleotides in length;
each n³ and n⁴ is independently 1-3 nucleotides in length;
n⁵ is 1-10 nucleotides in length;
n⁷ is 0-4 nucleotides in length;
each n⁹, q¹, and q² is independently 0-2 nucleotides in length;
q⁴ is 0-3 nucleotides in length;
q⁶ is 0-5 nucleotides in length;
q⁸ is 2-7 nucleotides in length; and
q¹⁰ is 2-11 nucleotides in length.

The ds-siNA may further comprise a conjugated moiety. The ds-siNA may further comprise (i) phosphorothioate internucleoside linkages between the nucleotides at positions 1 and 2 and positions 2 and 3 from the 5′ end of the sense strand; and (ii) phosphorothioate internucleoside linkages between the nucleotides at positions 1 and 2; positions 2 and 3; positions 19 and 20; and positions 20 and 21 from the 5′ end of the antisense strand. The ds-siNA may further comprise a 5′-stabilizing end cap. The 5′-stabilizing end cap may be a vinyl phosphonate. The 5′-stabilizing end cap may be attached to the 5′ end of the antisense strand. In some embodiments, the 2′-O-methyl nucleotide at position 1 from the 5′ end of the sense strand is further modified to contain a 5′ stabilizing end cap. In some embodiments, the 2′-O-methyl nucleotide at position 1 from the 5′ end of the antisense strand is further modified to contain a 5′ stabilizing end cap. In some embodiments, the 2′-O-methyl nucleotide at position 1 from the 5′ end of the sense strand is further modified to contain a phosphorylation blocker. In some embodiments, the 2′-O-methyl nucleotide at position 1 from the 3′ end of the sense strand is further modified to contain a phosphorylation blocker. In some embodiments, the 2′-O-methyl nucleotide at position 1 from the 5′ end of the antisense strand is further modified to contain a phosphorylation blocker. In some embodiments, the 2′-O-methyl nucleotide at position 1 from the 3′ end of the antisense strand is further modified to contain a phosphorylation blocker. An exemplary ds-siNA molecule may have the following formula:

5′-A₂₋₄B₁A₁₋₃B₂₋₃A₂₋₁₀B₀₋₁A₀₋₄B₀₋₁A₀₋₂-3′
3′-C₂A₀₋₂B₀₋₁A₀₋₃B₀₋₁A₀₋₅B₀₋₁A₂₋₇B₁A₂₋₁₁B₁A₁-5′

wherein:
the top strand is a sense strand comprising a first nucleotide sequence that is at least about 60%, 65%, 70%, 75%, 80%, 85%, 90%, 95%, or 100% identical to an RNA corresponding to a target gene, wherein the first nucleotide sequence comprises 15 to 30 nucleotides;
the bottom strand is an antisense strand comprising a second nucleotide sequence that is at least about 60%, 65%, 70%, 75%, 80%, 85%, 90%, 95%, or 100% complementary to the RNA corresponding to the target gene, wherein the second nucleotide sequence comprises 15 to 30 nucleotides;
each A is independently a 2′-O-methyl nucleotide or a nucleotide comprising a 5′ stabilized end cap or phosphorylation blocker;
B is a 2′-fluoro nucleotide;
C represents overhanging nucleotides and is a 2′-O-methyl nucleotide.

The ds-siNA may further comprise a conjugated moiety. The ds-siNA may further comprise (i) phosphorothioate internucleoside linkages between the nucleotides at positions 1 and 2 and positions 2 and 3 from the 5′ end of the sense strand; and (ii) phosphorothioate internucleoside linkages between the nucleotides at positions 1 and 2; positions 2 and 3; positions 19 and 20; and positions 20 and 21 from the 5′ end of the antisense strand. The ds-siNA may further comprise a 5′-stabilizing end cap. The 5′-stabilizing end cap may be a vinyl phosphonate. The vinyl phosphonate may be a deuterated vinyl phosphonate. The deuterated vinyl phosphonate may be a mono-deuterated vinyl phosphonate. The deuterated vinyl phosphonate may be a mono-di-deuterated vinyl phosphonate. The 5′-stabilizing end cap may be attached to the 5′ end of the antisense strand. The 5′-stabilizing end cap may be attached to the 3′ end of the antisense strand. The 5′-stabilizing end cap may be attached to the 5′ end of the sense strand. The 5′-stabilizing end cap may be attached to the 3′ end of the sense strand. In some embodiments, the 2′-O-methyl nucleotide at position 1 from the 5′ end of the sense strand is further modified to contain a 5′ stabilizing end cap. In some embodiments, the 2′-O-methyl nucleotide at position 1 from the 5′ end of the antisense strand is further modified to contain a 5′ stabilizing end cap. In some embodiments, the 2′-O-methyl nucleotide at position 1 from the 5′ end of the sense strand is further modified to contain a phosphorylation blocker. In some embodiments, the 2′-O-methyl nucleotide at position 1 from the 3′ end of the sense strand is further modified to contain a phosphorylation blocker. In some embodiments, the 2′-O-methyl nucleotide at position 1 from the 5′ end of the antisense strand is further modified to contain a phosphorylation blocker. In some embodiments, the 2′-O-methyl nucleotide at position 1 from the 3′ end of the antisense strand is further modified to contain a phosphorylation blocker.

The exemplary ds-siNA shown in FIGS. 6A-6I comprise (i) a sense strand comprising 19-21 nucleotides; and (ii) an antisense strand comprising 21-23 nucleotides. The ds-siNA may further comprise (iii) an optional conjugated moiety, wherein the conjugated moiety is attached to the 3′ end of the antisense strand and, in some embodiments, no ps would be needed at the 3′-end of the sense strand if it is conjugated to a moiety and such conjugation my also result in removal of the 5′ overhang on the sense strand. The ds-siNA may comprise a 2-nucleotide overhang consisting of nucleotides at positions 20 and 21 from the 5′ end of the antisense strand. The ds-siNA may comprise a 2-nucleotide overhang consisting of nucleotides at positions 22 and 23 from the 5′ end of the antisense strand. The ds-siNA may further comprise 1, 2, 3, 4, 5, 6 or more phosphorothioate (ps) internucleoside linkages. At least one phosphorothioate internucleoside linkage may be between the nucleotides at positions 1 and 2 or positions 2 and 3 from the 5′ end of the sense strand. At least one phosphorothioate internucleoside linkage may be between the nucleotides at positions 1 and 2 or positions 2 and 3 from the 5′ end of the antisense strand. At least one phosphorothioate internucleoside linkage may be between the nucleotides at positions 19 and 20, positions 20 and 21, positions 21 and 22, or positions 22 and 23 from the 5′ end of the antisense strand. As shown in FIGS. 6A-6G, 4-6 nucleotides in the sense strand may be 2′-fluoro nucleotides. As shown in FIGS. 6A-6G, 2-5 nucleotides in the antisense strand may be 2′-fluoro nucleotides. As shown in FIGS. 6A-6G, 13-15 nucleotides in the sense strand may be 2′-O-methyl nucleotides. As shown in FIGS. 6A-6G, 14-19 nucleotides in the antisense strand may be 2′-O-methyl nucleotides. As shown in FIGS. 6A-6G, the ds-siNA does not contain a base pair between 2′-fluoro nucleotides on the sense and antisense strands. In some embodiments, the 2′-O-methyl nucleotide at position 1 from the 5′ end of the sense strand is further modified to contain a 5′ stabilizing end cap. In some embodiments, the 2′-O-methyl nucleotide at position 1 from the 5′ end of the antisense strand is further modified to contain a 5′ stabilizing end cap. In some embodiments, the 2′-O-methyl nucleotide at position 1 from the 5′ end of the sense strand is further modified to contain a phosphorylation blocker. In some embodiments, the 2′-O-methyl nucleotide at position 1 from the 3′ end of the sense strand is further modified to contain a phosphorylation blocker. In some embodiments, the 2′-O-methyl nucleotide at position 1 from the 5′ end of the antisense strand is further modified to contain a phosphorylation blocker. In some embodiments, the 2′-O-methyl nucleotide at position 1 from the 3′ end of the antisense strand is further modified to contain a phosphorylation blocker.

As shown in FIG. 6A, a ds-siNA may comprise (a) a sense strand consisting of 21 nucleotides, wherein 2′-fluoro nucleotides are at positions 3, 7-9, 12, and 17 from the 5′ end of the sense strand, and wherein 2′-O-methyl nucleotides are at positions 1, 2, 4-6, 10, 11, 13-16, and 18-21 from the 5′ end of the sense strand; (b) an antisense strand consisting of 21 nucleotides, wherein nucleotides at positions 2 and 14 from the 5′ end of the antisense strand are 2′-fluoro nucleotides; and wherein nucleotides at positions 1, 3-13, and 15-21 are 2′-O-methyl nucleotides. The ds-siNA may further comprise a conjugated moiety attached to the 3′ end of the sense strand. The ds-siNA may further comprise (i) phosphorothioate internucleoside linkages between the nucleotides at positions 1 and 2; positions 2 and 3; positions 19 and 20; and positions 20 and 21 from the 5′ end of the sense strand; and (ii) phosphorothioate internucleoside linkages between the nucleotides at positions 1 and 2; positions 2 and 3; positions 19 and 20; and positions 20 and 21 from the 5′ end of the antisense strand. In some embodiments, the 2′-O-methyl nucleotide at position 1 from the 5′ end of the sense strand is further modified to contain a 5′ stabilizing end cap. In some embodiments, the 2′-O-methyl nucleotide at position 1 from the 5′ end of the antisense strand is further modified to contain a 5′ stabilizing end cap. In some embodiments, the 2′-O-methyl nucleotide at position 1 from the 5′ end of the sense strand is further modified to contain a phosphorylation blocker. In some embodiments, the 2′-O-methyl nucleotide at position 1 from the 3′ end of the sense strand is further modified to contain a phosphorylation blocker. In some embodiments, the 2′-O-methyl nucleotide at position 1 from the 5′ end of the antisense strand is further modified to contain a phosphorylation blocker. In some embodiments, the 2′-O-methyl nucleotide at position 1 from the 3′ end of the antisense strand is further modified to contain a phosphorylation blocker. In some embodiments, the 2′-O-methyl nucleotide at position 1 from the 5′ end of the sense strand is a d2vd3 nucleotide. In some embodiments, the 2′-O-methyl nucleotide at position 1 from the 5′ end of the antisense strand is a d2vd3 nucleotide. In some embodiments, the 2′-O-methyl nucleotide at position 1 from the 3′ end of the sense strand is a d2vd3 nucleotide. In some embodiments, the 2′-O-methyl nucleotide at position 1 from the 3′ end of the antisense strand is a d2vd3 nucleotide. In some embodiments, at least 1, 2, 3, 4 or more 2′-fluoro nucleotides on the sense strand or antisense strand is a 2′-fluoro nucleotide mimic. In some embodiments, at least 1, 2, 3, 4 or more 2′-fluoro nucleotides on the sense strand or antisense strand is a f4P, f2P, or fX nucleotide. In some embodiments, at least 1, 2, 3, 4 or more 2′-O-methyl nucleotide on the sense or antisense strand is a 2′-O-methyl nucleotide mimic.

As shown in FIG. 6B, a ds-siNA may comprise (a) a sense strand consisting of 21 nucleotides, wherein 2′-fluoro nucleotides are at positions 3, 7, 8, 12, and 17 from the 5′ end of the sense strand, and wherein 2′-O-methyl nucleotides are at positions 1, 2, 4-6, 9-11, 13-16, and 18-21 from the 5′ end of the sense strand; (b) an antisense strand consisting of 21 nucleotides, wherein nucleotides at positions 2 and 14 from the 5′ end of the antisense strand are 2′-fluoro nucleotides; and wherein nucleotides at positions 1, 3-13, and 15-21 are 2′-O-methyl nucleotides. The ds-siNA may further comprise a conjugated moiety attached to the 3′ end of the sense strand. The ds-siNA may further comprise (i) phosphorothioate internucleoside linkages between the nucleotides at positions 1 and 2; positions 2 and 3; positions 19 and 20; and positions 20 and 21 from the 5′ end of the sense strand; and (ii) phosphorothioate internucleoside linkages between the nucleotides at positions 1 and 2; positions 2 and 3; positions 19 and 20; and positions 20 and 21 from the 5′ end of the antisense strand. In some embodiments, the 2′-O-methyl nucleotide at position 1 from the 5′ end of the sense strand is further modified to contain a 5′ stabilizing end cap. In some embodiments, the 2′-O-methyl nucleotide at position 1 from the 5′ end of the antisense strand is further modified to contain a 5′ stabilizing end cap. In some embodiments, the 2′-O-methyl nucleotide at position 1 from the 5′ end of the sense strand is further modified to contain a phosphorylation blocker. In some embodiments, the 2′-O-methyl nucleotide at position 1 from the 3′ end of the sense strand is further modified to contain a phosphorylation blocker. In some embodiments, the 2′-O-methyl nucleotide at position 1 from the 5′ end of the antisense strand is further modified to contain a phosphorylation blocker. In some embodiments, the 2′-O-methyl nucleotide at position 1 from the 3′ end of the antisense strand is further modified to contain a phosphorylation blocker. In some embodiments, the 2′-O-methyl nucleotide at position 1 from the 5′ end of the sense strand is a d2vd3 nucleotide. In some embodiments, the 2′-O-methyl nucleotide at position 1 from the 5′ end of the antisense strand is a d2vd3 nucleotide. In some embodiments, the 2′-O-methyl nucleotide at position 1 from the 3′ end of the sense strand is a d2vd3 nucleotide. In some embodiments, the 2′-O-methyl nucleotide at position 1 from the 3′ end of the antisense strand is a d2vd3 nucleotide. In some embodiments, at least 1, 2, 3, 4 or more 2′-fluoro nucleotides on the sense strand or antisense strand is a 2′-fluoro nucleotide mimic. In some embodiments, at least 1, 2, 3, 4 or more 2′-fluoro nucleotides on the sense strand or antisense strand is a f4P, f2P, or fX nucleotide. In some embodiments, at least 1, 2, 3, 4 or more 2′-O-methyl nucleotide on the sense or antisense strand is a 2′-O-methyl nucleotide mimic.

As shown in FIG. 6C, a ds-siNA may comprise (a) a sense strand consisting of 21 nucleotides, wherein 2′-fluoro nucleotides are at positions 3, 7-9, 12, and 17 from the 5′ end of the sense strand, and wherein 2′-O-methyl nucleotides are at positions 1, 2, 4-6, 10, 11, 13-16, and 18-21 from the 5′ end of the sense strand; (b) an antisense strand consisting of 21 nucleotides, wherein the nucleotides in the antisense strand comprise an alternating 1:3 modification pattern, and wherein 1 nucleotide is a 2′-fluoro nucleotide and 3 nucleotides are 2′-O-methyl nucleotides. The ds-siNA may further comprise a conjugated moiety attached to the 3′ end of the sense strand. The ds-siNA may further comprise (i) phosphorothioate internucleoside linkages between the nucleotides at positions 1 and 2; and positions 2 and 3; positions 19 and 20; and positions 20 and 21 from the 5′ end of the sense strand; and (ii) phosphorothioate internucleoside linkages between the nucleotides at positions 1 and 2; positions 2 and 3; positions 19 and 20; and positions 20 and 21 from the 5′ end of the antisense strand. The ds-siNA may comprise 2-5 alternating 1:3 modification patterns on the antisense strand. In some embodiments, the 2′-O-methyl nucleotide at position 1 from the 5′ end of the sense strand is further modified to contain a 5′ stabilizing end cap. In some embodiments, the 2′-O-methyl nucleotide at position 1 from the 5′ end of the antisense strand is further modified to contain a 5′ stabilizing end cap. In some embodiments, the 2′-O-methyl nucleotide at position 1 from the 5′ end of the sense strand is further modified to contain a phosphorylation blocker. In some embodiments, the 2′-O-methyl nucleotide at position 1 from the 3′ end of the sense strand is further modified to contain a phosphorylation blocker. In some embodiments, the 2′-O-methyl nucleotide at position 1 from the 5′ end of the antisense strand is further modified to contain a phosphorylation blocker. In some embodiments, the 2′-O-methyl nucleotide at position 1 from the 3′ end of the antisense strand is further modified to contain a phosphorylation blocker. In some embodiments, the 2′-O-methyl nucleotide at position 1 from the 5′ end of the sense strand is a d2vd3 nucleotide. In some embodiments, the 2′-O-methyl nucleotide at position 1 from the 5′ end of the antisense strand is a d2vd3 nucleotide. In some embodiments, the 2′-O-methyl nucleotide at position 1 from the 3′ end of the sense strand is a d2vd3 nucleotide. In some embodiments, the 2′-O-methyl nucleotide at position 1 from the 3′ end of the antisense strand is a d2vd3 nucleotide. In some embodiments, at least 1, 2, 3, 4 or more 2′-fluoro nucleotides on the sense strand or antisense strand is a 2′-fluoro nucleotide mimic. In some embodiments, at least 1, 2, 3, 4 or more 2′-fluoro nucleotides on the sense strand is a f4P, f2P, or fX nucleotide. In some embodiments, at least 1, 2, 3, 4 or more 2′-fluoro nucleotides on the antisense strand is a f4P, f2P, or fX nucleotide. In some embodiments, at least 1, 2, 3, 4 or more 2′-O-methyl nucleotide on the sense or antisense strand is a 2′-O-methyl nucleotide mimic.

As shown in FIG. 6D, a ds-siNA may comprise (a) a sense strand consisting of 21 nucleotides, wherein 2′-fluoro nucleotides are at positions 5 and 7-9 from the 5′ end of the sense strand, and wherein 2′-O-methyl nucleotides are at positions 1-4, 6, and 10-21 from the 5′ end of the sense strand; (b) an antisense strand consisting of 21 nucleotides, wherein the nucleotides in the antisense strand comprise an alternating 1:3 modification pattern, and wherein 1 nucleotide is a 2′-fluoro nucleotide and 3 nucleotides are 2′-O-methyl nucleotides. The ds-siNA may further comprise a conjugated moiety attached to the 3′ end of the sense strand. The ds-siNA may further comprise (i) phosphorothioate internucleoside linkages between the nucleotides at positions 1 and 2; positions 2 and 3; positions 19 and 20; and positions 20 and 21 from the 5′ end of the sense strand; and (ii) phosphorothioate internucleoside linkages between the nucleotides at positions 1 and 2; positions 2 and 3; positions 19 and 20; and positions 20 and 21 from the 5′ end of the antisense strand. The ds-siNA may comprise 2-5 alternating 1:3 modification patterns on the antisense strand. The alternating 1:3 modification pattern may start at the nucleotide at any of positions 2, 6, 10, 14, and/or 18 from the 5′ end of the antisense strand. In some embodiments, the 2′-O-methyl nucleotide at position 1 from the 5′ end of the sense strand is further modified to contain a 5′ stabilizing end cap. In some embodiments, the 2′-O-methyl nucleotide at position 1 from the 5′ end of the antisense strand is further modified to contain a 5′ stabilizing end cap. In some embodiments, the 2′-O-methyl nucleotide at position 1 from the 5′ end of the sense strand is further modified to contain a phosphorylation blocker. In some embodiments, the 2′-O-methyl nucleotide at position 1 from the 3′ end of the sense strand is further modified to contain a phosphorylation blocker. In some embodiments, the 2′-O-methyl nucleotide at position 1 from the 5′ end of the antisense strand is further modified to contain a phosphorylation blocker. In some embodiments, the 2′-O-methyl nucleotide at position 1 from the 3′ end of the antisense strand is further modified to contain a phosphorylation blocker. In some embodiments, the 2′-O-methyl nucleotide at position 1 from the 5′ end of the sense strand is a d2vd3 nucleotide. In some embodiments, the 2′-O-methyl nucleotide at position 1 from the 5′ end of the antisense strand is a d2vd3 nucleotide. In some embodiments, the 2′-O-methyl nucleotide at position 1 from the 3′ end of the sense strand is a d2vd3 nucleotide. In some embodiments, the 2′-O-methyl nucleotide at position 1 from the 3′ end of the antisense strand is a d2vd3 nucleotide. In some embodiments, at least 1, 2, 3, 4 or more 2′-fluoro nucleotides on the sense strand or antisense strand is a 2′-fluoro nucleotide mimic. In some embodiments, at least 1, 2, 3, 4 or more 2′-fluoro nucleotides on the sense strand is a f4P, f2P, or fX nucleotide. In some embodiments, at least 1, 2, 3, 4 or more 2′-fluoro nucleotides on the antisense strand is a f4P, f2P, or fX nucleotide. In some embodiments, at least 1, 2, 3, 4 or more 2′-O-methyl nucleotide on the sense or antisense strand is a 2′-O-methyl nucleotide mimic.

As shown in FIG. 6E, a ds-siNA may comprise (a) a sense strand consisting of 21 nucleotides, wherein 2′-fluoro nucleotides are at positions 5 and 7-9 from the 5′ end of the sense strand, and wherein 2′-O-methyl nucleotides are at positions 1-4, 6, and 10-21 from the 5′ end of the sense strand; (b) an antisense strand consisting of 21 nucleotides, wherein nucleotides at positions 2, 5, 8, 14, and 17 from the 5′ end of the antisense strand are 2′-fluoro nucleotides; and wherein nucleotides at positions 1, 3-13, and 15-21 are 2′-O-methyl nucleotides. The ds-siNA may further comprise a conjugated moiety attached to the 3′ end of the sense strand. The ds-siNA may further comprise (i) phosphorothioate internucleoside linkages between the nucleotides at positions 1 and 2; positions 2 and 3; positions 19 and 20; and positions 20 and 21 from the 5′ end of the sense strand; and (ii) phosphorothioate internucleoside linkages between the nucleotides at positions 1 and 2; positions 2 and 3; positions 19 and 20; and positions 20 and 21 from the 5′ end of the antisense strand. The ds-siNA may comprise 2-5 alternating 1:2 modification patterns on the antisense strand. The alternating 1:2 modification pattern may start at the nucleotide at any of positions 2, 5, 8, 14, and/or 17 from the 5′ end of the antisense strand. In some embodiments, the ds-siNA comprises (a) a sense strand consisting of 19 nucleotides, wherein 2′-fluoro nucleotides are at positions 5 and 7-9 from the 5′ end of the sense strand, and wherein 2′-O-methyl nucleotides are at positions 1-4, 6, and 10-19 from the 5′ end of the sense strand; (b) an antisense strand consisting of 21 nucleotides, wherein 2′-fluoro nucleotides are at positions 2, 5, 8, 14, and 17 from the 5′ end of the antisense strand, and wherein 2′-O-methyl nucleotides are at positions 1, 3, 4, 6, 7, 9-13, 15, 16, and 18-21 from the 5′ end of the sense strand. In some embodiments, the 2′-O-methyl nucleotide at position 1 from the 5′ end of the sense strand is further modified to contain a 5′ stabilizing end cap. In some embodiments, the 2′-O-methyl nucleotide at position 1 from the 5′ end of the antisense strand is further modified to contain a 5′ stabilizing end cap. In some embodiments, the 2′-O-methyl nucleotide at position 1 from the 5′ end of the sense strand is further modified to contain a phosphorylation blocker. In some embodiments, the 2′-O-methyl nucleotide at position 1 from the 3′ end of the sense strand is further modified to contain a phosphorylation blocker. In some embodiments, the 2′-O-methyl nucleotide at position 1 from the 5′ end of the antisense strand is further modified to contain a phosphorylation blocker. In some embodiments, the 2′-O-methyl nucleotide at position 1 from the 3′ end of the antisense strand is further modified to contain a phosphorylation blocker. In some embodiments, the 2′-O-methyl nucleotide at position 1 from the 5′ end of the sense strand is a d2vd3 nucleotide. In some embodiments, the 2′-O-methyl nucleotide at position 1 from the 5′ end of the antisense strand is a d2vd3 nucleotide. In some embodiments, the 2′-O-methyl nucleotide at position 1 from the 3′ end of the sense strand is a d2vd3 nucleotide. In some embodiments, the 2′-O-methyl nucleotide at position 1 from the 3′ end of the antisense strand is a d2vd3 nucleotide. In some embodiments, at least 1, 2, 3, 4 or more 2′-fluoro nucleotides on the sense strand or antisense strand is a 2′-fluoro nucleotide mimic. In some embodiments, at least 1, 2, 3, 4 or more 2′-fluoro nucleotides on the sense strand is a f4P, f2P, or fX nucleotide. In some embodiments, at least 1, 2, 3, 4 or more 2′-fluoro nucleotides on the antisense strand is a f4P, f2P, or fX nucleotide. In some embodiments, at least 1, 2, 3, 4 or more 2′-O-methyl nucleotide on the sense or antisense strand is a 2′-O-methyl nucleotide mimic.

As shown in FIG. 6F, a ds-siNA may comprise (a) a sense strand consisting of 21 nucleotides, wherein 2′-fluoro nucleotides are at positions 5 and 7-9 from the 5′ end of the sense strand, and wherein 2′-O-methyl nucleotides are at positions 1-4, 6, and 10-21 from the 5′ end of the sense strand; (b) an antisense strand consisting of 21 nucleotides, wherein 2′-fluoro nucleotides are at positions 2, 6, 14, and 16 from the 5′ end of the antisense strand, and wherein 2′-O-methyl nucleotides are at positions 1, 3-5, 7-13, 15, and 17-21 from the 5′ end of the antisense strand. The ds-siNA may further comprise a conjugated moiety attached to the 3′ end of the sense strand. The ds-siNA may further comprise (i) phosphorothioate internucleoside linkages between the nucleotides at positions 1 and 2; positions 2 and 3; positions 19 and 20; and positions 20 and 21 from the 5′ end of the sense strand; and (ii) phosphorothioate internucleoside linkages between the nucleotides at positions 1 and 2; positions 2 and 3; positions 19 and 20; and positions 20 and 21 from the 5′ end of the antisense strand. In some embodiments, at least 1, 2, 3, 4 or more 2′-fluoro nucleotides on the sense strand or antisense strand is a f4P, f2P, or fX nucleotide. In some embodiments, at least 1, 2, 3, 4 or more 2′-fluoro nucleotides on the sense strand or antisense strand is a f4P nucleotide. In some embodiments, at least 1, 2, 3, or 4 of the 2′-fluoro-nucleotides at positions 2, 6, 14, and 16 from the 5′ end of the antisense strand is a f4P nucleotide. In some embodiments, at least one of the 2′-fluoro-nucleotides at positions 2, 6, 14, and 16 from the 5′ end of the antisense strand is a f4P nucleotide. In some embodiments, at least two of the 2′-fluoro-nucleotides at positions 2, 6, 14, and 16 from the 5′ end of the antisense strand is a f4P nucleotide. In some embodiments, less than or equal to 3 of the 2′-fluoro-nucleotides at positions 2, 6, 14, and 16 from the 5′ end of the antisense strand is a f4P nucleotide. In some embodiments, less than or equal to 2 of the 2′-fluoro-nucleotides at positions 2, 6, 14, and 16 from the 5′ end of the antisense strand is a f4P nucleotide. In some embodiments, the 2′-fluoro-nucleotide at position 2 from the 5′ end of the antisense strand is a f4P nucleotide. In some embodiments, the 2′-fluoro-nucleotide at position 6 from the 5′ end of the antisense strand is a f4P nucleotide. In some embodiments, the 2′-fluoro-nucleotide at position 14 from the 5′ end of the antisense strand is a f4P nucleotide. In some embodiments, the 2′-fluoro-nucleotide at position 16 from the 5′ end of the antisense strand is a f4P nucleotide. In some embodiments, at least 1, 2, 3, 4 or more 2′-fluoro nucleotides on the sense strand or antisense strand is a f2P nucleotide. In some embodiments, at least 1, 2, 3, or 4 of the 2′-fluoro-nucleotides at positions 2, 6, 14, and 16 from the 5′ end of the antisense strand is a f2P nucleotide. In some embodiments, at least one of the 2′-fluoro-nucleotides at positions 2, 6, 14, and 16 from the 5′ end of the antisense strand is a f2P nucleotide. In some embodiments, at least two of the 2′-fluoro-nucleotides at positions 2, 6, 14, and 16 from the 5′ end of the antisense strand is a f2P nucleotide. In some embodiments, less than or equal to 3 of the 2′-fluoro-nucleotides at positions 2, 6, 14, and 16 from the 5′ end of the antisense strand is a f2P nucleotide. In some embodiments, less than or equal to 2 of the 2′-fluoro-nucleotides at positions 2, 6, 14, and 16 from the 5′ end of the antisense strand is a f2P nucleotide. In some embodiments, the 2′-fluoro-nucleotide at position 2 from the 5′ end of the antisense strand is a f2P nucleotide. In some embodiments, the 2′-fluoro-nucleotide at position 6 from the 5′ end of the antisense strand is a f2P nucleotide. In some embodiments, the 2′-fluoro-nucleotide at position 14 from the 5′ end of the antisense strand is a f2P nucleotide. In some embodiments, the 2′-fluoro-nucleotide at position 16 from the 5′ end of the antisense strand is a f2P nucleotide. In some embodiments, at least 1, 2, 3, 4 or more 2′-fluoro nucleotides on the sense strand or antisense strand is a fX nucleotide. In some embodiments, at least 1, 2, 3, or 4 of the 2′-fluoro-nucleotides at positions 2, 6, 14, and 16 from the 5′ end of the antisense strand is a fX nucleotide. In some embodiments, at least one of the 2′-fluoro-nucleotides at positions 2, 6, 14, and 16 from the 5′ end of the antisense strand is a fX nucleotide. In some embodiments, at least two of the 2′-fluoro-nucleotides at positions 2, 6, 14, and 16 from the 5′ end of the antisense strand is a fX nucleotide. In some embodiments, less than or equal to 3 of the 2′-fluoro-nucleotides at positions 2, 6, 14, and 16 from the 5′ end of the antisense strand is a fX nucleotide. In some embodiments, less than or equal to 2 of the 2′-fluoro-nucleotides at positions 2, 6, 14, and 16 from the 5′ end of the antisense strand is a fX nucleotide. In some embodiments, the 2′-fluoro-nucleotide at position 2 from the 5′ end of the antisense strand is a fX nucleotide. In some embodiments, the 2′-fluoro-nucleotide at position 6 from the 5′ end of the antisense strand is a fX nucleotide. In some embodiments, the 2′-fluoro-nucleotide at position 14 from the 5′ end of the antisense strand is a fX nucleotide. In some embodiments, the 2′-fluoro-nucleotide at position 16 from the 5′ end of the antisense strand is a fX nucleotide. In some embodiments, the 2′-O-methyl nucleotide at position 1 from the 5′ end of the sense strand is further modified to contain a 5′ stabilizing end cap. In some embodiments, the 2′-O-methyl nucleotide at position 1 from the 5′ end of the antisense strand is further modified to contain a 5′ stabilizing end cap. In some embodiments, the 2′-O-methyl nucleotide at position 1 from the 5′ end of the sense strand is further modified to contain a phosphorylation blocker. In some embodiments, the 2′-O-methyl nucleotide at position 1 from the 3′ end of the sense strand is further modified to contain a phosphorylation blocker. In some embodiments, the 2′-O-methyl nucleotide at position 1 from the 5′ end of the antisense strand is further modified to contain a phosphorylation blocker. In some embodiments, the 2′-O-methyl nucleotide at position 1 from the 3′ end of the antisense strand is further modified to contain a phosphorylation blocker. In some embodiments, the 2′-O-methyl nucleotide at position 1 from the 5′ end of the sense strand is a d2vd3 nucleotide. In some embodiments, the 2′-O-methyl nucleotide at position 1 from the 5′ end of the antisense strand is a d2vd3 nucleotide. In some embodiments, the 2′-O-methyl nucleotide at position 1 from the 3′ end of the sense strand is a d2vd3 nucleotide. In some embodiments, the 2′-O-methyl nucleotide at position 1 from the 3′ end of the antisense strand is a d2vd3 nucleotide. In some embodiments, at least 1, 2, 3, 4 or more 2′-fluoro nucleotides on the sense strand or antisense strand is a 2′-fluoro nucleotide mimic. In some embodiments, at least 1, 2, 3, 4 or more 2′-fluoro nucleotides on the sense strand is a f4P, f2P, or fX nucleotide. In some embodiments, at least 1, 2, 3, 4 or more 2′-fluoro nucleotides on the antisense strand is a f4P, f2P, or fX nucleotide. In some embodiments, at least 1, 2, 3, 4 or more 2′-O-methyl nucleotide on the sense or antisense strand is a 2′-O-methyl nucleotide mimic.

As shown in FIG. 6G, a ds-siNA may comprise (a) a sense strand consisting of 21 nucleotides, wherein 2′-fluoro nucleotides are at positions 5, 9-11, 14, and 19 from the 5′ end of the sense strand, and wherein 2′-O-methyl nucleotides are at positions 1-4, 6-8, 12, 13, 15-18, 20, and 21 from the 5′ end of the sense strand; and (b) an antisense strand consisting of 23 nucleotides, wherein 2′-flouro nucleotides are at positions 2 and 14 from the 5′ end of the antisense strand, and wherein 2′-O-methyl nucleotides are at positions 1, 3-13, and 15-23 from the 5′ end of the antisense strand. The ds-siNA may further comprise a conjugated moiety attached to the 3′ end of the sense strand. The ds-siNA may further comprise (i) phosphorothioate internucleoside linkages between the nucleotides at positions 1 and 2; positions 2 and 3; and positions 20 and 21 from the 5′ end of the sense strand; and (ii) phosphorothioate internucleoside linkages between the nucleotides at positions 1 and 2; positions 2 and 3; positions 21 and 22; and positions 22 and 23 from the 5′ end of the antisense strand. In some embodiments, the 2′-O-methyl nucleotide at position 1 from the 5′ end of the sense strand is further modified to contain a 5′ stabilizing end cap. In some embodiments, the 2′-O-methyl nucleotide at position 1 from the 5′ end of the antisense strand is further modified to contain a 5′ stabilizing end cap. In some embodiments, the 2′-O-methyl nucleotide at position 1 from the 5′ end of the sense strand is further modified to contain a phosphorylation blocker. In some embodiments, the 2′-O-methyl nucleotide at position 1 from the 3′ end of the sense strand is further modified to contain a phosphorylation blocker. In some embodiments, the 2′-O-methyl nucleotide at position 1 from the 5′ end of the antisense strand is further modified to contain a phosphorylation blocker. In some embodiments, the 2′-O-methyl nucleotide at position 1 from the 3′ end of the antisense strand is further modified to contain a phosphorylation blocker. In some embodiments, the 2′-O-methyl nucleotide at position 1 from the 5′ end of the sense strand is a d2vd3 nucleotide. In some embodiments, the 2′-O-methyl nucleotide at position 1 from the 5′ end of the antisense strand is a d2vd3 nucleotide. In some embodiments, the 2′-O-methyl nucleotide at position 1 from the 3′ end of the sense strand is a d2vd3 nucleotide. In some embodiments, the 2′-O-methyl nucleotide at position 1 from the 3′ end of the antisense strand is a d2vd3 nucleotide. In some embodiments, at least 1, 2, 3, 4 or more 2′-fluoro nucleotides on the sense strand or antisense strand is a 2′-fluoro nucleotide mimic. In some embodiments, at least 1, 2, 3, 4 or more 2′-fluoro nucleotides on the sense strand is a f4P, f2P, or fX nucleotide. In some embodiments, at least 1, 2, 3, 4 or more 2′-fluoro nucleotides on the antisense strand is a f4P, f2P, or fX nucleotide. In some embodiments, at least 1, 2, 3, 4 or more 2′-O-methyl nucleotide on the sense or antisense strand is a 2′-O-methyl nucleotide mimic.

As shown in FIG. 6H, a ds-siNA may comprise (a) a sense strand consisting of 21 nucleotides, wherein 2′-fluoro nucleotides are at positions 7 and 9-11 from the 5′ end of the sense strand, and wherein 2′-O-methyl nucleotides are at positions 1-6, 8, and 12-21 from the 5′ end of the sense strand; and (b) an antisense strand consisting of 23 nucleotides, wherein 2′-flouro nucleotides are at positions 2, 6, 14, and 16 from the 5′ end of the antisense strand, and wherein 2′-O-methyl nucleotides are at positions 1, 3-5, 7-13, 15, and 17-23 from the 5′ end of the antisense strand. The ds-siNA may further comprise a conjugated moiety attached to the 3′ end of the sense strand. The ds-siNA may further comprise (i) phosphorothioate internucleoside linkages between the nucleotides at positions 1 and 2; positions 2 and 3; and positions 20 and 21 from the 5′ end of the sense strand; and (ii) phosphorothioate internucleoside linkages between the nucleotides at positions 1 and 2; positions 2 and 3; positions 21 and 22; and positions 22 and 23 from the 5′ end of the antisense strand. In some embodiments, the 2′-O-methyl nucleotide at position 1 from the 5′ end of the sense strand is further modified to contain a 5′ stabilizing end cap. In some embodiments, the 2′-O-methyl nucleotide at position 1 from the 5′ end of the antisense strand is further modified to contain a 5′ stabilizing end cap. In some embodiments, the 2′-O-methyl nucleotide at position 1 from the 5′ end of the sense strand is further modified to contain a phosphorylation blocker. In some embodiments, the 2′-O-methyl nucleotide at position 1 from the 3′ end of the sense strand is further modified to contain a phosphorylation blocker. In some embodiments, the 2′-O-methyl nucleotide at position 1 from the 5′ end of the antisense strand is further modified to contain a phosphorylation blocker. In some embodiments, the 2′-O-methyl nucleotide at position 1 from the 3′ end of the antisense strand is further modified to contain a phosphorylation blocker. In some embodiments, the 2′-O-methyl nucleotide at position 1 from the 5′ end of the sense strand is a d2vd3 nucleotide. In some embodiments, the 2′-O-methyl nucleotide at position 1 from the 5′ end of the antisense strand is a d2vd3 nucleotide. In some embodiments, the 2′-O-methyl nucleotide at position 1 from the 3′ end of the sense strand is a d2vd3 nucleotide. In some embodiments, the 2′-O-methyl nucleotide at position 1 from the 3′ end of the antisense strand is a d2vd3 nucleotide. In some embodiments, at least 1, 2, 3, 4 or more 2′-fluoro nucleotides on the sense strand or antisense strand is a 2′-fluoro nucleotide mimic. In some embodiments, at least 1, 2, 3, 4 or more 2′-fluoro nucleotides on the sense strand is a f4P, f2P, or fX nucleotide. In some embodiments, at least 1, 2, 3, 4 or more 2′-fluoro nucleotides on the antisense strand is a f4P, f2P, or fX nucleotide. In some embodiments, at least 1, 2, 3, 4 or more 2′-O-methyl nucleotide on the sense or antisense strand is a 2′-O-methyl nucleotide mimic.

As shown in FIG. 6I, a ds-siNA may comprise (a) a sense strand consisting of 21 nucleotides, wherein 2′-fluoro nucleotides are at positions 7 and 9-11 from the 5′ end of the sense strand, and wherein 2′-O-methyl nucleotides are at positions 1-6, 8, and 12-21 from the 5′ end of the sense strand; and (b) an antisense strand consisting of 23 nucleotides, wherein 2′-flouro nucleotides are at positions 2, 5, 8, 14, 17, and 20 from the 5′ end of the antisense strand, and wherein 2′-O-methyl nucleotides are at positions 1, 3, 4, 6, 9-13, 15, 16, 18, 19, and 21-23 from the 5′ end of the antisense strand. The ds-siNA may further comprise a conjugated moiety attached to the 3′ end of the sense strand. The ds-siNA may further comprise (i) phosphorothioate internucleoside linkages between the nucleotides at positions 1 and 2; positions 2 and 3; and positions 20 and 21 from the 5′ end of the sense strand; and (ii) phosphorothioate internucleoside linkages between the nucleotides at positions 1 and 2; positions 2 and 3; positions 21 and 22; and positions 22 and 23 from the 5′ end of the antisense strand. In some embodiments, the 2′-O-methyl nucleotide at position 1 from the 5′ end of the sense strand is further modified to contain a 5′ stabilizing end cap. In some embodiments, the 2′-O-methyl nucleotide at position 1 from the 5′ end of the antisense strand is further modified to contain a 5′ stabilizing end cap. In some embodiments, the 2′-O-methyl nucleotide at position 1 from the 5′ end of the sense strand is further modified to contain a phosphorylation blocker. In some embodiments, the 2′-O-methyl nucleotide at position 1 from the 3′ end of the sense strand is further modified to contain a phosphorylation blocker. In some embodiments, the 2′-O-methyl nucleotide at position 1 from the 5′ end of the antisense strand is further modified to contain a phosphorylation blocker. In some embodiments, the 2′-O-methyl nucleotide at position 1 from the 3′ end of the antisense strand is further modified to contain a phosphorylation blocker. In some embodiments, the 2′-O-methyl nucleotide at position 1 from the 5′ end of the sense strand is a d2vd3 nucleotide. In some embodiments, the 2′-O-methyl nucleotide at position 1 from the 5′ end of the antisense strand is a d2vd3 nucleotide. In some embodiments, the 2′-O-methyl nucleotide at position 1 from the 3′ end of the sense strand is a d2vd3 nucleotide. In some embodiments, the 2′-O-methyl nucleotide at position 1 from the 3′ end of the antisense strand is a d2vd3 nucleotide. In some embodiments, at least 1, 2, 3, 4 or more 2′-fluoro nucleotides on the sense strand or antisense strand is a 2′-fluoro nucleotide mimic. In some embodiments, at least 1, 2, 3, 4 or more 2′-fluoro nucleotides on the sense strand is a f4P, f2P, or fX nucleotide. In some embodiments, at least 1, 2, 3, 4 or more 2′-fluoro nucleotides on the antisense strand is a f4P, f2P, or fX nucleotide. In some embodiments, at least 1, 2, 3, 4 or more 2′-O-methyl nucleotide on the sense or antisense strand is a 2′-O-methyl nucleotide mimic.

In some embodiments, the nucleotides in the antisense strand may comprise an alternating 1:2 modification pattern, wherein 1 nucleotide is a 2′-fluoro nucleotide and 2 nucleotides are 2′-O-methyl nucleotides. In some embodiments, the nucleotides in the antisense strand may comprise an alternating 1:1 modification pattern (i.e., an alternating pattern), wherein 1 nucleotide is a 2′-fluoro nucleotide and 1 nucleotide is a 2′-O-methyl nucleotide in an alternating fashion. These alternating modification patterns may start at any nucleotide of the antisense strand.

Any of the siNAs disclosed herein may comprise a sense strand and an antisense strand. The sense strand may comprise a first nucleotide sequence that is 15 to 30 nucleotides in length. The antisense strand may comprise a second nucleotide sequence that is 15 to 30 nucleotides in length.

A double-stranded short interfering nucleic acid (ds-siNA) molecule of this disclosure may comprise: (a) a sense strand comprising a first nucleotide sequence that is at least about 60%, 65%, 70%, 75%, 80%, 85%, 90%, 95%, or 100% identical to an RNA corresponding to a target gene, wherein the first nucleotide sequence: (i) is 15 to 30 nucleotides in length; and (ii) comprises 15 or more modified nucleotides independently selected from a 2′-O-methyl nucleotide and a 2′-fluoro nucleotide, wherein at least one modified nucleotide is a 2′-O-methyl nucleotide and the nucleotide at position 3, 5, 7, 8, 9, 10, 11, 12, 14, 17, and/or 19 from the 5′ end of the first nucleotide sequence is a 2′-fluoro nucleotide; and (b) an antisense strand comprising a second nucleotide sequence that is at least about 60%, 65%, 70%, 75%, 80%, 85%, 90%, 95%, or 100% complementary to the RNA corresponding to the target gene, wherein the second nucleotide sequence: (i) is 15 to 30 nucleotides in length; and (ii) comprises 15 or more modified nucleotides independently selected from a 2′-O-methyl nucleotide and a 2′-fluoro nucleotide, wherein at least one modified nucleotide is a 2′-O-methyl nucleotide and at least one modified nucleotide is a 2′-fluoro nucleotide.

A double-stranded short interfering nucleic acid (ds-siNA) molecule of the disclosure may comprise: (a) a sense strand comprising a first nucleotide sequence that is at least about 60%, 65%, 70%, 75%, 80%, 85%, 90%, 95%, or 100% identical to an RNA corresponding to a target gene, wherein the first nucleotide sequence: (i) is 15 to 30 nucleotides in length; and (ii) comprises 15 or more modified nucleotides independently selected from a 2′-O-methyl nucleotide and a 2′-fluoro nucleotide, wherein at least one modified nucleotide is a 2′-O-methyl nucleotide and the nucleotide at position 3, 5, 7, 8, 9, 10, 11, 12, 14, 17, and/or 19 from the 5′ end of the first nucleotide sequence is a 2′-fluoro nucleotide; and (b) an antisense strand comprising a second nucleotide sequence that is at least about 60%, 65%, 70%, 75%, 80%, 85%, 90%, 95%, or 100% complementary to the RNA corresponding to the target gene, wherein the second nucleotide sequence: (i) is 15 to 30 nucleotides in length; and (ii) comprises 15 or more modified nucleotides independently selected from a 2′-O-methyl nucleotide and a 2′-fluoro nucleotide, wherein at least one modified nucleotide is a 2′-O-methyl nucleotide and at least one modified nucleotide is a 2′-fluoro nucleotide.

A double-stranded short interfering nucleic acid (ds-siNA) molecule of the disclosure may comprise: (a) a sense strand comprising a first nucleotide sequence that is at least about 60%, 65%, 70%, 75%, 80%, 85%, 90%, 95%, or 100% identical to an RNA corresponding to a target gene, wherein the first nucleotide sequence: (i) is 15 to 30 nucleotides in length; and (ii) comprises 15 or more modified nucleotides independently selected from a 2′-O-methyl nucleotide and a 2′-fluoro nucleotide, wherein at least one modified nucleotide is a 2′-O-methyl nucleotide and the nucleotide at position 7 from the 5′ end of the first nucleotide sequence is a 2′-fluoro nucleotide; and (b) an antisense strand comprising a second nucleotide sequence that is at least about 60%, 65%, 70%, 75%, 80%, 85%, 90%, 95%, or 100% complementary to the RNA corresponding to the target gene, wherein the second nucleotide sequence: (i) is 15 to 30 nucleotides in length; and (ii) comprises 15 or more modified nucleotides independently selected from a 2′-O-methyl nucleotide and a 2′-fluoro nucleotide, wherein at least one modified nucleotide is a 2′-O-methyl nucleotide and at least one modified nucleotide is a 2′-fluoro nucleotide.

A double-stranded short interfering nucleic acid (ds-siNA) molecule of the disclosure may comprise: (a) a sense strand comprising a first nucleotide sequence that is at least about 60%, 65%, 70%, 75%, 80%, 85%, 90%, 95%, or 100% identical to an RNA corresponding to a target gene, wherein the first nucleotide sequence: (i) is 15 to 30 nucleotides in length; and (ii) comprises 15 or more modified nucleotides independently selected from a 2′-O-methyl nucleotide and a 2′-fluoro nucleotide, wherein at least one modified nucleotide is a 2′-O-methyl nucleotide and the nucleotide at position 7, 9, 10, and/or 11 from the 5′ end of the first nucleotide sequence is a 2′-fluoro nucleotide; and (b) an antisense strand comprising a second nucleotide sequence that is at least about 60%, 65%, 70%, 75%, 80%, 85%, 90%, 95%, or 100% complementary to the RNA corresponding to the target gene, wherein the second nucleotide sequence: (i) is 15 to 30 nucleotides in length; and (ii) comprises 15 or more modified nucleotides independently selected from a 2′-O-methyl nucleotide and a 2′-fluoro nucleotide, wherein at least one modified nucleotide is a 2′-O-methyl nucleotide and at least one modified nucleotide is a 2′-fluoro nucleotide.

A double-stranded short interfering nucleic acid (ds-siNA) molecule of the disclosure may comprise: (a) a sense strand comprising a first nucleotide sequence that is at least about 60%, 65%, 70%, 75%, 80%, 85%, 90%, 95%, or 100% identical to an RNA corresponding to a target gene, wherein the first nucleotide sequence: (i) is 15 to 30 nucleotides in length; and (ii) comprises 15 or more modified nucleotides independently selected from a 2′-O-methyl nucleotide and a 2′-fluoro nucleotide, wherein at least one modified nucleotide is a 2′-O-methyl nucleotide and at least one modified nucleotide is a 2′-fluoro nucleotide; and (b) an antisense strand comprising a second nucleotide sequence that is at least about 60%, 65%, 70%, 75%, 80%, 85%, 90%, 95%, or 100% complementary to the RNA corresponding to the target gene, wherein the second nucleotide sequence: (i) is 15 to 30 nucleotides in length; and (ii) comprises 15 or more modified nucleotides independently selected from a 2′-O-methyl nucleotide and a 2′-fluoro nucleotide, wherein at least one modified nucleotide is a 2′-O-methyl nucleotide and the nucleotide at position 2, 5, 6, 8, 10, 14, 16, 17, and/or 18 from the 5′ end of the second nucleotide sequence is a 2′-fluoro nucleotide.

A double-stranded short interfering nucleic acid (ds-siNA) molecule of the disclosure may comprise: (a) a sense strand comprising a first nucleotide sequence that is at least about 60%, 65%, 70%, 75%, 80%, 85%, 90%, 95%, or 100% identical to an RNA corresponding to a target gene, wherein the first nucleotide sequence: (i) is 15 to 30 nucleotides in length; and (ii) comprises 15 or more modified nucleotides independently selected from a 2′-O-methyl nucleotide and a 2′-fluoro nucleotide, wherein at least one modified nucleotide is a 2′-O-methyl nucleotide and at least one modified nucleotide is a 2′-fluoro nucleotide; and (b) an antisense strand comprising a second nucleotide sequence that is at least about 60%, 65%, 70%, 75%, 80%, 85%, 90%, 95%, or 100% complementary to the RNA corresponding to the target gene, wherein the second nucleotide sequence: (i) is 15 to 30 nucleotides in length; and (ii) comprises 15 or more modified nucleotides independently selected from a 2′-O-methyl nucleotide and a 2′-fluoro nucleotide, wherein at least one modified nucleotide is a 2′-O-methyl nucleotide and the nucleotide at position 2 of the second nucleotide sequence is a 2′-fluoro nucleotide.

A double-stranded short interfering nucleic acid (ds-siNA) molecule of the disclosure may comprise: (a) a sense strand comprising a first nucleotide sequence that is at least about 60%, 65%, 70%, 75%, 80%, 85%, 90%, 95%, or 100% identical to an RNA corresponding to a target gene, wherein the first nucleotide sequence: (i) is 15 to 30 nucleotides in length; (ii) comprises 15 or more modified nucleotides independently selected from a 2′-O-methyl nucleotide and a 2′-fluoro nucleotide, wherein at least one modified nucleotide is a 2′-O-methyl nucleotide and at least one modified nucleotide is a 2′-fluoro nucleotide; and (iii) comprises 1 or more phosphorothioate internucleoside linkage; and (b) an antisense strand comprising a second nucleotide sequence that is at least about 60%, 65%, 70%, 75%, 80%, 85%, 90%, 95%, or 100% complementary to the RNA corresponding to the target gene, wherein the second nucleotide sequence: (i) is 15 to 30 nucleotides in length; (ii) comprises 15 or more modified nucleotides independently selected from a 2′-O-methyl nucleotide and a 2′-fluoro nucleotide, wherein at least one modified nucleotide is a 2′-O-methyl nucleotide and at least one modified nucleotide is a 2′-fluoro nucleotide; and (iii) comprises 1 or more phosphorothioate internucleoside linkage.

A double-stranded short interfering nucleic acid (ds-siNA) molecule of the disclosure may comprise: (a) a sense strand comprising a first nucleotide sequence that is at least about 60%, 65%, 70%, 75%, 80%, 85%, 90%, 95%, or 100% identical to an RNA corresponding to a target gene, wherein the first nucleotide sequence: (i) is 15 to 30 nucleotides in length; and (ii) comprises 15 or more modified nucleotides independently selected from a 2′-O-methyl nucleotide and a 2′-fluoro nucleotide, wherein at least one modified nucleotide is a 2′-O-methyl nucleotide and at least one modified nucleotide is a 2′-fluoro nucleotide; and (b) an antisense strand comprising a second nucleotide sequence that is at least about 60%, 65%, 70%, 75%, 80%, 85%, 90%, 95%, or 100% complementary to the RNA corresponding to the target gene, wherein the second nucleotide sequence: (i) is 15 to 30 nucleotides in length; and (ii) comprises 15 or more modified nucleotides independently selected from a 2′-O-methyl nucleotide and a 2′-fluoro nucleotide, wherein at least one modified nucleotide is a 2′-O-methyl nucleotide and at least one modified nucleotide is a 2′-fluoro nucleotide, wherein the ds-siNA may further comprise a phosphorylation blocker and/or a 5′-stabilized end cap.

A double-stranded short interfering nucleic acid (ds-siNA) molecule comprises: (I) a sense strand comprising (A) a first nucleotide sequence that is at least about 60%, 65%, 70%, 75%, 80%, 85%, 90%, 95%, or 100% identical to an RNA corresponding to a target gene, wherein the first nucleotide sequence: (i) is 15 to 30 nucleotides in length; and (ii) comprises 15 or more modified nucleotides independently selected from a 2′-O-methyl nucleotide and a 2′-fluoro nucleotide, wherein at least one modified nucleotide is a 2′-O-methyl nucleotide and at least one modified nucleotide is a 2′-fluoro nucleotide; and (B) a phosphorylation blocker; and (II) an antisense strand comprising a second nucleotide sequence that is at least about 60%, 65%, 70%, 75%, 80%, 85%, 90%, 95%, or 100% complementary to the RNA corresponding to the target gene, wherein the second nucleotide sequence: (a) is 15 to 30 nucleotides in length; and (b) comprises 15 or more modified nucleotides independently selected from a 2′-O-methyl nucleotide and a 2′-fluoro nucleotide, wherein at least one modified nucleotide is a 2′-O-methyl nucleotide and at least one modified nucleotide is a 2′-fluoro nucleotide.

A double-stranded short interfering nucleic acid (ds-siNA) molecule of the disclosure may comprise: (I) a sense strand comprising a first nucleotide sequence that is at least about 60%, 65%, 70%, 75%, 80%, 85%, 90%, 95%, or 100% identical to an RNA corresponding to a target gene, wherein the first nucleotide sequence: (a) is 15 to 30 nucleotides in length; and (b) comprises 15 or more modified nucleotides independently selected from a 2′-O-methyl nucleotide and a 2′-fluoro nucleotide, wherein at least one modified nucleotide is a 2′-O-methyl nucleotide and at least one modified nucleotide is a 2′-fluoro nucleotide; and (II) an antisense strand comprising (A) a second nucleotide sequence that is at least about 60%, 65%, 70%, 75%, 80%, 85%, 90%, 95%, or 100% complementary to the RNA corresponding to the target gene, wherein the second nucleotide sequence: (i) is 15 to 30 nucleotides in length; and (ii) comprises 15 or more modified nucleotides independently selected from a 2′-O-methyl nucleotide and a 2′-fluoro nucleotide, wherein at least one modified nucleotide is a 2′-O-methyl nucleotide and at least one modified nucleotide is a 2′-fluoro nucleotide; and (B) a 5′-stabilized end cap.

A double-stranded short interfering nucleic acid (ds-siNA) molecule of the disclosure may comprise: (I) a sense strand comprising (A) a first nucleotide sequence that is at least about 60%, 65%, 70%, 75%, 80%, 85%, 90%, 95%, or 100% identical to an RNA corresponding to a target gene, wherein the first nucleotide sequence: (i) is 15 to 30 nucleotides in length; and (ii) comprises 15 or more modified nucleotides independently selected from a 2′-O-methyl nucleotide and a 2′-fluoro nucleotide, wherein at least one modified nucleotide is a 2′-O-methyl nucleotide and at least one modified nucleotide is a 2′-fluoro nucleotide; and (B) a phosphorylation blocker; and (II) an antisense strand comprising (A) a second nucleotide sequence that is at least about 60%, 65%, 70%, 75%, 80%, 85%, 90%, 95%, or 100% complementary to the RNA corresponding to the target gene, wherein the second nucleotide sequence: (i) is 15 to 30 nucleotides in length; and (ii) comprises 15 or more modified nucleotides independently selected from a 2′-O-methyl nucleotide and a 2′-fluoro nucleotide, wherein at least one modified nucleotide is a 2′-O-methyl nucleotide and at least one modified nucleotide is a 2′-fluoro nucleotide; and (B) a 5′-stabilized end cap.

A double-stranded short interfering nucleic acid (ds-siNA) molecule of the disclosure may comprise: (a) a sense strand comprising a first nucleotide sequence that is at least about 60%, 65%, 70%, 75%, 80%, 85%, 90%, 95%, or 100% identical to an RNA corresponding to a target gene, wherein the first nucleotide sequence comprises a nucleotide sequence of any one of the sequences disclosed in Table 1; and (b) an antisense strand comprising a second nucleotide sequence that is at least about 60%, 65%, 70%, 75%, 80%, 85%, 90%, 95%, or 100% complementary to the RNA corresponding to the target gene, wherein the second nucleotide sequence comprises a nucleotide sequence of any one of the sequences disclosed in Table 1.

A double-stranded short interfering nucleic acid (ds-siNA) molecule comprises: (a) a sense strand comprising a first nucleotide sequence that is at least about 60%, 65%, 70%, 75%, 80%, 85%, 90%, 95%, or 100% identical to an RNA corresponding to a target gene, wherein the first nucleotide sequence comprises a nucleotide sequence as shown in Table 2; and (b) an antisense strand comprising a second nucleotide sequence that is at least about 60%, 65%, 70%, 75%, 80%, 85%, 90%, 95%, or 100% complementary to the RNA corresponding to the target gene, wherein the second nucleotide sequence comprises a nucleotide sequence as shown in Table 2.

Further disclosed herein are compositions comprising two or more of the siNA molecules described herein. Further disclosed herein are compositions comprising any of the siNA molecule described and a pharmaceutically acceptable carrier or diluent. Further disclosed herein are compositions comprising two or more of the siNA molecules described herein for use as a medicament. Further disclosed herein are compositions comprising any of the siNA molecule described and a pharmaceutically acceptable carrier or diluent for use as a medicament.

Further disclosed herein are methods of treating an infection (e.g., COVID-19) in a subject in need thereof, the method comprising administering to the subject any of the siNA molecules described herein. Further disclosed herein are uses of any of the siNA molecules described herein in the manufacture of a medicament for treating an infection (e.g., COVID-19).

A. siNA Sense Strand

Any of the siNA molecules or oligomers described herein may comprise a sense strand. The sense strand may comprise a first nucleotide sequence. The first nucleotide sequence may be 15 to 30, 15 to 25, 15 to 23, 17 to 23, 19 to 23, or 19 to 21 nucleotides in length. In some embodiments, the first nucleotide sequence is 15, 16, 17, 18, 19, 20, 21, 22, 23, 24, 25, 26, 27, 28, 29, or 30 nucleotides in length. In some embodiments, the first nucleotide sequence is at least 19 nucleotides in length. In some embodiments, the first nucleotide sequence is at least 21 nucleotides in length.

In some embodiments, the sense strand is the same length as the first nucleotide sequence. In some embodiments, the sense strand is longer than the first nucleotide sequence. In some embodiments, the sense strand may further comprise 1, 2, 3, 4, or 5 or more nucleotides than the first nucleotide sequence. In some embodiments, the sense strand may further comprise a deoxyribonucleic acid (DNA). In some embodiments, the DNA is thymine (T). In some embodiments, the sense strand may further comprise a TT sequence. In some embodiments, the TT sequence is adjacent to the first nucleotide sequence. In some embodiments, the sense strand may further comprise one or more modified nucleotides that are adjacent to the first nucleotide sequence. In some embodiments, the one or more modified nucleotides are independently selected from any of the modified nucleotides disclosed herein (e.g., 2′-fluoro nucleotide, 2′-O-methyl nucleotide, 2′-fluoro nucleotide mimic, 2′-O-methyl nucleotide mimic, or a nucleotide comprising a modified nucleobase).

In some embodiments, at least one end of the ds-siNA may be a blunt end. In some embodiments, at least one end of the ds-siNA may comprise an overhang, wherein the overhang comprises at least one nucleotide. In some embodiments, both ends of the ds-siNA may comprise an overhang, wherein the overhang comprises at least one nucleotide.

In some embodiments, the first nucleotide sequence comprises 15, 16, 17, 18, 19, 20, 21, 22, 23, or more modified nucleotides independently selected from a 2′-O-methyl nucleotide and a 2′-fluoro nucleotide. In some embodiments, 70%, 75%, 80%, 85%, 90%, 95% or 100% of the nucleotides in the first nucleotide sequence are modified nucleotides independently selected from a 2′-O-methyl nucleotide and a 2′-fluoro nucleotide. In some embodiments, 100% of the nucleotides in the first nucleotide sequence are modified nucleotides independently selected from a 2′-O-methyl nucleotide and a 2′-fluoro nucleotide. In some embodiments, the 2′-O-methyl nucleotide is a 2′-O-methyl nucleotide mimic. In some embodiments, the 2′-fluoro nucleotide is a 2′-fluoro nucleotide mimic.

In some embodiments, between about 15 to 30, 15 to 25, 15 to 24, 15 to 23, 15 to 22, 15 to 21, 17 to 30, 17 to 25, 17 to 24, 17 to 23, 17 to 22, 17 to 21, 18 to 30, 18 to 25, 18 to 24, 18 to 23, 18 to 22, 18 to 21, 19 to 30, 19 to 25, 19 to 24, 19 to 23, 19 to 22, 19 to 21, 20 to 25, 20 to 24, 20 to 23, 21 to 25, 21 to 24, or 21 to 23 modified nucleotides of the first nucleotide sequence are 2′-O-methyl nucleotides. In some embodiments, between about 2 to 20 modified nucleotides of the first nucleotide sequence are 2′-O-methyl nucleotides. In some embodiments, between about 5 to 25 modified nucleotides of the first nucleotide sequence are 2′-O-methyl nucleotides. In some embodiments, between about 10 to 25 modified nucleotides of the first nucleotide sequence are 2′-O-methyl nucleotides. In some embodiments, between about 12 to 25 modified nucleotides of the first nucleotide sequence are 2′-O-methyl nucleotides. In some embodiments, at least about 10, 11, 12, 13, 14, 15, 16, 17, 18, 19, 20, 21, or 22 modified nucleotides of the first nucleotide sequence are 2′-O-methyl nucleotides. In some embodiments, at least about 12 modified nucleotides of the first nucleotide sequence are 2′-O-methyl nucleotides. In some embodiments, at least about 13 modified nucleotides of the first nucleotide sequence are 2′-O-methyl nucleotides. In some embodiments, at least about 14 modified nucleotides of the first nucleotide sequence are 2′-O-methyl nucleotides. In some embodiments, at least about 15 modified nucleotides of the first nucleotide sequence are 2′-O-methyl nucleotides. In some embodiments, at least about 16 modified nucleotides of the first nucleotide sequence are 2′-O-methyl nucleotides. In some embodiments, at least about 17 modified nucleotides of the first nucleotide sequence are 2′-O-methyl nucleotides. In some embodiments, at least about 18 modified nucleotides of the first nucleotide sequence are 2′-O-methyl nucleotides. In some embodiments, at least about 19 modified nucleotides of the first nucleotide sequence are 2′-O-methyl nucleotides. In some embodiments, less than or equal to 25, 24, 23, 22, 21, 20, 19, 18, 17, 16, 15, 14, 13, 12, 11, 10, 9, 8, 7, 6, 5, 4, 3, or 2 modified nucleotides of the first nucleotide sequence are 2′-O-methyl nucleotides. In some embodiments, less than or equal to 21 modified nucleotides of the first nucleotide sequence are 2′-O-methyl nucleotides. In some embodiments, less than or equal to 20 modified nucleotides of the first nucleotide sequence are 2′-O-methyl nucleotides. In some embodiments, less than or equal to 19 modified nucleotides of the first nucleotide sequence are 2′-O-methyl nucleotides. In some embodiments, less than or equal to 18 modified nucleotides of the first nucleotide sequence are 2′-O-methyl nucleotides. In some embodiments, less than or equal to 17 modified nucleotides of the first nucleotide sequence are 2′-O-methyl nucleotides. In some embodiments, less than or equal to 16 modified nucleotides of the first nucleotide sequence are 2′-O-methyl nucleotides. In some embodiments, less than or equal to 15 modified nucleotides of the first nucleotide sequence are 2′-O-methyl nucleotides. In some embodiments, less than or equal to 14 modified nucleotides of the first nucleotide sequence are 2′-O-methyl nucleotides. In some embodiments, less than or equal to 13 modified nucleotides of the first nucleotide sequence are 2′-O-methyl nucleotides. In some embodiments, at least one modified nucleotide of the first nucleotide sequence is a 2′-O-methyl pyrimidine. In some embodiments, at least 5, 6, 7, 8, 9, or 10 modified nucleotides of the first nucleotide sequence are 2′-O-methyl pyrimidines. In some embodiments, at least one modified nucleotide of the first nucleotide sequence is a 2′-O-methyl purine. In some embodiments, at least 5, 6, 7, 8, 9, or 10 modified nucleotides of the first nucleotide sequence are 2′-O-methyl purines. In some embodiments, the 2′-O-methyl nucleotide is a 2′-O-methyl nucleotide mimic.

In some embodiments, between 2 to 15 modified nucleotides of the first nucleotide sequence are 2′-fluoro nucleotides. In some embodiments, between 2 to 10 modified nucleotides of the first nucleotide sequence are 2′-fluoro nucleotides. In some embodiments, between 2 to 6 modified nucleotides of the first nucleotide sequence are 2′-fluoro nucleotides. In some embodiments, 1 to 6, 1 to 5, 1 to 4, or 1 to 3 modified nucleotides of the first nucleotide sequence are 2′-fluoro nucleotides. In some embodiments, at least 1, 2, 3, 4, 5, or 6 modified nucleotides of the first nucleotide sequence are 2′-fluoro nucleotides. In some embodiments, at least 1 modified nucleotide of the first nucleotide sequence is a 2′-fluoro nucleotide. In some embodiments, at least 2 modified nucleotides of the first nucleotide sequence are 2′-fluoro nucleotides. In some embodiments, at least 3 modified nucleotides of the first nucleotide sequence are 2′-fluoro nucleotides. In some embodiments, at least 4 modified nucleotides of the first nucleotide sequence are 2′-fluoro nucleotides. In some embodiments, at least 5 modified nucleotides of the first nucleotide sequence are 2′-fluoro nucleotides. In some embodiments, at least 6 modified nucleotides of the first nucleotide sequence are 2′-fluoro nucleotides. In some embodiments, 10, 9, 8, 7, 6, 5, 4, 3 or fewer modified nucleotides of the first nucleotide sequence are 2′-fluoro nucleotides. In some embodiments, 10 or fewer modified nucleotides of the first nucleotide sequence are 2′-fluoro nucleotides. In some embodiments, 7 or fewer modified nucleotides of the first nucleotide sequence are 2′-fluoro nucleotides. In some embodiments, 6 or fewer modified nucleotides of the first nucleotide sequence are 2′-fluoro nucleotides. In some embodiments, 5 or fewer modified nucleotides of the first nucleotide sequence are 2′-fluoro nucleotides. In some embodiments, 4 or fewer modified nucleotides of the first nucleotide sequence are 2′-fluoro nucleotides. In some embodiments, 3 or fewer modified nucleotides of the first nucleotide sequence are 2′-fluoro nucleotides. In some embodiments, 2 or fewer modified nucleotides of the first nucleotide sequence are 2′-fluoro nucleotides. In some embodiments, at least one modified nucleotide of the first nucleotide sequence is a 2′-fluoro pyrimidine. In some embodiments, 1, 2, 3, 4, 5, or 6 modified nucleotides of the first nucleotide sequence are 2′-fluoro pyrimidines. In some embodiments, at least one modified nucleotide of the first nucleotide sequence is a 2′-fluoro purine. In some embodiments, 1, 2, 3, 4, 5, or 6 modified nucleotides of the first nucleotide sequence are 2′-fluoro purines. In some embodiments, the 2′-fluoro nucleotide is a 2′-fluoro nucleotide mimic.

In some embodiments, the nucleotide at position 3, 5, 7, 8, 9, 10, 11, 12, 14, 17, and/or 19 from the 5′ end of the first nucleotide sequence is a 2′-fluoro nucleotide. In some embodiments, at least two nucleotides at positions 3, 5, 7, 8, 9, 10, 11, 12, 14, 17, and/or 19 from the 5′ end of the first nucleotide sequence are 2′-fluoro nucleotides. In some embodiments, at least three nucleotides at positions 3, 5, 7, 8, 9, 10, 11, 12, 14, 17, and/or 19 from the 5′ end of the first nucleotide sequence are 2′-fluoro nucleotides. In some embodiments, at least four nucleotides at positions 3, 5, 7, 8, 9, 10, 11, 12, 14, 17, and/or 19 from the 5′ end of the first nucleotide sequence are 2′-fluoro nucleotides. In some embodiments, at least five nucleotides at positions 3, 5, 7, 8, 9, 10, 11, 12, 14, 17, and/or 19 from the 5′ end of the first nucleotide sequence are 2′-fluoro nucleotides. In some embodiments, the nucleotides at positions 3, 5, 7, 8, 9, 10, 11, 12, 14, 17, and/or 19 from the 5′ end of the first nucleotide sequence are 2′-fluoro nucleotides. In some embodiments, the nucleotide at position 3 from the 5′ end of the first nucleotide sequence is a 2′-fluoro nucleotide. In some embodiments, the nucleotide at position 7 from the 5′ end of the first nucleotide sequence is a 2′-fluoro nucleotide. In some embodiments, the nucleotide at position 8 from the 5′ end of the first nucleotide sequence is a 2′-fluoro nucleotide. In some embodiments, the nucleotide at position 9 from the 5′ end of the first nucleotide sequence is a 2′-fluoro nucleotide. In some embodiments, the nucleotide at position 12 from the 5′ end of the first nucleotide sequence is a 2′-fluoro nucleotide. In some embodiments, the nucleotide at position 17 from the 5′ end of the first nucleotide sequence is a 2′-fluoro nucleotide. In some embodiments, the 2′-fluoro nucleotide is a 2′-fluoro nucleotide mimic.

In some embodiments, at least 1, 2, 3, 4, 5, 6, or 7 nucleotides at position 3, 5, 7, 8, 9, 10, 11, 12, 14, 17, and/or 19 from the 5′ end of the first nucleotide sequence is a 2′-fluoro nucleotide. In some embodiments, the nucleotide at positions 3, 5, 7, 8, 9, 10, 11, 12, 14, 17, and/or 19 from the 5′ end of the first nucleotide sequence is a 2′-fluoro nucleotide. In some embodiments, at least two nucleotides at positions 3, 5, 7, 8, 9, 10, 11, 12, 14, 17, and/or 19 from the 5′ end of the first nucleotide sequence are 2′-fluoro nucleotides. In some embodiments, at least three nucleotides at positions 3, 5, 7, 8, 9, 10, 11, 12, 14, 17, and/or 19 from the 5′ end of the first nucleotide sequence are 2′-fluoro nucleotides. In some embodiments, the nucleotides at positions 3, 5, 7, 8, 9, 10, 11, 12, 14, 17, and/or 19 from the 5′ end of the first nucleotide sequence are 2′-fluoro nucleotides. In some embodiments, the nucleotide at position 3 from the 5′ end of the first nucleotide sequence is a 2′-fluoro nucleotide. In some embodiments, the nucleotide at position 5 from the 5′ end of the first nucleotide sequence is a 2′-fluoro nucleotide. In some embodiments, the nucleotide at position 7 from the 5′ end of the first nucleotide sequence is a 2′-fluoro nucleotide. In some embodiments, the nucleotide at position 8 from the 5′ end of the first nucleotide sequence is a 2′-fluoro nucleotide. In some embodiments, the nucleotide at position 9 from the 5′ end of the first nucleotide sequence is a 2′-fluoro nucleotide. In some embodiments, the nucleotide at position 10 from the 5′ end of the first nucleotide sequence is a 2′-fluoro nucleotide. In some embodiments, the nucleotide at position 11 from the 5′ end of the first nucleotide sequence is a 2′-fluoro nucleotide. In some embodiments, the nucleotide at position 12 from the 5′ end of the first nucleotide sequence is a 2′-fluoro nucleotide. In some embodiments, the nucleotide at position 14 from the 5′ end of the first nucleotide sequence is a 2′-fluoro nucleotide. In some embodiments, the nucleotide at position 17 from the 5′ end of the first nucleotide sequence is a 2′-fluoro nucleotide. In some embodiments, the nucleotide at position 19 from the 5′ end of the first nucleotide sequence is a 2′-fluoro nucleotide. In some embodiments, the nucleotide at position 3, 7, 8, 9, 12, and/or 17 from the 5′ end of the first nucleotide sequence is a 2′-fluoro nucleotide. In some embodiments, the nucleotide at position 3, 7, 8, and/or 17 from the 5′ end of the first nucleotide sequence is a 2′-fluoro nucleotide. In some embodiments, the nucleotide at position 3, 7, 8, 9, 12, and/or 17 from the 5′ end of the first nucleotide sequence is a 2′-fluoro nucleotide. In some embodiments, the nucleotide at position 5, 7, 8, and/or 9 from the 5′ end of the first nucleotide sequence is a 2′-fluoro nucleotide. In some embodiments, the nucleotide at position 5, 9, 10, 11, 12, and/or 19 from the 5′ end of the first nucleotide sequence is a 2′-fluoro nucleotide. In some embodiments, the 2′-fluoro nucleotide is a 2′-fluoro nucleotide mimic. The 2′-fluoro nucleotide mimic can be selected from

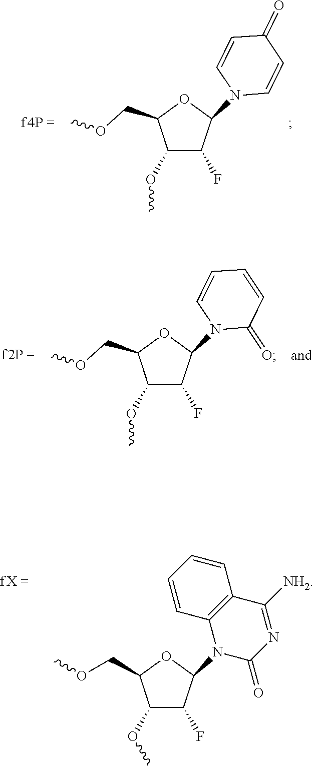

In some embodiments, the 2′-fluoro nucleotide or 2′-O-methyl nucleotide is a 2′-fluoro or 2′-O-methyl nucleotide mimic. In some embodiments, the 2′-fluoro or 2′-O-methyl nucleotide mimic is a nucleotide mimic of Formula (V):

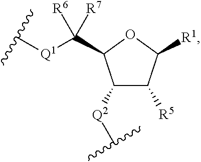

wherein R¹ is a independently nucleobase, aryl, heteroaryl, or H, Q¹ and Q² are independently S or O, R⁵ is independently —OCD₃, —F, or —OCH₃, and R⁶ and R⁷ are independently H, D, or CD₃. In some embodiments, the nucleobase is selected from cytosine, guanine, adenine, uracil, aryl, heteroaryl, and an analogue or derivative thereof.

In some embodiments, the 2′-fluoro or 2′-O-methyl nucleotide mimic is a nucleotide mimic of Formula (16)-Formula (20):

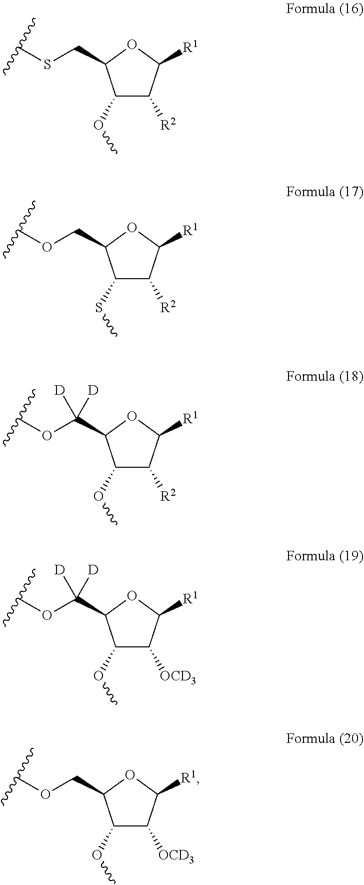

wherein R¹ is independently a nucleobase and R² is F or —OCH₃. In some embodiments, the nucleobase is selected from cytosine, guanine, adenine, uracil, aryl, heteroaryl, and an analogue or derivative thereof.

In some embodiments, the first nucleotide sequence comprises, consists of, or consists essentially of ribonucleic acids (RNAs). In some embodiments, the first nucleotide sequence comprises, consists of, or consists essentially of modified RNAs. In some embodiments, the modified RNAs are selected from a 2′-O-methyl RNA and 2′-fluoro RNA. In some embodiments, 15, 16, 17, 18, 19, 20, 21, 22, or 23 modified nucleotides of the first nucleotide sequence are independently selected from 2′-O-methyl RNA and 2′-fluoro RNA.

In some embodiments, the sense strand may further comprise one or more internucleoside linkages independently selected from a phosphodiester (PO) internucleoside linkage, phosphorothioate (PS) internucleoside linkage, phosphorodithioate internucleoside linkage, and PS-mimic internucleoside linkage. In some embodiments, the PS-mimic internucleoside linkage is a sulfo internucleotide linkage.

In some embodiments, the sense strand may further comprise at least 1, 2, 3, 4, 5, 6, 7, 8, 9, 10, 11, 12, 13, 14, or 15 or more phosphorothioate internucleoside linkages. In some embodiments, the sense strand comprises 20, 19, 18, 17, 16, 15, 14, 13, 12, 11, 10, 9, 8, 7, 6, 5, 4, or 3 or fewer phosphorothioate internucleoside linkages. In some embodiments, the sense strand comprises 2 to 10, 2 to 8, 2 to 6, 1 to 5, 1 to 4, 1 to 3, or 1 to 2 phosphorothioate internucleoside linkages. In some embodiments, the sense strand comprises 1 to 2 phosphorothioate internucleoside linkages. In some embodiments, the sense strand comprises 2 to 4 phosphorothioate internucleoside linkages. In some embodiments, at least one phosphorothioate internucleoside linkage is between the nucleotides at positions 1 and 2 from the 5′ end of the first nucleotide sequence. In some embodiments, at least one phosphorothioate internucleoside linkage is between the nucleotides at positions 2 and 3 from the 5′ end of the first nucleotide sequence. In some embodiments, the sense strand comprises two phosphorothioate internucleoside linkages between the nucleotides at positions 1 to 3 from the 5′ end of the first nucleotide sequence.

In some embodiments, any of the sense strands disclosed herein may comprise a 5′ end cap monomer. In some embodiments, any of the first nucleotide sequences disclosed herein may comprise a 5′ end cap monomer.

B. siNA Antisense Strand

Any of the siNA molecules described herein may comprise an antisense strand. The antisense strand may comprise a second nucleotide sequence. The second nucleotide sequence may be 15 to 30, 15 to 25, 15 to 23, 17 to 23, 19 to 23, or 19 to 21 nucleotides in length. In some embodiments, the second nucleotide sequence is 15, 16, 17, 18, 19, 20, 21, 22, 23, 24, 25, 26, 27, 28, 29, or 30 nucleotides in length. In some embodiments, the second nucleotide sequence is at least 19 nucleotides in length. In some embodiments, the second nucleotide sequence is at least 21 nucleotides in length.

In some embodiments, the antisense strand is the same length as the second nucleotide sequence. In some embodiments, the antisense strand is longer than the second nucleotide sequence. In some embodiments, the antisense strand may further comprise 1, 2, 3, 4, or 5 or more nucleotides than the second nucleotide sequence. In some embodiments, the antisense strand is the same length as the sense strand. In some embodiments, the antisense strand is longer than the sense strand. In some embodiments, the antisense strand may further comprise 1, 2, 3, 4, or 5 or more nucleotides than the sense strand. In some embodiments, the antisense strand may further comprise a deoxyribonucleic acid (DNA). In some embodiments, the DNA is thymine (T). In some embodiments, the antisense strand may further comprise a TT sequence. In some embodiments, the antisense strand may further comprise one or more modified nucleotides that are adjacent to the second nucleotide sequence. In some embodiments, the one or more modified nucleotides are independently selected from any of the modified nucleotides disclosed herein (e.g., 2′-fluoro nucleotide, 2′-O-methyl nucleotide, 2′-fluoro nucleotide mimic, 2′-O-methyl nucleotide mimic, or a nucleotide comprising a modified nucleobase).

In some embodiments, the second nucleotide sequence comprises 15, 16, 17, 18, 19, 20, 21, 22, 23, or more modified nucleotides independently selected from a 2′-O-methyl nucleotide and a 2′-fluoro nucleotide. In some embodiments, 70%, 75%, 80%, 85%, 90%, 95% or 100% of the nucleotides in the second nucleotide sequence are modified nucleotides independently selected from a 2′-O-methyl nucleotide and a 2′-fluoro nucleotide. In some embodiments, 100% of the nucleotides in the second nucleotide sequence are modified nucleotides independently selected from a 2′-O-methyl nucleotide and a 2′-fluoro nucleotide.

In some embodiments, between about 15 to 30, 15 to 25, 15 to 24, 15 to 23, 15 to 22, 15 to 21, 17 to 30, 17 to 25, 17 to 24, 17 to 23, 17 to 22, 17 to 21, 18 to 30, 18 to 25, 18 to 24, 18 to 23, 18 to 22, 18 to 21, 19 to 30, 19 to 25, 19 to 24, 19 to 23, 19 to 22, 19 to 21, 20 to 25, 20 to 24, 20 to 23, 21 to 25, 21 to 24, or 21 to 23 modified nucleotides of the second nucleotide sequence are 2′-O-methyl nucleotides. In some embodiments, between about 2 to 20 modified nucleotides of the second nucleotide sequence are 2′-O-methyl nucleotides. In some embodiments, between about 5 to 25 modified nucleotides of the second nucleotide sequence are 2′-O-methyl nucleotides. In some embodiments, between about 10 to 25 modified nucleotides of the second nucleotide sequence are 2′-O-methyl nucleotides. In some embodiments, between about 12 to 25 modified nucleotides of the second nucleotide sequence are 2′-O-methyl nucleotides. In some embodiments, at least 10, 11, 12, 13, 14, 15, 16, 17, 18, 19, 20, 21, or 22 modified nucleotides of the second nucleotide sequence are 2′-O-methyl nucleotides. In some embodiments, at least about 12 modified nucleotides of the second nucleotide sequence are 2′-O-methyl nucleotides. In some embodiments, at least about 13 modified nucleotides of the second nucleotide sequence are 2′-O-methyl nucleotides. In some embodiments, at least about 14 modified nucleotides of the second nucleotide sequence are 2′-O-methyl nucleotides. In some embodiments, at least about 15 modified nucleotides of the second nucleotide sequence are 2′-O-methyl nucleotides. In some embodiments, at least about 16 modified nucleotides of the second nucleotide sequence are 2′-O-methyl nucleotides. In some embodiments, at least about 17 modified nucleotides of the second nucleotide sequence are 2′-O-methyl nucleotides. In some embodiments, at least about 18 modified nucleotides of the second nucleotide sequence are 2′-O-methyl nucleotides. In some embodiments, at least about 19 modified nucleotides of the second nucleotide sequence are 2′-O-methyl nucleotides. In some embodiments, less than or equal to 25, 24, 23, 22, 21, 20, 19, 18, 17, 16, 15, 14, 13, 12, 11, 10, 9, 8, 7, 6, 5, 4, 3, or 2 modified nucleotides of the second nucleotide sequence are 2′-O-methyl nucleotides. In some embodiments, less than or equal to 21 modified nucleotides of the second nucleotide sequence are 2′-O-methyl nucleotides. In some embodiments, less than or equal to 20 modified nucleotides of the second nucleotide sequence are 2′-O-methyl nucleotides. In some embodiments, less than or equal to 19 modified nucleotides of the second nucleotide sequence are 2′-O-methyl nucleotides. In some embodiments, less than or equal to 18 modified nucleotides of the second nucleotide sequence are 2′-O-methyl nucleotides. In some embodiments, less than or equal to 17 modified nucleotides of the second nucleotide sequence are 2′-O-methyl nucleotides. In some embodiments, less than or equal to 16 modified nucleotides of the second nucleotide sequence are 2′-O-methyl nucleotides. In some embodiments, less than or equal to 15 modified nucleotides of the second nucleotide sequence are 2′-O-methyl nucleotides. In some embodiments, less than or equal to 14 modified nucleotides of the second nucleotide sequence are 2′-O-methyl nucleotides. In some embodiments, less than or equal to 13 modified nucleotides of the second nucleotide sequence are 2′-O-methyl nucleotides. In some embodiments, at least one modified nucleotide of the second nucleotide sequence is a 2′-O-methyl pyrimidine. In some embodiments, at least 5, 6, 7, 8, 9, or 10 modified nucleotides of the second nucleotide sequence are 2′-O-methyl pyrimidines. In some embodiments, at least one modified nucleotide of the second nucleotide sequence is a 2′-O-methyl purine. In some embodiments, at least 5, 6, 7, 8, 9, or 10 modified nucleotides of the second nucleotide sequence are 2′-O-methyl purines. In some embodiments, the 2′-O-methyl nucleotide is a 2′-O-methyl nucleotide mimic.

In some embodiments, between 2 to 15 modified nucleotides of the second nucleotide sequence are 2′-fluoro nucleotides. In some embodiments, between 2 to 10 modified nucleotides of the second nucleotide sequence are 2′-fluoro nucleotides. In some embodiments, between 2 to 6 modified nucleotides of the second nucleotide sequence are 2′-fluoro nucleotides. In some embodiments, 1 to 6, 1 to 5, 1 to 4, or 1 to 3 modified nucleotides of the second nucleotide sequence are 2′-fluoro nucleotides. In some embodiments, at least 1, 2, 3, 4, 5, or 6 modified nucleotides of the second nucleotide sequence are 2′-fluoro nucleotides. In some embodiments, at least 1 modified nucleotide of the second nucleotide sequence is a 2′-fluoro nucleotide. In some embodiments, at least 2 modified nucleotides of the second nucleotide sequence are 2′-fluoro nucleotides. In some embodiments, at least 3 modified nucleotides of the second nucleotide sequence are 2′-fluoro nucleotides. In some embodiments, at least 4 modified nucleotides of the second nucleotide sequence are 2′-fluoro nucleotides. In some embodiments, at least 5 modified nucleotides of the second nucleotide sequence are 2′-fluoro nucleotides. In some embodiments, 10, 9, 8, 7, 6, 5, 4, 3 or fewer modified nucleotides of the second nucleotide sequence are 2′-fluoro nucleotides. In some embodiments, 10 or fewer modified nucleotides of the second nucleotide sequence are 2′-fluoro nucleotides. In some embodiments, 7 or fewer modified nucleotides of the second nucleotide sequence are 2′-fluoro nucleotides. In some embodiments, 6 or fewer modified nucleotides of the second nucleotide sequence are 2′-fluoro nucleotides. In some embodiments, 5 or fewer modified nucleotides of the second nucleotide sequence are 2′-fluoro nucleotides. In some embodiments, 4 or fewer modified nucleotides of the second nucleotide sequence are 2′-fluoro nucleotides. In some embodiments, 3 or fewer modified nucleotides of the second nucleotide sequence are 2′-fluoro nucleotides. In some embodiments, 2 or fewer modified nucleotides of the second nucleotide sequence are 2′-fluoro nucleotides. In some embodiments, at least one modified nucleotide of the second nucleotide sequence is a 2′-fluoro pyrimidine. In some embodiments, 1, 2, 3, 4, 5, or 6 modified nucleotides of the second nucleotide sequence are 2′-fluoro pyrimidines. In some embodiments, at least one modified nucleotide of the second nucleotide sequence is a 2′-fluoro purine. In some embodiments, 1, 2, 3, 4, 5, or 6 modified nucleotides of the second nucleotide sequence are 2′-fluoro purines. In some embodiments, the 2′-fluoro nucleotide is a 2′-fluoro nucleotide mimic. The 2′-fluoro nucleotide mimic can be selected from

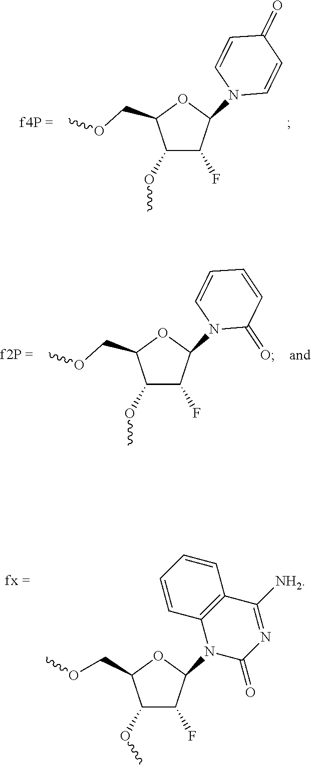

In some embodiments, the 2′-fluoro nucleotide or 2′-O-methyl nucleotide is a 2′-fluoro or 2′-O-methyl nucleotide mimic. In some embodiments, the 2′-fluoro or 2′-O-methyl nucleotide mimic is a nucleotide mimic of Formula (V):

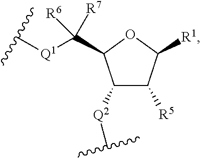

wherein R¹ is independently a nucleobase, aryl, heteroaryl, or H, Q¹ and Q² are independently S or O, R⁵ is independently —OCD₃, —F, or —OCH₃, and R⁶ and R⁷ are independently H, D, or CD₃. In some embodiments, the nucleobase is selected from cytosine, guanine, adenine, uracil, aryl, heteroaryl, and an analogue or derivative thereof.

In some embodiments, the 2′-fluoro or 2′-O-methyl nucleotide mimic is a nucleotide mimic of Formula (16)-Formula (20):

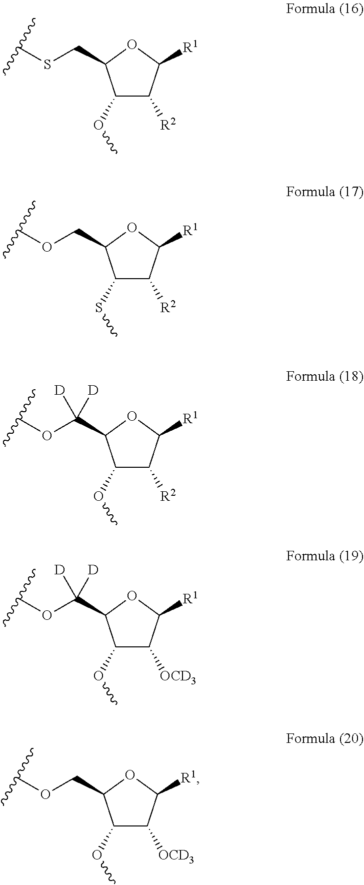

wherein R¹ is a nucleobase and R² is independently F or —OCH₃. In some embodiments, the nucleobase is selected from cytosine, guanine, adenine, uracil, aryl, heteroaryl, and an analogue or derivative thereof.

In some embodiments, at least 1, 2, 3, 4, 5, 6, 7, 8, or 9 nucleotides at position 2, 5, 6, 8, 10, 14, 16, 17, and/or 18 from the 5′ end of the second nucleotide sequence is a 2′-fluoro nucleotide. In some embodiments, the nucleotide at position 2, 5, 6, 8, 10, 14, 16, 17, and/or 18 from the 5′ end of the second nucleotide sequence is a 2′-fluoro nucleotide. In some embodiments, at least two nucleotides at positions 2, 5, 6, 8, 10, 14, 16, 17, and/or 18 from the 5′ end of the second nucleotide sequence are 2′-fluoro nucleotides. In some embodiments, at least three nucleotides at positions 2, 5, 6, 8, 10, 14, 16, 17, and/or 18 from the 5′ end of the second nucleotide sequence are 2′-fluoro nucleotides. In some embodiments, at least four nucleotides at positions 2, 5, 6, 8, 10, 14, 16, 17, and/or 18 from the 5′ end of the second nucleotide sequence are 2′-fluoro nucleotides. In some embodiments, at least five nucleotides at positions 2, 5, 6, 8, 10, 14, 16, 17, and/or 18 from the 5′ end of the second nucleotide sequence are 2′-fluoro nucleotides. In some embodiments, the nucleotides at positions 2 and/or 14 from the 5′ end of the second nucleotide sequence are 2′-fluoro nucleotides. In some embodiments, the nucleotides at positions 2, 6, and/or 16 from the 5′ end of the second nucleotide sequence are 2′-fluoro nucleotides. In some embodiments, the nucleotides at positions 2, 6, 14, and/or 16 from the 5′ end of the second nucleotide sequence are 2′-fluoro nucleotides. In some embodiments, the nucleotides at positions 2, 6, 10, 14, and/or 18 from the 5′ end of the second nucleotide sequence are 2′-fluoro nucleotides. In some embodiments, the nucleotides at positions 2, 5, 8, 14, and/or 17 from the 5′ end of the second nucleotide sequence are 2′-fluoro nucleotides. In some embodiments, the nucleotide at position 2 from the 5′ end of the second nucleotide sequence is a 2′-fluoro nucleotide. In some embodiments, the nucleotide at position 5 from the 5′ end of the second nucleotide sequence is a 2′-fluoro nucleotide. In some embodiments, the nucleotide at position 6 from the 5′ end of the second nucleotide sequence is a 2′-fluoro nucleotide. In some embodiments, the nucleotide at position 8 from the 5′ end of the second nucleotide sequence is a 2′-fluoro nucleotide. In some embodiments, the nucleotide at position 10 from the 5′ end of the second nucleotide sequence is a 2′-fluoro nucleotide. In some embodiments, the nucleotide at position 14 from the 5′ end of the second nucleotide sequence is a 2′-fluoro nucleotide. In some embodiments, the nucleotide at position 16 from the 5′ end of the second nucleotide sequence is a 2′-fluoro nucleotide. In some embodiments, the nucleotide at position 17 from the 5′ end of the second nucleotide sequence is a 2′-fluoro nucleotide. In some embodiments, the nucleotide at position 18 from the 5′ end of the second nucleotide sequence is a 2′-fluoro nucleotide. In some embodiments, the 2′-fluoro nucleotide is a 2′-fluoro nucleotide mimic. The 2′-fluoro nucleotide mimic can be selected from

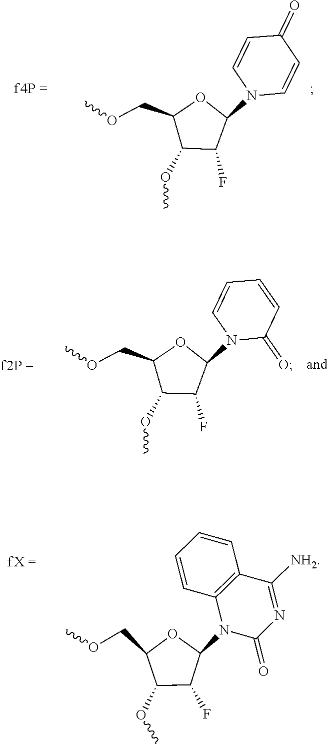

In some embodiments, the nucleotides in the second nucleotide sequence are arranged in an alternating 1:3 modification pattern, wherein 1 nucleotide is a 2′-fluoro nucleotide and 3 nucleotides are 2′-O-methyl nucleotides, and wherein the alternating 1:3 modification pattern occurs at least 2 times. In some embodiments, the alternating 1:3 modification pattern occurs 2-5 times. In some embodiments, at least two of the alternating 1:3 modification pattern occur consecutively. In some embodiments, at least two of the alternating 1:3 modification pattern occurs nonconsecutively. In some embodiments, at least 1, 2, 3, 4, or 5 alternating 1:3 modification pattern begins at nucleotide position 2, 6, 10, 14, and/or 18 from the 5′ end of the antisense strand. In some embodiments, at least one alternating 1:3 modification pattern begins at nucleotide position 2 from the 5′ end of the antisense strand. In some embodiments, wherein at least one alternating 1:3 modification pattern begins at nucleotide position 6 from the 5′ end of the antisense strand. In some embodiments, at least one alternating 1:3 modification pattern begins at nucleotide position 10 from the 5′ end of the antisense strand. In some embodiments, at least one alternating 1:3 modification pattern begins at nucleotide position 14 from the 5′ end of the antisense strand. In some embodiments, at least one alternating 1:3 modification pattern begins at nucleotide position 18 from the 5′ end of the antisense strand. In some embodiments, the 2′-fluoro nucleotide is a 2′-fluoro nucleotide mimic. The 2′-fluoro nucleotide mimic can be selected from

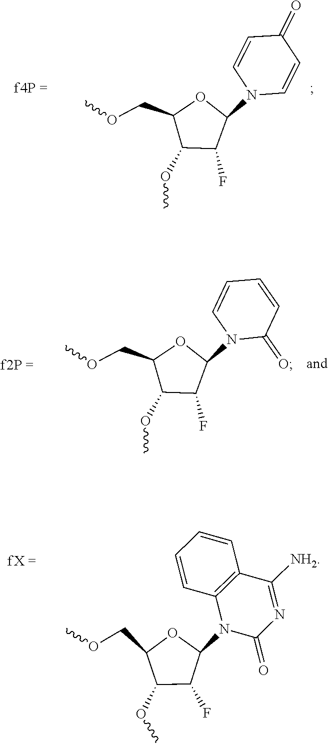

In some embodiments, the nucleotides in the second nucleotide sequence are arranged in an alternating 1:2 modification pattern, wherein 1 nucleotide is a 2′-fluoro nucleotide and 2 nucleotides are 2′-O-methyl nucleotides, and wherein the alternating 1:2 modification pattern occurs at least 2 times. In some embodiments, the alternating 1:2 modification pattern occurs 2-5 times. In some embodiments, at least two of the alternating 1:2 modification pattern occurs consecutively. In some embodiments, at least two of the alternating 1:2 modification pattern occurs nonconsecutively. In some embodiments, at least 1, 2, 3, 4, or 5 alternating 1:2 modification pattern begins at nucleotide position 2, 5, 8, 14, and/or 17 from the 5′ end of the antisense strand. In some embodiments, at least one alternating 1:2 modification pattern begins at nucleotide position 2 from the 5′ end of the antisense strand. In some embodiments, at least one alternating 1:2 modification pattern begins at nucleotide position 5 from the 5′ end of the antisense strand. In some embodiments, at least one alternating 1:2 modification pattern begins at nucleotide position 8 from the 5′ end of the antisense strand. In some embodiments, at least one alternating 1:2 modification pattern begins at nucleotide position 14 from the 5′ end of the antisense strand. In some embodiments, at least one alternating 1:2 modification pattern begins at nucleotide position 17 from the 5′ end of the antisense strand. In some embodiments, the 2′-fluoro nucleotide is a 2′-fluoro nucleotide mimic. The 2′-fluoro nucleotide mimic can be selected from

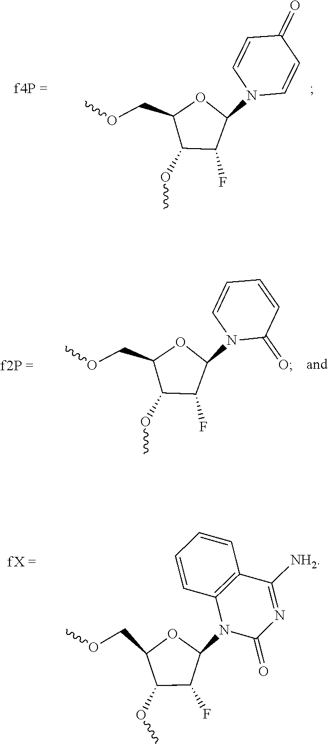

In some embodiments, the second nucleotide sequence comprises, consists of, or consists essentially of ribonucleic acids (RNAs). In some embodiments, the second nucleotide sequence comprises, consists of, or consists essentially of modified RNAs. In some embodiments, the modified RNAs are selected from a 2′-O-methyl RNA and 2′-fluoro RNA. In some embodiments, 15, 16, 17, 18, 19, 20, 21, 22, or 23 modified nucleotides of the second nucleotide sequence are independently selected from 2′-O-methyl RNA and 2′-fluoro RNA. In some embodiments, the 2′-fluoro nucleotide is a 2′-fluoro nucleotide mimic. The 2′-fluoro nucleotide mimic can be selected from

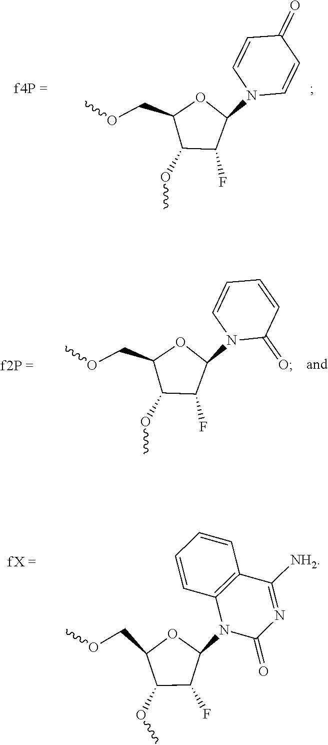

In some embodiments, the sense strand may further comprise one or more internucleotide linkages independently selected from a phosphodiester (PO) internucleoside linkage, phosphorothioate (PS) internucleoside linkage, phosphorodithioate internucleoside linkage, and PS-mimic internucleoside linkage. In some embodiments, the PS-mimic internucleoside linkage is a sulfo internucleotide linkage.

In some embodiments, the antisense strand may further comprise at least 1, 2, 3, 4, 5, 6, 7, 8, 9, 10, 11, 12, 13, 14, or 15 or more phosphorothioate internucleoside linkages. In some embodiments, the antisense strand comprises 20, 19, 18, 17, 16, 15, 14, 13, 12, 11, 10, 9, 8, 7, 6, 5, 4, or 3 or fewer phosphorothioate internucleoside linkages. In some embodiments, the antisense strand comprises 2 to 10, 2 to 8, 2 to 6, 1 to 5, 1 to 4, 1 to 3, or 1 to 2 phosphorothioate internucleoside linkages. In some embodiments, the antisense strand comprises 2 to 10, 2 to 8, 2 to 6, 1 to 5, 1 to 4, 1 to 3, or 1 to 2 phosphorothioate internucleoside linkages. In some embodiments, the antisense strand comprises 2 to 8 phosphorothioate internucleoside linkages. In some embodiments, the antisense strand comprises 3 to 8 phosphorothioate internucleoside linkages. In some embodiments, the antisense strand comprises 4 to 8 phosphorothioate internucleoside linkages. In some embodiments, at least one phosphorothioate internucleoside linkage is between the nucleotides at positions 1 and 2 from the 5′ end of the second nucleotide sequence. In some embodiments, at least one phosphorothioate internucleoside linkage is between the nucleotides at positions 2 and 3 from the 5′ end of the second nucleotide sequence.

In some embodiments, at least one phosphorothioate internucleoside linkage is between the nucleotides at positions 1 and 2 from the 3′ end of the second nucleotide sequence. In some embodiments, at least one phosphorothioate internucleoside linkage is between the nucleotides at positions 2 and 3 from the 3′ end of the second nucleotide sequence. In some embodiments, the antisense strand comprises two phosphorothioate internucleoside linkages between the nucleotides at positions 1 to 3 from the 5′ end of the first nucleotide sequence. In some embodiments, the antisense strand comprises two phosphorothioate internucleoside linkages between the nucleotides at positions 1 to 3 from the 3′ end of the first nucleotide sequence. In some embodiments, the antisense strand comprises (a) two phosphorothioate internucleoside linkages between the nucleotides at positions 1 to 3 from the 5′ end of the first nucleotide sequence; and (b) two phosphorothioate internucleoside linkages between the nucleotides at positions 1 to 3 from the 3′ end of the first nucleotide sequence.

In some embodiments, at least one end of the ds-siNA is a blunt end. In some embodiments, at least one end of the ds-siNA comprises an overhang, wherein the overhang comprises at least one nucleotide. In some embodiments, both ends of the ds-siNA comprise an overhang, wherein the overhang comprises at least one nucleotide. In some embodiments, the overhang comprises 1 to 5 nucleotides, 1 to 4 nucleotides, 1 to 3 nucleotides, or 1 to 2 nucleotides. In some embodiments, the overhang consists of 1 to 2 nucleotides.

In some embodiments, any of the antisense strands disclosed herein may comprise a 5′ end cap monomer. In some embodiments, any of the second nucleotide sequences disclosed herein may comprise a 5′ end cap monomer.

### Modified Nucleotides

Further disclosed herein are siNA molecules comprising one or more modified nucleotides. In some embodiments, any of the siNAs disclosed herein comprise one or more modified nucleotides. In some embodiments, any of the sense strands disclosed herein comprise one or more modified nucleotides. In some embodiments, any of the first nucleotide sequences disclosed herein comprise one or more modified nucleotides. In some embodiments, any of the antisense strands disclosed herein comprise one or more modified nucleotides. In some embodiments, any of the second nucleotide sequences disclosed herein comprise one or more modified nucleotides. In some embodiments, the one or more modified nucleotides is adjacent to the first nucleotide sequence. In some embodiments, at least one modified nucleotide is adjacent to the 5′ end of the first nucleotide sequence. In some embodiments, at least one modified nucleotide is adjacent to the 3′ end of the first nucleotide sequence. In some embodiments, at least one modified nucleotide is adjacent to the 5′ end of the first nucleotide sequence and at least one modified nucleotide is adjacent to the 3′ end of the first nucleotide sequence. In some embodiments, the one or more modified nucleotides is adjacent to the second nucleotide sequence. In some embodiments, at least one modified nucleotide is adjacent to the 5′ end of the second nucleotide sequence. In some embodiments, at least one modified nucleotide is adjacent to the 3′ end of the second nucleotide sequence. In some embodiments, at least one modified nucleotide is adjacent to the 5′ end of the second nucleotide sequence and at least one modified nucleotide is adjacent to the 3′ end of the second nucleotide sequence. In some embodiments, a 2′-O-methyl nucleotide in any of sense strands or first nucleotide sequences disclosed herein is replaced with a modified nucleotide. In some embodiments, a 2′-O-methyl nucleotide in any of antisense strands or second nucleotide sequences disclosed herein is replaced with a modified nucleotide.

In some embodiments, any of the siNA molecules, siNAs, sense strands, first nucleotide sequences, antisense strands, and second nucleotide sequences disclosed herein comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 or more modified nucleotides. In some embodiments, 1%, 2%, 3%, 4%, 5%, 10%, 15%, 20%, 25%, 30%, 35%, 40%, 45%, 50%, 55%, 60%, 70%, 75%, 80%, 85%, 86%, 87%, 88%, 90%, 91%, 92%, 93%, 94%, 95%, 96%, 97%, 98%, 99% or 100% of the nucleotides in the siNA molecule, siNA, sense strand, first nucleotide sequence, antisense strand, or second nucleotide sequence are modified nucleotides.

In some embodiments, a modified nucleotide is selected from the group consisting of 2′-fluoro nucleotide, 2′-O-methyl nucleotide, 2′-fluoro nucleotide mimic, 2′-O-methyl nucleotide mimic, a locked nucleic acid, and a nucleotide comprising a modified nucleobase.

In some embodiments, any of the siRNAs disclosed herein comprise at least 1, 2, 3, 4, 5, 6, 7, 8, 9, or 10 or more 2′-fluoro or 2′-O-methyl nucleotide mimics. In some embodiments, any of the sense strands disclosed herein comprise at least 1, 2, 3, 4, 5, 6, 7, 8, 9, or 10 or more 2′-fluoro or 2′-O-methyl nucleotide mimics. In some embodiments, any of the first nucleotide sequences disclosed herein comprise at least 1, 2, 3, 4, 5, 6, 7, 8, 9, or 10 or more 2′-fluoro or 2′-O-methyl nucleotide mimics. In some embodiments, any of the antisense strand disclosed herein comprise at least 1, 2, 3, 4, 5, 6, 7, 8, 9, or 10 or more 2′-fluoro or 2′-O-methyl nucleotide mimics. In some embodiments, any of the second nucleotide sequences disclosed herein comprise at least 1, 2, 3, 4, 5, 6, 7, 8, 9, or 10 or more 2′-fluoro or 2′-O-methyl nucleotide mimics. In some embodiments, the 2′-fluoro or 2′-O-methyl nucleotide mimic is a nucleotide mimic of Formula (16)-Formula (20):

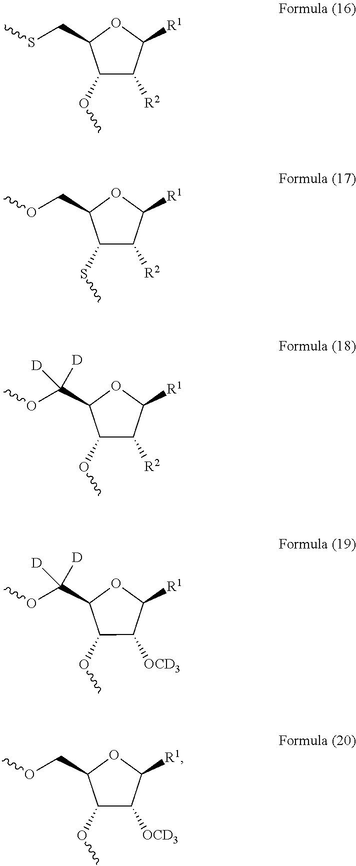

wherein R¹ is a nucleobase and R¹ is independently F or —OCH₃. In some embodiments, the nucleobase is selected from cytosine, guanine, adenine, uracil, aryl, heteroaryl, and an analogue or derivative thereof.

In some embodiments, the siNA molecules disclosed herein comprise at least one 2′-fluoro nucleotide, at least one 2′-O-methyl nucleotide, and at least one 2′-fluoro or 2′-O-methyl nucleotide mimic. In some embodiments, the at least one 2′-fluoro or 2′-O-methyl nucleotide mimic is adjacent to the first nucleotide sequence. In some embodiments, the at least one 2′-fluoro or 2′-O-methyl nucleotide mimic is adjacent to the 5′ end of first nucleotide sequence. In some embodiments, the at least one 2′-fluoro or 2′-O-methyl nucleotide mimic is adjacent to the 3′ end of first nucleotide sequence. In some embodiments, the at least one 2′-fluoro or 2′-O-methyl nucleotide mimic is adjacent to the second nucleotide sequence. In some embodiments, the at least one 2′-fluoro or 2′-O-methyl nucleotide mimic is adjacent to the 5′ end of second nucleotide sequence. In some embodiments, the at least one 2′-fluoro or 2′-O-methyl nucleotide mimic is adjacent to the 3′ end of second nucleotide sequence. In some embodiments, the first nucleotide sequence does not comprise a 2′-fluoro nucleotide mimic. In some embodiments, the first nucleotide sequence does not comprise a 2′-O-methyl nucleotide mimic. In some embodiments, the second nucleotide sequence does not comprise a 2′-fluoro nucleotide mimic. In some embodiments, the second nucleotide sequence does not comprise a 2′-O-methyl nucleotide mimic.

In some embodiments, any of the siRNAs disclosed herein comprise at least 1, 2, 3, 4, 5, 6, 7, 8, 9, or 10 or more locked nucleic acids. In some embodiments, any of the sense strands disclosed herein comprise at least 1, 2, 3, 4, 5, 6, 7, 8, 9, or 10 or more locked nucleic acids. In some embodiments, any of the first nucleotide sequences disclosed herein comprise at least 1, 2, 3, 4, 5, 6, 7, 8, 9, or 10 or more locked nucleic acids. In some embodiments, any of the antisense strand disclosed herein comprise at least 1, 2, 3, 4, 5, 6, 7, 8, 9, or 10 or more locked nucleic acids. In some embodiments, any of the second nucleotide sequences disclosed herein comprise at least 1, 2, 3, 4, 5, 6, 7, 8, 9, or 10 or more locked nucleic acids. In some embodiments, the locked nucleic acid is selected from

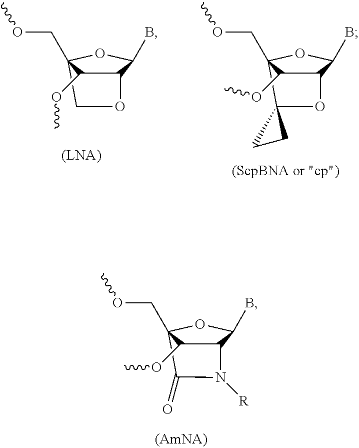

where R is H or alkyl (or AmNA(N-Me)) when R is alkyl);

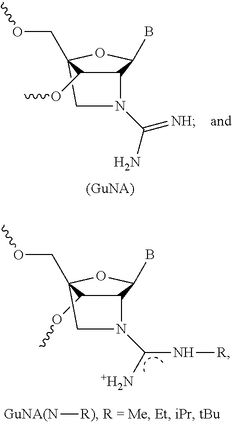

wherein B is a nucleobase. In some embodiments, any of the siRNAs, sense strands, first nucleotide sequences, antisense strands, or second nucleotide sequences disclosed herein comprise at least modified nucleotide that is

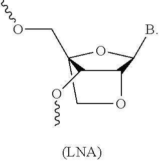

In some embodiments, any of the siRNAs, sense strands, first nucleotide sequences, antisense strands, or second nucleotide sequences disclosed herein comprise at least modified nucleotide that is

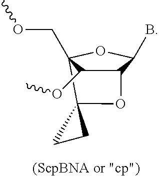

In some embodiments, any of the siRNAs, sense strands, first nucleotide sequences, antisense strands, or second nucleotide sequences disclosed herein comprise at least modified nucleotide that is

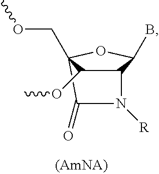

where R is H or alkyl (or AmNA(N-Me)) when R is alkyl). In some embodiments, any of the siRNAs, sense strands, first nucleotide sequences, antisense strands, or second nucleotide sequences disclosed herein comprise at least modified nucleotide that is

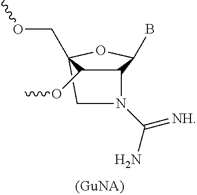

In some embodiments, any of the siRNAs, sense strands, first nucleotide sequences, antisense strands, or second nucleotide sequences disclosed herein comprise at least modified nucleotide that is

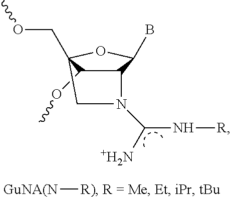

wherein B is a nucleobase.

### Phosphorylation Blocker

Further disclosed herein are siNA molecules comprising a phosphorylation blocker. In some embodiments, a 2′-O-methyl nucleotide in any of sense strands or first nucleotide sequences disclosed herein is replaced with a nucleotide containing a phosphorylation blocker. In some embodiments, a 2′-O-methyl nucleotide in any of antisense strands or second nucleotide sequences disclosed herein is replaced with a nucleotide containing a phosphorylation blocker. In some embodiments, a 2′-O-methyl nucleotide in any of sense strands or first nucleotide sequences disclosed herein is further modified to contain a phosphorylation blocker. In some embodiments, a 2′-O-methyl nucleotide in any of antisense strands or second nucleotide sequences disclosed herein is further modified to contain a phosphorylation blocker.

In some embodiments, any of the siNA molecules disclosed herein comprise a phosphorylation blocker of Formula (IV):

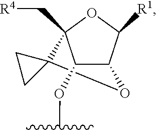

wherein R¹ is a nucleobase, R⁴ is —O—R³⁰ or —NR³¹R³², R³⁰ is C₁-C₈ substituted or unsubstituted alkyl; and R³¹ and R³² together with the nitrogen to which they are attached form a substituted or unsubstituted heterocyclic ring.

In some embodiments, any of the siNA molecules disclosed herein comprise a phosphorylation blocker of Formula (IV):

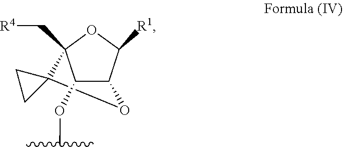

wherein R¹ is a nucleobase, and R⁴ is —OCH₃ or —N(CH₂CH₂)₂O.

In some embodiments, a siNA molecule comprises (a) a phosphorylation blocker of Formula (IV):

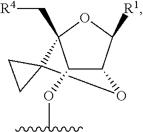

wherein R¹ is a nucleobase, R⁴ is —O—R³⁰ or —NR³¹R³², R³⁰ is C₁-C₈ substituted or unsubstituted alkyl; and R³¹ and R³² together with the nitrogen to which they are attached form a substituted or unsubstituted heterocyclic ring; and (b) a siNA, wherein the phosphorylation blocker is conjugated to the siNA.

In some embodiments, a siNA molecule comprises (a) a phosphorylation blocker of Formula (IV):

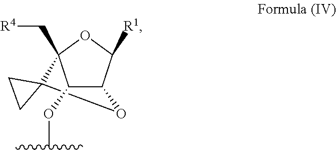

wherein R¹ is a nucleobase, and R⁴ is —OCH₃ or —N(CH₂CH₂)₂O; and (b) siNA, wherein the phosphorylation blocker is conjugated to the siNA.

In some embodiments, the phosphorylation blocker is attached to the 3′ end of the sense strand or first nucleotide sequence. In some embodiments, the phosphorylation blocker is attached to the 3′ end of the sense strand or first nucleotide sequence via 1, 2, 3, 4, or 5 or more linkers. In some embodiments, the phosphorylation blocker is attached to the 5′ end of the sense strand or first nucleotide sequence. In some embodiments, the phosphorylation blocker is attached to the 5′ end of the sense strand or first nucleotide sequence via 1, 2, 3, 4, or 5 or more linkers. In some embodiments, the phosphorylation blocker is attached to the 3′ end of the antisense strand or second nucleotide sequence. In some embodiments, the phosphorylation blocker is attached to the 3′ end of the antisense strand or second nucleotide sequence via 1, 2, 3, 4, or 5 or more linkers. In some embodiments, the phosphorylation blocker is attached to the 5′ end of the antisense strand or second nucleotide sequence. In some embodiments, the phosphorylation blocker is attached to the 5′ end of the antisense strand or second nucleotide sequence via 1, 2, 3, 4, or 5 or more linkers. In some embodiments, the one or more linkers are independently selected from the group consisting of a phosphodiester linker, phosphorothioate linker, and phosphorodithioate linker.

### 5′-Stabilized End Cap

Further disclosed herein are siNA molecules comprising a 5′-stabilized end cap. As used herein the terms “5′-stabilized end cap” and “5′ end cap” are used interchangeably. In some embodiments, a 2′-O-methyl nucleotide in any of sense strands or first nucleotide sequences disclosed herein is replaced with a nucleotide containing a 5′-stabilized end cap. In some embodiments, a 2′-O-methyl nucleotide in any of antisense strands or second nucleotide sequences disclosed herein is replaced with a nucleotide containing a 5′-stabilized end cap. In some embodiments, a 2′-O-methyl nucleotide in any of sense strands or first nucleotide sequences disclosed herein is further modified to contain a 5′-stabilized end cap. In some embodiments, a 2′-O-methyl nucleotide in any of antisense strands or second nucleotide sequences disclosed herein is further modified to contain a 5′-stabilized end cap.

In some embodiments, the 5′-stabilized end cap is a 5′ phosphate mimic. In some embodiments, the 5′-stabilized end cap is a modified 5′ phosphate mimic. In some embodiments, the modified 5′ phosphate is a chemically modified 5′ phosphate. In some embodiments, the 5′-stabilized end cap is a 5′-vinyl phosphonate. In some embodiments, the 5′-vinyl phosphonate is a 5′-(E)-vinyl phosphonate or 5′-(Z)-vinyl phosphonate. In some embodiments, the 5′-vinylphosphonate is a deuterated vinyl phosphonate. In some embodiments, the deuterated vinyl phosphonate is a mono-deuterated vinyl phosphonate. In some embodiments, the deuterated vinyl phosphonate is a di-deuterated vinyl phosphonate. In some embodiments, the 5′-stabilized end cap is a phosphate mimic. Examples of phosphate mimics are disclosed in Parmar et al., 2018, J Med Chem, 61(3):734-744, International Publication Nos. WO2018/045317 and WO2018/044350, and U.S. Pat. No. 10,087,210, each of which is incorporated by reference in its entirety.

In some embodiments, any of the siNA molecules, sense strands, first nucleotide sequences, antisense strands, or second nucleotide sequences disclosed herein comprise a 5′-stabilized end cap of Formula (Ia):

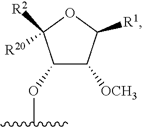

wherein R¹ is H, a nucleobase, aryl, or heteroaryl; R² is

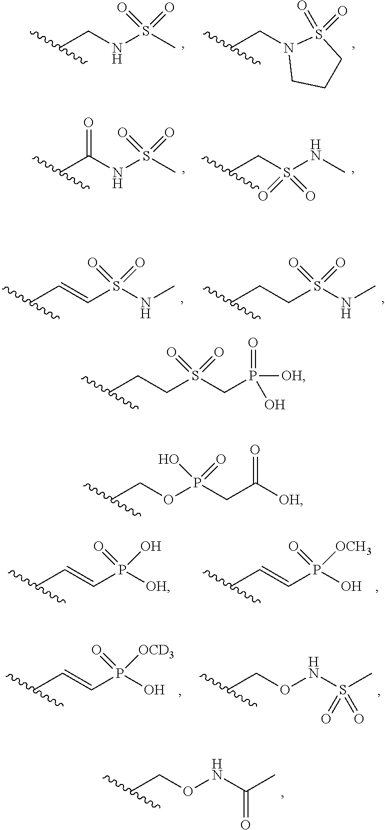

—CH═CD-Z, —CD=CH—Z, —CD=CD-Z, —(CR²¹R²²)ₙ—Z, or —(C₂-C₆ alkenylene)-Z and R²⁰ is H; or R² and R²⁰ together form a 3- to 7-membered carbocyclic ring substituted with —(CR²¹R²²)ₙ—Z or —(C₂-C₆ alkenylene)-Z; n is 1, 2, 3, or 4; Z is —ONR²³R²⁴, —OP(O)OH(CH₂)ₘCO₂R²³, —OP(S)OH(CH₂)ₘCO₂R²³, —P(O)(OH)₂, —P(O)(OH)(OCH₃), —P(O)(OH)(OCD₃), —SO₂(CH₂)ₘP(O)(OH)₂, —SO₂NR²³R²⁵, —NR²³R²⁴, —NR²³ SO₂R²⁵; either R²¹ and R²² are independently hydrogen or C₁-C₆ alkyl, or R²¹ and R²² together form an oxo group; R²³ is hydrogen or C₁-C₆ alkyl; R²⁴ is —SO₂R²⁵ or —C(O)R²⁵; or R²³ and R²⁴ together with the nitrogen to which they are attached form a substituted or unsubstituted heterocyclic ring; R²⁵ is C₁-C₆ alkyl; and m is 1, 2, 3, or 4. In some embodiments, R¹ is an aryl. In some embodiments, the aryl is a phenyl.

In some embodiments, any of the siNA molecules, sense strands, first nucleotide sequences, antisense strands, or second nucleotide sequences disclosed herein comprise a 5′-stabilized end cap of Formula (Ib):

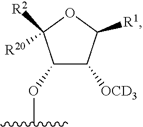

wherein R¹ is H, a nucleobase, aryl, or heteroaryl; R² is

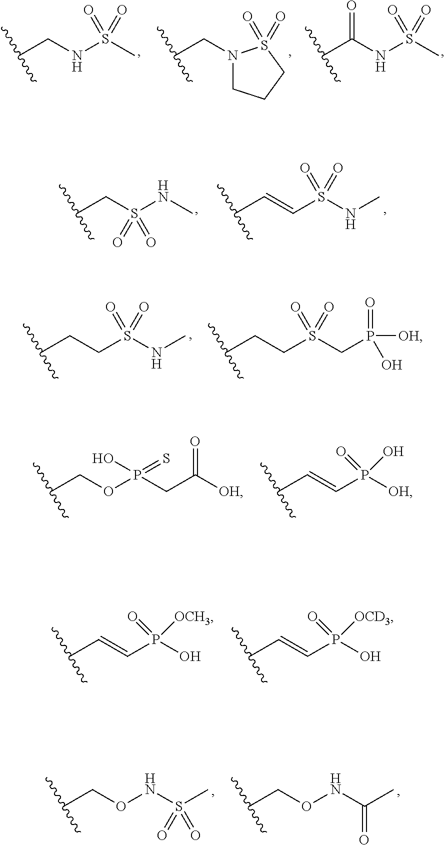

—CH═CD-Z, —CD=CH—Z, —CD=CD-Z, —(CR²¹R²²)ₙ—Z, or —(C₂-C₆ alkenylene)-Z and R²⁰ is H; or R² and R²⁰ together form a 3- to 7-membered carbocyclic ring substituted with —(CR²¹R²²)ₙ—Z or —(C₂-C₆ alkenylene)-Z; n is 1, 2, 3, or 4; Z is —ONR²³R²⁴, —OP(O)OH(CH₂)ₘCO₂R²³, —OP(S)OH(CH₂)ₘCO₂R²³, —P(O)(OH)₂, —P(O)(OH)(OCH₃), —P(O)(OH)(OCD₃), —SO₂(CH₂)ₘP(O)(OH)₂, —SO₂NR²³R²⁵, —NR²³R²⁴, —NR²³SO₂R²⁵; either R²¹ and R²² are independently hydrogen or C₁-C₆ alkyl, or R²¹ and R²² together form an oxo group; R²³ is hydrogen or C₁-C₆ alkyl; R²⁴ is —SO₂R²⁵ or —C(O)R²⁵; or R²³ and R²⁴ together with the nitrogen to which they are attached form a substituted or unsubstituted heterocyclic ring; R²⁵ is C₁-C₆ alkyl; and m is 1, 2, 3, or 4. In some embodiments, R¹ is an aryl. In some embodiments, the aryl is a phenyl.

In some embodiments, any of the siNA molecules, sense strands, first nucleotide sequences, antisense strands, or second nucleotide sequences disclosed herein comprise a 5′-stabilized end cap of Formula (Ic):

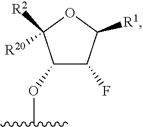

wherein R¹ is a nucleobase, aryl, heteroaryl, or H,

### R² is

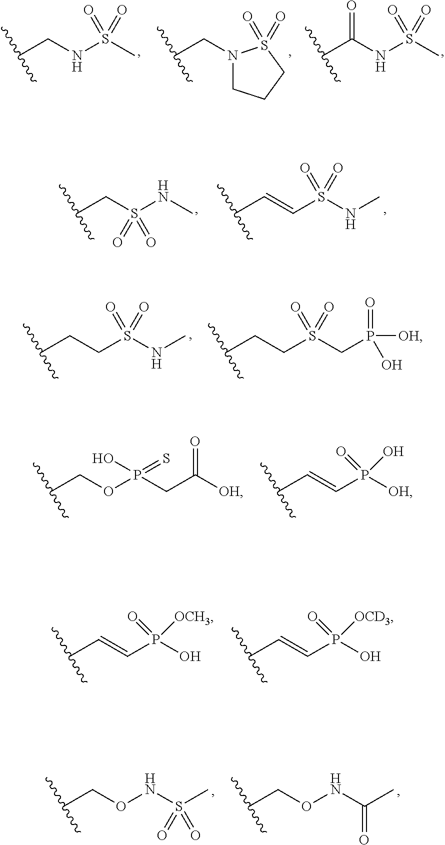

—CH═CD-Z, —CD=CH—Z, —CD=CD-Z, —(CR²¹R²²)ₙ—Z, or —(C₂-C₆ alkenylene)-Z and R²⁰ is hydrogen; or R² and R²⁰ together form a 3- to 7-membered carbocyclic ring substituted with —(CR²¹R²²)ₙ—Z or —(C₂-C₆ alkenylene)-Z; n is 1, 2, 3, or 4; Z is —ONR²³R²⁴, —OP(O)OH(CH₂)ₘCO₂R²³, —OP(S)OH(CH₂)ₘCO₂R²³, —P(O)(OH)₂, —P(O)(OH)(OCH₃), —P(O)(OH)(OCD₃), —SO₂(CH₂)ₘP(O)(OH)₂, —SO₂NR²³R²⁵, —NR²³R²⁴, or —NR²³SO₂R²⁵; R²¹ and R²² either are independently hydrogen or C₁-C₆ alkyl, or R²¹ and R²² together form an oxo group; R²³ is hydrogen or C₁-C₆ alkyl; R²⁴ is —SO₂R²⁵ or —C(O)R²⁵; or

R²³ and R²⁴ together with the nitrogen to which they are attached form a substituted or unsubstituted heterocyclic ring; R²⁵ is C₁-C₆ alkyl; and m is 1, 2, 3, or 4. In some embodiments, R¹ is an aryl. In some embodiments, the aryl is a phenyl.

In some embodiments, any of the siNA molecules, sense strands, first nucleotide sequences, antisense strands, or second nucleotide sequences disclosed herein comprise a 5′-stabilized end cap of Formula (IIa):

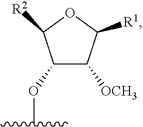

wherein R¹ is a nucleobase, aryl, heteroaryl,

### or H, R² is

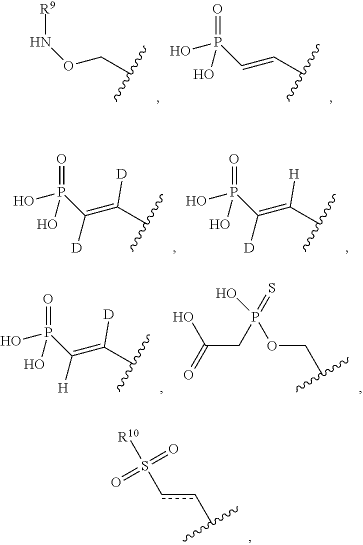

—CH₂SO₂NHCH₃, or

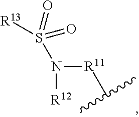

R⁹ is —SO₂CH₃ or —COCH₃, [symbol] is a double or single bond, R¹⁰=—CH₂PO₃H or —NHCH₃, R¹¹ is —CH₂— or —CO—, and R¹² is H and R¹¹ is CH₃ or R¹² and R¹¹ together form —CH₂CH₂CH₂—. In some embodiments, le is an aryl. In some embodiments, the aryl is a phenyl.

In some embodiments, any of the siNA molecules, sense strands, first nucleotide sequences, antisense strands, or second nucleotide sequences disclosed herein comprise a 5′-stabilized end cap of Formula (IIb):

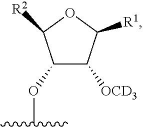

wherein R¹ is a nucleobase, aryl, heteroaryl, or H, R² is

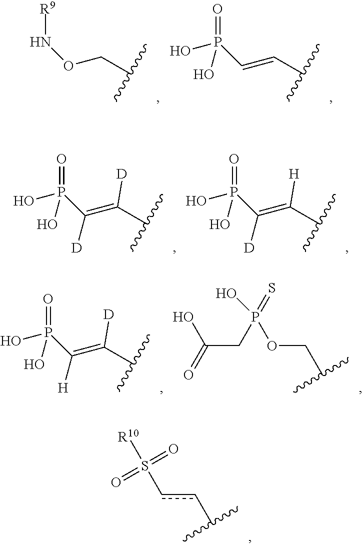

—CH₂SO₂NHCH₃, or

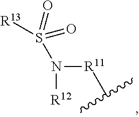

R⁹ is —SO₂CH₃ or —COCH₃, [symbol] is a double or single bond, R¹⁰=—CH₂PO₃H or —NHCH₃, is —CH₂— or —CO—, and R¹² is H and R¹³ is CH₃ or R¹² and R¹³ together form —CH₂CH₂CH₂—. In some embodiments, le is an aryl. In some embodiments, the aryl is a phenyl.

In some embodiments, any of the siNA molecules, sense strands, first nucleotide sequences, antisense strands, or second nucleotide sequences disclosed herein comprise a 5′-stabilized end cap of Formula (III):

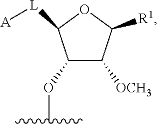

wherein R¹ is a nucleobase, aryl, heteroaryl, or H, L is —CH₂—, —CH═CH—, —CO—, or —CH₂CH₂—, and A is —ONHCOCH₃, —ONHSO₂CH₃, —PO₃H, —OP(SOH)CH₂CO₂H, —SO₂CH₂PO₃H, —SO₂NHCH₃, —NHSO₂CH₃, or —N(SO₂CH₂CH₂CH₂). In some embodiments, R¹ is an aryl. In some embodiments, the aryl is a phenyl.

In some embodiments, any of the siNA molecules, sense strands, first nucleotide sequences, antisense strands, or second nucleotide sequences disclosed herein comprise a 5′-stabilized end cap selected from Examples 5-11, 33-35, 38, 39, 43, and 49-53 5′ end cap monomers.

Further disclosed herein are siNA molecules comprising (a) a 5′-stabilized end cap of Formula (Ia):

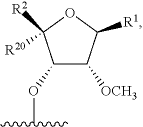

wherein R¹ is a nucleobase, aryl, heteroaryl, or H; R² is

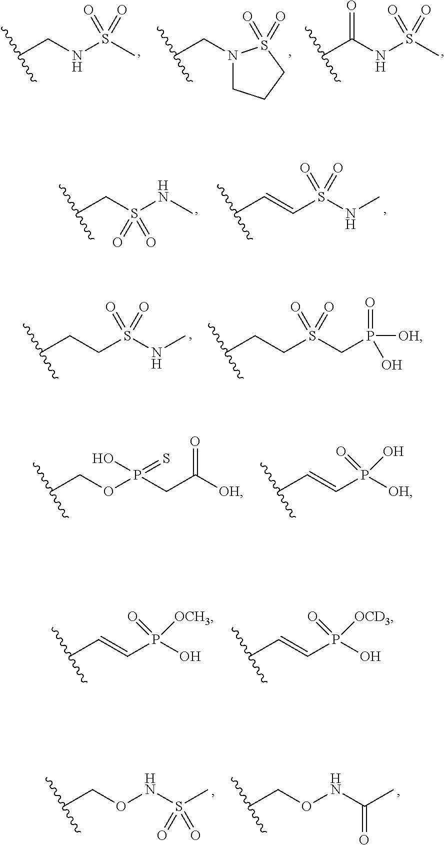

—CH═CD-Z, —CD=CH—Z, —CD=CD-Z, —(CR²¹R²²)ₙ—Z, or —(C₂-C₆ alkenylene)-Z and R²⁰ is H; or R² and R²⁰ together form a 3- to 7-membered carbocyclic ring substituted with —(CR²¹R²²)ₙ—Z or —(C₂-C₆ alkenylene)-Z; n is 1, 2, 3, or 4; Z is —ONR²³R²⁴, —OP(O)OH(CH₂)ₘCO₂R²³, —OP(S)OH(CH₂)ₘCO₂R²³, —P(O)(OH)₂, —P(O)(OH)(OCH₃), —P(O)(OH)(OCD₃), —SO₂(CH₂)ₘP(O)(OH)₂, —SO₂NR²³R²⁵, —NR²³R²⁴, —NR²³SO₂R²⁵; either R²¹ and R²² are independently hydrogen or C₁-C₆ alkyl, or R²¹ and R²² together form an oxo group; R²³ is hydrogen or C₁-C₆ alkyl; R²⁴ is —SO₂R²⁵ or —C(O)R²⁵; or R²³ and R²⁴ together with the nitrogen to which they are attached form a substituted or unsubstituted heterocyclic ring; R²⁵ is C₁-C₆ alkyl; and m is 1, 2, 3, or 4; and (b) a short interfering nucleic acid (siNA), wherein the 5′-stabilized end cap is conjugated to the siNA. In some embodiments, R¹ is an aryl. In some embodiments, the aryl is a phenyl.

Further disclosed herein are siNA molecules comprising (a) a 5′-stabilized end cap of Formula (Ib):

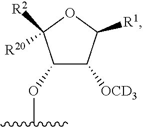

wherein R¹ is a nucleobase, aryl, heteroaryl, or H; R² is

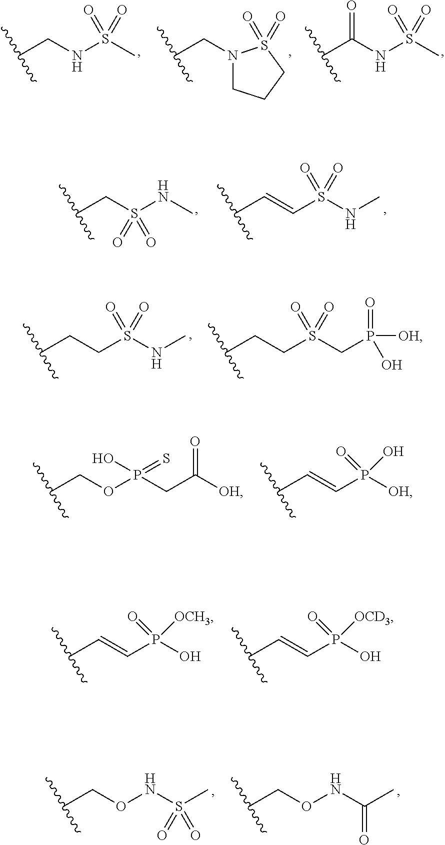

—CH═CD-Z, —CD=CH—Z, —CD=CD-Z, —(CR²¹R²²)ₙ—Z, or —(C₂-C₆ alkenylene)-Z and R²⁰ is H; or R² and R²⁰ together form a 3- to 7-membered carbocyclic ring substituted with —(CR²¹R²²)ₙ—Z or —(C₂-C₆ alkenylene)-Z; n is 1, 2, 3, or 4; Z is —ONR²³R²⁴, —OP(O)OH(CH₂)ₘCO₂R²³, —OP(S)OH(CH₂)ₘCO₂R²³, —P(O)(OH)₂, —P(O)(OH)(OCH₃), —P(O)(OH)(OCD₃), —SO₂(CH₂)ₘP(O)(OH)₂, —SO₂NR²³R²⁵, —NR²³R²⁴, —NR²³SO₂R²⁵; either R²¹ and R²² are independently hydrogen or C₁-C₆ alkyl, or R²¹ and R²² together form an oxo group; R²³ is hydrogen or C₁-C₆ alkyl; R²⁴ is —SO₂R²⁵ or —C(O)R²⁵; or R²³ and R²⁴ together with the nitrogen to which they are attached form a substituted or unsubstituted heterocyclic ring; R²⁵ is C₁-C₆ alkyl; and m is 1, 2, 3, or 4; and (b) a short interfering nucleic acid (siNA), wherein the 5′-stabilized end cap is conjugated to the siNA. In some embodiments, R¹ is an aryl. In some embodiments, the aryl is a phenyl.

Further disclosed herein are siNA molecules comprising (a) a 5′-stabilized end cap of Formula (Ic):

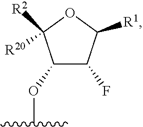

wherein R¹ is a nucleobase, aryl, heteroaryl, or H, R² is

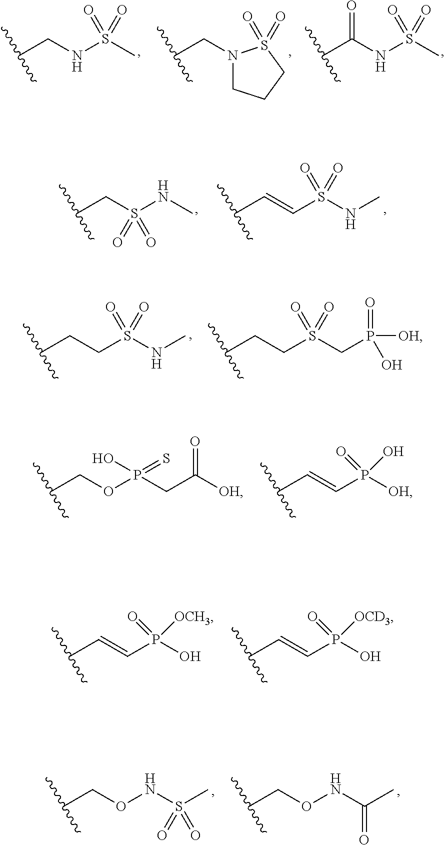

—CH═CD-Z, —CD=CH—Z, —CD=CD-Z, —(CR²¹R²²)ₙ—Z, or —(C₂-C₆ alkenylene)-Z and R²⁰ is hydrogen; or R² and R²⁰ together form a 3- to 7-membered carbocyclic ring substituted with —(CR²¹R²²)ₙ—Z or —(C₂-C₆ alkenylene)-Z; n is 1, 2, 3, or 4; Z is —ONR²³R²⁴, —OP(O)OH(CH₂)ₘCO₂R²³, —OP(S)OH(CH₂)ₘCO₂R²³, —P(O)(OH)₂, —P(O)(OH)(OCH₃), —P(O)(OH)(OCD₃), —SO₂(CH₂)ₘP(O)(OH)₂, —SO₂NR²³R²⁵, NR²³R²⁴, or —NR²³SO₂R²⁵; R²¹ and R²² either are independently hydrogen or C₁-C₆ alkyl, or R²¹ and R²² together form an oxo group; R²³ is hydrogen or C₁-C₆ alkyl; R²⁴ is —SO₂R²⁵ or —C(O)R²⁵; or R²³ and R²⁴ together with the nitrogen to which they are attached form a substituted or unsubstituted heterocyclic ring; R²⁵ is C₁-C₆ alkyl; and m is 1, 2, 3, or 4; and (b) a short interfering nucleic acid (siNA), wherein the 5′-stabilized end cap is conjugated to the siNA. In some embodiments, R¹ is an aryl. In some embodiments, the aryl is a phenyl.

In some embodiments, a siNA molecule comprises (a) a 5′-stabilized end cap of Formula (IIa):

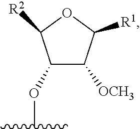

wherein R¹ is a nucleobase, aryl, heteroaryl, or H, R² is

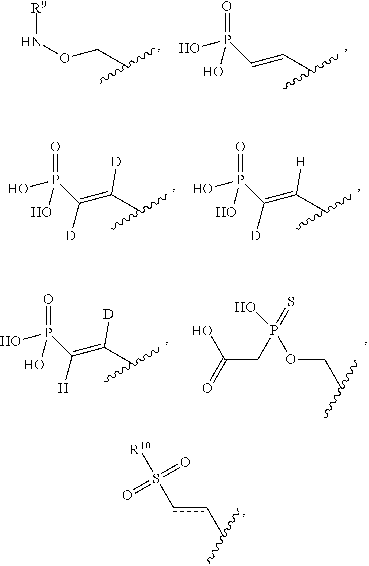

CH₂SO₂NHCH₃, or

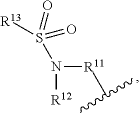

R⁹ is —SO₂CH₃ or —COCH₃, wherein [symbol] is a double or single bond, R¹⁰=—CH₂PO₃H or —NHCH₃, R¹¹ is —CH₂— or —CO—, and R¹² is H and R¹³ is CH₃ or R¹² and R¹³ together form —CH₂CH₂CH₂—; and (b) a short interfering nucleic acid (siNA), wherein the 5′-stabilized end cap is conjugated to the siNA. In some embodiments, R¹ is an aryl. In some embodiments, the aryl is a phenyl.

In some embodiments, a siNA molecule comprises (a) a 5′-stabilized end cap of Formula (IIb):

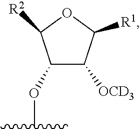

wherein R¹ is a nucleobase, aryl, heteroaryl, or H, R² is

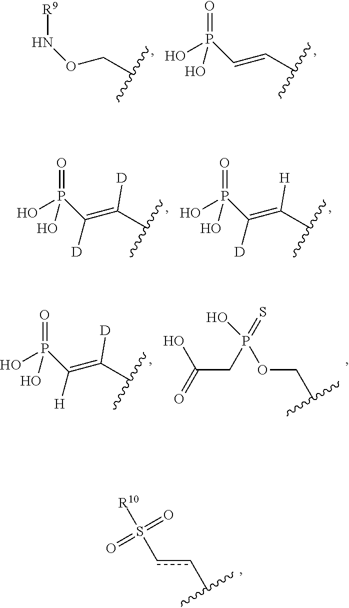

CH₂SO₂NHCH₃, or

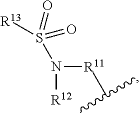

R⁹ is —SO₂CH₃ or —COCH₃, wherein [symbol] is a double or single bond, R¹⁰=—CH₂PO₃H or —NHCH₃, R¹¹ is —CH₂— or —CO—, and R¹² is H and R¹³ is CH₃ or R¹² and R¹³ together form —CH₂CH₂CH₂—; and (b) a short interfering nucleic acid (siNA), wherein the 5′-stabilized end cap is conjugated to the siNA. In some embodiments, R¹ is an aryl. In some embodiments, the aryl is a phenyl.

In some embodiments, a siNA molecule comprises (a) a 5′-stabilized end cap of Formula (III):

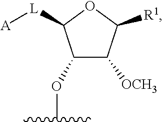

wherein R¹ is a nucleobase, aryl, heteroaryl, or H, L is —CH₂—, —CH═CH—, —CO—, or —CH₂CH₂—, and A is —ONHCOCH₃, —ONHSO₂CH₃, —PO₃H, —OP(SOH)CH₂CO₂H, —SO₂CH₂PO₃H, —SO₂NHCH₃, —NHSO₂CH₃, or —N(SO₂CH₂CH₂CH₂); and (b) a siNA, wherein the 5′-stabilized end cap is conjugated to the siNA. In some embodiments, 10 is an aryl. In some embodiments, the aryl is phenyl.

In some embodiments, any of the siNA molecules disclosed herein comprise a 5′-stabilized end cap selected from the group consisting of Formula (1) to Formula (15), Formula (9X) to Formula (12X), and Formula (9Y) to Formula (12Y):

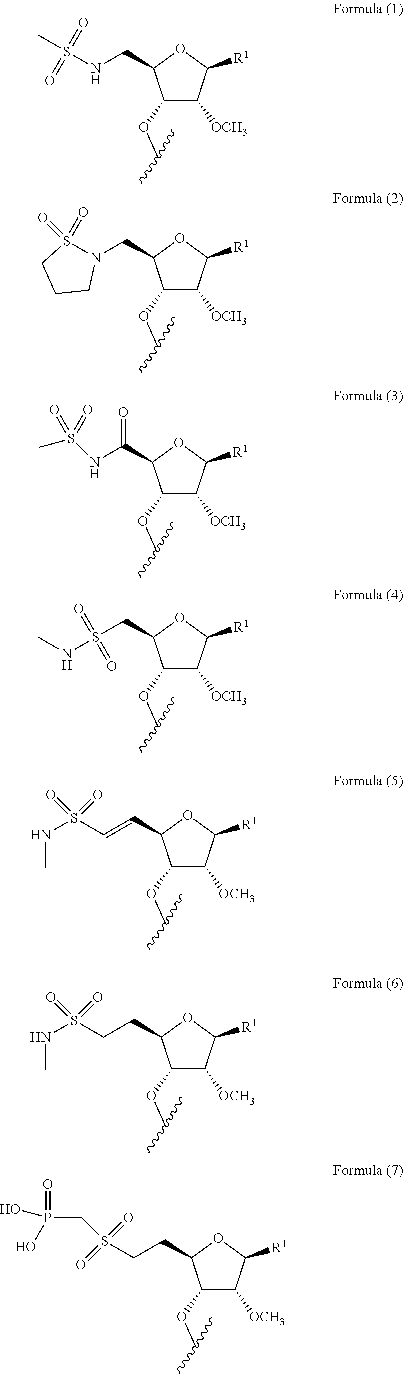

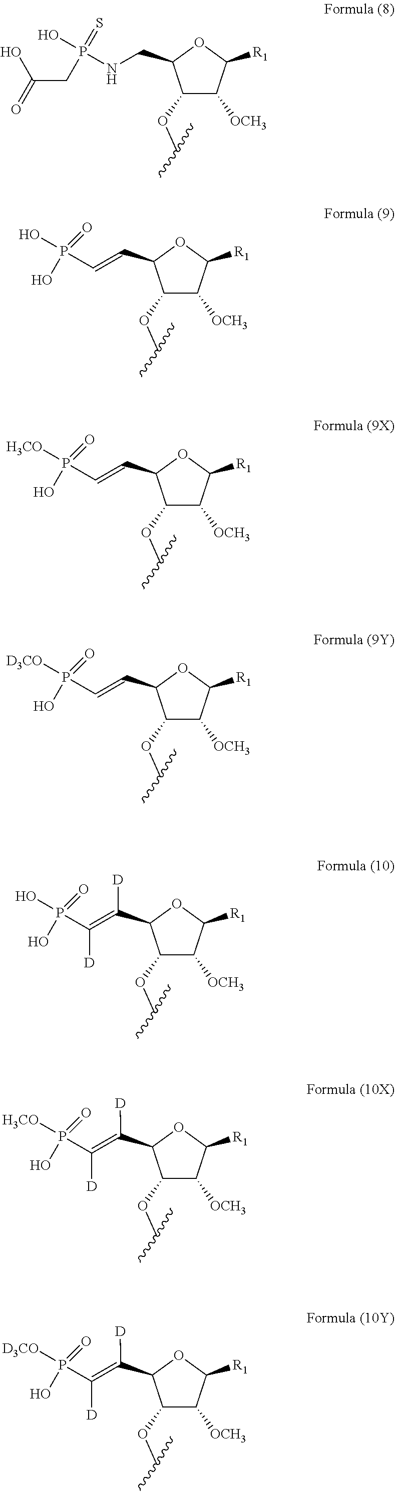

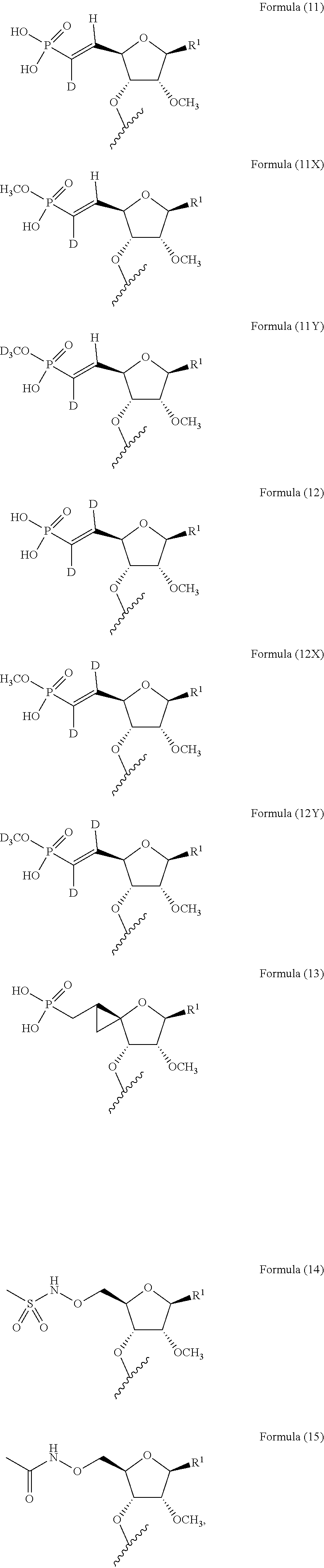

wherein R¹ is a nucleobase, aryl, heteroaryl, or H. In some embodiments, R¹ is an aryl. In some embodiments, the aryl is a phenyl.

In some embodiments, any of the siNA molecules disclosed herein comprise a 5′-stabilized end cap selected from the group consisting of Formulas (1A)-(15A), Formulas (9B)-(12B), Formulas (9AX)-(12AX), Formulas (9AY)-(12AY), Formulas (9BX)-(12BX), and Formulas (9BY)-(12BY):

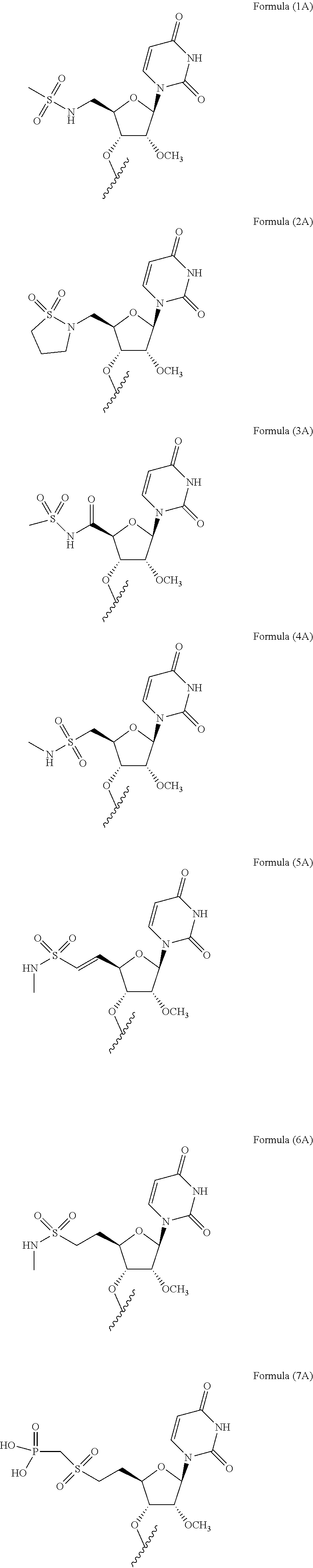

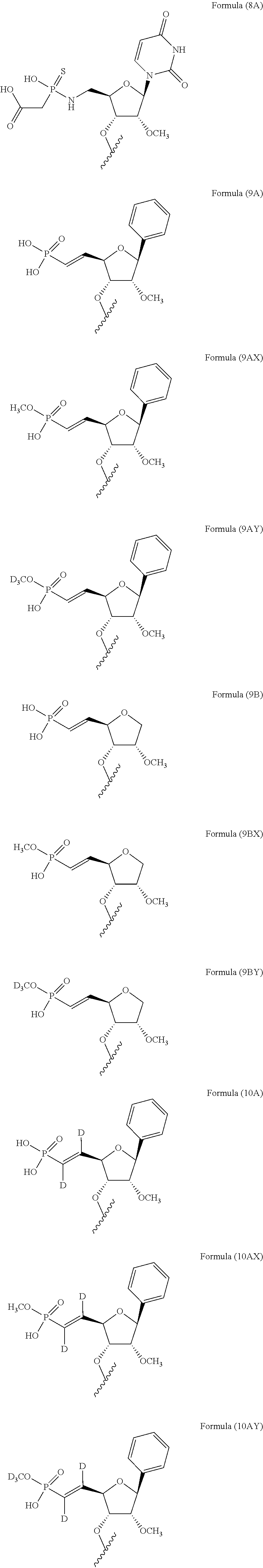

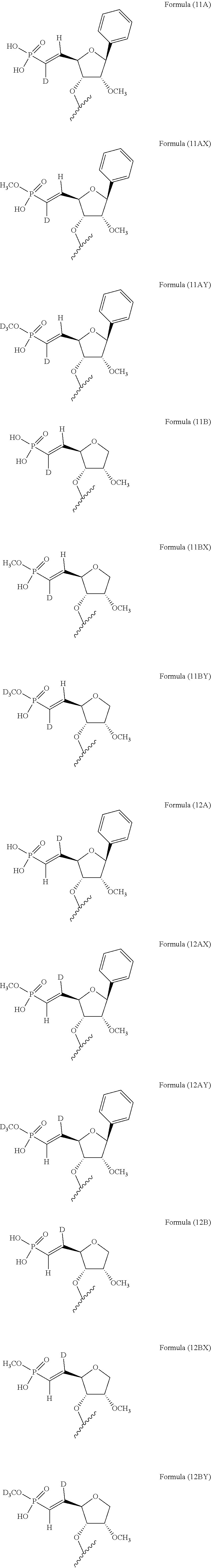

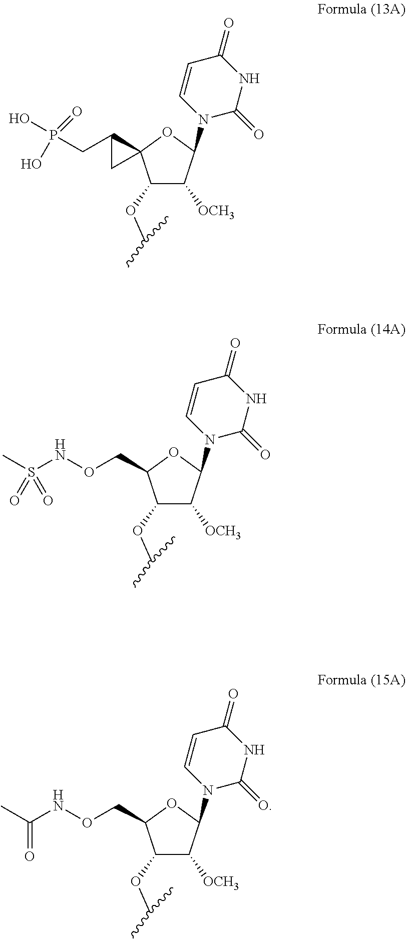

In some embodiments, any of the siNA molecules disclosed herein comprise a 5′-stabilized end cap selected from the group consisting of Formula (21) to Formula (35):

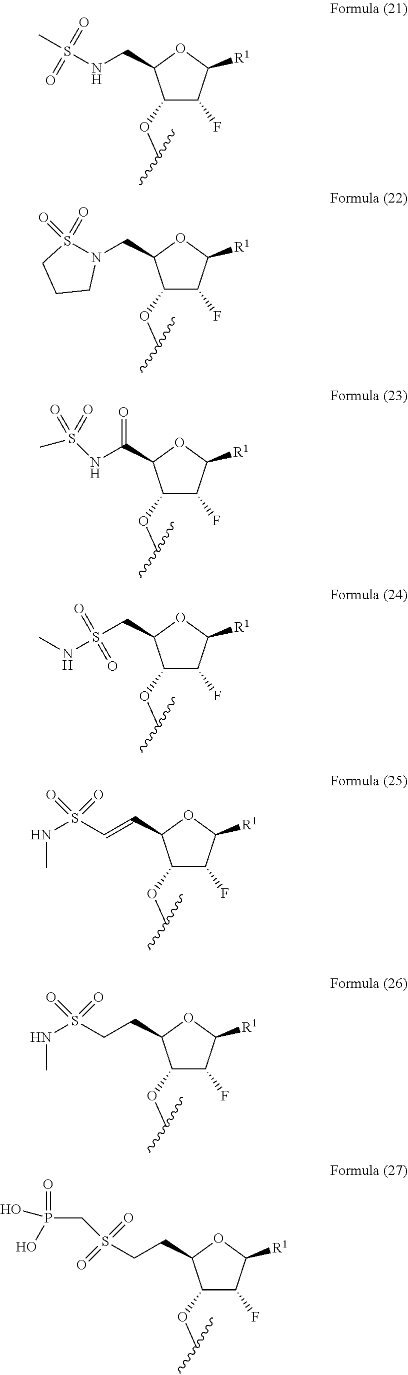

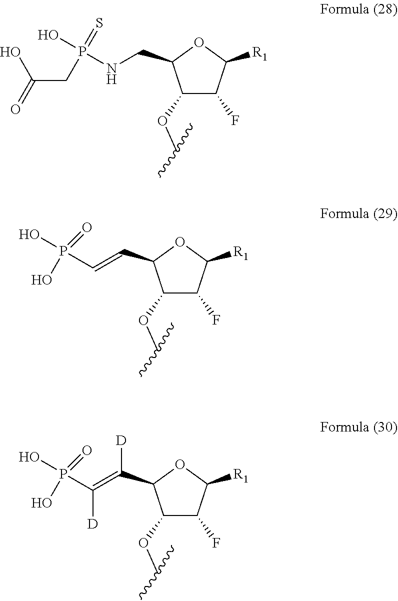

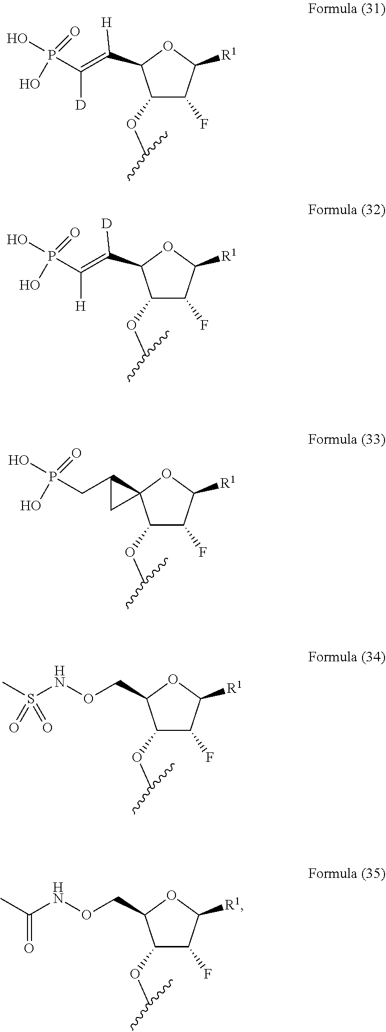

wherein R¹ is a nucleobase, aryl, heteroaryl, or H. In some embodiments, R¹ is an aryl. In some embodiments, the aryl is a phenyl.

In some embodiments, any of the siNA molecules disclosed herein comprise a 5′-stabilized end cap selected from the group consisting of Formulas (21A)-(35A), Formulas (29B)-(32B), Formulas (29AX)-(32AX), Formulas (29AY)-(32AY), Formulas (29BX)-(32BX), and Formulas (29BY)-(32BY):

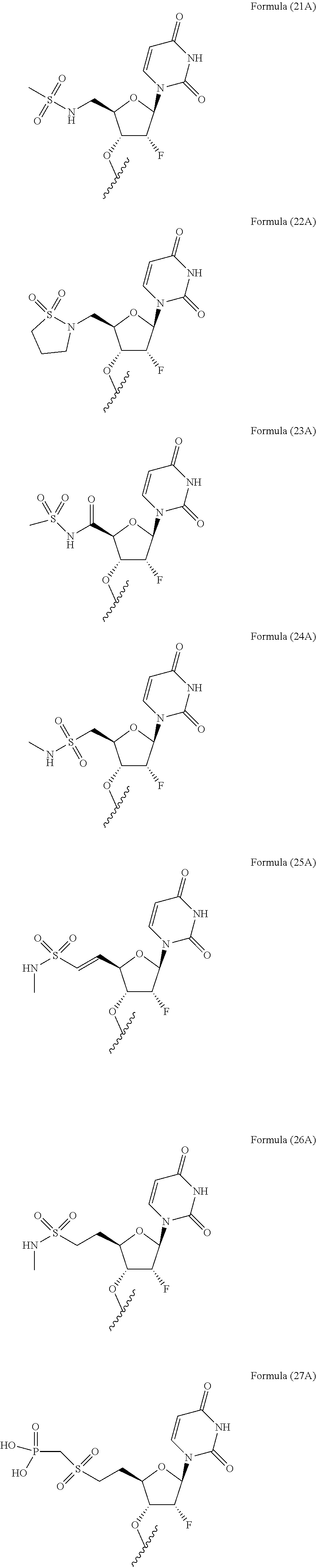

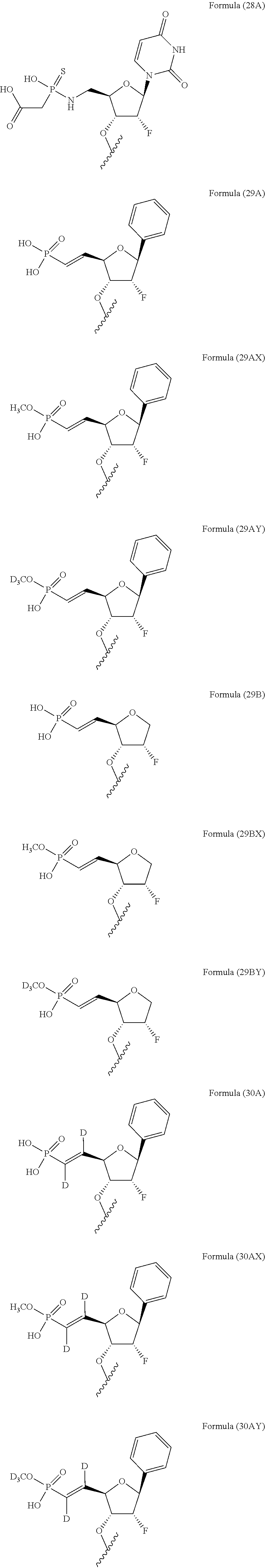

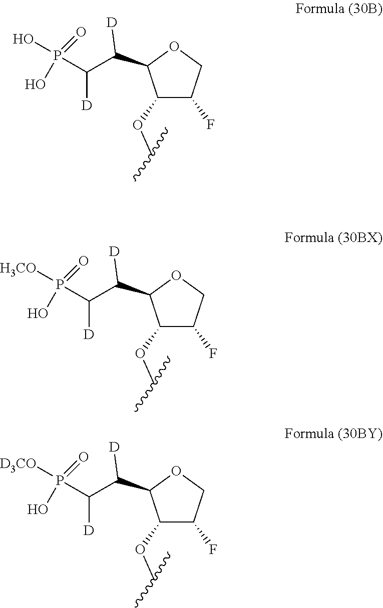

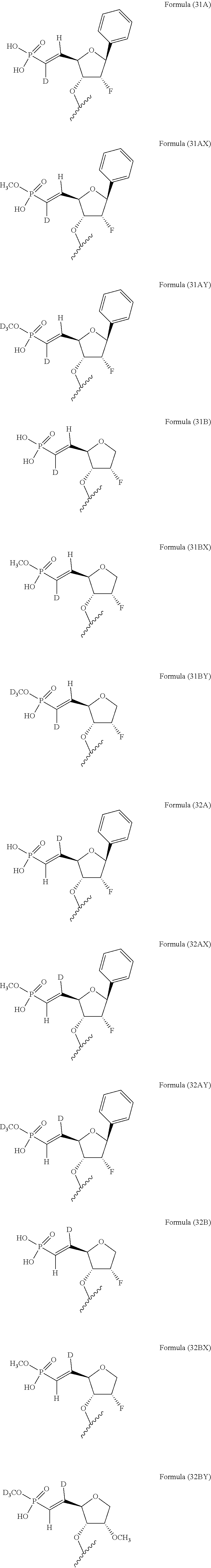

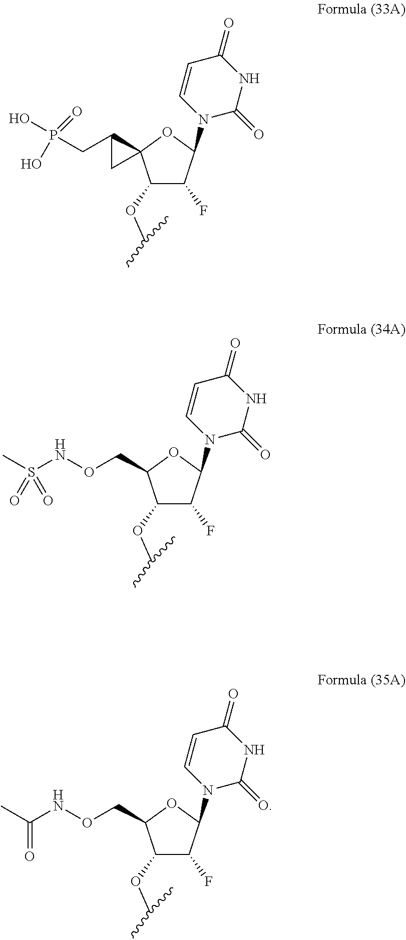

In some embodiments, the 5′-stabilized end cap is attached to the 5′ end of the antisense strand. In some embodiments, the 5′-stabilized end cap is attached to the 5′ end of the antisense strand via 1, 2, 3, 4, or 5 or more linkers. In some embodiments, one or more linkers are independently selected from the group consisting of a phosphodiester (p or po) linker, phosphorothioate (ps) linker, phosphoramidite (HEG) linker, triethylene glycol (TEG) linker, and/or phosphorodithioate linker.

### Linkers

In some embodiments, any of the siRNAs, sense strands, first nucleotide sequences, antisense strands, and/or second nucleotide sequences disclosed herein comprise 1, 2, 3, 4, 5, 6, 7, 8, 9, 10, 11, 12, 13, 14, 15, 16, 17, 18, 19, 20, 21, 22, 23 or more internucleoside linkers. In some embodiments, 1, 2, 3, 4, 5, 6, 7, 8, 9, 10 or more internucleoside linkers are independently selected from the group consisting of a phosphodiester (p or po) linker, phosphorothioate (ps) linker, or phosphorodithioate linker.

In some embodiments, any of the siRNAs, sense strands, first nucleotide sequences, antisense strands, and/or second nucleotide sequences disclosed herein further comprise 1, 2, 3, 4 or more linkers that attach a conjugated moiety, phosphorylation blocker, and/or 5′ end cap to the siRNA, sense strand, first nucleotide sequence, antisense strand, and/or second nucleotide sequences. In some embodiments, the 1, 2, 3, 4 or more linkers are independently selected from the group consisting of a phosphodiester (p or po) linker, phosphorothioate (ps) linker, phosphoramidite (HEG) linker, triethylene glycol (TEG) linker, and/or phosphorodithioate linker. In some embodiments, the one or more linkers are independently selected from the group consisting of p-(ps)₂, (ps)2-p-TEG-p, (ps)2-p-HEG-p, and (ps)2-p-(HEG-p)2.

### Specific Embodiments

The present disclosure provides numerous siNA that can be used to treat or prevent viral infections, specifically coronavirus (e.g., SARS-CoV-2) infections, such as COVID-19. Table 3, below, provides a non-limiting list of siNA that incorporate the nucleic acid sequences, modified nucleotides, phosphorylation blockers, 5′ stabilized end caps, and/or linkers of the foregoing sections. Those of skill in the art will understand that other exemplary siNA can be constructed by combining the sequences disclosed in Table 1 (or fragments of the sequences disclosed in Table 2) with the modified nucleotides, phosphorylation blockers, 5′ stabilized end caps, and/or linkers of the foregoing sections.

TABLE 3
Exemplary siNA

| | SEQ | | SEQ | |
|---|---|---|---|---|
| | ID | | ID | |
| Name | NO: | Sense Sequence (5′→3′) | NO: | Antisense Sequence (5′→3′) |
| ds- | 4383 | rGrCrUrCrCrUrArArUrUrArCrArCrUr | 4605 | rGrUrUrGrArGrUrGrUrArArUrUrArGr |
| siNA- | | CrArArCTT | | GrArGrCTT |
| 001 | | | | |
| ds- | 4384 | rGrGrArUrGrArGrGrArArGrGrCrArAr | 4606 | rUrArArArUrUrGrCrCrUrUrCrCrUrCr |
| siNA- | | UrUrUrATT | | ArUrCrCTT |
| 002 | | | | |
| ds- | 4385 | rCrCrGrCrUrGrGrArGrArGrCrArArCr | 4607 | rUrGrCrArGrUrUrGrCrUrCrUrCrCrUr |
| siNA- | | UrGrCrATT | | GrCrGrGTT |
| 003 | | | | |
| ds- | 4386 | rGrCrUrArUrGrArArArCrGrArUrArUr | 4608 | rGrCrCrCrArUrArUrCrGrUrUrUrCrAr |
| siNA- | | GrGrGrCTT | | UrArGrCTT |
| 004 | | | | |
| ds- | 4387 | fUpsmApsfGmCfAmGfCmAfUmUfA | 4609 | mCpsfApsmGfGmAfUmGfGmUfAmAf |
| siNA- | | mCfCmAfUmCfCmUfGpsTpsT | | UmGfCmUfGmCfUmATpsT |
| 005 | | | | |
| ds- | 4388 | mCpsfApsmAmCmAmCmGmGmAmC | 4610 | mUpsmApsfCmGmGmUfUfUfCmGmU |
| siNA- | | mGmAmAfAmCmCmGmUmApsTpsT | | fCmCmGmUmGfUmUmGTpsT |
| 006 | | | | |
| ds- | 4389 | mCpsmApsfCmGmUmCfCfAfAmCmU | 4611 | mGpsfCpsmAmAmAmCmUmGmAmG |
| siNA- | | fCmAmGmUmUfUmGmCTpsT | | mUmUmGfGmAmCmGmUmGpsTpsT |
| 007 | | | | |
| ds- | 4390 | mUpsmGpsfAmAmCmAfGfCfCmCm | 4612 | mGpsfApsmAmCmAmCmAmUmAmG |
| siNA- | | UfAmUmGmUmGfUmUmCTpsT | | mGmGmCfUmGmUmUmCmApsTpsT |
| 008 | | | | |
| ds- | 4391 | mApsmCpsfGmAmGmCfUfUfGmGm | 4613 | mGpsfGpsmAmUmCmAmGmUmGmC |
| siNA- | | CfAmCmUmGmAfUmCmCTpsT | | mCmAmAfGmCmUmCmGmUpsTpsT |
| 009 | | | | |
| ds- | 4392 | mUpsmCpsfAmUmGmUfGfGfUmAm | 4614 | mApsfApsmCmCmAmAmCmAmCmU |
| siNA- | | GfUmGmUmUmGfGmUmUTpsT | | mAmCmCfAmCmAmUmGmApsTpsT |
| 010 | | | | |
| ds- | 4393 | mApsmCpsfAmAmCmAfUfUfAmUm | 4615 | mGpsfCpsmAmUmUmGmUmUmGmA |
| siNA- | | CfAmAmCmAmAfUmGmCTpsT | | mUmAmAfUmGmUmUmGmUpsTpsT |
| 011 | | | | |
| ds- | 4394 | mApsfApsmAmCmCmUmAmCmAmA | 4616 | mGpsmGpsfAmAmCmCfAfCfCmUmU |
| siNA- | | mGmGmUfGmGmUmUmCmCpsTpsT | | fGmUmAmGmGfUmUmUTpsT |
| 012 | | | | |
| ds- | 4395 | mCpsmGpsfUmUmUmUfUfAfAmAm | 4617 | mGpsfCpsmAmAmAmCmCmCmGmU |
| siNA- | | CfGmGmGmUmUfUmGmCTpsT | | mUmUmAfAmAmAmAmCmGpsTpsT |
| 013 | | | | |
| ds- | 4396 | mGpsfUpsmGmCmCmGmCmAmCmG | 4618 | mGpsmUpsfCmUmUmAfCfAfCmCmG |
| siNA- | | mGmUmGfUmAmAmGmAmCpsTpsT | | fUmGmCmGmGfCmAmCTpsT |
| 014 | | | | |
| ds- | 4397 | TmUpsfGpsmAmCmGmUmGmAmUm | 4619 | mUpsmApsfCmCmAmCfAfUfAmUmA |
| siNA- | | AmUmAmUfGmUmGmGmUmApsTps | | fUmCmAmCmGfUmCmATpsT |
| 015 | | | | |
| ds- | 4398 | mGpsmGpsfAmUmGmUfAfAfAmCm | 4620 | mGpsfCpsmUmAmUmGmUmAmAmG |
| siNA- | | UfUmAmCmAmUfAmGmCTpsT | | mUmUmUfAmCmAmUmCmCpsTpsT |
| 016 | | | | |
| ds- | 4399 | mApsfCpsmCmGmGmGmUmUmUmG | 4621 | mUpsmCpsfAmAmAmCfUfGfUmCmA |
| siNA- | | mAmCmAfGmUmUmUmGmApsTpsT | | fAmAmCmCmCfGmGmUTpsT |
| 017 | | | | |
| ds- | 4400 | mUpsmGpsfUmCmAmAfAfCfCmCm | 4622 | mApsfApsmAmAmUmUmAmCmCmG |
| siNA- | | GfGmUmAmAmUfUmUmUTpsT | | mGmGmUfUmUmGmAmCmApsTpsT |
| 018 | | | | |
| ds- | 4401 | mUpsmApsfAmGmUmAfUfGfCmCm | 4623 | mUpsfGpsmCmAmCmUmAmAmUmG |
| siNA- | | AfUmUmAmGmUfGmCmATpsT | | mGmCmAfUmAmCmUmUmApsTpsT |
| 019 | | | | |
| ds- | 4402 | mUpsmGpsfCmCmAmUfUfAfGmUm | 4624 | mApsfUpsmUmCmUmUmUmGmCmA |
| siNA- | | GfCmAmAmAmGfAmAmUTpsT | | mCmUmAfAmUmGmGmCmApsTpsT |
| 020 | | | | |
| ds- | 4403 | mGpsfCpsmGmAmGmCmUmCmUmA | 4625 | mUpsmGpsfCmAmAmAfGfAfAmUmA |
| siNA- | | mUmUmCfUmUmUmGmCmApsTpsT | | fGmAmGmCmUfCmGmCTpsT |
| 021 | | | | |
| ds- | 4404 | mGpsfApsmCmAmCmCmAmGmCmU | 4626 | mUpsmCpsfGmCmAmCfCfGfUmAmG |
| siNA- | | mAmCmGfGmUmGmCmGmApsTpsT | | fCmUmGmGmUfGmUmCTpsT |
| 022 | | | | |
| ds- | 4405 | mCpsmApsfAmUmAmGfCfCfGmCmC | 4627 | mCpsfCpsmUmCmUmAmGmUmGmG |
| siNA- | | fAmCmUmAmGfAmGmGTpsT | | mCmGmGfCmUmAmUmUmGpsTpsT |
| 023 | | | | |
| ds- | 4406 | mApsmCpsfUmGmCmUfUfAfUmGm | 4628 | mCpsfApsmCmUmAmUmUmAmGmC |
| siNA- | | CfUmAmAmUmAfGmUmGTpsT | | mAmUmAfAmGmCmAmGmUpsTpsT |
| 024 | | | | |
| ds- | 4407 | mApsfCpsmAmUmCmAmGmCmAmU | 4629 | mApsmUpsfCmAmGmGfAfGfUmAmU |
| siNA- | | mAmCmUfCmCmUmGmAmUpsTpsT | | fGmCmUmGmAfUmGmUTpsT |
| 025 | | | | |
| ds- | 4408 | mUpsmApsfGmGmAmGfGfUfAmUm | 4630 | mUpsfApsmAmUmAmGmCmUmCmA |
| siNA- | | GfAmGmCmUmAfUmUmATpsT | | mUmAmCfCmUmCmCmUmApsTpsT |
| 026 | | | | |
| ds- | 4409 | mApsmCpsfUmAmUmGfGfUfGmAm | 4631 | mApsfCpsmAmAmCmAmGmCmAmU |
| siNA- | | UfGmCmUmGmUfUmGmUTpsT | | mCmAmCfCmAmUmAmGmUpsTpsT |
| 027 | | | | |
| ds- | 4410 | mApsfCpsmAmUmAmGmUmGmCmU | 4632 | mUpsmGpsfCmCmAmCfAfAfGmAmG |
| siNA- | | mCmUmUfGmUmGmGmCmApsTpsT | | fCmAmCmUmAfUmGmUTpsT |
| 028 | | | | |
| ds- | 4411 | mUpsfApsmUmAmCmAmCmUmAmU | 4633 | mUpsmCpsfUmGmCmUfCfGfCmAmU |
| siNA- | | mGmCmGfAmGmCmAmGmApsTpsT | | fAmGmUmGmUfAmUmATpsT |
| 029 | | | | |
| ds- | 4412 | mApsmApsfUmUmCmAfAfAfGmUm | 4634 | mGpsfUpsmUmGmAmAmUmUmCmA |
| siNA- | | GfAmAmUmUmCfAmAmCTpsT | | mCmUmUfUmGmAmAmUmUpsTpsT |
| 030 | | | | |
| ds- | 4413 | mApsfGpsmGmAmAmCmAmUmGm | 4635 | mUpsmApsfGmGmUmCfCfAfGmAmC |
| siNA- | | UmCmUmGfGmAmCmCmUmApsTps | | fAmUmGmUmUfCmCmUTpsT |
| 031 | | T | | |
| ds- | 4414 | mUpsfGpsmAmAmUmAmUmGmAm | 4636 | mUpsmApsfUmGmAmCfUfAfUmGmU |
| siNA- | | CmAmUmAfGmUmCmAmUmApsTps | | fCmAmUmAmUfUmCmATpsT |
| 032 | | T | | |
| ds- | 4415 | mUpsmUpsfUmGmAmGfCfUfUmUm | 4637 | mGpsfCpsmUmUmAmGmCmCmCmA |
| siNA- | | GfGmGmCmUmAfAmGmCTpsT | | mAmAmGfCmUmCmAmAmApsTpsT |
| 033 | | | | |
| ds- | 4416 | mUpsmApsfAmUmGmAfUfGfAmAm | 4638 | mUpsfUpsmUmGmCmGmAmCmAmU |
| siNA- | | UfGmUmCmGmCfAmAmATpsT | | mUmCmAfUmCmAmUmUmApsTpsT |
| 034 | | | | |
| ds- | 4417 | mGpsmApsfGmUmAmCfGfAfAmCm | 4639 | mApsfGpsmUmAmCmAmUmAmAmG |
| siNA- | | UfUmAmUmGmUfAmCmUTpsT | | mUmUmCfGmUmAmCmUmCpsTpsT |
| 035 | | | | |
| ds- | 4418 | mGpsmGpsfUmAmCmGfUfUfAmAm | 4640 | mUpsfApsmUmUmAmAmCmUmAmU |
| siNA- | | UfAmGmUmUmAfAmUmATpsT | | mUmAmAfCmGmUmAmCmCpsTpsT |
| 036 | | | | |
| ds- | 4419 | mGpsfApsmAmAmAmAmGmAmAm | 4641 | mApsmApsfUmAmGmCfGfUfAmCmU |
| siNA- | | GmUmAmCfGmCmUmAmUmUpsTps | | fUmCmUmUmUfUmUmCTpsT |
| 037 | | T | | |
| ds- | 4420 | mGpsfCpsmAmAmGmAmAmUmAmC | 4642 | mGpsmCpsfUmUmUmCfGfUfGmGmU |
| siNA- | | mCmAmCfGmAmAmAmGmCpsTpsT | | fAmUmUmCmUfUmGmCTpsT |
| 038 | | | | |
| ds- | 4421 | mApsfCpsmAmAmUmCmGmAmAmG | 4643 | mCpsmUpsfUmAmCmUfGfCfGmCmU |
| siNA- | | mCmGmCfAmGmUmAmAmGpsTpsT | | fUmCmGmAmUfUmGmUTpsT |
| 039 | | | | |
| ds- | 4422 | mUpsmUpsfCmUmGmGfUfCfUmAm | 4644 | mUpsfApsmGmUmUmCmGmUmUmU |
| siNA- | | AfAmCmGmAmAfCmUmATpsT | | mAmGmAfCmCmAmGmAmApsTpsT |
| 040 | | | | |
| ds- | 4423 | mUpsfApsmAmUmAmAmGmAmAm | 4645 | mCpsmApsfCmGmAmAfCfGfCmUmU |
| siNA- | | AmGmCmGfUmUmCmGmUmGpsTps | | fUmCmUmUmAfUmUmATpsT |
| 041 | | T | | |
| ds- | 4424 | mUpsfGpsmUmAmUmGmCmAmGmC | 4646 | mUpsmCpsfAmGmGmUfUfUfUmGmC |
| siNA- | | mAmAmAfAmCmCmUmGmApsTpsT | | fUmGmCmAmUfAmCmATpsT |
| 042 | | | | |
| ds- | 4425 | mCpsfApsmUmCmUmGmUmUmGmU | 4647 | mCpsmApsfGmUmAmAfGfUfGmAmC |
| siNA- | | mCmAmCfUmUmAmCmUmGpsTpsT | | fAmAmCmAmGfAmUmGTpsT |
| 043 | | | | |
| ds- | 4426 | mUpsmApsfCmCmCmAfAfUfAmAm | 4648 | mGpsfApsmCmGmCmAmGmUmAmU |
| siNA- | | UfAmCmUmGmCfGmUmCTpsT | | mUmAmUfUmGmGmGmUmApsTpsT |
| 044 | | | | |
| ds- | 4427 | mCpsfUpsmUmCmGmGmUmAmGmU | 4649 | mApsmApsfAmUmUmGfGfCfUmAmC |
| siNA- | | mAmGmCfCmAmAmUmUmUpsTpsT | | fUmAmCmCmGfAmAmGTpsT |
| 045 | | | | |
| ds- | 4428 | mCpsmApsfAmAmAmGfGfCfUmUm | 4650 | mUpsfCpsmUmGmCmGmUmAmGmA |
| siNA- | | CfUmAmCmGmCfAmGmATpsT | | mAmGmCfCmUmUmUmUmGpsTpsT |
| 046 | | | | |
| ds- | 4429 | mUpsmGpsfUmCmAmCfUfAfAmGm | 4651 | mApsfGpsmCmAmGmAmUmUmUmC |
| siNA- | | AfAmAmUmCmUfGmCmUTpsT | | mUmUmAfGmUmGmAmCmApsTpsT |
| 047 | | | | |
| ds- | 4430 | mGpsmApsfCmAmAmGfGfAfAmCm | 4652 | mUpsfUpsmGmUmAmAmUmCmAmG |
| siNA- | | UfGmAmUmUmAfCmAmATpsT | | mUmUmCfCmUmUmGmUmCpsTpsT |
| 048 | | | | |
| ds- | 4431 | mCpsmApsfUmGmGmAfAfGfUmCm | 4653 | mCpsfGpsmAmAmGmGmUmGmUmG |
| siNA- | | AfCmAmCmCmUfUmCmGTpsT | | mAmCmUfUmCmCmAmUmGpsTpsT |
| 049 | | | | |
| ds- | 4432 | mCpsfApsmUmUmCmUmGmCmAmC | 4654 | mUpsmCpsfUmAmCmUfCfUfUmGmU |
| siNA- | | mAmAmGfAmGmUmAmGmApsTpsT | | fGmCmAmGmAfAmUmGTpsT |
| 050 | | | | |
| ds- | 4433 | mCpsmApsfCmAmUmAfGfCfAmAm | 4655 | mGpsfApsmUmUmAmAmAmGmAmU |
| siNA- | | UfCmUmUmUmAfAmUmCTpsT | | mUmGmCfUmAmUmGmUmGpsTpsT |
| 051 | | | | |
| ds- | 4434 | mApsfApsmAmUmGmUmGmGmUm | 4656 | mUpsmGpsfAmAmAmGfAfGfCmCmA |
| siNA- | | GmGmCmUfCmUmUmUmCmApsTps | | fCmCmAmCmAfUmUmUTpsT |
| 052 | | T | | |
| ds- | 4435 | mUpsfUpsmUmAmCmAmCmAmUmU | 4657 | mApsmApsfGmAmGmCfCfCfUmAmA |
| siNA- | | mAmGmGfGmCmUmCmUmUpsTpsT | | fUmGmUmGmUfAmAmATpsT |
| 053 | | | | |
| ds- | 4436 | mUpsmApsmCmGfGmUmUmUfCfGf | 4658 | mUpsfGpsmCmAmAmCmAmCmGmG |
| siNA- | | UmCmCfGmUmGmUmUfGmCpsmA | | mAmCmGfAmAmAmCmCmGmUmAp |
| 054 | | | | sTpsT |
| ds- | 4437 | mApsfApsmCmUmGmAmGmUmUm | 4659 | mApsmApsmAmCfAmCmAmCfGfUfC |
| siNA- | | GmGmAmCfGmUmGmUmGmUmUm | | mCmAfAmCmUmCmAfGmUpsmU |
| 055 | | UpsTpsT | | |
| ds- | 4438 | mUpsmGpsmAmAfCmAmGmCfCfCf | 4660 | mApsfUpsmGmAmAmCmAmCmAmU |
| siNA- | | UmAmUfGmUmGmUmUfCmApsmU | | mAmGmGfGmCmUmGmUmUmCmAp |
| 056 | | | | sTpsT |
| ds- | 4439 | mUpsmApsmUmUfUmAmAmAfAfCf | 4661 | mApsfUpsmUmGmUmCmAmGmUmA |
| siNA- | | UmUmAfCmUmGmAmCfAmApsmU | | mAmGmUfUmUmUmAmAmAmUmA |
| 057 | | | | psTpsT |
| ds- | 4440 | mUpsfUpsmUmUmCmCmAmCmUmA | 4662 | mCpsmUpsmCmUfGmAmAmGfAfAfG |
| siNA- | | mCmUmUfCmUmUmCmAmGmAmG | | mUmAfGmUmGmGmAfAmApsmA |
| 058 | | psTpsT | | |
| ds- | 4441 | mUpsfGpsmUmUmAmAmAmAmCmC | 4663 | mGpsmUpsmGmGfUmAmGmUfGfUf |
| siNA- | | mAmAmCfAmCmUmAmCmCmAmC | | UmGmGfUmUmUmUmAfAmCpsmA |
| 059 | | psTpsT | | |
| ds- | 4442 | mGpsfUpsmAmAmCmAmAmAmCmC | 4664 | mApsmCpsmCmAfCmCmUmUfGfUfA |
| siNA- | | mUmAmCfAmAmGmGmUmGmGmU | | mGmGfUmUmUmGmUfUmApsmC |
| 060 | | psTpsT | | |
| ds- | 4443 | mUpsmGpsmUmUfGmUmGmUfAfCf | 4665 | mUpsfApsmCmCmAmGmUmGmUmG |
| siNA- | | AmCmAfCmAmCmUmGfGmUpsmA | | mUmGmUfAmCmAmCmAmAmCmAp |
| 061 | | | | sTpsT |
| ds- | 4444 | mApsmApsmAmGfGmUmUmAfUfGf | 4666 | mApsfCpsmAmAmCmUmAmCmAmG |
| siNA- | | GmCmUfGmUmAmGmUfUmGpsmU | | mCmCmAfUmAmAmCmCmUmUmUp |
| 062 | | | | sTpsT |
| ds- | 4445 | mUpsfUpsmAmCmAmCmCmGmCmA | 4667 | mUpsmUpsmAmAfAmCmGmGfGfUfU |
| siNA- | | mAmAmCfCmCmGmUmUmUmAmA | | mUmGfCmGmGmUmGfUmApsmA |
| 063 | | psTpsT | | |
| ds- | 4446 | mGpsmUpsmGmUfAmAmGmUfGfCf | 4668 | mUpsfApsmAmGmAmCmGmGmGmC |
| siNA- | | AmGmCfCmCmGmUmCfUmUpsmA | | mUmGmCfAmCmUmUmAmCmAmCp |
| 064 | | | | sTpsT |
| ds- | 4447 | mApsmCpsmAmUfGmGmUmAfCfCf | 4669 | mGpsfUpsmGmAmUmAmUmAmUmG |
| siNA- | | AmCmAfUmAmUmAmUfCmApsmC | | mUmGmGfUmAmCmCmAmUmGmUp |
| 065 | | | | sTpsT |
| ds- | 4448 | mUpsfUpsmAmCmCmGmGmGmUmU | 4670 | mUpsmCpsmAmAfAmCmUmGfUfCfA |
| siNA- | | mUmGmAfCmAmGmUmUmUmGmA | | mAmAfCmCmCmGmGfUmApsmA |
| 066 | | psTpsT | | |
| ds- | 4449 | mUpsfGpsmAmGmCmAmAmAmGm | 4671 | mApsmApsmAmAfCmAmCmUfUfCfU |
| siNA- | | AmAmGmAfAmGmUmGmUmUmUm | | mUmCfUmUmUmGmCfUmCpsmA |
| 067 | | UpsTpsT | | |
| ds- | 4450 | mApsfGpsmCmCmAmCmCmAmUmC | 4672 | mUpsmUpsmGmAfUmUmGmUfUfAfC |
| siNA- | | mGmUmAfAmCmAmAmUmCmAmA | | mGmAfUmGmGmUmGfGmCpsmU |
| 068 | | psTpsT | | |
| ds- | 4451 | mApsmApsmAmUfGmAmAmUfCfUf | 4673 | mUpsfGpsmGmCmAmUmAmCmUmU |
| siNA- | | UmAmAfGmUmAmUmGfCmCpsmA | | mAmAmGfAmUmUmCmAmUmUmU |
| 069 | | | | psTpsT |
| ds- | 4452 | mApsfUpsmUmCmUmUmUmGmCmA | 4674 | mUpsmApsmUmGfCmCmAmUfUfAfG |
| siNA- | | mCmUmAfAmUmGmGmCmAmUmA | | mUmGfCmAmAmAmGfAmApsmU |
| 070 | | psTpsT | | |
| ds- | 4453 | mCpsfUpsmAmUmUmCmUmUmUmG | 4675 | mUpsmGpsmCmCfAmUmUmAfGfUfG |
| siNA- | | mCmAmCfUmAmAmUmGmGmCmA | | mCmAfAmAmGmAmAfUmApsmG |
| 071 | | psTpsT | | |
| ds- | 4454 | mCpsfUpsmAmCmGmGmUmGmCmG | 4676 | mApsmGpsmAmAfUmAmGmAfGfCfU |
| siNA- | | mAmGmCfUmCmUmAmUmUmCmU | | mCmGfCmAmCmCmGfUmApsmG |
| 072 | | psTpsT | | |
| ds- | 4455 | mApsmUpsmAmGfAmGmCmUfCfGf | 4677 | mCpsfApsmGmCmUmAmCmGmGmU |
| siNA- | | CmAmCfCmGmUmAmGfCmUpsmG | | mGmCmGfAmGmCmUmCmUmAmUp |
| 073 | | | | sTpsT |
| ds- | 4456 | mUpsmCpsmGmCfAmCmCmGfUfAf | 4678 | mGpsfApsmGmAmCmAmCmCmAmG |
| siNA- | | GmCmUfGmGmUmGmUfCmUpsmC | | mCmUmAfCmGmGmUmGmCmGmAp |
| 074 | | | | sTpsT |
| ds- | 4457 | mApsmApsmCmUfGmCmUmUfAfUf | 4679 | mApsfCpsmAmCmUmAmUmUmAmG |
| siNA- | | GmCmUfAmAmUmAmGfUmGpsmU | | mCmAmUfAmAmGmCmAmGmUmUp |
| 075 | | | | sTpsT |
| ds- | 4458 | mUpsfUpsmAmGmUmAmAmGmGm | 4680 | mGpsmApsmCmUfGmAmGmAfCfUfG |
| siNA- | | UmCmAmGfUmCmUmCmAmGmUm | | mAmCfCmUmUmAmCfUmApsmA |
| 076 | | CpsTpsT | | |
| ds- | 4459 | mApsfCpsmCmUmUmUmUmUmCmA | 4681 | mGpsmApsmGmUfAmCmAmCfCfUfU |
| siNA- | | mAmAmGfGmUmGmUmAmCmUmC | | mUmGfAmAmAmAmAfGmGpsmU |
| 077 | | psTpsT | | |
| ds- | 4460 | mUpsmGpsmGmUfAmCmUmGfGfUf | 4682 | mApsfApsmAmUmGmAmCmUmCmU |
| siNA- | | AmAmGfAmGmUmCmAfUmUpsmU | | mUmAmCfCmAmGmUmAmCmCmAp |
| 078 | | | | sTpsT |
| ds- | 4461 | mUpsmCpsmUmGfCmUmAmAfUfCf | 4683 | mApsfGpsmUmAmGmCmAmGmCmA |
| siNA- | | UmUmGfCmUmGmCmUfAmCpsmU | | mAmGmAfUmUmAmGmCmAmGmA |
| 079 | | | | psTpsT |
| ds- | 4462 | mCpsfGpsmCmUmAmUmUmAmAmC | 4684 | mGpsmUpsmAmCfGmUmUmAfAfUfA |
| siNA- | | mUmAmUfUmAmAmCmGmUmAmC | | mGmUfUmAmAmUmAfGmCpsmG |
| 080 | | psTpsT | | |
| ds- | 4463 | mUpsmUpsmCmUfUmGmCmUfUfUf | 4685 | mApsfGpsmAmAmUmAmCmCmAmC |
| siNA- | | CmGmUfGmGmUmAmUfUmCpsmU | | mGmAmAfAmGmCmAmAmGmAmA |
| 081 | | | | psTpsT |
| ds- | 4464 | mCpsmUpsmUmAfCmUmGmCfGfCf | 4686 | mApsfCpsmAmCmAmAmUmCmGmA |
| siNA- | | UmUmCfGmAmUmUmGfUmGpsmU | | mAmGmCfGmCmAmGmUmAmAmGp |
| 082 | | | | sTpsT |
| ds- | 4465 | mCpsmGpsmCmUfUmCmGmAfUfUf | 4687 | mApsfGpsmUmAmCmGmCmAmCmA |
| siNA- | | GmUmGfUmGmCmGmUfAmCpsmU | | mCmAmAfUmCmGmAmAmGmCmGp |
| 083 | | | | sTpsT |
| ds- | 4466 | mUpsfApsmAmCmAmAmUmAmUm | 4688 | mCpsmGpsmUmAfCmUmGmCfUfGfC |
| siNA- | | UmGmCmAfGmCmAmGmUmAmCm | | mAmAfUmAmUmUmGfUmUpsmA |
| 084 | | GpsTpsT | | |
| ds- | 4467 | mUpsfCpsmGmUmUmUmAmGmAmC | 4689 | mCpsmUpsmGmAfUmCmUmUfCfUfG |
| siNA- | | mCmAmGfAmAmGmAmUmCmAmG | | mGmUfCmUmAmAmAfCmGpsmA |
| 085 | | psTpsT | | |
| ds- | 4468 | mUpsmCpsmAmCfGmAmAmCfGfCf | 4690 | mGpsfUpsmAmAmUmAmAmGmAmA |
| siNA- | | UmUmUfCmUmUmAmUfUmApsmC | | mAmGmCfGmUmUmCmGmUmGmAp |
| 086 | | | | sTpsT |
| ds- | 4469 | mUpsmApsmAmAfCmGmAmAfCfAf | 4691 | mApsfApsmUmAmAmUmUmUmUmC |
| siNA- | | UmGmAfAmAmAmUmUfAmUpsmU | | mAmUmGfUmUmCmGmUmUmUmA |
| 087 | | | | psTpsT |
| ds- | 4470 | mApsmApsmUmUfGmAmCmUfUfCf | 4692 | mApsfApsmGmCmAmCmAmAmAmU |
| siNA- | | UmAmUfUmUmGmUmGfCmUpsmU | | mAmGmAfAmGmUmCmAmAmUmU |
| 088 | | | | psTpsT |
| ds- | 4471 | mCpsmApsmAmUfAmAmUmAfCfUf | 4693 | mApsfApsmCmCmAmAmGmAmCmG |
| siNA- | | GmCmGfUmCmUmUmGfGmUpsmU | | mCmAmGfUmAmUmUmAmUmUmG |
| 089 | | | | psTpsT |
| ds- | 4472 | mApsmUpsmUmGfGmCmUmAfCfUf | 4694 | mApsfGpsmCmUmCmUmUmCmGmG |
| siNA- | | AmCmCfGmAmAmGmAfGmCpsmU | | mUmAmGfUmAmGmCmCmAmAmUp |
| 090 | | | | sTpsT |
| ds- | 4473 | mCpsfCpsmUmUmGmAmGmGmAmA | 4695 | mCpsmGpsmUmGfCmUmAmCfAfAfC |
| siNA- | | mGmUmUfGmUmAmGmCmAmCmG | | mUmUfCmCmUmCmAfAmGpsmG |
| 091 | | psTpsT | | |
| ds- | 4474 | mUpsfGpsmCmUmCmUmCmAmAmG | 4696 | mGpsmApsmUmUfGmAmAmCfCfAfG |
| siNA- | | mCmUmGfGmUmUmCmAmAmUmC | | mCmUfUmGmAmGmAfGmCpsmA |
| 092 | | psTpsT | | |
| ds- | 4475 | mUpsmGpsmGmUfAmAmAmGfGfCf | 4697 | mUpsfGpsmUmUmGmUmUmGmUmU |
| siNA- | | CmAmAfCmAmAmCmAfAmCpsmA | | mGmGmCfCmUmUmUmAmCmCmAp |
| 093 | | | | sTpsT |
| ds- | 4476 | mGpsfCpsmCmUmCmAmGmCmAmG | 4698 | mUpsmApsmAmGfAmAmAmUfCfUfG |
| siNA- | | mCmAmGfAmUmUmUmCmUmUmA | | mCmUfGmCmUmGmAfGmGpsmC |
| 094 | | psTpsT | | |
| ds- | 4477 | mUpsfUpsmUmGmUmAmAmUmCm | 4699 | mApsmGpsmAmCfAmAmGmGfAfAfC |
| siNA- | | AmGmUmUfCmCmUmUmGmUmCm | | mUmGfAmUmUmAmCfAmApsmA |
| 095 | | UpsTpsT | | |
| ds- | 4478 | mApsmApsmAmUfUmGmGmAfUfGf | 4700 | mUpsfGpsmGmAmUmCmUmUmUmG |
| siNA- | | AmCmAfAmAmGmAmUfCmCpsmA | | mUmCmAfUmCmCmAmAmUmUmUp |
| 096 | | | | sTpsT |
| ds- | 4479 | psTpsTmCpsfCpsmUmUmUmUmUmAmGmG | 4701 | mCpsmApsmCmCfAmAmCmAfGfAfG |
| siNA- | | mCmUmCfUmGmUmUmGmGmUmG | | mCmCfUmAmAmAmAfAmGpsmG |
| 097 | | | | |
| ds- | 4480 | mCpsmUpsmAmCfUmCmUmUfGfUf | 4702 | mApsfUpsmUmCmAmUmUmCmUmG |
| siNA- | | GmCmAfGmAmAmUmGfAmApsmU | | mCmAmCfAmAmGmAmGmUmAmGp |
| 098 | | | | sTpsT |
| ds- | 4481 | mUpsfCpsmGmAmUmCmGmUmAmC | 4703 | mGpsmGpsmCmCfAmCmGmCfGfGfA |
| siNA- | | mUmCmCfGmCmGmUmGmGmCmC | | mGmUfAmCmGmAmUfCmGpsmA |
| 099 | | psTpsT | | |
| ds- | 4482 | mUpsfUpsmUmUmAmCmAmCmAmU | 4704 | mGpsmApsmAmGfAmGmCmCfCfUfA |
| siNA- | | mUmAmGfGmGmCmUmCmUmUmC | | mAmUfGmUmGmUmAfAmApsmA |
| 100 | | psTpsT | | |
| ds- | 4483 | fUpsmApsfCmGfGmUfUmUfCmGfU | 4705 | mCpsfApsmAfCmAfCmGfGmAfCmGf |
| siNA- | | mCfCmGfUmGfUmUfGTpsT | | AmAfAmCfCmGfUmApsTpsT |
| 101 | | | | |
| ds- | 4484 | mGpsfCpsmAfAmAfCmUfGmAfGmU | 4706 | fCpsmApsfCmGfUmCfCmAfAmCfUm |
| siNA- | | fUmGfGmAfCmGfUmGpsTpsT | | CfAmGfUmUfUmGfCTpsT |
| 102 | | | | |
| ds- | 4485 | fUpsmGpsfAmAfCmAfGmCfCmCfUm | 4707 | mGpsfApsmAfCmAfCmAfUmAfGmGf |
| siNA- | | AfUmGfUmGfUmUfCTpsT | | GmCfUmGfUmUfCmApsTpsT |
| 103 | | | | |
| ds- | 4486 | fApsmCpsfGmAfGmCfUmUfGmGfCm | 4708 | mGpsfGpsmAfUmCfAmGfUmGfCmCf |
| siNA- | | AfCmUfGmAfUmCfCTpsT | | AmAfGmCfUmCfGmUpsTpsT |
| 104 | | | | |
| ds- | 4487 | fUpsmCpsfAmUfGmUfGmGfUmAfG | 4709 | mApsfApsmCfCmAfAmCfAmCfUmAf |
| siNA- | | mUfGmUfUmGfGmUfUTpsT | | CmCfAmCfAmUfGmApsTpsT |
| 105 | | | | |
| ds- | 4488 | fApsmCpsfAmAfCmAfUmUfAmUfCm | 4710 | mGpsfCpsmAfUmUfGmUfUmGfAmUf |
| siNA- | | AfAmCfAmAfUmGfCTpsT | | AmAfUmGfUmUfGmUpsTpsT |
| 106 | | | | |
| ds- | 4489 | mApsfApsmAfCmCfUmAfCmAfAmGf | 4711 | fGpsmGpsfAmAfCmCfAmCfCmUfUm |
| siNA- | | GmUfGmGfUmUfCmCpsTpsT | | GfUmAfGmGfUmUfUTpsT |
| 107 | | | | |
| ds- | 4490 | fCpsmGpsfUmUfUmUfUmAfAmAfC | 4712 | mGpsfCpsmAfAmAfCmCfCmGfUmUf |
| siNA- | | mGfGmGfUmUfUmGfCTpsT | | UmAfAmAfAmAfCmGpsTpsT |
| 108 | | | | |
| ds- | 4491 | mGpsfUpsmGfCmCfGmCfAmCfGmGf | 4713 | fGpsmUpsfCmUfUmAfCmAfCmCfGm |
| siNA- | | UmGfUmAfAmGfAmCpsTpsT | | UfGmCfGmGfCmAfCTpsT |
| 109 | | | | |
| ds- | 4492 | fUpsmApsfCmCfAmCfAmUfAmUfAm | 4714 | mUpsfGpsmAfCmGfUmGfAmUfAmUf |
| siNA- | | UfCmAfCmGfUmCfATpsT | | AmUfGmUfGmGfUmApsTpsT |
| 110 | | | | |
| ds- | 4493 | mGpsfCpsmUfAmUfGmUfAmAfGmU | 4715 | fGpsmGpsfAmUfGmUfAmAfAmCfUm |
| siNA- | | fUmUfAmCfAmUfCmCpsTpsT | | UfAmCfAmUfAmGfCTpsT |
| 111 | | | | |
| ds- | 4494 | mApsfCpsmCfGmGfGmUfUmUfGmA | 4716 | fUpsmCpsfAmAfAmCfUmGfUmCfAm |
| siNA- | | fCmAfGmUfUmUfGmApsTpsT | | AfAmCfCmCfGmGfUTpsT |
| 112 | | | | |
| ds- | 4495 | fUpsmGpsfUmCfAmAfAmCfCmCfGm | 4717 | mApsfApsmAfAmUfUmAfCmCfGmGf |
| siNA- | | GfUmAfAmUfUmUfUTpsT | | GmUfUmUfGmAfCmApsTpsT |
| 113 | | | | |
| ds- | 4496 | fUpsmApsfAmGfUmAfUmGfCmCfA | 4718 | mUpsfGpsmCfAmCfUmAfAmUfGmGf |
| siNA- | | mUfUmAfGmUfGmCfATpsT | | CmAfUmAfCmUfUmApsTpsT |
| 114 | | | | |
| ds- | 4497 | mApsfUpsmUfCmUfUmUfGmCfAmCf | 4719 | fUpsmGpsfCmCfAmUfUmAfGmUfGm |
| siNA- | | UmAfAmUfGmGfCmApsTpsT | | CfAmAfAmGfAmAfUTpsT |
| 115 | | | | |
| ds- | 4498 | mGpsfCpsmGfAmGfCmUfCmUfAmUf | 4720 | fUpsmGpsfCmAfAmAfGmAfAmUfAm |
| siNA- | | UmCfUmUfUmGfCmApsTpsT | | GfAmGfCmUfCmGfCTpsT |
| 116 | | | | |
| ds- | 4499 | mGpsfApsmCfAmCfCmAfGmCfUmAf | 4721 | fUpsmCpsfGmCfAmCfCmGfUmAfGm |
| siNA- | | CmGfGmUfGmCfGmApsTpsT | | CfUmGfGmUfGmUfCTpsT |
| 117 | | | | |
| ds- | 4500 | fCpsmApsfAmUfAmGfCmCfGmCfCm | 4722 | mCpsfCpsmUfCmUfAmGfUmGfGmCf |
| siNA- | | AfCmUfAmGfAmGfGTpsT | | GmGfCmUfAmUfUmGpsTpsT |
| 118 | | | | |
| ds- | 4501 | mCpsfApsmCfUmAfUmUfAmGfCmAf | 4723 | fApsmCpsfUmGfCmUfUmAfUmGfCm |
| siNA- | | UmAfAmGfCmAfGmUpsTpsT | | UfAmAfUmAfGmUfGTpsT |
| 119 | | | | |
| ds- | 4502 | fApsmUpsfCmAfGmGfAmGfUmAfU | 4724 | mApsfCpsmAfUmCfAmGfCmAfUmAf |
| siNA- | | mGfCmUfGmAfUmGfUTpsT | | CmUfCmCfUmGfAmUpsTpsT |
| 120 | | | | |
| ds- | 4503 | mUpsfApsmAfUmAfGmCfUmCfAmU | 4725 | fUpsmApsfGmGfAmGfGmUfAmUfGm |
| siNA- | | fAmCfCmUfCmCfUmApsTpsT | | AfGmCfUmAfUmUfATpsT |
| 121 | | | | |
| ds- | 4504 | fApsmCpsfUmAfUmGfGmUfGmAfU | 4726 | mApsfCpsmAfAmCfAmGfCmAfUmCf |
| siNA- | | mGfCmUfGmUfUmGfUTpsT | | AmCfCmAfUmAfGmUpsTpsT |
| 122 | | | | |
| ds- | 4505 | fUpsmGpsfCmCfAmCfAmAfGmAfGm | 4727 | mApsfCpsmAfUmAfGmUfGmCfUmCf |
| siNA- | | CfAmCfUmAfUmGfUTpsT | | UmUfGmUfGmGfCmApsTpsT |
| 123 | | | | |
| ds- | 4506 | mUpsfApsmUfAmCfAmCfUmAfUmG | 4728 | fUpsmCpsfUmGfCmUfCmGfCmAfUm |
| siNA- | | fCmGfAmGfCmAfGmApsTpsT | | AfGmUfGmUfAmUfATpsT |
| 124 | | | | |
| ds- | 4507 | fApsmApsfUmUfCmAfAmAfGmUfG | 4729 | mGpsfUpsmUfGmAfAmUfUmCfAmCf |
| siNA- | | mAfAmUfUmCfAmAfCTpsT | | UmUfUmGfAmAfUmUpsTpsT |
| 125 | | | | |
| ds- | 4508 | mApsfGpsmGfAmAfCmAfUmGfUmC | 4730 | fUpsmApsfGmGfUmCfCmAfGmAfCm |
| siNA- | | fUmGfGmAfCmCfUmApsTpsT | | AfUmGfUmUfCmCfUTpsT |
| 126 | | | | |
| ds- | 4509 | mUpsfGpsmAfAmUfAmUfGmAfCmA | 4731 | fUpsmApsfUmGfAmCfUmAfUmGfUm |
| siNA- | | fUmAfGmUfCmAfUmApsTpsT | | CfAmUfAmUfUmCfATpsT |
| 127 | | | | |
| ds- | 4510 | fUpsmUpsfUmGfAmGfCmUfUmUfG | 4732 | mGpsfCpsmUfUmAfGmCfCmCfAmAf |
| siNA- | | mGfGmCfUmAfAmGfCTpsT | | AmGfCmUfCmAfAmApsTpsT |
| 128 | | | | |
| ds- | 4511 | mUpsfUpsmUfGmCfGmAfCmAfUmU | 4733 | fUpsmApsfAmUfGmAfUmGfAmAfUm |
| siNA- | | fCmAfUmCfAmUfUmApsTpsT | | GfUmCfGmCfAmAfATpsT |
| 129 | | | | |
| ds- | 4512 | fGpsmApsfGmUfAmCfGmAfAmCfU | 4734 | mApsfGpsmUfAmCfAmUfAmAfGmUf |
| siNA- | | mUfAmUfGmUfAmCfUTpsT | | UmCfGmUfAmCfUmCpsTpsT |
| 130 | | | | |
| ds- | 4513 | fGpsmGpsfUmAfCmGfUmUfAmAfU | 4735 | mUpsfApsmUfUmAfAmCfUmAfUmUf |
| siNA- | | mAfGmUfUmAfAmUfATpsT | | AmAfCmGfUmAfCmCpsTpsT |
| 131 | | | | |
| ds- | 4514 | mGpsfApsmAfAmAfAmGfAmAfGmU | 4736 | fApsmApsfUmAfGmCfGmUfAmCfUm |
| siNA- | | fAmCfGmCfUmAfUmUpsTpsT | | UfCmUfUmUfUmUfCTpsT |
| 132 | | | | |
| ds- | 4515 | mGpsfCpsmAfAmGfAmAfUmAfCmCf | 4737 | fGpsmCpsfUmUfUmCfGmUfGmGfUm |
| siNA- | | AmCfGmAfAmAfGmCpsTpsT | | AfUmUfCmUfUmGfCTpsT |
| 133 | | | | |
| ds- | 4516 | fCpsmUpsfUmAfCmUfGmCfGmCfUm | 4738 | mApsfCpsmAfAmUfCmGfAmAfGmCf |
| siNA- | | UfCmGfAmUfUmGfUTpsT | | GmCfAmGfUmAfAmGpsTpsT |
| 134 | | | | |
| ds- | 4517 | fUpsmUpsfCmUfGmGfUmCfUmAfA | 4739 | mUpsfApsmGfUmUfCmGfUmUfUmAf |
| siNA- | | mAfCmGfAmAfCmUfATpsT | | GmAfCmCfAmGfAmApsTpsT |
| 135 | | | | |
| ds- | 4518 | mUpsfApsmAfUmAfAmGfAmAfAmG | 4740 | fCpsmApsfCmGfAmAfCmGfCmUfUm |
| siNA- | | fCmGfUmUfCmGfUmGpsTpsT | | UfCmUfUmAfUmUfATpsT |
| 136 | | | | |
| ds- | 4519 | fUpsmCpsfAmGfGmUfUmUfUmGfC | 4741 | mUpsfGpsmUfAmUfGmCfAmGfCmAf |
| siNA- | | mUfGmCfAmUfAmCfATpsT | | AmAfAmCfCmUfGmApsTpsT |
| 137 | | | | |
| ds- | 4520 | mCpsfApsmUfCmUfGmUfUmGfUmCf | 4742 | fCpsmApsfGmUfAmAfGmUfGmAfCm |
| siNA- | | AmCfUmUfAmCfUmGpsTpsT | | AfAmCfAmGfAmUfGTpsT |
| 138 | | | | |
| ds- | 4521 | mGpsfApsmCfGmCfAmGfUmAfUmU | 4743 | fUpsmApsfCmCfCmAfAmUfAmAfUm |
| siNA- | | fAmUfUmGfGmGfUmApsTpsT | | AfCmUfGmCfGmUfCTpsT |
| 139 | | | | |
| ds- | 4522 | fApsmApsfAmUfUmGfGmCfUmAfC | 4744 | mCpsfUpsmUfCmGfGmUfAmGfUmAf |
| siNA- | | mUfAmCfCmGfAmAfGTpsT | | GmCfCmAfAmUfUmUpsTpsT |
| 140 | | | | |
| ds- | 4523 | fCpsmApsfAmAfAmGfGmCfUmUfCm | 4745 | mUpsfCpsmUfGmCfGmUfAmGfAmAf |
| siNA- | | UfAmCfGmCfAmGfATpsT | | GmCfCmUfUmUfUmGpsTpsT |
| 141 | | | | |
| ds- | 4524 | fUpsmGpsfUmCfAmCfUmAfAmGfA | 4746 | mApsfGpsmCfAmGfAmUfUmUfCmUf |
| siNA- | | mAfAmUfCmUfGmCfUTpsT | | UmAfGmUfGmAfCmApsTpsT |
| 142 | | | | |
| ds- | 4525 | mUpsfUpsmGfUmAfAmUfCmAfGmU | 4747 | fGpsmApsfCmAfAmGfGmAfAmCfUm |
| siNA- | | fUmCfCmUfUmGfUmCpsTpsT | | GfAmUfUmAfCmAfATpsT |
| 143 | | | | |
| ds- | 4526 | fCpsmApsfUmGfGmAfAmGfUmCfA | 4748 | mCpsfGpsmAfAmGfGmUfGmUfGmAf |
| siNA- | | mCfAmCfCmUfUmCfGTpsT | | CmUfUmCfCmAfUmGpsTpsT |
| 144 | | | | |
| ds- | 4527 | fUpsmCpsfUmAfCmUfCmUfUmGfUm | 4749 | mCpsfApsmUfUmCfUmGfCmAfCmAf |
| siNA- | | GfCmAfGmAfAmUfGTpsT | | AmGfAmGfUmAfGmApsTpsT |
| 145 | | | | |
| ds- | 4528 | fCpsmApsfCmAfUmAfGmCfAmAfUm | 4750 | mGpsfApsmUfUmAfAmAfGmAfUmUf |
| siNA- | | CfUmUfUmAfAmUfCTpsT | | GmCfUmAfUmGfUmGpsTpsT |
| 146 | | | | |
| ds- | 4529 | fUpsmGpsfAmAfAmGfAmGfCmCfA | 4751 | mApsfApsmAfUmGfUmGfGmUfGmGf |
| siNA- | | mCfCmAfCmAfUmUfUTpsT | | CmUfCmUfUmUfCmApsTpsT |
| 147 | | | | |
| ds- | 4530 | fApsmApsfGmAfGmCfCmCfUmAfAm | 4752 | mUpsfUpsmUfAmCfAmCfAmUfUmAf |
| siNA- | | UfGmUfGmUfAmAfATpsT | | GmGfGmCfUmCfUmUpsTpsT |
| 148 | | | | |
| ds- | 4531 | mUpsfGpsmCfAmAfCmAfCmGfGmAf | 4753 | fUpsmApsfCmGfGmUfUmUfCmGfUm |
| siNA- | | CmGfAmAfAmCfCmGfUmApsTpsT | | CfCmGfUmGfUmUfGmCpsfA |
| 149 | | | | |
| ds- | 4532 | fApsmApsfAmCfAmCfAmCfGmUfCm | 4754 | mApsfApsmCfUmGfAmGfUmUfGmGf |
| siNA- | | CfAmAfCmUfCmAfGmUpsfU | | AmCfGmUfGmUfGmUfUmUpsTpsT |
| 150 | | | | |
| ds- | 4533 | mApsfUpsmGfAmAfCmAfCmAfUmA | 4755 | fUpsmGpsfAmAfCmAfGmCfCmCfUm |
| siNA- | | fGmGfGmCfUmGfUmUfCmApsTpsT | | AfUmGfUmGfUmUfCmApsfU |
| 151 | | | | |
| ds- | 4534 | fUpsmApsfUmUfUmAfAmAfAmCfU | 4756 | mApsfUpsmUfGmUfCmAfGmUfAmAf |
| siNA- | | mUfAmCfUmGfAmCfAmApsfU | | GmUfUmUfUmAfAmAfUmApsTpsT |
| 152 | | | | |
| ds- | 4535 | fCpsmUpsfCmUfGmAfAmGfAmAfG | 4757 | mUpsfUpsmUfUmCfCmAfCmUfAmCf |
| siNA- | | mUfAmGfUmGfGmAfAmApsfA | | UmUfCmUfUmCfAmGfAmGpsTpsT |
| 153 | | | | |
| ds- | 4536 | fGpsmUpsfGmGfUmAfGmUfGmUfU | 4758 | mUpsfGpsmUfUmAfAmAfAmCfCmAf |
| siNA- | | mGfGmUfUmUfUmAfAmCpsfA | | AmCfAmCfUmAfCmCfAmCpsTpsT |
| 154 | | | | |
| ds- | 4537 | fApsmCpsfCmAfCmCfUmUfGmUfAm | 4759 | mGpsfUpsmAfAmCfAmAfAmCfCmUf |
| siNA- | | GfGmUfUmUfGmUfUmApsfC | | AmCfAmAfGmGfUmGfGmUpsTpsT |
| 155 | | | | |
| ds- | 4538 | mUpsfApsmCfCmAfGmUfGmUfGmU | 4760 | fUpsmGpsfUmUfGmUfGmUfAmCfAm |
| siNA- | | fGmUfAmCfAmCfAmAfCmApsTpsT | | CfAmCfAmCfUmGfGmUpsfA |
| 156 | | | | |
| ds- | 4539 | mApsfCpsmAfAmCfUmAfCmAfGmCf | 4761 | fApsmApsfAmGfGmUfUmAfUmGfGm |
| siNA- | | CmAfUmAfAmCfCmUfUmUpsTpsT | | CfUmGfUmAfGmUfUmGpsfU |
| 157 | | | | |
| ds- | 4540 | fUpsmUpsfAmAfAmCfGmGfGmUfU | 4762 | mUpsfUpsmAfCmAfCmCfGmCfAmAf |
| siNA- | | mUfGmCfGmGfUmGfUmApsfA | | AmCfCmCfGmUfUmUfAmApsTpsT |
| 158 | | | | |
| ds- | 4541 | mUpsfApsmAfGmAfCmGfGmGfCmU | 4763 | fGpsmUpsfGmUfAmAfGmUfGmCfAm |
| siNA- | | fGmCfAmCfUmUfAmCfAmCpsTpsT | | GfCmCfCmGfUmCfUmUpsfA |
| 159 | | | | |
| ds- | 4542 | fApsmCpsfAmUfGmGfUmAfCmCfAm | 4764 | mGpsfUpsmGfAmUfAmUfAmUfGmUf |
| siNA- | | CfAmUfAmUfAmUfCmApsfC | | GmGfUmAfCmCfAmUfGmUpsTpsT |
| 160 | | | | |
| ds- | 4543 | fUpsmCpsfAmAfAmCfUmGfUmCfAm | 4765 | mUpsfUpsmAfCmCfGmGfGmUfUmUf |
| siNA- | | AfAmCfCmCfGmGfUmApsfA | | GmAfCmAfGmUfUmUfGmApsTpsT |
| 161 | | | | |
| ds- | 4544 | mUpsfGpsmAfGmCfAmAfAmGfAmA | 4766 | fApsmApsfAmAfCmAfCmUfUmCfUm |
| siNA- | | fGmAfAmGfUmGfUmUfUmUpsTpsT | | UfCmUfUmUfGmCfUmCpsfA |
| 162 | | | | |
| ds- | 4545 | fUpsmUpsfGmAfUmUfGmUfUmAfC | 4767 | mApsfGpsmCfCmAfCmCfAmUfCmGf |
| siNA- | | mGfAmUfGmGfUmGfGmCpsfU | | UmAfAmCfAmAfUmCfAmApsTpsT |
| 163 | | | | |
| ds- | 4546 | fApsmApsfAmUfGmAfAmUfCmUfU | 4768 | mUpsfGpsmGfCmAfUmAfCmUfUmAf |
| siNA- | | mAfAmGfUmAfUmGfCmCpsfA | | AmGfAmUfUmCfAmUfUmUpsTpsT |
| 164 | | | | |
| ds- | 4547 | fUpsmApsfUmGfCmCfAmUfUmAfG | 4769 | mApsfUpsmUfCmUfUmUfGmCfAmCf |
| siNA- | | mUfGmCfAmAfAmGfAmApsfU | | UmAfAmUfGmGfCmAfUmApsTpsT |
| 165 | | | | |
| ds- | 4548 | fUpsmGpsfCmCfAmUfUmAfGmUfG | 4770 | mCpsfUpsmAfUmUfCmUfUmUfGmCf |
| siNA- | | mCfAmAfAmGfAmAfUmApsfG | | AmCfUmAfAmUfGmGfCmApsTpsT |
| 166 | | | | |
| ds- | 4549 | fApsmGpsfAmAfUmAfGmAfGmCfU | 4771 | mCpsfUpsmAfCmGfGmUfGmCfGmAf |
| siNA- | | mCfGmCfAmCfCmGfUmApsfG | | GmCfUmCfUmAfUmUfCmUpsTpsT |
| 167 | | | | |
| ds- | 4550 | mCpsfApsmGfCmUfAmCfGmGfUmGf | 4772 | fApsmUpsfAmGfAmGfCmUfCmGfCm |
| siNA- | | CmGfAmGfCmUfCmUfAmUpsTpsT | | AfCmCfGmUfAmGfCmUpsfG |
| 168 | | | | |
| ds- | 4551 | fUpsmCpsfGmCfAmCfCmGfUmAfGm | 4773 | mGpsfApsmGfAmCfAmCfCmAfGmCf |
| siNA- | | CfUmGfGmUfGmUfCmUpsfC | | UmAfCmGfGmUfGmCfGmApsTpsT |
| 169 | | | | |
| ds- | 4552 | fApsmApsfCmUfGmCfUmUfAmUfG | 4774 | mApsfCpsmAfCmUfAmUfUmAfGmCf |
| siNA- | | mCfUmAfAmUfAmGfUmGpsfU | | AmUfAmAfGmCfAmGfUmUpsTpsT |
| 170 | | | | |
| ds- | 4553 | fGpsmApsfCmUfGmAfGmAfCmUfG | 4775 | mUpsfUpsmAfGmUfAmAfGmGfUmCf |
| siNA- | | mAfCmCfUmUfAmCfUmApsfA | | AmGfUmCfUmCfAmGfUmCpsTpsT |
| 171 | | | | |
| ds- | 4554 | fGpsmApsfGmUfAmCfAmCfCmUfUm | 4776 | mApsfCpsmCfUmUfUmUfUmCfAmAf |
| siNA- | | UfGmAfAmAfAmAfGmGpsfU | | AmGfGmUfGmUfAmCfUmCpsTpsT |
| 172 | | | | |
| ds- | 4555 | fUpsmGpsfGmUfAmCfUmGfGmUfA | 4777 | mApsfApsmAfUmGfAmCfUmCfUmUf |
| siNA- | | mAfGmAfGmUfCmAfUmUpsfU | | AmCfCmAfGmUfAmCfCmApsTpsT |
| 173 | | | | |
| ds- | 4556 | fUpsmCpsfUmGfCmUfAmAfUmCfUm | 4778 | mApsfGpsmUfAmGfCmAfGmCfAmAf |
| siNA- | | UfGmCfUmGfCmUfAmCpsfU | | GmAfUmUfAmGfCmAfGmApsTpsT |
| 174 | | | | |
| ds- | 4557 | fGpsmUpsfAmCfGmUfUmAfAmUfA | 4779 | mCpsfGpsmCfUmAfUmUfAmAfCmUf |
| siNA- | | mGfUmUfAmAfUmAfGmCpsfG | | AmUfUmAfAmCfGmUfAmCpsTpsT |
| 175 | | | | |
| ds- | 4558 | fUpsmUpsfCmUfUmGfCmUfUmUfCm | 4780 | mApsfGpsmAfAmUfAmCfCmAfCmGf |
| siNA- | | GfUmGfGmUfAmUfUmCpsfU | | AmAfAmGfCmAfAmGfAmApsTpsT |
| 176 | | | | |
| ds- | 4559 | fCpsmUpsfUmAfCmUfGmCfGmCfUm | 4781 | mApsfCpsmAfCmAfAmUfCmGfAmAf |
| siNA- | | UfCmGfAmUfUmGfUmGpsfU | | GmCfGmCfAmGfUmAfAmGpsTpsT |
| 177 | | | | |
| ds- | 4560 | fCpsmGpsfCmUfUmCfGmAfUmUfGm | 4782 | mApsfGpsmUfAmCfGmCfAmCfAmCf |
| siNA- | | UfGmUfGmCfGmUfAmCpsfU | | AmAfUmCfGmAfAmGfCmGpsTpsT |
| 178 | | | | |
| ds- | 4561 | fCpsmGpsfUmAfCmUfGmCfUmGfCm | 4783 | mUpsfApsmAfCmAfAmUfAmUfUmGf |
| siNA- | | AfAmUfAmUfUmGfUmUpsfA | | CmAfGmCfAmGfUmAfCmGpsTpsT |
| 179 | | | | |
| ds- | 4562 | mUpsfCpsmGfUmUfUmAfGmAfCmCf | 4784 | fCpsmUpsfGmAfUmCfUmUfCmUfGm |
| siNA- | | AmGfAmAfGmAfUmCfAmGpsTpsT | | GfUmCfUmAfAmAfCmGpsfA |
| 180 | | | | |
| ds- | 4563 | mGpsfUpsmAfAmUfAmAfGmAfAmA | 4785 | fUpsmCpsfAmCfGmAfAmCfGmCfUm |
| siNA- | | fGmCfGmUfUmCfGmUfGmApsTpsT | | UfUmCfUmUfAmUfUmApsfC |
| 181 | | | | |
| ds- | 4564 | mApsfApsmUfAmAfUmUfUmUfCmA | 4786 | fUpsmApsfAmAfCmGfAmAfCmAfUm |
| siNA- | | fUmGfUmUfCmGfUmUfUmApsTpsT | | GfAmAfAmAfUmUfAmUpsfU |
| 182 | | | | |
| ds- | 4565 | mApsfApsmGfCmAfCmAfAmAfUmA | 4787 | fApsmApsfUmUfGmAfCmUfUmCfUm |
| siNA- | | fGmAfAmGfUmCfAmAfUmUpsTpsT | | AfUmUfUmGfUmGfCmUpsfU |
| 183 | | | | |
| ds- | 4566 | mApsfApsmCfCmAfAmGfAmCfGmCf | 4788 | fCpsmApsfAmUfAmAfUmAfCmUfGm |
| siNA- | | AmGfUmAfUmUfAmUfUmGpsTpsT | | CfGmUfCmUfUmGfGmUpsfU |
| 184 | | | | |
| ds- | 4567 | fApsmUpsfUmGfGmCfUmAfCmUfA | 4789 | mApsfGpsmCfUmCfUmUfCmGfGmUf |
| siNA- | | mCfCmGfAmAfGmAfGmCpsfU | | AmGfUmAfGmCfCmAfAmUpsTpsT |
| 185 | | | | |
| ds- | 4568 | fCpsmGpsfUmGfCmUfAmCfAmAfCm | 4790 | mCpsfCpsmUfUmGfAmGfGmAfAmGf |
| siNA- | | UfUmCfCmUfCmAfAmGpsfG | | UmUfGmUfAmGfCmAfCmGpsTpsT |
| 186 | | | | |
| ds- | 4569 | fGpsmApsfUmUfGmAfAmCfCmAfG | 4791 | mUpsfGpsmCfUmCfUmCfAmAfGmCf |
| siNA- | | mCfUmUfGmAfGmAfGmCpsfA | | UmGfGmUfUmCfAmAfUmCpsTpsT |
| 187 | | | | |
| ds- | 4570 | fUpsmGpsfGmUfAmAfAmGfGmCfC | 4792 | mUpsfGpsmUfUmGfUmUfGmUfUmGf |
| siNA- | | mAfAmCfAmAfCmAfAmCpsfA | | GmCfCmUfUmUfAmCfCmApsTpsT |
| 188 | | | | |
| ds- | 4571 | mGpsfCpsmCfUmCfAmGfCmAfGmCf | 4793 | fUpsmApsfAmGfAmAfAmUfCmUfGm |
| siNA- | | AmGfAmUfUmUfCmUfUmApsTpsT | | CfUmGfCmUfGmAfGmGpsfC |
| 189 | | | | |
| ds- | 4572 | fApsmGpsfAmCfAmAfGmGfAmAfC | 4794 | mUpsfUpsmUfGmUfAmAfUmCfAmGf |
| siNA- | | mUfGmAfUmUfAmCfAmApsfA | | UmUfCmCfUmUfGmUfCmUpsTpsT |
| 190 | | | | |
| ds- | 4573 | fApsmApsfAmUfUmGfGmAfUmGfA | 4795 | mUpsfGpsmGfAmUfCmUfUmUfGmUf |
| siNA- | | mCfAmAfAmGfAmUfCmCpsfA | | CmAfUmCfCmAfAmUfUmUpsTpsT |
| 191 | | | | |
| ds- | 4574 | fCpsmApsfCmCfAmAfCmAfGmAfGm | 4796 | mCpsfCpsmUfUmUfUmUfAmGfGmCf |
| siNA- | | CfCmUfAmAfAmAfAmGpsfG | | UmCfUmGfUmUfGmGfUmGpsTpsT |
| 192 | | | | |
| ds- | 4575 | mApsfUpsmUfCmAfUmUfCmUfGmCf | 4797 | fCpsmUpsfAmCfUmCfUmUfGmUfGm |
| siNA- | | AmCfAmAfGmAfGmUfAmGpsTpsT | | CfAmGfAmAfUmGfAmApsfU |
| 193 | | | | |
| ds- | 4576 | mUpsfCpsmGfAmUfCmGfUmAfCmUf | 4798 | fGpsmGpsfCmCfAmCfGmCfGmGfAm |
| siNA- | | CmCfGmCfGmUfGmGfCmCpsTpsT | | GfUmAfCmGfAmUfCmGpsfA |
| 194 | | | | |
| ds- | 4577 | mUpsfUpsmUfUmAfCmAfCmAfUmU | 4799 | fGpsmApsfAmGfAmGfCmCfCmUfAm |
| siNA- | | fAmGfGmGfCmUfCmUfUmCpsTpsT | | AfUmGfUmGfUmAfAmApsfA |
| 195 | | | | |
| ds- | 4578 | mApsmGpsmAmCfAmAmGmGfAfAf | 4800 | vmUpsfUpsmUmGmUmAmAmUmCm |
| siNA- | | CmUmGfAmUmUmAmCfAmApsmA | | AmGmUmUfCmCmUmUmGmUmCm |
| 196 | | | | UpsTpsT |
| ds- | 4579 | fApsmApsfCmUfGmCfUmUfAmUfG | 4801 | vmApsfCpsmAfCmUfAmUfUmAfGmC |
| siNA- | | mCfUmAfAmUfAmGfUmGpsfU | | fAmUfAmAfGmCfAmGfUmUpsTpsT |
| 197 | | | | |
| ds- | 4580 | fUpsmUpsfCmUfUmGfCmUfUmUfCm | 4802 | vmApsfGpsmAfAmUfAmCfCmAfCmG |
| siNA- | | GfUmGfGmUfAmUfUmCpsfU | | fAmAfAmGfCmAfAmGfAmApsTpsT |
| 198 | | | | |
| ds- | 4581 | fCpsmUpsfAmCfUmCfUmUfGmUfGm | 4803 | vmApsfUpsmUfCmAfUmUfCmUfGmC |
| siNA- | | CfAmGfAmAfUmGfAmApsfU | | fAmCfAmAfGmAfGmUfAmGpsTpsT |
| 199 | | | | |
| ds- | 4582 | fUpsmUpsfGmAfAmGfGmUfGmUfC | 4804 | mCpsfApsmAfAmCfAmGfAmGfAmCf |
| siNA- | | mUfCmUfGmUfUmUfGTpsT | | AmCfCmUfUmCfAmApsTpsT |
| 200 | | | | |
| ds- | 4583 | mApsfApsmGfUmCfAmAfCmAfCmCf | 4805 | fUpsmUpsfGmUfUmAfAmUfGmGfUm |
| siNA- | | AmUfUmAfAmCfAmApsTpsT | | GfUmUfGmAfCmUfUTpsT |
| 201 | | | | |
| ds- | 4584 | mUpsfCpsmAfAmCfUmGfUmUfGmCf | 4806 | fUpsmUpsfAmCfUmUfUmUfGmCfAm |
| siNA- | | AmAfAmAfGmUfAmApsTpsT | | AfCmAfGmUfUmGfATpsT |
| 202 | | | | |
| ds- | 4585 | mUpsfCpsmAfUmAfAmCfAmAfAmCf | 4807 | fApsmUpsfGmCfUmGfGmUfGmUfUm |
| siNA- | | AmCfCmAfGmCfAmUpsTpsT | | UfGmUfUmAfUmGfATpsT |
| 203 | | | | |
| ds- | 4586 | mGpsfUpsmAfGmUfUmGfCmAfUmC | 4808 | fCpsmUpsfUmCfUmGfGmUfGmAfUm |
| siNA- | | fAmCfCmAfGmAfAmGpsTpsT | | GfCmAfAmCfUmAfCTpsT |
| 204 | | | | |
| ds- | 4587 | fUpsmGpsfCmUfCmAfCmAfGmCfAm | 4809 | mGpsfCpsmAfUmAfGmUfGmUfGmCf |
| siNA- | | CfAmCfUmAfUmGfCTpsT | | UmGfUmGfAmGfCmApsTpsT |
| 205 | | | | |
| ds- | 4588 | fApsmGpsfAmUfGmCfGmGfUmGfA | 4810 | mGpsfUpsmAfAmAfCmAfAmUfCmAf |
| siNA- | | mUfUmGfUmUfUmAfCTpsT | | CmCfGmCfAmUfCmUpsTpsT |
| 206 | | | | |
| ds- | 4589 | mApsfUpsmCfUmAfCmUfUmUfCmUf | 4811 | fUpsmGpsfUmGfUmUfUmGfAmGfAm |
| siNA- | | CmAfAmAfCmAfCmApsTpsT | | AfAmGfUmAfGmAfUTpsT |
| 207 | | | | |
| ds- | 4590 | fUpsmUpsfUmAfAmUfGmUfUmAfG | 4812 | mApsfApsmGfCmAfUmCfAmCfUmAf |
| siNA- | | mUfGmAfUmGfCmUfUTpsT | | AmCfAmUfUmAfAmApsTpsT |
| 208 | | | | |
| ds- | 4591 | fApsmCpsfUmAfAmAfCmUfUmCfCm | 4813 | mCpsfGpsmUfAmAfUmAfAmGfGmAf |
| siNA- | | UfUmAfUmUfAmCfGTpsT | | AmGfUmUfUmAfGmUpsTpsT |
| 209 | | | | |
| ds- | 4592 | mUpsfUpsmUfGmUfAmAfUmCfAmG | 4814 | fApsmGpsfAmCfAmAfGmGfAmAfCm |
| siNA- | | fUmUfCmCfUmUfGmUfCmUpsTpsT | | UfGmAfUmUfAmCfAmAfATpsT |
| 210 | | | | |
| ds- | 4593 | fCpsmUpsfUmAfCmUfGmCfGmCfUm | 4815 | mApsfCpsmAfAmUfCmGfAmAfGmCf |
| siNA- | | UfCmGfAmUfUmGfUTpsT | | GmCfAmGfUmAfAmGpsTpsT |
| 211 | | | | |
| ds- | 4594 | mApsfCpsmAfCmUfAmUfUmAfGmCf | 4816 | fApsmApsfCmUfGmCfUmUfAmUfGm |
| siNA- | | AmUfAmAfGmCfAmGfUmUpsTpsT | | CfUmAfAmUfAmGfUmGfUTpsT |
| 212 | | | | |
| ds- | 4595 | mApsfGpsmAfAmUfAmCfCmAfCmGf | 4817 | fUpsmUpsfCmUfUmGfCmUfUmUfCm |
| siNA- | | AmAfAmGfCmAfAmGfAmApsTpsT | | GfUmGfGmUfAmUfUmCfUTpsT |
| 213 | | | | |
| ds- | 4596 | mApsfCpsmAfCmAfAmUfCmGfAmAf | 4818 | fCpsmUpsfUmAfCmUfGmCfGmCfUm |
| siNA- | | GmCfGmCfAmGfUmAfAmGpsTpsT | | UfCmGfAmUfUmGfUmGfUTpsT |
| 214 | | | | |
| ds- | 4597 | fCpsmUpsfAmCfUmCfUmUfGmUfGm | 4819 | mApsfUpsmUfCmAfUmUfCmUfGmCf |
| siNA- | | CfAmGfAmAfUmGfAmAfUTpsT | | AmCfAmAfGmAfGmUfAmGpsTpsT |
| 215 | | | | |
| ds- | 4598 | fUpsmUpsfUmCfUmCfAmAfCmUfAm | 4820 | mApsfGpsmGfAmAfGmUfUmUfAmGf |
| siNA- | | AfAmCfUmUfCmCfUpsTpsT | | UmUfGmAfGmAfAmApsTpsT |
| 216 | | | | |
| ds- | 4599 | mApsmGpsmAmCfAmAmGmGfAfAf | 4821 | vmUpsfUpsmUmGmUmAmAmUmCm |
| siNA- | | CmUmGfAmUmUmAmCfAmAmA | | AmGmUmUfCmCmUmUmGmUmCm |
| 217 | | | | UpsTpsT |
| ds- | 4600 | mApsmGpsmAmCmAmAfGmGfAfAf | 4822 | vmUpsfUpsmUmGmUfAmAmUmCmA |
| siNA- | | CmUmGmAmUmUmAmCmAmAmA | | mGmUmUfCmCfUmUmGmUmCmUps |
| 218 | | | | mUpsmU |
| ds- | 4601 | mApsmGpsmAmCmAmAfGmGfAfAf | 4823 | vmUpsfUpsmUmGfUmAmAfUmCmA |
| siNA- | | CmUmGmAmUmUmAmCmAmAmA | | mGmUmUfCmCmUfUmGmUfCmUps |
| 219 | | | | mUpsmU |
| ds- | 4602 | fCpsmUpsfAmCfUmCfUmUfGmUfGm | 4824 | vmApsfUpsmUfCmAfUmUfCmUfGmC |
| siNA- | | CfAmGfAmAfUmGfAmAfU | | fAmCfAmAfGmAfGmUfAmGpsTpsT |
| 220 | | | | |
| ds- | 4603 | mCpsmUpsmAmCmUmCfUmUfGfUf | 4825 | vmApsfUpsmUmCmAfUmUmCmUmG |
| siNA- | | GmCmAmGmAmAmUmGmAmAmU | | mCmAmCfAmAfGmAmGmUmAmGps |
| 221 | | | | mUpsmU |
| ds- | 4604 | mCpsmUpsmAmCmUmCfUmUfGfUf | 4826 | vmApsfUpsmUmCfAmUmUfCmUmG |
| siNA- | | GmCmAmGmAmAmUmGmAmAmU | | mCmAmCfAmAmGfAmGmUfAmGps |
| 222 | | | | mUpsmU |
| ds- | 4827 | mApsmGpsmAmCfAmAmGmGfAfAf | 4829 | vmUpsfUpsmUmGmUmAmAmUmCm |
| siNA- | | CmUmGfAmUmUmAmCfApsmApsm | | AmGmUmUfCmCmUmUmGmUmCm |
| 223 | | A | | UpsmUpsmU |
| ds- | 4828 | fCpsmUpsfAmCfUmCfUmUfGmUfGm | 4830 | vmApsfUpsmUfCmAfUmUfCmUfGmC |
| siNA- | | CfAmGfAmAfUmGfApsmApsfU | | fAmCfAmAfGmAfGmUfAmGpsmUps |
| 224 | | | | mU |

mX = 2′-O-methyl nucleotide;
fX = 2′-fluoro nucleotide;
ps = phosphorothioate linkage;
vX = 5′ vinyl phosphonate nucleotide;
vmX = 5′ vinyl phosphonate, 2′-O-methyl nucleotide

In some embodiments, a siNA of the present disclosure may comprise a sense strand selected from any one of SEQ ID NOs: 4383 to 4604, 4827, and 4828. In some embodiments, a siNA of the present disclosure may comprise an antisense strand selected from any one of SEQ ID NOs: 4605 to 4826, 4829, and 4830. In some embodiments, a siNA of the present disclosure may comprise a sense strand selected from any one of SEQ ID NOs: 4383 to 4604, 4827, and 4828 and an antisense strand selected from any one of SEQ ID NOs: 4605 to 4826, 4829, and 4830. In some embodiments, a siNA of the present disclosure may comprise a sense strand and an antisense strand, respectively, selected from SEQ ID NOs:

4383 and 4605;
4384 and 4606;
4385 and 4607;
4386 and 4608;
4387 and 4609;
4388 and 4610;
4389 and 4611;
4390 and 4612;
4391 and 4613;
4392 and 4614;
4393 and 4615;
4394 and 4616;
4395 and 4617;
4396 and 4618;
4397 and 4619;
4398 and 4620;
4399 and 4621;
4400 and 4622;
4401 and 4623;
4402 and 4624;
4403 and 4625;
4404 and 4626;
4405 and 4627;
4406 and 4628;
4407 and 4629;
4408 and 4630;
4409 and 4631;
4410 and 4632;
4411 and 4633;
4412 and 4634;
4413 and 4635;
4414 and 4636;
4415 and 4637;
4416 and 4638;
4417 and 4639;
4418 and 4640;
4419 and 4641;
4420 and 4642;
4421 and 4643;
4422 and 4644;
4423 and 4645;
4424 and 4646;
4425 and 4647;
4426 and 4648;
4427 and 4649;
4428 and 4650;
4429 and 4651;
4430 and 4652;
4431 and 4653;
4432 and 4654;
4433 and 4655;
4434 and 4656;
4435 and 4657;
4436 and 4658;
4437 and 4659;
4438 and 4660;
4439 and 4661;
4440 and 4662;
4441 and 4663;
4442 and 4664;
4443 and 4665;
4444 and 4666;
4445 and 4667;
4446 and 4668;
4447 and 4669;
4448 and 4670;
4449 and 4671;
4450 and 4672;
4451 and 4673;
4452 and 4674;
4453 and 4675;
4454 and 4676;
4455 and 4677;
4456 and 4678;
4457 and 4679;
4458 and 4680;
4459 and 4681;
4460 and 4682;
4461 and 4683;
4462 and 4684;
4463 and 4685;
4464 and 4686;
4465 and 4687;
4466 and 4688;
4467 and 4689;
4468 and 4690;
4469 and 4691;
4470 and 4692;
4471 and 4693;
4472 and 4694;
4473 and 4695;
4474 and 4696;
4475 and 4697;
4476 and 4698;
4477 and 4699;
4478 and 4700;
4479 and 4701;
4480 and 4702;
4481 and 4703;
4482 and 4704;
4483 and 4705;
4484 and 4706;
4485 and 4707;
4486 and 4708;
4487 and 4709;
4488 and 4710;
4489 and 4711;
4490 and 4712;
4491 and 4713;
4492 and 4714;
4493 and 4715;
4494 and 4716;
4495 and 4717;
4496 and 4718;
4497 and 4719;
4498 and 4720;
4499 and 4721;
4500 and 4722;
4501 and 4723;
4502 and 4724;
4503 and 4725;
4504 and 4726;
4505 and 4727;
4506 and 4728;
4507 and 4729;
4508 and 4730;
4509 and 4731;
4510 and 4732;
4511 and 4733;
4512 and 4734;
4513 and 4735;
4514 and 4736;
4515 and 4737;
4516 and 4738;
4517 and 4739;
4518 and 4740;
4519 and 4741;
4520 and 4742;
4521 and 4743;
4522 and 4744;
4523 and 4745;
4524 and 4746;
4525 and 4747;
4526 and 4748;
4527 and 4749;
4528 and 4750;
4529 and 4751;
4530 and 4752;
4531 and 4753;
4532 and 4754;
4533 and 4755;
4534 and 4756;
4535 and 4757;
4536 and 4758;
4537 and 4759;
4538 and 4760;
4539 and 4761;
4540 and 4762;
4541 and 4763;
4542 and 4764;
4543 and 4765;
4544 and 4766;
4545 and 4767;
4546 and 4768;
4547 and 4769;
4548 and 4770;
4549 and 4771;
4550 and 4772;
4551 and 4773;
4552 and 4774;
4553 and 4775;
4554 and 4776;
4555 and 4777;
4556 and 4778;
4557 and 4779;
4558 and 4780;
4559 and 4781;
4560 and 4782;
4561 and 4783;
4562 and 4784;
4563 and 4785;
4564 and 4786;
4565 and 4787;
4566 and 4788;
4567 and 4789;
4568 and 4790;
4569 and 4791;
4570 and 4792;
4571 and 4793;
4572 and 4794;
4573 and 4795;
4574 and 4796;
4575 and 4797;
4576 and 4798;
4577 and 4799;
4578 and 4800;
4579 and 4801;
4580 and 4802;
4581 and 4803;
4582 and 4804;
4583 and 4805;
4584 and 4806;
4585 and 4807;
4586 and 4808;
4587 and 4809;
4588 and 4810;
4589 and 4811;
4590 and 4812;
4591 and 4813;
4592 and 4814;
4593 and 4815;
4594 and 4816;
4595 and 4817;
4596 and 4818;
4597 and 4819;
4598 and 4820;
4599 and 4821;
4600 and 4822;
4601 and 4823;
4602 and 4824;
4603 and 4825;
4604 and 4826;
4827 and 4829; &
4828 and 4830.

In some embodiments, the siNA can be selected from ds-siNA-196 (sense and antisense respectively comprising SEQ ID NOs: 4578 and 4800), ds-siNA-197 (sense and antisense respectively comprising SEQ ID NOs: 4579 and 4801), ds-siNA-198 (sense and antisense respectively comprising SEQ ID NOs: 4580 and 4802), ds-siNA-199 (sense and antisense respectively comprising SEQ ID NOs: 4581 and 4803), ds-siNA-217 (sense and antisense respectively comprising SEQ ID NOs: 4599 and 4821), ds-siNA-218 (sense and antisense respectively comprising SEQ ID NOs: 4600 and 4822), ds-siNA-219 (sense and antisense respectively comprising SEQ ID NOs: 4601 and 4823), ds-siNA-220 (sense and antisense respectively comprising SEQ ID NOs: 4602 and 4824), ds-siNA-221 (sense and antisense respectively comprising SEQ ID NOs: 4603 and 4825), and ds-siNA-222 (sense and antisense respectively comprising SEQ ID NOs: 4604 and 4826).

In some embodiments, the siNA can be selected from ds-siNA-196 (sense and antisense respectively comprising SEQ ID NOs: 4578 and 4800), ds-siNA-197 (sense and antisense respectively comprising SEQ ID NOs: 4579 and 4801), ds-siNA-198 (sense and antisense respectively comprising SEQ ID NOs: 4580 and 4802), and ds-siNA-199 (sense and antisense respectively comprising SEQ ID NOs: 4581 and 4803). These siNA comprise a 5′-vinyl phosphonate and are derived from siRNAs that showed high potency in the live virus assay prior to the incorporation of the 5′-vinyl phosphonate. It was determined that the 5′-VP further improved potency for all constructs (see Examples). The most potent siNA were ds-siNA-196 and ds-siNA-199, which were selected for further modification.

In some embodiments, the siNA can be selected from, ds-siNA-217 (sense and antisense respectively comprising SEQ ID NOs: 4599 and 4821), ds-siNA-218 (sense and antisense respectively comprising SEQ ID NOs: 4600 and 4822), ds-siNA-219 (sense and antisense respectively comprising SEQ ID NOs: 4601 and 4823), ds-siNA-220 (sense and antisense respectively comprising SEQ ID NOs: 4602 and 4824), ds-siNA-221 (sense and antisense respectively comprising SEQ ID NOs: 4603 and 4825), and ds-siNA-222 (sense and antisense respectively comprising SEQ ID NOs: 4604 and 4826). These siNA are further modified forms of ds-siNA-196 and ds-siNA-199, which have different 2′-fluoro contents (three variants for each one of the parent siRNAs). All of these siNA also showed high potency across screening assays (see Examples).

Additionally, analogs of the specific embodiments (ds-siNA-001 to ds-siNA-224) can be prepared by altering or adjusting the modified nucleotides, phosphorylation blockers, 5′-stabilized end caps, and/or linkers as disclosed herein. For example, ds-siNA-223 is an analog of ds-siNA-196 in which an additional ps and mUmU overhang have been incorporated in place of dTdT. Similarly, ds-siNA-224 is an analog of ds-siNA-199 in which an additional ps and mUmU overhang have been incorporated in place of dTdT. Those skilled in the art will understand that other analogs can be similarly constructed.

Any of the foregoing specific embodiments can be incorporated into a pharmaceutical compositions, either alone or in combination with 1, 2, 3, 4, 5, 6, 7, 8, 9, or 10 or more additional siNA disclosed herein. Any of the foregoing specific embodiments can be used to treat or prevent viral infections, such as coronavirus infections (e.g., COVID-19) pursuant to the methods and uses disclosed herein.

### Pharmaceutical Compositions

The present disclosure also encompasses pharmaceutical compositions comprising siNAs of the present disclosure. One embodiment is a pharmaceutical composition comprising one or more siNA of the present disclosure, and a pharmaceutically acceptable diluent or carrier.

In some embodiments, the pharmaceutical compositions comprising any of the siNA molecules, sense strands, antisense strands, first nucleotide sequences, or second nucleotide sequences described herein. The compositions may comprise 1, 2, 3, 4, 5, 6, 7, 8, 9, 10, 11, 12 or more siNA molecules described herein. The compositions may comprise a first nucleotide sequence (i.e., a sense strand) comprising a nucleotide sequence of any one SEQ ID NOs: 1-1203, 2411-3392, 4383-4604, 4827, and 4828. In some embodiments, the composition comprises a second nucleotide sequence (i.e., antisense strand) comprising a nucleotide sequence of any one of SEQ ID NOs: 1204-2406, 3393-4374, 4605-4826, 4829, and 4830. In some embodiments, the composition comprises a sense strand comprising a nucleotide sequence of any one of SEQ ID NOs: 1-1203, 2411-3392, 4383-4604, 4827, and 4828. In some embodiments, the composition comprises an antisense strand comprising a nucleotide sequence of any one of SEQ ID NOs: 1204-2406, 3393-4374, 4605-4826, 4829, and 4830.

Alternatively or additionally, the pharmaceutical compositions may comprise (a) a phosphorylation blocker; and (b) a siNA. In some embodiments, the phosphorylation blocker is any of the phosphorylation blockers disclosed herein. In some embodiments, the siNA is any of the siNAs disclosed herein. In some embodiments, the siNA comprises any of the sense strands, antisense strands, first nucleotide sequences, or second nucleotide sequences described herein.

In some embodiments, the siNA comprises any of the sense strands, antisense strands, first nucleotide sequences, or second nucleotide sequences described herein. In some embodiments, the siNA comprises one or more modified nucleotides. In some embodiments, the one or more modified nucleotides are independently selected from a 2′-fluoro nucleotide and a 2′-O-methyl nucleotide. In some embodiments, the 2′-fluoro nucleotide or the 2′-O-methyl nucleotide is independently selected from any of the 2′-fluoro or 2′-O-methyl nucleotide mimics disclosed herein. In some embodiments, the siNA comprises a nucleotide sequence comprising any of the modification patterns disclosed herein. In some embodiments, the composition comprises (a) a conjugated moiety; and (b) a short interfering nucleic acid (siNA). In some embodiments, the siNA is any of the siNAs disclosed herein. In some embodiments, the siNA comprises any of the sense strands, antisense strands, first nucleotide sequences, or second nucleotide sequences described herein.

In some embodiments, the siNA comprises any of the sense strands, antisense strands, first nucleotide sequences, or second nucleotide sequences described herein. In some embodiments, the siNA comprises one or more modified nucleotides. In some embodiments, the one or more modified nucleotides are independently selected from a 2′-fluoro nucleotide and a 2′-O-methyl nucleotide. In some embodiments, the 2′-fluoro nucleotide or the 2′-O-methyl nucleotide is independently selected from any of the 2′-fluoro or 2′-O-methyl nucleotide mimics disclosed herein. In some embodiments, the siNA comprises a nucleotide sequence comprising any of the modification patterns disclosed herein.

In some embodiments, the pharmaceutical composition comprises (a) a 5′-stabilized end cap; and (b) a siNA. In some embodiments, the 5′-stabilized end cap is any of the 5-stabilized end caps disclosed herein. In some embodiments, the siNA is any of the siNAs disclosed herein. In some embodiments, the siNA comprises any of the sense strands, antisense strands, first nucleotide sequences, or second nucleotide sequences described herein. In some embodiments, the siNA comprises one or more modified nucleotides. In some embodiments, the one or more modified nucleotides are independently selected from a 2′-fluoro nucleotide and a 2′-O-methyl nucleotide. In some embodiments, the 2′-fluoro nucleotide or the 2′-O-methyl nucleotide is independently selected from any of the 2′-fluoro or 2′-O-methyl nucleotide mimics disclosed herein. In some embodiments, the siNA comprises a nucleotide sequence comprising any of the modification patterns disclosed herein.

In some embodiments, the pharmaceutical composition comprises (a) at least one phosphorylation blocker, conjugated moiety, or 5′-stabilized end cap; and (b) a short interfering nucleic acid (siNA). In some embodiments, the phosphorylation blocker is any of the phosphorylation blockers disclosed herein. In some embodiments, the 5′-stabilized end cap is any of the 5-stabilized end caps disclosed herein. In some embodiments, the siNA is any of the siNAs disclosed herein. In some embodiments, the siNA comprises any of the sense strands, antisense strands, first nucleotide sequences, or second nucleotide sequences described herein. In some embodiments, the siNA comprises one or more modified nucleotides. In some embodiments, the one or more modified nucleotides are independently selected from a 2′-fluoro nucleotide and a 2′-O-methyl nucleotide. In some embodiments, the 2′-fluoro nucleotide or the 2′-O-methyl nucleotide is independently selected from any of the 2′-fluoro or 2′-O-methyl nucleotide mimics disclosed herein. In some embodiments, the siNA comprises a nucleotide sequence comprising any of the modification patterns disclosed herein.

In some embodiments, the pharmaceutical composition containing the siNA of the present disclosure is formulated for systemic administration via parenteral delivery. Parenteral administration includes intravenous, intra-arterial, subcutaneous, intraperitoneal or intramuscular injection or infusion; also subdermal administration, e.g., via an implanted device. In a preferred embodiment, the pharmaceutical composition containing the siNA of the present disclosure is formulated for subcutaneous (SC) or intravenous (IV) delivery. Formulations for parenteral administration may include sterile aqueous solutions, which may also contain buffers, diluents and other pharmaceutically acceptable additives as understood by the skilled artisan. For intravenous use, the total concentration of solutes may be controlled to render the preparation isotonic.

The pharmaceutical compositions containing the siNA of the present disclosure are useful for treating a disease or disorder, e.g., associated with the expression or activity of a coronavirus gene, more specifically a non-structural protein, such as nsp8, nsp9, nsp10, nsp11, nsp12, nsp13, nsp14, or nsp15.

In some embodiments, the pharmaceutical composition comprises a siNA of the present disclosure that is complementary or hybridizes to a viral target RNA sequence (e.g., a non-structural protein of coronavirus), and a pharmaceutically acceptable diluent or carrier. When the pharmaceutical composition comprises two or more siNAs, the siNAs may be present in varying amounts. For example, in some embodiments, the weight ratio of first siNA to second siNA is 1:4 to 4:1, e.g., 1:4, 1:3, 1:2, 1:1, 2:1, 3:1, or 4:1. In some embodiments, the molar ratio of first siNA to second siNA is 1:4 to 4:1, e.g., 1:4, 1:3, 1:2, 1:1, 2:1, 3:1, or 4:1.

In some embodiments, the pharmaceutical composition comprises an amount of one or more of the siNA molecules described herein formulated with one or more pharmaceutically acceptable carriers (additives) and/or diluents. The pharmaceutical compositions may be specially formulated for administration in solid or liquid form, including those adapted for the following: (1) parenteral administration, for example, by subcutaneous, intramuscular, intravenous or epidural injection as, for example, a sterile solution or suspension, or sustained-release formulation; (2) topical application, for example, as a cream, ointment, or a controlled-release patch or spray applied to the skin; (3) intravaginally or intrarectally, for example, as a pessary, cream or foam; (4) sublingually; (5) ocularly; (6) transdermally; or (7) nasally.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Formulations of the present disclosure include those suitable for nasal, topical (including buccal and sublingual), rectal, vaginal and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the compound (e.g., siNA molecule) which produces a therapeutic effect. Generally, out of one hundred percent, this amount will range from about 0.1 percent to about ninety-nine percent of active ingredient, preferably from about 5 percent to about 70 percent, most preferably from about 10 percent to about 30 percent.

In some embodiments, a formulation of the present disclosure comprises an excipient selected from the group consisting of cyclodextrins, celluloses, liposomes, micelle forming agents, e.g., bile acids, and polymeric carriers, e.g., polyesters and polyanhydrides; and a compound (e.g., siNA molecule) of the present disclosure.

Methods of preparing these formulations or compositions include the step of bringing into association a compound (e.g., siNA molecule) of the present disclosure with the carrier and, optionally, one or more accessory ingredients. In general, the formulations are prepared by uniformly and intimately bringing into association a compound (e.g., siNA molecule) of the present disclosure with liquid carriers, or finely divided solid carriers, or both, and then, if necessary, shaping the product.

Formulations of the disclosure suitable for a suspension in an aqueous or non-aqueous liquid, or as an oil-in-water or water-in-oil liquid emulsion, or as an elixir or syrup, each containing a predetermined amount of a compound (e.g., siNA molecule) of the present disclosure as an active ingredient. A compound (e.g., siNA molecule) of the present disclosure may also be administered as a bolus, electuary, or paste.

In dosage forms of the disclosure, the active ingredient may be mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds and surfactants, such as poloxamer and sodium lauryl sulfate; (7) wetting agents, such as, for example, cetyl alcohol, glycerol monostearate, and non-ionic surfactants; (8) absorbents, such as kaolin and bentonite clay; (9) lubricants, such as talc, calcium stearate, magnesium stearate, solid polyethylene glycols, sodium lauryl sulfate, zinc stearate, sodium stearate, stearic acid, and mixtures thereof; (10) coloring agents; and (11) controlled release agents such as crospovidone or ethyl cellulose.

The disclosed dosage forms may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms of the compounds (e.g., siNA molecules) of the disclosure include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (I particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the compositions can also include adjuvants such as wetting agents, emulsifying and suspending agents, sweetening, flavoring, coloring, perfuming and preservative agents.

Suspensions, in addition to the active compounds (e.g., siNA molecules), may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions of the disclosure for rectal or vaginal administration may be presented as a suppository, which may be prepared by mixing one or more compounds (e.g., siNA molecules) of the disclosure with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound (e.g., siNA molecule).

Formulations of the present disclosure which are suitable for vaginal administration also include pessaries, tampons, creams, gels, pastes, foams or spray formulations containing such carriers as are known in the art to be appropriate.

Dosage forms for the topical or transdermal administration of a compound (e.g., siNA molecule) of this disclosure include powders, sprays, ointments, pastes, creams, lotions, gels, solutions, patches and inhalants. The active compound (e.g., siNA molecule) may be mixed under sterile conditions with a pharmaceutically acceptable carrier, and with any preservatives, buffers, or propellants which may be required.

The ointments, pastes, creams and gels may contain, in addition to an active compound (e.g., siNA molecule) of this disclosure, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound (e.g., siNA molecule) of this disclosure, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound (e.g., siNA molecule) of the present disclosure to the body. Such dosage forms can be made by dissolving or dispersing the compound (e.g., siNA molecule) in the proper medium. Absorption enhancers can also be used to increase the flux of the compound (e.g., siNA molecule) across the skin. The rate of such flux can be controlled by either providing a rate controlling membrane or dispersing the compound (e.g., siNA molecule) in a polymer matrix or gel.

Ophthalmic formulations, eye ointments, powders, solutions and the like, are also contemplated as being within the scope of this invention.

Pharmaceutical compositions of this disclosure suitable for parenteral administration comprise one or more compounds (e.g., siNA molecules) of the disclosure in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the disclosure include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms upon the subject compounds may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of the subject compounds (e.g., siNA molecules) in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

When the compounds (e.g., siNA molecules) of the present disclosure are administered as pharmaceuticals, to humans and animals, they can be given per se or as a pharmaceutical composition containing, for example, 0.1 to 99% (more preferably, 10 to 30%) of active ingredient in combination with a pharmaceutically acceptable carrier.

### Methods of Treatment and Administration

The siNA molecules of the present disclosure may be used to treat or prevent a disease in a subject in need thereof. In some embodiments, a method of treating or preventing a disease in a subject in need thereof comprises administering to the subject any of the siNA molecules disclosed herein. In some embodiments, a method of treating or preventing a disease in a subject in need thereof comprises administering to the subject any of the compositions disclosed herein.

In some embodiments of the disclosed methods and uses, the disease is a respiratory disease. In some embodiments, the respiratory disease is a viral infection. In some embodiments, the respiratory disease is viral pneumonia. In some embodiments, the respiratory disease is an acute respiratory infection. In some embodiments, the respiratory disease is a cold. In some embodiments, the respiratory disease is severe acute respiratory syndrome (SARS). In some embodiments, the respiratory disease is Middle East respiratory syndrome (MERS). In some embodiments, the disease is coronavirus disease 2019 (e.g., COVID-19). In some embodiments, the respiratory disease can include one or more symptoms selected from coughing, sore throat, runny nose, sneezing, headache, fever, shortness of breath, myalgia, abdominal pain, fatigue, difficulty breathing, persistent chest pain or pressure, difficulty waking, loss of smell and taste, muscle or joint pain, chills, nausea or vomiting, nasal congestion, diarrhea, haemoptysis, conjunctival congestion, sputum production, chest tightness, and palpitations. In some embodiments, the respiratory disease can include complications selected from sinusitis, otitis media, pneumonia, acute respiratory distress syndrome, disseminated intravascular coagulation, pericarditis, and kidney failure. In some embodiments, the respiratory disease is idiopathic.

In some embodiments, the subject is a mammal. In some embodiments, the subject is a human. In some embodiments, the subject is a non-human primate. In some embodiments, the subject is a cat. In some embodiments, the subject is a camel. In preferred embodiments in which the subject is a human, the subject may be at least 40 years old, at least 45 years old, at least 50 years old, at least 55 years old, at least 60 years old, at least 65 years old, at least 70 years old, at least 75 years old, or at least 80 years old or older. In some embodiments, the subject is a pediatric subject (i.e., less than 18 years old).

The preparations (e.g., siNA molecules or pharmaceutical compositions thereof) of the present disclosure may be given parenterally, topically, or rectally or administered in the form of an inhalant. They are, of course, given in forms suitable for each administration route. For example, they are administered in tablets or capsule form, administration by injection, infusion, or inhalation; topical by lotion or ointment; rectal by suppositories. Injection, infusion, or inhalation are preferred.

These compounds may be administered to humans and other animals for therapy or as a prophylactic by any suitable route of administration, including nasally (as by, for example, a spray), rectally, intravaginally, parenterally, intracisternally and topically, as by powders, ointments or drops, including buccally and sublingually. In some embodiments, the compounds or compositions are inhaled, as by, for example, an inhaler, a nebulizer, or in an aerosolized form.

Regardless of the route of administration selected, the compounds (e.g., siNA molecules) of the present disclosure, which may be used in a suitable hydrated form, and/or the pharmaceutical compositions of the present disclosure, are formulated into pharmaceutically-acceptable dosage forms by conventional methods known to those of skill in the art.

In some embodiments, the present disclosure provides methods of treating or preventing a coronavirus infection, comprising administering to a subject in need thereof a therapeutically effective amount of one or more of the siNAs or a pharmaceutical composition as disclosed herein. In some embodiments, the coronavirus infection is selected from the group consisting of Middle East Respiratory Syndrome (MERS), Severe Acute Respiratory Syndrome (SARS), and COVID-19. In some embodiments, the subject has been treated with one or more additional coronavirus treatment agents. In some embodiments, the subject is concurrently treated with one or more additional coronavirus treatment agents.

Actual dosage levels of the active ingredients (e.g., siNA molecules) in the pharmaceutical compositions of this disclosure may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular compound (e.g., siNA molecule) of the present disclosure employed, or the ester, salt or amide thereof, the route of administration, the time of administration, the rate of excretion or metabolism of the particular compound being employed, the rate and extent of absorption, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds (e.g., siNA molecules) of the disclosure employed in the pharmaceutical composition at levels lower than that required in order to achieve the desired therapeutic effect and gradually increase the dosage until the desired effect is achieved.

In general, a suitable daily dose of a compound (e.g., siNA molecule) of the disclosure is the amount of the compound that is the lowest dose effective to produce a therapeutic effect. Such an effective dose generally depends upon the factors described above. Preferably, the compounds are administered at about 0.01 mg/kg to about 200 mg/kg, more preferably at about 0.1 mg/kg to about 100 mg/kg, even more preferably at about 0.5 mg/kg to about 50 mg/kg. In some embodiments, the compound is administered at a dose equal to or greater than 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, 0.25, 0.26, 0.27, 0.28, 0.29, 0.30, 0.35, 0.40, 0.45, 0.50, 0.55, 0.60, 0.65, 0.7, 0.75, 0.8, 0.85, 0.9, 0.95, or 1 mg/kg. In some embodiments, the compound is administered at a dose equal to or less than 200, 190, 180, 170, 160, 150, 140, 130, 120, 110, 100, 95, 90, 85, 80, 75, 70, 65, 60, 55, 50, 45, 40, 35, 30, 25, 20, or 15 mg/kg. In some embodiments, the total daily dose of the compound is equal to or greater than 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, or 100 mg.

If desired, the effective daily dose of the active compound (e.g., siNA) may be administered as two, three, four, five, six, seven, eight, nine, ten or more doses or sub-doses administered separately at appropriate intervals throughout the day, optionally, in unit dosage forms. In some embodiments, the compound is administered at least 1, 2, 3, 4, 5, 6, 7, 8, 9, 10, 11, 12, 13, 14, or 15 times. Preferred dosing is one administration per day. In some embodiments, the compound is administered at least 1, 2, 3, 4, 5, 6, 7, 8, 9, 10, 11, 12, 13, 14, 15, 16, 17, 18, 19, 20, or 21 times a week. In some embodiments, the compound is administered at least 1, 2, 3, 4, 5, 6, 7, 8, 9, 10, 11, 12, 13, 14, 15, 16, 17, 18, 19, 20, or 21 times a month. In some embodiments, the compound is administered once every 1, 2, 3, 4, 5, 6, 7, 8, 9, 10, 11, 12, 13, 14, 15, 16, 17, 18, 19, 20, or 21 days. In some embodiments, the compound is administered every 3 days. In some embodiments, the compound is administered once every 1, 2, 3, 4, 5, 6, 7, 8, 9, 10, 11, 12, 13, 14, or 15 weeks. In some embodiments, the compound is administered every month. In some embodiments, the compound is administered once every 1, 2, 3, 4, 5, 6, 7, 8, 9, 10, 11, 12, 13, 14, or 15 months. In some embodiments, the compound is administered at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, or 53 times over a period of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 days. In some embodiments, the compound is administered at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, or 53 times over a period of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, or 53 weeks. In some embodiments, the compound is administered at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, or 53 times over a period of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, or 53 months. In some embodiments, the compound is administered at least once a week for a period of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 weeks. In some embodiments, the compound is administered at least once a week for a period of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 months. In some embodiments, the compound is administered at least twice a week fora period of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 weeks. In some embodiments, the compound is administered at least twice a week for a period of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 months. In some embodiments, the compound is administered at least once every two weeks for a period of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 weeks. In some embodiments, the compound is administered at least once every two weeks for a period of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 months. In some embodiments, the compound is administered at least once every four weeks fora period of at least 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 weeks. In some embodiments, the compound is administered at least once every four weeks for a period of at least 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 months.

In some embodiments, any one of the siNAs or compositions disclosed herein is administered in a particle or viral vector. In some embodiments, the viral vector is a vector of adenovirus, adeno-associated virus (AAV), alphavirus, flavivirus, herpes simplex virus, lentivirus, measles virus, picornavirus, poxvirus, retrovirus, or rhabdovirus. In some embodiments, the viral vector is a recombinant viral vector. In some embodiments, the viral vector is selected from AAVrh.74, AAVrh.10, AAVrh.20, AAV-1, AAV-2, AAV-3, AAV-4, AAV-5, AAV-6, AAV-7, AAV-8, AAV-9, AAV-10, AAV-11, AAV-12 and AAV-13.

The subject of the described methods may be a mammal, and it includes humans and non-human mammals. In some embodiments, the subject is a human, such as an adult human.

The disclosed siNA can be administered alone or in combination with one or more additional coronavirus treatment agents and/or antiviral agents. The additional coronavirus treatment agent and/or antiviral may be a small molecule (e.g., a nucleoside analog or a protease inhibitor) or a biologic (e.g., an antibody or peptide). Examples of suitable coronavirus treatment agents include, but are not limited to, remdesivir, favipiravir, molnupiravir, dexamethasone, bamlanivimab, casirivimab, imdevimab, convalescent plasma, and interferons. Examples of suitable antiviral agents include, but are not limited to, baloxavir marboxil, oseltamivir, anamivir, vidarabine, acyclovir, ganciclovir, zidovudine, didanosine, zalcitabine, lamivudine, saquinavir, ritonavir, indinavir, nelfinavir, ribavirin, amantadine, rimantadine, remdesivir, favipiravir, and molnupiravir.

When the compounds (e.g., siNA molecules) described herein are co-administered with another, the effective amount may be less than when the compound is used alone.

### Examples

### Example 1: siNA Synthesis

This example describes an exemplary method for synthesizing ds-siNAs, such as the siNAs disclosed in Table 6 (as identified by the ds-siNA ID).

The 2′-OMe phosphoramidite 5′-O-DMT-deoxy Adenosine (NH-Bz), 3′-O-(2-cyanoethyl-N,N-diisopropyl phosphoramidite, 5′-O-DMT-deoxy Guanosine (NH-ibu), 3′-O-(2-cyanoethyl-N,N-diisopropyl phosphoramidite, 5′-O-DMT-deoxy Cytosine (NH-Bz), 3′-O-(2-cyanoethyl-N,N-diisopropyl phosphoramidite, 5′-O-DMT-Uridine 3′-O-(2-cyanoethyl-N,N-diisopropyl phosphoramidite and solid supports were purchased from Chemgenes Corp. MA.

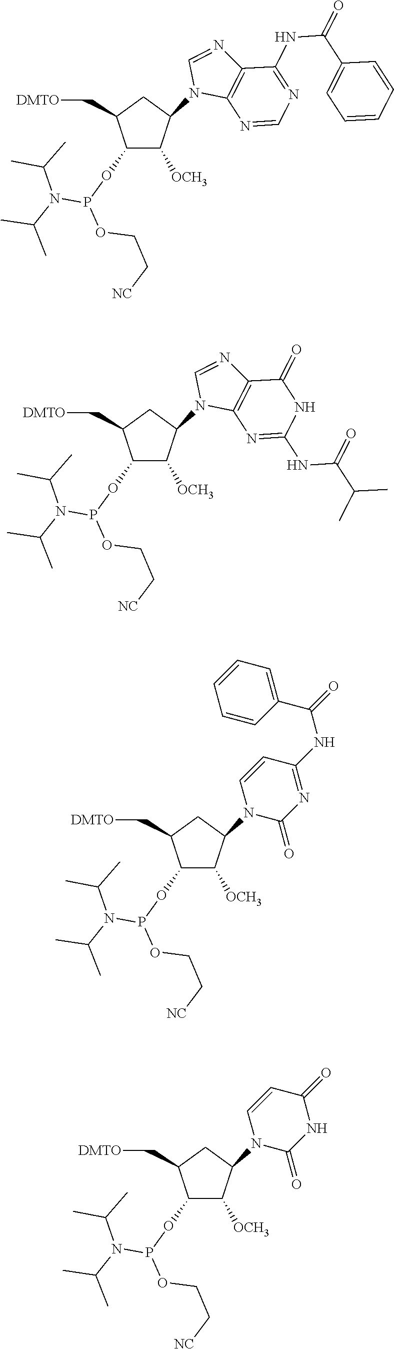

The 2′-F-5′-O-DMT-(NH-Bz) Adenosine-3′-O-(2-cyanoethyl-N,N-diisopropyl phosphoramidite, 2′-F-5′-O-DMT-(NH-ibu)-Guanosine, 3′-O-(2-cyanoethyl-N,N-diisopropyl phosphoramidite, 5′-O-DMT-(NH-Bz)-Cytosine, 2′-F-3′-O-(2-cyanoethyl-N,N-diisopropyl phosphoramidite, 5′-O-DMT-Uridine, 2′-F-3′-O-(2-cyanoethyl-N,N-diisopropyl phosphoramidite and solid supports were purchased from Thermo Fischer Milwaukee Wis., USA.

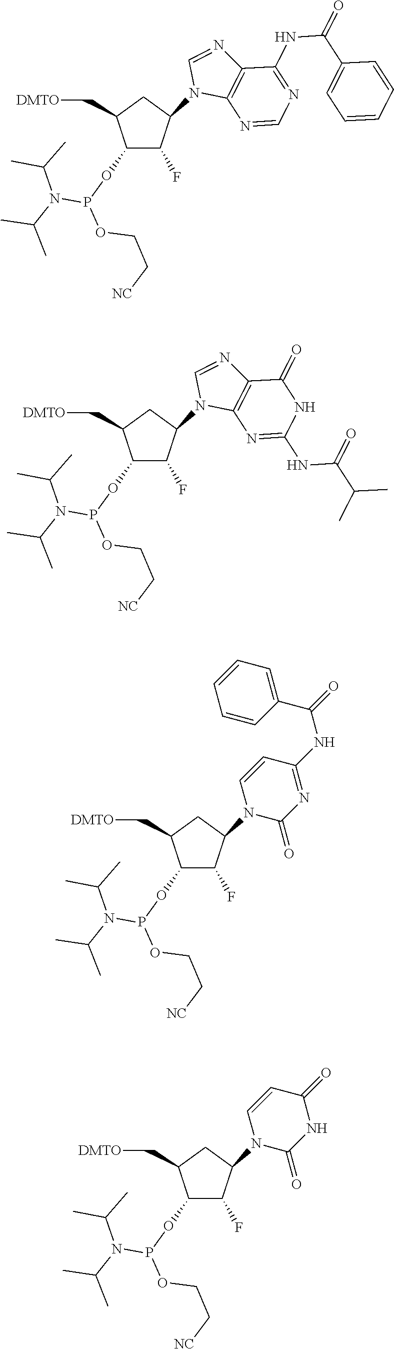

All the monomers were dried in vacuum desiccator with desiccants (P₂O₅, RT 24 h). The solid supports (CPG) attached to the nucleosides and universal supports was obtained from LGC and Chemgenes. The chemicals and solvents for post synthesis workflow were purchased from commercially available sources like VWR/Sigma and used without any purification or treatment. Solvent (Acetonitrile) and solutions (amidite and activator) were stored over molecular sieves during synthesis.

The oligonucleotides were synthesized on a DNA/RNA Synthesizers (Expedite 8909 or ABI-394) using standard oligonucleotide phosphoramidite chemistry starting from the 3′ residue of the oligonucleotide preloaded on CPG support. An extended coupling of 0.1M solution of phosphoramidite in CH₃CN in the presence of 5-(ethylthio)-1H-tetrazole activator to a solid bound oligonucleotide followed by standard capping, oxidation and deprotection afforded modified oligonucleotides. The 0.1M 12, THF:Pyridine; Water-7:2:1 was used as oxidizing agent while DDTT ((dimethylamino-methylidene) amino)-3H-1,2,4-dithiazaoline-3-thione was used as the sulfur-transfer agent for the synthesis of oligoribonucleotide phosphorothioates. The stepwise coupling efficiency of all modified phosphoramidites was more than 98%.

| Reagents | Detailed Description |
|---|---|
| Deblock Solution | 3% Dichloroacetic acid (DCA) in |
| | Dichloromethane (DCM) |
| Amidite Concentration | 0.1M in Anhydrous Acetonitrile |
| Activator | 0.25M Ethyl-thio-Tetrazole (ETT) |
| Cap-A solution | Acetic anhydride in Pyridine/THF |
| Cap-B Solution | 16% 1-Methylimidazole in THF |
| Oxidizing Solution | 0.02M I₂, THF:Pyridine; Water-7:2:1 |
| Sulfurizing Solution | 0.2M DDTT in Pyridine/Acetonitrile 1:1 |

Cleavage and Deprotection

Deprotection and cleavage from the solid support was achieved with mixture of ammonia methylamine (1:1, AMA) for 15 min at 65° C., when the universal linker was used, the deprotection was left for 90 min at 65° C. or solid supports were heated with aqueous ammonia (28%) solution at 55° C. for 16 h to deprotect the base labile protecting groups.

Quantitation of Crude SiNA or Raw Analysis

Samples were dissolved in deionized water (1.0 mL) and quantitated as follows: Blanking was first performed with water alone (2 ul) on Nanodrop then Oligo sample reading obtained at 260 nm. The crude material is dried down and stored at −20° C.

Crude HPLC/LC-MS analysis

The 0.1 OD of the crude samples were analyzed for crude HPLC and LC-MS analysis. After Confirming the crude LC-MS data then purification step was performed.

HPLC Purification

The unconjugated oligonucleotides were purified by anion-exchange HPLC. The buffers were 20 mM sodium phosphate in 10% CH₃CN, pH 8.5 (buffer A) and 20 mM sodium phosphate in 10% CH₃CN, 1.0 M NaBr, pH 8.5 (buffer B). Fractions containing full-length oligonucleotides were pooled.

Desalting of Purified SiNA

The purified dry siNA was then desalted using Sephadex G-25 M (Amersham Biosciences). The cartridge was conditioned with 10 mL of deionized water thrice. Finally, the purified siNA dissolved thoroughly in 2.5 mL RNAse free water was applied to the cartridge with very slow drop wise elution. The salt free siNA was eluted with 3.5 ml deionized water directly into a screw cap vial.

IEX HPLC and Electrospray LC/MS Analysis

Approximately 0.10 OD of siNA is dissolved in water and then pipetted in special vials for IEX-HPLC and LC/MS analysis. Analytical HPLC and ES LC-MS established the integrity of the compounds.

Duplex Preparation

Single strand oligonucleotides (Sense and Antisense strands) were annealed (1:1 by molar equivalents, heat 90° C. for 3 min followed by room temperature, 20 min) to give the duplex ds-siNA. The final compounds were analyzed on size exclusion chromatography (SEC).

### Example 2: Synthesis of 5′ End Cap Monomer

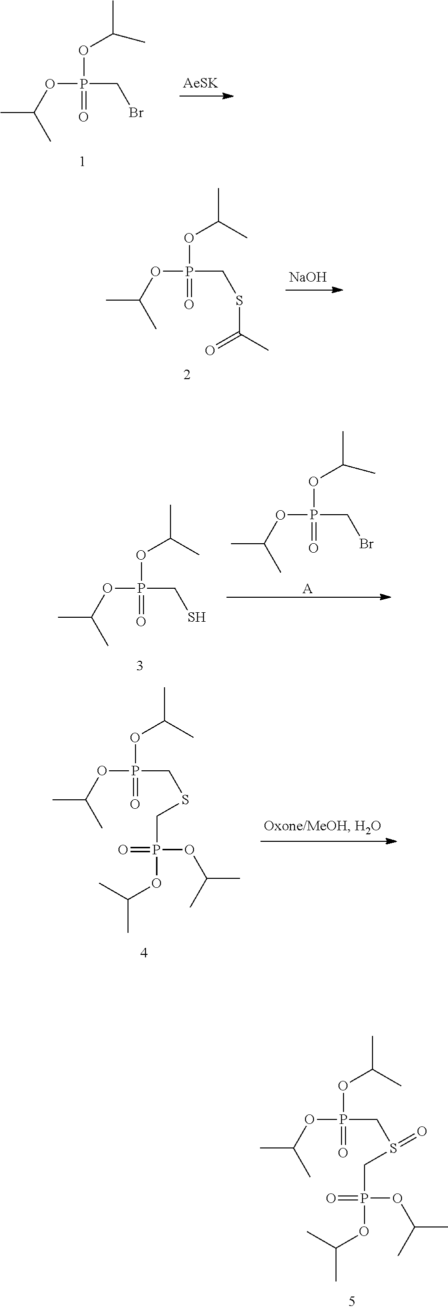

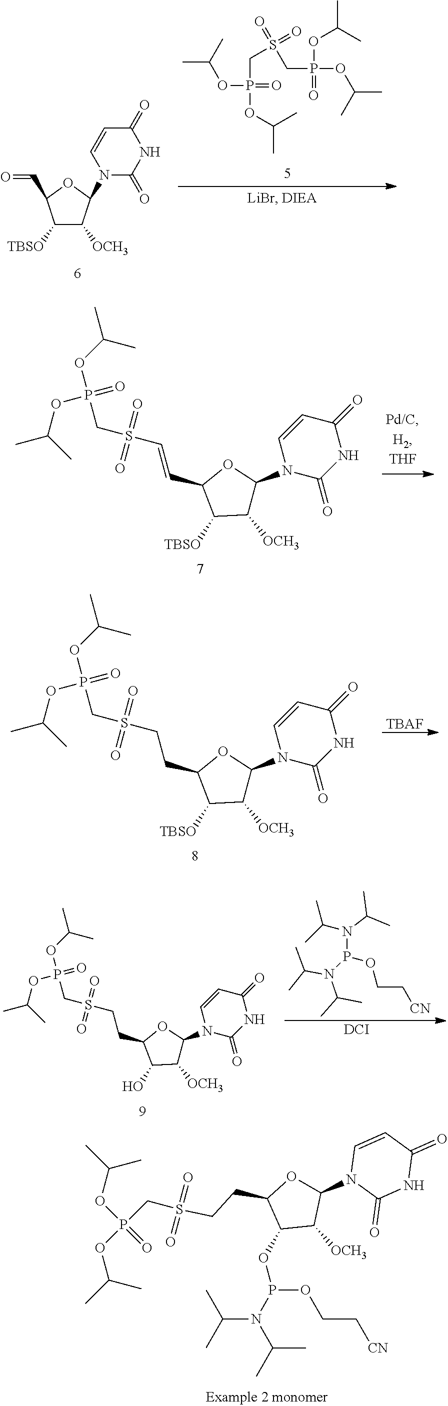

### Example 2 Monomer Synthesis Scheme

Preparation of (2): To a solution of 1 (15 g, 57.90 mmol) in DMF (150 mL) were added AcSK (11.24 g, 98.43 mmol) and TBAI (1.07 g, 2.89 mmol), and the mixture was stirred at 25° C. for 12 h. Upon completion as monitored by LCMS, the mixture was diluted with H₂O (10 mL) and extracted with EA (200 mL*3). The combined organic layers were washed with brine (200 mL*3), dried over anhydrous Na₂SO₄, filtered and concentrated under reduced pressure to give 2 (14.5 g, 96.52% yield, 98% purity) as a colorless oil. ESI-LCMS: 254.28 [M+H]⁺; ¹H NMR (400 MHz, CDCl₃) δ=4.78-4.65 (m, 2H), 3.19 (d, J=14.1 Hz, 2H), 2.38 (s, 3H), 1.32 (t, J=6.7 Hz, 12H); ³¹P NMR (162 MHz, CDCl₃) δ=20.59.

Preparation of (3): To a solution of 2 (14.5 g, 57.02 mmol) in CH₃CN (50 mL) and MeOH (25 mL) was added NaOH (3 M, 28.51 mL), and the mixture was stirred at 25° C. for 12 h under Ar. Upon completion as monitored by TLC, the reaction mixture was concentrated under reduced pressure to remove CH₃CN and CH₃OH. The residue was diluted with water (50 mL) and adjust pH=7 by 6M HCl, and the mixture was extracted with EA (50 mL*3). The combined organic layers were washed with brine (50 mL*3), dried over anhydrous Na₂SO₄, filtered and concentrated under reduced pressure to give 3 (12.1 g, crude) as a colorless oil.

Preparation of (4): To a solution of 3 (12.1 g, 57.01 mmol) in CH₃CN (25 mL) and MeOH (25 mL) was added A (14.77 g, 57.01 mmol) dropwise at 25° C., and the mixture was stirred at 25° C. under Ar for 12 h. Upon completion as monitored by LCMS, the reaction mixture was concentrated under reduced pressure to give 4 (19.5 g, 78.85% yield) as a colorless oil. ¹H NMR (400 MHz, CDCl₃) δ=4.80-4.66 (m, 4H), 2.93 (d, J=11.3 Hz, 4H), 1.31 (dd, J=3.9, 6.1 Hz, 24H); ³¹P NMR (162 MHz, CDCl₃) δ=22.18.

Preparation of (5): To a solution of 4 (19.5 g, 49.95 mmol) in MeOH (100 mL) and H₂O (100 mL) was added Oxone (61.41 g, 99.89 mmol) at 25° C. in portions, and the mixture was stirred at 25° C. for 12 h under Ar. Upon completion as monitored by LCMS, the reaction mixture was filtered, and the filtrate was concentrated under reduced pressure to remove MeOH. The residue was extracted with EA (50 mL*3). The combined organic layers were washed with brine (50 mL*3), dried over anhydrous Na₂SO₄, filtered and concentrated under reduced pressure to give a residue. The crude product was triturated with i-Pr₂O and n-Hexane (1:2, 100 mL) at 25° C. for 30 min to give 5 (15.6 g, 73.94% yield,) as a white solid. ¹H NMR (400 MHz, CDCl₃) δ=4.92-4.76 (m, 4H), 4.09 (d, J=16.1 Hz, 4H), 1.37 (dd, J=3.5, 6.3 Hz, 24H); ³¹P NMR (162 MHz, CDCl₃) δ=10.17.

Preparation of (7): To a mixture of 5 (6.84 g, 16.20 mmol) in THF (20 mL) was added LiBr (937.67 mg, 10.80 mmol) until dissolved, followed by DIEA (1.40 g, 10.80 mmol, 1.88 mL) under argon at 15° C. The mixture was stirred at 15° C. for 15 min. 6 (4 g, 10.80 mmol) were added. The mixture was stirred at 15° C. for 3 h. Upon completion as monitored by LCMS, the reaction mixture was quenched by addition of H₂O (40 mL) and extracted with EA (40 mL*3). The combined organic layers were washed with brine (100 mL), dried over Na₂SO₄, filtered and concentrated under reduced pressure to give a residue. The residue was purified by flash reverse-phase chromatography (120 g C-18 Column, Eluent of 0˜60% ACN/H₂O gradient @ 80 mL/min) to give 7 (5.7 g, 61.95% yield) as a colorless oil. ESI-LCMS: 611.2 [M+H]⁺, ¹H NMR (400 MHz, CDCl₃); δ=9.26 (s, 1H), 7.50 (d, J=8.1 Hz, 1H), 7.01 (s, 2H), 5.95 (d, J=2.7 Hz, 1H), 5.80 (dd, J=2.1, 8.2 Hz, 1H), 4.89-4.72 (m, 2H), 4.66 (d, J=7.2 Hz, 1H), 4.09-4.04 (m, 1H), 3.77 (dd, J=2.7, 4.9 Hz, 1H), 3.62 (d, J=3.1 Hz, 1H), 3.58 (d, J=3.1 Hz, 1H), 3.52 (s, 3H), 1.36 (td, J=1.7, 6.1 Hz, 12H), 0.92 (s, 9H), 0.12 (s, 6H); ³¹P NMR (162 MHz, CDCl₃) δ=9.02

Preparation of (8): To a mixture of 7 (5.4 g, 8.84 mmol) in THF (80 mL) was added Pd/C (5.4 g, 10% purity) under N₂. The suspension was degassed under vacuum and purged with H₂ several times. The mixture was stirred under H₂ (15 psi) at 20° C. for 1 hr. Upon completion as monitored by LCMS, the reaction mixture was filtered, and the filtrate was concentrated to give 8 (5.12 g, 94.5% yield) as a white solid. ESI-LCMS: 613.3 [M+H]⁺; H NMR (400 MHz, CD₃CN) δ=9.31 (s, 1H), 7.37 (d, J=8.0 Hz, 1H), 5.80-5.69 (m, 2H), 4.87-4.75 (m, 2H), 4.11-4.00 (m, 1H), 3.93-3.85 (m, 1H), 3.80-3.74 (m, 1H), 3.66-3.60 (m, 1H), 3.57-3.52 (m, 1H), 3.49 (s, 3H), 3.46-3.38 (m, 1H), 2.35-2.24 (m, 1H), 2.16-2.03 (m, 1H), 1.89-1.80 (m, 1H), 1.37-1.34 (m, 12H), 0.90 (s, 9H), 0.09 (s, 6H); ³¹P NMR (162 MHz, CD₃CN) δ=9.41.

Preparation of (9): To a solution of 8 (4.4 g, 7.18 mmol) in THF (7.2 mL) was added TBAF (1 M, 7.18 mL), and the mixture was stirred at 20° C. for 1 hr. Upon completion as monitored by LCMS, the reaction mixture was diluted with H₂O (50 mL) and extracted with EA (50 mL*4). The combined organic layers were washed with brine (50 mL), dried over Na₂SO₄, filtered and concentrated under reduced pressure to give a residue. The residue was purified by flash silica gel chromatography (ISCO®; 40 g SepaFlash® Silica Flash Column, Eluent of 0˜5%, MeOH/DCM gradient @ 40 mL/min) to give 9 (3.2 g, 88.50% yield) as a white solid. ESI-LCMS: 499.2 [M+H]⁺¹; ¹H NMR (400 MHz, CD₃CN) δ=9.21 (s, 1H), 7.36 (d, J=8.3 Hz, 1H), 5.81-5.72 (m, 2H), 4.88-4.74 (m, 2H), 3.99-3.87 (m, 2H), 3.84 (dd, 5.4 Hz, 1H), 3.66-3.47 (m, 7H), 2.98 (s, 1H), 2.44-2.15 (m, 2H), 1.36 (d, J=6.0 Hz, 12H); ³¹P NMR (162 MHz, CD₃CN) δ=9.48.

Preparation of (Example 2 monomer): To a mixture of 9 (3.4 g, 6.82 mmol, 1 eq) and 4 A MS (3.4 g) in MeCN (50 mL) was added P1 (2.67 g, 8.87 mmol, 2.82 mL, 1.3 eq) at 0° C., followed by addition of 1H-imidazole-4,5-dicarbonitrile (886.05 mg, 7.50 mmol) at 0° C. The mixture was stirred at 20° C. for 2 h. Upon completion as monitored by LCMS, the reaction mixture was quenched by addition of saturated aq. NaHCO₃ (50 mL) and diluted with DCM (100 mL). The organic layer was washed with saturated aq. NaHCO₃ (50 mL*2), dried over Na₂SO₄, filtered and concentrated under reduced pressure to give a residue. The residue was purified by prep-HPLC: column: YMC-Triart Prep C18 250*50 mm*10 um; mobile phase: [water (10 mM NH₄HCO₃)-ACN]; B %: 15% to give a impure product. The impure product was further purified by a flash silica gel column (0% to 5% i-PrOH in DCM with 0.5% TEA) to give Example 2 monomer (2.1 g, 43.18% yield) as a white solid. ESI-LCMS: 721.2 [M+Na]⁺; H NMR (400 MHz, CD₃CN) δ=9.29 (s, 1H), 7.45 (d, J=8.1 Hz, 1H), 5.81 (d, J=4.2 Hz, 1H), 5.65 (d, J=8.1 Hz, 1H), 4.79-4.67 (m, 2H), 4.26-4.05 (m, 2H), 4.00-3.94 (m, 1H), 3.89-3.63 (m, 6H), 3.53-3.33 (m, 5H), 2.77-2.61 (m, 2H), 2.31-2.21 (m, 1H), 2.16-2.07 (m, 1H), 1.33-1.28 (m, 12H), 1.22-1.16 (m, 1H), 1.22-1.16 (m, 11H); ³¹P NMR (162 MHz, CD₃CN) δ=149.89, 149.78, 10.07, 10.02.

### Example 3: Synthesis of 5′ End Cap Monomer

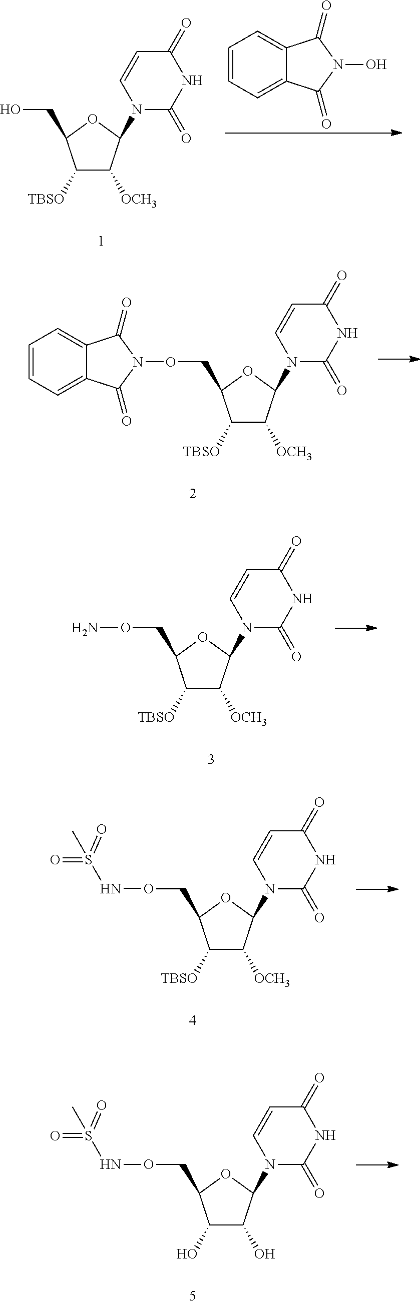

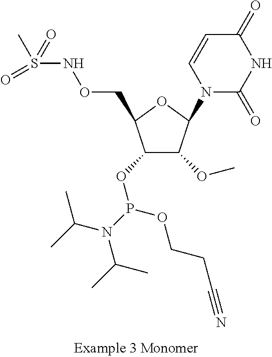 [SMILES: CO[C@@H]1[C@H](OP(OCCC#N)N(C(C)C)C(C)C)[C@@H](CONS(C)(=O)=O)O[C@H]1n1ccc(=O)[nH]c1=O]

### Example 3 Monomer Synthesis Scheme

Preparation of (2): To a solution of 1 (5 g, 13.42 mmol) in DMF (50 mL) were added PPh₃ (4.58 g, 17.45 mmol) and 2-hydroxyisoindoline-1,3-dione (2.85 g, 17.45 mmol), followed by a solution of DIAD (4.07 g, 20.13 mmol, 3.91 mL) in DMF (10 mL) dropwise at 15° C. The resulting solution was stirred at 15° C. for 18 hr. The reaction mixture was then diluted with DCM (50 mL), washed with H₂O (60 mL*3) and brine (30 mL), dried over Na₂SO₄, filtered and evaporated to give a residue. The residue was then triturated with EtOH (55 mL) for 30 min, and the collected white powder was washed with EtOH (10 mL*2) and dried to give 2 (12.2 g, 85.16% yield) as a white powder (the reaction was set up in two batches and combined) ESI-LCMS: 518.1 [M+H]⁺.

Preparation of (3): 2 (6 g, 11.59 mmol) was suspended in MeOH (50 mL), and then NH₂NH₂.H₂O (3.48 g, 34.74 mmol, 3.38 mL, 50% purity) was added dropwise at 20° C. The reaction mixture was stirred at 20° C. for 4 hr. Upon completion, the reaction mixture was diluted with EA (20 mL) and washed with NaHCO₃ (10 mL*2) and brine (10 mL). The combined organic layers were then dried over Na₂SO₄, filtered and evaporated to give 3 (8.3 g, 92.5% yield) as a white powder. (The reaction was set up in two batches and combined). ESI-LCMS: 388.0 [M+H]⁺, ¹H NMR (400 MHz, DMSO-d₆) δ=11.39 (br s, 1H), 7.72 (d, J=8.1 Hz, 1H), 6.24-6.09 (m, 2H), 5.80 (d, J=4.9 Hz, 1H), 5.67 (d, J=8.1 Hz, 1H), 4.26 (t, J=4.9 Hz, 1H), 4.03-3.89 (m, 1H), 3.87-3.66 (m, 3H), 3.33 (s, 3H), 0.88 (s, 9H), 0.09 (d, J=1.3 Hz, 6H)

Preparation of (4): To a solution of 3 (7 g, 18.06 mmol) and Py (1.43 g, 18.06 mmol, 1.46 mL) in DCM (130 mL) was added a solution of MSCl (2.48 g, 21.68 mmol, 1.68 mL) in DCM (50 mL) dropwise at −78° C. under N₂. The reaction mixture was allowed to warm to 15° C. in 30 min and stirred at 15° C. for 3 h. The reaction mixture was quenched by addition of ice-water (70 mL) at 0° C., and then extracted with DCM (50 mL*3). The combined organic layers were washed with saturated aq. NaHCO₃ (50 mL) and brine (30 mL), dried over Na₂SO₄, filtered and concentrated under reduced pressure to give a residue. The residue was purified by flash silica gel chromatography (ISCO®; 30 g SepaFlash® Silica Flash Column, Eluent of 0˜20% i-PrOH/DCM gradient @ 30 mL/min to give 4 (6.9 g, 77.94% yield) as a white solid. ESI-LCMS: 466.1 [M+H]⁺, ¹H NMR (400 MHz, DMSO-d₆) δ=11.41 (br s, 1H), 10.15 (s, 1H), 7.69 (d, J=8.1 Hz, 1H), 5.80 (d, J=4.4 Hz, 1H), 5.65 (d, J=8.1 Hz, 1H), 4.24 (t, J=5.2 Hz, 1H), 4.16-3.98 (m, 3H), 3.87 (t, J=4.8 Hz, 1H), 3.00 (s, 3H), 2.07 (s, 3H), 0.88 (s, 9H), 0.10 (d, J=1.5 Hz, 6H)

Preparation of (5): To a solution of 4 (6.9 g, 14.82 mmol) in THF (70 mL) was added TBAF (1 M, 16.30 mL) at 15° C. The reaction mixture was stirred at 15° C. for 18 hr, and then evaporated to give a residue. The residue was purified by flash silica gel chromatography (ISCO®; 24 g SepaFlash® Silica Flash Column, Eluent of 0˜9% MeOH/Ethyl acetate gradient @ 30 mL/min) to give 5 (1.8 g, 50.8% yield) as a white solid. ESI-LCMS: 352.0 [M+H]⁺; ¹H NMR (400 MHz, DMSO-d₆) δ=11.40 (s, 1H), 10.13 (s, 1H), 7.66 (d, J=8.1 Hz, 1H), 5.83 (d, J=4.9 Hz, 1H), 5.65 (dd, J=1.8, 8.1 Hz, 1H), 5.36 (d, J=6.2 Hz, 1H), 4.13-4.00 (m, 4H), 3.82 (t, J=5.1 Hz, 1H), 3.36 (s, 3H), 3.00 (s, 3H)

Preparation of (Example 3 monomer): To a mixture of 5 (3 g, 8.54 mmol) and DIEA (2.21 g, 17.08 mmol, 2.97 mL) in ACN (90 mL) was added P2 (3.03 g, 12.81 mmol) dropwise at 15° C. The reaction mixture was stirred at 15° C. for 5 h. Upon completion, the reaction mixture was diluted with EA (40 mL) and quenched with 5% NaHCO₃ (20 mL). The organic layer was washed with brine (30 mL), dried over Na₂SO₄, filtered and evaporated to give a residue. The residue was purified by flash silica gel chromatography (ISCO®; 12 g SepaFlash® Silica Flash Column, Eluent of 0˜15% i-PrOH/(DCM with 2% TEA) gradient @ 20 mL/min) to Example 3 monomer (2.1 g, 43.93% yield) as a white solid. ESI-LCMS: 552.3 [M+H]⁺; ¹H NMR (400 MHz, CD₃CN) δ=8.78 (br s, 1H), 7.57 (dd, J=4.6, 8.2 Hz, 1H), 5.97-5.80 (m, 1H), 5.67 (d, J=8.3 Hz, 1H), 4.46-4.11 (m, 4H), 3.95-3.58 (m, 5H), 3.44 (d, J=16.3 Hz, 3H), 3.02 (d, J=7.5 Hz, 3H), 2.73-2.59 (m, 2H), 1.23-1.15 (m, 12H); ³¹P NMR (162 MHz, CD₃CN) δ=150.30, 150.10

### Example 4: Synthesis of 5′ End Cap Monomer

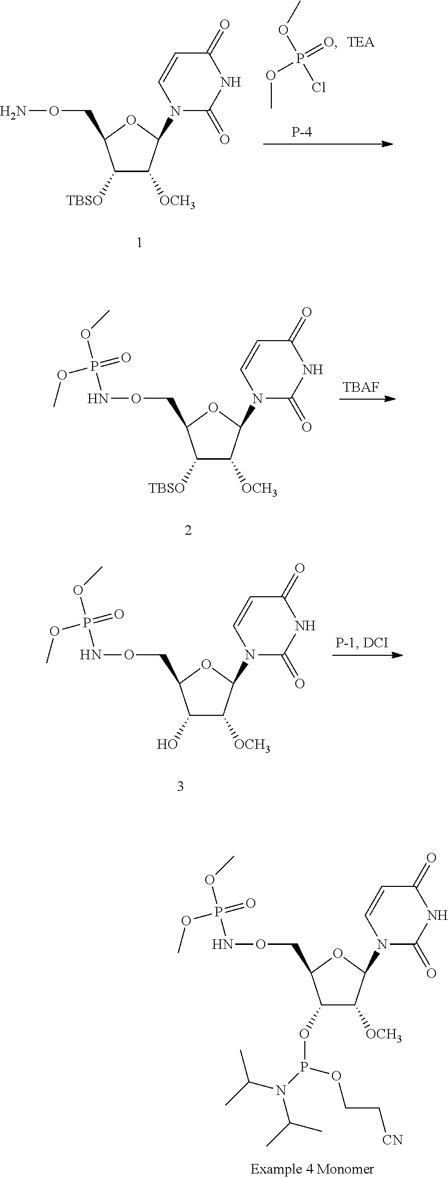

### Example 4 Monomer Synthesis Scheme

Preparation of (2): To the solution of 1 (5 g, 12.90 mmol) and TEA (1.57 g, 15.48 mmol, 2.16 mL) in DCM (50 mL) was added P-4 (2.24 g, 15.48 mmol, 1.67 mL) in DCM (10 mL) dropwise at 15° C. under N₂. The reaction mixture was stirred at 15° C. for 3 h. Upon completion as monitored by LCMS and TLC (PE:EtOAc=0:1), the reaction mixture was concentrated to dryness, diluted with H₂O (20 mL), and extracted with EA (50 mL*3). The combined organic layers were washed with brine (30 mL*3), dried over anhydrous Na₂SO₄, filtered, and the filtrate was concentrated under reduced pressure to give a residue. The residue was purified by flash silica gel chromatography (ISCO®; 40 g SepaFlash® Silica Flash Column, Eluent of 0˜95% Ethyl acetate/Petroleum ether gradient @ 60 mL/min) to give 2 (5.3 g, 71.3% yield) as a white solid. ESI-LCMS: 496.1 [M+H]⁺; H NMR (400 MHz, CDCl₃) δ=0.10 (d, J=4.02 Hz, 6H) 0.91 (s, 9H) 3.42-3.54 (m, 3H) 3.65-3.70 (m, 1H) 3.76-3.89 (m, 6H) 4.00 (dd, J=10.92, 2.89 Hz, 1H) 4.08-4.13 (m, 1H) 4.15-4.23 (m, 2H) 5.73 (dd, J=8.28, 2.01 Hz, 1H) 5.84 (d, J=2.76 Hz, 1H) 6.86 (d, J=15.81 Hz, 1H) 7.72 (d, J=8.03 Hz, 1H) 9.10 (s, 1H); ³¹P NMR (162 MHz, CD₃CN) δ=9.65

Preparation of (3): To a solution of 2 (8.3 g, 16.75 mmol) in THF (50 mL) were added TBAF (1 M, 16.75 mL) and CH₃COOH (1.01 g, 16.75 mmol, 957.95 uL). The mixture was stirred at 20° C. for 12 hr. Upon completion as monitored by LCMS, the reaction mixture was concentrated under reduced pressure. The residue was purified by column chromatography (SiO₂, PE: EA=0˜100%; MeOH/EA=0˜10%) to give 3 (5 g, 77.51% yield) as a white solid. ESI-LCMS: 382.1 [M+H]⁺; ¹H NMR (400 MHz, CDCl₃) δ=3.35 (s, 3H) 3.65 (br d, J=2.76 Hz, 3H) 3.68 (d, J=2.76 Hz, 3H) 3.77 (t, J=5.08 Hz, 1H) 3.84-4.10 (m, 4H) 5.33 (br d, J=5.52 Hz, 1H) 5.62 (d, J=7.77 Hz, 1H) 5.83 (d, J=4.94 Hz, 1H) 7.69 (d, J=7.71 Hz, 1H) 9.08 (d, J=16.81 Hz, 1H) 11.39 (br s, 1H); ³¹P NMR (162 MHz, CD₃CN) δ=15.41

Preparation of (Example 4 monomer): To a solution of 3 (2 g, 5.25 mmol) and DIPEA (2.03 g, 15.74 mmol, 2.74 mL, 3 eq) in MeCN (21 mL) and pyridine (7 mL) was added P2 (1.86 g, 7.87 mmol) dropwise at 20° C., and the mixture was stirred at 20° C. for 3 hr. Upon completion as monitored by LCMS, the reaction mixture was diluted with water (20 mL) and extracted with EA (50 mL). The combined organic layers were washed with brine (30 mL), dried over anhydrous Na₂SO₄, filtered, and the filtrate was concentrated under reduced pressure to give a residue. The residue was purified by flash silica gel chromatography (ISCO®; 25 g SepaFlash® Silica Flash Column, Eluent of 0˜45% (Ethyl acetate:EtOH=4:1)/Petroleum ether gradient) to give Example 4 monomer (1.2 g, 38.2% yield) as a white solid. ESI-LCMS: 604.1 [M+H]⁺; ¹H NMR (400 MHz, CD₃CN) δ=1.12-1.24 (m, 12H) 2.61-2.77 (m, 2H) 3.43 (d, J=17.64 Hz, 3H) 3.59-3.69 (m, 2H) 3.71-3.78 (m, 6H) 3.79-4.14 (m, 5H) 4.16-4.28 (m, 1H) 4.29-4.42 (m, 1H) 5.59-5.72 (m, 1H) 5.89 (t, J=4.53 Hz, 1H) 7.48 (br d, J=12.76 Hz, 1H) 7.62-7.74 (m, 1H) 9.26 (br s, 1H); ³¹P NMR (162 MHz, CD₃CN) δ=150.57, 149.96, 9.87

### Example 5: Synthesis of 5′ End Cap Monomer

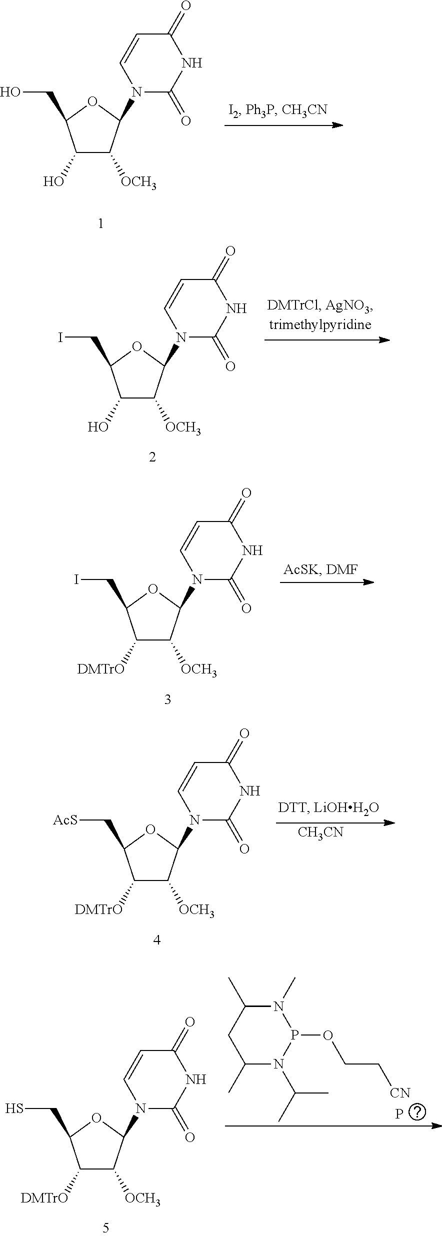

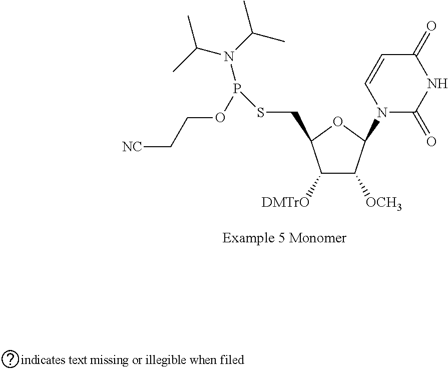

### Example 5 Monomer Synthesis Scheme

Preparation of (2): To a solution of 1 (30 g, 101.07 mmol, 87% purity) in CH₃CN (1.2 L) and Py (60 mL) were added 12 (33.35 g, 131.40 mmol, 26.47 mL) and PPh₃ (37.11 g, 141.50 mmol) in one portion at 10° C. The reaction was stirred at 25° C. for 48 h. Upon completion, the mixture was diluted with saturated aq.Na₂S₂O₃ (300 mL) and saturated aq.NaHCO₃ (300 mL), concentrated to remove CH₃CN, and extracted with EtOAc (300 mL*3). The combined organic layers were washed with brine (300 mL), dried over Na₂SO₄, filtered and concentrated under reduced pressure to give a residue. The residue was purified by flash silica gel chromatography (ISCO®; 330 g SepaFlash® Silica Flash Column, Eluent of 0˜60% Methanol/Dichloromethane gradient @ 100 mL/min) to give 2 (28.2 g, 72% yield) as a brown solid. ESI-LCMS: 369.1 [M+H]⁺; H NMR (400 MHz, DMSO-d₆) δ=11.43 (s, 1H), 7.68 (d, J=8.1 Hz, 1H), 5.86 (d, J=5.5 Hz, 1H), 5.69 (d, J=8.1 Hz, 1H), 5.46 (d, J=6.0 Hz, 1H), 4.08-3.96 (m, 2H), 3.90-3.81 (m, 1H), 3.60-3.51 (m, 1H), 3.40 (dd, J=6.9, 10.6 Hz, 1H), 3.34 (s, 3H).

Preparation of (3): To the solution of 2 (12 g, 32.6 mmol) in DCM (150 mL) were added AgNO₃ (11.07 g, 65.20 mmol), 2,4,6-trimethylpyridine (11.85 g, 97.79 mmol, 12.92 mL), and DMTCl (22.09 g, 65.20 mmol) at 10° C., and the reaction mixture was stirred at 10° C. for 16 hr. Upon completion, the mixture was filtered and the filtrate was concentrated under reduced pressure. The residue was purified by flash silica gel chromatography (ISCO®; 120 g SepaFlash® Silica Flash Column, Eluent of 0˜50% Ethyl acetate/Petroleum ether gradient @ 60 mL/min) to give 3 (17 g, 70.78% yield) as a yellow solid. ESI-LCMS: 693.1 [M+Na]⁺¹; H NMR (400 MHz, DMSO-d₆) δ=11.46 (s, 1H), 7.60 (d, J=8.4 Hz, 1H), 7.49 (d, J=7.2 Hz, 2H), 7.40-7.30 (m, 6H), 7.29-7.23 (m, 1H), 6.93 (d, J=8.8 Hz, 4H), 5.97 (d, J=6.0 Hz, 1H), 5.69 (d, J=8.0 Hz, 1H), 4.05-4.02 (m, 1H), 3.75 (d, J=1.2 Hz, 6H), 3.57 (t, J=5.6 Hz, 1H), 3.27 (s, 4H), 3.06 (t, J=10.4 Hz, 1H), 2.98-2.89 (m, 1H).

Preparation of (4): To a solution of 3 (17 g, 25.35 mmol) in DMF (200 mL) was added AcSK (11.58 g, 101.42 mmol) at 25° C., and the reaction was stirred at 60° C. for 2 hr. The mixture was diluted with H₂O (600 mL) and extracted with EtOAc (300 mL*4). The combined organic layers were washed with brine (300 mL), dried over Na₂SO₄, filtered, and concentrated under reduced pressure to give 4 (15.6 g, crude) as a brown solid, which was used directly without further purification. ESI-LCMS: 641.3 [M+H]⁺.

Preparation of (5): To a solution of 4 (15.6 g, 25.21 mmol) in CH₃CN (200 mL) were added DTT (11.67 g, 75.64 mmol, 11.22 mL) and LiOH.H₂O (1.06 g, 25.21 mmol) at 10° C. under Ar. The reaction was stirred at 10° C. for 1 hr. The mixture was concentrated under reduced pressure to remove CH₃CN, and the residue was diluted with H₂O (400 mL) and extracted with EtOAc (200 mL*3). The combined organic layers were washed with brine (300 mL), dried over Na₂SO₄, filtered and concentrated under reduced pressure to give a residue. The residue was purified by flash silica gel chromatography (ISCO®; 220 g SepaFlash® Silica Flash Column, Eluent of 0˜60% Ethyl acetate/Petroleum ether gradient @ 100 mL/min) to give 5 (8.6 g, 56.78% yield) as a white solid. ESI-LCMS: 599.3 [M+Na]⁺; ¹H NMR (400 MHz, DMSO-d₆) δ=8.79 (s, 1H), 7.61 (d, J=8.0 Hz, 1H), 7.56-7.46 (m, 2H), 7.45-7.37 (m, 4H), 7.36-7.27 (m, 3H), 6.85 (dd, J=2.8, 8.8 Hz, 4H), 5.85 (d, J=1.3 Hz, 1H), 5.68 (dd, J=2.0, 8.2 Hz, 1H), 4.33-4.29 (m, 1H), 3.91 (dd, J=4.8, 8.2 Hz, 1H), 3.81 (d, J=1.6 Hz, 6H), 3.33 (s, 3H), 2.85-2.80 (m, 1H), 2.67-2.55 (m, 2H), 1.11 (t, J=8.8 Hz, 1H).

Preparation of (Example 5 monomer): To a solution of 5 (6 g, 10.40 mmol) in DCM (120 mL) were added P1 (4.08 g, 13.53 mmol, 4.30 mL) and DCI (1.35 g, 11.45 mmol) in one portion at 10° C. under Ar. The reaction was stirred at 10° C. for 2 hr. The reaction mixture was diluted with saturated aq.NaHCO₃ (50 mL) and extracted with DCM (20 mL*3). The combined organic layers were washed with brine (30 mL), dried over Na₂SO₄, filtered and concentrated under reduced pressure to give a residue. The residue was purified by prep-HPLC (column: YMC-Triart Prep C18 250*50 mm*10 um; mobile phase: [water (10 mM NH₄HCO₃)-ACN]; B %: 35%-81%, 20 min) to give Example 5 monomer (3.54 g, 43.36% yield) as a yellow solid. ESI-LCMS: 776.4 [M+H]⁺; ¹H NMR (400 MHz, DMSO-d₆) δ=7.65-7.38 (m, 7H), 7.37-7.22 (m, 3H), 6.90 (d, J=8.4 Hz, 4H), 5.92 (s, 1H), 5.66 (t, J=8.2 Hz, 1H), 4.13 (d, J=4.0 Hz, 1H), 4.00-3.88 (m, 1H), 3.87-3.59 (m, 10H), 3.33 (d, J=5.8 Hz, 3H), 3.12-2.94 (m, 1H), 2.78-2.60 (m, 3H), 2.55-2.48 (m, 1H), 1.36-0.98 (m, 12H); ³¹P NMR (162 MHz, DMSO-d₆) δ=162.69.

### Example 6: Synthesis of 5′ End Cap Monomer

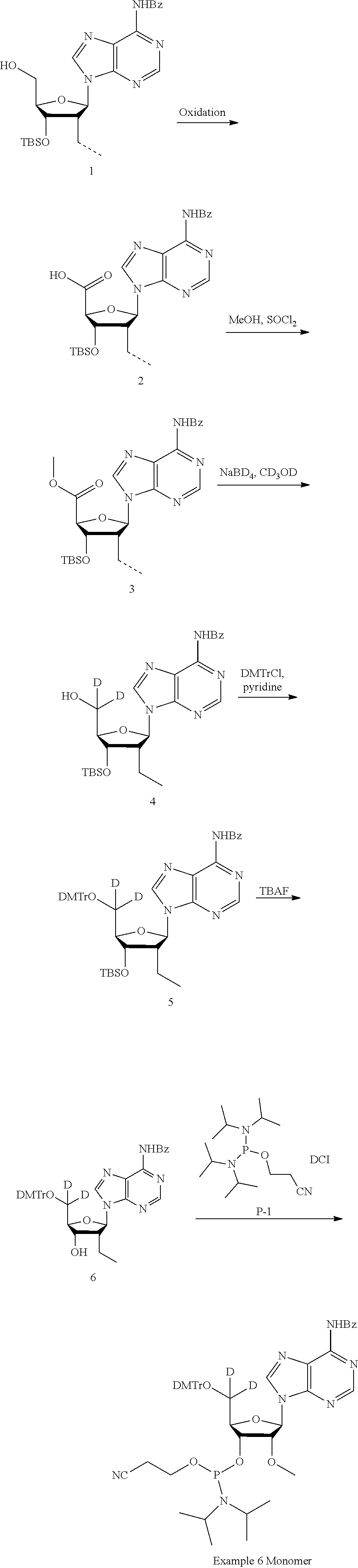

### Example 6 Monomer Synthesis Scheme

Preparation of (2): To a solution of 1 (22.6 g, 45.23 mmol) in DCM (500 mL) and H₂O (125 mL) were added TEMPO (6.40 g, 40.71 mmol) and DIB (29.14 g, 90.47 mmol) at 0° C. The mixture was stirred at 20° C. for 20 h. Upon completion as monitored by LCMS, saturated aq. NaHCO₃ was added to the mixture to adjust pH >8. The mixture was diluted with H₂O (200 mL) and washed with DCM (100 mL*3). The aqueous layer was collected, adjusted to pH<5 by HCl (4M), and extracted with DCM (200 mL*3). The combined organic layers were washed with brine (300 mL), dried over Na₂SO₄, filtered, and concentrated under reduced pressure to give 2 (17.5 g, 68.55% yield) as a yellow solid. ESI-LCMS: 514.2 [M+H]⁺; ¹H NMR (400 MHz, DMSO-d₆) δ=11.27 (s, 1H), 8.86 (s, 1H), 8.78 (s, 1H), 8.06 (d, J=7.5 Hz, 2H), 7.68-7.62 (m, 1H), 7.59-7.52 (m, 2H), 6.28 (d, J=6.8 Hz, 1H), 4.82-4.76 (m, 1H), 4.54 (dd, J=4.1, 6.7 Hz, 1H), 4.48 (d, J=1.8 Hz, 1H), 3.32 (s, 3H), 0.94 (s, 9H), 0.18 (d, J=4.8 Hz, 6H).

Preparation of (3): To a solution of 2 (9.3 g, 18.11 mmol) in MeOH (20 mL) was added SOCl₂ (3.23 g, 27.16 mmol, 1.97 mL) dropwise at 0° C. The mixture was stirred at 20° C. for 0.5 hr. Upon completion as monitored by LCMS, the reaction mixture was quenched by addition of saturated aq. NaHCO₃ (80 mL) and concentrated under reduced pressure to remove MeOH. The aqueous layer was extracted with DCM (80 mL*3). The combined organic layers were washed with brine (200 mL), dried over Na₂SO₄, filtered and concentrated under reduced pressure to give a residue. The residue was purified by flash silica gel chromatography (ISCO®; 120 g SepaFlash® Silica Flash Column, Eluent of 0˜5%, MeOH/DCM gradient @ 85 mL/min) to give 3 (5.8 g, 60% yield) as a yellow solid. ESI-LCMS: 528.3 [M+H]⁺, ¹H NMR (400 MHz, DMSO-d₆) δ=11.28 (s, 1H), 8.79 (d, J=7.3 Hz, 2H), 8.06 (d, J=7.5 Hz, 2H), 7.68-7.62 (m, 1H), 7.60-7.53 (m, 2H), 6.28 (d, J=6.6 Hz, 1H), 4.87 (dd, J=2.4, 4.0 Hz, 1H), 4.61 (dd, J=4.3, 6.5 Hz, 1H), 4.57 (d, J=2.2 Hz, 1H), 3.75 (s, 3H), 3.32 (s, 3H), 0.94 (s, 9H), 0.17 (d, J=2.2 Hz, 6H).

Preparation of (4): To a mixture of 3 (5.7 g, 10.80 mmol) in CD₃OD (120 mL) was added NaBD₄ (1.63 g, 43.21 mmol) in portions at 0° C., and the mixture was stirred at 20° C. for 1 hr. Upon completion as monitored by LCMS, the reaction mixture was neutralized by AcOH (˜10 mL) and concentrated under reduced pressure to give a residue. The residue was purified by flash silica gel chromatography (ISCO®; 40 g SepaFlash® Silica Flash Column, Eluent of 0˜5%, MeOH/DCM gradient @ 40 mL/min) to give 4 (4.15 g, 7.61 mmol, 70.45% yield) as a yellow solid. ESI-LCMS: 502.2 [M+H]⁺; ¹H NMR (400 MHz, DMSO-d₆) δ=11.23 (s, 1H), 8.76 (s, 2H), 8.04 (d, J=7.3 Hz, 2H), 7.69-7.62 (m, 1H), 7.60-7.52 (m, 2H), 6.14 (d, J=6.0 Hz, 1H), 5.18 (s, 1H), 4.60-4.51 (m, 2H), 3.98 (d, J=3.0 Hz, 1H), 3.32 (s, 3H), 0.92 (s, 9H), 0.13 (d, J=1.5 Hz, 6H).

Preparation of (5): To a solution of 4 (4.85 g, 9.67 mmol) in pyridine (50 mL) was added DMTrCl (5.90 g, 17.40 mmol) at 25° C. and the mixture was stirred for 2 hr. Upon completion as monitored by LCMS, the reaction mixture was concentrated under reduced pressure to remove pyridine. The residue was diluted with EtOAc (150 mL) and washed with H₂O (50 mL*3), dried over Na₂SO₄, filtered and concentrated under reduced pressure to give a residue. The residue was purified by flash silica gel chromatography (ISCO®; 80 g SepaFlash® Silica Flash Column, Eluent of 0˜70%, EA/PE gradient @ 60 mL/min) to give 5 (6.6 g, 84.06% yield) as a yellow solid. ESI-LCMS: 804.3[M+H]⁺, ¹H NMR (400 MHz, DMSO-d₆) δ=11.22 (s, 1H), 8.68 (d, J=11.0 Hz, 2H), 8.03 (d, J=7.3 Hz, 2H), 7.68-7.60 (m, 1H), 7.58-7.49 (m, 2H), 7.37-7.30 (m, 2H), 7.27-7.16 (m, 7H), 6.88-6.79 (m, 4H), 6.17 (d, J=4.2 Hz, 1H), 4.72 (t, J=5.0 Hz, 1H), 4.60 (t, J=4.5 Hz, 1H), 4.03-3.98 (m, 1H), 3.71 (s, 6H), 0.83 (s, 9H), 0.12-0.03 (m, 6H).

Preparation of (6): To a solution of 5 (6.6 g, 8.21 mmol) in THF (16 mL) was added TBAF (1 M, 8.21 mL,), and the mixture was stirred at 20° C. for 2 hr. Upon completion as monitored by LCMS, the reaction mixture was diluted with EA (150 mL) and washed with H₂O (50 mL*3). The organic layer was washed with brine (150 mL), dried over Na₂SO₄, filtered and concentrated under reduced pressure to give a residue. The residue was purified by flash silica gel chromatography (ISCO®; 80 g SepaFlash® Silica Flash Column, Eluent of 10-100%, EA/PE gradient @ 30 mL/min) to give 6 (5.4 g, 94.4% yield) as a yellow solid. ESI-LCMS: 690.3 [M+H]⁺; ¹H NMR (400 MHz, DMSO-d₆) δ=11.24 (s, 1H), 8.69 (s, 1H), 8.62 (s, 1H), 8.05 (d, J=7.3 Hz, 2H), 7.69-7.62 (m, 1H), 7.60-7.52 (m, 2H), 7.40-7.33 (m, 2H), 7.30-7.18 (m, 7H), 6.84 (dd, J=5.9, 8.9 Hz, 4H), 6.19 (d, J=4.8 Hz, 1H), 5.36 (d, J=6.0 Hz, 1H), 4.59-4.52 (m, 1H), 4.48 (q, J=5.1 Hz, 1H), 4.11 (d, J=4.8 Hz, 1H), 3.72 (d, J=1.0 Hz, 6H), 3.40 (s, 3H).

Preparation of (Example 6 monomer): To a solution of 6 (8.0 g, 11.60 mmol) in MeCN (150 mL) was added P-1 (4.54 g, 15.08 mmol, 4.79 mL) at 0° C., followed by DCI (1.51 g, 12.76 mmol) in one portion. The mixture was warmed to 20° C. and stirred for 2 h. Upon completion as monitored by LCMS, the reaction mixture was quenched by addition of saturated aq. NaHCO₃ (50 mL) and diluted with DCM (250 mL). The organic layer was washed with saturated aq.NaHCO₃ (50 mL*2), dried over Na₂SO₄, filtered and concentrated under reduced pressure. The residue was purified by a flash silica gel column (0% to 60% EA in PE contain 0.5% TEA) to give Example 6 monomer (5.75 g, 55.37% yield, 99.4% purity) as a white solid. ESI-LCMS: 890.4 [M+H]⁺; ¹H NMR (400 MHz, CD₃CN) δ=9.55 (s, 1H), 8.63-8.51 (m, 1H), 8.34-8.24 (m, 1H), 7.98 (br d, J=7.5 Hz, 2H), 7.65-7.55 (m, 1H), 7.53-7.46 (m, 2H), 7.44-7.37 (m, 2H), 7.32-7.17 (m, 7H), 6.84-6.77 (m, 4H), 6.14 (d, J=4.3 Hz, 1H), 4.84-4.73 (m, 1H), 4.72-4.65 (m, 1H), 4.34-4.27 (m, 1H), 3.91-3.61 (m, 9H), 3.50-3.43 (m, 3H), 2.72-2.61 (m, 1H), 2.50 (t, J=6.0 Hz, 1H), 1.21-1.15 (m, 10H), 1.09 (d, J=6.8 Hz, 2H); ³¹P NMR (162 MHz, CD₃CN) δ =150.01, 149.65

### Example 7: Synthesis of 5′ End Cap Monomer

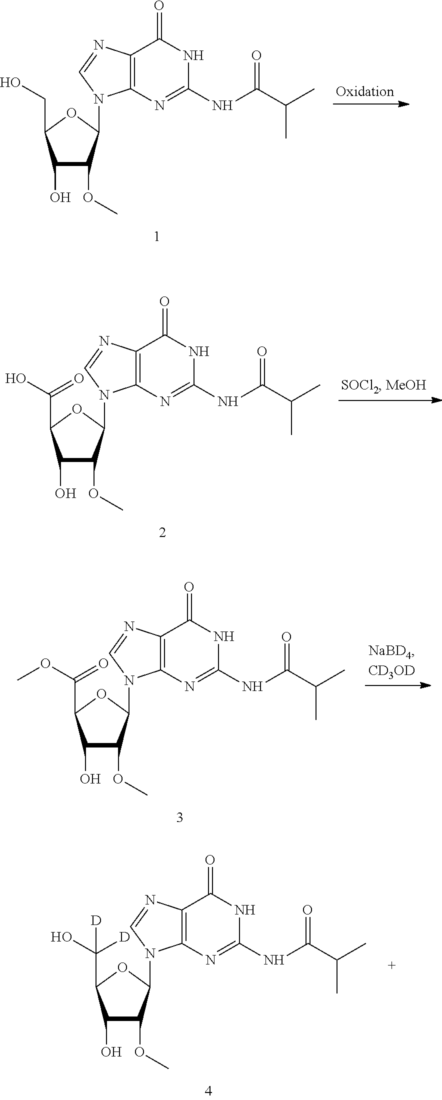

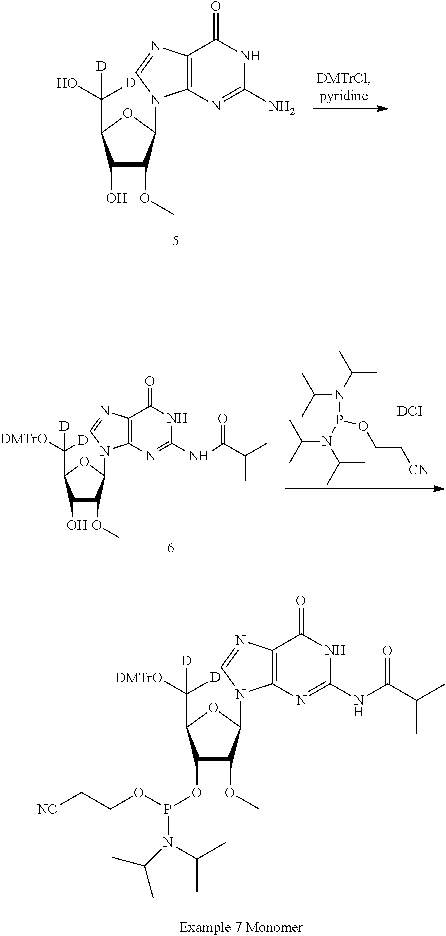

### Example 7 Monomer Synthesis Scheme

Preparation of (2): To a solution of 1 (10 g, 27.22 mmol) in CH₃CN (200 mL) and H₂O (50 mL) were added TEMPO (3.85 g, 24.50 mmol) and DIB (17.54 g, 54.44 mmol). The mixture was stirred at 25° C. for 12 h. Upon completion as monitored by LCMS, the reaction mixture was concentrated under reduced pressure to give a residue. The residue was triturated with EtOAc (600 mL) for 30 min. The resulting suspension was filtered and the collected solid was washed with EtOAc (300 mL*2) to give 2 (20.09 g, 91.5% yield) as a white solid. ESI-LCMS: 382.0 [M+H]⁺.

Preparation of (3): To a solution of 2 (6 g, 15.73 mmol) in MeOH (100 mL) was added SOCl₂ (2.81 g, 23.60 mmol, 1.71 mL) dropwise at 0° C. The mixture was stirred at 25° C. for 12 h. Upon completion as monitored by LCMS, the reaction mixture was quenched by addition of NaHCO₃ (4 g) and stirred at 25° C. for 30 min. The reaction mixture was filtered and the filtrate was concentrated under reduced pressure to give 3 (18.8 g, 95.6% yield) as a white solid. The crude product was used for the next step without further purification. (The reaction was set up in parallel 3 batches and combined). ESI-LCMS: 396.1 [M+H]⁺; ¹H NMR (400 MHz, DMSO-d₆) δ=12.26-11.57 (m, 2H), 8.42-8.06 (m, 1H), 6.14-5.68 (m, 2H), 4.56 (s, 2H), 4.33 (dd, J=4.0, 7.3 Hz, 1H), 3.77 (m, 3H), 3.30 (s, 3H), 2.81-2.69 (m, 1H), 1.11 (s, 6H)

Preparation of (4 & 5): To a mixture of 3 (10.1 g, 25.55 mmol) in CD₃OD (120 mL) was added NaBD₄ (3.29 g, 86.86 mmol, 3.4 eq) in portions at 0° C. The mixture was stirred at 25° C. for 1 h. Upon completion as monitored by LCMS, the reaction mixture was neutralized with AcOH (˜15 mL) and concentrated under reduced pressure to give a residue. The residue was purified by flash silica gel chromatography (ISCO®; 120 g SepaFlash® Silica Flash Column, Eluent of 0˜7.4%, MeOH/DCM gradient @ 80 mL/min) to give 4 (2.98 g, 6.88 mmol, 27% yield) as a yellow solid. ESI-LCMS: 370.1[M+H]⁺ and 5 (10.9 g, crude) as a yellow solid. ESI-LCMS: 300.1[M+H]⁺; ¹H NMR (400 MHz, CD₃OD) δ=7.85 (s, 1H), 5.87 (d, J=6.0 Hz, 1H), 4.46-4.39 (m, 1H), 4.34 (t, J=5.4 Hz, 1H), 4.08 (d, J=3.1 Hz, 1H), 3.49-3.38 (m, 4H)

Preparation of 6: To a solution of 4 (1.9 g, 4.58 mmol, 85.7% purity) in pyridine (19 mL) was added DMTrCl (2.02 g, 5.96 mmol). The mixture was stirred at 25° C. for 2 h under N₂. Upon completion as monitored by LCMS, the reaction mixture was quenched by MeOH (10 mL) and concentrated under reduce pressure to give a residue. The residue was diluted with H₂O (10 mL*3) and extracted with EA (20 mL*3). The combined organic layers were washed with brine (20 mL), dried over anhydrous Na₂SO₄, filtered and concentrated under reduce pressure to give a residue. The residue was purified by flash silica gel chromatography (ISCO®; 25 g SepaFlash® Silica Flash Column, Eluent of 0˜77%, PE: (EA with 10% EtOH): 1% TEA@ 35 mL/min) to give 6 (2.6 g, 81.71% yield, 96.71% purity) as a white foam. ESI-LCMS: 672.2 [M+H]⁺; ¹H NMR (400 MHz, CDCl₃) δ=12.02 (s, 1H), 7.96 (s, 1H), 7.83 (s, 1H), 7.51 (d, J=7.4 Hz, 2H), 7.37 (d, J=8.6 Hz, 4H), 7.25-7.17 (m, 2H), 6.80 (t, J=8.4 Hz, 4H), 5.88 (d, J=6.3 Hz, 1H), 4.69 (t, J=5.7 Hz, 1H), 4.64 (s, 1H), 4.54 (s, 1H), 4.19 (d, J=2.9 Hz, 1H), 3.77 (d, J=4.5 Hz, 6H), 3.60-3.38 (m, 3H), 2.81 (s, 1H), 1.81 (td, J=6.9, 13.7 Hz, 1H), 0.97 (d, J=6.8 Hz, 3H), 0.80 (d, J=6.9 Hz, 3H)

Preparation of Example 7 monomer: To a solution of 6 (8.4 g, 12.5 mmol) in MeCN (80 mL) was added P-1 (4.9 g, 16.26 mmol, 5.16 mL) at 0° C., followed by addition of DCI (1.624 g, 13.76 mmol) in one portion at 0° C. under Ar. The mixture was stirred at 25° C. for 2 h. Upon completion as monitored by LCMS, the reaction mixture was quenched with saturated aq.NaHCO₃ (20 mL) and extracted with DCM (50 mL*2). The combined organic layers were dried over anhydrous Na₂SO₄, filtered and concentrated under reduce pressure to give a residue. The residue was purified by flash silica gel chromatography (ISCO®; 40 g SepaFlash® Silica Flash Column, Eluent of 0˜52% PE: EA (10% EtOH): 5% TEA, @ 80 mL/min) to give Example 7 monomer (3.4 g, 72.1% yield,) as a white foam. ESI-LCMS: 872.4 [M+H]⁺; ¹H NMR (400 MHz, CD₃CN) δ=12.46-11.07 (m, 1H), 9.29 (s, 1H), 7.84 (d, J=14.6 Hz, 1H), 7.42 (t, J=6.9 Hz, 2H), 7.34-7.17 (m, 7H), 6.85-6.77 (m, 4H), 5.95-5.77 (m, 1H), 4.56-4.40 (m, 2H), 4.24 (dd, J=4.0, 13.3 Hz, 1H), 3.72 (d, J=2.0 Hz, 7H), 3.66-3.53 (m, 3H), 3.42 (d, J=11.8 Hz, 3H), 2.69-2.61 (m, 1H), 2.60-2.42 (m, 2H), 1.16-1.00 (m, 18H); ³¹P NMR (162 MHz, CD₃CN) δ=149.975, 149.9

### Example 8: Synthesis of 5′ End Cap Monomer

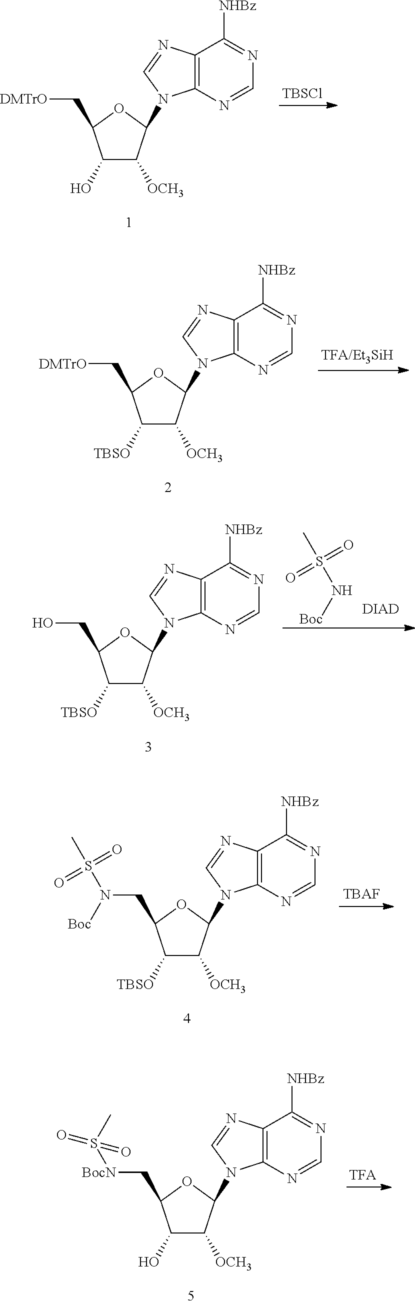

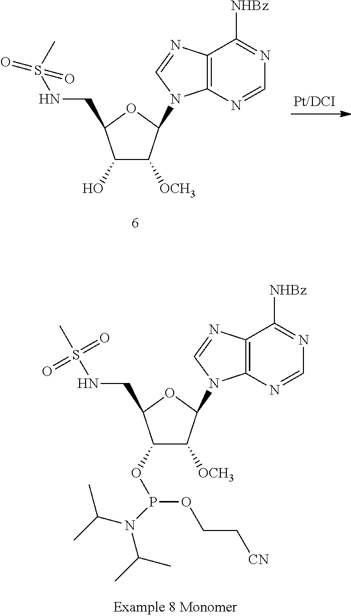

### Example 8 Monomer Synthesis Scheme

Preparation of (2): To a solution of 1 (40 g, 58.16 mmol) in DMF (60 mL) were added imidazole (11.88 g, 174.48 mmol), NaI (13.08 g, 87.24 mmol), and TBSCl (17.52 g, 116.32 mmol) at 20° C. in one portion. The reaction mixture was stirred at 20° C. for 12 h. Upon completion, the mixture was diluted with EA (200 mL). The organic layer was washed with brine/water (80 mL/80 mL*4), dried over Na₂SO₄, filtered and evaporated to give 2 (50.8 g, crude) as yellow solid. ESI-LCMS: 802.3 [M+H]⁺

Preparation of (3): To a solution of 2 (8.4 g, 10.47 mmol) in DCM (120 mL) were added Et₃SiH (3.06 g, 26.3 mmol, 4.2 mL) and TFA (1.29 g, 0.84 mL) dropwise at 0° C. The reaction mixture was stirred at 20° C. for 2 h. The reaction mixture was washed with saturated aq.NaHCO₃ (15 mL) and brine (80 mL). The organic layer was dried over Na₂SO₄, filtered and evaporated. The residue was purified by flash silica gel chromatography (ISCO®; 80 g SepaFlash® Silica Flash Column, Eluent of 0˜83% EA/PE gradient @ 80 mL/min) to give 3 (2.92 g, 55.8% yield,) as a white solid. ESI-LCMS: 500.2 [M+H]⁺; ¹H NMR (400 MHz, CDCl₃) δ=8.79 (s, 1H), 8.14 (s, 1H), 8.02 (d, J=7.6 Hz, 2H), 7.64-7.58 (m, 1H), 7.56-7.49 (m, 2H), 5.98-5.93 (m, 1H), 4.63-4.56 (m, 2H), 4.23 (s, 1H), 3.98 (dd, J=1.5, 13.1 Hz, 1H), 3.75 (dd, J=1.5, 13.1 Hz, 1H), 3.28 (s, 3H), 2.06-1.99 (m, 1H), 1.00-0.90 (m, 9H), 0.15 (d, J=7.0 Hz, 6H).

Preparation of (4): 3 (6 g, 12.01 mmol) and tert-butyl N-methylsulfonylcarbamate (3.52 g, 18.01 mmol) were co-evaporated with toluene (50 mL), dissolved in dry THF (100 mL), and cooled to 0° C. PPh₃ (9.45 g, 36.03 mmol,) was then added, followed by dropwise addition of DIAD (7.28 g, 36.03 mmol, 7.00 mL) in dry THF (30 mL). The reaction mixture was stirred at 20° C. for 18 h. Upon completion, the reaction mixture was then diluted with DCM (100 mL) and washed with water (70 mL) and brine (70 mL), dried over Na₂SO₄, filtered and evaporated to give a residue. The residue was purified by flash silica gel chromatography (ISCO®; 80 g SepaFlash® Silica Flash Column, Eluent of 0˜100% Ethyl acetate/Petroleum ether gradient @ 60 mL/min) followed by reverse-phase HPLC (0.1% NH₃.H₂O condition, eluent at 74%) to give 4 (2.88 g, 25% yield) as a white solid. ESI-LCMS: 677.1 [M+H]⁺; ¹H NMR (400 MHz, CDCl₃) δ=9.24 (s, 1H), 8.84 (s, 1H), 8.36 (s, 1H), 8.05 (br d, J=7.3 Hz, 2H), 7.66-7.42 (m, 4H), 6.16 (d, J=5.0 Hz, 1H), 4.52 (br t, J=4.5 Hz, 1H), 4.25-4.10 (m, 1H), 3.97 (br dd, J=8.0, 14.8 Hz, 1H), 3.48 (s, 3H), 3.27 (s, 3H), 1.54 (s, 9H), 0.95 (s, 9H), 0.14 (d, J=0.8 Hz, 6H).

Preparation of (5): To a solution of 4 (2.8 g, 4.14 mmol) in THF (20 mL) was added TBAF (4 M, 1.03 mL) and the mixture was stirred at 20° C. for 12 h. The reaction mixture was then evaporated. The residue was purified by flash silica gel chromatography (ISCO®; 12 g SepaFlash® Silica Flash Column, Eluent of 0˜6% MeOH/ethyl acetate gradient @ 20 mL/min) to give 5 (2.1 g, 83.92% yield) as a white solid. ESI-LCMS: 563.1[M+H]⁺; ¹H NMR (400 MHz, CDCl₃) δ=8.85-8.77 (m, 1H), 8.38 (s, 1H), 8.11-7.99 (m, 2H), 7.64-7.50 (m, 4H), 6.19 (d, J=2.8 Hz, 1H), 4.36-4.33 (m, 1H), 4.29 (br d, J=4.3 Hz, 1H), 4.22-4.02 (m, 2H), 3.65-3.59 (m, 3H), 3.28 (s, 3H), 1.54 (s, 9H).

Preparation of (6): To a solution of 5 (2.1 g, 3.73 mmol) in DCM (20 mL) was added TFA (7.70 g, 67.53 mmol, 5 mL) at 0° C. The reaction mixture was stirred at 20° C. for 24 h. Upon completion, the reaction was quenched with saturated aq. NaHCO₃ to reach pH 7. The organic layer was dried over Na₂SO₄, filtered, and evaporated at low pressure. The residue was purified by flash silica gel chromatography (ISCO®; 12 g SepaFlash® Silica Flash Column, Eluent of 0˜7% DCM/MeOH gradient @ 20 mL/min) to give 1.6 g (impure, 75% LCMS purity), followed by prep-HPLC [FA condition, column: Boston Uni C18 40*150*5 um; mobile phase: [water (0.225% FA)-ACN]; B %: 8%-38%, 7.7 min.] to give 6 (1.04 g, 63.7% yield) as a white solid. ESI-LCMS: 485.0 [M+Na]⁺; ¹H NMR (400 MHz, DMSO-d₆) δ=11.27-11.21 (m, 1H), 8.77 (s, 1H), 8.74 (s, 1H), 8.05 (d, J=7.3 Hz, 2H), 7.68-7.62 (m, 1H), 7.59-7.53 (m, 2H), 7.39 (t, J=6.3 Hz, 1H), 6.16 (d, J=6.0 Hz, 1H), 5.48 (d, J=5.5 Hz, 1H), 4.55 (t, J=5.5 Hz, 1H), 4.43-4.37 (m, 1H), 4.08-4.02 (m, 1H), 3.41-3.36 (m, 1H), 3.35 (s, 3H), 3.31-3.22 (m, 1H), 2.91 (s, 3H).

Preparation of (Example 8 monomer): To a solution of 6 (1 g, 2.16 mmol) in DCM (30 mL) was added P1 (977.58 mg, 3.24 mmol, 1.03 mL), followed by DCI (306.43 mg, 2.59 mmol) at 0° C. in one portion under Ar atmosphere. The mixture was degassed and purged with Ar for 3 times, warmed to 20° C., and stirred for 2 hr under Ar atmosphere. Upon completion as monitored by LCMS and TLC (PE:EtOAc=4:1), the reaction mixture was diluted with sat.aq. NaHCO₃ (30 mL) and extracted with DCM (50 mL*2). The combined organic layers were dried over anhydrous Na₂SO₄, filtered, and the filtrate was concentrated under reduced pressure to give a residue. The crude product was purified by reversed-phase HPLC (40 g C18 column: neutral condition, Eluent of 0˜57% of 0.3% NH₄HCO₃ in H₂O/CH₃CN ether gradient @ 35 mL/min) to give Example 8 monomer (0.49 g, 33.7% yield) as a white solid. ESI-LCMS: 663.1[M+H]⁺; ¹H NMR (400 MHz, CD₃CN) δ=1.19-1.29 (m, 12H) 2.71 (q, J=5.77 Hz, 2H) 2.94 (d, J=6.27 Hz, 3H) 3.35 (d, J=15.56 Hz, 3H) 3.40-3.52 (m, 2H) 3.61-3.97 (m, 4H) 4.23-4.45 (m, 1H) 4.55-4.74 (m, 2H) 6.02 (dd, J=10.67, 6.40 Hz, 1H) 7.25 (br s, 1H) 7.47-7.57 (m, 2H) 7.59-7.68 (m, 1H) 8.01 (d, J=7.78 Hz, 2H) 8.28 (s, 1H) 8.66 (s, 1H) 9.69 (br s, 1H); ³¹P NMR (162 MHz, CD₃CN) δ=150.92, 149.78.

### Example 9: Synthesis of 5′-stabilized End Cap Modified Oligonucleotides

This example provides an exemplary method for synthesizing the siNAs comprising a 5′-stabilized end caps disclosed herein. The 5′-stabilized end cap and/or deuterated phosphoramidites were dissolved in anhydrous acetonitrile and oligonucleotide synthesis was performed on a Expedite 8909 Synthesizer using standard phosphoramidite chemistry. An extended coupling (12 minutes) of 0.12 M solution of phosphoramidite in anhydrous CH₃CN in the presence of Benzyl-thio-tetrazole (BTT) activator to a solid bound oligonucleotide followed by standard capping, oxidation and sulfurization produced modified oligonucleotides. The 0.02 M 12, THE: Pyridine; Water 7:2:1 was used as an oxidizing agent, while DDTT (dimethylamino-methylidene) amino)-3H-1,2,4-dithiazaoline-3-thione was used as the sulfur-transfer agent for the synthesis of oligoribonucleotide with a phosphorothioate backbone. The stepwise coupling efficiency of all modified phosphoramidites was achieved around 98%. After synthesis the solid support was heated with aqueous ammonia (28%) solution at 45° C. for 16 h or 0.05 M K₂CO₃ in methanol was used to deprotect the base labile protecting groups. The crude oligonucleotides were precipitated with isopropanol and centrifuged (Eppendorf 5810R, 3000 g, 4° C., 15 min) to obtain a pellet. The crude product was then purified using ion exchange chromatography (TSK gel column, 20 mM NaH₂PO₄, 10% CH₃CN, 1 M NaBr, gradient 20-60% B over 20 column volumes) and fractions were analyzed by ion change chromatography on an HPLC. Pure fractions were pooled and desalted by Sephadex G-25 column and evaporated to dryness. The purity and molecular weight were determined by HPLC analysis and ESI-MS analysis. Single strand RNA oligonucleotides (sense and antisense strand) were annealed (1:1 by molar equivalents) at 90° C. for 3 min followed by RT 40 min) to produce the duplexes.

### Example 10: SARS-CoV-2-Nanoluc Antiviral Assay in Human ACE-2 Expressing A549 Cells

The assay has been modified from Xie X et al., 2020, Nature Communications, doi.org/10.1038/s41467-020-19055-7.

A549 cells stably expressing human ACE2 were grown in culture medium consisting of high-glucose DMEM supplemented with 10% fetal bovine serum, 1% penicillin/streptomycin, 1% HEPES and 10 μg/mL Blasticidin S. Cells were grown at 37° C. with 5% CO₂. All culture medium and antibiotics were purchased from ThermoFisher Scientific (Waltham, Mass.). SARS-CoV-2-Nluc virus was generated through inserting the nanoluciferase gene into the ORF7 gene of the infectious cDNA clone SARS-CoV-2 virus (strain 2019-nCoV/USA_WA1/2020). For SARS-CoV-2-Nluc antiviral assay, A549-hACE2 cells (12,000 cells per well in 50 ul phenol-red free medium containing 2% FBS) were plated into white opaque 96-well plate (purchased from Corning, Corning, N.Y.). On the next day, 50 ul SARS-CoV-2-Nluc virus (MOI 0.08) was added to the cells, and incubated at 37° C. with 5% CO₂ for 3 hours. Oligonucleotides were diluted in Opti-MEM medium and mixed with equal volume of diluted transfection reagent RNAiMaX (0.2 ul/well) (ThermoFisher). The transfection mixture was incubated at room temperature for 10 mins and then added to cell plate at 3 hr post infection (20 ul/well). 48 hr post infection, 60 μL Nano luciferase substrate (Promega<Madison, Wis.) were added to each well. Luciferase signals were measured using a Synergy™ Neo2 microplate reader (BioTek, Winooski, Vt.). Antiviral % inhibition was calculated as follows: [(Oligonucleotide treated cells infected sample)−(no oligonucleotide infected control)]/[(Uninfected control)−(no oligonucleotide infected control)]*100; Using GraphPad (San Diego, Calif.) prism software version 8.3.1, the antiviral dose-response plot was generated as a sigmoidal fit, log(inhibitor) vs response-variable slope (four parameters) model and the EC₅₀ was calculated which is the predicted oligonucleotide concentration corresponding to a 50% inhibition of the viral cytopathic effect.

Results for siNA assessed with this assay are shown in Table 4 at the end of the specification in column labeled “SARS-CoV-2 nanoluc hACE-2 A549 assay”.

### Example 11: Three Concentration Reporter Plasmid Luciferase and Cytotoxicity Assay in COS-7 Cells

COS-7 monkey fibroblast cells (ATCC, CRL-1651) were seeded into 96-well culture plates at 15.0×10⁴ cells/well and cultured in Dulbecco's Modified Eagle's Medium (DMEM; Hyclone, SH30022) supplemented with 10% fetal bovine serum (FBS; Sigma-Aldrich, F4135) and 1% Penicillin-Streptomycin (P/S; Corning, 30-002-CI) at 37° C. and 5% CO₂. After 6 hrs of incubation, cells were transiently transfected with psiCHECK2-SARS-CoV-2 plasmid (custom-synthesized by Genscript) at 50 ng/well using 0.3 μL of Lipofectamine 3000 transfection reagent (1:1 reagent/psi-CHECK2-SARS-CoV-2 DNA ratio; Invitrogen) in Opti-MEM (Invitrogen, 11058-021) according to the manufacturer's protocol. After overnight incubation, the medium was removed and replaced with 100 ul fresh growth media. Test siRNAs along with appropriate controls (Ambion siRNAs, ThermoFisher) were diluted to final concentration of 1, 10 or 100 nM in Opti-MEM (Invitrogen, 11058-021). Cells were then transfected with test siRNAs in duplicates using 0.3 ul/well RNAiMAX transfection reagent (1:1 ratio; Invitrogen) according to the manufacturer's protocol. After approximately 48 hrs, the culture plates were equilibrated to RT, 100 μL of Dual-Luciferase Reporter Assay reagent (Promega, E6120) were added to each well according to manufacturer's protocol. Luminescence was measured on an Envision plate reader (Perkin Elmer). The results were then quantified by calculating the ratio of renilla to firefly luciferase expression for each of the duplicates and reported as percent inhibition of luciferase activity relative to no-drug control (mock transfection with psiCHECK2-SARS-CoV-2 plasmid). The assay was repeated with a different set of plates and cytotoxicity of test siRNAs was assessed 48 hrs post treatment of COS-7 cells. The cells were lysed and assayed with Cell-Titer Glo reagent (Promega) according to the manufacturer's protocol.

Results for siNA assessed with this assay are shown in Table 4 at the end of the specification in column labeled “pSiCHECK-2 reporter assay Cos-7 at least 50% inhibition”. The data was reported as % viability relative to no-drug control (mock transfection with psiCHECK2-SARS-CoV-2 plasmid).

### Example 12: Reporter Plasmid Luciferase and Cytotoxicity Assay in Cos7 Cells

COS-7 monkey fibroblast cells (ATCC, CRL-1651) were seeded into 96-well culture plates at 15.0×10⁴ cells/well and cultured in Dulbecco's Modified Eagle's Medium (DMEM; Hyclone, SH30022) supplemented with 10% fetal bovine serum (FBS; Sigma-Aldrich, F4135) and 1% Penicillin-Streptomycin (P/S; Corning, 30-002-CI) at 37° C. and 5% CO₂. After 6 hrs of incubation, cells were transiently transfected with psiCHECK2-SARS-CoV-2 plasmid (custom-synthesized by Genscript) at 50 ng/well using 0.3 μL of Lipofectamine 3000 transfection reagent (1:1 reagent/psi-CHECK2-SARS-CoV-2 DNA ratio; Invitrogen) in Opti-MEM (Invitrogen, 11058-021) according to the manufacturer's protocol. After overnight incubation, the medium was removed and replaced with 100 ul fresh growth media. Test siRNAs along with appropriate controls (Ambion siRNAs, ThermoFisher) were serially diluted in Opti-MEM (Invitrogen, 11058-021). Cells were then transfected with test siRNAs in duplicates using 0.3 ul/well RNAiMAX transfection reagent (1:1 ratio; Invitrogen) according to the manufacturer's protocol. After approximately 48 hrs, the culture plates were equilibrated to RT, 100 μL of Dual-Luciferase Reporter Assay reagent (Promega, E6120) were added to each well according to manufacturer's protocol. Luminescence was measured on an Envision plate reader (Perkin Elmer). The results were then quantified by calculating the ratio of renilla to firefly luciferase expression for each of the duplicates and reported as percent inhibition of luciferase activity relative to no-drug control (mock transfection with psiCHECK2-SARS-CoV-2 plasmid) and dose-response curves were fitted by non-linear regression with variable slope (four parameters). Statistical analysis was performed in GraphPad Prism 8.3.1 (San Diego, Calif.) and the EC₅₀ was calculated which is the predicted oligonucleotide concentration corresponding to a 50% inhibition of the luciferase activity. The assay was repeated with a different set of plates and cytotoxicity of test siRNAs was assessed 48 hrs post treatment of COS-7 cells. The cells were lysed and assayed with Cell-Titer Glo reagent (Promega) according to the manufacturer's protocol.

Results for siNA assessed with this assay are shown in Table 4 at the end of the specification in column labeled “pSiCHECK-2 reporter assay Cos-7”. The data was reported as % viability relative to no-drug control (mock transfection with psiCHECK2-SARS-CoV-2 plasmid) and dose-response curves were fitted by non-linear regression with variable slope (four parameters) using GraphPad prism software version 8.3.1.

All patents and publications mentioned in the specification are indicative of the levels of those of ordinary skill in the art to which the disclosure pertains. All patents and publications are herein incorporated by reference to the same extent as if each individual publication was specifically and individually indicated to be incorporated by reference.

Further, one skilled in the art readily appreciates that the present disclosure is well adapted to carry out the objects and obtain the ends and advantages mentioned, as well as those inherent therein. Modifications therein and other uses will occur to those skilled in the art. These modifications are encompassed within the spirit of the disclosure and are defined by the scope of the claims, which set forth non-limiting embodiments of the disclosure.

TABLE 1
Oligonucleotide Target Sequences

| | | | | | | |
|---|---|---|---|---|---|---|
| | | | SEQ | Target | SEQ | Target reverse |
| | Start on | End on | ID | forward sequence | ID | complement sequence |
| Alias | target | target | NO | (sense strand) | NO | (antisense strand) |

19-Mer Target Sequences

| | | | | | | |
|---|---|---|---|---|---|---|
| NC_045512.2_19mer_win1_00190 | 190 | 208 | 1 | CTGCTTACGGTTTCGTCCG | 1204 | CGGACGAAACCGTAAGCAG |
| NC_045512.2_19mer_win1_00191 | 191 | 209 | 2 | TGCTTACGGTTTCGTCCGT | 1205 | ACGGACGAAACCGTAAGCA |
| NC_045512.2_19mer_win1_00192 | 192 | 210 | 3 | GCTTACGGTTTCGTCCGTG | 1206 | CACGGACGAAACCGTAAGC |
| NC_045512.2_19mer_win1_00193 | 193 | 211 | 4 | CTTACGGTTTCGTCCGTGT | 1207 | ACACGGACGAAACCGTAAG |
| NC_045512.2_19mer_win1_00194 | 194 | 212 | 5 | TTACGGTTTCGTCCGTGTT | 1208 | AACACGGACGAAACCGTAA |
| NC_045512.2_19mer_win1_00195 | 195 | 213 | 6 | TACGGTTTCGTCCGTGTTG | 1209 | CAACACGGACGAAACCGTA |
| NC_045512.2_19mer_win1_00196 | 196 | 214 | 7 | ACGGTTTCGTCCGTGTTGC | 1210 | GCAACACGGACGAAACCGT |
| NC_045512.2_19mer_win1_00197 | 197 | 215 | 8 | CGGTTTCGTCCGTGTTGCA | 1211 | TGCAACACGGACGAAACCG |
| NC_045512.2_19mer_win1_00198 | 198 | 216 | 9 | GGTTTCGTCCGTGTTGCAG | 1212 | CTGCAACACGGACGAAACC |
| NC_045512.2_19mer_win1_00233 | 233 | 251 | 10 | CTAGGTTTCGTCCGGGTGT | 1213 | ACACCCGGACGAAACCTAG |
| NC_045512.2_19mer_win1_00234 | 234 | 252 | 11 | TAGGTTTCGTCCGGGTGTG | 1214 | CACACCCGGACGAAACCTA |
| NC_045512.2_19mer_win1_00235 | 235 | 253 | 12 | AGGTTTCGTCCGGGTGTGA | 1215 | TCACACCCGGACGAAACCT |
| NC_045512.2_19mer_win1_00236 | 236 | 254 | 13 | GGTTTCGTCCGGGTGTGAC | 1216 | GTCACACCCGGACGAAACC |
| NC_045512.2_19mer_win1_00237 | 237 | 255 | 14 | GTTTCGTCCGGGTGTGACC | 1217 | GGTCACACCCGGACGAAAC |
| NC_045512.2_19mer_win1_00238 | 238 | 256 | 15 | TTTCGTCCGGGTGTGACCG | 1218 | CGGTCACACCCGGACGAAA |
| NC_045512.2_19mer_win1_00239 | 239 | 257 | 16 | TTCGTCCGGGTGTGACCGA | 1219 | TCGGTCACACCCGGACGAA |
| NC_045512.2_19mer_win1_00240 | 240 | 258 | 17 | TCGTCCGGGTGTGACCGAA | 1220 | TTCGGTCACACCCGGACGA |
| NC_045512.2_19mer_win1_00241 | 241 | 259 | 18 | CGTCCGGGTGTGACCGAAA | 1221 | TTTCGGTCACACCCGGACG |
| NC_045512.2_19mer_win1_00242 | 242 | 260 | 19 | GTCCGGGTGTGACCGAAAG | 1222 | CTTTCGGTCACACCCGGAC |
| NC_045512.2_19mer_win1_00243 | 243 | 261 | 20 | TCCGGGTGTGACCGAAAGG | 1223 | CCTTTCGGTCACACCCGGA |
| NC_045512.2_19mer_win1_00244 | 244 | 262 | 21 | CCGGGTGTGACCGAAAGGT | 1224 | ACCTTTCGGTCACACCCGG |
| NC_045512.2_19mer_win1_00245 | 245 | 263 | 22 | CGGGTGTGACCGAAAGGTA | 1225 | TACCTTTCGGTCACACCCG |
| NC_045512.2_19mer_win1_00246 | 246 | 264 | 23 | GGGTGTGACCGAAAGGTAA | 1226 | TTACCTTTCGGTCACACCC |
| NC_045512.2_19mer_win1_00247 | 247 | 265 | 24 | GGTGTGACCGAAAGGTAAG | 1227 | CTTACCTTTCGGTCACACC |
| NC_045512.2_19mer_win1_00248 | 248 | 266 | 25 | GTGTGACCGAAAGGTAAGA | 1228 | TCTTACCTTTCGGTCACAC |
| NC_045512.2_19mer_win1_00249 | 249 | 267 | 26 | TGTGACCGAAAGGTAAGAT | 1229 | ATCTTACCTTTCGGTCACA |
| NC_045512.2_19mer_win1_00250 | 250 | 268 | 27 | GTGACCGAAAGGTAAGATG | 1230 | CATCTTACCTTTCGGTCAC |
| NC_045512.2_19mer_win1_00251 | 251 | 269 | 28 | TGACCGAAAGGTAAGATGG | 1231 | CCATCTTACCTTTCGGTCA |
| NC_045512.2_19mer_win1_00252 | 252 | 270 | 29 | GACCGAAAGGTAAGATGGA | 1232 | TCCATCTTACCTTTCGGTC |
| NC_045512.2_19mer_win1_00253 | 253 | 271 | 30 | ACCGAAAGGTAAGATGGAG | 1233 | CTCCATCTTACCTTTCGGT |
| NC_045512.2_19mer_win1_00254 | 254 | 272 | 31 | CCGAAAGGTAAGATGGAGA | 1234 | TCTCCATCTTACCTTTCGG |
| NC_045512.2_19mer_win1_00255 | 255 | 273 | 32 | CGAAAGGTAAGATGGAGAG | 1235 | CTCTCCATCTTACCTTTCG |
| NC_045512.2_19mer_win1_00256 | 256 | 274 | 33 | GAAAGGTAAGATGGAGAGC | 1236 | GCTCTCCATCTTACCTTTC |
| NC_045512.2_19mer_win1_00257 | 257 | 275 | 34 | AAAGGTAAGATGGAGAGCC | 1237 | GGCTCTCCATCTTACCTTT |
| NC_045512.2_19mer_win1_00258 | 258 | 276 | 35 | AAGGTAAGATGGAGAGCCT | 1238 | AGGCTCTCCATCTTACCTT |
| NC_045512.2_19mer_win1_00259 | 259 | 277 | 36 | AGGTAAGATGGAGAGCCTT | 1239 | AAGGCTCTCCATCTTACCT |
| NC_045512.2_19mer_win1_00260 | 260 | 278 | 37 | GGTAAGATGGAGAGCCTTG | 1240 | CAAGGCTCTCCATCTTACC |
| NC_045512.2_19mer_win1_00261 | 261 | 279 | 38 | GTAAGATGGAGAGCCTTGT | 1241 | ACAAGGCTCTCCATCTTAC |
| NC_045512.2_19mer_win1_00288 | 288 | 306 | 39 | TCAACGAGAAAACACACGT | 1242 | ACGTGTGTTTTCTCGTTGA |
| NC_045512.2_19mer_win1_00289 | 289 | 307 | 40 | CAACGAGAAAACACACGTC | 1243 | GACGTGTGTTTTCTCGTTG |
| NC_045512.2_19mer_win1_00290 | 290 | 308 | 41 | AACGAGAAAACACACGTCC | 1244 | GGACGTGTGTTTTCTCGTT |
| NC_045512.2_19mer_win1_00291 | 291 | 309 | 42 | ACGAGAAAACACACGTCCA | 1245 | TGGACGTGTGTTTTCTCGT |
| NC_045512.2_19mer_win1_00292 | 292 | 310 | 43 | CGAGAAAACACACGTCCAA | 1246 | TTGGACGTGTGTTTTCTCG |
| NC_045512.2_19mer_win1_00293 | 293 | 311 | 44 | GAGAAAACACACGTCCAAC | 1247 | GTTGGACGTGTGTTTTCTC |
| NC_045512.2_19mer_win1_00294 | 294 | 312 | 45 | AGAAAACACACGTCCAACT | 1248 | AGTTGGACGTGTGTTTTCT |
| NC_045512.2_19mer_win1_00295 | 295 | 313 | 46 | GAAAACACACGTCCAACTC | 1249 | GAGTTGGACGTGTGTTTTC |
| NC_045512.2_19mer_win1_00296 | 296 | 314 | 47 | AAAACACACGTCCAACTCA | 1250 | TGAGTTGGACGTGTGTTTT |
| NC_045512.2_19mer_win1_00297 | 297 | 315 | 48 | AAACACACGTCCAACTCAG | 1251 | CTGAGTTGGACGTGTGTTT |
| NC_045512.2_19mer_win1_00298 | 298 | 316 | 49 | AACACACGTCCAACTCAGT | 1252 | ACTGAGTTGGACGTGTGTT |
| NC_045512.2_19mer_win1_00299 | 299 | 317 | 50 | ACACACGTCCAACTCAGTT | 1253 | AACTGAGTTGGACGTGTGT |
| NC_045512.2_19mer_win1_00300 | 300 | 318 | 51 | CACACGTCCAACTCAGTTT | 1254 | AAACTGAGTTGGACGTGTG |
| NC_045512.2_19mer_win1_00301 | 301 | 319 | 52 | ACACGTCCAACTCAGTTTG | 1255 | CAAACTGAGTTGGACGTGT |
| NC_045512.2_19mer_win1_00302 | 302 | 320 | 53 | CACGTCCAACTCAGTTTGC | 1256 | GCAAACTGAGTTGGACGTG |
| NC_045512.2_19mer_win1_00303 | 303 | 321 | 54 | ACGTCCAACTCAGTTTGCC | 1257 | GGCAAACTGAGTTGGACGT |
| NC_045512.2_19mer_win1_00304 | 304 | 322 | 55 | CGTCCAACTCAGTTTGCCT | 1258 | AGGCAAACTGAGTTGGACG |
| NC_045512.2_19mer_win1_00305 | 305 | 323 | 56 | GTCCAACTCAGTTTGCCTG | 1259 | CAGGCAAACTGAGTTGGAC |
| NC_045512.2_19mer_win1_00306 | 306 | 324 | 57 | TCCAACTCAGTTTGCCTGT | 1260 | ACAGGCAAACTGAGTTGGA |
| NC_045512.2_19mer_win1_00455 | 455 | 473 | 58 | CTTGAACAGCCCTATGTGT | 1261 | ACACATAGGGCTGTTCAAG |
| NC_045512.2_19mer_win1_00456 | 456 | 474 | 59 | TTGAACAGCCCTATGTGTT | 1262 | AACACATAGGGCTGTTCAA |
| NC_045512.2_19mer_win1_00457 | 457 | 475 | 60 | TGAACAGCCCTATGTGTTC | 1263 | GAACACATAGGGCTGTTCA |
| NC_045512.2_19mer_win1_00458 | 458 | 476 | 61 | GAACAGCCCTATGTGTTCA | 1264 | TGAACACATAGGGCTGTTC |
| NC_045512.2_19mer_win1_00459 | 459 | 477 | 62 | AACAGCCCTATGTGTTCAT | 1265 | ATGAACACATAGGGCTGTT |
| NC_045512.2_19mer_win1_00626 | 626 | 644 | 63 | GTTCTTCTTCGTAAGAACG | 1266 | CGTTCTTACGAAGAAGAAC |
| NC_045512.2_19mer_win1_00627 | 627 | 645 | 64 | TTCTTCTTCGTAAGAACGG | 1267 | CCGTTCTTACGAAGAAGAA |
| NC_045512.2_19mer_win1_00628 | 628 | 646 | 65 | TCTTCTTCGTAAGAACGGT | 1268 | ACCGTTCTTACGAAGAAGA |
| NC_045512.2_19mer_win1_00629 | 629 | 647 | 66 | CTTCTTCGTAAGAACGGTA | 1269 | TACCGTTCTTACGAAGAAG |
| NC_045512.2_19mer_win1_00630 | 630 | 648 | 67 | TTCTTCGTAAGAACGGTAA | 1270 | TTACCGTTCTTACGAAGAA |
| NC_045512.2_19mer_win1_00631 | 631 | 649 | 68 | TCTTCGTAAGAACGGTAAT | 1271 | ATTACCGTTCTTACGAAGA |
| NC_045512.2_19mer_win1_00632 | 632 | 650 | 69 | CTTCGTAAGAACGGTAATA | 1272 | TATTACCGTTCTTACGAAG |
| NC_045512.2_19mer_win1_00633 | 633 | 651 | 70 | TTCGTAAGAACGGTAATAA | 1273 | TTATTACCGTTCTTACGAA |
| NC_045512.2_19mer_win1_00704 | 704 | 722 | 71 | GACGAGCTTGGCACTGATC | 1274 | GATCAGTGCCAAGCTCGTC |
| NC_045512.2_19mer_win1_00705 | 705 | 723 | 72 | ACGAGCTTGGCACTGATCC | 1275 | GGATCAGTGCCAAGCTCGT |
| NC_045512.2_19mer_win1_03352 | 3352 | 3370 | 73 | TGGTTATTTAAAACTTACT | 1276 | AGTAAGTTTTAAATAACCA |
| NC_045512.2_19mer_win1_03353 | 3353 | 3371 | 74 | GGTTATTTAAAACTTACTG | 1277 | CAGTAAGTTTTAAATAACC |
| NC_045512.2_19mer_win1_03354 | 3354 | 3372 | 75 | GTTATTTAAAACTTACTGA | 1278 | TCAGTAAGTTTTAAATAAC |
| NC_045512.2_19mer_win1_03355 | 3355 | 3373 | 76 | TTATTTAAAACTTACTGAC | 1279 | GTCAGTAAGTTTTAAATAA |
| NC_045512.2_19mer_win1_03356 | 3356 | 3374 | 77 | TATTTAAAACTTACTGACA | 1280 | TGTCAGTAAGTTTTAAATA |
| NC_045512.2_19mer_win1_03357 | 3357 | 3375 | 78 | ATTTAAAACTTACTGACAA | 1281 | TTGTCAGTAAGTTTTAAAT |
| NC_045512.2_19mer_win1_03358 | 3358 | 3376 | 79 | TTTAAAACTTACTGACAAT | 1282 | ATTGTCAGTAAGTTTTAAA |
| NC_045512.2_19mer_win1_03359 | 3359 | 3377 | 80 | TTAAAACTTACTGACAATG | 1283 | CATTGTCAGTAAGTTTTAA |
| NC_045512.2_19mer_win1_03360 | 3360 | 3378 | 81 | TAAAACTTACTGACAATGT | 1284 | ACATTGTCAGTAAGTTTTA |
| NC_045512.2_19mer_win1_05384 | 5384 | 5402 | 82 | GCTGCTAACTTTTGTGCAC | 1285 | GTGCACAAAAGTTAGCAGC |
| NC_045512.2_19mer_win1_05385 | 5385 | 5403 | 83 | CTGCTAACTTTTGTGCACT | 1286 | AGTGCACAAAAGTTAGCAG |
| NC_045512.2_19mer_win1_06406 | 6406 | 6424 | 84 | CTCTGAAGAAGTAGTGGAA | 1287 | TTCCACTACTTCTTCAGAG |
| NC_045512.2_19mer_win1_06407 | 6407 | 6425 | 85 | TCTGAAGAAGTAGTGGAAA | 1288 | TTTCCACTACTTCTTCAGA |
| NC_045512.2_19mer_win1_06408 | 6408 | 6426 | 86 | CTGAAGAAGTAGTGGAAAA | 1289 | TTTTCCACTACTTCTTCAG |
| NC_045512.2_19mer_win1_06409 | 6409 | 6427 | 87 | TGAAGAAGTAGTGGAAAAT | 1290 | ATTTTCCACTACTTCTTCA |
| NC_045512.2_19mer_win1_06410 | 6410 | 6428 | 88 | GAAGAAGTAGTGGAAAATC | 1291 | GATTTTCCACTACTTCTTC |
| NC_045512.2_19mer_win1_06411 | 6411 | 6429 | 89 | AAGAAGTAGTGGAAAATCC | 1292 | GGATTTTCCACTACTTCTT |
| NC_045512.2_19mer_win1_06412 | 6412 | 6430 | 90 | AGAAGTAGTGGAAAATCCT | 1293 | AGGATTTTCCACTACTTCT |
| NC_045512.2_19mer_win1_06413 | 6413 | 6431 | 91 | GAAGTAGTGGAAAATCCTA | 1294 | TAGGATTTTCCACTACTTC |
| NC_045512.2_19mer_win1_06414 | 6414 | 6432 | 92 | AAGTAGTGGAAAATCCTAC | 1295 | GTAGGATTTTCCACTACTT |
| NC_045512.2_19mer_win1_06415 | 6415 | 6433 | 93 | AGTAGTGGAAAATCCTACC | 1296 | GGTAGGATTTTCCACTACT |
| NC_045512.2_19mer_win1_06461 | 6461 | 6479 | 94 | GTGAAAACTACCGAAGTTG | 1297 | CAACTTCGGTAGTTTTCAC |
| NC_045512.2_19mer_win1_06462 | 6462 | 6480 | 95 | TGAAAACTACCGAAGTTGT | 1298 | ACAACTTCGGTAGTTTTCA |
| NC_045512.2_19mer_win1_06463 | 6463 | 6481 | 96 | GAAAACTACCGAAGTTGTA | 1299 | TACAACTTCGGTAGTTTTC |
| NC_045512.2_19mer_win1_06464 | 6464 | 6482 | 97 | AAAACTACCGAAGTTGTAG | 1300 | CTACAACTTCGGTAGTTTT |
| NC_045512.2_19mer_win1_06465 | 6465 | 6483 | 98 | AAACTACCGAAGTTGTAGG | 1301 | CCTACAACTTCGGTAGTTT |
| NC_045512.2_19mer_win1_07532 | 7532 | 7550 | 99 | TGTACAACTATTGTTAATG | 1302 | CATTAACAATAGTTGTACA |
| NC_045512.2_19mer_win1_07533 | 7533 | 7551 | 100 | GTACAACTATTGTTAATGG | 1303 | CCATTAACAATAGTTGTAC |
| NC_045512.2_19mer_win1_09588 | 9588 | 9606 | 101 | TTTACTTGTACTTGACATT | 1304 | AATGTCAAGTACAAGTAAA |
| NC_045512.2_19mer_win1_10484 | 10484 | 10502 | 102 | TCATGTGGTAGTGTTGGTT | 1305 | AACCAACACTACCACATGA |
| NC_045512.2_19mer_win1_10485 | 10485 | 10503 | 103 | CATGTGGTAGTGTTGGTTT | 1306 | AAACCAACACTACCACATG |
| NC_045512.2_19mer_win1_10486 | 10486 | 10504 | 104 | ATGTGGTAGTGTTGGTTTT | 1307 | AAAACCAACACTACCACAT |
| NC_045512.2_19mer_win1_10487 | 10487 | 10505 | 105 | TGTGGTAGTGTTGGTTTTA | 1308 | TAAAACCAACACTACCACA |
| NC_045512.2_19mer_win1_10488 | 10488 | 10506 | 106 | GTGGTAGTGTTGGTTTTAA | 1309 | TTAAAACCAACACTACCAC |
| NC_045512.2_19mer_win1_10489 | 10489 | 10507 | 107 | TGGTAGTGTTGGTTTTAAC | 1310 | GTTAAAACCAACACTACCA |
| NC_045512.2_19mer_win1_10490 | 10490 | 10508 | 108 | GGTAGTGTTGGTTTTAACA | 1311 | TGTTAAAACCAACACTACC |
| NC_045512.2_19mer_win1_10491 | 10491 | 10509 | 109 | GTAGTGTTGGTTTTAACAT | 1312 | ATGTTAAAACCAACACTAC |
| NC_045512.2_19mer_win1_11609 | 11609 | 11627 | 110 | GTTTATTGTTTCTTAGGCT | 1313 | AGCCTAAGAAACAATAAAC |
| NC_045512.2_19mer_win1_11610 | 11610 | 11628 | 111 | TTTATTGTTTCTTAGGCTA | 1314 | TAGCCTAAGAAACAATAAA |
| NC_045512.2_19mer_win1_11611 | 11611 | 11629 | 112 | TTATTGTTTCTTAGGCTAT | 1315 | ATAGCCTAAGAAACAATAA |
| NC_045512.2_19mer_win1_11612 | 11612 | 11630 | 113 | TATTGTTTCTTAGGCTATT | 1316 | AATAGCCTAAGAAACAATA |
| NC_045512.2_19mer_win1_11834 | 11834 | 11852 | 114 | ACTGTACAGTCTAAAATGT | 1317 | ACATTTTAGACTGTACAGT |
| NC_045512.2_19mer_win1_11835 | 11835 | 11853 | 115 | CTGTACAGTCTAAAATGTC | 1318 | GACATTTTAGACTGTACAG |
| NC_045512.2_19mer_win1_12023 | 12023 | 12041 | 116 | TCCATGCAGGGTGCTGTAG | 1319 | CTACAGCACCCTGCATGGA |
| NC_045512.2_19mer_win1_12024 | 12024 | 12042 | 117 | CCATGCAGGGTGCTGTAGA | 1320 | TCTACAGCACCCTGCATGG |
| NC_045512.2_19mer_win1_12025 | 12025 | 12043 | 118 | CATGCAGGGTGCTGTAGAC | 1321 | GTCTACAGCACCCTGCATG |
| NC_045512.2_19mer_win1_12026 | 12026 | 12044 | 119 | ATGCAGGGTGCTGTAGACA | 1322 | TGTCTACAGCACCCTGCAT |
| NC_045512.2_19mer_win1_12027 | 12027 | 12045 | 120 | TGCAGGGTGCTGTAGACAT | 1323 | ATGTCTACAGCACCCTGCA |
| NC_045512.2_19mer_win1_12212 | 12212 | 12230 | 121 | TCTTTGAATGTGGCTAAAT | 1324 | ATTTAGCCACATTCAAAGA |
| NC_045512.2_19mer_win1_12213 | 12213 | 12231 | 122 | CTTTGAATGTGGCTAAATC | 1325 | GATTTAGCCACATTCAAAG |
| NC_045512.2_19mer_win1_12214 | 12214 | 12232 | 123 | TTTGAATGTGGCTAAATCT | 1326 | AGATTTAGCCACATTCAAA |
| NC_045512.2_19mer_win1_12215 | 12215 | 12233 | 124 | TTGAATGTGGCTAAATCTG | 1327 | CAGATTTAGCCACATTCAA |
| NC_045512.2_19mer_win1_12216 | 12216 | 12234 | 125 | TGAATGTGGCTAAATCTGA | 1328 | TCAGATTTAGCCACATTCA |
| NC_045512.2_19mer_win1_12401 | 12401 | 12419 | 126 | AACAACATTATCAACAATG | 1329 | CATTGTTGATAATGTTGTT |
| NC_045512.2_19mer_win1_12402 | 12402 | 12420 | 127 | ACAACATTATCAACAATGC | 1330 | GCATTGTTGATAATGTTGT |
| NC_045512.2_19mer_win1_12839 | 12839 | 12857 | 128 | AAATGGGCTAGATTCCCTA | 1331 | TAGGGAATCTAGCCCATTT |
| NC_045512.2_19mer_win1_12840 | 12840 | 12858 | 129 | AATGGGCTAGATTCCCTAA | 1332 | TTAGGGAATCTAGCCCATT |
| NC_045512.2_19mer_win1_12841 | 12841 | 12859 | 130 | ATGGGCTAGATTCCCTAAG | 1333 | CTTAGGGAATCTAGCCCAT |
| NC_045512.2_19mer_win1_12842 | 12842 | 12860 | 131 | TGGGCTAGATTCCCTAAGA | 1334 | TCTTAGGGAATCTAGCCCA |
| NC_045512.2_19mer_win1_12843 | 12843 | 12861 | 132 | GGGCTAGATTCCCTAAGAG | 1335 | CTCTTAGGGAATCTAGCCC |
| NC_045512.2_19mer_win1_12844 | 12844 | 12862 | 133 | GGCTAGATTCCCTAAGAGT | 1336 | ACTCTTAGGGAATCTAGCC |
| NC_045512.2_19mer_win1_12845 | 12845 | 12863 | 134 | GCTAGATTCCCTAAGAGTG | 1337 | CACTCTTAGGGAATCTAGC |
| NC_045512.2_19mer_win1_12846 | 12846 | 12864 | 135 | CTAGATTCCCTAAGAGTGA | 1338 | TCACTCTTAGGGAATCTAG |
| NC_045512.2_19mer_win1_12847 | 12847 | 12865 | 136 | TAGATTCCCTAAGAGTGAT | 1339 | ATCACTCTTAGGGAATCTA |
| NC_045512.2_19mer_win1_12848 | 12848 | 12866 | 137 | AGATTCCCTAAGAGTGATG | 1340 | CATCACTCTTAGGGAATCT |
| NC_045512.2_19mer_win1_12849 | 12849 | 12867 | 138 | GATTCCCTAAGAGTGATGG | 1341 | CCATCACTCTTAGGGAATC |
| NC_045512.2_19mer_win1_12885 | 12885 | 12903 | 139 | CAGAACTGGAACCACCTTG | 1342 | CAAGGTGGTTCCAGTTCTG |
| NC_045512.2_19mer_win1_12886 | 12886 | 12904 | 140 | AGAACTGGAACCACCTTGT | 1343 | ACAAGGTGGTTCCAGTTCT |
| NC_045512.2_19mer_win1_12887 | 12887 | 12905 | 141 | GAACTGGAACCACCTTGTA | 1344 | TACAAGGTGGTTCCAGTTC |
| NC_045512.2_19mer_win1_12888 | 12888 | 12906 | 142 | AACTGGAACCACCTTGTAG | 1345 | CTACAAGGTGGTTCCAGTT |
| NC_045512.2_19mer_win1_12889 | 12889 | 12907 | 143 | ACTGGAACCACCTTGTAGG | 1346 | CCTACAAGGTGGTTCCAGT |
| NC_045512.2_19mer_win1_12890 | 12890 | 12908 | 144 | CTGGAACCACCTTGTAGGT | 1347 | ACCTACAAGGTGGTTCCAG |
| NC_045512.2_19mer_win1_12891 | 12891 | 12909 | 145 | TGGAACCACCTTGTAGGTT | 1348 | AACCTACAAGGTGGTTCCA |
| NC_045512.2_19mer_win1_12892 | 12892 | 12910 | 146 | GGAACCACCTTGTAGGTTT | 1349 | AAACCTACAAGGTGGTTCC |
| NC_045512.2_19mer_win1_12893 | 12893 | 12911 | 147 | GAACCACCTTGTAGGTTTG | 1350 | CAAACCTACAAGGTGGTTC |
| NC_045512.2_19mer_win1_12894 | 12894 | 12912 | 148 | AACCACCTTGTAGGTTTGT | 1351 | ACAAACCTACAAGGTGGTT |
| NC_045512.2_19mer_win1_12895 | 12895 | 12913 | 149 | ACCACCTTGTAGGTTTGTT | 1352 | AACAAACCTACAAGGTGGT |
| NC_045512.2_19mer_win1_12896 | 12896 | 12914 | 150 | CCACCTTGTAGGTTTGTTA | 1353 | TAACAAACCTACAAGGTGG |
| NC_045512.2_19mer_win1_12897 | 12897 | 12915 | 151 | CACCTTGTAGGTTTGTTAC | 1354 | GTAACAAACCTACAAGGTG |
| NC_045512.2_19mer_win1_12898 | 12898 | 12916 | 152 | ACCTTGTAGGTTTGTTACA | 1355 | TGTAACAAACCTACAAGGT |
| NC_045512.2_19mer_win1_12899 | 12899 | 12917 | 153 | CCTTGTAGGTTTGTTACAG | 1356 | CTGTAACAAACCTACAAGG |
| NC_045512.2_19mer_win1_12900 | 12900 | 12918 | 154 | CTTGTAGGTTTGTTACAGA | 1357 | TCTGTAACAAACCTACAAG |
| NC_045512.2_19mer_win1_12901 | 12901 | 12919 | 155 | TTGTAGGTTTGTTACAGAC | 1358 | GTCTGTAACAAACCTACAA |
| NC_045512.2_19mer_win1_12902 | 12902 | 12920 | 156 | TGTAGGTTTGTTACAGACA | 1359 | TGTCTGTAACAAACCTACA |
| NC_045512.2_19mer_win1_12903 | 12903 | 12921 | 157 | GTAGGTTTGTTACAGACAC | 1360 | GTGTCTGTAACAAACCTAC |
| NC_045512.2_19mer_win1_12904 | 12904 | 12922 | 158 | TAGGTTTGTTACAGACACA | 1361 | TGTGTCTGTAACAAACCTA |
| NC_045512.2_19mer_win1_12905 | 12905 | 12923 | 159 | AGGTTTGTTACAGACACAC | 1362 | GTGTGTCTGTAACAAACCT |
| NC_045512.2_19mer_win1_12906 | 12906 | 12924 | 160 | GGTTTGTTACAGACACACC | 1363 | GGTGTGTCTGTAACAAACC |
| NC_045512.2_19mer_win1_12966 | 12966 | 12984 | 161 | TAAACAACCTAAATAGAGG | 1364 | CCTCTATTTAGGTTGTTTA |
| NC_045512.2_19mer_win1_12967 | 12967 | 12985 | 162 | AAACAACCTAAATAGAGGT | 1365 | ACCTCTATTTAGGTTGTTT |
| NC_045512.2_19mer_win1_12968 | 12968 | 12986 | 163 | AACAACCTAAATAGAGGTA | 1366 | TACCTCTATTTAGGTTGTT |
| NC_045512.2_19mer_win1_12969 | 12969 | 12987 | 164 | ACAACCTAAATAGAGGTAT | 1367 | ATACCTCTATTTAGGTTGT |
| NC_045512.2_19mer_win1_12970 | 12970 | 12988 | 165 | CAACCTAAATAGAGGTATG | 1368 | CATACCTCTATTTAGGTTG |
| NC_045512.2_19mer_win1_12971 | 12971 | 12989 | 166 | AACCTAAATAGAGGTATGG | 1369 | CCATACCTCTATTTAGGTT |
| NC_045512.2_19mer_win1_12972 | 12972 | 12990 | 167 | ACCTAAATAGAGGTATGGT | 1370 | ACCATACCTCTATTTAGGT |
| NC_045512.2_19mer_win1_13151 | 13151 | 13169 | 168 | AAGATGTTGTGTACACACA | 1371 | TGTGTGTACACAACATCTT |
| NC_045512.2_19mer_win1_13152 | 13152 | 13170 | 169 | AGATGTTGTGTACACACAC | 1372 | GTGTGTGTACACAACATCT |
| NC_045512.2_19mer_win1_13153 | 13153 | 13171 | 170 | GATGTTGTGTACACACACT | 1373 | AGTGTGTGTACACAACATC |
| NC_045512.2_19mer_win1_13154 | 13154 | 13172 | 171 | ATGTTGTGTACACACACTG | 1374 | CAGTGTGTGTACACAACAT |
| NC_045512.2_19mer_win1_13155 | 13155 | 13173 | 172 | TGTTGTGTACACACACTGG | 1375 | CCAGTGTGTGTACACAACA |
| NC_045512.2_19mer_win1_13156 | 13156 | 13174 | 173 | GTTGTGTACACACACTGGT | 1376 | ACCAGTGTGTGTACACAAC |
| NC_045512.2_19mer_win1_13157 | 13157 | 13175 | 174 | TTGTGTACACACACTGGTA | 1377 | TACCAGTGTGTGTACACAA |
| NC_045512.2_19mer_win1_13158 | 13158 | 13176 | 175 | TGTGTACACACACTGGTAC | 1378 | GTACCAGTGTGTGTACACA |
| NC_045512.2_19mer_win1_13363 | 13363 | 13381 | 176 | AAACACAGTCTGTACCGTC | 1379 | GACGGTACAGACTGTGTTT |
| NC_045512.2_19mer_win1_13364 | 13364 | 13382 | 177 | AACACAGTCTGTACCGTCT | 1380 | AGACGGTACAGACTGTGTT |
| NC_045512.2_19mer_win1_13365 | 13365 | 13383 | 178 | ACACAGTCTGTACCGTCTG | 1381 | CAGACGGTACAGACTGTGT |
| NC_045512.2_19mer_win1_13366 | 13366 | 13384 | 179 | CACAGTCTGTACCGTCTGC | 1382 | GCAGACGGTACAGACTGTG |
| NC_045512.2_19mer_win1_13367 | 13367 | 13385 | 180 | ACAGTCTGTACCGTCTGCG | 1383 | CGCAGACGGTACAGACTGT |
| NC_045512.2_19mer_win1_13368 | 13368 | 13386 | 181 | CAGTCTGTACCGTCTGCGG | 1384 | CCGCAGACGGTACAGACTG |
| NC_045512.2_19mer_win1_13388 | 13388 | 13406 | 182 | ATGTGGAAAGGTTATGGCT | 1385 | AGCCATAACCTTTCCACAT |
| NC_045512.2_19mer_win1_13389 | 13389 | 13407 | 183 | TGTGGAAAGGTTATGGCTG | 1386 | CAGCCATAACCTTTCCACA |
| NC_045512.2_19mer_win1_13390 | 13390 | 13408 | 184 | GTGGAAAGGTTATGGCTGT | 1387 | ACAGCCATAACCTTTCCAC |
| NC_045512.2_19mer_win1_13391 | 13391 | 13409 | 185 | TGGAAAGGTTATGGCTGTA | 1388 | TACAGCCATAACCTTTCCA |
| NC_045512.2_19mer_win1_13392 | 13392 | 13410 | 186 | GGAAAGGTTATGGCTGTAG | 1389 | CTACAGCCATAACCTTTCC |
| NC_045512.2_19mer_win1_13393 | 13393 | 13411 | 187 | GAAAGGTTATGGCTGTAGT | 1390 | ACTACAGCCATAACCTTTC |
| NC_045512.2_19mer_win1_13394 | 13394 | 13412 | 188 | AAAGGTTATGGCTGTAGTT | 1391 | AACTACAGCCATAACCTTT |
| NC_045512.2_19mer_win1_13395 | 13395 | 13413 | 189 | AAGGTTATGGCTGTAGTTG | 1392 | CAACTACAGCCATAACCTT |
| NC_045512.2_19mer_win1_13396 | 13396 | 13414 | 190 | AGGTTATGGCTGTAGTTGT | 1393 | ACAACTACAGCCATAACCT |
| NC_045512.2_19mer_win1_13397 | 13397 | 13415 | 191 | GGTTATGGCTGTAGTTGTG | 1394 | CACAACTACAGCCATAACC |
| NC_045512.2_19mer_win1_13398 | 13398 | 13416 | 192 | GTTATGGCTGTAGTTGTGA | 1395 | TCACAACTACAGCCATAAC |
| NC_045512.2_19mer_win1_13458 | 13458 | 13476 | 193 | CGTTTTTAAACGGGTTTGC | 1396 | GCAAACCCGTTTAAAAACG |
| NC_045512.2_19mer_win1_13459 | 13459 | 13477 | 194 | GTTTTTAAACGGGTTTGCG | 1397 | CGCAAACCCGTTTAAAAAC |
| NC_045512.2_19mer_win1_13460 | 13460 | 13478 | 195 | TTTTTAAACGGGTTTGCGG | 1398 | CCGCAAACCCGTTTAAAAA |
| NC_045512.2_19mer_win1_13461 | 13461 | 13479 | 196 | TTTTAAACGGGTTTGCGGT | 1399 | ACCGCAAACCCGTTTAAAA |
| NC_045512.2_19mer_win1_13462 | 13462 | 13480 | 197 | TTTAAACGGGTTTGCGGTG | 1400 | CACCGCAAACCCGTTTAAA |
| NC_045512.2_19mer_win1_13463 | 13463 | 13481 | 198 | TTAAACGGGTTTGCGGTGT | 1401 | ACACCGCAAACCCGTTTAA |
| NC_045512.2_19mer_win1_13464 | 13464 | 13482 | 199 | TAAACGGGTTTGCGGTGTA | 1402 | TACACCGCAAACCCGTTTA |
| NC_045512.2_19mer_win1_13465 | 13465 | 13483 | 200 | AAACGGGTTTGCGGTGTAA | 1403 | TTACACCGCAAACCCGTTT |
| NC_045512.2_19mer_win1_13466 | 13466 | 13484 | 201 | AACGGGTTTGCGGTGTAAG | 1404 | CTTACACCGCAAACCCGTT |
| NC_045512.2_19mer_win1_13467 | 13467 | 13485 | 202 | ACGGGTTTGCGGTGTAAGT | 1405 | ACTTACACCGCAAACCCGT |
| NC_045512.2_19mer_win1_13468 | 13468 | 13486 | 203 | CGGGTTTGCGGTGTAAGTG | 1406 | CACTTACACCGCAAACCCG |
| NC_045512.2_19mer_win1_13469 | 13469 | 13487 | 204 | GGGTTTGCGGTGTAAGTGC | 1407 | GCACTTACACCGCAAACCC |
| NC_045512.2_19mer_win1_13470 | 13470 | 13488 | 205 | GGTTTGCGGTGTAAGTGCA | 1408 | TGCACTTACACCGCAAACC |
| NC_045512.2_19mer_win1_13471 | 13471 | 13489 | 206 | GTTTGCGGTGTAAGTGCAG | 1409 | CTGCACTTACACCGCAAAC |
| NC_045512.2_19mer_win1_13472 | 13472 | 13490 | 207 | TTTGCGGTGTAAGTGCAGC | 1410 | GCTGCACTTACACCGCAAA |
| NC_045512.2_19mer_win1_13473 | 13473 | 13491 | 208 | TTGCGGTGTAAGTGCAGCC | 1411 | GGCTGCACTTACACCGCAA |
| NC_045512.2_19mer_win1_13474 | 13474 | 13492 | 209 | TGCGGTGTAAGTGCAGCCC | 1412 | GGGCTGCACTTACACCGCA |
| NC_045512.2_19mer_win1_13475 | 13475 | 13493 | 210 | GCGGTGTAAGTGCAGCCCG | 1413 | CGGGCTGCACTTACACCGC |
| NC_045512.2_19mer_win1_13476 | 13476 | 13494 | 211 | CGGTGTAAGTGCAGCCCGT | 1414 | ACGGGCTGCACTTACACCG |
| NC_045512.2_19mer_win1_13477 | 13477 | 13495 | 212 | GGTGTAAGTGCAGCCCGTC | 1415 | GACGGGCTGCACTTACACC |
| NC_045512.2_19mer_win1_13478 | 13478 | 13496 | 213 | GTGTAAGTGCAGCCCGTCT | 1416 | AGACGGGCTGCACTTACAC |
| NC_045512.2_19mer_win1_13479 | 13479 | 13497 | 214 | TGTAAGTGCAGCCCGTCTT | 1417 | AAGACGGGCTGCACTTACA |
| NC_045512.2_19mer_win1_13480 | 13480 | 13498 | 215 | GTAAGTGCAGCCCGTCTTA | 1418 | TAAGACGGGCTGCACTTAC |
| NC_045512.2_19mer_win1_13481 | 13481 | 13499 | 216 | TAAGTGCAGCCCGTCTTAC | 1419 | GTAAGACGGGCTGCACTTA |
| NC_045512.2_19mer_win1_13482 | 13482 | 13500 | 217 | AAGTGCAGCCCGTCTTACA | 1420 | TGTAAGACGGGCTGCACTT |
| NC_045512.2_19mer_win1_13483 | 13483 | 13501 | 218 | AGTGCAGCCCGTCTTACAC | 1421 | GTGTAAGACGGGCTGCACT |
| NC_045512.2_19mer_win1_13484 | 13484 | 13502 | 219 | GTGCAGCCCGTCTTACACC | 1422 | GGTGTAAGACGGGCTGCAC |
| NC_045512.2_19mer_win1_13485 | 13485 | 13503 | 220 | TGCAGCCCGTCTTACACCG | 1423 | CGGTGTAAGACGGGCTGCA |
| NC_045512.2_19mer_win1_13486 | 13486 | 13504 | 221 | GCAGCCCGTCTTACACCGT | 1424 | ACGGTGTAAGACGGGCTGC |
| NC_045512.2_19mer_win1_13487 | 13487 | 13505 | 222 | CAGCCCGTCTTACACCGTG | 1425 | CACGGTGTAAGACGGGCTG |
| NC_045512.2_19mer_win1_13488 | 13488 | 13506 | 223 | AGCCCGTCTTACACCGTGC | 1426 | GCACGGTGTAAGACGGGCT |
| NC_045512.2_19mer_win1_13489 | 13489 | 13507 | 224 | GCCCGTCTTACACCGTGCG | 1427 | CGCACGGTGTAAGACGGGC |
| NC_045512.2_19mer_win1_13490 | 13490 | 13508 | 225 | CCCGTCTTACACCGTGCGG | 1428 | CCGCACGGTGTAAGACGGG |
| NC_045512.2_19mer_win1_13491 | 13491 | 13509 | 226 | CCGTCTTACACCGTGCGGC | 1429 | GCCGCACGGTGTAAGACGG |
| NC_045512.2_19mer_win1_13492 | 13492 | 13510 | 227 | CGTCTTACACCGTGCGGCA | 1430 | TGCCGCACGGTGTAAGACG |
| NC_045512.2_19mer_win1_13493 | 13493 | 13511 | 228 | GTCTTACACCGTGCGGCAC | 1431 | GTGCCGCACGGTGTAAGAC |
| NC_045512.2_19mer_win1_13494 | 13494 | 13512 | 229 | TCTTACACCGTGCGGCACA | 1432 | TGTGCCGCACGGTGTAAGA |
| NC_045512.2_19mer_win1_13495 | 13495 | 13513 | 230 | CTTACACCGTGCGGCACAG | 1433 | CTGTGCCGCACGGTGTAAG |
| NC_045512.2_19mer_win1_13496 | 13496 | 13514 | 231 | TTACACCGTGCGGCACAGG | 1434 | CCTGTGCCGCACGGTGTAA |
| NC_045512.2_19mer_win1_13497 | 13497 | 13515 | 232 | TACACCGTGCGGCACAGGC | 1435 | GCCTGTGCCGCACGGTGTA |
| NC_045512.2_19mer_win1_13498 | 13498 | 13516 | 233 | ACACCGTGCGGCACAGGCA | 1436 | TGCCTGTGCCGCACGGTGT |
| NC_045512.2_19mer_win1_13499 | 13499 | 13517 | 234 | CACCGTGCGGCACAGGCAC | 1437 | GTGCCTGTGCCGCACGGTG |
| NC_045512.2_19mer_win1_13500 | 13500 | 13518 | 235 | ACCGTGCGGCACAGGCACT | 1438 | AGTGCCTGTGCCGCACGGT |
| NC_045512.2_19mer_win1_13501 | 13501 | 13519 | 236 | CCGTGCGGCACAGGCACTA | 1439 | TAGTGCCTGTGCCGCACGG |
| NC_045512.2_19mer_win1_13502 | 13502 | 13520 | 237 | CGTGCGGCACAGGCACTAG | 1440 | CTAGTGCCTGTGCCGCACG |
| NC_045512.2_19mer_win1_13762 | 13762 | 13780 | 238 | GGTGACATGGTACCACATA | 1441 | TATGTGGTACCATGTCACC |
| NC_045512.2_19mer_win1_13763 | 13763 | 13781 | 239 | GTGACATGGTACCACATAT | 1442 | ATATGTGGTACCATGTCAC |
| NC_045512.2_19mer_win1_13764 | 13764 | 13782 | 240 | TGACATGGTACCACATATA | 1443 | TATATGTGGTACCATGTCA |
| NC_045512.2_19mer_win1_13765 | 13765 | 13783 | 241 | GACATGGTACCACATATAT | 1444 | ATATATGTGGTACCATGTC |
| NC_045512.2_19mer_win1_13766 | 13766 | 13784 | 242 | ACATGGTACCACATATATC | 1445 | GATATATGTGGTACCATGT |
| NC_045512.2_19mer_win1_13767 | 13767 | 13785 | 243 | CATGGTACCACATATATCA | 1446 | TGATATATGTGGTACCATG |
| NC_045512.2_19mer_win1_13768 | 13768 | 13786 | 244 | ATGGTACCACATATATCAC | 1447 | GTGATATATGTGGTACCAT |
| NC_045512.2_19mer_win1_13769 | 13769 | 13787 | 245 | TGGTACCACATATATCACG | 1448 | CGTGATATATGTGGTACCA |
| NC_045512.2_19mer_win1_13770 | 13770 | 13788 | 246 | GGTACCACATATATCACGT | 1449 | ACGTGATATATGTGGTACC |
| NC_045512.2_19mer_win1_13771 | 13771 | 13789 | 247 | GTACCACATATATCACGTC | 1450 | GACGTGATATATGTGGTAC |
| NC_045512.2_19mer_win1_13772 | 13772 | 13790 | 248 | TACCACATATATCACGTCA | 1451 | TGACGTGATATATGTGGTA |
| NC_045512.2_19mer_win1_14290 | 14290 | 14308 | 249 | GACCGTTATTTTAAATATT | 1452 | AATATTTAAAATAACGGTC |
| NC_045512.2_19mer_win1_14291 | 14291 | 14309 | 250 | ACCGTTATTTTAAATATTG | 1453 | CAATATTTAAAATAACGGT |
| NC_045512.2_19mer_win1_14292 | 14292 | 14310 | 251 | CCGTTATTTTAAATATTGG | 1454 | CCAATATTTAAAATAACGG |
| NC_045512.2_19mer_win1_14293 | 14293 | 14311 | 252 | CGTTATTTTAAATATTGGG | 1455 | CCCAATATTTAAAATAACG |
| NC_045512.2_19mer_win1_14294 | 14294 | 14312 | 253 | GTTATTTTAAATATTGGGA | 1456 | TCCCAATATTTAAAATAAC |
| NC_045512.2_19mer_win1_14404 | 14404 | 14422 | 254 | CCACCTACAAGTTTTGGAC | 1457 | GTCCAAAACTTGTAGGTGG |
| NC_045512.2_19mer_win1_14405 | 14405 | 14423 | 255 | CACCTACAAGTTTTGGACC | 1458 | GGTCCAAAACTTGTAGGTG |
| NC_045512.2_19mer_win1_14406 | 14406 | 14424 | 256 | ACCTACAAGTTTTGGACCA | 1459 | TGGTCCAAAACTTGTAGGT |
| NC_045512.2_19mer_win1_14407 | 14407 | 14425 | 257 | CCTACAAGTTTTGGACCAC | 1460 | GTGGTCCAAAACTTGTAGG |
| NC_045512.2_19mer_win1_14408 | 14408 | 14426 | 258 | CTACAAGTTTTGGACCACT | 1461 | AGTGGTCCAAAACTTGTAG |
| NC_045512.2_19mer_win1_14409 | 14409 | 14427 | 259 | TACAAGTTTTGGACCACTA | 1462 | TAGTGGTCCAAAACTTGTA |
| NC_045512.2_19mer_win1_14410 | 14410 | 14428 | 260 | ACAAGTTTTGGACCACTAG | 1463 | CTAGTGGTCCAAAACTTGT |
| NC_045512.2_19mer_win1_14411 | 14411 | 14429 | 261 | CAAGTTTTGGACCACTAGT | 1464 | ACTAGTGGTCCAAAACTTG |
| NC_045512.2_19mer_win1_14500 | 14500 | 14518 | 262 | GTACATAATCAGGATGTAA | 1465 | TTACATCCTGATTATGTAC |
| NC_045512.2_19mer_win1_14501 | 14501 | 14519 | 263 | TACATAATCAGGATGTAAA | 1466 | TTTACATCCTGATTATGTA |
| NC_045512.2_19mer_win1_14502 | 14502 | 14520 | 264 | ACATAATCAGGATGTAAAC | 1467 | GTTTACATCCTGATTATGT |
| NC_045512.2_19mer_win1_14503 | 14503 | 14521 | 265 | CATAATCAGGATGTAAACT | 1468 | AGTTTACATCCTGATTATG |
| NC_045512.2_19mer_win1_14504 | 14504 | 14522 | 266 | ATAATCAGGATGTAAACTT | 1469 | AAGTTTACATCCTGATTAT |
| NC_045512.2_19mer_win1_14505 | 14505 | 14523 | 267 | TAATCAGGATGTAAACTTA | 1470 | TAAGTTTACATCCTGATTA |
| NC_045512.2_19mer_win1_14506 | 14506 | 14524 | 268 | AATCAGGATGTAAACTTAC | 1471 | GTAAGTTTACATCCTGATT |
| NC_045512.2_19mer_win1_14507 | 14507 | 14525 | 269 | ATCAGGATGTAAACTTACA | 1472 | TGTAAGTTTACATCCTGAT |
| NC_045512.2_19mer_win1_14508 | 14508 | 14526 | 270 | TCAGGATGTAAACTTACAT | 1473 | ATGTAAGTTTACATCCTGA |
| NC_045512.2_19mer_win1_14509 | 14509 | 14527 | 271 | CAGGATGTAAACTTACATA | 1474 | TATGTAAGTTTACATCCTG |
| NC_045512.2_19mer_win1_14510 | 14510 | 14528 | 272 | AGGATGTAAACTTACATAG | 1475 | CTATGTAAGTTTACATCCT |
| NC_045512.2_19mer_win1_14511 | 14511 | 14529 | 273 | GGATGTAAACTTACATAGC | 1476 | GCTATGTAAGTTTACATCC |
| NC_045512.2_19mer_win1_14512 | 14512 | 14530 | 274 | GATGTAAACTTACATAGCT | 1477 | AGCTATGTAAGTTTACATC |
| NC_045512.2_19mer_win1_14513 | 14513 | 14531 | 275 | ATGTAAACTTACATAGCTC | 1478 | GAGCTATGTAAGTTTACAT |
| NC_045512.2_19mer_win1_14623 | 14623 | 14641 | 276 | TGCTTTTCAGTAGCTGCAC | 1479 | GTGCAGCTACTGAAAAGCA |
| NC_045512.2_19mer_win1_14624 | 14624 | 14642 | 277 | GCTTTTCAGTAGCTGCACT | 1480 | AGTGCAGCTACTGAAAAGC |
| NC_045512.2_19mer_win1_14650 | 14650 | 14668 | 278 | AATGTTGCTTTTCAAACTG | 1481 | CAGTTTGAAAAGCAACATT |
| NC_045512.2_19mer_win1_14651 | 14651 | 14669 | 279 | ATGTTGCTTTTCAAACTGT | 1482 | ACAGTTTGAAAAGCAACAT |
| NC_045512.2_19mer_win1_14652 | 14652 | 14670 | 280 | TGTTGCTTTTCAAACTGTC | 1483 | GACAGTTTGAAAAGCAACA |
| NC_045512.2_19mer_win1_14653 | 14653 | 14671 | 281 | GTTGCTTTTCAAACTGTCA | 1484 | TGACAGTTTGAAAAGCAAC |
| NC_045512.2_19mer_win1_14654 | 14654 | 14672 | 282 | TTGCTTTTCAAACTGTCAA | 1485 | TTGACAGTTTGAAAAGCAA |
| NC_045512.2_19mer_win1_14655 | 14655 | 14673 | 283 | TGCTTTTCAAACTGTCAAA | 1486 | TTTGACAGTTTGAAAAGCA |
| NC_045512.2_19mer_win1_14656 | 14656 | 14674 | 284 | GCTTTTCAAACTGTCAAAC | 1487 | GTTTGACAGTTTGAAAAGC |
| NC_045512.2_19mer_win1_14657 | 14657 | 14675 | 285 | CTTTTCAAACTGTCAAACC | 1488 | GGTTTGACAGTTTGAAAAG |
| NC_045512.2_19mer_win1_14658 | 14658 | 14676 | 286 | TTTTCAAACTGTCAAACCC | 1489 | GGGTTTGACAGTTTGAAAA |
| NC_045512.2_19mer_win1_14659 | 14659 | 14677 | 287 | TTTCAAACTGTCAAACCCG | 1490 | CGGGTTTGACAGTTTGAAA |
| NC_045512.2_19mer_win1_14660 | 14660 | 14678 | 288 | TTCAAACTGTCAAACCCGG | 1491 | CCGGGTTTGACAGTTTGAA |
| NC_045512.2_19mer_win1_14661 | 14661 | 14679 | 289 | TCAAACTGTCAAACCCGGT | 1492 | ACCGGGTTTGACAGTTTGA |
| NC_045512.2_19mer_win1_14662 | 14662 | 14680 | 290 | CAAACTGTCAAACCCGGTA | 1493 | TACCGGGTTTGACAGTTTG |
| NC_045512.2_19mer_win1_14663 | 14663 | 14681 | 291 | AAACTGTCAAACCCGGTAA | 1494 | TTACCGGGTTTGACAGTTT |
| NC_045512.2_19mer_win1_14664 | 14664 | 14682 | 292 | AACTGTCAAACCCGGTAAT | 1495 | ATTACCGGGTTTGACAGTT |
| NC_045512.2_19mer_win1_14665 | 14665 | 14683 | 293 | ACTGTCAAACCCGGTAATT | 1496 | AATTACCGGGTTTGACAGT |
| NC_045512.2_19mer_win1_14666 | 14666 | 14684 | 294 | CTGTCAAACCCGGTAATTT | 1497 | AAATTACCGGGTTTGACAG |
| NC_045512.2_19mer_win1_14667 | 14667 | 14685 | 295 | TGTCAAACCCGGTAATTTT | 1498 | AAAATTACCGGGTTTGACA |
| NC_045512.2_19mer_win1_14668 | 14668 | 14686 | 296 | GTCAAACCCGGTAATTTTA | 1499 | TAAAATTACCGGGTTTGAC |
| NC_045512.2_19mer_win1_14669 | 14669 | 14687 | 297 | TCAAACCCGGTAATTTTAA | 1500 | TTAAAATTACCGGGTTTGA |
| NC_045512.2_19mer_win1_14698 | 14698 | 14716 | 298 | TATGACTTTGCTGTGTCTA | 1501 | TAGACACAGCAAAGTCATA |
| NC_045512.2_19mer_win1_14699 | 14699 | 14717 | 299 | ATGACTTTGCTGTGTCTAA | 1502 | TTAGACACAGCAAAGTCAT |
| NC_045512.2_19mer_win1_14722 | 14722 | 14740 | 300 | TTCTTTAAGGAAGGAAGTT | 1503 | AACTTCCTTCCTTAAAGAA |
| NC_045512.2_19mer_win1_14723 | 14723 | 14741 | 301 | TCTTTAAGGAAGGAAGTTC | 1504 | GAACTTCCTTCCTTAAAGA |
| NC_045512.2_19mer_win1_14724 | 14724 | 14742 | 302 | CTTTAAGGAAGGAAGTTCT | 1505 | AGAACTTCCTTCCTTAAAG |
| NC_045512.2_19mer_win1_14725 | 14725 | 14743 | 303 | TTTAAGGAAGGAAGTTCTG | 1506 | CAGAACTTCCTTCCTTAAA |
| NC_045512.2_19mer_win1_14726 | 14726 | 14744 | 304 | TTAAGGAAGGAAGTTCTGT | 1507 | ACAGAACTTCCTTCCTTAA |
| NC_045512.2_19mer_win1_14727 | 14727 | 14745 | 305 | TAAGGAAGGAAGTTCTGTT | 1508 | AACAGAACTTCCTTCCTTA |
| NC_045512.2_19mer_win1_14728 | 14728 | 14746 | 306 | AAGGAAGGAAGTTCTGTTG | 1509 | CAACAGAACTTCCTTCCTT |
| NC_045512.2_19mer_win1_14729 | 14729 | 14747 | 307 | AGGAAGGAAGTTCTGTTGA | 1510 | TCAACAGAACTTCCTTCCT |
| NC_045512.2_19mer_win1_14730 | 14730 | 14748 | 308 | GGAAGGAAGTTCTGTTGAA | 1511 | TTCAACAGAACTTCCTTCC |
| NC_045512.2_19mer_win1_14750 | 14750 | 14768 | 309 | TAAAACACTTCTTCTTTGC | 1512 | GCAAAGAAGAAGTGTTTTA |
| NC_045512.2_19mer_win1_14751 | 14751 | 14769 | 310 | AAAACACTTCTTCTTTGCT | 1513 | AGCAAAGAAGAAGTGTTTT |
| NC_045512.2_19mer_win1_14752 | 14752 | 14770 | 311 | AAACACTTCTTCTTTGCTC | 1514 | GAGCAAAGAAGAAGTGTTT |
| NC_045512.2_19mer_win1_14753 | 14753 | 14771 | 312 | AACACTTCTTCTTTGCTCA | 1515 | TGAGCAAAGAAGAAGTGTT |
| NC_045512.2_19mer_win1_14754 | 14754 | 14772 | 313 | ACACTTCTTCTTTGCTCAG | 1516 | CTGAGCAAAGAAGAAGTGT |
| NC_045512.2_19mer_win1_14755 | 14755 | 14773 | 314 | CACTTCTTCTTTGCTCAGG | 1517 | CCTGAGCAAAGAAGAAGTG |
| NC_045512.2_19mer_win1_14756 | 14756 | 14774 | 315 | ACTTCTTCTTTGCTCAGGA | 1518 | TCCTGAGCAAAGAAGAAGT |
| NC_045512.2_19mer_win1_14757 | 14757 | 14775 | 316 | CTTCTTCTTTGCTCAGGAT | 1519 | ATCCTGAGCAAAGAAGAAG |
| NC_045512.2_19mer_win1_14758 | 14758 | 14776 | 317 | TTCTTCTTTGCTCAGGATG | 1520 | CATCCTGAGCAAAGAAGAA |
| NC_045512.2_19mer_win1_14759 | 14759 | 14777 | 318 | TCTTCTTTGCTCAGGATGG | 1521 | CCATCCTGAGCAAAGAAGA |
| NC_045512.2_19mer_win1_14821 | 14821 | 14839 | 319 | CCAACAATGTGTGATATCA | 1522 | TGATATCACACATTGTTGG |
| NC_045512.2_19mer_win1_14822 | 14822 | 14840 | 320 | CAACAATGTGTGATATCAG | 1523 | CTGATATCACACATTGTTG |
| NC_045512.2_19mer_win1_14823 | 14823 | 14841 | 321 | AACAATGTGTGATATCAGA | 1524 | TCTGATATCACACATTGTT |
| NC_045512.2_19mer_win1_14824 | 14824 | 14842 | 322 | ACAATGTGTGATATCAGAC | 1525 | GTCTGATATCACACATTGT |
| NC_045512.2_19mer_win1_14825 | 14825 | 14843 | 323 | CAATGTGTGATATCAGACA | 1526 | TGTCTGATATCACACATTG |
| NC_045512.2_19mer_win1_14826 | 14826 | 14844 | 324 | AATGTGTGATATCAGACAA | 1527 | TTGTCTGATATCACACATT |
| NC_045512.2_19mer_win1_14827 | 14827 | 14845 | 325 | ATGTGTGATATCAGACAAC | 1528 | GTTGTCTGATATCACACAT |
| NC_045512.2_19mer_win1_14828 | 14828 | 14846 | 326 | TGTGTGATATCAGACAACT | 1529 | AGTTGTCTGATATCACACA |
| NC_045512.2_19mer_win1_14854 | 14854 | 14872 | 327 | GTAGTTGAAGTTGTTGATA | 1530 | TATCAACAACTTCAACTAC |
| NC_045512.2_19mer_win1_14855 | 14855 | 14873 | 328 | TAGTTGAAGTTGTTGATAA | 1531 | TTATCAACAACTTCAACTA |
| NC_045512.2_19mer_win1_14875 | 14875 | 14893 | 329 | TACTTTGATTGTTACGATG | 1532 | CATCGTAACAATCAAAGTA |
| NC_045512.2_19mer_win1_14876 | 14876 | 14894 | 330 | ACTTTGATTGTTACGATGG | 1533 | CCATCGTAACAATCAAAGT |
| NC_045512.2_19mer_win1_14877 | 14877 | 14895 | 331 | CTTTGATTGTTACGATGGT | 1534 | ACCATCGTAACAATCAAAG |
| NC_045512.2_19mer_win1_14878 | 14878 | 14896 | 332 | TTTGATTGTTACGATGGTG | 1535 | CACCATCGTAACAATCAAA |
| NC_045512.2_19mer_win1_14879 | 14879 | 14897 | 333 | TTGATTGTTACGATGGTGG | 1536 | CCACCATCGTAACAATCAA |
| NC_045512.2_19mer_win1_14880 | 14880 | 14898 | 334 | TGATTGTTACGATGGTGGC | 1537 | GCCACCATCGTAACAATCA |
| NC_045512.2_19mer_win1_14881 | 14881 | 14899 | 335 | GATTGTTACGATGGTGGCT | 1538 | AGCCACCATCGTAACAATC |
| NC_045512.2_19mer_win1_14882 | 14882 | 14900 | 336 | ATTGTTACGATGGTGGCTG | 1539 | CAGCCACCATCGTAACAAT |
| NC_045512.2_19mer_win1_14883 | 14883 | 14901 | 337 | TTGTTACGATGGTGGCTGT | 1540 | ACAGCCACCATCGTAACAA |
| NC_045512.2_19mer_win1_14884 | 14884 | 14902 | 338 | TGTTACGATGGTGGCTGTA | 1541 | TACAGCCACCATCGTAACA |
| NC_045512.2_19mer_win1_14885 | 14885 | 14903 | 339 | GTTACGATGGTGGCTGTAT | 1542 | ATACAGCCACCATCGTAAC |
| NC_045512.2_19mer_win1_14962 | 14962 | 14980 | 340 | AAATGGGGTAAGGCTAGAC | 1543 | GTCTAGCCTTACCCCATTT |
| NC_045512.2_19mer_win1_14963 | 14963 | 14981 | 341 | AATGGGGTAAGGCTAGACT | 1544 | AGTCTAGCCTTACCCCATT |
| NC_045512.2_19mer_win1_14964 | 14964 | 14982 | 342 | ATGGGGTAAGGCTAGACTT | 1545 | AAGTCTAGCCTTACCCCAT |
| NC_045512.2_19mer_win1_14965 | 14965 | 14983 | 343 | TGGGGTAAGGCTAGACTTT | 1546 | AAAGTCTAGCCTTACCCCA |
| NC_045512.2_19mer_win1_14966 | 14966 | 14984 | 344 | GGGGTAAGGCTAGACTTTA | 1547 | TAAAGTCTAGCCTTACCCC |
| NC_045512.2_19mer_win1_14967 | 14967 | 14985 | 345 | GGGTAAGGCTAGACTTTAT | 1548 | ATAAAGTCTAGCCTTACCC |
| NC_045512.2_19mer_win1_14968 | 14968 | 14986 | 346 | GGTAAGGCTAGACTTTATT | 1549 | AATAAAGTCTAGCCTTACC |
| NC_045512.2_19mer_win1_14969 | 14969 | 14987 | 347 | GTAAGGCTAGACTTTATTA | 1550 | TAATAAAGTCTAGCCTTAC |
| NC_045512.2_19mer_win1_14970 | 14970 | 14988 | 348 | TAAGGCTAGACTTTATTAT | 1551 | ATAATAAAGTCTAGCCTTA |
| NC_045512.2_19mer_win1_14971 | 14971 | 14989 | 349 | AAGGCTAGACTTTATTATG | 1552 | CATAATAAAGTCTAGCCTT |
| NC_045512.2_19mer_win1_14972 | 14972 | 14990 | 350 | AGGCTAGACTTTATTATGA | 1553 | TCATAATAAAGTCTAGCCT |
| NC_045512.2_19mer_win1_14992 | 14992 | 15010 | 351 | TCAATGAGTTATGAGGATC | 1554 | GATCCTCATAACTCATTGA |
| NC_045512.2_19mer_win1_14993 | 14993 | 15011 | 352 | CAATGAGTTATGAGGATCA | 1555 | TGATCCTCATAACTCATTG |
| NC_045512.2_19mer_win1_14994 | 14994 | 15012 | 353 | AATGAGTTATGAGGATCAA | 1556 | TTGATCCTCATAACTCATT |
| NC_045512.2_19mer_win1_14995 | 14995 | 15013 | 354 | ATGAGTTATGAGGATCAAG | 1557 | CTTGATCCTCATAACTCAT |
| NC_045512.2_19mer_win1_14996 | 14996 | 15014 | 355 | TGAGTTATGAGGATCAAGA | 1558 | TCTTGATCCTCATAACTCA |
| NC_045512.2_19mer_win1_14997 | 14997 | 15015 | 356 | GAGTTATGAGGATCAAGAT | 1559 | ATCTTGATCCTCATAACTC |
| NC_045512.2_19mer_win1_14998 | 14998 | 15016 | 357 | AGTTATGAGGATCAAGATG | 1560 | CATCTTGATCCTCATAACT |
| NC_045512.2_19mer_win1_14999 | 14999 | 15017 | 358 | GTTATGAGGATCAAGATGC | 1561 | GCATCTTGATCCTCATAAC |
| NC_045512.2_19mer_win1_15000 | 15000 | 15018 | 359 | TTATGAGGATCAAGATGCA | 1562 | TGCATCTTGATCCTCATAA |
| NC_045512.2_19mer_win1_15001 | 15001 | 15019 | 360 | TATGAGGATCAAGATGCAC | 1563 | GTGCATCTTGATCCTCATA |
| NC_045512.2_19mer_win1_15002 | 15002 | 15020 | 361 | ATGAGGATCAAGATGCACT | 1564 | AGTGCATCTTGATCCTCAT |
| NC_045512.2_19mer_win1_15055 | 15055 | 15073 | 362 | ATAACTCAAATGAATCTTA | 1565 | TAAGATTCATTTGAGTTAT |
| NC_045512.2_19mer_win1_15056 | 15056 | 15074 | 363 | TAACTCAAATGAATCTTAA | 1566 | TTAAGATTCATTTGAGTTA |
| NC_045512.2_19mer_win1_15057 | 15057 | 15075 | 364 | AACTCAAATGAATCTTAAG | 1567 | CTTAAGATTCATTTGAGTT |
| NC_045512.2_19mer_win1_15058 | 15058 | 15076 | 365 | ACTCAAATGAATCTTAAGT | 1568 | ACTTAAGATTCATTTGAGT |
| NC_045512.2_19mer_win1_15059 | 15059 | 15077 | 366 | CTCAAATGAATCTTAAGTA | 1569 | TACTTAAGATTCATTTGAG |
| NC_045512.2_19mer_win1_15060 | 15060 | 15078 | 367 | TCAAATGAATCTTAAGTAT | 1570 | ATACTTAAGATTCATTTGA |
| NC_045512.2_19mer_win1_15061 | 15061 | 15079 | 368 | CAAATGAATCTTAAGTATG | 1571 | CATACTTAAGATTCATTTG |
| NC_045512.2_19mer_win1_15062 | 15062 | 15080 | 369 | AAATGAATCTTAAGTATGC | 1572 | GCATACTTAAGATTCATTT |
| NC_045512.2_19mer_win1_15063 | 15063 | 15081 | 370 | AATGAATCTTAAGTATGCC | 1573 | GGCATACTTAAGATTCATT |
| NC_045512.2_19mer_win1_15064 | 15064 | 15082 | 371 | ATGAATCTTAAGTATGCCA | 1574 | TGGCATACTTAAGATTCAT |
| NC_045512.2_19mer_win1_15065 | 15065 | 15083 | 372 | TGAATCTTAAGTATGCCAT | 1575 | ATGGCATACTTAAGATTCA |
| NC_045512.2_19mer_win1_15066 | 15066 | 15084 | 373 | GAATCTTAAGTATGCCATT | 1576 | AATGGCATACTTAAGATTC |
| NC_045512.2_19mer_win1_15067 | 15067 | 15085 | 374 | AATCTTAAGTATGCCATTA | 1577 | TAATGGCATACTTAAGATT |
| NC_045512.2_19mer_win1_15068 | 15068 | 15086 | 375 | ATCTTAAGTATGCCATTAG | 1578 | CTAATGGCATACTTAAGAT |
| NC_045512.2_19mer_win1_15069 | 15069 | 15087 | 376 | TCTTAAGTATGCCATTAGT | 1579 | ACTAATGGCATACTTAAGA |
| NC_045512.2_19mer_win1_15070 | 15070 | 15088 | 377 | CTTAAGTATGCCATTAGTG | 1580 | CACTAATGGCATACTTAAG |
| NC_045512.2_19mer_win1_15071 | 15071 | 15089 | 378 | TTAAGTATGCCATTAGTGC | 1581 | GCACTAATGGCATACTTAA |
| NC_045512.2_19mer_win1_15072 | 15072 | 15090 | 379 | TAAGTATGCCATTAGTGCA | 1582 | TGCACTAATGGCATACTTA |
| NC_045512.2_19mer_win1_15073 | 15073 | 15091 | 380 | AAGTATGCCATTAGTGCAA | 1583 | TTGCACTAATGGCATACTT |
| NC_045512.2_19mer_win1_15074 | 15074 | 15092 | 381 | AGTATGCCATTAGTGCAAA | 1584 | TTTGCACTAATGGCATACT |
| NC_045512.2_19mer_win1_15075 | 15075 | 15093 | 382 | GTATGCCATTAGTGCAAAG | 1585 | CTTTGCACTAATGGCATAC |
| NC_045512.2_19mer_win1_15076 | 15076 | 15094 | 383 | TATGCCATTAGTGCAAAGA | 1586 | TCTTTGCACTAATGGCATA |
| NC_045512.2_19mer_win1_15077 | 15077 | 15095 | 384 | ATGCCATTAGTGCAAAGAA | 1587 | TTCTTTGCACTAATGGCAT |
| NC_045512.2_19mer_win1_15078 | 15078 | 15096 | 385 | TGCCATTAGTGCAAAGAAT | 1588 | ATTCTTTGCACTAATGGCA |
| NC_045512.2_19mer_win1_15079 | 15079 | 15097 | 386 | GCCATTAGTGCAAAGAATA | 1589 | TATTCTTTGCACTAATGGC |
| NC_045512.2_19mer_win1_15080 | 15080 | 15098 | 387 | CCATTAGTGCAAAGAATAG | 1590 | CTATTCTTTGCACTAATGG |
| NC_045512.2_19mer_win1_15081 | 15081 | 15099 | 388 | CATTAGTGCAAAGAATAGA | 1591 | TCTATTCTTTGCACTAATG |
| NC_045512.2_19mer_win1_15082 | 15082 | 15100 | 389 | ATTAGTGCAAAGAATAGAG | 1592 | CTCTATTCTTTGCACTAAT |
| NC_045512.2_19mer_win1_15083 | 15083 | 15101 | 390 | TTAGTGCAAAGAATAGAGC | 1593 | GCTCTATTCTTTGCACTAA |
| NC_045512.2_19mer_win1_15084 | 15084 | 15102 | 391 | TAGTGCAAAGAATAGAGCT | 1594 | AGCTCTATTCTTTGCACTA |
| NC_045512.2_19mer_win1_15085 | 15085 | 15103 | 392 | AGTGCAAAGAATAGAGCTC | 1595 | GAGCTCTATTCTTTGCACT |
| NC_045512.2_19mer_win1_15086 | 15086 | 15104 | 393 | GTGCAAAGAATAGAGCTCG | 1596 | CGAGCTCTATTCTTTGCAC |
| NC_045512.2_19mer_win1_15087 | 15087 | 15105 | 394 | TGCAAAGAATAGAGCTCGC | 1597 | GCGAGCTCTATTCTTTGCA |
| NC_045512.2_19mer_win1_15088 | 15088 | 15106 | 395 | GCAAAGAATAGAGCTCGCA | 1598 | TGCGAGCTCTATTCTTTGC |
| NC_045512.2_19mer_win1_15089 | 15089 | 15107 | 396 | CAAAGAATAGAGCTCGCAC | 1599 | GTGCGAGCTCTATTCTTTG |
| NC_045512.2_19mer_win1_15090 | 15090 | 15108 | 397 | AAAGAATAGAGCTCGCACC | 1600 | GGTGCGAGCTCTATTCTTT |
| NC_045512.2_19mer_win1_15091 | 15091 | 15109 | 398 | AAGAATAGAGCTCGCACCG | 1601 | CGGTGCGAGCTCTATTCTT |
| NC_045512.2_19mer_win1_15092 | 15092 | 15110 | 399 | AGAATAGAGCTCGCACCGT | 1602 | ACGGTGCGAGCTCTATTCT |
| NC_045512.2_19mer_win1_15093 | 15093 | 15111 | 400 | GAATAGAGCTCGCACCGTA | 1603 | TACGGTGCGAGCTCTATTC |
| NC_045512.2_19mer_win1_15094 | 15094 | 15112 | 401 | AATAGAGCTCGCACCGTAG | 1604 | CTACGGTGCGAGCTCTATT |
| NC_045512.2_19mer_win1_15095 | 15095 | 15113 | 402 | ATAGAGCTCGCACCGTAGC | 1605 | GCTACGGTGCGAGCTCTAT |
| NC_045512.2_19mer_win1_15096 | 15096 | 15114 | 403 | TAGAGCTCGCACCGTAGCT | 1606 | AGCTACGGTGCGAGCTCTA |
| NC_045512.2_19mer_win1_15097 | 15097 | 15115 | 404 | AGAGCTCGCACCGTAGCTG | 1607 | CAGCTACGGTGCGAGCTCT |
| NC_045512.2_19mer_win1_15098 | 15098 | 15116 | 405 | GAGCTCGCACCGTAGCTGG | 1608 | CCAGCTACGGTGCGAGCTC |
| NC_045512.2_19mer_win1_15099 | 15099 | 15117 | 406 | AGCTCGCACCGTAGCTGGT | 1609 | ACCAGCTACGGTGCGAGCT |
| NC_045512.2_19mer_win1_15100 | 15100 | 15118 | 407 | GCTCGCACCGTAGCTGGTG | 1610 | CACCAGCTACGGTGCGAGC |
| NC_045512.2_19mer_win1_15101 | 15101 | 15119 | 408 | CTCGCACCGTAGCTGGTGT | 1611 | ACACCAGCTACGGTGCGAG |
| NC_045512.2_19mer_win1_15102 | 15102 | 15120 | 409 | TCGCACCGTAGCTGGTGTC | 1612 | GACACCAGCTACGGTGCGA |
| NC_045512.2_19mer_win1_15103 | 15103 | 15121 | 410 | CGCACCGTAGCTGGTGTCT | 1613 | AGACACCAGCTACGGTGCG |
| NC_045512.2_19mer_win1_15104 | 15104 | 15122 | 411 | GCACCGTAGCTGGTGTCTC | 1614 | GAGACACCAGCTACGGTGC |
| NC_045512.2_19mer_win1_15105 | 15105 | 15123 | 412 | CACCGTAGCTGGTGTCTCT | 1615 | AGAGACACCAGCTACGGTG |
| NC_045512.2_19mer_win1_15106 | 15106 | 15124 | 413 | ACCGTAGCTGGTGTCTCTA | 1616 | TAGAGACACCAGCTACGGT |
| NC_045512.2_19mer_win1_15107 | 15107 | 15125 | 414 | CCGTAGCTGGTGTCTCTAT | 1617 | ATAGAGACACCAGCTACGG |
| NC_045512.2_19mer_win1_15108 | 15108 | 15126 | 415 | CGTAGCTGGTGTCTCTATC | 1618 | GATAGAGACACCAGCTACG |
| NC_045512.2_19mer_win1_15109 | 15109 | 15127 | 416 | GTAGCTGGTGTCTCTATCT | 1619 | AGATAGAGACACCAGCTAC |
| NC_045512.2_19mer_win1_15110 | 15110 | 15128 | 417 | TAGCTGGTGTCTCTATCTG | 1620 | CAGATAGAGACACCAGCTA |
| NC_045512.2_19mer_win1_15111 | 15111 | 15129 | 418 | AGCTGGTGTCTCTATCTGT | 1621 | ACAGATAGAGACACCAGCT |
| NC_045512.2_19mer_win1_15112 | 15112 | 15130 | 419 | GCTGGTGTCTCTATCTGTA | 1622 | TACAGATAGAGACACCAGC |
| NC_045512.2_19mer_win1_15113 | 15113 | 15131 | 420 | CTGGTGTCTCTATCTGTAG | 1623 | CTACAGATAGAGACACCAG |
| NC_045512.2_19mer_win1_15114 | 15114 | 15132 | 421 | TGGTGTCTCTATCTGTAGT | 1624 | ACTACAGATAGAGACACCA |
| NC_045512.2_19mer_win1_15115 | 15115 | 15133 | 422 | GGTGTCTCTATCTGTAGTA | 1625 | TACTACAGATAGAGACACC |
| NC_045512.2_19mer_win1_15116 | 15116 | 15134 | 423 | GTGTCTCTATCTGTAGTAC | 1626 | GTACTACAGATAGAGACAC |
| NC_045512.2_19mer_win1_15117 | 15117 | 15135 | 424 | TGTCTCTATCTGTAGTACT | 1627 | AGTACTACAGATAGAGACA |
| NC_045512.2_19mer_win1_15118 | 15118 | 15136 | 425 | GTCTCTATCTGTAGTACTA | 1628 | TAGTACTACAGATAGAGAC |
| NC_045512.2_19mer_win1_15119 | 15119 | 15137 | 426 | TCTCTATCTGTAGTACTAT | 1629 | ATAGTACTACAGATAGAGA |
| NC_045512.2_19mer_win1_15120 | 15120 | 15138 | 427 | CTCTATCTGTAGTACTATG | 1630 | CATAGTACTACAGATAGAG |
| NC_045512.2_19mer_win1_15121 | 15121 | 15139 | 428 | TCTATCTGTAGTACTATGA | 1631 | TCATAGTACTACAGATAGA |
| NC_045512.2_19mer_win1_15122 | 15122 | 15140 | 429 | CTATCTGTAGTACTATGAC | 1632 | GTCATAGTACTACAGATAG |
| NC_045512.2_19mer_win1_15172 | 15172 | 15190 | 430 | TCAATAGCCGCCACTAGAG | 1633 | CTCTAGTGGCGGCTATTGA |
| NC_045512.2_19mer_win1_15173 | 15173 | 15191 | 431 | CAATAGCCGCCACTAGAGG | 1634 | CCTCTAGTGGCGGCTATTG |
| NC_045512.2_19mer_win1_15174 | 15174 | 15192 | 432 | AATAGCCGCCACTAGAGGA | 1635 | TCCTCTAGTGGCGGCTATT |
| NC_045512.2_19mer_win1_15175 | 15175 | 15193 | 433 | ATAGCCGCCACTAGAGGAG | 1636 | CTCCTCTAGTGGCGGCTAT |
| NC_045512.2_19mer_win1_15176 | 15176 | 15194 | 434 | TAGCCGCCACTAGAGGAGC | 1637 | GCTCCTCTAGTGGCGGCTA |
| NC_045512.2_19mer_win1_15177 | 15177 | 15195 | 435 | AGCCGCCACTAGAGGAGCT | 1638 | AGCTCCTCTAGTGGCGGCT |
| NC_045512.2_19mer_win1_15178 | 15178 | 15196 | 436 | GCCGCCACTAGAGGAGCTA | 1639 | TAGCTCCTCTAGTGGCGGC |
| NC_045512.2_19mer_win1_15179 | 15179 | 15197 | 437 | CCGCCACTAGAGGAGCTAC | 1640 | GTAGCTCCTCTAGTGGCGG |
| NC_045512.2_19mer_win1_15180 | 15180 | 15198 | 438 | CGCCACTAGAGGAGCTACT | 1641 | AGTAGCTCCTCTAGTGGCG |
| NC_045512.2_19mer_win1_15181 | 15181 | 15199 | 439 | GCCACTAGAGGAGCTACTG | 1642 | CAGTAGCTCCTCTAGTGGC |
| NC_045512.2_19mer_win1_15182 | 15182 | 15200 | 440 | CCACTAGAGGAGCTACTGT | 1643 | ACAGTAGCTCCTCTAGTGG |
| NC_045512.2_19mer_win1_15310 | 15310 | 15328 | 441 | AGAGCCATGCCTAACATGC | 1644 | GCATGTTAGGCATGGCTCT |
| NC_045512.2_19mer_win1_15311 | 15311 | 15329 | 442 | GAGCCATGCCTAACATGCT | 1645 | AGCATGTTAGGCATGGCTC |
| NC_045512.2_19mer_win1_15312 | 15312 | 15330 | 443 | AGCCATGCCTAACATGCTT | 1646 | AAGCATGTTAGGCATGGCT |
| NC_045512.2_19mer_win1_15313 | 15313 | 15331 | 444 | GCCATGCCTAACATGCTTA | 1647 | TAAGCATGTTAGGCATGGC |
| NC_045512.2_19mer_win1_15314 | 15314 | 15332 | 445 | CCATGCCTAACATGCTTAG | 1648 | CTAAGCATGTTAGGCATGG |
| NC_045512.2_19mer_win1_15346 | 15346 | 15364 | 446 | CTTGTTCTTGCTCGCAAAC | 1649 | GTTTGCGAGCAAGAACAAG |
| NC_045512.2_19mer_win1_15347 | 15347 | 15365 | 447 | TTGTTCTTGCTCGCAAACA | 1650 | TGTTTGCGAGCAAGAACAA |
| NC_045512.2_19mer_win1_15348 | 15348 | 15366 | 448 | TGTTCTTGCTCGCAAACAT | 1651 | ATGTTTGCGAGCAAGAACA |
| NC_045512.2_19mer_win1_15349 | 15349 | 15367 | 449 | GTTCTTGCTCGCAAACATA | 1652 | TATGTTTGCGAGCAAGAAC |
| NC_045512.2_19mer_win1_15496 | 15496 | 15514 | 450 | ACAACTGCTTATGCTAATA | 1653 | TATTAGCATAAGCAGTTGT |
| NC_045512.2_19mer_win1_15497 | 15497 | 15515 | 451 | CAACTGCTTATGCTAATAG | 1654 | CTATTAGCATAAGCAGTTG |
| NC_045512.2_19mer_win1_15498 | 15498 | 15516 | 452 | AACTGCTTATGCTAATAGT | 1655 | ACTATTAGCATAAGCAGTT |
| NC_045512.2_19mer_win1_15499 | 15499 | 15517 | 453 | ACTGCTTATGCTAATAGTG | 1656 | CACTATTAGCATAAGCAGT |
| NC_045512.2_19mer_win1_15500 | 15500 | 15518 | 454 | CTGCTTATGCTAATAGTGT | 1657 | ACACTATTAGCATAAGCAG |
| NC_045512.2_19mer_win1_15622 | 15622 | 15640 | 455 | TATGAGTGTCTCTATAGAA | 1658 | TTCTATAGAGACACTCATA |
| NC_045512.2_19mer_win1_15623 | 15623 | 15641 | 456 | ATGAGTGTCTCTATAGAAA | 1659 | TTTCTATAGAGACACTCAT |
| NC_045512.2_19mer_win1_15624 | 15624 | 15642 | 457 | TGAGTGTCTCTATAGAAAT | 1660 | ATTTCTATAGAGACACTCA |
| NC_045512.2_19mer_win1_15625 | 15625 | 15643 | 458 | GAGTGTCTCTATAGAAATA | 1661 | TATTTCTATAGAGACACTC |
| NC_045512.2_19mer_win1_15626 | 15626 | 15644 | 459 | AGTGTCTCTATAGAAATAG | 1662 | CTATTTCTATAGAGACACT |
| NC_045512.2_19mer_win1_15838 | 15838 | 15856 | 460 | TGGACTGAGACTGACCTTA | 1663 | TAAGGTCAGTCTCAGTCCA |
| NC_045512.2_19mer_win1_15839 | 15839 | 15857 | 461 | GGACTGAGACTGACCTTAC | 1664 | GTAAGGTCAGTCTCAGTCC |
| NC_045512.2_19mer_win1_15840 | 15840 | 15858 | 462 | GACTGAGACTGACCTTACT | 1665 | AGTAAGGTCAGTCTCAGTC |
| NC_045512.2_19mer_win1_15841 | 15841 | 15859 | 463 | ACTGAGACTGACCTTACTA | 1666 | TAGTAAGGTCAGTCTCAGT |
| NC_045512.2_19mer_win1_15842 | 15842 | 15860 | 464 | CTGAGACTGACCTTACTAA | 1667 | TTAGTAAGGTCAGTCTCAG |
| NC_045512.2_19mer_win1_15843 | 15843 | 15861 | 465 | TGAGACTGACCTTACTAAA | 1668 | TTTAGTAAGGTCAGTCTCA |
| NC_045512.2_19mer_win1_15844 | 15844 | 15862 | 466 | GAGACTGACCTTACTAAAG | 1669 | CTTTAGTAAGGTCAGTCTC |
| NC_045512.2_19mer_win1_15845 | 15845 | 15863 | 467 | AGACTGACCTTACTAAAGG | 1670 | CCTTTAGTAAGGTCAGTCT |
| NC_045512.2_19mer_win1_15846 | 15846 | 15864 | 468 | GACTGACCTTACTAAAGGA | 1671 | TCCTTTAGTAAGGTCAGTC |
| NC_045512.2_19mer_win1_15847 | 15847 | 15865 | 469 | ACTGACCTTACTAAAGGAC | 1672 | GTCCTTTAGTAAGGTCAGT |
| NC_045512.2_19mer_win1_15848 | 15848 | 15866 | 470 | CTGACCTTACTAAAGGACC | 1673 | GGTCCTTTAGTAAGGTCAG |
| NC_045512.2_19mer_win1_15849 | 15849 | 15867 | 471 | TGACCTTACTAAAGGACCT | 1674 | AGGTCCTTTAGTAAGGTCA |
| NC_045512.2_19mer_win1_15850 | 15850 | 15868 | 472 | GACCTTACTAAAGGACCTC | 1675 | GAGGTCCTTTAGTAAGGTC |
| NC_045512.2_19mer_win1_15851 | 15851 | 15869 | 473 | ACCTTACTAAAGGACCTCA | 1676 | TGAGGTCCTTTAGTAAGGT |
| NC_045512.2_19mer_win1_15886 | 15886 | 15904 | 474 | CATACAATGCTAGTTAAAC | 1677 | GTTTAACTAGCATTGTATG |
| NC_045512.2_19mer_win1_15887 | 15887 | 15905 | 475 | ATACAATGCTAGTTAAACA | 1678 | TGTTTAACTAGCATTGTAT |
| NC_045512.2_19mer_win1_15985 | 15985 | 16003 | 476 | AAAACAGATGGTACACTTA | 1679 | TAAGTGTACCATCTGTTTT |
| NC_045512.2_19mer_win1_15986 | 15986 | 16004 | 477 | AAACAGATGGTACACTTAT | 1680 | ATAAGTGTACCATCTGTTT |
| NC_045512.2_19mer_win1_15987 | 15987 | 16005 | 478 | AACAGATGGTACACTTATG | 1681 | CATAAGTGTACCATCTGTT |
| NC_045512.2_19mer_win1_15988 | 15988 | 16006 | 479 | ACAGATGGTACACTTATGA | 1682 | TCATAAGTGTACCATCTGT |
| NC_045512.2_19mer_win1_15989 | 15989 | 16007 | 480 | CAGATGGTACACTTATGAT | 1683 | ATCATAAGTGTACCATCTG |
| NC_045512.2_19mer_win1_15990 | 15990 | 16008 | 481 | AGATGGTACACTTATGATT | 1684 | AATCATAAGTGTACCATCT |
| NC_045512.2_19mer_win1_15991 | 15991 | 16009 | 482 | GATGGTACACTTATGATTG | 1685 | CAATCATAAGTGTACCATC |
| NC_045512.2_19mer_win1_15992 | 15992 | 16010 | 483 | ATGGTACACTTATGATTGA | 1686 | TCAATCATAAGTGTACCAT |
| NC_045512.2_19mer_win1_16057 | 16057 | 16075 | 484 | CCTAATCAGGAGTATGCTG | 1687 | CAGCATACTCCTGATTAGG |
| NC_045512.2_19mer_win1_16058 | 16058 | 16076 | 485 | CTAATCAGGAGTATGCTGA | 1688 | TCAGCATACTCCTGATTAG |
| NC_045512.2_19mer_win1_16059 | 16059 | 16077 | 486 | TAATCAGGAGTATGCTGAT | 1689 | ATCAGCATACTCCTGATTA |
| NC_045512.2_19mer_win1_16060 | 16060 | 16078 | 487 | AATCAGGAGTATGCTGATG | 1690 | CATCAGCATACTCCTGATT |
| NC_045512.2_19mer_win1_16061 | 16061 | 16079 | 488 | ATCAGGAGTATGCTGATGT | 1691 | ACATCAGCATACTCCTGAT |
| NC_045512.2_19mer_win1_16186 | 16186 | 16204 | 489 | TGGGAACCTGAGTTTTATG | 1692 | CATAAAACTCAGGTTCCCA |
| NC_045512.2_19mer_win1_16187 | 16187 | 16205 | 490 | GGGAACCTGAGTTTTATGA | 1693 | TCATAAAACTCAGGTTCCC |
| NC_045512.2_19mer_win1_16430 | 16430 | 16448 | 491 | TAGGAGGTATGAGCTATTA | 1694 | TAATAGCTCATACCTCCTA |
| NC_045512.2_19mer_win1_16822 | 16822 | 16840 | 492 | GGAGAGTACACCTTTGAAA | 1695 | TTTCAAAGGTGTACTCTCC |
| NC_045512.2_19mer_win1_16823 | 16823 | 16841 | 493 | GAGAGTACACCTTTGAAAA | 1696 | TTTTCAAAGGTGTACTCTC |
| NC_045512.2_19mer_win1_16824 | 16824 | 16842 | 494 | AGAGTACACCTTTGAAAAA | 1697 | TTTTTCAAAGGTGTACTCT |
| NC_045512.2_19mer_win1_16825 | 16825 | 16843 | 495 | GAGTACACCTTTGAAAAAG | 1698 | CTTTTTCAAAGGTGTACTC |
| NC_045512.2_19mer_win1_16826 | 16826 | 16844 | 496 | AGTACACCTTTGAAAAAGG | 1699 | CCTTTTTCAAAGGTGTACT |
| NC_045512.2_19mer_win1_16827 | 16827 | 16845 | 497 | GTACACCTTTGAAAAAGGT | 1700 | ACCTTTTTCAAAGGTGTAC |
| NC_045512.2_19mer_win1_16828 | 16828 | 16846 | 498 | TACACCTTTGAAAAAGGTG | 1701 | CACCTTTTTCAAAGGTGTA |
| NC_045512.2_19mer_win1_16829 | 16829 | 16847 | 499 | ACACCTTTGAAAAAGGTGA | 1702 | TCACCTTTTTCAAAGGTGT |
| NC_045512.2_19mer_win1_16830 | 16830 | 16848 | 500 | CACCTTTGAAAAAGGTGAC | 1703 | GTCACCTTTTTCAAAGGTG |
| NC_045512.2_19mer_win1_16831 | 16831 | 16849 | 501 | ACCTTTGAAAAAGGTGACT | 1704 | AGTCACCTTTTTCAAAGGT |
| NC_045512.2_19mer_win1_16832 | 16832 | 16850 | 502 | CCTTTGAAAAAGGTGACTA | 1705 | TAGTCACCTTTTTCAAAGG |
| NC_045512.2_19mer_win1_16833 | 16833 | 16851 | 503 | CTTTGAAAAAGGTGACTAT | 1706 | ATAGTCACCTTTTTCAAAG |
| NC_045512.2_19mer_win1_16834 | 16834 | 16852 | 504 | TTTGAAAAAGGTGACTATG | 1707 | CATAGTCACCTTTTTCAAA |
| NC_045512.2_19mer_win1_16835 | 16835 | 16853 | 505 | TTGAAAAAGGTGACTATGG | 1708 | CCATAGTCACCTTTTTCAA |
| NC_045512.2_19mer_win1_16836 | 16836 | 16854 | 506 | TGAAAAAGGTGACTATGGT | 1709 | ACCATAGTCACCTTTTTCA |
| NC_045512.2_19mer_win1_16837 | 16837 | 16855 | 507 | GAAAAAGGTGACTATGGTG | 1710 | CACCATAGTCACCTTTTTC |
| NC_045512.2_19mer_win1_16838 | 16838 | 16856 | 508 | AAAAAGGTGACTATGGTGA | 1711 | TCACCATAGTCACCTTTTT |
| NC_045512.2_19mer_win1_16839 | 16839 | 16857 | 509 | AAAAGGTGACTATGGTGAT | 1712 | ATCACCATAGTCACCTTTT |
| NC_045512.2_19mer_win1_16840 | 16840 | 16858 | 510 | AAAGGTGACTATGGTGATG | 1713 | CATCACCATAGTCACCTTT |
| NC_045512.2_19mer_win1_16841 | 16841 | 16859 | 511 | AAGGTGACTATGGTGATGC | 1714 | GCATCACCATAGTCACCTT |
| NC_045512.2_19mer_win1_16842 | 16842 | 16860 | 512 | AGGTGACTATGGTGATGCT | 1715 | AGCATCACCATAGTCACCT |
| NC_045512.2_19mer_win1_16843 | 16843 | 16861 | 513 | GGTGACTATGGTGATGCTG | 1716 | CAGCATCACCATAGTCACC |
| NC_045512.2_19mer_win1_16844 | 16844 | 16862 | 514 | GTGACTATGGTGATGCTGT | 1717 | ACAGCATCACCATAGTCAC |
| NC_045512.2_19mer_win1_16845 | 16845 | 16863 | 515 | TGACTATGGTGATGCTGTT | 1718 | AACAGCATCACCATAGTCA |
| NC_045512.2_19mer_win1_16846 | 16846 | 16864 | 516 | GACTATGGTGATGCTGTTG | 1719 | CAACAGCATCACCATAGTC |
| NC_045512.2_19mer_win1_16847 | 16847 | 16865 | 517 | ACTATGGTGATGCTGTTGT | 1720 | ACAACAGCATCACCATAGT |
| NC_045512.2_19mer_win1_16954 | 16954 | 16972 | 518 | CTAGTGCCACAAGAGCACT | 1721 | AGTGCTCTTGTGGCACTAG |
| NC_045512.2_19mer_win1_16955 | 16955 | 16973 | 519 | TAGTGCCACAAGAGCACTA | 1722 | TAGTGCTCTTGTGGCACTA |
| NC_045512.2_19mer_win1_16956 | 16956 | 16974 | 520 | AGTGCCACAAGAGCACTAT | 1723 | ATAGTGCTCTTGTGGCACT |
| NC_045512.2_19mer_win1_16957 | 16957 | 16975 | 521 | GTGCCACAAGAGCACTATG | 1724 | CATAGTGCTCTTGTGGCAC |
| NC_045512.2_19mer_win1_16958 | 16958 | 16976 | 522 | TGCCACAAGAGCACTATGT | 1725 | ACATAGTGCTCTTGTGGCA |
| NC_045512.2_19mer_win1_17008 | 17008 | 17026 | 523 | ATCTCAGATGAGTTTTCTA | 1726 | TAGAAAACTCATCTGAGAT |
| NC_045512.2_19mer_win1_17009 | 17009 | 17027 | 524 | TCTCAGATGAGTTTTCTAG | 1727 | CTAGAAAACTCATCTGAGA |
| NC_045512.2_19mer_win1_17010 | 17010 | 17028 | 525 | CTCAGATGAGTTTTCTAGC | 1728 | GCTAGAAAACTCATCTGAG |
| NC_045512.2_19mer_win1_17011 | 17011 | 17029 | 526 | TCAGATGAGTTTTCTAGCA | 1729 | TGCTAGAAAACTCATCTGA |
| NC_045512.2_19mer_win1_17012 | 17012 | 17030 | 527 | CAGATGAGTTTTCTAGCAA | 1730 | TTGCTAGAAAACTCATCTG |
| NC_045512.2_19mer_win1_17013 | 17013 | 17031 | 528 | AGATGAGTTTTCTAGCAAT | 1731 | ATTGCTAGAAAACTCATCT |
| NC_045512.2_19mer_win1_17014 | 17014 | 17032 | 529 | GATGAGTTTTCTAGCAATG | 1732 | CATTGCTAGAAAACTCATC |
| NC_045512.2_19mer_win1_17015 | 17015 | 17033 | 530 | ATGAGTTTTCTAGCAATGT | 1733 | ACATTGCTAGAAAACTCAT |
| NC_045512.2_19mer_win1_17016 | 17016 | 17034 | 531 | TGAGTTTTCTAGCAATGTT | 1734 | AACATTGCTAGAAAACTCA |
| NC_045512.2_19mer_win1_17017 | 17017 | 17035 | 532 | GAGTTTTCTAGCAATGTTG | 1735 | CAACATTGCTAGAAAACTC |
| NC_045512.2_19mer_win1_17018 | 17018 | 17036 | 533 | AGTTTTCTAGCAATGTTGC | 1736 | GCAACATTGCTAGAAAACT |
| NC_045512.2_19mer_win1_17019 | 17019 | 17037 | 534 | GTTTTCTAGCAATGTTGCA | 1737 | TGCAACATTGCTAGAAAAC |
| NC_045512.2_19mer_win1_17020 | 17020 | 17038 | 535 | TTTTCTAGCAATGTTGCAA | 1738 | TTGCAACATTGCTAGAAAA |
| NC_045512.2_19mer_win1_17021 | 17021 | 17039 | 536 | TTTCTAGCAATGTTGCAAA | 1739 | TTTGCAACATTGCTAGAAA |
| NC_045512.2_19mer_win1_17022 | 17022 | 17040 | 537 | TTCTAGCAATGTTGCAAAT | 1740 | ATTTGCAACATTGCTAGAA |
| NC_045512.2_19mer_win1_17023 | 17023 | 17041 | 538 | TCTAGCAATGTTGCAAATT | 1741 | AATTTGCAACATTGCTAGA |
| NC_045512.2_19mer_win1_17024 | 17024 | 17042 | 539 | CTAGCAATGTTGCAAATTA | 1742 | TAATTTGCAACATTGCTAG |
| NC_045512.2_19mer_win1_17080 | 17080 | 17098 | 540 | GGACCACCTGGTACTGGTA | 1743 | TACCAGTACCAGGTGGTCC |
| NC_045512.2_19mer_win1_17081 | 17081 | 17099 | 541 | GACCACCTGGTACTGGTAA | 1744 | TTACCAGTACCAGGTGGTC |
| NC_045512.2_19mer_win1_17082 | 17082 | 17100 | 542 | ACCACCTGGTACTGGTAAG | 1745 | CTTACCAGTACCAGGTGGT |
| NC_045512.2_19mer_win1_17083 | 17083 | 17101 | 543 | CCACCTGGTACTGGTAAGA | 1746 | TCTTACCAGTACCAGGTGG |
| NC_045512.2_19mer_win1_17084 | 17084 | 17102 | 544 | CACCTGGTACTGGTAAGAG | 1747 | CTCTTACCAGTACCAGGTG |
| NC_045512.2_19mer_win1_17085 | 17085 | 17103 | 545 | ACCTGGTACTGGTAAGAGT | 1748 | ACTCTTACCAGTACCAGGT |
| NC_045512.2_19mer_win1_17086 | 17086 | 17104 | 546 | CCTGGTACTGGTAAGAGTC | 1749 | GACTCTTACCAGTACCAGG |
| NC_045512.2_19mer_win1_17087 | 17087 | 17105 | 547 | CTGGTACTGGTAAGAGTCA | 1750 | TGACTCTTACCAGTACCAG |
| NC_045512.2_19mer_win1_17088 | 17088 | 17106 | 548 | TGGTACTGGTAAGAGTCAT | 1751 | ATGACTCTTACCAGTACCA |
| NC_045512.2_19mer_win1_17089 | 17089 | 17107 | 549 | GGTACTGGTAAGAGTCATT | 1752 | AATGACTCTTACCAGTACC |
| NC_045512.2_19mer_win1_17090 | 17090 | 17108 | 550 | GTACTGGTAAGAGTCATTT | 1753 | AAATGACTCTTACCAGTAC |
| NC_045512.2_19mer_win1_17091 | 17091 | 17109 | 551 | TACTGGTAAGAGTCATTTT | 1754 | AAAATGACTCTTACCAGTA |
| NC_045512.2_19mer_win1_17092 | 17092 | 17110 | 552 | ACTGGTAAGAGTCATTTTG | 1755 | CAAAATGACTCTTACCAGT |
| NC_045512.2_19mer_win1_17093 | 17093 | 17111 | 553 | CTGGTAAGAGTCATTTTGC | 1756 | GCAAAATGACTCTTACCAG |
| NC_045512.2_19mer_win1_17137 | 17137 | 17155 | 554 | TCTGCTCGCATAGTGTATA | 1757 | TATACACTATGCGAGCAGA |
| NC_045512.2_19mer_win1_17138 | 17138 | 17156 | 555 | CTGCTCGCATAGTGTATAC | 1758 | GTATACACTATGCGAGCAG |
| NC_045512.2_19mer_win1_17269 | 17269 | 17287 | 556 | AAATTCAAAGTGAATTCAA | 1759 | TTGAATTCACTTTGAATTT |
| NC_045512.2_19mer_win1_17270 | 17270 | 17288 | 557 | AATTCAAAGTGAATTCAAC | 1760 | GTTGAATTCACTTTGAATT |
| NC_045512.2_19mer_win1_17271 | 17271 | 17289 | 558 | ATTCAAAGTGAATTCAACA | 1761 | TGTTGAATTCACTTTGAAT |
| NC_045512.2_19mer_win1_17530 | 17530 | 17548 | 559 | ATAGGTCCAGACATGTTCC | 1762 | GGAACATGTCTGGACCTAT |
| NC_045512.2_19mer_win1_17531 | 17531 | 17549 | 560 | TAGGTCCAGACATGTTCCT | 1763 | AGGAACATGTCTGGACCTA |
| NC_045512.2_19mer_win1_17563 | 17563 | 17581 | 561 | CGTTGTCCTGCTGAAATTG | 1764 | CAATTTCAGCAGGACAACG |
| NC_045512.2_19mer_win1_17564 | 17564 | 17582 | 562 | GTTGTCCTGCTGAAATTGT | 1765 | ACAATTTCAGCAGGACAAC |
| NC_045512.2_19mer_win1_17680 | 17680 | 17698 | 563 | CATGATGTTTCATCTGCAA | 1766 | TTGCAGATGAAACATCATG |
| NC_045512.2_19mer_win1_17681 | 17681 | 17699 | 564 | ATGATGTTTCATCTGCAAT | 1767 | ATTGCAGATGAAACATCAT |
| NC_045512.2_19mer_win1_17746 | 17746 | 17764 | 565 | CCTGCTTGGAGAAAAGCTG | 1768 | CAGCTTTTCTCCAAGCAGG |
| NC_045512.2_19mer_win1_17747 | 17747 | 17765 | 566 | CTGCTTGGAGAAAAGCTGT | 1769 | ACAGCTTTTCTCCAAGCAG |
| NC_045512.2_19mer_win1_17857 | 17857 | 17875 | 567 | TATGACTATGTCATATTCA | 1770 | TGAATATGACATAGTCATA |
| NC_045512.2_19mer_win1_17858 | 17858 | 17876 | 568 | ATGACTATGTCATATTCAC | 1771 | GTGAATATGACATAGTCAT |
| NC_045512.2_19mer_win1_17956 | 17956 | 17974 | 569 | TGCATAATGTCTGATAGAG | 1772 | CTCTATCAGACATTATGCA |
| NC_045512.2_19mer_win1_17957 | 17957 | 17975 | 570 | GCATAATGTCTGATAGAGA | 1773 | TCTCTATCAGACATTATGC |
| NC_045512.2_19mer_win1_18100 | 18100 | 18118 | 571 | ACACAGGCACCTACACACC | 1774 | GGTGTGTAGGTGCCTGTGT |
| NC_045512.2_19mer_win1_18101 | 18101 | 18119 | 572 | CACAGGCACCTACACACCT | 1775 | AGGTGTGTAGGTGCCTGTG |
| NC_045512.2_19mer_win1_18102 | 18102 | 18120 | 573 | ACAGGCACCTACACACCTC | 1776 | GAGGTGTGTAGGTGCCTGT |
| NC_045512.2_19mer_win1_18103 | 18103 | 18121 | 574 | CAGGCACCTACACACCTCA | 1777 | TGAGGTGTGTAGGTGCCTG |
| NC_045512.2_19mer_win1_18104 | 18104 | 18122 | 575 | AGGCACCTACACACCTCAG | 1778 | CTGAGGTGTGTAGGTGCCT |
| NC_045512.2_19mer_win1_18196 | 18196 | 18214 | 576 | AGACTCATCTCTATGATGG | 1779 | CCATCATAGAGATGAGTCT |
| NC_045512.2_19mer_win1_18197 | 18197 | 18215 | 577 | GACTCATCTCTATGATGGG | 1780 | CCCATCATAGAGATGAGTC |
| NC_045512.2_19mer_win1_18198 | 18198 | 18216 | 578 | ACTCATCTCTATGATGGGT | 1781 | ACCCATCATAGAGATGAGT |
| NC_045512.2_19mer_win1_18199 | 18199 | 18217 | 579 | CTCATCTCTATGATGGGTT | 1782 | AACCCATCATAGAGATGAG |
| NC_045512.2_19mer_win1_18200 | 18200 | 18218 | 580 | TCATCTCTATGATGGGTTT | 1783 | AAACCCATCATAGAGATGA |
| NC_045512.2_19mer_win1_19618 | 19618 | 19636 | 581 | CAGAGTTTAGAAAATGTGG | 1784 | CCACATTTTCTAAACTCTG |
| NC_045512.2_19mer_win1_19619 | 19619 | 19637 | 582 | AGAGTTTAGAAAATGTGGC | 1785 | GCCACATTTTCTAAACTCT |
| NC_045512.2_19mer_win1_19620 | 19620 | 19638 | 583 | GAGTTTAGAAAATGTGGCT | 1786 | AGCCACATTTTCTAAACTC |
| NC_045512.2_19mer_win1_19621 | 19621 | 19639 | 584 | AGTTTAGAAAATGTGGCTT | 1787 | AAGCCACATTTTCTAAACT |
| NC_045512.2_19mer_win1_19783 | 19783 | 19801 | 585 | TTTGAGCTTTGGGCTAAGC | 1788 | GCTTAGCCCAAAGCTCAAA |
| NC_045512.2_19mer_win1_19784 | 19784 | 19802 | 586 | TTGAGCTTTGGGCTAAGCG | 1789 | CGCTTAGCCCAAAGCTCAA |
| NC_045512.2_19mer_win1_19831 | 19831 | 19849 | 587 | ATACTCAATAATTTGGGTG | 1790 | CACCCAAATTATTGAGTAT |
| NC_045512.2_19mer_win1_19832 | 19832 | 19850 | 588 | TACTCAATAATTTGGGTGT | 1791 | ACACCCAAATTATTGAGTA |
| NC_045512.2_19mer_win1_20107 | 20107 | 20125 | 589 | AATGGAGTCACATTAATTG | 1792 | CAATTAATGTGACTCCATT |
| NC_045512.2_19mer_win1_20108 | 20108 | 20126 | 590 | ATGGAGTCACATTAATTGG | 1793 | CCAATTAATGTGACTCCAT |
| NC_045512.2_19mer_win1_20109 | 20109 | 20127 | 591 | TGGAGTCACATTAATTGGA | 1794 | TCCAATTAATGTGACTCCA |
| NC_045512.2_19mer_win1_20110 | 20110 | 20128 | 592 | GGAGTCACATTAATTGGAG | 1795 | CTCCAATTAATGTGACTCC |
| NC_045512.2_19mer_win1_20111 | 20111 | 20129 | 593 | GAGTCACATTAATTGGAGA | 1796 | TCTCCAATTAATGTGACTC |
| NC_045512.2_19mer_win1_20112 | 20112 | 20130 | 594 | AGTCACATTAATTGGAGAA | 1797 | TTCTCCAATTAATGTGACT |
| NC_045512.2_19mer_win1_20776 | 20776 | 20794 | 595 | ATAATGATGAATGTCGCAA | 1798 | TTGCGACATTCATCATTAT |
| NC_045512.2_19mer_win1_20777 | 20777 | 20795 | 596 | TAATGATGAATGTCGCAAA | 1799 | TTTGCGACATTCATCATTA |
| NC_045512.2_19mer_win1_21502 | 21502 | 21520 | 597 | ATTAGAGAAAACAACAGAG | 1800 | CTCTGTTGTTTTCTCTAAT |
| NC_045512.2_19mer_win1_21503 | 21503 | 21521 | 598 | TTAGAGAAAACAACAGAGT | 1801 | ACTCTGTTGTTTTCTCTAA |
| NC_045512.2_19mer_win1_21504 | 21504 | 21522 | 599 | TAGAGAAAACAACAGAGTT | 1802 | AACTCTGTTGTTTTCTCTA |
| NC_045512.2_19mer_win1_21505 | 21505 | 21523 | 600 | AGAGAAAACAACAGAGTTG | 1803 | CAACTCTGTTGTTTTCTCT |
| NC_045512.2_19mer_win1_21506 | 21506 | 21524 | 601 | GAGAAAACAACAGAGTTGT | 1804 | ACAACTCTGTTGTTTTCTC |
| NC_045512.2_19mer_win1_24302 | 24302 | 24320 | 602 | AATGTTCTCTATGAGAACC | 1805 | GGTTCTCATAGAGAACATT |
| NC_045512.2_19mer_win1_24303 | 24303 | 24321 | 603 | ATGTTCTCTATGAGAACCA | 1806 | TGGTTCTCATAGAGAACAT |
| NC_045512.2_19mer_win1_24304 | 24304 | 24322 | 604 | TGTTCTCTATGAGAACCAA | 1807 | TTGGTTCTCATAGAGAACA |
| NC_045512.2_19mer_win1_24305 | 24305 | 24323 | 605 | GTTCTCTATGAGAACCAAA | 1808 | TTTGGTTCTCATAGAGAAC |
| NC_045512.2_19mer_win1_24306 | 24306 | 24324 | 606 | TTCTCTATGAGAACCAAAA | 1809 | TTTTGGTTCTCATAGAGAA |
| NC_045512.2_19mer_win1_24307 | 24307 | 24325 | 607 | TCTCTATGAGAACCAAAAA | 1810 | TTTTTGGTTCTCATAGAGA |
| NC_045512.2_19mer_win1_24446 | 24446 | 24464 | 608 | CTTGTTAAACAACTTAGCT | 1811 | AGCTAAGTTGTTTAACAAG |
| NC_045512.2_19mer_win1_24447 | 24447 | 24465 | 609 | TTGTTAAACAACTTAGCTC | 1812 | GAGCTAAGTTGTTTAACAA |
| NC_045512.2_19mer_win1_24620 | 24620 | 24638 | 610 | GCTTCTGCTAATCTTGCTG | 1813 | CAGCAAGATTAGCAGAAGC |
| NC_045512.2_19mer_win1_24621 | 24621 | 24639 | 611 | CTTCTGCTAATCTTGCTGC | 1814 | GCAGCAAGATTAGCAGAAG |
| NC_045512.2_19mer_win1_24622 | 24622 | 24640 | 612 | TTCTGCTAATCTTGCTGCT | 1815 | AGCAGCAAGATTAGCAGAA |
| NC_045512.2_19mer_win1_24623 | 24623 | 24641 | 613 | TCTGCTAATCTTGCTGCTA | 1816 | TAGCAGCAAGATTAGCAGA |
| NC_045512.2_19mer_win1_24624 | 24624 | 24642 | 614 | CTGCTAATCTTGCTGCTAC | 1817 | GTAGCAGCAAGATTAGCAG |
| NC_045512.2_19mer_win1_24625 | 24625 | 24643 | 615 | TGCTAATCTTGCTGCTACT | 1818 | AGTAGCAGCAAGATTAGCA |
| NC_045512.2_19mer_win1_24626 | 24626 | 24644 | 616 | GCTAATCTTGCTGCTACTA | 1819 | TAGTAGCAGCAAGATTAGC |
| NC_045512.2_19mer_win1_24627 | 24627 | 24645 | 617 | CTAATCTTGCTGCTACTAA | 1820 | TTAGTAGCAGCAAGATTAG |
| NC_045512.2_19mer_win1_24628 | 24628 | 24646 | 618 | TAATCTTGCTGCTACTAAA | 1821 | TTTAGTAGCAGCAAGATTA |
| NC_045512.2_19mer_win1_24629 | 24629 | 24647 | 619 | AATCTTGCTGCTACTAAAA | 1822 | TTTTAGTAGCAGCAAGATT |
| NC_045512.2_19mer_win1_24630 | 24630 | 24648 | 620 | ATCTTGCTGCTACTAAAAT | 1823 | ATTTTAGTAGCAGCAAGAT |
| NC_045512.2_19mer_win1_24631 | 24631 | 24649 | 621 | TCTTGCTGCTACTAAAATG | 1824 | CATTTTAGTAGCAGCAAGA |
| NC_045512.2_19mer_win1_24632 | 24632 | 24650 | 622 | CTTGCTGCTACTAAAATGT | 1825 | ACATTTTAGTAGCAGCAAG |
| NC_045512.2_19mer_win1_24633 | 24633 | 24651 | 623 | TTGCTGCTACTAAAATGTC | 1826 | GACATTTTAGTAGCAGCAA |
| NC_045512.2_19mer_win1_24662 | 24662 | 24680 | 624 | CTTGGACAATCAAAAAGAG | 1827 | CTCTTTTTGATTGTCCAAG |
| NC_045512.2_19mer_win1_24663 | 24663 | 24681 | 625 | TTGGACAATCAAAAAGAGT | 1828 | ACTCTTTTTGATTGTCCAA |
| NC_045512.2_19mer_win1_24664 | 24664 | 24682 | 626 | TGGACAATCAAAAAGAGTT | 1829 | AACTCTTTTTGATTGTCCA |
| NC_045512.2_19mer_win1_24665 | 24665 | 24683 | 627 | GGACAATCAAAAAGAGTTG | 1830 | CAACTCTTTTTGATTGTCC |
| NC_045512.2_19mer_win1_24666 | 24666 | 24684 | 628 | GACAATCAAAAAGAGTTGA | 1831 | TCAACTCTTTTTGATTGTC |
| NC_045512.2_19mer_win1_25034 | 25034 | 25052 | 629 | AATCATACATCACCAGATG | 1832 | CATCTGGTGATGTATGATT |
| NC_045512.2_19mer_win1_25035 | 25035 | 25053 | 630 | ATCATACATCACCAGATGT | 1833 | ACATCTGGTGATGTATGAT |
| NC_045512.2_19mer_win1_25038 | 25038 | 25056 | 633 | ATACATCACCAGATGTTGA | 1836 | TCAACATCTGGTGATGTAT |
| NC_045512.2_19mer_win1_25039 | 25039 | 25057 | 634 | TACATCACCAGATGTTGAT | 1837 | ATCAACATCTGGTGATGTA |
| NC_045512.2_19mer_win1_25104 | 25104 | 25122 | 635 | AAGAAATTGACCGCCTCAA | 1838 | TTGAGGCGGTCAATTTCTT |
| NC_045512.2_19mer_win1_25105 | 25105 | 25123 | 636 | AGAAATTGACCGCCTCAAT | 1839 | ATTGAGGCGGTCAATTTCT |
| NC_045512.2_19mer_win1_25106 | 25106 | 25124 | 637 | GAAATTGACCGCCTCAATG | 1840 | CATTGAGGCGGTCAATTTC |
| NC_045512.2_19mer_win1_25107 | 25107 | 25125 | 638 | AAATTGACCGCCTCAATGA | 1841 | TCATTGAGGCGGTCAATTT |
| NC_045512.2_19mer_win1_25108 | 25108 | 25126 | 639 | AATTGACCGCCTCAATGAG | 1842 | CTCATTGAGGCGGTCAATT |
| NC_045512.2_19mer_win1_25109 | 25109 | 25127 | 640 | ATTGACCGCCTCAATGAGG | 1843 | CCTCATTGAGGCGGTCAAT |
| NC_045512.2_19mer_win1_25110 | 25110 | 25128 | 641 | TTGACCGCCTCAATGAGGT | 1844 | ACCTCATTGAGGCGGTCAA |
| NC_045512.2_19mer_win1_25364 | 25364 | 25382 | 642 | GTCAAATTACATTACACAT | 1845 | ATGTGTAATGTAATTTGAC |
| NC_045512.2_19mer_win1_25365 | 25365 | 25383 | 643 | TCAAATTACATTACACATA | 1846 | TATGTGTAATGTAATTTGA |
| NC_045512.2_19mer_win1_25366 | 25366 | 25384 | 644 | CAAATTACATTACACATAA | 1847 | TTATGTGTAATGTAATTTG |
| NC_045512.2_19mer_win1_25367 | 25367 | 25385 | 645 | AAATTACATTACACATAAA | 1848 | TTTATGTGTAATGTAATTT |
| NC_045512.2_19mer_win1_25368 | 25368 | 25386 | 646 | AATTACATTACACATAAAC | 1849 | GTTTATGTGTAATGTAATT |
| NC_045512.2_19mer_win1_25369 | 25369 | 25387 | 647 | ATTACATTACACATAAACG | 1850 | CGTTTATGTGTAATGTAAT |
| NC_045512.2_19mer_win1_25502 | 25502 | 25520 | 648 | TACAAGCCTCACTCCCTTT | 1851 | AAAGGGAGTGAGGCTTGTA |
| NC_045512.2_19mer_win1_25503 | 25503 | 25521 | 649 | ACAAGCCTCACTCCCTTTC | 1852 | GAAAGGGAGTGAGGCTTGT |
| NC_045512.2_19mer_win1_25504 | 25504 | 25522 | 650 | CAAGCCTCACTCCCTTTCG | 1853 | CGAAAGGGAGTGAGGCTTG |
| NC_045512.2_19mer_win1_25505 | 25505 | 25523 | 651 | AAGCCTCACTCCCTTTCGG | 1854 | CCGAAAGGGAGTGAGGCTT |
| NC_045512.2_19mer_win1_25506 | 25506 | 25524 | 652 | AGCCTCACTCCCTTTCGGA | 1855 | TCCGAAAGGGAGTGAGGCT |
| NC_045512.2_19mer_win1_25507 | 25507 | 25525 | 653 | GCCTCACTCCCTTTCGGAT | 1856 | ATCCGAAAGGGAGTGAGGC |
| NC_045512.2_19mer_win1_25508 | 25508 | 25526 | 654 | CCTCACTCCCTTTCGGATG | 1857 | CATCCGAAAGGGAGTGAGG |
| NC_045512.2_19mer_win1_25509 | 25509 | 25527 | 655 | CTCACTCCCTTTCGGATGG | 1858 | CCATCCGAAAGGGAGTGAG |
| NC_045512.2_19mer_win1_25510 | 25510 | 25528 | 656 | TCACTCCCTTTCGGATGGC | 1859 | GCCATCCGAAAGGGAGTGA |
| NC_045512.2_19mer_win1_25511 | 25511 | 25529 | 657 | CACTCCCTTTCGGATGGCT | 1860 | AGCCATCCGAAAGGGAGTG |
| NC_045512.2_19mer_win1_25512 | 25512 | 25530 | 658 | ACTCCCTTTCGGATGGCTT | 1861 | AAGCCATCCGAAAGGGAGT |
| NC_045512.2_19mer_win1_26191 | 26191 | 26209 | 659 | CCGACGACGACTACTAGCG | 1862 | CGCTAGTAGTCGTCGTCGG |
| NC_045512.2_19mer_win1_26192 | 26192 | 26210 | 660 | CGACGACGACTACTAGCGT | 1863 | ACGCTAGTAGTCGTCGTCG |
| NC_045512.2_19mer_win1_26193 | 26193 | 26211 | 661 | GACGACGACTACTAGCGTG | 1864 | CACGCTAGTAGTCGTCGTC |
| NC_045512.2_19mer_win1_26194 | 26194 | 26212 | 662 | ACGACGACTACTAGCGTGC | 1865 | GCACGCTAGTAGTCGTCGT |
| NC_045512.2_19mer_win1_26195 | 26195 | 26213 | 663 | CGACGACTACTAGCGTGCC | 1866 | GGCACGCTAGTAGTCGTCG |
| NC_045512.2_19mer_win1_26196 | 26196 | 26214 | 664 | GACGACTACTAGCGTGCCT | 1867 | AGGCACGCTAGTAGTCGTC |
| NC_045512.2_19mer_win1_26197 | 26197 | 26215 | 665 | ACGACTACTAGCGTGCCTT | 1868 | AAGGCACGCTAGTAGTCGT |
| NC_045512.2_19mer_win1_26198 | 26198 | 26216 | 666 | CGACTACTAGCGTGCCTTT | 1869 | AAAGGCACGCTAGTAGTCG |
| NC_045512.2_19mer_win1_26199 | 26199 | 26217 | 667 | GACTACTAGCGTGCCTTTG | 1870 | CAAAGGCACGCTAGTAGTC |
| NC_045512.2_19mer_win1_26200 | 26200 | 26218 | 668 | ACTACTAGCGTGCCTTTGT | 1871 | ACAAAGGCACGCTAGTAGT |
| NC_045512.2_19mer_win1_26201 | 26201 | 26219 | 669 | CTACTAGCGTGCCTTTGTA | 1872 | TACAAAGGCACGCTAGTAG |
| NC_045512.2_19mer_win1_26202 | 26202 | 26220 | 670 | TACTAGCGTGCCTTTGTAA | 1873 | TTACAAAGGCACGCTAGTA |
| NC_045512.2_19mer_win1_26203 | 26203 | 26221 | 671 | ACTAGCGTGCCTTTGTAAG | 1874 | CTTACAAAGGCACGCTAGT |
| NC_045512.2_19mer_win1_26204 | 26204 | 26222 | 672 | CTAGCGTGCCTTTGTAAGC | 1875 | GCTTACAAAGGCACGCTAG |
| NC_045512.2_19mer_win1_26205 | 26205 | 26223 | 673 | TAGCGTGCCTTTGTAAGCA | 1876 | TGCTTACAAAGGCACGCTA |
| NC_045512.2_19mer_win1_26206 | 26206 | 26224 | 674 | AGCGTGCCTTTGTAAGCAC | 1877 | GTGCTTACAAAGGCACGCT |
| NC_045512.2_19mer_win1_26207 | 26207 | 26225 | 675 | GCGTGCCTTTGTAAGCACA | 1878 | TGTGCTTACAAAGGCACGC |
| NC_045512.2_19mer_win1_26208 | 26208 | 26226 | 676 | CGTGCCTTTGTAAGCACAA | 1879 | TTGTGCTTACAAAGGCACG |
| NC_045512.2_19mer_win1_26209 | 26209 | 26227 | 677 | GTGCCTTTGTAAGCACAAG | 1880 | CTTGTGCTTACAAAGGCAC |
| NC_045512.2_19mer_win1_26232 | 26232 | 26250 | 678 | TGAGTACGAACTTATGTAC | 1881 | GTACATAAGTTCGTACTCA |
| NC_045512.2_19mer_win1_26233 | 26233 | 26251 | 679 | GAGTACGAACTTATGTACT | 1882 | AGTACATAAGTTCGTACTC |
| NC_045512.2_19mer_win1_26234 | 26234 | 26252 | 680 | AGTACGAACTTATGTACTC | 1883 | GAGTACATAAGTTCGTACT |
| NC_045512.2_19mer_win1_26235 | 26235 | 26253 | 681 | GTACGAACTTATGTACTCA | 1884 | TGAGTACATAAGTTCGTAC |
| NC_045512.2_19mer_win1_26236 | 26236 | 26254 | 682 | TACGAACTTATGTACTCAT | 1885 | ATGAGTACATAAGTTCGTA |
| NC_045512.2_19mer_win1_26237 | 26237 | 26255 | 683 | ACGAACTTATGTACTCATT | 1886 | AATGAGTACATAAGTTCGT |
| NC_045512.2_19mer_win1_26238 | 26238 | 26256 | 684 | CGAACTTATGTACTCATTC | 1887 | GAATGAGTACATAAGTTCG |
| NC_045512.2_19mer_win1_26239 | 26239 | 26257 | 685 | GAACTTATGTACTCATTCG | 1888 | CGAATGAGTACATAAGTTC |
| NC_045512.2_19mer_win1_26240 | 26240 | 26258 | 686 | AACTTATGTACTCATTCGT | 1889 | ACGAATGAGTACATAAGTT |
| NC_045512.2_19mer_win1_26241 | 26241 | 26259 | 687 | ACTTATGTACTCATTCGTT | 1890 | AACGAATGAGTACATAAGT |
| NC_045512.2_19mer_win1_26242 | 26242 | 26260 | 688 | CTTATGTACTCATTCGTTT | 1891 | AAACGAATGAGTACATAAG |
| NC_045512.2_19mer_win1_26243 | 26243 | 26261 | 689 | TTATGTACTCATTCGTTTC | 1892 | GAAACGAATGAGTACATAA |
| NC_045512.2_19mer_win1_26244 | 26244 | 26262 | 690 | TATGTACTCATTCGTTTCG | 1893 | CGAAACGAATGAGTACATA |
| NC_045512.2_19mer_win1_26245 | 26245 | 26263 | 691 | ATGTACTCATTCGTTTCGG | 1894 | CCGAAACGAATGAGTACAT |
| NC_045512.2_19mer_win1_26246 | 26246 | 26264 | 692 | TGTACTCATTCGTTTCGGA | 1895 | TCCGAAACGAATGAGTACA |
| NC_045512.2_19mer_win1_26247 | 26247 | 26265 | 693 | GTACTCATTCGTTTCGGAA | 1896 | TTCCGAAACGAATGAGTAC |
| NC_045512.2_19mer_win1_26248 | 26248 | 26266 | 694 | TACTCATTCGTTTCGGAAG | 1897 | CTTCCGAAACGAATGAGTA |
| NC_045512.2_19mer_win1_26249 | 26249 | 26267 | 695 | ACTCATTCGTTTCGGAAGA | 1898 | TCTTCCGAAACGAATGAGT |
| NC_045512.2_19mer_win1_26269 | 26269 | 26287 | 696 | ACAGGTACGTTAATAGTTA | 1899 | TAACTATTAACGTACCTGT |
| NC_045512.2_19mer_win1_26270 | 26270 | 26288 | 697 | CAGGTACGTTAATAGTTAA | 1900 | TTAACTATTAACGTACCTG |
| NC_045512.2_19mer_win1_26271 | 26271 | 26289 | 698 | AGGTACGTTAATAGTTAAT | 1901 | ATTAACTATTAACGTACCT |
| NC_045512.2_19mer_win1_26272 | 26272 | 26290 | 699 | GGTACGTTAATAGTTAATA | 1902 | TATTAACTATTAACGTACC |
| NC_045512.2_19mer_win1_26273 | 26273 | 26291 | 700 | GTACGTTAATAGTTAATAG | 1903 | CTATTAACTATTAACGTAC |
| NC_045512.2_19mer_win1_26274 | 26274 | 26292 | 701 | TACGTTAATAGTTAATAGC | 1904 | GCTATTAACTATTAACGTA |
| NC_045512.2_19mer_win1_26275 | 26275 | 26293 | 702 | ACGTTAATAGTTAATAGCG | 1905 | CGCTATTAACTATTAACGT |
| NC_045512.2_19mer_win1_26276 | 26276 | 26294 | 703 | CGTTAATAGTTAATAGCGT | 1906 | ACGCTATTAACTATTAACG |
| NC_045512.2_19mer_win1_26277 | 26277 | 26295 | 704 | GTTAATAGTTAATAGCGTA | 1907 | TACGCTATTAACTATTAAC |
| NC_045512.2_19mer_win1_26278 | 26278 | 26296 | 705 | TTAATAGTTAATAGCGTAC | 1908 | GTACGCTATTAACTATTAA |
| NC_045512.2_19mer_win1_26279 | 26279 | 26297 | 706 | TAATAGTTAATAGCGTACT | 1909 | AGTACGCTATTAACTATTA |
| NC_045512.2_19mer_win1_26280 | 26280 | 26298 | 707 | AATAGTTAATAGCGTACTT | 1910 | AAGTACGCTATTAACTATT |
| NC_045512.2_19mer_win1_26281 | 26281 | 26299 | 708 | ATAGTTAATAGCGTACTTC | 1911 | GAAGTACGCTATTAACTAT |
| NC_045512.2_19mer_win1_26282 | 26282 | 26300 | 709 | TAGTTAATAGCGTACTTCT | 1912 | AGAAGTACGCTATTAACTA |
| NC_045512.2_19mer_win1_26283 | 26283 | 26301 | 710 | AGTTAATAGCGTACTTCTT | 1913 | AAGAAGTACGCTATTAACT |
| NC_045512.2_19mer_win1_26284 | 26284 | 26302 | 711 | GTTAATAGCGTACTTCTTT | 1914 | AAAGAAGTACGCTATTAAC |
| NC_045512.2_19mer_win1_26285 | 26285 | 26303 | 712 | TTAATAGCGTACTTCTTTT | 1915 | AAAAGAAGTACGCTATTAA |
| NC_045512.2_19mer_win1_26286 | 26286 | 26304 | 713 | TAATAGCGTACTTCTTTTT | 1916 | AAAAAGAAGTACGCTATTA |
| NC_045512.2_19mer_win1_26287 | 26287 | 26305 | 714 | AATAGCGTACTTCTTTTTC | 1917 | GAAAAAGAAGTACGCTATT |
| NC_045512.2_19mer_win1_26288 | 26288 | 26306 | 715 | ATAGCGTACTTCTTTTTCT | 1918 | AGAAAAAGAAGTACGCTAT |
| NC_045512.2_19mer_win1_26289 | 26289 | 26307 | 716 | TAGCGTACTTCTTTTTCTT | 1919 | AAGAAAAAGAAGTACGCTA |
| NC_045512.2_19mer_win1_26290 | 26290 | 26308 | 717 | AGCGTACTTCTTTTTCTTG | 1920 | CAAGAAAAAGAAGTACGCT |
| NC_045512.2_19mer_win1_26291 | 26291 | 26309 | 718 | GCGTACTTCTTTTTCTTGC | 1921 | GCAAGAAAAAGAAGTACGC |
| NC_045512.2_19mer_winl_26292 | 26292 | 26310 | 719 | CGTACTTCTTTTTCTTGCT | 1922 | AGCAAGAAAAAGAAGTACG |
| NC_045512.2_19mer_winl_26293 | 26293 | 26311 | 720 | GTACTTCTTTTTCTTGCTT | 1923 | AAGCAAGAAAAAGAAGTAC |
| NC_045512.2_19mer_winl_26294 | 26294 | 26312 | 721 | TACTTCTTTTTCTTGCTTT | 1924 | AAAGCAAGAAAAAGAAGTA |
| NC_045512.2_19mer_winl_26295 | 26295 | 26313 | 722 | ACTTCTTTTTCTTGCTTTC | 1925 | GAAAGCAAGAAAAAGAAGT |
| NC_045512.2_19mer_winl_26296 | 26296 | 26314 | 723 | CTTCTTTTTCTTGCTTTCG | 1926 | CGAAAGCAAGAAAAAGAAG |
| NC_045512.2_19mer_winl_26297 | 26297 | 26315 | 724 | TTCTTTTTCTTGCTTTCGT | 1927 | ACGAAAGCAAGAAAAAGAA |
| NC_045512.2_19mer_winl_26298 | 26298 | 26316 | 725 | TCTTTTTCTTGCTTTCGTG | 1928 | CACGAAAGCAAGAAAAAGA |
| NC_045512.2_19mer_winl_26299 | 26299 | 26317 | 726 | CTTTTTCTTGCTTTCGTGG | 1929 | CCACGAAAGCAAGAAAAAG |
| NC_045512.2_19mer_winl_26300 | 26300 | 26318 | 727 | TTTTTCTTGCTTTCGTGGT | 1930 | ACCACGAAAGCAAGAAAAA |
| NC_045512.2_19mer_winl_26301 | 26301 | 26319 | 728 | TTTTCTTGCTTTCGTGGTA | 1931 | TACCACGAAAGCAAGAAAA |
| NC_045512.2_19mer_winl_26302 | 26302 | 26320 | 729 | TTTCTTGCTTTCGTGGTAT | 1932 | ATACCACGAAAGCAAGAAA |
| NC_045512.2_19mer_winl_26303 | 26303 | 26321 | 730 | TTCTTGCTTTCGTGGTATT | 1933 | AATACCACGAAAGCAAGAA |
| NC_045512.2_19mer_winl_26304 | 26304 | 26322 | 731 | TCTTGCTTTCGTGGTATTC | 1934 | GAATACCACGAAAGCAAGA |
| NC_045512.2_19mer_winl_26305 | 26305 | 26323 | 732 | CTTGCTTTCGTGGTATTCT | 1935 | AGAATACCACGAAAGCAAG |
| NC_045512.2_19mer_winl_26306 | 26306 | 26324 | 733 | TTGCTTTCGTGGTATTCTT | 1936 | AAGAATACCACGAAAGCAA |
| NC_045512.2_19mer_winl_26307 | 26307 | 26325 | 734 | TGCTTTCGTGGTATTCTTG | 1937 | CAAGAATACCACGAAAGCA |
| NC_045512.2_19mer_winl_26308 | 26308 | 26326 | 735 | GCTTTCGTGGTATTCTTGC | 1938 | GCAAGAATACCACGAAAGC |
| NC_045512.2_19mer_winl_26309 | 26309 | 26327 | 736 | CTTTCGTGGTATTCTTGCT | 1939 | AGCAAGAATACCACGAAAG |
| NC_045512.2_19mer_winl_26310 | 26310 | 26328 | 737 | TTTCGTGGTATTCTTGCTA | 1940 | TAGCAAGAATACCACGAAA |
| NC_045512.2_19mer_winl_26311 | 26311 | 26329 | 738 | TTCGTGGTATTCTTGCTAG | 1941 | CTAGCAAGAATACCACGAA |
| NC_045512.2_19mer_win1_26312 | 26312 | 26330 | 739 | TCGTGGTATTCTTGCTAGT | 1942 | ACTAGCAAGAATACCACGA |
| NC_045512.2_19mer_win1_26332 | 26332 | 26350 | 740 | ACACTAGCCATCCTTACTG | 1943 | CAGTAAGGATGGCTAGTGT |
| NC_045512.2_19mer_win1_26333 | 26333 | 26351 | 741 | CACTAGCCATCCTTACTGC | 1944 | GCAGTAAGGATGGCTAGTG |
| NC_045512.2_19mer_win1_26334 | 26334 | 26352 | 742 | ACTAGCCATCCTTACTGCG | 1945 | CGCAGTAAGGATGGCTAGT |
| NC_045512.2_19mer_win1_26335 | 26335 | 26353 | 743 | CTAGCCATCCTTACTGCGC | 1946 | GCGCAGTAAGGATGGCTAG |
| NC_045512.2_19mer_win1_26336 | 26336 | 26354 | 744 | TAGCCATCCTTACTGCGCT | 1947 | AGCGCAGTAAGGATGGCTA |
| NC_045512.2_19mer_win1_26337 | 26337 | 26355 | 745 | AGCCATCCTTACTGCGCTT | 1948 | AAGCGCAGTAAGGATGGCT |
| NC_045512.2_19mer_win1_26338 | 26338 | 26356 | 746 | GCCATCCTTACTGCGCTTC | 1949 | GAAGCGCAGTAAGGATGGC |
| NC_045512.2_19mer_win1_26339 | 26339 | 26357 | 747 | CCATCCTTACTGCGCTTCG | 1950 | CGAAGCGCAGTAAGGATGG |
| NC_045512.2_19mer_win1_26340 | 26340 | 26358 | 748 | CATCCTTACTGCGCTTCGA | 1951 | TCGAAGCGCAGTAAGGATG |
| NC_045512.2_19mer_win1_26341 | 26341 | 26359 | 749 | ATCCTTACTGCGCTTCGAT | 1952 | ATCGAAGCGCAGTAAGGAT |
| NC_045512.2_19mer_win1_26342 | 26342 | 26360 | 750 | TCCTTACTGCGCTTCGATT | 1953 | AATCGAAGCGCAGTAAGGA |
| NC_045512.2_19mer_win1_26343 | 26343 | 26361 | 751 | CCTTACTGCGCTTCGATTG | 1954 | CAATCGAAGCGCAGTAAGG |
| NC_045512.2_19mer_win1_26344 | 26344 | 26362 | 752 | CTTACTGCGCTTCGATTGT | 1955 | ACAATCGAAGCGCAGTAAG |
| NC_045512.2_19mer_win1_26345 | 26345 | 26363 | 753 | TTACTGCGCTTCGATTGTG | 1956 | CACAATCGAAGCGCAGTAA |
| NC_045512.2_19mer_win1_26346 | 26346 | 26364 | 754 | TACTGCGCTTCGATTGTGT | 1957 | ACACAATCGAAGCGCAGTA |
| NC_045512.2_19mer_win1_26347 | 26347 | 26365 | 755 | ACTGCGCTTCGATTGTGTG | 1958 | CACACAATCGAAGCGCAGT |
| NC_045512.2_19mer_win1_26348 | 26348 | 26366 | 756 | CTGCGCTTCGATTGTGTGC | 1959 | GCACACAATCGAAGCGCAG |
| NC_045512.2_19mer_win1_26349 | 26349 | 26367 | 757 | TGCGCTTCGATTGTGTGCG | 1960 | CGCACACAATCGAAGCGCA |
| NC_045512.2_19mer_win1_26350 | 26350 | 26368 | 758 | GCGCTTCGATTGTGTGCGT | 1961 | ACGCACACAATCGAAGCGC |
| NC_045512.2_19mer_win1_26351 | 26351 | 26369 | 759 | CGCTTCGATTGTGTGCGTA | 1962 | TACGCACACAATCGAAGCG |
| NC_045512.2_19mer_win1_26352 | 26352 | 26370 | 760 | GCTTCGATTGTGTGCGTAC | 1963 | GTACGCACACAATCGAAGC |
| NC_045512.2_19mer_win1_26353 | 26353 | 26371 | 761 | CTTCGATTGTGTGCGTACT | 1964 | AGTACGCACACAATCGAAG |
| NC_045512.2_19mer_win1_26354 | 26354 | 26372 | 762 | TTCGATTGTGTGCGTACTG | 1965 | CAGTACGCACACAATCGAA |
| NC_045512.2_19mer_win1_26355 | 26355 | 26373 | 763 | TCGATTGTGTGCGTACTGC | 1966 | GCAGTACGCACACAATCGA |
| NC_045512.2_19mer_win1_26356 | 26356 | 26374 | 764 | CGATTGTGTGCGTACTGCT | 1967 | AGCAGTACGCACACAATCG |
| NC_045512.2_19mer_win1_26357 | 26357 | 26375 | 765 | GATTGTGTGCGTACTGCTG | 1968 | CAGCAGTACGCACACAATC |
| NC_045512.2_19mer_win1_26358 | 26358 | 26376 | 766 | ATTGTGTGCGTACTGCTGC | 1969 | GCAGCAGTACGCACACAAT |
| NC_045512.2_19mer_win1_26359 | 26359 | 26377 | 767 | TTGTGTGCGTACTGCTGCA | 1970 | TGCAGCAGTACGCACACAA |
| NC_045512.2_19mer_win1_26360 | 26360 | 26378 | 768 | TGTGTGCGTACTGCTGCAA | 1971 | TTGCAGCAGTACGCACACA |
| NC_045512.2_19mer_win1_26361 | 26361 | 26379 | 769 | GTGTGCGTACTGCTGCAAT | 1972 | ATTGCAGCAGTACGCACAC |
| NC_045512.2_19mer_win1_26362 | 26362 | 26380 | 770 | TGTGCGTACTGCTGCAATA | 1973 | TATTGCAGCAGTACGCACA |
| NC_045512.2_19mer_win1_26363 | 26363 | 26381 | 771 | GTGCGTACTGCTGCAATAT | 1974 | ATATTGCAGCAGTACGCAC |
| NC_045512.2_19mer_win1_26364 | 26364 | 26382 | 772 | TGCGTACTGCTGCAATATT | 1975 | AATATTGCAGCAGTACGCA |
| NC_045512.2_19mer_win1_26365 | 26365 | 26383 | 773 | GCGTACTGCTGCAATATTG | 1976 | CAATATTGCAGCAGTACGC |
| NC_045512.2_19mer_win1_26366 | 26366 | 26384 | 774 | CGTACTGCTGCAATATTGT | 1977 | ACAATATTGCAGCAGTACG |
| NC_045512.2_19mer_win1_26367 | 26367 | 26385 | 775 | GTACTGCTGCAATATTGTT | 1978 | AACAATATTGCAGCAGTAC |
| NC_045512.2_19mer_win1_26368 | 26368 | 26386 | 776 | TACTGCTGCAATATTGTTA | 1979 | TAACAATATTGCAGCAGTA |
| NC_045512.2_19mer_win1_26369 | 26369 | 26387 | 777 | ACTGCTGCAATATTGTTAA | 1980 | TTAACAATATTGCAGCAGT |
| NC_045512.2_19mer_win1_26370 | 26370 | 26388 | 778 | CTGCTGCAATATTGTTAAC | 1981 | GTTAACAATATTGCAGCAG |
| NC_045512.2_19mer_win1_26371 | 26371 | 26389 | 779 | TGCTGCAATATTGTTAACG | 1982 | CGTTAACAATATTGCAGCA |
| NC_045512.2_19mer_win1_26372 | 26372 | 26390 | 780 | GCTGCAATATTGTTAACGT | 1983 | ACGTTAACAATATTGCAGC |
| NC_045512.2_19mer_win1_26373 | 26373 | 26391 | 781 | CTGCAATATTGTTAACGTG | 1984 | CACGTTAACAATATTGCAG |
| NC_045512.2_19mer_win1_26374 | 26374 | 26392 | 782 | TGCAATATTGTTAACGTGA | 1985 | TCACGTTAACAATATTGCA |
| NC_045512.2_19mer_win1_26375 | 26375 | 26393 | 783 | GCAATATTGTTAACGTGAG | 1986 | CTCACGTTAACAATATTGC |
| NC_045512.2_19mer_win1_26376 | 26376 | 26394 | 784 | CAATATTGTTAACGTGAGT | 1987 | ACTCACGTTAACAATATTG |
| NC_045512.2_19mer_win1_26450 | 26450 | 26468 | 785 | GAGTTCCTGATCTTCTGGT | 1988 | ACCAGAAGATCAGGAACTC |
| NC_045512.2_19mer_win1_26451 | 26451 | 26469 | 786 | AGTTCCTGATCTTCTGGTC | 1989 | GACCAGAAGATCAGGAACT |
| NC_045512.2_19mer_win1_26452 | 26452 | 26470 | 787 | GTTCCTGATCTTCTGGTCT | 1990 | AGACCAGAAGATCAGGAAC |
| NC_045512.2_19mer_win1_26453 | 26453 | 26471 | 788 | TTCCTGATCTTCTGGTCTA | 1991 | TAGACCAGAAGATCAGGAA |
| NC_045512.2_19mer_win1_26454 | 26454 | 26472 | 789 | TCCTGATCTTCTGGTCTAA | 1992 | TTAGACCAGAAGATCAGGA |
| NC_045512.2_19mer_win1_26455 | 26455 | 26473 | 790 | CCTGATCTTCTGGTCTAAA | 1993 | TTTAGACCAGAAGATCAGG |
| NC_045512.2_19mer_win1_26456 | 26456 | 26474 | 791 | CTGATCTTCTGGTCTAAAC | 1994 | GTTTAGACCAGAAGATCAG |
| NC_045512.2_19mer_win1_26457 | 26457 | 26475 | 792 | TGATCTTCTGGTCTAAACG | 1995 | CGTTTAGACCAGAAGATCA |
| NC_045512.2_19mer_win1_26458 | 26458 | 26476 | 793 | GATCTTCTGGTCTAAACGA | 1996 | TCGTTTAGACCAGAAGATC |
| NC_045512.2_19mer_win1_26459 | 26459 | 26477 | 794 | ATCTTCTGGTCTAAACGAA | 1997 | TTCGTTTAGACCAGAAGAT |
| NC_045512.2_19mer_win1_26460 | 26460 | 26478 | 795 | TCTTCTGGTCTAAACGAAC | 1998 | GTTCGTTTAGACCAGAAGA |
| NC_045512.2_19mer_win1_26461 | 26461 | 26479 | 796 | CTTCTGGTCTAAACGAACT | 1999 | AGTTCGTTTAGACCAGAAG |
| NC_045512.2_19mer_win1_26462 | 26462 | 26480 | 797 | TTCTGGTCTAAACGAACTA | 2000 | TAGTTCGTTTAGACCAGAA |
| NC_045512.2_19mer_win1_26463 | 26463 | 26481 | 798 | TCTGGTCTAAACGAACTAA | 2001 | TTAGTTCGTTTAGACCAGA |
| NC_045512.2_19mer_win1_26574 | 26574 | 26592 | 799 | GAACAATGGAACCTAGTAA | 2002 | TTACTAGGTTCCATTGTTC |
| NC_045512.2_19mer_win1_26575 | 26575 | 26593 | 800 | AACAATGGAACCTAGTAAT | 2003 | ATTACTAGGTTCCATTGTT |
| NC_045512.2_19mer_win1_26576 | 26576 | 26594 | 801 | ACAATGGAACCTAGTAATA | 2004 | TATTACTAGGTTCCATTGT |
| NC_045512.2_19mer_win1_26577 | 26577 | 26595 | 802 | CAATGGAACCTAGTAATAG | 2005 | CTATTACTAGGTTCCATTG |
| NC_045512.2_19mer_win1_26578 | 26578 | 26596 | 803 | AATGGAACCTAGTAATAGG | 2006 | CCTATTACTAGGTTCCATT |
| NC_045512.2_19mer_win1_26579 | 26579 | 26597 | 804 | ATGGAACCTAGTAATAGGT | 2007 | ACCTATTACTAGGTTCCAT |
| NC_045512.2_19mer_win1_26580 | 26580 | 26598 | 805 | TGGAACCTAGTAATAGGTT | 2008 | AACCTATTACTAGGTTCCA |
| NC_045512.2_19mer_win1_26581 | 26581 | 26599 | 806 | GGAACCTAGTAATAGGTTT | 2009 | AAACCTATTACTAGGTTCC |
| NC_045512.2_19mer_win1_26582 | 26582 | 26600 | 807 | GAACCTAGTAATAGGTTTC | 2010 | GAAACCTATTACTAGGTTC |
| NC_045512.2_19mer_win1_27033 | 27033 | 27051 | 808 | GCTACATCACGAACGCTTT | 2011 | AAAGCGTTCGTGATGTAGC |
| NC_045512.2_19mer_win1_27034 | 27034 | 27052 | 809 | CTACATCACGAACGCTTTC | 2012 | GAAAGCGTTCGTGATGTAG |
| NC_045512.2_19mer_win1_27035 | 27035 | 27053 | 810 | TACATCACGAACGCTTTCT | 2013 | AGAAAGCGTTCGTGATGTA |
| NC_045512.2_19mer_win1_27036 | 27036 | 27054 | 811 | ACATCACGAACGCTTTCTT | 2014 | AAGAAAGCGTTCGTGATGT |
| NC_045512.2_19mer_win1_27037 | 27037 | 27055 | 812 | CATCACGAACGCTTTCTTA | 2015 | TAAGAAAGCGTTCGTGATG |
| NC_045512.2_19mer_win1_27038 | 27038 | 27056 | 813 | ATCACGAACGCTTTCTTAT | 2016 | ATAAGAAAGCGTTCGTGAT |
| NC_045512.2_19mer_win1_27039 | 27039 | 27057 | 814 | TCACGAACGCTTTCTTATT | 2017 | AATAAGAAAGCGTTCGTGA |
| NC_045512.2_19mer_win1_27040 | 27040 | 27058 | 815 | CACGAACGCTTTCTTATTA | 2018 | TAATAAGAAAGCGTTCGTG |
| NC_045512.2_19mer_win1_27041 | 27041 | 27059 | 816 | ACGAACGCTTTCTTATTAC | 2019 | GTAATAAGAAAGCGTTCGT |
| NC_045512.2_19mer_win1_27042 | 27042 | 27060 | 817 | CGAACGCTTTCTTATTACA | 2020 | TGTAATAAGAAAGCGTTCG |
| NC_045512.2_19mer_win1_27043 | 27043 | 27061 | 818 | GAACGCTTTCTTATTACAA | 2021 | TTGTAATAAGAAAGCGTTC |
| NC_045512.2_19mer_win1_27044 | 27044 | 27062 | 819 | AACGCTTTCTTATTACAAA | 2022 | TTTGTAATAAGAAAGCGTT |
| NC_045512.2_19mer_win1_27045 | 27045 | 27063 | 820 | ACGCTTTCTTATTACAAAT | 2023 | ATTTGTAATAAGAAAGCGT |
| NC_045512.2_19mer_win1_27046 | 27046 | 27064 | 821 | CGCTTTCTTATTACAAATT | 2024 | AATTTGTAATAAGAAAGCG |
| NC_045512.2_19mer_win1_27093 | 27093 | 27111 | 822 | TCAGGTTTTGCTGCATACA | 2025 | TGTATGCAGCAAAACCTGA |
| NC_045512.2_19mer_win1_27183 | 27183 | 27201 | 823 | GTACAGTAAGTGACAACAG | 2026 | CTGTTGTCACTTACTGTAC |
| NC_045512.2_19mer_win1_27184 | 27184 | 27202 | 824 | TACAGTAAGTGACAACAGA | 2027 | TCTGTTGTCACTTACTGTA |
| NC_045512.2_19mer_win1_27185 | 27185 | 27203 | 825 | ACAGTAAGTGACAACAGAT | 2028 | ATCTGTTGTCACTTACTGT |
| NC_045512.2_19mer_win1_27186 | 27186 | 27204 | 826 | CAGTAAGTGACAACAGATG | 2029 | CATCTGTTGTCACTTACTG |
| NC_045512.2_19mer_win1_27187 | 27187 | 27205 | 827 | AGTAAGTGACAACAGATGT | 2030 | ACATCTGTTGTCACTTACT |
| NC_045512.2_19mer_win1_27188 | 27188 | 27206 | 828 | GTAAGTGACAACAGATGTT | 2031 | AACATCTGTTGTCACTTAC |
| NC_045512.2_19mer_win1_27189 | 27189 | 27207 | 829 | TAAGTGACAACAGATGTTT | 2032 | AAACATCTGTTGTCACTTA |
| NC_045512.2_19mer_win1_27190 | 27190 | 27208 | 830 | AAGTGACAACAGATGTTTC | 2033 | GAAACATCTGTTGTCACTT |
| NC_045512.2_19mer_win1_27191 | 27191 | 27209 | 831 | AGTGACAACAGATGTTTCA | 2034 | TGAAACATCTGTTGTCACT |
| NC_045512.2_19mer_win1_27192 | 27192 | 27210 | 832 | GTGACAACAGATGTTTCAT | 2035 | ATGAAACATCTGTTGTCAC |
| NC_045512.2_19mer_win1_27193 | 27193 | 27211 | 833 | TGACAACAGATGTTTCATC | 2036 | GATGAAACATCTGTTGTCA |
| NC_045512.2_19mer_win1_27194 | 27194 | 27212 | 834 | GACAACAGATGTTTCATCT | 2037 | AGATGAAACATCTGTTGTC |
| NC_045512.2_19mer_win1_27382 | 27382 | 27400 | 835 | GATTAAACGAACATGAAAA | 2038 | TTTTCATGTTCGTTTAATC |
| NC_045512.2_19mer_win1_27383 | 27383 | 27401 | 836 | ATTAAACGAACATGAAAAT | 2039 | ATTTTCATGTTCGTTTAAT |
| NC_045512.2_19mer_win1_27384 | 27384 | 27402 | 837 | TTAAACGAACATGAAAATT | 2040 | AATTTTCATGTTCGTTTAA |
| NC_045512.2_19mer_win1_27385 | 27385 | 27403 | 838 | TAAACGAACATGAAAATTA | 2041 | TAATTTTCATGTTCGTTTA |
| NC_045512.2_19mer_win1_27386 | 27386 | 27404 | 839 | AAACGAACATGAAAATTAT | 2042 | ATAATTTTCATGTTCGTTT |
| NC_045512.2_19mer_win1_27387 | 27387 | 27405 | 840 | AACGAACATGAAAATTATT | 2043 | AATAATTTTCATGTTCGTT |
| NC_045512.2_19mer_win1_27388 | 27388 | 27406 | 841 | ACGAACATGAAAATTATTC | 2044 | GAATAATTTTCATGTTCGT |
| NC_045512.2_19mer_win1_27389 | 27389 | 27407 | 842 | CGAACATGAAAATTATTCT | 2045 | AGAATAATTTTCATGTTCG |
| NC_045512.2_19mer_win1_27511 | 27511 | 27529 | 843 | TACGAGGGCAATTCACCAT | 2046 | ATGGTGAATTGCCCTCGTA |
| NC_045512.2_19mer_win1_27512 | 27512 | 27530 | 844 | ACGAGGGCAATTCACCATT | 2047 | AATGGTGAATTGCCCTCGT |
| NC_045512.2_19mer_win1_27513 | 27513 | 27531 | 845 | CGAGGGCAATTCACCATTT | 2048 | AAATGGTGAATTGCCCTCG |
| NC_045512.2_19mer_win1_27514 | 27514 | 27532 | 846 | GAGGGCAATTCACCATTTC | 2049 | GAAATGGTGAATTGCCCTC |
| NC_045512.2_19mer_win1_27515 | 27515 | 27533 | 847 | AGGGCAATTCACCATTTCA | 2050 | TGAAATGGTGAATTGCCCT |
| NC_045512.2_19mer_win1_27771 | 27771 | 27789 | 848 | TTAATTGACTTCTATTTGT | 2051 | ACAAATAGAAGTCAATTAA |
| NC_045512.2_19mer_win1_27772 | 27772 | 27790 | 849 | TAATTGACTTCTATTTGTG | 2052 | CACAAATAGAAGTCAATTA |
| NC_045512.2_19mer_win1_27773 | 27773 | 27791 | 850 | AATTGACTTCTATTTGTGC | 2053 | GCACAAATAGAAGTCAATT |
| NC_045512.2_19mer_win1_27774 | 27774 | 27792 | 851 | ATTGACTTCTATTTGTGCT | 2054 | AGCACAAATAGAAGTCAAT |
| NC_045512.2_19mer_win1_27775 | 27775 | 27793 | 852 | TTGACTTCTATTTGTGCTT | 2055 | AAGCACAAATAGAAGTCAA |
| NC_045512.2_19mer_win1_27776 | 27776 | 27794 | 853 | TGACTTCTATTTGTGCTTT | 2056 | AAAGCACAAATAGAAGTCA |
| NC_045512.2_19mer_win1_27777 | 27777 | 27795 | 854 | GACTTCTATTTGTGCTTTT | 2057 | AAAAGCACAAATAGAAGTC |
| NC_045512.2_19mer_win1_27778 | 27778 | 27796 | 855 | ACTTCTATTTGTGCTTTTT | 2058 | AAAAAGCACAAATAGAAGT |
| NC_045512.2_19mer_win1_27779 | 27779 | 27797 | 856 | CTTCTATTTGTGCTTTTTA | 2059 | TAAAAAGCACAAATAGAAG |
| NC_045512.2_19mer_win1_27780 | 27780 | 27798 | 857 | TTCTATTTGTGCTTTTTAG | 2060 | CTAAAAAGCACAAATAGAA |
| NC_045512.2_19mer_win1_27781 | 27781 | 27799 | 858 | TCTATTTGTGCTTTTTAGC | 2061 | GCTAAAAAGCACAAATAGA |
| NC_045512.2_19mer_win1_27782 | 27782 | 27800 | 859 | CTATTTGTGCTTTTTAGCC | 2062 | GGCTAAAAAGCACAAATAG |
| NC_045512.2_19mer_win1_27783 | 27783 | 27801 | 860 | TATTTGTGCTTTTTAGCCT | 2063 | AGGCTAAAAAGCACAAATA |
| NC_045512.2_19mer_win1_27784 | 27784 | 27802 | 861 | ATTTGTGCTTTTTAGCCTT | 2064 | AAGGCTAAAAAGCACAAAT |
| NC_045512.2_19mer_win1_27785 | 27785 | 27803 | 862 | TTTGTGCTTTTTAGCCTTT | 2065 | AAAGGCTAAAAAGCACAAA |
| NC_045512.2_19mer_win1_27786 | 27786 | 27804 | 863 | TTGTGCTTTTTAGCCTTTC | 2066 | GAAAGGCTAAAAAGCACAA |
| NC_045512.2_19mer_win1_27787 | 27787 | 27805 | 864 | TGTGCTTTTTAGCCTTTCT | 2067 | AGAAAGGCTAAAAAGCACA |
| NC_045512.2_19mer_win1_27788 | 27788 | 27806 | 865 | GTGCTTTTTAGCCTTTCTG | 2068 | CAGAAAGGCTAAAAAGCAC |
| NC_045512.2_19mer_win1_27789 | 27789 | 27807 | 866 | TGCTTTTTAGCCTTTCTGC | 2069 | GCAGAAAGGCTAAAAAGCA |
| NC_045512.2_19mer_win1_27790 | 27790 | 27808 | 867 | GCTTTTTAGCCTTTCTGCT | 2070 | AGCAGAAAGGCTAAAAAGC |
| NC_045512.2_19mer_win1_27791 | 27791 | 27809 | 868 | CTTTTTAGCCTTTCTGCTA | 2071 | TAGCAGAAAGGCTAAAAAG |
| NC_045512.2_19mer_win1_27792 | 27792 | 27810 | 869 | TTTTTAGCCTTTCTGCTAT | 2072 | ATAGCAGAAAGGCTAAAAA |
| NC_045512.2_19mer_win1_27793 | 27793 | 27811 | 870 | TTTTAGCCTTTCTGCTATT | 2073 | AATAGCAGAAAGGCTAAAA |
| NC_045512.2_19mer_win1_27794 | 27794 | 27812 | 871 | TTTAGCCTTTCTGCTATTC | 2074 | GAATAGCAGAAAGGCTAAA |
| NC_045512.2_19mer_win1_27795 | 27795 | 27813 | 872 | TTAGCCTTTCTGCTATTCC | 2075 | GGAATAGCAGAAAGGCTAA |
| NC_045512.2_19mer_win1_27796 | 27796 | 27814 | 873 | TAGCCTTTCTGCTATTCCT | 2076 | AGGAATAGCAGAAAGGCTA |
| NC_045512.2_19mer_win1_27797 | 27797 | 27815 | 874 | AGCCTTTCTGCTATTCCTT | 2077 | AAGGAATAGCAGAAAGGCT |
| NC_045512.2_19mer_win1_27798 | 27798 | 27816 | 875 | GCCTTTCTGCTATTCCTTG | 2078 | CAAGGAATAGCAGAAAGGC |
| NC_045512.2_19mer_win1_27799 | 27799 | 27817 | 876 | CCTTTCTGCTATTCCTTGT | 2079 | ACAAGGAATAGCAGAAAGG |
| NC_045512.2_19mer_win1_27800 | 27800 | 27818 | 877 | CTTTCTGCTATTCCTTGTT | 2080 | AACAAGGAATAGCAGAAAG |
| NC_045512.2_19mer_win1_28270 | 28270 | 28288 | 878 | TAAAATGTCTGATAATGGA | 2081 | TCCATTATCAGACATTTTA |
| NC_045512.2_19mer_win1_28271 | 28271 | 28289 | 879 | AAAATGTCTGATAATGGAC | 2082 | GTCCATTATCAGACATTTT |
| NC_045512.2_19mer_win1_28272 | 28272 | 28290 | 880 | AAATGTCTGATAATGGACC | 2083 | GGTCCATTATCAGACATTT |
| NC_045512.2_19mer_win1_28273 | 28273 | 28291 | 881 | AATGTCTGATAATGGACCC | 2084 | GGGTCCATTATCAGACATT |
| NC_045512.2_19mer_win1_28274 | 28274 | 28292 | 882 | ATGTCTGATAATGGACCCC | 2085 | GGGGTCCATTATCAGACAT |
| NC_045512.2_19mer_win1_28275 | 28275 | 28293 | 883 | TGTCTGATAATGGACCCCA | 2086 | TGGGGTCCATTATCAGACA |
| NC_045512.2_19mer_win1_28276 | 28276 | 28294 | 884 | GTCTGATAATGGACCCCAA | 2087 | TTGGGGTCCATTATCAGAC |
| NC_045512.2_19mer_win1_28277 | 28277 | 28295 | 885 | TCTGATAATGGACCCCAAA | 2088 | TTTGGGGTCCATTATCAGA |
| NC_045512.2_19mer_win1_28278 | 28278 | 28296 | 886 | CTGATAATGGACCCCAAAA | 2089 | TTTTGGGGTCCATTATCAG |
| NC_045512.2_19mer_win1_28397 | 28397 | 28415 | 887 | CCCCAAGGTTTACCCAATA | 2090 | TATTGGGTAAACCTTGGGG |
| NC_045512.2_19mer_win1_28398 | 28398 | 28416 | 888 | CCCAAGGTTTACCCAATAA | 2091 | TTATTGGGTAAACCTTGGG |
| NC_045512.2_19mer_win1_28399 | 28399 | 28417 | 889 | CCAAGGTTTACCCAATAAT | 2092 | ATTATTGGGTAAACCTTGG |
| NC_045512.2_19mer_win1_28400 | 28400 | 28418 | 890 | CAAGGTTTACCCAATAATA | 2093 | TATTATTGGGTAAACCTTG |
| NC_045512.2_19mer_win1_28401 | 28401 | 28419 | 891 | AAGGTTTACCCAATAATAC | 2094 | GTATTATTGGGTAAACCTT |
| NC_045512.2_19mer_win1_28402 | 28402 | 28420 | 892 | AGGTTTACCCAATAATACT | 2095 | AGTATTATTGGGTAAACCT |
| NC_045512.2_19mer_win1_28403 | 28403 | 28421 | 893 | GGTTTACCCAATAATACTG | 2096 | CAGTATTATTGGGTAAACC |
| NC_045512.2_19mer_win1_28404 | 28404 | 28422 | 894 | GTTTACCCAATAATACTGC | 2097 | GCAGTATTATTGGGTAAAC |
| NC_045512.2_19mer_win1_28405 | 28405 | 28423 | 895 | TTTACCCAATAATACTGCG | 2098 | CGCAGTATTATTGGGTAAA |
| NC_045512.2_19mer_win1_28406 | 28406 | 28424 | 896 | TTACCCAATAATACTGCGT | 2099 | ACGCAGTATTATTGGGTAA |
| NC_045512.2_19mer_win1_28407 | 28407 | 28425 | 897 | TACCCAATAATACTGCGTC | 2100 | GACGCAGTATTATTGGGTA |
| NC_045512.2_19mer_win1_28408 | 28408 | 28426 | 898 | ACCCAATAATACTGCGTCT | 2101 | AGACGCAGTATTATTGGGT |
| NC_045512.2_19mer_win1_28409 | 28409 | 28427 | 899 | CCCAATAATACTGCGTCTT | 2102 | AAGACGCAGTATTATTGGG |
| NC_045512.2_19mer_win1_28410 | 28410 | 28428 | 900 | CCAATAATACTGCGTCTTG | 2103 | CAAGACGCAGTATTATTGG |
| NC_045512.2_19mer_win1_28411 | 28411 | 28429 | 901 | CAATAATACTGCGTCTTGG | 2104 | CCAAGACGCAGTATTATTG |
| NC_045512.2_19mer_win1_28412 | 28412 | 28430 | 902 | AATAATACTGCGTCTTGGT | 2105 | ACCAAGACGCAGTATTATT |
| NC_045512.2_19mer_win1_28413 | 28413 | 28431 | 903 | ATAATACTGCGTCTTGGTT | 2106 | AACCAAGACGCAGTATTAT |
| NC_045512.2_19mer_win1_28414 | 28414 | 28432 | 904 | TAATACTGCGTCTTGGTTC | 2107 | GAACCAAGACGCAGTATTA |
| NC_045512.2_19mer_win1_28415 | 28415 | 28433 | 905 | AATACTGCGTCTTGGTTCA | 2108 | TGAACCAAGACGCAGTATT |
| NC_045512.2_19mer_win1_28416 | 28416 | 28434 | 906 | ATACTGCGTCTTGGTTCAC | 2109 | GTGAACCAAGACGCAGTAT |
| NC_045512.2_19mer_win1_28513 | 28513 | 28531 | 907 | AGATGACCAAATTGGCTAC | 2110 | GTAGCCAATTTGGTCATCT |
| NC_045512.2_19mer_win1_28514 | 28514 | 28532 | 908 | GATGACCAAATTGGCTACT | 2111 | AGTAGCCAATTTGGTCATC |
| NC_045512.2_19mer_win1_28515 | 28515 | 28533 | 909 | ATGACCAAATTGGCTACTA | 2112 | TAGTAGCCAATTTGGTCAT |
| NC_045512.2_19mer_win1_28516 | 28516 | 28534 | 910 | TGACCAAATTGGCTACTAC | 2113 | GTAGTAGCCAATTTGGTCA |
| NC_045512.2_19mer_win1_28517 | 28517 | 28535 | 911 | GACCAAATTGGCTACTACC | 2114 | GGTAGTAGCCAATTTGGTC |
| NC_045512.2_19mer_win1_28518 | 28518 | 28536 | 912 | ACCAAATTGGCTACTACCG | 2115 | CGGTAGTAGCCAATTTGGT |
| NC_045512.2_19mer_win1_28519 | 28519 | 28537 | 913 | CCAAATTGGCTACTACCGA | 2116 | TCGGTAGTAGCCAATTTGG |
| NC_045512.2_19mer_win1_28520 | 28520 | 28538 | 914 | CAAATTGGCTACTACCGAA | 2117 | TTCGGTAGTAGCCAATTTG |
| NC_045512.2_19mer_win1_28521 | 28521 | 28539 | 915 | AAATTGGCTACTACCGAAG | 2118 | CTTCGGTAGTAGCCAATTT |
| NC_045512.2_19mer_win1_28522 | 28522 | 28540 | 916 | AATTGGCTACTACCGAAGA | 2119 | TCTTCGGTAGTAGCCAATT |
| NC_045512.2_19mer_win1_28523 | 28523 | 28541 | 917 | ATTGGCTACTACCGAAGAG | 2120 | CTCTTCGGTAGTAGCCAAT |
| NC_045512.2_19mer_win1_28524 | 28524 | 28542 | 918 | TTGGCTACTACCGAAGAGC | 2121 | GCTCTTCGGTAGTAGCCAA |
| NC_045512.2_19mer_win1_28525 | 28525 | 28543 | 919 | TGGCTACTACCGAAGAGCT | 2122 | AGCTCTTCGGTAGTAGCCA |
| NC_045512.2_19mer_win1_28526 | 28526 | 28544 | 920 | GGCTACTACCGAAGAGCTA | 2123 | TAGCTCTTCGGTAGTAGCC |
| NC_045512.2_19mer_win1_28527 | 28527 | 28545 | 921 | GCTACTACCGAAGAGCTAC | 2124 | GTAGCTCTTCGGTAGTAGC |
| NC_045512.2_19mer_win1_28528 | 28528 | 28546 | 922 | CTACTACCGAAGAGCTACC | 2125 | GGTAGCTCTTCGGTAGTAG |
| NC_045512.2_19mer_win1_28673 | 28673 | 28691 | 923 | GCAACTGAGGGAGCCTTGA | 2126 | TCAAGGCTCCCTCAGTTGC |
| NC_045512.2_19mer_win1_28674 | 28674 | 28692 | 924 | CAACTGAGGGAGCCTTGAA | 2127 | TTCAAGGCTCCCTCAGTTG |
| NC_045512.2_19mer_win1_28706 | 28706 | 28724 | 925 | CACATTGGCACCCGCAATC | 2128 | GATTGCGGGTGCCAATGTG |
| NC_045512.2_19mer_win1_28707 | 28707 | 28725 | 926 | ACATTGGCACCCGCAATCC | 2129 | GGATTGCGGGTGCCAATGT |
| NC_045512.2_19mer_win1_28708 | 28708 | 28726 | 927 | CATTGGCACCCGCAATCCT | 2130 | AGGATTGCGGGTGCCAATG |
| NC_045512.2_19mer_win1_28744 | 28744 | 28762 | 928 | CGTGCTACAACTTCCTCAA | 2131 | TTGAGGAAGTTGTAGCACG |
| NC_045512.2_19mer_win1_28745 | 28745 | 28763 | 929 | GTGCTACAACTTCCTCAAG | 2132 | CTTGAGGAAGTTGTAGCAC |
| NC_045512.2_19mer_win1_28746 | 28746 | 28764 | 930 | TGCTACAACTTCCTCAAGG | 2133 | CCTTGAGGAAGTTGTAGCA |
| NC_045512.2_19mer_win1_28747 | 28747 | 28765 | 931 | GCTACAACTTCCTCAAGGA | 2134 | TCCTTGAGGAAGTTGTAGC |
| NC_045512.2_19mer_win1_28748 | 28748 | 28766 | 932 | CTACAACTTCCTCAAGGAA | 2135 | TTCCTTGAGGAAGTTGTAG |
| NC_045512.2_19mer_win1_28749 | 28749 | 28767 | 933 | TACAACTTCCTCAAGGAAC | 2136 | GTTCCTTGAGGAAGTTGTA |
| NC_045512.2_19mer_win1_28750 | 28750 | 28768 | 934 | ACAACTTCCTCAAGGAACA | 2137 | TGTTCCTTGAGGAAGTTGT |
| NC_045512.2_19mer_win1_28751 | 28751 | 28769 | 935 | CAACTTCCTCAAGGAACAA | 2138 | TTGTTCCTTGAGGAAGTTG |
| NC_045512.2_19mer_win1_28752 | 28752 | 28770 | 936 | AACTTCCTCAAGGAACAAC | 2139 | GTTGTTCCTTGAGGAAGTT |
| NC_045512.2_19mer_win1_28753 | 28753 | 28771 | 937 | ACTTCCTCAAGGAACAACA | 2140 | TGTTGTTCCTTGAGGAAGT |
| NC_045512.2_19mer_win1_28754 | 28754 | 28772 | 938 | CTTCCTCAAGGAACAACAT | 2141 | ATGTTGTTCCTTGAGGAAG |
| NC_045512.2_19mer_win1_28755 | 28755 | 28773 | 939 | TTCCTCAAGGAACAACATT | 2142 | AATGTTGTTCCTTGAGGAA |
| NC_045512.2_19mer_win1_28756 | 28756 | 28774 | 940 | TCCTCAAGGAACAACATTG | 2143 | CAATGTTGTTCCTTGAGGA |
| NC_045512.2_19mer_win1_28757 | 28757 | 28775 | 941 | CCTCAAGGAACAACATTGC | 2144 | GCAATGTTGTTCCTTGAGG |
| NC_045512.2_19mer_win1_28758 | 28758 | 28776 | 942 | CTCAAGGAACAACATTGCC | 2145 | GGCAATGTTGTTCCTTGAG |
| NC_045512.2_19mer_win1_28759 | 28759 | 28777 | 943 | TCAAGGAACAACATTGCCA | 2146 | TGGCAATGTTGTTCCTTGA |
| NC_045512.2_19mer_win1_28760 | 28760 | 28778 | 944 | CAAGGAACAACATTGCCAA | 2147 | TTGGCAATGTTGTTCCTTG |
| NC_045512.2_19mer_win1_28761 | 28761 | 28779 | 945 | AAGGAACAACATTGCCAAA | 2148 | TTTGGCAATGTTGTTCCTT |
| NC_045512.2_19mer_win1_28762 | 28762 | 28780 | 946 | AGGAACAACATTGCCAAAA | 2149 | TTTTGGCAATGTTGTTCCT |
| NC_045512.2_19mer_win1_28763 | 28763 | 28781 | 947 | GGAACAACATTGCCAAAAG | 2150 | CTTTTGGCAATGTTGTTCC |
| NC_045512.2_19mer_win1_28764 | 28764 | 28782 | 948 | GAACAACATTGCCAAAAGG | 2151 | CCTTTTGGCAATGTTGTTC |
| NC_045512.2_19mer_win1_28765 | 28765 | 28783 | 949 | AACAACATTGCCAAAAGGC | 2152 | GCCTTTTGGCAATGTTGTT |
| NC_045512.2_19mer_win1_28766 | 28766 | 28784 | 950 | ACAACATTGCCAAAAGGCT | 2153 | AGCCTTTTGGCAATGTTGT |
| NC_045512.2_19mer_win1_28767 | 28767 | 28785 | 951 | CAACATTGCCAAAAGGCTT | 2154 | AAGCCTTTTGGCAATGTTG |
| NC_045512.2_19mer_win1_28768 | 28768 | 28786 | 952 | AACATTGCCAAAAGGCTTC | 2155 | GAAGCCTTTTGGCAATGTT |
| NC_045512.2_19mer_win1_28769 | 28769 | 28787 | 953 | ACATTGCCAAAAGGCTTCT | 2156 | AGAAGCCTTTTGGCAATGT |
| NC_045512.2_19mer_win1_28770 | 28770 | 28788 | 954 | CATTGCCAAAAGGCTTCTA | 2157 | TAGAAGCCTTTTGGCAATG |
| NC_045512.2_19mer_win1_28771 | 28771 | 28789 | 955 | ATTGCCAAAAGGCTTCTAC | 2158 | GTAGAAGCCTTTTGGCAAT |
| NC_045512.2_19mer_win1_28772 | 28772 | 28790 | 956 | TTGCCAAAAGGCTTCTACG | 2159 | CGTAGAAGCCTTTTGGCAA |
| NC_045512.2_19mer_win1_28773 | 28773 | 28791 | 957 | TGCCAAAAGGCTTCTACGC | 2160 | GCGTAGAAGCCTTTTGGCA |
| NC_045512.2_19mer_win1_28774 | 28774 | 28792 | 958 | GCCAAAAGGCTTCTACGCA | 2161 | TGCGTAGAAGCCTTTTGGC |
| NC_045512.2_19mer_win1_28775 | 28775 | 28793 | 959 | CCAAAAGGCTTCTACGCAG | 2162 | CTGCGTAGAAGCCTTTTGG |
| NC_045512.2_19mer_win1_28776 | 28776 | 28794 | 960 | CAAAAGGCTTCTACGCAGA | 2163 | TCTGCGTAGAAGCCTTTTG |
| NC_045512.2_19mer_win1_28799 | 28799 | 28817 | 961 | AGCAGAGGCGGCAGTCAAG | 2164 | CTTGACTGCCGCCTCTGCT |
| NC_045512.2_19mer_win1_28800 | 28800 | 28818 | 962 | GCAGAGGCGGCAGTCAAGC | 2165 | GCTTGACTGCCGCCTCTGC |
| NC_045512.2_19mer_win1_28801 | 28801 | 28819 | 963 | CAGAGGCGGCAGTCAAGCC | 2166 | GGCTTGACTGCCGCCTCTG |
| NC_045512.2_19mer_win1_28802 | 28802 | 28820 | 964 | AGAGGCGGCAGTCAAGCCT | 2167 | AGGCTTGACTGCCGCCTCT |
| NC_045512.2_19mer_win1_28803 | 28803 | 28821 | 965 | GAGGCGGCAGTCAAGCCTC | 2168 | GAGGCTTGACTGCCGCCTC |
| NC_045512.2_19mer_win1_28804 | 28804 | 28822 | 966 | AGGCGGCAGTCAAGCCTCT | 2169 | AGAGGCTTGACTGCCGCCT |
| NC_045512.2_19mer_win1_28805 | 28805 | 28823 | 967 | GGCGGCAGTCAAGCCTCTT | 2170 | AAGAGGCTTGACTGCCGCC |
| NC_045512.2_19mer_win1_28806 | 28806 | 28824 | 968 | GCGGCAGTCAAGCCTCTTC | 2171 | GAAGAGGCTTGACTGCCGC |
| NC_045512.2_19mer_win1_28807 | 28807 | 28825 | 969 | CGGCAGTCAAGCCTCTTCT | 2172 | AGAAGAGGCTTGACTGCCG |
| NC_045512.2_19mer_win1_28808 | 28808 | 28826 | 970 | GGCAGTCAAGCCTCTTCTC | 2173 | GAGAAGAGGCTTGACTGCC |
| NC_045512.2_19mer_win1_28809 | 28809 | 28827 | 971 | GCAGTCAAGCCTCTTCTCG | 2174 | CGAGAAGAGGCTTGACTGC |
| NC_045512.2_19mer_win1_28946 | 28946 | 28964 | 972 | GACAGATTGAACCAGCTTG | 2175 | CAAGCTGGTTCAATCTGTC |
| NC_045512.2_19mer_win1_28947 | 28947 | 28965 | 973 | ACAGATTGAACCAGCTTGA | 2176 | TCAAGCTGGTTCAATCTGT |
| NC_045512.2_19mer_win1_28948 | 28948 | 28966 | 974 | CAGATTGAACCAGCTTGAG | 2177 | CTCAAGCTGGTTCAATCTG |
| NC_045512.2_19mer_win1_28949 | 28949 | 28967 | 975 | AGATTGAACCAGCTTGAGA | 2178 | TCTCAAGCTGGTTCAATCT |
| NC_045512.2_19mer_win1_28950 | 28950 | 28968 | 976 | GATTGAACCAGCTTGAGAG | 2179 | CTCTCAAGCTGGTTCAATC |
| NC_045512.2_19mer_win1_28951 | 28951 | 28969 | 977 | ATTGAACCAGCTTGAGAGC | 2180 | GCTCTCAAGCTGGTTCAAT |
| NC_045512.2_19mer_win1_28952 | 28952 | 28970 | 978 | TTGAACCAGCTTGAGAGCA | 2181 | TGCTCTCAAGCTGGTTCAA |
| NC_045512.2_19mer_win1_28953 | 28953 | 28971 | 979 | TGAACCAGCTTGAGAGCAA | 2182 | TTGCTCTCAAGCTGGTTCA |
| NC_045512.2_19mer_win1_28954 | 28954 | 28972 | 980 | GAACCAGCTTGAGAGCAAA | 2183 | TTTGCTCTCAAGCTGGTTC |
| NC_045512.2_19mer_win1_28976 | 28976 | 28994 | 981 | TCTGGTAAAGGCCAACAAC | 2184 | GTTGTTGGCCTTTACCAGA |
| NC_045512.2_19mer_win1_28977 | 28977 | 28995 | 982 | CTGGTAAAGGCCAACAACA | 2185 | TGTTGTTGGCCTTTACCAG |
| NC_045512.2_19mer_win1_28978 | 28978 | 28996 | 983 | TGGTAAAGGCCAACAACAA | 2186 | TTGTTGTTGGCCTTTACCA |
| NC_045512.2_19mer_win1_28979 | 28979 | 28997 | 984 | GGTAAAGGCCAACAACAAC | 2187 | GTTGTTGTTGGCCTTTACC |
| NC_045512.2_19mer_win1_28980 | 28980 | 28998 | 985 | GTAAAGGCCAACAACAACA | 2188 | TGTTGTTGTTGGCCTTTAC |
| NC_045512.2_19mer_win1_28981 | 28981 | 28999 | 986 | TAAAGGCCAACAACAACAA | 2189 | TTGTTGTTGTTGGCCTTTA |
| NC_045512.2_19mer_win1_28982 | 28982 | 29000 | 987 | AAAGGCCAACAACAACAAG | 2190 | CTTGTTGTTGTTGGCCTTT |
| NC_045512.2_19mer_win1_28983 | 28983 | 29001 | 988 | AAGGCCAACAACAACAAGG | 2191 | CCTTGTTGTTGTTGGCCTT |
| NC_045512.2_19mer_win1_28984 | 28984 | 29002 | 989 | AGGCCAACAACAACAAGGC | 2192 | GCCTTGTTGTTGTTGGCCT |
| NC_045512.2_19mer_win1_28985 | 28985 | 29003 | 990 | GGCCAACAACAACAAGGCC | 2193 | GGCCTTGTTGTTGTTGGCC |
| NC_045512.2_19mer_win1_28986 | 28986 | 29004 | 991 | GCCAACAACAACAAGGCCA | 2194 | TGGCCTTGTTGTTGTTGGC |
| NC_045512.2_19mer_win1_28987 | 28987 | 29005 | 992 | CCAACAACAACAAGGCCAA | 2195 | TTGGCCTTGTTGTTGTTGG |
| NC_045512.2_19mer_win1_28988 | 28988 | 29006 | 993 | CAACAACAACAAGGCCAAA | 2196 | TTTGGCCTTGTTGTTGTTG |
| NC_045512.2_19mer_win1_28989 | 28989 | 29007 | 994 | AACAACAACAAGGCCAAAC | 2197 | GTTTGGCCTTGTTGTTGTT |
| NC_045512.2_19mer_win1_28990 | 28990 | 29008 | 995 | ACAACAACAAGGCCAAACT | 2198 | AGTTTGGCCTTGTTGTTGT |
| NC_045512.2_19mer_win1_28991 | 28991 | 29009 | 996 | CAACAACAAGGCCAAACTG | 2199 | CAGTTTGGCCTTGTTGTTG |
| NC_045512.2_19mer_win1_28992 | 28992 | 29010 | 997 | AACAACAAGGCCAAACTGT | 2200 | ACAGTTTGGCCTTGTTGTT |
| NC_045512.2_19mer_win1_28993 | 28993 | 29011 | 998 | ACAACAAGGCCAAACTGTC | 2201 | GACAGTTTGGCCTTGTTGT |
| NC_045512.2_19mer_win1_28994 | 28994 | 29012 | 999 | CAACAAGGCCAAACTGTCA | 2202 | TGACAGTTTGGCCTTGTTG |
| NC_045512.2_19mer_win1_28995 | 28995 | 29013 | 1000 | AACAAGGCCAAACTGTCAC | 2203 | GTGACAGTTTGGCCTTGTT |
| NC_045512.2_19mer_win1_28996 | 28996 | 29014 | 1001 | ACAAGGCCAAACTGTCACT | 2204 | AGTGACAGTTTGGCCTTGT |
| NC_045512.2_19mer_win1_28997 | 28997 | 29015 | 1002 | CAAGGCCAAACTGTCACTA | 2205 | TAGTGACAGTTTGGCCTTG |
| NC_045512.2_19mer_win1_28998 | 28998 | 29016 | 1003 | AAGGCCAAACTGTCACTAA | 2206 | TTAGTGACAGTTTGGCCTT |
| NC_045512.2_19mer_win1_28999 | 28999 | 29017 | 1004 | AGGCCAAACTGTCACTAAG | 2207 | CTTAGTGACAGTTTGGCCT |
| NC_045512.2_19mer_win1_29000 | 29000 | 29018 | 1005 | GGCCAAACTGTCACTAAGA | 2208 | TCTTAGTGACAGTTTGGCC |
| NC_045512.2_19mer_win1_29001 | 29001 | 29019 | 1006 | GCCAAACTGTCACTAAGAA | 2209 | TTCTTAGTGACAGTTTGGC |
| NC_045512.2_19mer_win1_29002 | 29002 | 29020 | 1007 | CCAAACTGTCACTAAGAAA | 2210 | TTTCTTAGTGACAGTTTGG |
| NC_045512.2_19mer_win1_29003 | 29003 | 29021 | 1008 | CAAACTGTCACTAAGAAAT | 2211 | ATTTCTTAGTGACAGTTTG |
| NC_045512.2_19mer_win1_29004 | 29004 | 29022 | 1009 | AAACTGTCACTAAGAAATC | 2212 | GATTTCTTAGTGACAGTTT |
| NC_045512.2_19mer_win1_29005 | 29005 | 29023 | 1010 | AACTGTCACTAAGAAATCT | 2213 | AGATTTCTTAGTGACAGTT |
| NC_045512.2_19mer_win1_29006 | 29006 | 29024 | 1011 | ACTGTCACTAAGAAATCTG | 2214 | CAGATTTCTTAGTGACAGT |
| NC_045512.2_19mer_win1_29007 | 29007 | 29025 | 1012 | CTGTCACTAAGAAATCTGC | 2215 | GCAGATTTCTTAGTGACAG |
| NC_045512.2_19mer_win1_29008 | 29008 | 29026 | 1013 | TGTCACTAAGAAATCTGCT | 2216 | AGCAGATTTCTTAGTGACA |
| NC_045512.2_19mer_win1_29009 | 29009 | 29027 | 1014 | GTCACTAAGAAATCTGCTG | 2217 | CAGCAGATTTCTTAGTGAC |
| NC_045512.2_19mer_win1_29010 | 29010 | 29028 | 1015 | TCACTAAGAAATCTGCTGC | 2218 | GCAGCAGATTTCTTAGTGA |
| NC_045512.2_19mer_win1_29011 | 29011 | 29029 | 1016 | CACTAAGAAATCTGCTGCT | 2219 | AGCAGCAGATTTCTTAGTG |
| NC_045512.2_19mer_win1_29012 | 29012 | 29030 | 1017 | ACTAAGAAATCTGCTGCTG | 2220 | CAGCAGCAGATTTCTTAGT |
| NC_045512.2_19mer_win1_29013 | 29013 | 29031 | 1018 | CTAAGAAATCTGCTGCTGA | 2221 | TCAGCAGCAGATTTCTTAG |
| NC_045512.2_19mer_win1_29014 | 29014 | 29032 | 1019 | TAAGAAATCTGCTGCTGAG | 2222 | CTCAGCAGCAGATTTCTTA |
| NC_045512.2_19mer_win1_29015 | 29015 | 29033 | 1020 | AAGAAATCTGCTGCTGAGG | 2223 | CCTCAGCAGCAGATTTCTT |
| NC_045512.2_19mer_win1_29016 | 29016 | 29034 | 1021 | AGAAATCTGCTGCTGAGGC | 2224 | GCCTCAGCAGCAGATTTCT |
| NC_045512.2_19mer_win1_29144 | 29144 | 29162 | 1022 | CTAATCAGACAAGGAACTG | 2225 | CAGTTCCTTGTCTGATTAG |
| NC_045512.2_19mer_win1_29145 | 29145 | 29163 | 1023 | TAATCAGACAAGGAACTGA | 2226 | TCAGTTCCTTGTCTGATTA |
| NC_045512.2_19mer_win1_29146 | 29146 | 29164 | 1024 | AATCAGACAAGGAACTGAT | 2227 | ATCAGTTCCTTGTCTGATT |
| NC_045512.2_19mer_win1_29147 | 29147 | 29165 | 1025 | ATCAGACAAGGAACTGATT | 2228 | AATCAGTTCCTTGTCTGAT |
| NC_045512.2_19mer_win1_29148 | 29148 | 29166 | 1026 | TCAGACAAGGAACTGATTA | 2229 | TAATCAGTTCCTTGTCTGA |
| NC_045512.2_19mer_win1_29149 | 29149 | 29167 | 1027 | CAGACAAGGAACTGATTAC | 2230 | GTAATCAGTTCCTTGTCTG |
| NC_045512.2_19mer_win1_29150 | 29150 | 29168 | 1028 | AGACAAGGAACTGATTACA | 2231 | TGTAATCAGTTCCTTGTCT |
| NC_045512.2_19mer_win1_29151 | 29151 | 29169 | 1029 | GACAAGGAACTGATTACAA | 2232 | TTGTAATCAGTTCCTTGTC |
| NC_045512.2_19mer_win1_29152 | 29152 | 29170 | 1030 | ACAAGGAACTGATTACAAA | 2233 | TTTGTAATCAGTTCCTTGT |
| NC_045512.2_19mer_win1_29153 | 29153 | 29171 | 1031 | CAAGGAACTGATTACAAAC | 2234 | GTTTGTAATCAGTTCCTTG |
| NC_045512.2_19mer_win1_29154 | 29154 | 29172 | 1032 | AAGGAACTGATTACAAACA | 2235 | TGTTTGTAATCAGTTCCTT |
| NC_045512.2_19mer_win1_29174 | 29174 | 29192 | 1033 | TGGCCGCAAATTGCACAAT | 2236 | ATTGTGCAATTTGCGGCCA |
| NC_045512.2_19mer_win1_29175 | 29175 | 29193 | 1034 | GGCCGCAAATTGCACAATT | 2237 | AATTGTGCAATTTGCGGCC |
| NC_045512.2_19mer_win1_29176 | 29176 | 29194 | 1035 | GCCGCAAATTGCACAATTT | 2238 | AAATTGTGCAATTTGCGGC |
| NC_045512.2_19mer_win1_29177 | 29177 | 29195 | 1036 | CCGCAAATTGCACAATTTG | 2239 | CAAATTGTGCAATTTGCGG |
| NC_045512.2_19mer_win1_29178 | 29178 | 29196 | 1037 | CGCAAATTGCACAATTTGC | 2240 | GCAAATTGTGCAATTTGCG |
| NC_045512.2_19mer_win1_29228 | 29228 | 29246 | 1038 | CGCATTGGCATGGAAGTCA | 2241 | TGACTTCCATGCCAATGCG |
| NC_045512.2_19mer_win1_29229 | 29229 | 29247 | 1039 | GCATTGGCATGGAAGTCAC | 2242 | GTGACTTCCATGCCAATGC |
| NC_045512.2_19mer_win1_29230 | 29230 | 29248 | 1040 | CATTGGCATGGAAGTCACA | 2243 | TGTGACTTCCATGCCAATG |
| NC_045512.2_19mer_win1_29231 | 29231 | 29249 | 1041 | ATTGGCATGGAAGTCACAC | 2244 | GTGTGACTTCCATGCCAAT |
| NC_045512.2_19mer_win1_29232 | 29232 | 29250 | 1042 | TTGGCATGGAAGTCACACC | 2245 | GGTGTGACTTCCATGCCAA |
| NC_045512.2_19mer_win1_29233 | 29233 | 29251 | 1043 | TGGCATGGAAGTCACACCT | 2246 | AGGTGTGACTTCCATGCCA |
| NC_045512.2_19mer_win1_29234 | 29234 | 29252 | 1044 | GGCATGGAAGTCACACCTT | 2247 | AAGGTGTGACTTCCATGCC |
| NC_045512.2_19mer_win1_29235 | 29235 | 29253 | 1045 | GCATGGAAGTCACACCTTC | 2248 | GAAGGTGTGACTTCCATGC |
| NC_045512.2_19mer_win1_29236 | 29236 | 29254 | 1046 | CATGGAAGTCACACCTTCG | 2249 | CGAAGGTGTGACTTCCATG |
| NC_045512.2_19mer_win1_29237 | 29237 | 29255 | 1047 | ATGGAAGTCACACCTTCGG | 2250 | CCGAAGGTGTGACTTCCAT |
| NC_045512.2_19mer_win1_29238 | 29238 | 29256 | 1048 | TGGAAGTCACACCTTCGGG | 2251 | CCCGAAGGTGTGACTTCCA |
| NC_045512.2_19mer_win1_29239 | 29239 | 29257 | 1049 | GGAAGTCACACCTTCGGGA | 2252 | TCCCGAAGGTGTGACTTCC |
| NC_045512.2_19mer_win1_29240 | 29240 | 29258 | 1050 | GAAGTCACACCTTCGGGAA | 2253 | TTCCCGAAGGTGTGACTTC |
| NC_045512.2_19mer_win1_29241 | 29241 | 29259 | 1051 | AAGTCACACCTTCGGGAAC | 2254 | GTTCCCGAAGGTGTGACTT |
| NC_045512.2_19mer_win1_29285 | 29285 | 29303 | 1052 | AAATTGGATGACAAAGATC | 2255 | GATCTTTGTCATCCAATTT |
| NC_045512.2_19mer_win1_29286 | 29286 | 29304 | 1053 | AATTGGATGACAAAGATCC | 2256 | GGATCTTTGTCATCCAATT |
| NC_045512.2_19mer_win1_29287 | 29287 | 29305 | 1054 | ATTGGATGACAAAGATCCA | 2257 | TGGATCTTTGTCATCCAAT |
| NC_045512.2_19mer_win1_29342 | 29342 | 29360 | 1055 | ATTGACGCATACAAAACAT | 2258 | ATGTTTTGTATGCGTCAAT |
| NC_045512.2_19mer_win1_29343 | 29343 | 29361 | 1056 | TTGACGCATACAAAACATT | 2259 | AATGTTTTGTATGCGTCAA |
| NC_045512.2_19mer_win1_29344 | 29344 | 29362 | 1057 | TGACGCATACAAAACATTC | 2260 | GAATGTTTTGTATGCGTCA |
| NC_045512.2_19mer_win1_29345 | 29345 | 29363 | 1058 | GACGCATACAAAACATTCC | 2261 | GGAATGTTTTGTATGCGTC |
| NC_045512.2_19mer_win1_29346 | 29346 | 29364 | 1059 | ACGCATACAAAACATTCCC | 2262 | GGGAATGTTTTGTATGCGT |
| NC_045512.2_19mer_win1_29347 | 29347 | 29365 | 1060 | CGCATACAAAACATTCCCA | 2263 | TGGGAATGTTTTGTATGCG |
| NC_045512.2_19mer_win1_29348 | 29348 | 29366 | 1061 | GCATACAAAACATTCCCAC | 2264 | GTGGGAATGTTTTGTATGC |
| NC_045512.2_19mer_win1_29349 | 29349 | 29367 | 1062 | CATACAAAACATTCCCACC | 2265 | GGTGGGAATGTTTTGTATG |
| NC_045512.2_19mer_win1_29350 | 29350 | 29368 | 1063 | ATACAAAACATTCCCACCA | 2266 | TGGTGGGAATGTTTTGTAT |
| NC_045512.2_19mer_win1_29351 | 29351 | 29369 | 1064 | TACAAAACATTCCCACCAA | 2267 | TTGGTGGGAATGTTTTGTA |
| NC_045512.2_19mer_win1_29352 | 29352 | 29370 | 1065 | ACAAAACATTCCCACCAAC | 2268 | GTTGGTGGGAATGTTTTGT |
| NC_045512.2_19mer_win1_29353 | 29353 | 29371 | 1066 | CAAAACATTCCCACCAACA | 2269 | TGTTGGTGGGAATGTTTTG |
| NC_045512.2_19mer_win1_29354 | 29354 | 29372 | 1067 | AAAACATTCCCACCAACAG | 2270 | CTGTTGGTGGGAATGTTTT |
| NC_045512.2_19mer_win1_29355 | 29355 | 29373 | 1068 | AAACATTCCCACCAACAGA | 2271 | TCTGTTGGTGGGAATGTTT |
| NC_045512.2_19mer_win1_29356 | 29356 | 29374 | 1069 | AACATTCCCACCAACAGAG | 2272 | CTCTGTTGGTGGGAATGTT |
| NC_045512.2_19mer_win1_29357 | 29357 | 29375 | 1070 | ACATTCCCACCAACAGAGC | 2273 | GCTCTGTTGGTGGGAATGT |
| NC_045512.2_19mer_win1_29358 | 29358 | 29376 | 1071 | CATTCCCACCAACAGAGCC | 2274 | GGCTCTGTTGGTGGGAATG |
| NC_045512.2_19mer_win1_29359 | 29359 | 29377 | 1072 | ATTCCCACCAACAGAGCCT | 2275 | AGGCTCTGTTGGTGGGAAT |
| NC_045512.2_19mer_win1_29360 | 29360 | 29378 | 1073 | TTCCCACCAACAGAGCCTA | 2276 | TAGGCTCTGTTGGTGGGAA |
| NC_045512.2_19mer_win1_29361 | 29361 | 29379 | 1074 | TCCCACCAACAGAGCCTAA | 2277 | TTAGGCTCTGTTGGTGGGA |
| NC_045512.2_19mer_win1_29362 | 29362 | 29380 | 1075 | CCCACCAACAGAGCCTAAA | 2278 | TTTAGGCTCTGTTGGTGGG |
| NC_045512.2_19mer_win1_29363 | 29363 | 29381 | 1076 | CCACCAACAGAGCCTAAAA | 2279 | TTTTAGGCTCTGTTGGTGG |
| NC_045512.2_19mer_win1_29364 | 29364 | 29382 | 1077 | CACCAACAGAGCCTAAAAA | 2280 | TTTTTAGGCTCTGTTGGTG |
| NC_045512.2_19mer_win1_29365 | 29365 | 29383 | 1078 | ACCAACAGAGCCTAAAAAG | 2281 | CTTTTTAGGCTCTGTTGGT |
| NC_045512.2_19mer_win1_29366 | 29366 | 29384 | 1079 | CCAACAGAGCCTAAAAAGG | 2282 | CCTTTTTAGGCTCTGTTGG |
| NC_045512.2_19mer_win1_29367 | 29367 | 29385 | 1080 | CAACAGAGCCTAAAAAGGA | 2283 | TCCTTTTTAGGCTCTGTTG |
| NC_045512.2_19mer_win1_29368 | 29368 | 29386 | 1081 | AACAGAGCCTAAAAAGGAC | 2284 | GTCCTTTTTAGGCTCTGTT |
| NC_045512.2_19mer_win1_29369 | 29369 | 29387 | 1082 | ACAGAGCCTAAAAAGGACA | 2285 | TGTCCTTTTTAGGCTCTGT |
| NC_045512.2_19mer_win1_29370 | 29370 | 29388 | 1083 | CAGAGCCTAAAAAGGACAA | 2286 | TTGTCCTTTTTAGGCTCTG |
| NC_045512.2_19mer_win1_29371 | 29371 | 29389 | 1084 | AGAGCCTAAAAAGGACAAA | 2287 | TTTGTCCTTTTTAGGCTCT |
| NC_045512.2_19mer_win1_29372 | 29372 | 29390 | 1085 | GAGCCTAAAAAGGACAAAA | 2288 | TTTTGTCCTTTTTAGGCTC |
| NC_045512.2_19mer_win1_29373 | 29373 | 29391 | 1086 | AGCCTAAAAAGGACAAAAA | 2289 | TTTTTGTCCTTTTTAGGCT |
| NC_045512.2_19mer_win1_29374 | 29374 | 29392 | 1087 | GCCTAAAAAGGACAAAAAG | 2290 | CTTTTTGTCCTTTTTAGGC |
| NC_045512.2_19mer_win1_29375 | 29375 | 29393 | 1088 | CCTAAAAAGGACAAAAAGA | 2291 | TCTTTTTGTCCTTTTTAGG |
| NC_045512.2_19mer_win1_29376 | 29376 | 29394 | 1089 | CTAAAAAGGACAAAAAGAA | 2292 | TTCTTTTTGTCCTTTTTAG |
| NC_045512.2_19mer_win1_29444 | 29444 | 29462 | 1090 | ACTGTGACTCTTCTTCCTG | 2293 | CAGGAAGAAGAGTCACAGT |
| NC_045512.2_19mer_win1_29445 | 29445 | 29463 | 1091 | CTGTGACTCTTCTTCCTGC | 2294 | GCAGGAAGAAGAGTCACAG |
| NC_045512.2_19mer_win1_29543 | 29543 | 29561 | 1092 | GACCACACAAGGCAGATGG | 2295 | CCATCTGCCTTGTGTGGTC |
| NC_045512.2_19mer_win1_29544 | 29544 | 29562 | 1093 | ACCACACAAGGCAGATGGG | 2296 | CCCATCTGCCTTGTGTGGT |
| NC_045512.2_19mer_win1_29545 | 29545 | 29563 | 1094 | CCACACAAGGCAGATGGGC | 2297 | GCCCATCTGCCTTGTGTGG |
| NC_045512.2_19mer_win1_29546 | 29546 | 29564 | 1095 | CACACAAGGCAGATGGGCT | 2298 | AGCCCATCTGCCTTGTGTG |
| NC_045512.2_19mer_win1_29547 | 29547 | 29565 | 1096 | ACACAAGGCAGATGGGCTA | 2299 | TAGCCCATCTGCCTTGTGT |
| NC_045512.2_19mer_win1_29548 | 29548 | 29566 | 1097 | CACAAGGCAGATGGGCTAT | 2300 | ATAGCCCATCTGCCTTGTG |
| NC_045512.2_19mer_win1_29598 | 29598 | 29616 | 1098 | ATAGTCTACTCTTGTGCAG | 2301 | CTGCACAAGAGTAGACTAT |
| NC_045512.2_19mer_win1_29599 | 29599 | 29617 | 1099 | TAGTCTACTCTTGTGCAGA | 2302 | TCTGCACAAGAGTAGACTA |
| NC_045512.2_19mer_win1_29600 | 29600 | 29618 | 1100 | AGTCTACTCTTGTGCAGAA | 2303 | TTCTGCACAAGAGTAGACT |
| NC_045512.2_19mer_win1_29601 | 29601 | 29619 | 1101 | GTCTACTCTTGTGCAGAAT | 2304 | ATTCTGCACAAGAGTAGAC |
| NC_045512.2_19mer_win1_29602 | 29602 | 29620 | 1102 | TCTACTCTTGTGCAGAATG | 2305 | CATTCTGCACAAGAGTAGA |
| NC_045512.2_19mer_win1_29603 | 29603 | 29621 | 1103 | CTACTCTTGTGCAGAATGA | 2306 | TCATTCTGCACAAGAGTAG |
| NC_045512.2_19mer_win1_29604 | 29604 | 29622 | 1104 | TACTCTTGTGCAGAATGAA | 2307 | TTCATTCTGCACAAGAGTA |
| NC_045512.2_19mer_win1_29605 | 29605 | 29623 | 1105 | ACTCTTGTGCAGAATGAAT | 2308 | ATTCATTCTGCACAAGAGT |
| NC_045512.2_19mer_win1_29606 | 29606 | 29624 | 1106 | CTCTTGTGCAGAATGAATT | 2309 | AATTCATTCTGCACAAGAG |
| NC_045512.2_19mer_win1_29607 | 29607 | 29625 | 1107 | TCTTGTGCAGAATGAATTC | 2310 | GAATTCATTCTGCACAAGA |
| NC_045512.2_19mer_win1_29608 | 29608 | 29626 | 1108 | CTTGTGCAGAATGAATTCT | 2311 | AGAATTCATTCTGCACAAG |
| NC_045512.2_19mer_win1_29609 | 29609 | 29627 | 1109 | TTGTGCAGAATGAATTCTC | 2312 | GAGAATTCATTCTGCACAA |
| NC_045512.2_19mer_win1_29610 | 29610 | 29628 | 1110 | TGTGCAGAATGAATTCTCG | 2313 | CGAGAATTCATTCTGCACA |
| NC_045512.2_19mer_win1_29611 | 29611 | 29629 | 1111 | GTGCAGAATGAATTCTCGT | 2314 | ACGAGAATTCATTCTGCAC |
| NC_045512.2_19mer_win1_29612 | 29612 | 29630 | 1112 | TGCAGAATGAATTCTCGTA | 2315 | TACGAGAATTCATTCTGCA |
| NC_045512.2_19mer_win1_29652 | 29652 | 29670 | 1113 | TAGTTAACTTTAATCTCAC | 2316 | GTGAGATTAAAGTTAACTA |
| NC_045512.2_19mer_win1_29653 | 29653 | 29671 | 1114 | AGTTAACTTTAATCTCACA | 2317 | TGTGAGATTAAAGTTAACT |
| NC_045512.2_19mer_win1_29654 | 29654 | 29672 | 1115 | GTTAACTTTAATCTCACAT | 2318 | ATGTGAGATTAAAGTTAAC |
| NC_045512.2_19mer_win1_29655 | 29655 | 29673 | 1116 | TTAACTTTAATCTCACATA | 2319 | TATGTGAGATTAAAGTTAA |
| NC_045512.2_19mer_win1_29656 | 29656 | 29674 | 1117 | TAACTTTAATCTCACATAG | 2320 | CTATGTGAGATTAAAGTTA |
| NC_045512.2_19mer_win1_29657 | 29657 | 29675 | 1118 | AACTTTAATCTCACATAGC | 2321 | GCTATGTGAGATTAAAGTT |
| NC_045512.2_19mer_win1_29658 | 29658 | 29676 | 1119 | ACTTTAATCTCACATAGCA | 2322 | TGCTATGTGAGATTAAAGT |
| NC_045512.2_19mer_win1_29659 | 29659 | 29677 | 1120 | CTTTAATCTCACATAGCAA | 2323 | TTGCTATGTGAGATTAAAG |
| NC_045512.2_19mer_win1_29660 | 29660 | 29678 | 1121 | TTTAATCTCACATAGCAAT | 2324 | ATTGCTATGTGAGATTAAA |
| NC_045512.2_19mer_win1_29661 | 29661 | 29679 | 1122 | TTAATCTCACATAGCAATC | 2325 | GATTGCTATGTGAGATTAA |
| NC_045512.2_19mer_win1_29662 | 29662 | 29680 | 1123 | TAATCTCACATAGCAATCT | 2326 | AGATTGCTATGTGAGATTA |
| NC_045512.2_19mer_win1_29663 | 29663 | 29681 | 1124 | AATCTCACATAGCAATCTT | 2327 | AAGATTGCTATGTGAGATT |
| NC_045512.2_19mer_win1_29664 | 29664 | 29682 | 1125 | ATCTCACATAGCAATCTTT | 2328 | AAAGATTGCTATGTGAGAT |
| NC_045512.2_19mer_win1_29665 | 29665 | 29683 | 1126 | TCTCACATAGCAATCTTTA | 2329 | TAAAGATTGCTATGTGAGA |
| NC_045512.2_19mer_win1_29666 | 29666 | 29684 | 1127 | CTCACATAGCAATCTTTAA | 2330 | TTAAAGATTGCTATGTGAG |
| NC_045512.2_19mer_win1_29667 | 29667 | 29685 | 1128 | TCACATAGCAATCTTTAAT | 2331 | ATTAAAGATTGCTATGTGA |
| NC_045512.2_19mer_win1_29668 | 29668 | 29686 | 1129 | CACATAGCAATCTTTAATC | 2332 | GATTAAAGATTGCTATGTG |
| NC_045512.2_19mer_win1_29669 | 29669 | 29687 | 1130 | ACATAGCAATCTTTAATCA | 2333 | TGATTAAAGATTGCTATGT |
| NC_045512.2_19mer_win1_29689 | 29689 | 29707 | 1131 | TGTGTAACATTAGGGAGGA | 2334 | TCCTCCCTAATGTTACACA |
| NC_045512.2_19mer_win1_29690 | 29690 | 29708 | 1132 | GTGTAACATTAGGGAGGAC | 2335 | GTCCTCCCTAATGTTACAC |
| NC_045512.2_19mer_win1_29691 | 29691 | 29709 | 1133 | TGTAACATTAGGGAGGACT | 2336 | AGTCCTCCCTAATGTTACA |
| NC_045512.2_19mer_win1_29692 | 29692 | 29710 | 1134 | GTAACATTAGGGAGGACTT | 2337 | AAGTCCTCCCTAATGTTAC |
| NC_045512.2_19mer_win1_29693 | 29693 | 29711 | 1135 | TAACATTAGGGAGGACTTG | 2338 | CAAGTCCTCCCTAATGTTA |
| NC_045512.2_19mer_win1_29694 | 29694 | 29712 | 1136 | AACATTAGGGAGGACTTGA | 2339 | TCAAGTCCTCCCTAATGTT |
| NC_045512.2_19mer_win1_29695 | 29695 | 29713 | 1137 | ACATTAGGGAGGACTTGAA | 2340 | TTCAAGTCCTCCCTAATGT |
| NC_045512.2_19mer_win1_29696 | 29696 | 29714 | 1138 | CATTAGGGAGGACTTGAAA | 2341 | TTTCAAGTCCTCCCTAATG |
| NC_045512.2_19mer_win1_29697 | 29697 | 29715 | 1139 | ATTAGGGAGGACTTGAAAG | 2342 | CTTTCAAGTCCTCCCTAAT |
| NC_045512.2_19mer_win1_29698 | 29698 | 29716 | 1140 | TTAGGGAGGACTTGAAAGA | 2343 | TCTTTCAAGTCCTCCCTAA |
| NC_045512.2_19mer_win1_29699 | 29699 | 29717 | 1141 | TAGGGAGGACTTGAAAGAG | 2344 | CTCTTTCAAGTCCTCCCTA |
| NC_045512.2_19mer_win1_29700 | 29700 | 29718 | 1142 | AGGGAGGACTTGAAAGAGC | 2345 | GCTCTTTCAAGTCCTCCCT |
| NC_045512.2_19mer_win1_29701 | 29701 | 29719 | 1143 | GGGAGGACTTGAAAGAGCC | 2346 | GGCTCTTTCAAGTCCTCCC |
| NC_045512.2_19mer_win1_29702 | 29702 | 29720 | 1144 | GGAGGACTTGAAAGAGCCA | 2347 | TGGCTCTTTCAAGTCCTCC |
| NC_045512.2_19mer_win1_29703 | 29703 | 29721 | 1145 | GAGGACTTGAAAGAGCCAC | 2348 | GTGGCTCTTTCAAGTCCTC |
| NC_045512.2_19mer_win1_29704 | 29704 | 29722 | 1146 | AGGACTTGAAAGAGCCACC | 2349 | GGTGGCTCTTTCAAGTCCT |
| NC_045512.2_19mer_win1_29705 | 29705 | 29723 | 1147 | GGACTTGAAAGAGCCACCA | 2350 | TGGTGGCTCTTTCAAGTCC |
| NC_045512.2_19mer_win1_29706 | 29706 | 29724 | 1148 | GACTTGAAAGAGCCACCAC | 2351 | GTGGTGGCTCTTTCAAGTC |
| NC_045512.2_19mer_win1_29707 | 29707 | 29725 | 1149 | ACTTGAAAGAGCCACCACA | 2352 | TGTGGTGGCTCTTTCAAGT |
| NC_045512.2_19mer_win1_29708 | 29708 | 29726 | 1150 | CTTGAAAGAGCCACCACAT | 2353 | ATGTGGTGGCTCTTTCAAG |
| NC_045512.2_19mer_win1_29709 | 29709 | 29727 | 1151 | TTGAAAGAGCCACCACATT | 2354 | AATGTGGTGGCTCTTTCAA |
| NC_045512.2_19mer_win1_29710 | 29710 | 29728 | 1152 | TGAAAGAGCCACCACATTT | 2355 | AAATGTGGTGGCTCTTTCA |
| NC_045512.2_19mer_win1_29711 | 29711 | 29729 | 1153 | GAAAGAGCCACCACATTTT | 2356 | AAAATGTGGTGGCTCTTTC |
| NC_045512.2_19mer_win1_29712 | 29712 | 29730 | 1154 | AAAGAGCCACCACATTTTC | 2357 | GAAAATGTGGTGGCTCTTT |
| NC_045512.2_19mer_win1_29713 | 29713 | 29731 | 1155 | AAGAGCCACCACATTTTCA | 2358 | TGAAAATGTGGTGGCTCTT |
| NC_045512.2_19mer_win1_29733 | 29733 | 29751 | 1156 | CGAGGCCACGCGGAGTACG | 2359 | CGTACTCCGCGTGGCCTCG |
| NC_045512.2_19mer_win1_29734 | 29734 | 29752 | 1157 | GAGGCCACGCGGAGTACGA | 2360 | TCGTACTCCGCGTGGCCTC |
| NC_045512.2_19mer_win1_29735 | 29735 | 29753 | 1158 | AGGCCACGCGGAGTACGAT | 2361 | ATCGTACTCCGCGTGGCCT |
| NC_045512.2_19mer_win1_29736 | 29736 | 29754 | 1159 | GGCCACGCGGAGTACGATC | 2362 | GATCGTACTCCGCGTGGCC |
| NC_045512.2_19mer_win1_29737 | 29737 | 29755 | 1160 | GCCACGCGGAGTACGATCG | 2363 | CGATCGTACTCCGCGTGGC |
| NC_045512.2_19mer_win1_29738 | 29738 | 29756 | 1161 | CCACGCGGAGTACGATCGA | 2364 | TCGATCGTACTCCGCGTGG |
| NC_045512.2_19mer_win1_29739 | 29739 | 29757 | 1162 | CACGCGGAGTACGATCGAG | 2365 | CTCGATCGTACTCCGCGTG |
| NC_045512.2_19mer_win1_29770 | 29770 | 29788 | 1163 | AATGCTAGGGAGAGCTGCC | 2366 | GGCAGCTCTCCCTAGCATT |
| NC_045512.2_19mer_win1_29771 | 29771 | 29789 | 1164 | ATGCTAGGGAGAGCTGCCT | 2367 | AGGCAGCTCTCCCTAGCAT |
| NC_045512.2_19mer_win1_29772 | 29772 | 29790 | 1165 | TGCTAGGGAGAGCTGCCTA | 2368 | TAGGCAGCTCTCCCTAGCA |
| NC_045512.2_19mer_win1_29773 | 29773 | 29791 | 1166 | GCTAGGGAGAGCTGCCTAT | 2369 | ATAGGCAGCTCTCCCTAGC |
| NC_045512.2_19mer_win1_29774 | 29774 | 29792 | 1167 | CTAGGGAGAGCTGCCTATA | 2370 | TATAGGCAGCTCTCCCTAG |
| NC_045512.2_19mer_win1_29775 | 29775 | 29793 | 1168 | TAGGGAGAGCTGCCTATAT | 2371 | ATATAGGCAGCTCTCCCTA |
| NC_045512.2_19mer_win1_29776 | 29776 | 29794 | 1169 | AGGGAGAGCTGCCTATATG | 2372 | CATATAGGCAGCTCTCCCT |
| NC_045512.2_19mer_win1_29777 | 29777 | 29795 | 1170 | GGGAGAGCTGCCTATATGG | 2373 | CCATATAGGCAGCTCTCCC |
| NC_045512.2_19mer_win1_29778 | 29778 | 29796 | 1171 | GGAGAGCTGCCTATATGGA | 2374 | TCCATATAGGCAGCTCTCC |
| NC_045512.2_19mer_win1_29779 | 29779 | 29797 | 1172 | GAGAGCTGCCTATATGGAA | 2375 | TTCCATATAGGCAGCTCTC |
| NC_045512.2_19mer_win1_29780 | 29780 | 29798 | 1173 | AGAGCTGCCTATATGGAAG | 2376 | CTTCCATATAGGCAGCTCT |
| NC_045512.2_19mer_win1_29781 | 29781 | 29799 | 1174 | GAGCTGCCTATATGGAAGA | 2377 | TCTTCCATATAGGCAGCTC |
| NC_045512.2_19mer_win1_29782 | 29782 | 29800 | 1175 | AGCTGCCTATATGGAAGAG | 2378 | CTCTTCCATATAGGCAGCT |
| NC_045512.2_19mer_win1_29783 | 29783 | 29801 | 1176 | GCTGCCTATATGGAAGAGC | 2379 | GCTCTTCCATATAGGCAGC |
| NC_045512.2_19mer_win1_29784 | 29784 | 29802 | 1177 | CTGCCTATATGGAAGAGCC | 2380 | GGCTCTTCCATATAGGCAG |
| NC_045512.2_19mer_win1_29785 | 29785 | 29803 | 1178 | TGCCTATATGGAAGAGCCC | 2381 | GGGCTCTTCCATATAGGCA |
| NC_045512.2_19mer_win1_29786 | 29786 | 29804 | 1179 | GCCTATATGGAAGAGCCCT | 2382 | AGGGCTCTTCCATATAGGC |
| NC_045512.2_19mer_win1_29787 | 29787 | 29805 | 1180 | CCTATATGGAAGAGCCCTA | 2383 | TAGGGCTCTTCCATATAGG |
| NC_045512.2_19mer_win1_29788 | 29788 | 29806 | 1181 | CTATATGGAAGAGCCCTAA | 2384 | TTAGGGCTCTTCCATATAG |
| NC_045512.2_19mer_win1_29789 | 29789 | 29807 | 1182 | TATATGGAAGAGCCCTAAT | 2385 | ATTAGGGCTCTTCCATATA |
| NC_045512.2_19mer_win1_29790 | 29790 | 29808 | 1183 | ATATGGAAGAGCCCTAATG | 2386 | CATTAGGGCTCTTCCATAT |
| NC_045512.2_19mer_win1_29791 | 29791 | 29809 | 1184 | TATGGAAGAGCCCTAATGT | 2387 | ACATTAGGGCTCTTCCATA |
| NC_045512.2_19mer_win1_29792 | 29792 | 29810 | 1185 | ATGGAAGAGCCCTAATGTG | 2388 | CACATTAGGGCTCTTCCAT |
| NC_045512.2_19mer_win1_29793 | 29793 | 29811 | 1186 | TGGAAGAGCCCTAATGTGT | 2389 | ACACATTAGGGCTCTTCCA |
| NC_045512.2_19mer_win1_29794 | 29794 | 29812 | 1187 | GGAAGAGCCCTAATGTGTA | 2390 | TACACATTAGGGCTCTTCC |
| NC_045512.2_19mer_win1_29795 | 29795 | 29813 | 1188 | GAAGAGCCCTAATGTGTAA | 2391 | TTACACATTAGGGCTCTTC |
| NC_045512.2_19mer_win1_29796 | 29796 | 29814 | 1189 | AAGAGCCCTAATGTGTAAA | 2392 | TTTACACATTAGGGCTCTT |
| NC_045512.2_19mer_win1_29797 | 29797 | 29815 | 1190 | AGAGCCCTAATGTGTAAAA | 2393 | TTTTACACATTAGGGCTCT |
| NC_045512.2_19mer_win1_29798 | 29798 | 29816 | 1191 | GAGCCCTAATGTGTAAAAT | 2394 | ATTTTACACATTAGGGCTC |
| NC_045512.2_19mer_win1_29799 | 29799 | 29817 | 1192 | AGCCCTAATGTGTAAAATT | 2395 | AATTTTACACATTAGGGCT |
| NC_045512.2_19mer_win1_29800 | 29800 | 29818 | 1193 | GCCCTAATGTGTAAAATTA | 2396 | TAATTTTACACATTAGGGC |
| NC_045512.2_19mer_win1_29801 | 29801 | 29819 | 1194 | CCCTAATGTGTAAAATTAA | 2397 | TTAATTTTACACATTAGGG |
| NC_045512.2_19mer_win1_29802 | 29802 | 29820 | 1195 | CCTAATGTGTAAAATTAAT | 2398 | ATTAATTTTACACATTAGG |
| NC_045512.2_19mer_win1_29803 | 29803 | 29821 | 1196 | CTAATGTGTAAAATTAATT | 2399 | AATTAATTTTACACATTAG |
| NC_045512.2_19mer_win1_29804 | 29804 | 29822 | 1197 | TAATGTGTAAAATTAATTT | 2400 | AAATTAATTTTACACATTA |
| NC_045512.2_19mer_win1_29805 | 29805 | 29823 | 1198 | AATGTGTAAAATTAATTTT | 2401 | AAAATTAATTTTACACATT |
| NC_045512.2_19mer_win1_29806 | 29806 | 29824 | 1199 | ATGTGTAAAATTAATTTTA | 2402 | TAAAATTAATTTTACACAT |
| NC_045512.2_19mer_win1_29807 | 29807 | 29825 | 1200 | TGTGTAAAATTAATTTTAG | 2403 | CTAAAATTAATTTTACACA |
| NC_045512.2_19mer_win1_29808 | 29808 | 29826 | 1201 | GTGTAAAATTAATTTTAGT | 2404 | ACTAAAATTAATTTTACAC |
| NC_045512.2_19mer_win1_29809 | 29809 | 29827 | 1202 | TGTAAAATTAATTTTAGTA | 2405 | TACTAAAATTAATTTTACA |
| NC_045512.2_19mer_win1_29810 | 29810 | 29828 | 1203 | GTAAAATTAATTTTAGTAG | 2406 | CTACTAAAATTAATTTTAC |
| 21-mer Target Sequences | | | | | | |
| NC_045512.2_21mer_win1_00190 | 190 | 210 | 2411 | GACGAATGCCAAAGCAGGCAC | 3393 | CACGGACGAAACCGTAAGCAG |
| NC_045512.2_21mer_win1_00191 | 191 | 211 | 2412 | ACGAATGCCAAAGCAGGCACA | 3394 | ACACGGACGAAACCGTAAGCA |
| NC_045512.2_21mer_win1_00192 | 192 | 212 | 2413 | CGAATGCCAAAGCAGGCACAA | 3395 | AACACGGACGAAACCGTAAGC |
| NC_045512.2_21mer_win1_00193 | 193 | 213 | 2414 | GAATGCCAAAGCAGGCACAAC | 3396 | CAACACGGACGAAACCGTAAG |
| NC_045512.2_21mer_win1_00194 | 194 | 214 | 2415 | AATGCCAAAGCAGGCACAACG | 3397 | GCAACACGGACGAAACCGTAA |
| NC_045512.2_21mer_win1_00195 | 195 | 215 | 2416 | ATGCCAAAGCAGGCACAACGT | 3398 | TGCAACACGGACGAAACCGTA |
| NC_045512.2_21mer_win1_00196 | 196 | 216 | 2417 | TGCCAAAGCAGGCACAACGTC | 3399 | CTGCAACACGGACGAAACCGT |
| NC_045512.2_21mer_win1_00233 | 233 | 253 | 2418 | GATCCAAAGCAGGCCCACACT | 3400 | TCACACCCGGACGAAACCTAG |
| NC_045512.2_21mer_win1_00234 | 234 | 254 | 2419 | ATCCAAAGCAGGCCCACACTG | 3401 | GTCACACCCGGACGAAACCTA |
| NC_045512.2_21mer_win1_00235 | 235 | 255 | 2420 | TCCAAAGCAGGCCCACACTGG | 3402 | GGTCACACCCGGACGAAACCT |
| NC_045512.2_21mer_win1_00236 | 236 | 256 | 2421 | CCAAAGCAGGCCCACACTGGC | 3403 | CGGTCACACCCGGACGAAACC |
| NC_045512.2_21mer_win1_00237 | 237 | 257 | 2422 | CAAAGCAGGCCCACACTGGCT | 3404 | TCGGTCACACCCGGACGAAAC |
| NC_045512.2_21mer_win1_00238 | 238 | 258 | 2423 | AAAGCAGGCCCACACTGGCTT | 3405 | TTCGGTCACACCCGGACGAAA |
| NC_045512.2_21mer_win1_00239 | 239 | 259 | 2424 | AAGCAGGCCCACACTGGCTTT | 3406 | TTTCGGTCACACCCGGACGAA |
| NC_045512.2_21mer_win1_00240 | 240 | 260 | 2425 | AGCAGGCCCACACTGGCTTTC | 3407 | CTTTCGGTCACACCCGGACGA |
| NC_045512.2_21mer_win1_00241 | 241 | 261 | 2426 | GCAGGCCCACACTGGCTTTCC | 3408 | CCTTTCGGTCACACCCGGACG |
| NC_045512.2_21mer_win1_00242 | 242 | 262 | 2427 | CAGGCCCACACTGGCTTTCCA | 3409 | ACCTTTCGGTCACACCCGGAC |
| NC_045512.2_21mer_win1_00243 | 243 | 263 | 2428 | AGGCCCACACTGGCTTTCCAT | 3410 | TACCTTTCGGTCACACCCGGA |
| NC_045512.2_21mer_win1_00244 | 244 | 264 | 2429 | GGCCCACACTGGCTTTCCATT | 3411 | TTACCTTTCGGTCACACCCGG |
| NC_045512.2_21mer_win1_00245 | 245 | 265 | 2430 | GCCCACACTGGCTTTCCATTC | 3412 | CTTACCTTTCGGTCACACCCG |
| NC_045512.2_21mer_win1_00246 | 246 | 266 | 2431 | CCCACACTGGCTTTCCATTCT | 3413 | TCTTACCTTTCGGTCACACCC |
| NC_045512.2_21mer_win1_00247 | 247 | 267 | 2432 | CCACACTGGCTTTCCATTCTA | 3414 | ATCTTACCTTTCGGTCACACC |
| NC_045512.2_21mer_win1_00248 | 248 | 268 | 2433 | CACACTGGCTTTCCATTCTAC | 3415 | CATCTTACCTTTCGGTCACAC |
| NC_045512.2_21mer_win1_00249 | 249 | 269 | 2434 | ACACTGGCTTTCCATTCTACC | 3416 | CCATCTTACCTTTCGGTCACA |
| NC_045512.2_21mer_win1_00250 | 250 | 270 | 2435 | CACTGGCTTTCCATTCTACCT | 3417 | TCCATCTTACCTTTCGGTCAC |
| NC_045512.2_21mer_win1_00251 | 251 | 271 | 2436 | ACTGGCTTTCCATTCTACCTC | 3418 | CTCCATCTTACCTTTCGGTCA |
| NC_045512.2_21mer_win1_00252 | 252 | 272 | 2437 | CTGGCTTTCCATTCTACCTCT | 3419 | TCTCCATCTTACCTTTCGGTC |
| NC_045512.2_21mer_win1_00253 | 253 | 273 | 2438 | TGGCTTTCCATTCTACCTCTC | 3420 | CTCTCCATCTTACCTTTCGGT |
| NC_045512.2_21mer_win1_00254 | 254 | 274 | 2439 | GGCTTTCCATTCTACCTCTCG | 3421 | GCTCTCCATCTTACCTTTCGG |
| NC_045512.2_21mer_win1_00255 | 255 | 275 | 2440 | GCTTTCCATTCTACCTCTCGG | 3422 | GGCTCTCCATCTTACCTTTCG |
| NC_045512.2_21mer_win1_00256 | 256 | 276 | 2441 | CTTTCCATTCTACCTCTCGGA | 3423 | AGGCTCTCCATCTTACCTTTC |
| NC_045512.2_21mer_win1_00257 | 257 | 277 | 2442 | TTTCCATTCTACCTCTCGGAA | 3424 | AAGGCTCTCCATCTTACCTTT |
| NC_045512.2_21mer_win1_00258 | 258 | 278 | 2443 | TTCCATTCTACCTCTCGGAAC | 3425 | CAAGGCTCTCCATCTTACCTT |
| NC_045512.2_21mer_win1_00259 | 259 | 279 | 2444 | TCCATTCTACCTCTCGGAACA | 3426 | ACAAGGCTCTCCATCTTACCT |
| NC_045512.2_21mer_win1_00288 | 288 | 308 | 2445 | AGTTGCTCTTTTGTGTGCAGG | 3427 | GGACGTGTGTTTTCTCGTTGA |
| NC_045512.2_21mer_win1_00289 | 289 | 309 | 2446 | GTTGCTCTTTTGTGTGCAGGT | 3428 | TGGACGTGTGTTTTCTCGTTG |
| NC_045512.2_21mer_win1_00290 | 290 | 310 | 2447 | TTGCTCTTTTGTGTGCAGGTT | 3429 | TTGGACGTGTGTTTTCTCGTT |
| NC_045512.2_21mer_win1_00291 | 291 | 311 | 2448 | TGCTCTTTTGTGTGCAGGTTG | 3430 | GTTGGACGTGTGTTTTCTCGT |
| NC_045512.2_21mer_win1_00292 | 292 | 312 | 2449 | GCTCTTTTGTGTGCAGGTTGA | 3431 | AGTTGGACGTGTGTTTTCTCG |
| NC_045512.2_21mer_win1_00293 | 293 | 313 | 2450 | CTCTTTTGTGTGCAGGTTGAG | 3432 | GAGTTGGACGTGTGTTTTCTC |
| NC_045512.2_21mer_win1_00294 | 294 | 314 | 2451 | TCTTTTGTGTGCAGGTTGAGT | 3433 | TGAGTTGGACGTGTGTTTTCT |
| NC_045512.2_21mer_win1_00295 | 295 | 315 | 2452 | CTTTTGTGTGCAGGTTGAGTC | 3434 | CTGAGTTGGACGTGTGTTTTC |
| NC_045512.2_21mer_win1_00296 | 296 | 316 | 2453 | TTTTGTGTGCAGGTTGAGTCA | 3435 | ACTGAGTTGGACGTGTGTTTT |
| NC_045512.2_21mer_win1_00297 | 297 | 317 | 2454 | TTTGTGTGCAGGTTGAGTCAA | 3436 | AACTGAGTTGGACGTGTGTTT |
| NC_045512.2_21mer_win1_00298 | 298 | 318 | 2455 | TTGTGTGCAGGTTGAGTCAAA | 3437 | AAACTGAGTTGGACGTGTGTT |
| NC_045512.2_21mer_win1_00299 | 299 | 319 | 2456 | TGTGTGCAGGTTGAGTCAAAC | 3438 | CAAACTGAGTTGGACGTGTGT |
| NC_045512.2_21mer_win1_00300 | 300 | 320 | 2457 | GTGTGCAGGTTGAGTCAAACG | 3439 | GCAAACTGAGTTGGACGTGTG |
| NC_045512.2_21mer_win1_00301 | 301 | 321 | 2458 | TGTGCAGGTTGAGTCAAACGG | 3440 | GGCAAACTGAGTTGGACGTGT |
| NC_045512.2_21mer_win1_00302 | 302 | 322 | 2459 | GTGCAGGTTGAGTCAAACGGA | 3441 | AGGCAAACTGAGTTGGACGTG |
| NC_045512.2_21mer_win1_00303 | 303 | 323 | 2460 | TGCAGGTTGAGTCAAACGGAC | 3442 | CAGGCAAACTGAGTTGGACGT |
| NC_045512.2_21mer_win1_00304 | 304 | 324 | 2461 | GCAGGTTGAGTCAAACGGACA | 3443 | ACAGGCAAACTGAGTTGGACG |
| NC_045512.2_21mer_win1_00455 | 455 | 475 | 2462 | GAACTTGTCGGGATACACAAG | 3444 | GAACACATAGGGCTGTTCAAG |
| NC_045512.2_21mer_win1_00456 | 456 | 476 | 2463 | AACTTGTCGGGATACACAAGT | 3445 | TGAACACATAGGGCTGTTCAA |
| NC_045512.2_21mer_win1_00457 | 457 | 477 | 2464 | ACTTGTCGGGATACACAAGTA | 3446 | ATGAACACATAGGGCTGTTCA |
| NC_045512.2_21mer_win1_00626 | 626 | 646 | 2465 | CAAGAAGAAGCATTCTTGCCA | 3447 | ACCGTTCTTACGAAGAAGAAC |
| NC_045512.2_21mer_win1_00627 | 627 | 647 | 2466 | AAGAAGAAGCATTCTTGCCAT | 3448 | TACCGTTCTTACGAAGAAGAA |
| NC_045512.2_21mer_win1_00628 | 628 | 648 | 2467 | AGAAGAAGCATTCTTGCCATT | 3449 | TTACCGTTCTTACGAAGAAGA |
| NC_045512.2_21mer_win1_00629 | 629 | 649 | 2468 | GAAGAAGCATTCTTGCCATTA | 3450 | ATTACCGTTCTTACGAAGAAG |
| NC_045512.2_21mer_win1_00630 | 630 | 650 | 2469 | AAGAAGCATTCTTGCCATTAT | 3451 | TATTACCGTTCTTACGAAGAA |
| NC_045512.2_21mer_win1_00631 | 631 | 651 | 2470 | AGAAGCATTCTTGCCATTATT | 3452 | TTATTACCGTTCTTACGAAGA |
| NC_045512.2_21mer_win1_03352 | 3352 | 3372 | 2471 | ACCAATAAATTTTGAATGACT | 3453 | TCAGTAAGTTTTAAATAACCA |
| NC_045512.2_21mer_win1_03353 | 3353 | 3373 | 2472 | CCAATAAATTTTGAATGACTG | 3454 | GTCAGTAAGTTTTAAATAACC |
| NC_045512.2_21mer_win1_03354 | 3354 | 3374 | 2473 | CAATAAATTTTGAATGACTGT | 3455 | TGTCAGTAAGTTTTAAATAAC |
| NC_045512.2_21mer_win1_03355 | 3355 | 3375 | 2474 | AATAAATTTTGAATGACTGTT | 3456 | TTGTCAGTAAGTTTTAAATAA |
| NC_045512.2_21mer_win1_03356 | 3356 | 3376 | 2475 | ATAAATTTTGAATGACTGTTA | 3457 | ATTGTCAGTAAGTTTTAAATA |
| NC_045512.2_21mer_win1_03357 | 3357 | 3377 | 2476 | TAAATTTTGAATGACTGTTAC | 3458 | CATTGTCAGTAAGTTTTAAAT |
| NC_045512.2_21mer_win1_03358 | 3358 | 3378 | 2477 | AAATTTTGAATGACTGTTACA | 3459 | ACATTGTCAGTAAGTTTTAAA |
| NC_045512.2_21mer_win1_06406 | 6406 | 6426 | 2478 | GAGACTTCTTCATCACCTTTT | 3460 | TTTTCCACTACTTCTTCAGAG |
| NC_045512.2_21mer_win1_06407 | 6407 | 6427 | 2479 | AGACTTCTTCATCACCTTTTA | 3461 | ATTTTCCACTACTTCTTCAGA |
| NC_045512.2_21mer_win1_06408 | 6408 | 6428 | 2480 | GACTTCTTCATCACCTTTTAG | 3462 | GATTTTCCACTACTTCTTCAG |
| NC_045512.2_21mer_win1_06409 | 6409 | 6429 | 2481 | ACTTCTTCATCACCTTTTAGG | 3463 | GGATTTTCCACTACTTCTTCA |
| NC_045512.2_21mer_win1_06410 | 6410 | 6430 | 2482 | CTTCTTCATCACCTTTTAGGA | 3464 | AGGATTTTCCACTACTTCTTC |
| NC_045512.2_21mer_win1_06411 | 6411 | 6431 | 2483 | TTCTTCATCACCTTTTAGGAT | 3465 | TAGGATTTTCCACTACTTCTT |
| NC_045512.2_21mer_win1_06412 | 6412 | 6432 | 2484 | TCTTCATCACCTTTTAGGATG | 3466 | GTAGGATTTTCCACTACTTCT |
| NC_045512.2_21mer_win1_06413 | 6413 | 6433 | 2485 | CTTCATCACCTTTTAGGATGG | 3467 | GGTAGGATTTTCCACTACTTC |
| NC_045512.2_21mer_win1_06461 | 6461 | 6481 | 2486 | CACTTTTGATGGCTTCAACAT | 3468 | TACAACTTCGGTAGTTTTCAC |
| NC_045512.2_21mer_win1_06462 | 6462 | 6482 | 2487 | ACTTTTGATGGCTTCAACATC | 3469 | CTACAACTTCGGTAGTTTTCA |
| NC_045512.2_21mer_win1_06463 | 6463 | 6483 | 2488 | CTTTTGATGGCTTCAACATCC | 3470 | CCTACAACTTCGGTAGTTTTC |
| NC_045512.2_21mer_win1_10484 | 10484 | 10504 | 2489 | AGTACACCATCACAACCAAAA | 3471 | AAAACCAACACTACCACATGA |
| NC_045512.2_21mer_win1_10485 | 10485 | 10505 | 2490 | GTACACCATCACAACCAAAAT | 3472 | TAAAACCAACACTACCACATG |
| NC_045512.2_21mer_win1_10486 | 10486 | 10506 | 2491 | TACACCATCACAACCAAAATT | 3473 | TTAAAACCAACACTACCACAT |
| NC_045512.2_21mer_win1_10487 | 10487 | 10507 | 2492 | ACACCATCACAACCAAAATTG | 3474 | GTTAAAACCAACACTACCACA |
| NC_045512.2_21mer_win1_10488 | 10488 | 10508 | 2493 | CACCATCACAACCAAAATTGT | 3475 | TGTTAAAACCAACACTACCAC |
| NC_045512.2_21mer_win1_10489 | 10489 | 10509 | 2494 | ACCATCACAACCAAAATTGTA | 3476 | ATGTTAAAACCAACACTACCA |
| NC_045512.2_21mer_win1_11609 | 11609 | 11629 | 2495 | CAAATAACAAAGAATCCGATA | 3477 | ATAGCCTAAGAAACAATAAAC |
| NC_045512.2_21mer_win1_11610 | 11610 | 11630 | 2496 | AAATAACAAAGAATCCGATAA | 3478 | AATAGCCTAAGAAACAATAAA |
| NC_045512.2_21mer_win1_12023 | 12023 | 12043 | 2497 | AGGTACGTCCCACGACATCTG | 3479 | GTCTACAGCACCCTGCATGGA |
| NC_045512.2_21mer_win1_12024 | 12024 | 12044 | 2498 | GGTACGTCCCACGACATCTGT | 3480 | TGTCTACAGCACCCTGCATGG |
| NC_045512.2_21mer_win1_12025 | 12025 | 12045 | 2499 | GTACGTCCCACGACATCTGTA | 3481 | ATGTCTACAGCACCCTGCATG |
| NC_045512.2_21mer_win1_12212 | 12212 | 12232 | 2500 | AGAAACTTACACCGATTTAGA | 3482 | AGATTTAGCCACATTCAAAGA |
| NC_045512.2_21mer_win1_12213 | 12213 | 12233 | 2501 | GAAACTTACACCGATTTAGAC | 3483 | CAGATTTAGCCACATTCAAAG |
| NC_045512.2_21mer_win1_12214 | 12214 | 12234 | 2502 | AAACTTACACCGATTTAGACT | 3484 | TCAGATTTAGCCACATTCAAA |
| NC_045512.2_21mer_win1_12839 | 12839 | 12859 | 2503 | TTTACCCGATCTAAGGGATTC | 3485 | CTTAGGGAATCTAGCCCATTT |
| NC_045512.2_21mer_win1_12840 | 12840 | 12860 | 2504 | TTACCCGATCTAAGGGATTCT | 3486 | TCTTAGGGAATCTAGCCCATT |
| NC_045512.2_21mer_win1_12841 | 12841 | 12861 | 2505 | TACCCGATCTAAGGGATTCTC | 3487 | CTCTTAGGGAATCTAGCCCAT |
| NC_045512.2_21mer_win1_12842 | 12842 | 12862 | 2506 | ACCCGATCTAAGGGATTCTCA | 3488 | ACTCTTAGGGAATCTAGCCCA |
| NC_045512.2_21mer_win1_12843 | 12843 | 12863 | 2507 | CCCGATCTAAGGGATTCTCAC | 3489 | CACTCTTAGGGAATCTAGCCC |
| NC_045512.2_21mer_win1_12844 | 12844 | 12864 | 2508 | CCGATCTAAGGGATTCTCACT | 3490 | TCACTCTTAGGGAATCTAGCC |
| NC_045512.2_21mer_win1_12845 | 12845 | 12865 | 2509 | CGATCTAAGGGATTCTCACTA | 3491 | ATCACTCTTAGGGAATCTAGC |
| NC_045512.2_21mer_win1_12846 | 12846 | 12866 | 2510 | GATCTAAGGGATTCTCACTAC | 3492 | CATCACTCTTAGGGAATCTAG |
| NC_045512.2_21mer_win1_12847 | 12847 | 12867 | 2511 | ATCTAAGGGATTCTCACTACC | 3493 | CCATCACTCTTAGGGAATCTA |
| NC_045512.2_21mer_win1_12885 | 12885 | 12905 | 2512 | GTCTTGACCTTGGTGGAACAT | 3494 | TACAAGGTGGTTCCAGTTCTG |
| NC_045512.2_21mer_win1_12886 | 12886 | 12906 | 2513 | TCTTGACCTTGGTGGAACATC | 3495 | CTACAAGGTGGTTCCAGTTCT |
| NC_045512.2_21mer_win1_12887 | 12887 | 12907 | 2514 | CTTGACCTTGGTGGAACATCC | 3496 | CCTACAAGGTGGTTCCAGTTC |
| NC_045512.2_21mer_win1_12888 | 12888 | 12908 | 2515 | TTGACCTTGGTGGAACATCCA | 3497 | ACCTACAAGGTGGTTCCAGTT |
| NC_045512.2_21mer_win1_12889 | 12889 | 12909 | 2516 | TGACCTTGGTGGAACATCCAA | 3498 | AACCTACAAGGTGGTTCCAGT |
| NC_045512.2_21mer_win1_12890 | 12890 | 12910 | 2517 | GACCTTGGTGGAACATCCAAA | 3499 | AAACCTACAAGGTGGTTCCAG |
| NC_045512.2_21mer_win1_12891 | 12891 | 12911 | 2518 | ACCTTGGTGGAACATCCAAAC | 3500 | CAAACCTACAAGGTGGTTCCA |
| NC_045512.2_2lmer_win1_12892 | 12892 | 12912 | 2519 | CCTTGGTGGAACATCCAAACA | 3501 | ACAAACCTACAAGGTGGTTCC |
| NC_045512.2_2lmer_win1_12893 | 12893 | 12913 | 2520 | CTTGGTGGAACATCCAAACAA | 3502 | AACAAACCTACAAGGTGGTTC |
| NC_045512.2_2lmer_win1_12894 | 12894 | 12914 | 2521 | TTGGTGGAACATCCAAACAAT | 3503 | TAACAAACCTACAAGGTGGTT |
| NC_045512.2_2lmer_win1_12895 | 12895 | 12915 | 2522 | TGGTGGAACATCCAAACAATG | 3504 | GTAACAAACCTACAAGGTGGT |
| NC_045512.2_2lmer_win1_12896 | 12896 | 12916 | 2523 | GGTGGAACATCCAAACAATGT | 3505 | TGTAACAAACCTACAAGGTGG |
| NC_045512.2_2lmer_win1_12897 | 12897 | 12917 | 2524 | GTGGAACATCCAAACAATGTC | 3506 | CTGTAACAAACCTACAAGGTG |
| NC_045512.2_2lmer_win1_12898 | 12898 | 12918 | 2525 | TGGAACATCCAAACAATGTCT | 3507 | TCTGTAACAAACCTACAAGGT |
| NC_045512.2_2lmer_win1_12899 | 12899 | 12919 | 2526 | GGAACATCCAAACAATGTCTG | 3508 | GTCTGTAACAAACCTACAAGG |
| NC_045512.2_21mer_win1_12900 | 12900 | 12920 | 2527 | GAACATCCAAACAATGTCTGT | 3509 | TGTCTGTAACAAACCTACAAG |
| NC_045512.2_2lmer_win1_12901 | 12901 | 12921 | 2528 | AACATCCAAACAATGTCTGTG | 3510 | GTGTCTGTAACAAACCTACAA |
| NC_045512.2_2lmer_win1_12902 | 12902 | 12922 | 2529 | ACATCCAAACAATGTCTGTGT | 3511 | TGTGTCTGTAACAAACCTACA |
| NC_045512.2_2lmer_win1_12903 | 12903 | 12923 | 2530 | CATCCAAACAATGTCTGTGTG | 3512 | GTGTGTCTGTAACAAACCTAC |
| NC_045512.2_2lmer_win1_12904 | 12904 | 12924 | 2531 | ATCCAAACAATGTCTGTGTGG | 3513 | GGTGTGTCTGTAACAAACCTA |
| NC_045512.2_2lmer_win1_12966 | 12966 | 12986 | 2532 | ATTTGTTGGATTTATCTCCAT | 3514 | TACCTCTATTTAGGTTGTTTA |
| NC_045512.2_2lmer_win1_12967 | 12967 | 12987 | 2533 | TTTGTTGGATTTATCTCCATA | 3515 | ATACCTCTATTTAGGTTGTTT |
| NC_045512.2_2lmer_win1_12968 | 12968 | 12988 | 2534 | TTGTTGGATTTATCTCCATAC | 3516 | CATACCTCTATTTAGGTTGTT |
| NC_045512.2_2lmer_win1_12969 | 12969 | 12989 | 2535 | TGTTGGATTTATCTCCATACC | 3517 | CCATACCTCTATTTAGGTTGT |
| NC_045512.2_2lmer_win1_12970 | 12970 | 12990 | 2536 | GTTGGATTTATCTCCATACCA | 3518 | ACCATACCTCTATTTAGGTTG |
| NC_045512.2_2lmer_win1_13151 | 13151 | 13171 | 2537 | TTCTACAACACATGTGTGTGA | 3519 | AGTGTGTGTACACAACATCTT |
| NC_045512.2_2lmer_win1_13152 | 13152 | 13172 | 2538 | TCTACAACACATGTGTGTGAC | 3520 | CAGTGTGTGTACACAACATCT |
| NC_045512.2_2lmer_win1_13153 | 13153 | 13173 | 2539 | CTACAACACATGTGTGTGACC | 3521 | CCAGTGTGTGTACACAACATC |
| NC_045512.2_2lmer_win1_13154 | 13154 | 13174 | 2540 | TACAACACATGTGTGTGACCA | 3522 | ACCAGTGTGTGTACACAACAT |
| NC_045512.2_2lmer_win1_13155 | 13155 | 13175 | 2541 | ACAACACATGTGTGTGACCAT | 3523 | TACCAGTGTGTGTACACAACA |
| NC_045512.2_21mer_win1_13156 | 13156 | 13176 | 2542 | CAACACATGTGTGTGACCATG | 3524 | GTACCAGTGTGTGTACACAAC |
| NC_045512.2_21mer_win1_13363 | 13363 | 13383 | 2543 | TTTGTGTCAGACATGGCAGAC | 3525 | CAGACGGTACAGACTGTGTTT |
| NC_045512.2_21mer_win1_13364 | 13364 | 13384 | 2544 | TTGTGTCAGACATGGCAGACG | 3526 | GCAGACGGTACAGACTGTGTT |
| NC_045512.2_21mer_win1_13365 | 13365 | 13385 | 2545 | TGTGTCAGACATGGCAGACGC | 3527 | CGCAGACGGTACAGACTGTGT |
| NC_045512.2_21mer_win1_13366 | 13366 | 13386 | 2546 | GTGTCAGACATGGCAGACGCC | 3528 | CCGCAGACGGTACAGACTGTG |
| NC_045512.2_21mer_win1_13388 | 13388 | 13408 | 2547 | TACACCTTTCCAATACCGACA | 3529 | ACAGCCATAACCTTTCCACAT |
| NC_045512.2_21mer_win1_13389 | 13389 | 13409 | 2548 | ACACCTTTCCAATACCGACAT | 3530 | TACAGCCATAACCTTTCCACA |
| NC_045512.2_21mer_win1_13390 | 13390 | 13410 | 2549 | CACCTTTCCAATACCGACATC | 3531 | CTACAGCCATAACCTTTCCAC |
| NC_045512.2_21mer_win1_13391 | 13391 | 13411 | 2550 | ACCTTTCCAATACCGACATCA | 3532 | ACTACAGCCATAACCTTTCCA |
| NC_045512.2_21mer_win1_13392 | 13392 | 13412 | 2551 | CCTTTCCAATACCGACATCAA | 3533 | AACTACAGCCATAACCTTTCC |
| NC_045512.2_21mer_win1_13393 | 13393 | 13413 | 2552 | CTTTCCAATACCGACATCAAC | 3534 | CAACTACAGCCATAACCTTTC |
| NC_045512.2_21mer_win1_13394 | 13394 | 13414 | 2553 | TTTCCAATACCGACATCAACA | 3535 | ACAACTACAGCCATAACCTTT |
| NC_045512.2_21mer_win1_13395 | 13395 | 13415 | 2554 | TTCCAATACCGACATCAACAC | 3536 | CACAACTACAGCCATAACCTT |
| NC_045512.2_21mer_win1_13396 | 13396 | 13416 | 2555 | TCCAATACCGACATCAACACT | 3537 | TCACAACTACAGCCATAACCT |
| NC_045512.2_21mer_win1_13458 | 13458 | 13478 | 2556 | GCAAAAATTTGCCCAAACGCC | 3538 | CCGCAAACCCGTTTAAAAACG |
| NC_045512.2_21mer_win1_13459 | 13459 | 13479 | 2557 | CAAAAATTTGCCCAAACGCCA | 3539 | ACCGCAAACCCGTTTAAAAAC |
| NC_045512.2_21mer_win1_13460 | 13460 | 13480 | 2558 | AAAAATTTGCCCAAACGCCAC | 3540 | CACCGCAAACCCGTTTAAAAA |
| NC_045512.2_21mer_win1_13461 | 13461 | 13481 | 2559 | AAAATTTGCCCAAACGCCACA | 3541 | ACACCGCAAACCCGTTTAAAA |
| NC_045512.2_21mer_win1_13462 | 13462 | 13482 | 2560 | AAATTTGCCCAAACGCCACAT | 3542 | TACACCGCAAACCCGTTTAAA |
| NC_045512.2_21mer_win1_13463 | 13463 | 13483 | 2561 | AATTTGCCCAAACGCCACATT | 3543 | TTACACCGCAAACCCGTTTAA |
| NC_045512.2_21mer_win1_13464 | 13464 | 13484 | 2562 | ATTTGCCCAAACGCCACATTC | 3544 | CTTACACCGCAAACCCGTTTA |
| NC_045512.2_21mer_win1_13465 | 13465 | 13485 | 2563 | TTTGCCCAAACGCCACATTCA | 3545 | ACTTACACCGCAAACCCGTTT |
| NC_045512.2_21mer_win1_13466 | 13466 | 13486 | 2564 | TTGCCCAAACGCCACATTCAC | 3546 | CACTTACACCGCAAACCCGTT |
| NC_045512.2_21mer_win1_13467 | 13467 | 13487 | 2565 | TGCCCAAACGCCACATTCACG | 3547 | GCACTTACACCGCAAACCCGT |
| NC_045512.2_21mer_win1_13468 | 13468 | 13488 | 2566 | GCCCAAACGCCACATTCACGT | 3548 | TGCACTTACACCGCAAACCCG |
| NC_045512.2_21mer_win1_13469 | 13469 | 13489 | 2567 | CCCAAACGCCACATTCACGTC | 3549 | CTGCACTTACACCGCAAACCC |
| NC_045512.2_21mer_win1_13470 | 13470 | 13490 | 2568 | CCAAACGCCACATTCACGTCG | 3550 | GCTGCACTTACACCGCAAACC |
| NC_045512.2_21mer_win1_13471 | 13471 | 13491 | 2569 | CAAACGCCACATTCACGTCGG | 3551 | GGCTGCACTTACACCGCAAAC |
| NC_045512.2_21mer_win1_13472 | 13472 | 13492 | 2570 | AAACGCCACATTCACGTCGGG | 3552 | GGGCTGCACTTACACCGCAAA |
| NC_045512.2_21mer_win1_13473 | 13473 | 13493 | 2571 | AACGCCACATTCACGTCGGGC | 3553 | CGGGCTGCACTTACACCGCAA |
| NC_045512.2_21mer_win1_13474 | 13474 | 13494 | 2572 | ACGCCACATTCACGTCGGGCA | 3554 | ACGGGCTGCACTTACACCGCA |
| NC_045512.2_21mer_win1_13475 | 13475 | 13495 | 2573 | CGCCACATTCACGTCGGGCAG | 3555 | GACGGGCTGCACTTACACCGC |
| NC_045512.2_21mer_win1_13476 | 13476 | 13496 | 2574 | GCCACATTCACGTCGGGCAGA | 3556 | AGACGGGCTGCACTTACACCG |
| NC_045512.2_21mer_win1_13477 | 13477 | 13497 | 2575 | CCACATTCACGTCGGGCAGAA | 3557 | AAGACGGGCTGCACTTACACC |
| NC_045512.2_21mer_win1_13478 | 13478 | 13498 | 2576 | CACATTCACGTCGGGCAGAAT | 3558 | TAAGACGGGCTGCACTTACAC |
| NC_045512.2_21mer_win1_13479 | 13479 | 13499 | 2577 | ACATTCACGTCGGGCAGAATG | 3559 | GTAAGACGGGCTGCACTTACA |
| NC_045512.2_21mer_win1_13480 | 13480 | 13500 | 2578 | CATTCACGTCGGGCAGAATGT | 3560 | TGTAAGACGGGCTGCACTTAC |
| NC_045512.2_21mer_win1_13481 | 13481 | 13501 | 2579 | ATTCACGTCGGGCAGAATGTG | 3561 | GTGTAAGACGGGCTGCACTTA |
| NC_045512.2_21mer_win1_13482 | 13482 | 13502 | 2580 | TTCACGTCGGGCAGAATGTGG | 3562 | GGTGTAAGACGGGCTGCACTT |
| NC_045512.2_21mer_win1_13483 | 13483 | 13503 | 2581 | TCACGTCGGGCAGAATGTGGC | 3563 | CGGTGTAAGACGGGCTGCACT |
| NC_045512.2_21mer_win1_13484 | 13484 | 13504 | 2582 | CACGTCGGGCAGAATGTGGCA | 3564 | ACGGTGTAAGACGGGCTGCAC |
| NC_045512.2_21mer_win1_13485 | 13485 | 13505 | 2583 | ACGTCGGGCAGAATGTGGCAC | 3565 | CACGGTGTAAGACGGGCTGCA |
| NC_045512.2_21mer_win1_13486 | 13486 | 13506 | 2584 | CGTCGGGCAGAATGTGGCACG | 3566 | GCACGGTGTAAGACGGGCTGC |
| NC_045512.2_21mer_win1_13487 | 13487 | 13507 | 2585 | GTCGGGCAGAATGTGGCACGC | 3567 | CGCACGGTGTAAGACGGGCTG |
| NC_045512.2_21mer_win1_13488 | 13488 | 13508 | 2586 | TCGGGCAGAATGTGGCACGCC | 3568 | CCGCACGGTGTAAGACGGGCT |
| NC_045512.2_21mer_win1_13489 | 13489 | 13509 | 2587 | CGGGCAGAATGTGGCACGCCG | 3569 | GCCGCACGGTGTAAGACGGGC |
| NC_045512.2_21mer_win1_13490 | 13490 | 13510 | 2588 | GGGCAGAATGTGGCACGCCGT | 3570 | TGCCGCACGGTGTAAGACGGG |
| NC_045512.2_21mer_win1_13491 | 13491 | 13511 | 2589 | GGCAGAATGTGGCACGCCGTG | 3571 | GTGCCGCACGGTGTAAGACGG |
| NC_045512.2_21mer_win1_13492 | 13492 | 13512 | 2590 | GCAGAATGTGGCACGCCGTGT | 3572 | TGTGCCGCACGGTGTAAGACG |
| NC_045512.2_21mer_win1_13493 | 13493 | 13513 | 2591 | CAGAATGTGGCACGCCGTGTC | 3573 | CTGTGCCGCACGGTGTAAGAC |
| NC_045512.2_21mer_win1_13494 | 13494 | 13514 | 2592 | AGAATGTGGCACGCCGTGTCC | 3574 | CCTGTGCCGCACGGTGTAAGA |
| NC_045512.2_21mer_win1_13495 | 13495 | 13515 | 2593 | GAATGTGGCACGCCGTGTCCG | 3575 | GCCTGTGCCGCACGGTGTAAG |
| NC_045512.2_21mer_win1_13496 | 13496 | 13516 | 2594 | AATGTGGCACGCCGTGTCCGT | 3576 | TGCCTGTGCCGCACGGTGTAA |
| NC_045512.2_21mer_win1_13497 | 13497 | 13517 | 2595 | ATGTGGCACGCCGTGTCCGTG | 3577 | GTGCCTGTGCCGCACGGTGTA |
| NC_045512.2_21mer_win1_13498 | 13498 | 13518 | 2596 | TGTGGCACGCCGTGTCCGTGA | 3578 | AGTGCCTGTGCCGCACGGTGT |
| NC_045512.2_21mer_win1_13499 | 13499 | 13519 | 2597 | GTGGCACGCCGTGTCCGTGAT | 3579 | TAGTGCCTGTGCCGCACGGTG |
| NC_045512.2_21mer_win1_13500 | 13500 | 13520 | 2598 | TGGCACGCCGTGTCCGTGATC | 3580 | CTAGTGCCTGTGCCGCACGGT |
| NC_045512.2_21mer_win1_13762 | 13762 | 13782 | 2599 | CCACTGTACCATGGTGTATAT | 3581 | TATATGTGGTACCATGTCACC |
| NC_045512.2_21mer_win1_13763 | 13763 | 13783 | 2600 | CACTGTACCATGGTGTATATA | 3582 | ATATATGTGGTACCATGTCAC |
| NC_045512.2_21mer_win1_13764 | 13764 | 13784 | 2601 | ACTGTACCATGGTGTATATAG | 3583 | GATATATGTGGTACCATGTCA |
| NC_045512.2_21mer_win1_13765 | 13765 | 13785 | 2602 | CTGTACCATGGTGTATATAGT | 3584 | TGATATATGTGGTACCATGTC |
| NC_045512.2_21mer_win1_13766 | 13766 | 13786 | 2603 | TGTACCATGGTGTATATAGTG | 3585 | GTGATATATGTGGTACCATGT |
| NC_045512.2_21mer_win1_13767 | 13767 | 13787 | 2604 | GTACCATGGTGTATATAGTGC | 3586 | CGTGATATATGTGGTACCATG |
| NC_045512.2_21mer_win1_13768 | 13768 | 13788 | 2605 | TACCATGGTGTATATAGTGCA | 3587 | ACGTGATATATGTGGTACCAT |
| NC_045512.2_21mer_win1_13769 | 13769 | 13789 | 2606 | ACCATGGTGTATATAGTGCAG | 3588 | GACGTGATATATGTGGTACCA |
| NC_045512.2_21mer_win1_13770 | 13770 | 13790 | 2607 | CCATGGTGTATATAGTGCAGT | 3589 | TGACGTGATATATGTGGTACC |
| NC_045512.2_21mer_win1_14290 | 14290 | 14310 | 2608 | CTGGCAATAAAATTTATAACC | 3590 | CCAATATTTAAAATAACGGTC |
| NC_045512.2_21mer_win1_14291 | 14291 | 14311 | 2609 | TGGCAATAAAATTTATAACCC | 3591 | CCCAATATTTAAAATAACGGT |
| NC_045512.2_21mer_win1_14292 | 14292 | 14312 | 2610 | GGCAATAAAATTTATAACCCT | 3592 | TCCCAATATTTAAAATAACGG |
| NC_045512.2_21mer_win1_14404 | 14404 | 14424 | 2611 | GGTGGATGTTCAAAACCTGGT | 3593 | TGGTCCAAAACTTGTAGGTGG |
| NC_045512.2_2lmer_win1_14405 | 14405 | 14425 | 2612 | GTGGATGTTCAAAACCTGGTG | 3594 | GTGGTCCAAAACTTGTAGGTG |
| NC_045512.2_21mer_win1_14406 | 14406 | 14426 | 2613 | TGGATGTTCAAAACCTGGTGA | 3595 | AGTGGTCCAAAACTTGTAGGT |
| NC_045512.2_21mer_win1_14407 | 14407 | 14427 | 2614 | GGATGTTCAAAACCTGGTGAT | 3596 | TAGTGGTCCAAAACTTGTAGG |
| NC_045512.2_2lmer_win1_14408 | 14408 | 14428 | 2615 | GATGTTCAAAACCTGGTGATC | 3597 | CTAGTGGTCCAAAACTTGTAG |
| NC_045512.2_2lmer_win1_14409 | 14409 | 14429 | 2616 | ATGTTCAAAACCTGGTGATCA | 3598 | ACTAGTGGTCCAAAACTTGTA |
| NC_045512.2_2lmer_win1_14500 | 14500 | 14520 | 2617 | CATGTATTAGTCCTACATTTG | 3599 | GTTTACATCCTGATTATGTAC |
| NC_045512.2_2lmer_win1_14501 | 14501 | 14521 | 2618 | ATGTATTAGTCCTACATTTGA | 3600 | AGTTTACATCCTGATTATGTA |
| NC_045512.2_2lmer_win1_14502 | 14502 | 14522 | 2619 | TGTATTAGTCCTACATTTGAA | 3601 | AAGTTTACATCCTGATTATGT |
| NC_045512.2_2lmer_win1_14503 | 14503 | 14523 | 2620 | GTATTAGTCCTACATTTGAAT | 3602 | TAAGTTTACATCCTGATTATG |
| NC_045512.2_2lmer_win1_14504 | 14504 | 14524 | 2621 | TATTAGTCCTACATTTGAATG | 3603 | GTAAGTTTACATCCTGATTAT |
| NC_045512.2_2lmer_win1_14505 | 14505 | 14525 | 2622 | ATTAGTCCTACATTTGAATGT | 3604 | TGTAAGTTTACATCCTGATTA |
| NC_045512.2_2lmer_win1_14506 | 14506 | 14526 | 2623 | TTAGTCCTACATTTGAATGTA | 3605 | ATGTAAGTTTACATCCTGATT |
| NC_045512.2_2lmer_win1_14507 | 14507 | 14527 | 2624 | TAGTCCTACATTTGAATGTAT | 3606 | TATGTAAGTTTACATCCTGAT |
| NC_045512.2_2lmer_win1_14508 | 14508 | 14528 | 2625 | AGTCCTACATTTGAATGTATC | 3607 | CTATGTAAGTTTACATCCTGA |
| NC_045512.2_2lmer_win1_14509 | 14509 | 14529 | 2626 | GTCCTACATTTGAATGTATCG | 3608 | GCTATGTAAGTTTACATCCTG |
| NC_045512.2_2lmer_win1_14510 | 14510 | 14530 | 2627 | TCCTACATTTGAATGTATCGA | 3609 | AGCTATGTAAGTTTACATCCT |
| NC_045512.2_2lmer_win1_14511 | 14511 | 14531 | 2628 | CCTACATTTGAATGTATCGAG | 3610 | GAGCTATGTAAGTTTACATCC |
| NC_045512.2_2lmer_win1_14650 | 14650 | 14670 | 2629 | TTACAACGAAAAGTTTGACAG | 3611 | GACAGTTTGAAAAGCAACATT |
| NC_045512.2_2lmer_win1_14651 | 14651 | 14671 | 2630 | TACAACGAAAAGTTTGACAGT | 3612 | TGACAGTTTGAAAAGCAACAT |
| NC_045512.2_2lmer_win1_14652 | 14652 | 14672 | 2631 | ACAACGAAAAGTTTGACAGTT | 3613 | TTGACAGTTTGAAAAGCAACA |
| NC_045512.2_2lmer_win1_14653 | 14653 | 14673 | 2632 | CAACGAAAAGTTTGACAGTTT | 3614 | TTTGACAGTTTGAAAAGCAAC |
| NC_045512.2_2lmer_win1_14654 | 14654 | 14674 | 2633 | AACGAAAAGTTTGACAGTTTG | 3615 | GTTTGACAGTTTGAAAAGCAA |
| NC_045512.2_21mer_win1_14655 | 14655 | 14675 | 2634 | ACGAAAAGTTTGACAGTTTGG | 3616 | GGTTTGACAGTTTGAAAAGCA |
| NC_045512.2_21mer_win1_14656 | 14656 | 14676 | 2635 | CGAAAAGTTTGACAGTTTGGG | 3617 | GGGTTTGACAGTTTGAAAAGC |
| NC_045512.2_21mer_win1_14657 | 14657 | 14677 | 2636 | GAAAAGTTTGACAGTTTGGGC | 3618 | CGGGTTTGACAGTTTGAAAAG |
| NC_045512.2_21mer_win1_14658 | 14658 | 14678 | 2637 | AAAAGTTTGACAGTTTGGGCC | 3619 | CCGGGTTTGACAGTTTGAAAA |
| NC_045512.2_21mer_win1_14659 | 14659 | 14679 | 2638 | AAAGTTTGACAGTTTGGGCCA | 3620 | ACCGGGTTTGACAGTTTGAAA |
| NC_045512.2_21mer_win1_14660 | 14660 | 14680 | 2639 | AAGTTTGACAGTTTGGGCCAT | 3621 | TACCGGGTTTGACAGTTTGAA |
| NC_045512.2_21mer_win1_14661 | 14661 | 14681 | 2640 | AGTTTGACAGTTTGGGCCATT | 3622 | TTACCGGGTTTGACAGTTTGA |
| NC_045512.2_21mer_win1_14662 | 14662 | 14682 | 2641 | GTTTGACAGTTTGGGCCATTA | 3623 | ATTACCGGGTTTGACAGTTTG |
| NC_045512.2_21mer_win1_14663 | 14663 | 14683 | 2642 | TTTGACAGTTTGGGCCATTAA | 3624 | AATTACCGGGTTTGACAGTTT |
| NC_045512.2_21mer_win1_14664 | 14664 | 14684 | 2643 | TTGACAGTTTGGGCCATTAAA | 3625 | AAATTACCGGGTTTGACAGTT |
| NC_045512.2_21mer_win1_14665 | 14665 | 14685 | 2644 | TGACAGTTTGGGCCATTAAAA | 3626 | AAAATTACCGGGTTTGACAGT |
| NC_045512.2_21mer_win1_14666 | 14666 | 14686 | 2645 | GACAGTTTGGGCCATTAAAAT | 3627 | TAAAATTACCGGGTTTGACAG |
| NC_045512.2_21mer_win1_14667 | 14667 | 14687 | 2646 | ACAGTTTGGGCCATTAAAATT | 3628 | TTAAAATTACCGGGTTTGACA |
| NC_045512.2_21mer_win1_14722 | 14722 | 14742 | 2647 | AAGAAATTCCTTCCTTCAAGA | 3629 | AGAACTTCCTTCCTTAAAGAA |
| NC_045512.2_21mer_win1_14723 | 14723 | 14743 | 2648 | AGAAATTCCTTCCTTCAAGAC | 3630 | CAGAACTTCCTTCCTTAAAGA |
| NC_045512.2_21mer_win1_14724 | 14724 | 14744 | 2649 | GAAATTCCTTCCTTCAAGACA | 3631 | ACAGAACTTCCTTCCTTAAAG |
| NC_045512.2_21mer_win1_14725 | 14725 | 14745 | 2650 | AAATTCCTTCCTTCAAGACAA | 3632 | AACAGAACTTCCTTCCTTAAA |
| NC_045512.2_21mer_win1_14726 | 14726 | 14746 | 2651 | AATTCCTTCCTTCAAGACAAC | 3633 | CAACAGAACTTCCTTCCTTAA |
| NC_045512.2_21mer_win1_14727 | 14727 | 14747 | 2652 | ATTCCTTCCTTCAAGACAACT | 3634 | TCAACAGAACTTCCTTCCTTA |
| NC_045512.2_21mer_win1_14728 | 14728 | 14748 | 2653 | TTCCTTCCTTCAAGACAACTT | 3635 | TTCAACAGAACTTCCTTCCTT |
| NC_045512.2_21mer_win1_14750 | 14750 | 14770 | 2654 | ATTTTGTGAAGAAGAAACGAG | 3636 | GAGCAAAGAAGAAGTGTTTTA |
| NC_045512.2_21mer_win1_14751 | 14751 | 14771 | 2655 | TTTTGTGAAGAAGAAACGAGT | 3637 | TGAGCAAAGAAGAAGTGTTTT |
| NC_045512.2_21mer_win1_14752 | 14752 | 14772 | 2656 | TTTGTGAAGAAGAAACGAGTC | 3638 | CTGAGCAAAGAAGAAGTGTTT |
| NC_045512.2_21mer_win1_14753 | 14753 | 14773 | 2657 | TTGTGAAGAAGAAACGAGTCC | 3639 | CCTGAGCAAAGAAGAAGTGTT |
| NC_045512.2_21mer_win1_14754 | 14754 | 14774 | 2658 | TGTGAAGAAGAAACGAGTCCT | 3640 | TCCTGAGCAAAGAAGAAGTGT |
| NC_045512.2_21mer_win1_14755 | 14755 | 14775 | 2659 | GTGAAGAAGAAACGAGTCCTA | 3641 | ATCCTGAGCAAAGAAGAAGTG |
| NC_045512.2_21mer_win1_14756 | 14756 | 14776 | 2660 | TGAAGAAGAAACGAGTCCTAC | 3642 | CATCCTGAGCAAAGAAGAAGT |
| NC_045512.2_21mer_win1_14757 | 14757 | 14777 | 2661 | GAAGAAGAAACGAGTCCTACC | 3643 | CCATCCTGAGCAAAGAAGAAG |
| NC_045512.2_21mer_win1_14821 | 14821 | 14841 | 2662 | GGTTGTTACACACTATAGTCT | 3644 | TCTGATATCACACATTGTTGG |
| NC_045512.2_21mer_win1_14822 | 14822 | 14842 | 2663 | GTTGTTACACACTATAGTCTG | 3645 | GTCTGATATCACACATTGTTG |
| NC_045512.2_21mer_win1_14823 | 14823 | 14843 | 2664 | TTGTTACACACTATAGTCTGT | 3646 | TGTCTGATATCACACATTGTT |
| NC_045512.2_21mer_win1_14824 | 14824 | 14844 | 2665 | TGTTACACACTATAGTCTGTT | 3647 | TTGTCTGATATCACACATTGT |
| NC_045512.2_21mer_win1_14825 | 14825 | 14845 | 2666 | GTTACACACTATAGTCTGTTG | 3648 | GTTGTCTGATATCACACATTG |
| NC_045512.2_21mer_win1_14826 | 14826 | 14846 | 2667 | TTACACACTATAGTCTGTTGA | 3649 | AGTTGTCTGATATCACACATT |
| NC_045512.2_21mer_win1_14875 | 14875 | 14895 | 2668 | ATGAAACTAACAATGCTACCA | 3650 | ACCATCGTAACAATCAAAGTA |
| NC_045512.2_21mer_win1_14876 | 14876 | 14896 | 2669 | TGAAACTAACAATGCTACCAC | 3651 | CACCATCGTAACAATCAAAGT |
| NC_045512.2_21mer_win1_14877 | 14877 | 14897 | 2670 | GAAACTAACAATGCTACCACC | 3652 | CCACCATCGTAACAATCAAAG |
| NC_045512.2_21mer_win1_14878 | 14878 | 14898 | 2671 | AAACTAACAATGCTACCACCG | 3653 | GCCACCATCGTAACAATCAAA |
| NC_045512.2_21mer_win1_14879 | 14879 | 14899 | 2672 | AACTAACAATGCTACCACCGA | 3654 | AGCCACCATCGTAACAATCAA |
| NC_045512.2_21mer_win1_14880 | 14880 | 14900 | 2673 | ACTAACAATGCTACCACCGAC | 3655 | CAGCCACCATCGTAACAATCA |
| NC_045512.2_21mer_win1_14881 | 14881 | 14901 | 2674 | CTAACAATGCTACCACCGACA | 3656 | ACAGCCACCATCGTAACAATC |
| NC_045512.2_21mer_win1_14882 | 14882 | 14902 | 2675 | TAACAATGCTACCACCGACAT | 3657 | TACAGCCACCATCGTAACAAT |
| NC_045512.2_21mer_win1_14883 | 14883 | 14903 | 2676 | AACAATGCTACCACCGACATA | 3658 | ATACAGCCACCATCGTAACAA |
| NC_045512.2_21mer_win1_14962 | 14962 | 14982 | 2677 | TTTACCCCATTCCGATCTGAA | 3659 | AAGTCTAGCCTTACCCCATTT |
| NC_045512.2_21mer_win1_14963 | 14963 | 14983 | 2678 | TTACCCCATTCCGATCTGAAA | 3660 | AAAGTCTAGCCTTACCCCATT |
| NC_045512.2_21mer_win1_14964 | 14964 | 14984 | 2679 | TACCCCATTCCGATCTGAAAT | 3661 | TAAAGTCTAGCCTTACCCCAT |
| NC_045512.2_21mer_win1_14965 | 14965 | 14985 | 2680 | ACCCCATTCCGATCTGAAATA | 3662 | ATAAAGTCTAGCCTTACCCCA |
| NC_045512.2_21mer_win1_14966 | 14966 | 14986 | 2681 | CCCCATTCCGATCTGAAATAA | 3663 | AATAAAGTCTAGCCTTACCCC |
| NC_045512.2_21mer_win1_14967 | 14967 | 14987 | 2682 | CCCATTCCGATCTGAAATAAT | 3664 | TAATAAAGTCTAGCCTTACCC |
| NC_045512.2_21mer_win1_14968 | 14968 | 14988 | 2683 | CCATTCCGATCTGAAATAATA | 3665 | ATAATAAAGTCTAGCCTTACC |
| NC_045512.2_21mer_win1_14969 | 14969 | 14989 | 2684 | CATTCCGATCTGAAATAATAC | 3666 | CATAATAAAGTCTAGCCTTAC |
| NC_045512.2_21mer_win1_14970 | 14970 | 14990 | 2685 | ATTCCGATCTGAAATAATACT | 3667 | TCATAATAAAGTCTAGCCTTA |
| NC_045512.2_21mer_win1_14992 | 14992 | 15012 | 2686 | AGTTACTCAATACTCCTAGTT | 3668 | TTGATCCTCATAACTCATTGA |
| NC_045512.2_21mer_win1_14993 | 14993 | 15013 | 2687 | GTTACTCAATACTCCTAGTTC | 3669 | CTTGATCCTCATAACTCATTG |
| NC_045512.2_21mer_win1_14994 | 14994 | 15014 | 2688 | TTACTCAATACTCCTAGTTCT | 3670 | TCTTGATCCTCATAACTCATT |
| NC_045512.2_21mer_win1_14995 | 14995 | 15015 | 2689 | TACTCAATACTCCTAGTTCTA | 3671 | ATCTTGATCCTCATAACTCAT |
| NC_045512.2_21mer_win1_14996 | 14996 | 15016 | 2690 | ACTCAATACTCCTAGTTCTAC | 3672 | CATCTTGATCCTCATAACTCA |
| NC_045512.2_21mer_win1_14997 | 14997 | 15017 | 2691 | CTCAATACTCCTAGTTCTACG | 3673 | GCATCTTGATCCTCATAACTC |
| NC_045512.2_21mer_win1_14998 | 14998 | 15018 | 2692 | TCAATACTCCTAGTTCTACGT | 3674 | TGCATCTTGATCCTCATAACT |
| NC_045512.2_21mer_win1_14999 | 14999 | 15019 | 2693 | CAATACTCCTAGTTCTACGTG | 3675 | GTGCATCTTGATCCTCATAAC |
| NC_045512.2_21mer_win1_15000 | 15000 | 15020 | 2694 | AATACTCCTAGTTCTACGTGA | 3676 | AGTGCATCTTGATCCTCATAA |
| NC_045512.2_21mer_win1_15055 | 15055 | 15075 | 2695 | TATTGAGTTTACTTAGAATTC | 3677 | CTTAAGATTCATTTGAGTTAT |
| NC_045512.2_21mer_win1_15056 | 15056 | 15076 | 2696 | ATTGAGTTTACTTAGAATTCA | 3678 | ACTTAAGATTCATTTGAGTTA |
| NC_045512.2_21mer_win1_15057 | 15057 | 15077 | 2697 | TTGAGTTTACTTAGAATTCAT | 3679 | TACTTAAGATTCATTTGAGTT |
| NC_045512.2_21mer_win1_15058 | 15058 | 15078 | 2698 | TGAGTTTACTTAGAATTCATA | 3680 | ATACTTAAGATTCATTTGAGT |
| NC_045512.2_21mer_win1_15059 | 15059 | 15079 | 2699 | GAGTTTACTTAGAATTCATAC | 3681 | CATACTTAAGATTCATTTGAG |
| NC_045512.2_21mer_win1_15060 | 15060 | 15080 | 2700 | AGTTTACTTAGAATTCATACG | 3682 | GCATACTTAAGATTCATTTGA |
| NC_045512.2_21mer_win1_15061 | 15061 | 15081 | 2701 | GTTTACTTAGAATTCATACGG | 3683 | GGCATACTTAAGATTCATTTG |
| NC_045512.2_21mer_win1_15062 | 15062 | 15082 | 2702 | TTTACTTAGAATTCATACGGT | 3684 | TGGCATACTTAAGATTCATTT |
| NC_045512.2_2lmer_win1_15063 | 15063 | 15083 | 2703 | TTACTTAGAATTCATACGGTA | 3685 | ATGGCATACTTAAGATTCATT |
| NC_045512.2_2lmer_win1_15064 | 15064 | 15084 | 2704 | TACTTAGAATTCATACGGTAA | 3686 | AATGGCATACTTAAGATTCAT |
| NC_045512.2_2lmer_win1_15065 | 15065 | 15085 | 2705 | ACTTAGAATTCATACGGTAAT | 3687 | TAATGGCATACTTAAGATTCA |
| NC_045512.2_2lmer_win1_15066 | 15066 | 15086 | 2706 | CTTAGAATTCATACGGTAATC | 3688 | CTAATGGCATACTTAAGATTC |
| NC_045512.2_2lmer_win1_15067 | 15067 | 15087 | 2707 | TTAGAATTCATACGGTAATCA | 3689 | ACTAATGGCATACTTAAGATT |
| NC_045512.2_2lmer_win1_15068 | 15068 | 15088 | 2708 | TAGAATTCATACGGTAATCAC | 3690 | CACTAATGGCATACTTAAGAT |
| NC_045512.2_2lmer_win1_15069 | 15069 | 15089 | 2709 | AGAATTCATACGGTAATCACG | 3691 | GCACTAATGGCATACTTAAGA |
| NC_045512.2_2lmer_win1_15070 | 15070 | 15090 | 2710 | GAATTCATACGGTAATCACGT | 3692 | TGCACTAATGGCATACTTAAG |
| NC_045512.2_2lmer_win1_15071 | 15071 | 15091 | 2711 | AATTCATACGGTAATCACGTT | 3693 | TTGCACTAATGGCATACTTAA |
| NC_045512.2_2lmer_win1_15072 | 15072 | 15092 | 2712 | ATTCATACGGTAATCACGTTT | 3694 | TTTGCACTAATGGCATACTTA |
| NC_045512.2_2lmer_win1_15073 | 15073 | 15093 | 2713 | TTCATACGGTAATCACGTTTC | 3695 | CTTTGCACTAATGGCATACTT |
| NC_045512.2_2lmer_win1_15074 | 15074 | 15094 | 2714 | TCATACGGTAATCACGTTTCT | 3696 | TCTTTGCACTAATGGCATACT |
| NC_045512.2_2lmer_win1_15075 | 15075 | 15095 | 2715 | CATACGGTAATCACGTTTCTT | 3697 | TTCTTTGCACTAATGGCATAC |
| NC_045512.2_2lmer_win1_15076 | 15076 | 15096 | 2716 | ATACGGTAATCACGTTTCTTA | 3698 | ATTCTTTGCACTAATGGCATA |
| NC_045512.2_2lmer_win1_15077 | 15077 | 15097 | 2717 | TACGGTAATCACGTTTCTTAT | 3699 | TATTCTTTGCACTAATGGCAT |
| NC_045512.2_2lmer_win1_15078 | 15078 | 15098 | 2718 | ACGGTAATCACGTTTCTTATC | 3700 | CTATTCTTTGCACTAATGGCA |
| NC_045512.2_2lmer_win1_15079 | 15079 | 15099 | 2719 | CGGTAATCACGTTTCTTATCT | 3701 | TCTATTCTTTGCACTAATGGC |
| NC_045512.2_2lmer_win1_15080 | 15080 | 15100 | 2720 | GGTAATCACGTTTCTTATCTC | 3702 | CTCTATTCTTTGCACTAATGG |
| NC_045512.2_2lmer_win1_15081 | 15081 | 15101 | 2721 | GTAATCACGTTTCTTATCTCG | 3703 | GCTCTATTCTTTGCACTAATG |
| NC_045512.2_2lmer_win1_15082 | 15082 | 15102 | 2722 | TAATCACGTTTCTTATCTCGA | 3704 | AGCTCTATTCTTTGCACTAAT |
| NC_045512.2_2lmer_win1_15083 | 15083 | 15103 | 2723 | AATCACGTTTCTTATCTCGAG | 3705 | GAGCTCTATTCTTTGCACTAA |
| NC_045512.2_2lmer_win1_15084 | 15084 | 15104 | 2724 | ATCACGTTTCTTATCTCGAGC | 3706 | CGAGCTCTATTCTTTGCACTA |
| NC_045512.2_2lmer_win1_15085 | 15085 | 15105 | 2725 | TCACGTTTCTTATCTCGAGCG | 3707 | GCGAGCTCTATTCTTTGCACT |
| NC_045512.2_21mer_win1_15086 | 15086 | 15106 | 2726 | CACGTTTCTTATCTCGAGCGT | 3708 | TGCGAGCTCTATTCTTTGCAC |
| NC_045512.2_21mer_win1_15087 | 15087 | 15107 | 2727 | ACGTTTCTTATCTCGAGCGTG | 3709 | GTGCGAGCTCTATTCTTTGCA |
| NC_045512.2_21mer_win1_15088 | 15088 | 15108 | 2728 | CGTTTCTTATCTCGAGCGTGG | 3710 | GGTGCGAGCTCTATTCTTTGC |
| NC_045512.2_21mer_win1_15089 | 15089 | 15109 | 2729 | GTTTCTTATCTCGAGCGTGGC | 3711 | CGGTGCGAGCTCTATTCTTTG |
| NC_045512.2_21mer_win1_15090 | 15090 | 15110 | 2730 | TTTCTTATCTCGAGCGTGGCA | 3712 | ACGGTGCGAGCTCTATTCTTT |
| NC_045512.2_21mer_win1_15091 | 15091 | 15111 | 2731 | TTCTTATCTCGAGCGTGGCAT | 3713 | TACGGTGCGAGCTCTATTCTT |
| NC_045512.2_21mer_win1_15092 | 15092 | 15112 | 2732 | TCTTATCTCGAGCGTGGCATC | 3714 | CTACGGTGCGAGCTCTATTCT |
| NC_045512.2_21mer_win1_15093 | 15093 | 15113 | 2733 | CTTATCTCGAGCGTGGCATCG | 3715 | GCTACGGTGCGAGCTCTATTC |
| NC_045512.2_21mer_win1_15094 | 15094 | 15114 | 2734 | TTATCTCGAGCGTGGCATCGA | 3716 | AGCTACGGTGCGAGCTCTATT |
| NC_045512.2_21mer_win1_15095 | 15095 | 15115 | 2735 | TATCTCGAGCGTGGCATCGAC | 3717 | CAGCTACGGTGCGAGCTCTAT |
| NC_045512.2_21mer_win1_15096 | 15096 | 15116 | 2736 | ATCTCGAGCGTGGCATCGACC | 3718 | CCAGCTACGGTGCGAGCTCTA |
| NC_045512.2_21mer_win1_15097 | 15097 | 15117 | 2737 | TCTCGAGCGTGGCATCGACCA | 3719 | ACCAGCTACGGTGCGAGCTCT |
| NC_045512.2_21mer_win1_15098 | 15098 | 15118 | 2738 | CTCGAGCGTGGCATCGACCAC | 3720 | CACCAGCTACGGTGCGAGCTC |
| NC_045512.2_21mer_win1_15099 | 15099 | 15119 | 2739 | TCGAGCGTGGCATCGACCACA | 3721 | ACACCAGCTACGGTGCGAGCT |
| NC_045512.2_21mer_win1_15100 | 15100 | 15120 | 2740 | CGAGCGTGGCATCGACCACAG | 3722 | GACACCAGCTACGGTGCGAGC |
| NC_045512.2_21mer_win1_15101 | 15101 | 15121 | 2741 | GAGCGTGGCATCGACCACAGA | 3723 | AGACACCAGCTACGGTGCGAG |
| NC_045512.2_21mer_win1_15102 | 15102 | 15122 | 2742 | AGCGTGGCATCGACCACAGAG | 3724 | GAGACACCAGCTACGGTGCGA |
| NC_045512.2_21mer_win1_15103 | 15103 | 15123 | 2743 | GCGTGGCATCGACCACAGAGA | 3725 | AGAGACACCAGCTACGGTGCG |
| NC_045512.2_21mer_win1_15104 | 15104 | 15124 | 2744 | CGTGGCATCGACCACAGAGAT | 3726 | TAGAGACACCAGCTACGGTGC |
| NC_045512.2_21mer_win1_15105 | 15105 | 15125 | 2745 | GTGGCATCGACCACAGAGATA | 3727 | ATAGAGACACCAGCTACGGTG |
| NC_045512.2_21mer_win1_15106 | 15106 | 15126 | 2746 | TGGCATCGACCACAGAGATAG | 3728 | GATAGAGACACCAGCTACGGT |
| NC_045512.2_21mer_win1_15107 | 15107 | 15127 | 2747 | GGCATCGACCACAGAGATAGA | 3729 | AGATAGAGACACCAGCTACGG |
| NC_045512.2_21mer_win1_15108 | 15108 | 15128 | 2748 | GCATCGACCACAGAGATAGAC | 3730 | CAGATAGAGACACCAGCTACG |
| NC_045512.2_2lmer_win1_15109 | 15109 | 15129 | 2749 | CATCGACCACAGAGATAGACA | 3731 | ACAGATAGAGACACCAGCTAC |
| NC_045512.2_2lmer_win1_15110 | 15110 | 15130 | 2750 | ATCGACCACAGAGATAGACAT | 3732 | TACAGATAGAGACACCAGCTA |
| NC_045512.2_2lmer_win1_15111 | 15111 | 15131 | 2751 | TCGACCACAGAGATAGACATC | 3733 | CTACAGATAGAGACACCAGCT |
| NC_045512.2_2lmer_win1_15112 | 15112 | 15132 | 2752 | CGACCACAGAGATAGACATCA | 3734 | ACTACAGATAGAGACACCAGC |
| NC_045512.2_2lmer_win1_15113 | 15113 | 15133 | 2753 | GACCACAGAGATAGACATCAT | 3735 | TACTACAGATAGAGACACCAG |
| NC_045512.2_2lmer_win1_15114 | 15114 | 15134 | 2754 | ACCACAGAGATAGACATCATG | 3736 | GTACTACAGATAGAGACACCA |
| NC_045512.2_2lmer_win1_15115 | 15115 | 15135 | 2755 | CCACAGAGATAGACATCATGA | 3737 | AGTACTACAGATAGAGACACC |
| NC_045512.2_2lmer_win1_15116 | 15116 | 15136 | 2756 | CACAGAGATAGACATCATGAT | 3738 | TAGTACTACAGATAGAGACAC |
| NC_045512.2_2lmer_win1_15117 | 15117 | 15137 | 2757 | ACAGAGATAGACATCATGATA | 3739 | ATAGTACTACAGATAGAGACA |
| NC_045512.2_2lmer_win1_15118 | 15118 | 15138 | 2758 | CAGAGATAGACATCATGATAC | 3740 | CATAGTACTACAGATAGAGAC |
| NC_045512.2_2lmer_win1_15119 | 15119 | 15139 | 2759 | AGAGATAGACATCATGATACT | 3741 | TCATAGTACTACAGATAGAGA |
| NC_045512.2_2lmer_win1_15120 | 15120 | 15140 | 2760 | GAGATAGACATCATGATACTG | 3742 | GTCATAGTACTACAGATAGAG |
| NC_045512.2_2lmer_win1_15172 | 15172 | 15192 | 2761 | AGTTATCGGCGGTGATCTCCT | 3743 | TCCTCTAGTGGCGGCTATTGA |
| NC_045512.2_2lmer_win1_15173 | 15173 | 15193 | 2762 | GTTATCGGCGGTGATCTCCTC | 3744 | CTCCTCTAGTGGCGGCTATTG |
| NC_045512.2_2lmer_win1_15174 | 15174 | 15194 | 2763 | TTATCGGCGGTGATCTCCTCG | 3745 | GCTCCTCTAGTGGCGGCTATT |
| NC_045512.2_2lmer_win1_15175 | 15175 | 15195 | 2764 | TATCGGCGGTGATCTCCTCGA | 3746 | AGCTCCTCTAGTGGCGGCTAT |
| NC_045512.2_2lmer_win1_15176 | 15176 | 15196 | 2765 | ATCGGCGGTGATCTCCTCGAT | 3747 | TAGCTCCTCTAGTGGCGGCTA |
| NC_045512.2_2lmer_win1_15177 | 15177 | 15197 | 2766 | TCGGCGGTGATCTCCTCGATG | 3748 | GTAGCTCCTCTAGTGGCGGCT |
| NC_045512.2_2lmer_win1_15178 | 15178 | 15198 | 2767 | CGGCGGTGATCTCCTCGATGA | 3749 | AGTAGCTCCTCTAGTGGCGGC |
| NC_045512.2_2lmer_win1_15179 | 15179 | 15199 | 2768 | GGCGGTGATCTCCTCGATGAC | 3750 | CAGTAGCTCCTCTAGTGGCGG |
| NC_045512.2_2lmer_win1_15180 | 15180 | 15200 | 2769 | GCGGTGATCTCCTCGATGACA | 3751 | ACAGTAGCTCCTCTAGTGGCG |
| NC_045512.2_2lmer_win1_15310 | 15310 | 15330 | 2770 | TCTCGGTACGGATTGTACGAA | 3752 | AAGCATGTTAGGCATGGCTCT |
| NC_045512.2_2lmer_win1_15311 | 15311 | 15331 | 2771 | CTCGGTACGGATTGTACGAAT | 3753 | TAAGCATGTTAGGCATGGCTC |
| NC_045512.2_21mer_win1_15312 | 15312 | 15332 | 2772 | TCGGTACGGATTGTACGAATC | 3754 | CTAAGCATGTTAGGCATGGCT |
| NC_045512.2_21mer_win1_15346 | 15346 | 15366 | 2773 | GAACAAGAACGAGCGTTTGTA | 3755 | ATGTTTGCGAGCAAGAACAAG |
| NC_045512.2_21mer_win1_15347 | 15347 | 15367 | 2774 | AACAAGAACGAGCGTTTGTAT | 3756 | TATGTTTGCGAGCAAGAACAA |
| NC_045512.2_21mer_win1_15496 | 15496 | 15516 | 2775 | TGTTGACGAATACGATTATCA | 3757 | ACTATTAGCATAAGCAGTTGT |
| NC_045512.2_21mer_win1_15497 | 15497 | 15517 | 2776 | GTTGACGAATACGATTATCAC | 3758 | CACTATTAGCATAAGCAGTTG |
| NC_045512.2_21mer_win1_15498 | 15498 | 15518 | 2777 | TTGACGAATACGATTATCACA | 3759 | ACACTATTAGCATAAGCAGTT |
| NC_045512.2_21mer_win1_15622 | 15622 | 15642 | 2778 | ATACTCACAGAGATATCTTTA | 3760 | ATTTCTATAGAGACACTCATA |
| NC_045512.2_21mer_win1_15623 | 15623 | 15643 | 2779 | TACTCACAGAGATATCTTTAT | 3761 | TATTTCTATAGAGACACTCAT |
| NC_045512.2_21mer_win1_15624 | 15624 | 15644 | 2780 | ACTCACAGAGATATCTTTATC | 3762 | CTATTTCTATAGAGACACTCA |
| NC_045512.2_21mer_win1_15838 | 15838 | 15858 | 2781 | ACCTGACTCTGACTGGAATGA | 3763 | AGTAAGGTCAGTCTCAGTCCA |
| NC_045512.2_21mer_win1_15839 | 15839 | 15859 | 2782 | CCTGACTCTGACTGGAATGAT | 3764 | TAGTAAGGTCAGTCTCAGTCC |
| NC_045512.2_21mer_win1_15840 | 15840 | 15860 | 2783 | CTGACTCTGACTGGAATGATT | 3765 | TTAGTAAGGTCAGTCTCAGTC |
| NC_045512.2_21mer_win1_15841 | 15841 | 15861 | 2784 | TGACTCTGACTGGAATGATTT | 3766 | TTTAGTAAGGTCAGTCTCAGT |
| NC_045512.2_21mer_win1_15842 | 15842 | 15862 | 2785 | GACTCTGACTGGAATGATTTC | 3767 | CTTTAGTAAGGTCAGTCTCAG |
| NC_045512.2_21mer_win1_15843 | 15843 | 15863 | 2786 | ACTCTGACTGGAATGATTTCC | 3768 | CCTTTAGTAAGGTCAGTCTCA |
| NC_045512.2_21mer_win1_15844 | 15844 | 15864 | 2787 | CTCTGACTGGAATGATTTCCT | 3769 | TCCTTTAGTAAGGTCAGTCTC |
| NC_045512.2_21mer_win1_15845 | 15845 | 15865 | 2788 | TCTGACTGGAATGATTTCCTG | 3770 | GTCCTTTAGTAAGGTCAGTCT |
| NC_045512.2_21mer_win1_15846 | 15846 | 15866 | 2789 | CTGACTGGAATGATTTCCTGG | 3771 | GGTCCTTTAGTAAGGTCAGTC |
| NC_045512.2_21mer_win1_15847 | 15847 | 15867 | 2790 | TGACTGGAATGATTTCCTGGA | 3772 | AGGTCCTTTAGTAAGGTCAGT |
| NC_045512.2_21mer_win1_15848 | 15848 | 15868 | 2791 | GACTGGAATGATTTCCTGGAG | 3773 | GAGGTCCTTTAGTAAGGTCAG |
| NC_045512.2_21mer_win1_15849 | 15849 | 15869 | 2792 | ACTGGAATGATTTCCTGGAGT | 3774 | TGAGGTCCTTTAGTAAGGTCA |
| NC_045512.2_21mer_win1_15985 | 15985 | 16005 | 2793 | TTTTGTCTACCATGTGAATAC | 3775 | CATAAGTGTACCATCTGTTTT |
| NC_045512.2_21mer_win1_15986 | 15986 | 16006 | 2794 | TTTGTCTACCATGTGAATACT | 3776 | TCATAAGTGTACCATCTGTTT |
| NC_045512.2_21mer_win1_15987 | 15987 | 16007 | 2795 | TTGTCTACCATGTGAATACTA | 3777 | ATCATAAGTGTACCATCTGTT |
| NC_045512.2_21mer_win1_15988 | 15988 | 16008 | 2796 | TGTCTACCATGTGAATACTAA | 3778 | AATCATAAGTGTACCATCTGT |
| NC_045512.2_21mer_win1_15989 | 15989 | 16009 | 2797 | GTCTACCATGTGAATACTAAC | 3779 | CAATCATAAGTGTACCATCTG |
| NC_045512.2_21mer_win1_15990 | 15990 | 16010 | 2798 | TCTACCATGTGAATACTAACT | 3780 | TCAATCATAAGTGTACCATCT |
| NC_045512.2_21mer_win1_16057 | 16057 | 16077 | 2799 | GGATTAGTCCTCATACGACTA | 3781 | ATCAGCATACTCCTGATTAGG |
| NC_045512.2_21mer_win1_16058 | 16058 | 16078 | 2800 | GATTAGTCCTCATACGACTAC | 3782 | CATCAGCATACTCCTGATTAG |
| NC_045512.2_21mer_win1_16059 | 16059 | 16079 | 2801 | ATTAGTCCTCATACGACTACA | 3783 | ACATCAGCATACTCCTGATTA |
| NC_045512.2_21mer_win1_16822 | 16822 | 16842 | 2802 | CCTCTCATGTGGAAACTTTTT | 3784 | TTTTTCAAAGGTGTACTCTCC |
| NC_045512.2_21mer_win1_16823 | 16823 | 16843 | 2803 | CTCTCATGTGGAAACTTTTTC | 3785 | TTTTTCAAAGGTGTACTCTC |
| NC_045512.2_21mer_win1_16824 | 16824 | 16844 | 2804 | TCTCATGTGGAAACTTTTTCC | 3786 | CCTTTTTCAAAGGTGTACTCT |
| NC_045512.2_21mer_win1_16825 | 16825 | 16845 | 2805 | CTCATGTGGAAACTTTTTCCA | 3787 | ACCTTTTTCAAAGGTGTACTC |
| NC_045512.2_21mer_win1_16826 | 16826 | 16846 | 2806 | TCATGTGGAAACTTTTTCCAC | 3788 | CACCTTTTTCAAAGGTGTACT |
| NC_045512.2_21mer_win1_16827 | 16827 | 16847 | 2807 | CATGTGGAAACTTTTTCCACT | 3789 | TCACCTTTTTCAAAGGTGTAC |
| NC_045512.2_21mer_win1_16828 | 16828 | 16848 | 2808 | ATGTGGAAACTTTTTCCACTG | 3790 | GTCACCTTTTTCAAAGGTGTA |
| NC_045512.2_21mer_win1_16829 | 16829 | 16849 | 2809 | TGTGGAAACTTTTTCCACTGA | 3791 | AGTCACCTTTTTCAAAGGTGT |
| NC_045512.2_21mer_win1_16830 | 16830 | 16850 | 2810 | GTGGAAACTTTTTCCACTGAT | 3792 | TAGTCACCTTTTTCAAAGGTG |
| NC_045512.2_21mer_win1_16831 | 16831 | 16851 | 2811 | TGGAAACTTTTTCCACTGATA | 3793 | ATAGTCACCTTTTTCAAAGGT |
| NC_045512.2_21mer_win1_16832 | 16832 | 16852 | 2812 | GGAAACTTTTTCCACTGATAC | 3794 | CATAGTCACCTTTTTCAAAGG |
| NC_045512.2_21mer_win1_16833 | 16833 | 16853 | 2813 | GAAACTTTTTCCACTGATACC | 3795 | CCATAGTCACCTTTTTCAAAG |
| NC_045512.2_21mer_win1_16834 | 16834 | 16854 | 2814 | AAACTTTTTCCACTGATACCA | 3796 | ACCATAGTCACCTTTTTCAAA |
| NC_045512.2_21mer_win1_16835 | 16835 | 16855 | 2815 | AACTTTTTCCACTGATACCAC | 3797 | CACCATAGTCACCTTTTTCAA |
| NC_045512.2_21mer_win1_16836 | 16836 | 16856 | 2816 | ACTTTTTCCACTGATACCACT | 3798 | TCACCATAGTCACCTTTTTCA |
| NC_045512.2_21mer_win1_16837 | 16837 | 16857 | 2817 | CTTTTTCCACTGATACCACTA | 3799 | ATCACCATAGTCACCTTTTTC |
| NC_045512.2_21mer_win1_16838 | 16838 | 16858 | 2818 | TTTTTCCACTGATACCACTAC | 3800 | CATCACCATAGTCACCTTTTT |
| NC_045512.2_21mer_win1_16839 | 16839 | 16859 | 2819 | TTTTCCACTGATACCACTACG | 3801 | GCATCACCATAGTCACCTTTT |
| NC_045512.2_21mer_win1_16840 | 16840 | 16860 | 2820 | TTTCCACTGATACCACTACGA | 3802 | AGCATCACCATAGTCACCTTT |
| NC_045512.2_21mer_win1_16841 | 16841 | 16861 | 2821 | TTCCACTGATACCACTACGAC | 3803 | CAGCATCACCATAGTCACCTT |
| NC_045512.2_21mer_win1_16842 | 16842 | 16862 | 2822 | TCCACTGATACCACTACGACA | 3804 | ACAGCATCACCATAGTCACCT |
| NC_045512.2_21mer_win1_16843 | 16843 | 16863 | 2823 | CCACTGATACCACTACGACAA | 3805 | AACAGCATCACCATAGTCACC |
| NC_045512.2_21mer_win1_16844 | 16844 | 16864 | 2824 | CACTGATACCACTACGACAAC | 3806 | CAACAGCATCACCATAGTCAC |
| NC_045512.2_21mer_win1_16845 | 16845 | 16865 | 2825 | ACTGATACCACTACGACAACA | 3807 | ACAACAGCATCACCATAGTCA |
| NC_045512.2_21mer_win1_16954 | 16954 | 16974 | 2826 | GATCACGGTGTTCTCGTGATA | 3808 | ATAGTGCTCTTGTGGCACTAG |
| NC_045512.2_21mer_win1_16955 | 16955 | 16975 | 2827 | ATCACGGTGTTCTCGTGATAC | 3809 | CATAGTGCTCTTGTGGCACTA |
| NC_045512.2_21mer_win1_16956 | 16956 | 16976 | 2828 | TCACGGTGTTCTCGTGATACA | 3810 | ACATAGTGCTCTTGTGGCACT |
| NC_045512.2_21mer_win1_17008 | 17008 | 17028 | 2829 | TAGAGTCTACTCAAAAGATCG | 3811 | GCTAGAAAACTCATCTGAGAT |
| NC_045512.2_21mer_win1_17009 | 17009 | 17029 | 2830 | AGAGTCTACTCAAAAGATCGT | 3812 | TGCTAGAAAACTCATCTGAGA |
| NC_045512.2_21mer_win1_17010 | 17010 | 17030 | 2831 | GAGTCTACTCAAAAGATCGTT | 3813 | TTGCTAGAAAACTCATCTGAG |
| NC_045512.2_21mer_win1_17011 | 17011 | 17031 | 2832 | AGTCTACTCAAAAGATCGTTA | 3814 | ATTGCTAGAAAACTCATCTGA |
| NC_045512.2_21mer_win1_17012 | 17012 | 17032 | 2833 | GTCTACTCAAAAGATCGTTAC | 3815 | CATTGCTAGAAAACTCATCTG |
| NC_045512.2_21mer_win1_17013 | 17013 | 17033 | 2834 | TCTACTCAAAAGATCGTTACA | 3816 | ACATTGCTAGAAAACTCATCT |
| NC_045512.2_21mer_win1_17014 | 17014 | 17034 | 2835 | CTACTCAAAAGATCGTTACAA | 3817 | AACATTGCTAGAAAACTCATC |
| NC_045512.2_21mer_win1_17015 | 17015 | 17035 | 2836 | TACTCAAAAGATCGTTACAAC | 3818 | CAACATTGCTAGAAAACTCAT |
| NC_045512.2_21mer_win1_17016 | 17016 | 17036 | 2837 | ACTCAAAAGATCGTTACAACG | 3819 | GCAACATTGCTAGAAAACTCA |
| NC_045512.2_21mer_win1_17017 | 17017 | 17037 | 2838 | CTCAAAAGATCGTTACAACGT | 3820 | TGCAACATTGCTAGAAAACTC |
| NC_045512.2_21mer_win1_17018 | 17018 | 17038 | 2839 | TCAAAAGATCGTTACAACGTT | 3821 | TTGCAACATTGCTAGAAAACT |
| NC_045512.2_21mer_win1_17019 | 17019 | 17039 | 2840 | CAAAAGATCGTTACAACGTTT | 3822 | TTTGCAACATTGCTAGAAAAC |
| NC_045512.2_21mer_win1_17020 | 17020 | 17040 | 2841 | AAAAGATCGTTACAACGTTTA | 3823 | ATTTGCAACATTGCTAGAAAA |
| NC_045512.2_21mer_win1_17021 | 17021 | 17041 | 2842 | AAAGATCGTTACAACGTTTAA | 3824 | AATTTGCAACATTGCTAGAAA |
| NC_045512.2_21mer_win1_17022 | 17022 | 17042 | 2843 | AAGATCGTTACAACGTTTAAT | 3825 | TAATTTGCAACATTGCTAGAA |
| NC_045512.2_21mer_win1_17080 | 17080 | 17100 | 2844 | CCTGGTGGACCATGACCATTC | 3826 | CTTACCAGTACCAGGTGGTCC |
| NC_045512.2_21mer_win1_17081 | 17081 | 17101 | 2845 | CTGGTGGACCATGACCATTCT | 3827 | TCTTACCAGTACCAGGTGGTC |
| NC_045512.2_21mer_win1_17082 | 17082 | 17102 | 2846 | TGGTGGACCATGACCATTCTC | 3828 | CTCTTACCAGTACCAGGTGGT |
| NC_045512.2_21mer_win1_17083 | 17083 | 17103 | 2847 | GGTGGACCATGACCATTCTCA | 3829 | ACTCTTACCAGTACCAGGTGG |
| NC_045512.2_21mer_win1_17084 | 17084 | 17104 | 2848 | GTGGACCATGACCATTCTCAG | 3830 | GACTCTTACCAGTACCAGGTG |
| NC_045512.2_21mer_win1_17085 | 17085 | 17105 | 2849 | TGGACCATGACCATTCTCAGT | 3831 | TGACTCTTACCAGTACCAGGT |
| NC_045512.2_21mer_win1_17086 | 17086 | 17106 | 2850 | GGACCATGACCATTCTCAGTA | 3832 | ATGACTCTTACCAGTACCAGG |
| NC_045512.2_21mer_win1_17087 | 17087 | 17107 | 2851 | GACCATGACCATTCTCAGTAA | 3833 | AATGACTCTTACCAGTACCAG |
| NC_045512.2_21mer_win1_17088 | 17088 | 17108 | 2852 | ACCATGACCATTCTCAGTAAA | 3834 | AAATGACTCTTACCAGTACCA |
| NC_045512.2_21mer_win1_17089 | 17089 | 17109 | 2853 | CCATGACCATTCTCAGTAAAA | 3835 | AAAATGACTCTTACCAGTACC |
| NC_045512.2_21mer_win1_17090 | 17090 | 17110 | 2854 | CATGACCATTCTCAGTAAAAC | 3836 | CAAAATGACTCTTACCAGTAC |
| NC_045512.2_21mer_win1_17091 | 17091 | 17111 | 2855 | ATGACCATTCTCAGTAAAACG | 3837 | GCAAAATGACTCTTACCAGTA |
| NC_045512.2_21mer_win1_17269 | 17269 | 17289 | 2856 | TTTAAGTTTCACTTAAGTTGT | 3838 | TGTTGAATTCACTTTGAATTT |
| NC_045512.2_21mer_win1_18100 | 18100 | 18120 | 2857 | TGTGTCCGTGGATGTGTGGAG | 3839 | GAGGTGTGTAGGTGCCTGTGT |
| NC_045512.2_21mer_win1_18101 | 18101 | 18121 | 2858 | GTGTCCGTGGATGTGTGGAGT | 3840 | TGAGGTGTGTAGGTGCCTGTG |
| NC_045512.2_21mer_win1_18102 | 18102 | 18122 | 2859 | TGTCCGTGGATGTGTGGAGTC | 3841 | CTGAGGTGTGTAGGTGCCTGT |
| NC_045512.2_21mer_win1_18196 | 18196 | 18216 | 2860 | TCTGAGTAGAGATACTACCCA | 3842 | ACCCATCATAGAGATGAGTCT |
| NC_045512.2_21mer_win1_18197 | 18197 | 18217 | 2861 | CTGAGTAGAGATACTACCCAA | 3843 | AACCCATCATAGAGATGAGTC |
| NC_045512.2_21mer_win1_18198 | 18198 | 18218 | 2862 | TGAGTAGAGATACTACCCAAA | 3844 | AAACCCATCATAGAGATGAGT |
| NC_045512.2_21mer_win1_19618 | 19618 | 19638 | 2863 | GTCTCAAATCTTTTACACCGA | 3845 | AGCCACATTTTCTAAACTCTG |
| NC_045512.2_21mer_win1_19619 | 19619 | 19639 | 2864 | TCTCAAATCTTTTACACCGAA | 3846 | AAGCCACATTTTCTAAACTCT |
| NC_045512.2_21mer_win1_20107 | 20107 | 20127 | 2865 | TTACCTCAGTGTAATTAACCT | 3847 | TCCAATTAATGTGACTCCATT |
| NC_045512.2_21mer_win1_20108 | 20108 | 20128 | 2866 | TACCTCAGTGTAATTAACCTC | 3848 | CTCCAATTAATGTGACTCCAT |
| NC_045512.2_21mer_win1_20109 | 20109 | 20129 | 2867 | ACCTCAGTGTAATTAACCTCT | 3849 | TCTCCAATTAATGTGACTCCA |
| NC_045512.2_21mer_win1_20110 | 20110 | 20130 | 2868 | CCTCAGTGTAATTAACCTCTT | 3850 | TTCTCCAATTAATGTGACTCC |
| NC_045512.2_21mer_win1_21502 | 21502 | 21522 | 2869 | TAATCTCTTTTGTTGTCTCAA | 3851 | AACTCTGTTGTTTTCTCTAAT |
| NC_045512.2_21mer_win1_21503 | 21503 | 21523 | 2870 | AATCTCTTTTGTTGTCTCAAC | 3852 | CAACTCTGTTGTTTTCTCTAA |
| NC_045512.2_21mer_win1_21504 | 21504 | 21524 | 2871 | ATCTCTTTTGTTGTCTCAACA | 3853 | ACAACTCTGTTGTTTTCTCTA |
| NC_045512.2_21mer_win1_24302 | 24302 | 24322 | 2872 | TTACAAGAGATACTCTTGGTT | 3854 | TTGGTTCTCATAGAGAACATT |
| NC_045512.2_21mer_win1_24303 | 24303 | 24323 | 2873 | TACAAGAGATACTCTTGGTTT | 3855 | TTTGGTTCTCATAGAGAACAT |
| NC_045512.2_21mer_win1_24304 | 24304 | 24324 | 2874 | ACAAGAGATACTCTTGGTTTT | 3856 | TTTTGGTTCTCATAGAGAACA |
| NC_045512.2_21mer_win1_24305 | 24305 | 24325 | 2875 | CAAGAGATACTCTTGGTTTTT | 3857 | TTTTTGGTTCTCATAGAGAAC |
| NC_045512.2_21mer_win1_24620 | 24620 | 24640 | 2876 | CGAAGACGATTAGAACGACGA | 3858 | AGCAGCAAGATTAGCAGAAGC |
| NC_045512.2_21mer_win1_24621 | 24621 | 24641 | 2877 | GAAGACGATTAGAACGACGAT | 3859 | TAGCAGCAAGATTAGCAGAAG |
| NC_045512.2_21mer_win1_24622 | 24622 | 24642 | 2878 | AAGACGATTAGAACGACGATG | 3860 | GTAGCAGCAAGATTAGCAGAA |
| NC_045512.2_21mer_win1_24623 | 24623 | 24643 | 2879 | AGACGATTAGAACGACGATGA | 3861 | AGTAGCAGCAAGATTAGCAGA |
| NC_045512.2_21mer_win1_24624 | 24624 | 24644 | 2880 | GACGATTAGAACGACGATGAT | 3862 | TAGTAGCAGCAAGATTAGCAG |
| NC_045512.2_21mer_win1_24625 | 24625 | 24645 | 2881 | ACGATTAGAACGACGATGATT | 3863 | TTAGTAGCAGCAAGATTAGCA |
| NC_045512.2_21mer_win1_24626 | 24626 | 24646 | 2882 | CGATTAGAACGACGATGATTT | 3864 | TTTAGTAGCAGCAAGATTAGC |
| NC_045512.2_21mer_win1_24627 | 24627 | 24647 | 2883 | GATTAGAACGACGATGATTTT | 3865 | TTTTAGTAGCAGCAAGATTAG |
| NC_045512.2_21mer_win1_24628 | 24628 | 24648 | 2884 | ATTAGAACGACGATGATTTTA | 3866 | ATTTTAGTAGCAGCAAGATTA |
| NC_045512.2_21mer_win1_24629 | 24629 | 24649 | 2885 | TTAGAACGACGATGATTTTAC | 3867 | CATTTTAGTAGCAGCAAGATT |
| NC_045512.2_21mer_win1_24630 | 24630 | 24650 | 2886 | TAGAACGACGATGATTTTACA | 3868 | ACATTTTAGTAGCAGCAAGAT |
| NC_045512.2_21mer_win1_24631 | 24631 | 24651 | 2887 | AGAACGACGATGATTTTACAG | 3869 | GACATTTTAGTAGCAGCAAGA |
| NC_045512.2_21mer_win1_24662 | 24662 | 24682 | 2888 | GAACCTGTTAGTTTTTCTCAA | 3870 | AACTCTTTTTGATTGTCCAAG |
| NC_045512.2_21mer_win1_24663 | 24663 | 24683 | 2889 | AACCTGTTAGTTTTTCTCAAC | 3871 | CAACTCTTTTTGATTGTCCAA |
| NC_045512.2_21mer_win1_24664 | 24664 | 24684 | 2890 | ACCTGTTAGTTTTTCTCAACT | 3872 | TCAACTCTTTTTGATTGTCCA |
| NC_045512.2_21mer_win1_25034 | 25034 | 25054 | 2891 | TTAGTATGTAGTGGTCTACAA | 3873 | AACATCTGGTGATGTATGATT |
| NC_045512.2_2lmer_win1_25035 | 25035 | 25055 | 2892 | TAGTATGTAGTGGTCTACAAC | 3874 | CAACATCTGGTGATGTATGAT |
| NC_045512.2_21mer_win1_25036 | 25036 | 25056 | 2893 | AGTATGTAGTGGTCTACAACT | 3875 | TCAACATCTGGTGATGTATGA |
| NC_045512.2_21mer_win1_25037 | 25037 | 25057 | 2894 | GTATGTAGTGGTCTACAACTA | 3876 | ATCAACATCTGGTGATGTATG |
| NC_045512.2_2lmer_win1_25104 | 25104 | 25124 | 2895 | TTCTTTAACTGGCGGAGTTAC | 3877 | CATTGAGGCGGTCAATTTCTT |
| NC_045512.2_2lmer_win1_25105 | 25105 | 25125 | 2896 | TCTTTAACTGGCGGAGTTACT | 3878 | TCATTGAGGCGGTCAATTTCT |
| NC_045512.2_2lmer_win1_25106 | 25106 | 25126 | 2897 | CTTTAACTGGCGGAGTTACTC | 3879 | CTCATTGAGGCGGTCAATTTC |
| NC_045512.2_2lmer_win1_25107 | 25107 | 25127 | 2898 | TTTAACTGGCGGAGTTACTCC | 3880 | CCTCATTGAGGCGGTCAATTT |
| NC_045512.2_2lmer_win1_25108 | 25108 | 25128 | 2899 | TTAACTGGCGGAGTTACTCCA | 3881 | ACCTCATTGAGGCGGTCAATT |
| NC_045512.2_21mer_win1_25364 | 25364 | 25384 | 2900 | CAGTTTAATGTAATGTGTATT | 3882 | TTATGTGTAATGTAATTTGAC |
| NC_045512.2_21mer_win1_25365 | 25365 | 25385 | 2901 | AGTTTAATGTAATGTGTATTT | 3883 | TTTATGTGTAATGTAATTTGA |
| NC_045512.2_21mer_win1_25366 | 25366 | 25386 | 2902 | GTTTAATGTAATGTGTATTTG | 3884 | GTTTATGTGTAATGTAATTTG |
| NC_045512.2_21mer_win1_25367 | 25367 | 25387 | 2903 | TTTAATGTAATGTGTATTTGC | 3885 | CGTTTATGTGTAATGTAATTT |
| NC_045512.2_21mer_win1_25502 | 25502 | 25522 | 2904 | ATGTTCGGAGTGAGGGAAAGC | 3886 | CGAAAGGGAGTGAGGCTTGTA |
| NC_045512.2_21mer_win1_25503 | 25503 | 25523 | 2905 | TGTTCGGAGTGAGGGAAAGCC | 3887 | CCGAAAGGGAGTGAGGCTTGT |
| NC_045512.2_21mer_win1_25504 | 25504 | 25524 | 2906 | GTTCGGAGTGAGGGAAAGCCT | 3888 | TCCGAAAGGGAGTGAGGCTTG |
| NC_045512.2_21mer_win1_25505 | 25505 | 25525 | 2907 | TTCGGAGTGAGGGAAAGCCTA | 3889 | ATCCGAAAGGGAGTGAGGCTT |
| NC_045512.2_21mer_win1_25506 | 25506 | 25526 | 2908 | TCGGAGTGAGGGAAAGCCTAC | 3890 | CATCCGAAAGGGAGTGAGGCT |
| NC_045512.2_21mer_win1_25507 | 25507 | 25527 | 2909 | CGGAGTGAGGGAAAGCCTACC | 3891 | CCATCCGAAAGGGAGTGAGGC |
| NC_045512.2_21mer_win1_25508 | 25508 | 25528 | 2910 | GGAGTGAGGGAAAGCCTACCG | 3892 | GCCATCCGAAAGGGAGTGAGG |
| NC_045512.2_21mer_win1_25509 | 25509 | 25529 | 2911 | GAGTGAGGGAAAGCCTACCGA | 3893 | AGCCATCCGAAAGGGAGTGAG |
| NC_045512.2_21mer_win1_25510 | 25510 | 25530 | 2912 | AGTGAGGGAAAGCCTACCGAA | 3894 | AAGCCATCCGAAAGGGAGTGA |
| NC_045512.2_21mer_win1_26191 | 26191 | 26211 | 2913 | GGCTGCTGCTGATGATCGCAC | 3895 | CACGCTAGTAGTCGTCGTCGG |
| NC_045512.2_21mer_win1_26192 | 26192 | 26212 | 2914 | GCTGCTGCTGATGATCGCACG | 3896 | GCACGCTAGTAGTCGTCGTCG |
| NC_045512.2_21mer_win1_26193 | 26193 | 26213 | 2915 | CTGCTGCTGATGATCGCACGG | 3897 | GGCACGCTAGTAGTCGTCGTC |
| NC_045512.2_21mer_win1_26194 | 26194 | 26214 | 2916 | TGCTGCTGATGATCGCACGGA | 3898 | AGGCACGCTAGTAGTCGTCGT |
| NC_045512.2_21mer_win1_26195 | 26195 | 26215 | 2917 | GCTGCTGATGATCGCACGGAA | 3899 | AAGGCACGCTAGTAGTCGTCG |
| NC_045512.2_21mer_win1_26196 | 26196 | 26216 | 2918 | CTGCTGATGATCGCACGGAAA | 3900 | AAAGGCACGCTAGTAGTCGTC |
| NC_045512.2_21mer_win1_26197 | 26197 | 26217 | 2919 | TGCTGATGATCGCACGGAAAC | 3901 | CAAAGGCACGCTAGTAGTCGT |
| NC_045512.2_21mer_win1_26198 | 26198 | 26218 | 2920 | GCTGATGATCGCACGGAAACA | 3902 | ACAAAGGCACGCTAGTAGTCG |
| NC_045512.2_21mer_win1_26199 | 26199 | 26219 | 2921 | CTGATGATCGCACGGAAACAT | 3903 | TACAAAGGCACGCTAGTAGTC |
| NC_045512.2_21mer_win1_26200 | 26200 | 26220 | 2922 | TGATGATCGCACGGAAACATT | 3904 | TTACAAAGGCACGCTAGTAGT |
| NC_045512.2_21mer_win1_26201 | 26201 | 26221 | 2923 | GATGATCGCACGGAAACATTC | 3905 | CTTACAAAGGCACGCTAGTAG |
| NC_045512.2_21mer_win1_26202 | 26202 | 26222 | 2924 | ATGATCGCACGGAAACATTCG | 3906 | GCTTACAAAGGCACGCTAGTA |
| NC_045512.2_21mer_win1_26203 | 26203 | 26223 | 2925 | TGATCGCACGGAAACATTCGT | 3907 | TGCTTACAAAGGCACGCTAGT |
| NC_045512.2_21mer_win1_26204 | 26204 | 26224 | 2926 | GATCGCACGGAAACATTCGTG | 3908 | GTGCTTACAAAGGCACGCTAG |
| NC_045512.2_21mer_win1_26205 | 26205 | 26225 | 2927 | ATCGCACGGAAACATTCGTGT | 3909 | TGTGCTTACAAAGGCACGCTA |
| NC_045512.2_21mer_win1_26206 | 26206 | 26226 | 2928 | TCGCACGGAAACATTCGTGTT | 3910 | TTGTGCTTACAAAGGCACGCT |
| NC_045512.2_21mer_win1_26207 | 26207 | 26227 | 2929 | CGCACGGAAACATTCGTGTTC | 3911 | CTTGTGCTTACAAAGGCACGC |
| NC_045512.2_21mer_win1_26232 | 26232 | 26252 | 2930 | ACTCATGCTTGAATACATGAG | 3912 | GAGTACATAAGTTCGTACTCA |
| NC_045512.2_21mer_win1_26233 | 26233 | 26253 | 2931 | CTCATGCTTGAATACATGAGT | 3913 | TGAGTACATAAGTTCGTACTC |
| NC_045512.2_21mer_win1_26234 | 26234 | 26254 | 2932 | TCATGCTTGAATACATGAGTA | 3914 | ATGAGTACATAAGTTCGTACT |
| NC_045512.2_21mer_win1_26235 | 26235 | 26255 | 2933 | CATGCTTGAATACATGAGTAA | 3915 | AATGAGTACATAAGTTCGTAC |
| NC_045512.2_21mer_win1_26236 | 26236 | 26256 | 2934 | ATGCTTGAATACATGAGTAAG | 3916 | GAATGAGTACATAAGTTCGTA |
| NC_045512.2_21mer_win1_26237 | 26237 | 26257 | 2935 | TGCTTGAATACATGAGTAAGC | 3917 | CGAATGAGTACATAAGTTCGT |
| NC_045512.2_21mer_win1_26238 | 26238 | 26258 | 2936 | GCTTGAATACATGAGTAAGCA | 3918 | ACGAATGAGTACATAAGTTCG |
| NC_045512.2_21mer_win1_26239 | 26239 | 26259 | 2937 | CTTGAATACATGAGTAAGCAA | 3919 | AACGAATGAGTACATAAGTTC |
| NC_045512.2_21mer_win1_26240 | 26240 | 26260 | 2938 | TTGAATACATGAGTAAGCAAA | 3920 | AAACGAATGAGTACATAAGTT |
| NC_045512.2_21mer_win1_26241 | 26241 | 26261 | 2939 | TGAATACATGAGTAAGCAAAG | 3921 | GAAACGAATGAGTACATAAGT |
| NC_045512.2_21mer_win1_26242 | 26242 | 26262 | 2940 | GAATACATGAGTAAGCAAAGC | 3922 | CGAAACGAATGAGTACATAAG |
| NC_045512.2_21mer_win1_26243 | 26243 | 26263 | 2941 | AATACATGAGTAAGCAAAGCC | 3923 | CCGAAACGAATGAGTACATAA |
| NC_045512.2_21mer_win1_26244 | 26244 | 26264 | 2942 | ATACATGAGTAAGCAAAGCCT | 3924 | TCCGAAACGAATGAGTACATA |
| NC_045512.2_21mer_win1_26245 | 26245 | 26265 | 2943 | TACATGAGTAAGCAAAGCCTT | 3925 | TTCCGAAACGAATGAGTACAT |
| NC_045512.2_21mer_win1_26246 | 26246 | 26266 | 2944 | ACATGAGTAAGCAAAGCCTTC | 3926 | CTTCCGAAACGAATGAGTACA |
| NC_045512.2_21mer_win1_26247 | 26247 | 26267 | 2945 | CATGAGTAAGCAAAGCCTTCT | 3927 | TCTTCCGAAACGAATGAGTAC |
| NC_045512.2_21mer_win1_26269 | 26269 | 26289 | 2946 | TGTCCATGCAATTATCAATTA | 3928 | ATTAACTATTAACGTACCTGT |
| NC_045512.2_21mer_win1_26270 | 26270 | 26290 | 2947 | GTCCATGCAATTATCAATTAT | 3929 | TATTAACTATTAACGTACCTG |
| NC_045512.2_21mer_win1_26271 | 26271 | 26291 | 2948 | TCCATGCAATTATCAATTATC | 3930 | CTATTAACTATTAACGTACCT |
| NC_045512.2_21mer_win1_26272 | 26272 | 26292 | 2949 | CCATGCAATTATCAATTATCG | 3931 | GCTATTAACTATTAACGTACC |
| NC_045512.2_21mer_win1_26273 | 26273 | 26293 | 2950 | CATGCAATTATCAATTATCGC | 3932 | CGCTATTAACTATTAACGTAC |
| NC_045512.2_21mer_win1_26274 | 26274 | 26294 | 2951 | ATGCAATTATCAATTATCGCA | 3933 | ACGCTATTAACTATTAACGTA |
| NC_045512.2_21mer_win1_26275 | 26275 | 26295 | 2952 | TGCAATTATCAATTATCGCAT | 3934 | TACGCTATTAACTATTAACGT |
| NC_045512.2_21mer_win1_26276 | 26276 | 26296 | 2953 | GCAATTATCAATTATCGCATG | 3935 | GTACGCTATTAACTATTAACG |
| NC_045512.2_21mer_win1_26277 | 26277 | 26297 | 2954 | CAATTATCAATTATCGCATGA | 3936 | AGTACGCTATTAACTATTAAC |
| NC_045512.2_21mer_win1_26278 | 26278 | 26298 | 2955 | AATTATCAATTATCGCATGAA | 3937 | AAGTACGCTATTAACTATTAA |
| NC_045512.2_21mer_win1_26279 | 26279 | 26299 | 2956 | ATTATCAATTATCGCATGAAG | 3938 | GAAGTACGCTATTAACTATTA |
| NC_045512.2_21mer_win1_26280 | 26280 | 26300 | 2957 | TTATCAATTATCGCATGAAGA | 3939 | AGAAGTACGCTATTAACTATT |
| NC_045512.2_21mer_win1_26281 | 26281 | 26301 | 2958 | TATCAATTATCGCATGAAGAA | 3940 | AAGAAGTACGCTATTAACTAT |
| NC_045512.2_21mer_win1_26282 | 26282 | 26302 | 2959 | ATCAATTATCGCATGAAGAAA | 3941 | AAAGAAGTACGCTATTAACTA |
| NC_045512.2_21mer_win1_26283 | 26283 | 26303 | 2960 | TCAATTATCGCATGAAGAAAA | 3942 | AAAAGAAGTACGCTATTAACT |
| NC_045512.2_21mer_win1_26284 | 26284 | 26304 | 2961 | CAATTATCGCATGAAGAAAAA | 3943 | AAAAAGAAGTACGCTATTAAC |
| NC_045512.2_21mer_win1_26285 | 26285 | 26305 | 2962 | AATTATCGCATGAAGAAAAAG | 3944 | GAAAAAGAAGTACGCTATTAA |
| NC_045512.2_21mer_win1_26286 | 26286 | 26306 | 2963 | ATTATCGCATGAAGAAAAAGA | 3945 | AGAAAAAGAAGTACGCTATTA |
| NC_045512.2_21mer_win1_26287 | 26287 | 26307 | 2964 | TTATCGCATGAAGAAAAAGAA | 3946 | AAGAAAAAGAAGTACGCTATT |
| NC_045512.2_21mer_win1_26288 | 26288 | 26308 | 2965 | TATCGCATGAAGAAAAAGAAC | 3947 | CAAGAAAAAGAAGTACGCTAT |
| NC_045512.2_21mer_win1_26289 | 26289 | 26309 | 2966 | ATCGCATGAAGAAAAAGAACG | 3948 | GCAAGAAAAAGAAGTACGCTA |
| NC_045512.2_21mer_win1_26290 | 26290 | 26310 | 2967 | TCGCATGAAGAAAAAGAACGA | 3949 | AGCAAGAAAAAGAAGTACGCT |
| NC_045512.2_21mer_win1_26291 | 26291 | 26311 | 2968 | CGCATGAAGAAAAAGAACGAA | 3950 | AAGCAAGAAAAAGAAGTACGC |
| NC_045512.2_21mer_win1_26292 | 26292 | 26312 | 2969 | GCATGAAGAAAAAGAACGAAA | 3951 | AAAGCAAGAAAAAGAAGTACG |
| NC_045512.2_21mer_win1_26293 | 26293 | 26313 | 2970 | CATGAAGAAAAAGAACGAAAG | 3952 | GAAAGCAAGAAAAAGAAGTAC |
| NC_045512.2_21mer_win1_26294 | 26294 | 26314 | 2971 | ATGAAGAAAAAGAACGAAAGC | 3953 | CGAAAGCAAGAAAAAGAAGTA |
| NC_045512.2_21mer_win1_26295 | 26295 | 26315 | 2972 | TGAAGAAAAAGAACGAAAGCA | 3954 | ACGAAAGCAAGAAAAAGAAGT |
| NC_045512.2_21mer_win1_26296 | 26296 | 26316 | 2973 | GAAGAAAAAGAACGAAAGCAC | 3955 | CACGAAAGCAAGAAAAAGAAG |
| NC_045512.2_21mer_win1_26297 | 26297 | 26317 | 2974 | AAGAAAAAGAACGAAAGCACC | 3956 | CCACGAAAGCAAGAAAAAGAA |
| NC_045512.2_21mer_win1_26298 | 26298 | 26318 | 2975 | AGAAAAAGAACGAAAGCACCA | 3957 | ACCACGAAAGCAAGAAAAAGA |
| NC_045512.2_21mer_win1_26299 | 26299 | 26319 | 2976 | GAAAAAGAACGAAAGCACCAT | 3958 | TACCACGAAAGCAAGAAAAAG |
| NC_045512.2_21mer_win1_26300 | 26300 | 26320 | 2977 | AAAAAGAACGAAAGCACCATA | 3959 | ATACCACGAAAGCAAGAAAAA |
| NC_045512.2_21mer_win1_26301 | 26301 | 26321 | 2978 | AAAAGAACGAAAGCACCATAA | 3960 | AATACCACGAAAGCAAGAAAA |
| NC_045512.2_21mer_win1_26302 | 26302 | 26322 | 2979 | AAAGAACGAAAGCACCATAAG | 3961 | GAATACCACGAAAGCAAGAAA |
| NC_045512.2_21mer_win1_26303 | 26303 | 26323 | 2980 | AAGAACGAAAGCACCATAAGA | 3962 | AGAATACCACGAAAGCAAGAA |
| NC_045512.2_21mer_win1_26304 | 26304 | 26324 | 2981 | AGAACGAAAGCACCATAAGAA | 3963 | AAGAATACCACGAAAGCAAGA |
| NC_045512.2_21mer_win1_26305 | 26305 | 26325 | 2982 | GAACGAAAGCACCATAAGAAC | 3964 | CAAGAATACCACGAAAGCAAG |
| NC_045512.2_21mer_win1_26306 | 26306 | 26326 | 2983 | AACGAAAGCACCATAAGAACG | 3965 | GCAAGAATACCACGAAAGCAA |
| NC_045512.2_21mer_win1_26307 | 26307 | 26327 | 2984 | ACGAAAGCACCATAAGAACGA | 3966 | AGCAAGAATACCACGAAAGCA |
| NC_045512.2_21mer_win1_26308 | 26308 | 26328 | 2985 | CGAAAGCACCATAAGAACGAT | 3967 | TAGCAAGAATACCACGAAAGC |
| NC_045512.2_21mer_win1_26309 | 26309 | 26329 | 2986 | GAAAGCACCATAAGAACGATC | 3968 | CTAGCAAGAATACCACGAAAG |
| NC_045512.2_21mer_win1_26310 | 26310 | 26330 | 2987 | AAAGCACCATAAGAACGATCA | 3969 | ACTAGCAAGAATACCACGAAA |
| NC_045512.2_21mer_win1_26332 | 26332 | 26352 | 2988 | TGTGATCGGTAGGAATGACGC | 3970 | CGCAGTAAGGATGGCTAGTGT |
| NC_045512.2_21mer_win1_26333 | 26333 | 26353 | 2989 | GTGATCGGTAGGAATGACGCG | 3971 | GCGCAGTAAGGATGGCTAGTG |
| NC_045512.2_21mer_win1_26334 | 26334 | 26354 | 2990 | TGATCGGTAGGAATGACGCGA | 3972 | AGCGCAGTAAGGATGGCTAGT |
| NC_045512.2_21mer_win1_26335 | 26335 | 26355 | 2991 | GATCGGTAGGAATGACGCGAA | 3973 | AAGCGCAGTAAGGATGGCTAG |
| NC_045512.2_21mer_win1_26336 | 26336 | 26356 | 2992 | ATCGGTAGGAATGACGCGAAG | 3974 | GAAGCGCAGTAAGGATGGCTA |
| NC_045512.2_21mer_win1_26337 | 26337 | 26357 | 2993 | TCGGTAGGAATGACGCGAAGC | 3975 | CGAAGCGCAGTAAGGATGGCT |
| NC_045512.2_21mer_win1_26338 | 26338 | 26358 | 2994 | CGGTAGGAATGACGCGAAGCT | 3976 | TCGAAGCGCAGTAAGGATGGC |
| NC_045512.2_21mer_win1_26339 | 26339 | 26359 | 2995 | GGTAGGAATGACGCGAAGCTA | 3977 | ATCGAAGCGCAGTAAGGATGG |
| NC_045512.2_21mer_win1_26340 | 26340 | 26360 | 2996 | GTAGGAATGACGCGAAGCTAA | 3978 | AATCGAAGCGCAGTAAGGATG |
| NC_045512.2_21mer_win1_26341 | 26341 | 26361 | 2997 | TAGGAATGACGCGAAGCTAAC | 3979 | CAATCGAAGCGCAGTAAGGAT |
| NC_045512.2_21mer_win1_26342 | 26342 | 26362 | 2998 | AGGAATGACGCGAAGCTAACA | 3980 | ACAATCGAAGCGCAGTAAGGA |
| NC_045512.2_21mer_win1_26343 | 26343 | 26363 | 2999 | GGAATGACGCGAAGCTAACAC | 3981 | CACAATCGAAGCGCAGTAAGG |
| NC_045512.2_21mer_win1_26344 | 26344 | 26364 | 3000 | GAATGACGCGAAGCTAACACA | 3982 | ACACAATCGAAGCGCAGTAAG |
| NC_045512.2_21mer_win1_26345 | 26345 | 26365 | 3001 | AATGACGCGAAGCTAACACAC | 3983 | CACACAATCGAAGCGCAGTAA |
| NC_045512.2_21mer_win1_26346 | 26346 | 26366 | 3002 | ATGACGCGAAGCTAACACACG | 3984 | GCACACAATCGAAGCGCAGTA |
| NC_045512.2_21mer_win1_26347 | 26347 | 26367 | 3003 | TGACGCGAAGCTAACACACGC | 3985 | CGCACACAATCGAAGCGCAGT |
| NC_045512.2_21mer_win1_26348 | 26348 | 26368 | 3004 | GACGCGAAGCTAACACACGCA | 3986 | ACGCACACAATCGAAGCGCAG |
| NC_045512.2_21mer_win1_26349 | 26349 | 26369 | 3005 | ACGCGAAGCTAACACACGCAT | 3987 | TACGCACACAATCGAAGCGCA |
| NC_045512.2_21mer_win1_26350 | 26350 | 26370 | 3006 | CGCGAAGCTAACACACGCATG | 3988 | GTACGCACACAATCGAAGCGC |
| NC_045512.2_21mer_win1_26351 | 26351 | 26371 | 3007 | GCGAAGCTAACACACGCATGA | 3989 | AGTACGCACACAATCGAAGCG |
| NC_045512.2_21mer_win1_26352 | 26352 | 26372 | 3008 | CGAAGCTAACACACGCATGAC | 3990 | CAGTACGCACACAATCGAAGC |
| NC_045512.2_21mer_win1_26353 | 26353 | 26373 | 3009 | GAAGCTAACACACGCATGACG | 3991 | GCAGTACGCACACAATCGAAG |
| NC_045512.2_21mer_win1_26354 | 26354 | 26374 | 3010 | AAGCTAACACACGCATGACGA | 3992 | AGCAGTACGCACACAATCGAA |
| NC_045512.2_21mer_win1_26355 | 26355 | 26375 | 3011 | AGCTAACACACGCATGACGAC | 3993 | CAGCAGTACGCACACAATCGA |
| NC_045512.2_21mer_win1_26356 | 26356 | 26376 | 3012 | GCTAACACACGCATGACGACG | 3994 | GCAGCAGTACGCACACAATCG |
| NC_045512.2_21mer_win1_26357 | 26357 | 26377 | 3013 | CTAACACACGCATGACGACGT | 3995 | TGCAGCAGTACGCACACAATC |
| NC_045512.2_21mer_win1_26358 | 26358 | 26378 | 3014 | TAACACACGCATGACGACGTT | 3996 | TTGCAGCAGTACGCACACAAT |
| NC_045512.2_21mer_win1_26359 | 26359 | 26379 | 3015 | AACACACGCATGACGACGTTA | 3997 | ATTGCAGCAGTACGCACACAA |
| NC_045512.2_21mer_win1_26360 | 26360 | 26380 | 3016 | ACACACGCATGACGACGTTAT | 3998 | TATTGCAGCAGTACGCACACA |
| NC_045512.2_21mer_win1_26361 | 26361 | 26381 | 3017 | CACACGCATGACGACGTTATA | 3999 | ATATTGCAGCAGTACGCACAC |
| NC_045512.2_21mer_win1_26362 | 26362 | 26382 | 3018 | ACACGCATGACGACGTTATAA | 4000 | AATATTGCAGCAGTACGCACA |
| NC_045512.2_21mer_win1_26363 | 26363 | 26383 | 3019 | CACGCATGACGACGTTATAAC | 4001 | CAATATTGCAGCAGTACGCAC |
| NC_045512.2_21mer_win1_26364 | 26364 | 26384 | 3020 | ACGCATGACGACGTTATAACA | 4002 | ACAATATTGCAGCAGTACGCA |
| NC_045512.2_21mer_win1_26365 | 26365 | 26385 | 3021 | CGCATGACGACGTTATAACAA | 4003 | AACAATATTGCAGCAGTACGC |
| NC_045512.2_21mer_win1_26366 | 26366 | 26386 | 3022 | GCATGACGACGTTATAACAAT | 4004 | TAACAATATTGCAGCAGTACG |
| NC_045512.2_21mer_win1_26367 | 26367 | 26387 | 3023 | CATGACGACGTTATAACAATT | 4005 | TTAACAATATTGCAGCAGTAC |
| NC_045512.2_21mer_win1_26368 | 26368 | 26388 | 3024 | ATGACGACGTTATAACAATTG | 4006 | GTTAACAATATTGCAGCAGTA |
| NC_045512.2_21mer_win1_26369 | 26369 | 26389 | 3025 | TGACGACGTTATAACAATTGC | 4007 | CGTTAACAATATTGCAGCAGT |
| NC_045512.2_21mer_win1_26370 | 26370 | 26390 | 3026 | GACGACGTTATAACAATTGCA | 4008 | ACGTTAACAATATTGCAGCAG |
| NC_045512.2_21mer_win1_26371 | 26371 | 26391 | 3027 | ACGACGTTATAACAATTGCAC | 4009 | CACGTTAACAATATTGCAGCA |
| NC_045512.2_21mer_win1_26372 | 26372 | 26392 | 3028 | CGACGTTATAACAATTGCACT | 4010 | TCACGTTAACAATATTGCAGC |
| NC_045512.2_21mer_win1_26373 | 26373 | 26393 | 3029 | GACGTTATAACAATTGCACTC | 4011 | CTCACGTTAACAATATTGCAG |
| NC_045512.2_21mer_win1_26374 | 26374 | 26394 | 3030 | ACGTTATAACAATTGCACTCA | 4012 | ACTCACGTTAACAATATTGCA |
| NC_045512.2_21mer_win1_26450 | 26450 | 26470 | 3031 | CTCAAGGACTAGAAGACCAGA | 4013 | AGACCAGAAGATCAGGAACTC |
| NC_045512.2_21mer_win1_26451 | 26451 | 26471 | 3032 | TCAAGGACTAGAAGACCAGAT | 4014 | TAGACCAGAAGATCAGGAACT |
| NC_045512.2_21mer_win1_26452 | 26452 | 26472 | 3033 | CAAGGACTAGAAGACCAGATT | 4015 | TTAGACCAGAAGATCAGGAAC |
| NC_045512.2_21mer_win1_26453 | 26453 | 26473 | 3034 | AAGGACTAGAAGACCAGATTT | 4016 | TTTAGACCAGAAGATCAGGAA |
| NC_045512.2_21mer_win1_26454 | 26454 | 26474 | 3035 | AGGACTAGAAGACCAGATTTG | 4017 | GTTTAGACCAGAAGATCAGGA |
| NC_045512.2_21mer_win1_26455 | 26455 | 26475 | 3036 | GGACTAGAAGACCAGATTTGC | 4018 | CGTTTAGACCAGAAGATCAGG |
| NC_045512.2_21mer_win1_26456 | 26456 | 26476 | 3037 | GACTAGAAGACCAGATTTGCT | 4019 | TCGTTTAGACCAGAAGATCAG |
| NC_045512.2_21mer_win1_26457 | 26457 | 26477 | 3038 | ACTAGAAGACCAGATTTGCTT | 4020 | TTCGTTTAGACCAGAAGATCA |
| NC_045512.2_21mer_win1_26458 | 26458 | 26478 | 3039 | CTAGAAGACCAGATTTGCTTG | 4021 | GTTCGTTTAGACCAGAAGATC |
| NC_045512.2_21mer_win1_26459 | 26459 | 26479 | 3040 | TAGAAGACCAGATTTGCTTGA | 4022 | AGTTCGTTTAGACCAGAAGAT |
| NC_045512.2_21mer_win1_26460 | 26460 | 26480 | 3041 | AGAAGACCAGATTTGCTTGAT | 4023 | TAGTTCGTTTAGACCAGAAGA |
| NC_045512.2_21mer_win1_26461 | 26461 | 26481 | 3042 | GAAGACCAGATTTGCTTGATT | 4024 | TTAGTTCGTTTAGACCAGAAG |
| NC_045512.2_21mer_win1_26574 | 26574 | 26594 | 3043 | CTTGTTACCTTGGATCATTAT | 4025 | TATTACTAGGTTCCATTGTTC |
| NC_045512.2_21mer_win1_26575 | 26575 | 26595 | 3044 | TTGTTACCTTGGATCATTATC | 4026 | CTATTACTAGGTTCCATTGTT |
| NC_045512.2_21mer_win1_26576 | 26576 | 26596 | 3045 | TGTTACCTTGGATCATTATCC | 4027 | CCTATTACTAGGTTCCATTGT |
| NC_045512.2_21mer_win1_26577 | 26577 | 26597 | 3046 | GTTACCTTGGATCATTATCCA | 4028 | ACCTATTACTAGGTTCCATTG |
| NC_045512.2_21mer_win1_26578 | 26578 | 26598 | 3047 | TTACCTTGGATCATTATCCAA | 4029 | AACCTATTACTAGGTTCCATT |
| NC_045512.2_21mer_win1_26579 | 26579 | 26599 | 3048 | TACCTTGGATCATTATCCAAA | 4030 | AAACCTATTACTAGGTTCCAT |
| NC_045512.2_21mer_win1_26580 | 26580 | 26600 | 3049 | ACCTTGGATCATTATCCAAAG | 4031 | GAAACCTATTACTAGGTTCCA |
| NC_045512.2_21mer_win1_27033 | 27033 | 27053 | 3050 | CGATGTAGTGCTTGCGAAAGA | 4032 | AGAAAGCGTTCGTGATGTAGC |
| NC_045512.2_21mer_win1_27034 | 27034 | 27054 | 3051 | GATGTAGTGCTTGCGAAAGAA | 4033 | AAGAAAGCGTTCGTGATGTAG |
| NC_045512.2_21mer_win1_27035 | 27035 | 27055 | 3052 | ATGTAGTGCTTGCGAAAGAAT | 4034 | TAAGAAAGCGTTCGTGATGTA |
| NC_045512.2_21mer_win1_27036 | 27036 | 27056 | 3053 | TGTAGTGCTTGCGAAAGAATA | 4035 | ATAAGAAAGCGTTCGTGATGT |
| NC_045512.2_21mer_win1_27037 | 27037 | 27057 | 3054 | GTAGTGCTTGCGAAAGAATAA | 4036 | AATAAGAAAGCGTTCGTGATG |
| NC_045512.2_21mer_win1_27038 | 27038 | 27058 | 3055 | TAGTGCTTGCGAAAGAATAAT | 4037 | TAATAAGAAAGCGTTCGTGAT |
| NC_045512.2_21mer_win1_27039 | 27039 | 27059 | 3056 | AGTGCTTGCGAAAGAATAATG | 4038 | GTAATAAGAAAGCGTTCGTGA |
| NC_045512.2_21mer_win1_27040 | 27040 | 27060 | 3057 | GTGCTTGCGAAAGAATAATGT | 4039 | TGTAATAAGAAAGCGTTCGTG |
| NC_045512.2_21mer_win1_27041 | 27041 | 27061 | 3058 | TGCTTGCGAAAGAATAATGTT | 4040 | TTGTAATAAGAAAGCGTTCGT |
| NC_045512.2_21mer_win1_27042 | 27042 | 27062 | 3059 | GCTTGCGAAAGAATAATGTTT | 4041 | TTTGTAATAAGAAAGCGTTCG |
| NC_045512.2_21mer_win1_27043 | 27043 | 27063 | 3060 | CTTGCGAAAGAATAATGTTTA | 4042 | ATTTGTAATAAGAAAGCGTTC |
| NC_045512.2_21mer_win1_27044 | 27044 | 27064 | 3061 | TTGCGAAAGAATAATGTTTAA | 4043 | AATTTGTAATAAGAAAGCGTT |
| NC_045512.2_21mer_win1_27183 | 27183 | 27203 | 3062 | CATGTCATTCACTGTTGTCTA | 4044 | ATCTGTTGTCACTTACTGTAC |
| NC_045512.2_21mer_win1_27184 | 27184 | 27204 | 3063 | ATGTCATTCACTGTTGTCTAC | 4045 | CATCTGTTGTCACTTACTGTA |
| NC_045512.2_21mer_win1_27185 | 27185 | 27205 | 3064 | TGTCATTCACTGTTGTCTACA | 4046 | ACATCTGTTGTCACTTACTGT |
| NC_045512.2_21mer_win1_27186 | 27186 | 27206 | 3065 | GTCATTCACTGTTGTCTACAA | 4047 | AACATCTGTTGTCACTTACTG |
| NC_045512.2_21mer_win1_27187 | 27187 | 27207 | 3066 | TCATTCACTGTTGTCTACAAA | 4048 | AAACATCTGTTGTCACTTACT |
| NC_045512.2_21mer_win1_27188 | 27188 | 27208 | 3067 | CATTCACTGTTGTCTACAAAG | 4049 | GAAACATCTGTTGTCACTTAC |
| NC_045512.2_21mer_win1_27189 | 27189 | 27209 | 3068 | ATTCACTGTTGTCTACAAAGT | 4050 | TGAAACATCTGTTGTCACTTA |
| NC_045512.2_21mer_win1_27190 | 27190 | 27210 | 3069 | TTCACTGTTGTCTACAAAGTA | 4051 | ATGAAACATCTGTTGTCACTT |
| NC_045512.2_21mer_win1_27191 | 27191 | 27211 | 3070 | TCACTGTTGTCTACAAAGTAG | 4052 | GATGAAACATCTGTTGTCACT |
| NC_045512.2_21mer_win1_27192 | 27192 | 27212 | 3071 | CACTGTTGTCTACAAAGTAGA | 4053 | AGATGAAACATCTGTTGTCAC |
| NC_045512.2_21mer_win1_27382 | 27382 | 27402 | 3072 | CTAATTTGCTTGTACTTTTAA | 4054 | AATTTTCATGTTCGTTTAATC |
| NC_045512.2_21mer_win1_27383 | 27383 | 27403 | 3073 | TAATTTGCTTGTACTTTTAAT | 4055 | TAATTTTCATGTTCGTTTAAT |
| NC_045512.2_21mer_win1_27384 | 27384 | 27404 | 3074 | AATTTGCTTGTACTTTTAATA | 4056 | ATAATTTTCATGTTCGTTTAA |
| NC_045512.2_21mer_win1_27385 | 27385 | 27405 | 3075 | ATTTGCTTGTACTTTTAATAA | 4057 | AATAATTTTCATGTTCGTTTA |
| NC_045512.2_21mer_win1_27386 | 27386 | 27406 | 3076 | TTTGCTTGTACTTTTAATAAG | 4058 | GAATAATTTTCATGTTCGTTT |
| NC_045512.2_21mer_win1_27387 | 27387 | 27407 | 3077 | TTGCTTGTACTTTTAATAAGA | 4059 | AGAATAATTTTCATGTTCGTT |
| NC_045512.2_21mer_win1_27511 | 27511 | 27531 | 3078 | ATGCTCCCGTTAAGTGGTAAA | 4060 | AAATGGTGAATTGCCCTCGTA |
| NC_045512.2_21mer_win1_27512 | 27512 | 27532 | 3079 | TGCTCCCGTTAAGTGGTAAAG | 4061 | GAAATGGTGAATTGCCCTCGT |
| NC_045512.2_21mer_win1_27513 | 27513 | 27533 | 3080 | GCTCCCGTTAAGTGGTAAAGT | 4062 | TGAAATGGTGAATTGCCCTCG |
| NC_045512.2_21mer_win1_27771 | 27771 | 27791 | 3081 | AATTAACTGAAGATAAACACG | 4063 | GCACAAATAGAAGTCAATTAA |
| NC_045512.2_21mer_win1_27772 | 27772 | 27792 | 3082 | ATTAACTGAAGATAAACACGA | 4064 | AGCACAAATAGAAGTCAATTA |
| NC_045512.2_21mer_win1_27773 | 27773 | 27793 | 3083 | TTAACTGAAGATAAACACGAA | 4065 | AAGCACAAATAGAAGTCAATT |
| NC_045512.2_21mer_win1_27774 | 27774 | 27794 | 3084 | TAACTGAAGATAAACACGAAA | 4066 | AAAGCACAAATAGAAGTCAAT |
| NC_045512.2_21mer_win1_27775 | 27775 | 27795 | 3085 | AACTGAAGATAAACACGAAAA | 4067 | AAAAGCACAAATAGAAGTCAA |
| NC_045512.2_21mer_win1_27776 | 27776 | 27796 | 3086 | ACTGAAGATAAACACGAAAAA | 4068 | AAAAAGCACAAATAGAAGTCA |
| NC_045512.2_21mer_win1_27777 | 27777 | 27797 | 3087 | CTGAAGATAAACACGAAAAAT | 4069 | TAAAAAGCACAAATAGAAGTC |
| NC_045512.2_21mer_win1_27778 | 27778 | 27798 | 3088 | TGAAGATAAACACGAAAAATC | 4070 | CTAAAAAGCACAAATAGAAGT |
| NC_045512.2_21mer_win1_27779 | 27779 | 27799 | 3089 | GAAGATAAACACGAAAAATCG | 4071 | GCTAAAAAGCACAAATAGAAG |
| NC_045512.2_21mer_win1_27780 | 27780 | 27800 | 3090 | AAGATAAACACGAAAAATCGG | 4072 | GGCTAAAAAGCACAAATAGAA |
| NC_045512.2_21mer_win1_27781 | 27781 | 27801 | 3091 | AGATAAACACGAAAAATCGGA | 4073 | AGGCTAAAAAGCACAAATAGA |
| NC_045512.2_21mer_win1_27782 | 27782 | 27802 | 3092 | GATAAACACGAAAAATCGGAA | 4074 | AAGGCTAAAAAGCACAAATAG |
| NC_045512.2_21mer_win1_27783 | 27783 | 27803 | 3093 | ATAAACACGAAAAATCGGAAA | 4075 | AAAGGCTAAAAAGCACAAATA |
| NC_045512.2_21mer_win1_27784 | 27784 | 27804 | 3094 | TAAACACGAAAAATCGGAAAG | 4076 | GAAAGGCTAAAAAGCACAAAT |
| NC_045512.2_21mer_win1_27785 | 27785 | 27805 | 3095 | AAACACGAAAAATCGGAAAGA | 4077 | AGAAAGGCTAAAAAGCACAAA |
| NC_045512.2_21mer_win1_27786 | 27786 | 27806 | 3096 | AACACGAAAAATCGGAAAGAC | 4078 | CAGAAAGGCTAAAAAGCACAA |
| NC_045512.2_21mer_win1_27787 | 27787 | 27807 | 3097 | ACACGAAAAATCGGAAAGACG | 4079 | GCAGAAAGGCTAAAAAGCACA |
| NC_045512.2_21mer_win1_27788 | 27788 | 27808 | 3098 | CACGAAAAATCGGAAAGACGA | 4080 | AGCAGAAAGGCTAAAAAGCAC |
| NC_045512.2_21mer_win1_27789 | 27789 | 27809 | 3099 | ACGAAAAATCGGAAAGACGAT | 4081 | TAGCAGAAAGGCTAAAAAGCA |
| NC_045512.2_21mer_win1_27790 | 27790 | 27810 | 3100 | CGAAAAATCGGAAAGACGATA | 4082 | ATAGCAGAAAGGCTAAAAAGC |
| NC_045512.2_21mer_win1_27791 | 27791 | 27811 | 3101 | GAAAAATCGGAAAGACGATAA | 4083 | AATAGCAGAAAGGCTAAAAAG |
| NC_045512.2_21mer_win1_27792 | 27792 | 27812 | 3102 | AAAAATCGGAAAGACGATAAG | 4084 | GAATAGCAGAAAGGCTAAAAA |
| NC_045512.2_21mer_win1_27793 | 27793 | 27813 | 3103 | AAAATCGGAAAGACGATAAGG | 4085 | GGAATAGCAGAAAGGCTAAAA |
| NC_045512.2_21mer_win1_27794 | 27794 | 27814 | 3104 | AAATCGGAAAGACGATAAGGA | 4086 | AGGAATAGCAGAAAGGCTAAA |
| NC_045512.2_21mer_win1_27795 | 27795 | 27815 | 3105 | AATCGGAAAGACGATAAGGAA | 4087 | AAGGAATAGCAGAAAGGCTAA |
| NC_045512.2_21mer_win1_27796 | 27796 | 27816 | 3106 | ATCGGAAAGACGATAAGGAAC | 4088 | CAAGGAATAGCAGAAAGGCTA |
| NC_045512.2_21mer_win1_27797 | 27797 | 27817 | 3107 | TCGGAAAGACGATAAGGAACA | 4089 | ACAAGGAATAGCAGAAAGGCT |
| NC_045512.2_21mer_win1_27798 | 27798 | 27818 | 3108 | CGGAAAGACGATAAGGAACAA | 4090 | AACAAGGAATAGCAGAAAGGC |
| NC_045512.2_21mer_win1_28270 | 28270 | 28290 | 3109 | ATTTTACAGACTATTACCTGG | 4091 | GGTCCATTATCAGACATTTTA |
| NC_045512.2_21mer_win1_28271 | 28271 | 28291 | 3110 | TTTTACAGACTATTACCTGGG | 4092 | GGGTCCATTATCAGACATTTT |
| NC_045512.2_21mer_win1_28272 | 28272 | 28292 | 3111 | TTTACAGACTATTACCTGGGG | 4093 | GGGGTCCATTATCAGACATTT |
| NC_045512.2_21mer_win1_28273 | 28273 | 28293 | 3112 | TTACAGACTATTACCTGGGGT | 4094 | TGGGGTCCATTATCAGACATT |
| NC_045512.2_21mer_win1_28274 | 28274 | 28294 | 3113 | TACAGACTATTACCTGGGGTT | 4095 | TTGGGGTCCATTATCAGACAT |
| NC_045512.2_21mer_win1_28275 | 28275 | 28295 | 3114 | ACAGACTATTACCTGGGGTTT | 4096 | TTTGGGGTCCATTATCAGACA |
| NC_045512.2_21mer_win1_28276 | 28276 | 28296 | 3115 | CAGACTATTACCTGGGGTTTT | 4097 | TTTTGGGGTCCATTATCAGAC |
| NC_045512.2_21mer_win1_28397 | 28397 | 28417 | 3116 | GGGGTTCCAAATGGGTTATTA | 4098 | ATTATTGGGTAAACCTTGGGG |
| NC_045512.2_21mer_win1_28398 | 28398 | 28418 | 3117 | GGGTTCCAAATGGGTTATTAT | 4099 | TATTATTGGGTAAACCTTGGG |
| NC_045512.2_21mer_win1_28399 | 28399 | 28419 | 3118 | GGTTCCAAATGGGTTATTATG | 4100 | GTATTATTGGGTAAACCTTGG |
| NC_045512.2_21mer_win1_28400 | 28400 | 28420 | 3119 | GTTCCAAATGGGTTATTATGA | 4101 | AGTATTATTGGGTAAACCTTG |
| NC_045512.2_21mer_win1_28401 | 28401 | 28421 | 3120 | TTCCAAATGGGTTATTATGAC | 4102 | CAGTATTATTGGGTAAACCTT |
| NC_045512.2_21mer_win1_28402 | 28402 | 28422 | 3121 | TCCAAATGGGTTATTATGACG | 4103 | GCAGTATTATTGGGTAAACCT |
| NC_045512.2_21mer_win1_28403 | 28403 | 28423 | 3122 | CCAAATGGGTTATTATGACGC | 4104 | CGCAGTATTATTGGGTAAACC |
| NC_045512.2_21mer_win1_28404 | 28404 | 28424 | 3123 | CAAATGGGTTATTATGACGCA | 4105 | ACGCAGTATTATTGGGTAAAC |
| NC_045512.2_21mer_win1_28405 | 28405 | 28425 | 3124 | AAATGGGTTATTATGACGCAG | 4106 | GACGCAGTATTATTGGGTAAA |
| NC_045512.2_21mer_win1_28406 | 28406 | 28426 | 3125 | AATGGGTTATTATGACGCAGA | 4107 | AGACGCAGTATTATTGGGTAA |
| NC_045512.2_21mer_win1_28407 | 28407 | 28427 | 3126 | ATGGGTTATTATGACGCAGAA | 4108 | AAGACGCAGTATTATTGGGTA |
| NC_045512.2_21mer_win1_28408 | 28408 | 28428 | 3127 | TGGGTTATTATGACGCAGAAC | 4109 | CAAGACGCAGTATTATTGGGT |
| NC_045512.2_21mer_win1_28409 | 28409 | 28429 | 3128 | GGGTTATTATGACGCAGAACC | 4110 | CCAAGACGCAGTATTATTGGG |
| NC_045512.2_21mer_win1_28410 | 28410 | 28430 | 3129 | GGTTATTATGACGCAGAACCA | 4111 | ACCAAGACGCAGTATTATTGG |
| NC_045512.2_21mer_win1_28411 | 28411 | 28431 | 3130 | GTTATTATGACGCAGAACCAA | 4112 | AACCAAGACGCAGTATTATTG |
| NC_045512.2_21mer_win1_28412 | 28412 | 28432 | 3131 | TTATTATGACGCAGAACCAAG | 4113 | GAACCAAGACGCAGTATTATT |
| NC_045512.2_21mer_win1_28413 | 28413 | 28433 | 3132 | TATTATGACGCAGAACCAAGT | 4114 | TGAACCAAGACGCAGTATTAT |
| NC_045512.2_21mer_win1_28414 | 28414 | 28434 | 3133 | ATTATGACGCAGAACCAAGTG | 4115 | GTGAACCAAGACGCAGTATTA |
| NC_045512.2_21mer_win1_28513 | 28513 | 28533 | 3134 | TCTACTGGTTTAACCGATGAT | 4116 | TAGTAGCCAATTTGGTCATCT |
| NC_045512.2_21mer_win1_28514 | 28514 | 28534 | 3135 | CTACTGGTTTAACCGATGATG | 4117 | GTAGTAGCCAATTTGGTCATC |
| NC_045512.2_21mer_win1_28515 | 28515 | 28535 | 3136 | TACTGGTTTAACCGATGATGG | 4118 | GGTAGTAGCCAATTTGGTCAT |
| NC_045512.2_21mer_win1_28516 | 28516 | 28536 | 3137 | ACTGGTTTAACCGATGATGGC | 4119 | CGGTAGTAGCCAATTTGGTCA |
| NC_045512.2_21mer_win1_28517 | 28517 | 28537 | 3138 | CTGGTTTAACCGATGATGGCT | 4120 | TCGGTAGTAGCCAATTTGGTC |
| NC_045512.2_21mer_win1_28518 | 28518 | 28538 | 3139 | TGGTTTAACCGATGATGGCTT | 4121 | TTCGGTAGTAGCCAATTTGGT |
| NC_045512.2_21mer_win1_28519 | 28519 | 28539 | 3140 | GGTTTAACCGATGATGGCTTC | 4122 | CTTCGGTAGTAGCCAATTTGG |
| NC_045512.2_21mer_win1_28520 | 28520 | 28540 | 3141 | GTTTAACCGATGATGGCTTCT | 4123 | TCTTCGGTAGTAGCCAATTTG |
| NC_045512.2_21mer_win1_28521 | 28521 | 28541 | 3142 | TTTAACCGATGATGGCTTCTC | 4124 | CTCTTCGGTAGTAGCCAATTT |
| NC_045512.2_21mer_win1_28522 | 28522 | 28542 | 3143 | TTAACCGATGATGGCTTCTCG | 4125 | GCTCTTCGGTAGTAGCCAATT |
| NC_045512.2_21mer_win1_28523 | 28523 | 28543 | 3144 | TAACCGATGATGGCTTCTCGA | 4126 | AGCTCTTCGGTAGTAGCCAAT |
| NC_045512.2_21mer_win1_28524 | 28524 | 28544 | 3145 | AACCGATGATGGCTTCTCGAT | 4127 | TAGCTCTTCGGTAGTAGCCAA |
| NC_045512.2_21mer_win1_28525 | 28525 | 28545 | 3146 | ACCGATGATGGCTTCTCGATG | 4128 | GTAGCTCTTCGGTAGTAGCCA |
| NC_045512.2_21mer_win1_28526 | 28526 | 28546 | 3147 | CCGATGATGGCTTCTCGATGG | 4129 | GGTAGCTCTTCGGTAGTAGCC |
| NC_045512.2_21mer_win1_28706 | 28706 | 28726 | 3148 | GTGTAACCGTGGGCGTTAGGA | 4130 | AGGATTGCGGGTGCCAATGTG |
| NC_045512.2_21mer_win1_28744 | 28744 | 28764 | 3149 | GCACGATGTTGAAGGAGTTCC | 4131 | CCTTGAGGAAGTTGTAGCACG |
| NC_045512.2_21mer_win1_28745 | 28745 | 28765 | 3150 | CACGATGTTGAAGGAGTTCCT | 4132 | TCCTTGAGGAAGTTGTAGCAC |
| NC_045512.2_21mer_win1_28746 | 28746 | 28766 | 3151 | ACGATGTTGAAGGAGTTCCTT | 4133 | TTCCTTGAGGAAGTTGTAGCA |
| NC_045512.2_21mer_win1_28747 | 28747 | 28767 | 3152 | CGATGTTGAAGGAGTTCCTTG | 4134 | GTTCCTTGAGGAAGTTGTAGC |
| NC_045512.2_21mer_win1_28748 | 28748 | 28768 | 3153 | GATGTTGAAGGAGTTCCTTGT | 4135 | TGTTCCTTGAGGAAGTTGTAG |
| NC_045512.2_21mer_win1_28749 | 28749 | 28769 | 3154 | ATGTTGAAGGAGTTCCTTGTT | 4136 | TTGTTCCTTGAGGAAGTTGTA |
| NC_045512.2_21mer_win1_28750 | 28750 | 28770 | 3155 | TGTTGAAGGAGTTCCTTGTTG | 4137 | GTTGTTCCTTGAGGAAGTTGT |
| NC_045512.2_21mer_win1_28751 | 28751 | 28771 | 3156 | GTTGAAGGAGTTCCTTGTTGT | 4138 | TGTTGTTCCTTGAGGAAGTTG |
| NC_045512.2_21mer_win1_28752 | 28752 | 28772 | 3157 | TTGAAGGAGTTCCTTGTTGTA | 4139 | ATGTTGTTCCTTGAGGAAGTT |
| NC_045512.2_21mer_win1_28753 | 28753 | 28773 | 3158 | TGAAGGAGTTCCTTGTTGTAA | 4140 | AATGTTGTTCCTTGAGGAAGT |
| NC_045512.2_21mer_win1_28754 | 28754 | 28774 | 3159 | GAAGGAGTTCCTTGTTGTAAC | 4141 | CAATGTTGTTCCTTGAGGAAG |
| NC_045512.2_21mer_win1_28755 | 28755 | 28775 | 3160 | AAGGAGTTCCTTGTTGTAACG | 4142 | GCAATGTTGTTCCTTGAGGAA |
| NC_045512.2_21mer_win1_28756 | 28756 | 28776 | 3161 | AGGAGTTCCTTGTTGTAACGG | 4143 | GGCAATGTTGTTCCTTGAGGA |
| NC_045512.2_21mer_win1_28757 | 28757 | 28777 | 3162 | GGAGTTCCTTGTTGTAACGGT | 4144 | TGGCAATGTTGTTCCTTGAGG |
| NC_045512.2_21mer_win1_28758 | 28758 | 28778 | 3163 | GAGTTCCTTGTTGTAACGGTT | 4145 | TTGGCAATGTTGTTCCTTGAG |
| NC_045512.2_21mer_win1_28759 | 28759 | 28779 | 3164 | AGTTCCTTGTTGTAACGGTTT | 4146 | TTTGGCAATGTTGTTCCTTGA |
| NC_045512.2_21mer_win1_28760 | 28760 | 28780 | 3165 | GTTCCTTGTTGTAACGGTTTT | 4147 | TTTTGGCAATGTTGTTCCTTG |
| NC_045512.2_21mer_win1_28761 | 28761 | 28781 | 3166 | TTCCTTGTTGTAACGGTTTTC | 4148 | CTTTTGGCAATGTTGTTCCTT |
| NC_045512.2_21mer_win1_28762 | 28762 | 28782 | 3167 | TCCTTGTTGTAACGGTTTTCC | 4149 | CCTTTTGGCAATGTTGTTCCT |
| NC_045512.2_21mer_win1_28763 | 28763 | 28783 | 3168 | CCTTGTTGTAACGGTTTTCCG | 4150 | GCCTTTTGGCAATGTTGTTCC |
| NC_045512.2_21mer_win1_28764 | 28764 | 28784 | 3169 | CTTGTTGTAACGGTTTTCCGA | 4151 | AGCCTTTTGGCAATGTTGTTC |
| NC_045512.2_21mer_win1_28765 | 28765 | 28785 | 3170 | TTGTTGTAACGGTTTTCCGAA | 4152 | AAGCCTTTTGGCAATGTTGTT |
| NC_045512.2_21mer_win1_28766 | 28766 | 28786 | 3171 | TGTTGTAACGGTTTTCCGAAG | 4153 | GAAGCCTTTTGGCAATGTTGT |
| NC_045512.2_21mer_win1_28767 | 28767 | 28787 | 3172 | GTTGTAACGGTTTTCCGAAGA | 4154 | AGAAGCCTTTTGGCAATGTTG |
| NC_045512.2_21mer_win1_28768 | 28768 | 28788 | 3173 | TTGTAACGGTTTTCCGAAGAT | 4155 | TAGAAGCCTTTTGGCAATGTT |
| NC_045512.2_21mer_win1_28769 | 28769 | 28789 | 3174 | TGTAACGGTTTTCCGAAGATG | 4156 | GTAGAAGCCTTTTGGCAATGT |
| NC_045512.2_21mer_win1_28770 | 28770 | 28790 | 3175 | GTAACGGTTTTCCGAAGATGC | 4157 | CGTAGAAGCCTTTTGGCAATG |
| NC_045512.2_21mer_win1_28771 | 28771 | 28791 | 3176 | TAACGGTTTTCCGAAGATGCG | 4158 | GCGTAGAAGCCTTTTGGCAAT |
| NC_045512.2_21mer_win1_28772 | 28772 | 28792 | 3177 | AACGGTTTTCCGAAGATGCGT | 4159 | TGCGTAGAAGCCTTTTGGCAA |
| NC_045512.2_21mer_win1_28773 | 28773 | 28793 | 3178 | ACGGTTTTCCGAAGATGCGTC | 4160 | CTGCGTAGAAGCCTTTTGGCA |
| NC_045512.2_21mer_win1_28774 | 28774 | 28794 | 3179 | CGGTTTTCCGAAGATGCGTCT | 4161 | TCTGCGTAGAAGCCTTTTGGC |
| NC_045512.2_21mer_win1_28799 | 28799 | 28819 | 3180 | TCGTCTCCGCCGTCAGTTCGG | 4162 | GGCTTGACTGCCGCCTCTGCT |
| NC_045512.2_21mer_win1_28800 | 28800 | 28820 | 3181 | CGTCTCCGCCGTCAGTTCGGA | 4163 | AGGCTTGACTGCCGCCTCTGC |
| NC_045512.2_21mer_win1_28801 | 28801 | 28821 | 3182 | GTCTCCGCCGTCAGTTCGGAG | 4164 | GAGGCTTGACTGCCGCCTCTG |
| NC_045512.2_21mer_win1_28802 | 28802 | 28822 | 3183 | TCTCCGCCGTCAGTTCGGAGA | 4165 | AGAGGCTTGACTGCCGCCTCT |
| NC_045512.2_21mer_win1_28803 | 28803 | 28823 | 3184 | CTCCGCCGTCAGTTCGGAGAA | 4166 | AAGAGGCTTGACTGCCGCCTC |
| NC_045512.2_21mer_win1_28804 | 28804 | 28824 | 3185 | TCCGCCGTCAGTTCGGAGAAG | 4167 | GAAGAGGCTTGACTGCCGCCT |
| NC_045512.2_21mer_win1_28805 | 28805 | 28825 | 3186 | CCGCCGTCAGTTCGGAGAAGA | 4168 | AGAAGAGGCTTGACTGCCGCC |
| NC_045512.2_21mer_win1_28806 | 28806 | 28826 | 3187 | CGCCGTCAGTTCGGAGAAGAG | 4169 | GAGAAGAGGCTTGACTGCCGC |
| NC_045512.2_21mer_win1_28807 | 28807 | 28827 | 3188 | GCCGTCAGTTCGGAGAAGAGC | 4170 | CGAGAAGAGGCTTGACTGCCG |
| NC_045512.2_21mer_win1_28946 | 28946 | 28966 | 3189 | CTGTCTAACTTGGTCGAACTC | 4171 | CTCAAGCTGGTTCAATCTGTC |
| NC_045512.2_21mer_win1_28947 | 28947 | 28967 | 3190 | TGTCTAACTTGGTCGAACTCT | 4172 | TCTCAAGCTGGTTCAATCTGT |
| NC_045512.2_21mer_win1_28948 | 28948 | 28968 | 3191 | GTCTAACTTGGTCGAACTCTC | 4173 | CTCTCAAGCTGGTTCAATCTG |
| NC_045512.2_21mer_win1_28949 | 28949 | 28969 | 3192 | TCTAACTTGGTCGAACTCTCG | 4174 | GCTCTCAAGCTGGTTCAATCT |
| NC_045512.2_21mer_win1_28950 | 28950 | 28970 | 3193 | CTAACTTGGTCGAACTCTCGT | 4175 | TGCTCTCAAGCTGGTTCAATC |
| NC_045512.2_21mer_win1_28951 | 28951 | 28971 | 3194 | TAACTTGGTCGAACTCTCGTT | 4176 | TTGCTCTCAAGCTGGTTCAAT |
| NC_045512.2_21mer_win1_28952 | 28952 | 28972 | 3195 | AACTTGGTCGAACTCTCGTTT | 4177 | TTTGCTCTCAAGCTGGTTCAA |
| NC_045512.2_21mer_win1_28976 | 28976 | 28996 | 3196 | AGACCATTTCCGGTTGTTGTT | 4178 | TTGTTGTTGGCCTTTACCAGA |
| NC_045512.2_21mer_win1_28977 | 28977 | 28997 | 3197 | GACCATTTCCGGTTGTTGTTG | 4179 | GTTGTTGTTGGCCTTTACCAG |
| NC_045512.2_21mer_win1_28978 | 28978 | 28998 | 3198 | ACCATTTCCGGTTGTTGTTGT | 4180 | TGTTGTTGTTGGCCTTTACCA |
| NC_045512.2_21mer_win1_28979 | 28979 | 28999 | 3199 | CCATTTCCGGTTGTTGTTGTT | 4181 | TTGTTGTTGTTGGCCTTTACC |
| NC_045512.2_21mer_win1_28980 | 28980 | 29000 | 3200 | CATTTCCGGTTGTTGTTGTTC | 4182 | CTTGTTGTTGTTGGCCTTTAC |
| NC_045512.2_21mer_win1_28981 | 28981 | 29001 | 3201 | ATTTCCGGTTGTTGTTGTTCC | 4183 | CCTTGTTGTTGTTGGCCTTTA |
| NC_045512.2_21mer_win1_28982 | 28982 | 29002 | 3202 | TTTCCGGTTGTTGTTGTTCCG | 4184 | GCCTTGTTGTTGTTGGCCTTT |
| NC_045512.2_21mer_win1_28983 | 28983 | 29003 | 3203 | TTCCGGTTGTTGTTGTTCCGG | 4185 | GGCCTTGTTGTTGTTGGCCTT |
| NC_045512.2_21mer_win1_28984 | 28984 | 29004 | 3204 | TCCGGTTGTTGTTGTTCCGGT | 4186 | TGGCCTTGTTGTTGTTGGCCT |
| NC_045512.2_21mer_win1_28985 | 28985 | 29005 | 3205 | CCGGTTGTTGTTGTTCCGGTT | 4187 | TTGGCCTTGTTGTTGTTGGCC |
| NC_045512.2_21mer_win1_28986 | 28986 | 29006 | 3206 | CGGTTGTTGTTGTTCCGGTTT | 4188 | TTTGGCCTTGTTGTTGTTGGC |
| NC_045512.2_21mer_win1_28987 | 28987 | 29007 | 3207 | GGTTGTTGTTGTTCCGGTTTG | 4189 | GTTTGGCCTTGTTGTTGTTGG |
| NC_045512.2_21mer_win1_28988 | 28988 | 29008 | 3208 | GTTGTTGTTGTTCCGGTTTGA | 4190 | AGTTTGGCCTTGTTGTTGTTG |
| NC_045512.2_21mer_win1_28989 | 28989 | 29009 | 3209 | TTGTTGTTGTTCCGGTTTGAC | 4191 | CAGTTTGGCCTTGTTGTTGTT |
| NC_045512.2_21mer_win1_28990 | 28990 | 29010 | 3210 | TGTTGTTGTTCCGGTTTGACA | 4192 | ACAGTTTGGCCTTGTTGTTGT |
| NC_045512.2_21mer_win1_28991 | 28991 | 29011 | 3211 | GTTGTTGTTCCGGTTTGACAG | 4193 | GACAGTTTGGCCTTGTTGTTG |
| NC_045512.2_21mer_win1_28992 | 28992 | 29012 | 3212 | TTGTTGTTCCGGTTTGACAGT | 4194 | TGACAGTTTGGCCTTGTTGTT |
| NC_045512.2_21mer_win1_28993 | 28993 | 29013 | 3213 | TGTTGTTCCGGTTTGACAGTG | 4195 | GTGACAGTTTGGCCTTGTTGT |
| NC_045512.2_21mer_win1_28994 | 28994 | 29014 | 3214 | GTTGTTCCGGTTTGACAGTGA | 4196 | AGTGACAGTTTGGCCTTGTTG |
| NC_045512.2_21mer_win1_28995 | 28995 | 29015 | 3215 | TTGTTCCGGTTTGACAGTGAT | 4197 | TAGTGACAGTTTGGCCTTGTT |
| NC_045512.2_21mer_win1_28996 | 28996 | 29016 | 3216 | TGTTCCGGTTTGACAGTGATT | 4198 | TTAGTGACAGTTTGGCCTTGT |
| NC_045512.2_21mer_win1_28997 | 28997 | 29017 | 3217 | GTTCCGGTTTGACAGTGATTC | 4199 | CTTAGTGACAGTTTGGCCTTG |
| NC_045512.2_21mer_win1_28998 | 28998 | 29018 | 3218 | TTCCGGTTTGACAGTGATTCT | 4200 | TCTTAGTGACAGTTTGGCCTT |
| NC_045512.2_21mer_win1_28999 | 28999 | 29019 | 3219 | TCCGGTTTGACAGTGATTCTT | 4201 | TTCTTAGTGACAGTTTGGCCT |
| NC_045512.2_21mer_win1_29000 | 29000 | 29020 | 3220 | CCGGTTTGACAGTGATTCTTT | 4202 | TTTCTTAGTGACAGTTTGGCC |
| NC_045512.2_21mer_win1_29001 | 29001 | 29021 | 3221 | CGGTTTGACAGTGATTCTTTA | 4203 | ATTTCTTAGTGACAGTTTGGC |
| NC_045512.2_21mer_win1_29002 | 29002 | 29022 | 3222 | GGTTTGACAGTGATTCTTTAG | 4204 | GATTTCTTAGTGACAGTTTGG |
| NC_045512.2_21mer_win1_29003 | 29003 | 29023 | 3223 | GTTTGACAGTGATTCTTTAGA | 4205 | AGATTTCTTAGTGACAGTTTG |
| NC_045512.2_21mer_win1_29004 | 29004 | 29024 | 3224 | TTTGACAGTGATTCTTTAGAC | 4206 | CAGATTTCTTAGTGACAGTTT |
| NC_045512.2_21mer_win1_29005 | 29005 | 29025 | 3225 | TTGACAGTGATTCTTTAGACG | 4207 | GCAGATTTCTTAGTGACAGTT |
| NC_045512.2_21mer_win1_29006 | 29006 | 29026 | 3226 | TGACAGTGATTCTTTAGACGA | 4208 | AGCAGATTTCTTAGTGACAGT |
| NC_045512.2_21mer_win1_29007 | 29007 | 29027 | 3227 | GACAGTGATTCTTTAGACGAC | 4209 | CAGCAGATTTCTTAGTGACAG |
| NC_045512.2_21mer_win1_29008 | 29008 | 29028 | 3228 | ACAGTGATTCTTTAGACGACG | 4210 | GCAGCAGATTTCTTAGTGACA |
| NC_045512.2_21mer_win1_29009 | 29009 | 29029 | 3229 | CAGTGATTCTTTAGACGACGA | 4211 | AGCAGCAGATTTCTTAGTGAC |
| NC_045512.2_21mer_win1_29010 | 29010 | 29030 | 3230 | AGTGATTCTTTAGACGACGAC | 4212 | CAGCAGCAGATTTCTTAGTGA |
| NC_045512.2_21mer_win1_29011 | 29011 | 29031 | 3231 | GTGATTCTTTAGACGACGACT | 4213 | TCAGCAGCAGATTTCTTAGTG |
| NC_045512.2_21mer_win1_29012 | 29012 | 29032 | 3232 | TGATTCTTTAGACGACGACTC | 4214 | CTCAGCAGCAGATTTCTTAGT |
| NC_045512.2_21mer_win1_29013 | 29013 | 29033 | 3233 | GATTCTTTAGACGACGACTCC | 4215 | CCTCAGCAGCAGATTTCTTAG |
| NC_045512.2_21mer_win1_29014 | 29014 | 29034 | 3234 | ATTCTTTAGACGACGACTCCG | 4216 | GCCTCAGCAGCAGATTTCTTA |
| NC_045512.2_21mer_win1_29144 | 29144 | 29164 | 3235 | GATTAGTCTGTTCCTTGACTA | 4217 | ATCAGTTCCTTGTCTGATTAG |
| NC_045512.2_21mer_win1_29145 | 29145 | 29165 | 3236 | ATTAGTCTGTTCCTTGACTAA | 4218 | AATCAGTTCCTTGTCTGATTA |
| NC_045512.2_21mer_win1_29146 | 29146 | 29166 | 3237 | TTAGTCTGTTCCTTGACTAAT | 4219 | TAATCAGTTCCTTGTCTGATT |
| NC_045512.2_21mer_win1_29147 | 29147 | 29167 | 3238 | TAGTCTGTTCCTTGACTAATG | 4220 | GTAATCAGTTCCTTGTCTGAT |
| NC_045512.2_21mer_win1_29148 | 29148 | 29168 | 3239 | AGTCTGTTCCTTGACTAATGT | 4221 | TGTAATCAGTTCCTTGTCTGA |
| NC_045512.2_21mer_win1_29149 | 29149 | 29169 | 3240 | GTCTGTTCCTTGACTAATGTT | 4222 | TTGTAATCAGTTCCTTGTCTG |
| NC_045512.2_21mer_win1_29150 | 29150 | 29170 | 3241 | TCTGTTCCTTGACTAATGTTT | 4223 | TTTGTAATCAGTTCCTTGTCT |
| NC_045512.2_21mer_win1_29151 | 29151 | 29171 | 3242 | CTGTTCCTTGACTAATGTTTG | 4224 | GTTTGTAATCAGTTCCTTGTC |
| NC_045512.2_21mer_win1_29152 | 29152 | 29172 | 3243 | TGTTCCTTGACTAATGTTTGT | 4225 | TGTTTGTAATCAGTTCCTTGT |
| NC_045512.2_21mer_win1_29174 | 29174 | 29194 | 3244 | ACCGGCGTTTAACGTGTTAAA | 4226 | AAATTGTGCAATTTGCGGCCA |
| NC_045512.2_21mer_win1_29175 | 29175 | 29195 | 3245 | CCGGCGTTTAACGTGTTAAAC | 4227 | CAAATTGTGCAATTTGCGGCC |
| NC_045512.2_21mer_win1_29176 | 29176 | 29196 | 3246 | CGGCGTTTAACGTGTTAAACG | 4228 | GCAAATTGTGCAATTTGCGGC |
| NC_045512.2_21mer_win1_29228 | 29228 | 29248 | 3247 | GCGTAACCGTACCTTCAGTGT | 4229 | TGTGACTTCCATGCCAATGCG |
| NC_045512.2_21mer_win1_29229 | 29229 | 29249 | 3248 | CGTAACCGTACCTTCAGTGTG | 4230 | GTGTGACTTCCATGCCAATGC |
| NC_045512.2_21mer_win1_29230 | 29230 | 29250 | 3249 | GTAACCGTACCTTCAGTGTGG | 4231 | GGTGTGACTTCCATGCCAATG |
| NC_045512.2_21mer_win1_29231 | 29231 | 29251 | 3250 | TAACCGTACCTTCAGTGTGGA | 4232 | AGGTGTGACTTCCATGCCAAT |
| NC_045512.2_21mer_win1_29232 | 29232 | 29252 | 3251 | AACCGTACCTTCAGTGTGGAA | 4233 | AAGGTGTGACTTCCATGCCAA |
| NC_045512.2_21mer_win1_29233 | 29233 | 29253 | 3252 | ACCGTACCTTCAGTGTGGAAG | 4234 | GAAGGTGTGACTTCCATGCCA |
| NC_045512.2_21mer_win1_29234 | 29234 | 29254 | 3253 | CCGTACCTTCAGTGTGGAAGC | 4235 | CGAAGGTGTGACTTCCATGCC |
| NC_045512.2_21mer_win1_29235 | 29235 | 29255 | 3254 | CGTACCTTCAGTGTGGAAGCC | 4236 | CCGAAGGTGTGACTTCCATGC |
| NC_045512.2_21mer_win1_29236 | 29236 | 29256 | 3255 | GTACCTTCAGTGTGGAAGCCC | 4237 | CCCGAAGGTGTGACTTCCATG |
| NC_045512.2_21mer_win1_29237 | 29237 | 29257 | 3256 | TACCTTCAGTGTGGAAGCCCT | 4238 | TCCCGAAGGTGTGACTTCCAT |
| NC_045512.2_21mer_win1_29238 | 29238 | 29258 | 3257 | ACCTTCAGTGTGGAAGCCCTT | 4239 | TTCCCGAAGGTGTGACTTCCA |
| NC_045512.2_21mer_win1_29239 | 29239 | 29259 | 3258 | CCTTCAGTGTGGAAGCCCTTG | 4240 | GTTCCCGAAGGTGTGACTTCC |
| NC_045512.2_21mer_win1_29285 | 29285 | 29305 | 3259 | TTTAACCTACTGTTTCTAGGT | 4241 | TGGATCTTTGTCATCCAATTT |
| NC_045512.2_21mer_win1_29342 | 29342 | 29362 | 3260 | TAACTGCGTATGTTTTGTAAG | 4242 | GAATGTTTTGTATGCGTCAAT |
| NC_045512.2_21mer_win1_29343 | 29343 | 29363 | 3261 | AACTGCGTATGTTTTGTAAGG | 4243 | GGAATGTTTTGTATGCGTCAA |
| NC_045512.2_21mer_win1_29344 | 29344 | 29364 | 3262 | ACTGCGTATGTTTTGTAAGGG | 4244 | GGGAATGTTTTGTATGCGTCA |
| NC_045512.2_21mer_win1_29345 | 29345 | 29365 | 3263 | CTGCGTATGTTTTGTAAGGGT | 4245 | TGGGAATGTTTTGTATGCGTC |
| NC_045512.2_21mer_win1_29346 | 29346 | 29366 | 3264 | TGCGTATGTTTTGTAAGGGTG | 4246 | GTGGGAATGTTTTGTATGCGT |
| NC_045512.2_21mer_win1_29347 | 29347 | 29367 | 3265 | GCGTATGTTTTGTAAGGGTGG | 4247 | GGTGGGAATGTTTTGTATGCG |
| NC_045512.2_21mer_win1_29348 | 29348 | 29368 | 3266 | CGTATGTTTTGTAAGGGTGGT | 4248 | TGGTGGGAATGTTTTGTATGC |
| NC_045512.2_21mer_win1_29349 | 29349 | 29369 | 3267 | GTATGTTTTGTAAGGGTGGTT | 4249 | TTGGTGGGAATGTTTTGTATG |
| NC_045512.2_21mer_win1_29350 | 29350 | 29370 | 3268 | TATGTTTTGTAAGGGTGGTTG | 4250 | GTTGGTGGGAATGTTTTGTAT |
| NC_045512.2_21mer_win1_29351 | 29351 | 29371 | 3269 | ATGTTTTGTAAGGGTGGTTGT | 4251 | TGTTGGTGGGAATGTTTTGTA |
| NC_045512.2_21mer_win1_29352 | 29352 | 29372 | 3270 | TGTTTTGTAAGGGTGGTTGTC | 4252 | CTGTTGGTGGGAATGTTTTGT |
| NC_045512.2_21mer_win1_29353 | 29353 | 29373 | 3271 | GTTTTGTAAGGGTGGTTGTCT | 4253 | TCTGTTGGTGGGAATGTTTTG |
| NC_045512.2_21mer_win1_29354 | 29354 | 29374 | 3272 | TTTTGTAAGGGTGGTTGTCTC | 4254 | CTCTGTTGGTGGGAATGTTTT |
| NC_045512.2_21mer_win1_29355 | 29355 | 29375 | 3273 | TTTGTAAGGGTGGTTGTCTCG | 4255 | GCTCTGTTGGTGGGAATGTTT |
| NC_045512.2_21mer_win1_29356 | 29356 | 29376 | 3274 | TTGTAAGGGTGGTTGTCTCGG | 4256 | GGCTCTGTTGGTGGGAATGTT |
| NC_045512.2_21mer_win1_29357 | 29357 | 29377 | 3275 | TGTAAGGGTGGTTGTCTCGGA | 4257 | AGGCTCTGTTGGTGGGAATGT |
| NC_045512.2_21mer_win1_29358 | 29358 | 29378 | 3276 | GTAAGGGTGGTTGTCTCGGAT | 4258 | TAGGCTCTGTTGGTGGGAATG |
| NC_045512.2_21mer_win1_29359 | 29359 | 29379 | 3277 | TAAGGGTGGTTGTCTCGGATT | 4259 | TTAGGCTCTGTTGGTGGGAAT |
| NC_045512.2_21mer_win1_29360 | 29360 | 29380 | 3278 | AAGGGTGGTTGTCTCGGATTT | 4260 | TTTAGGCTCTGTTGGTGGGAA |
| NC_045512.2_2lmer_win1_29361 | 29361 | 29381 | 3279 | AGGGTGGTTGTCTCGGATTTT | 4261 | TTTTAGGCTCTGTTGGTGGGA |
| NC_045512.2_21mer_win1_29362 | 29362 | 29382 | 3280 | GGGTGGTTGTCTCGGATTTTT | 4262 | TTTTTAGGCTCTGTTGGTGGG |
| NC_045512.2_21mer_win1_29363 | 29363 | 29383 | 3281 | GGTGGTTGTCTCGGATTTTTC | 4263 | CTTTTTAGGCTCTGTTGGTGG |
| NC_045512.2_21mer_win1_29364 | 29364 | 29384 | 3282 | GTGGTTGTCTCGGATTTTTCC | 4264 | CCTTTTTAGGCTCTGTTGGTG |
| NC_045512.2_21mer_win1_29365 | 29365 | 29385 | 3283 | TGGTTGTCTCGGATTTTTCCT | 4265 | TCCTTTTTAGGCTCTGTTGGT |
| NC_045512.2_21mer_win1_29366 | 29366 | 29386 | 3284 | GGTTGTCTCGGATTTTTCCTG | 4266 | GTCCTTTTTAGGCTCTGTTGG |
| NC_045512.2_21mer_win1_29367 | 29367 | 29387 | 3285 | GTTGTCTCGGATTTTTCCTGT | 4267 | TGTCCTTTTTAGGCTCTGTTG |
| NC_045512.2_21mer_win1_29368 | 29368 | 29388 | 3286 | TTGTCTCGGATTTTTCCTGTT | 4268 | TTGTCCTTTTTAGGCTCTGTT |
| NC_045512.2_21mer_win1_29369 | 29369 | 29389 | 3287 | TGTCTCGGATTTTTCCTGTTT | 4269 | TTTGTCCTTTTTAGGCTCTGT |
| NC_045512.2_21mer_win1_29370 | 29370 | 29390 | 3288 | GTCTCGGATTTTTCCTGTTTT | 4270 | TTTTGTCCTTTTTAGGCTCTG |
| NC_045512.2_2lmer_win1_29371 | 29371 | 29391 | 3289 | TCTCGGATTTTTCCTGTTTTT | 4271 | TTTTTGTCCTTTTTAGGCTCT |
| NC_045512.2_21mer_win1_29372 | 29372 | 29392 | 3290 | CTCGGATTTTTCCTGTTTTTC | 4272 | CTTTTTGTCCTTTTTAGGCTC |
| NC_045512.2_21mer_win1_29373 | 29373 | 29393 | 3291 | TCGGATTTTTCCTGTTTTTCT | 4273 | TCTTTTTGTCCTTTTTAGGCT |
| NC_045512.2_21mer_win1_29374 | 29374 | 29394 | 3292 | CGGATTTTTCCTGTTTTTCTT | 4274 | TTCTTTTTGTCCTTTTTAGGC |
| NC_045512.2_21mer_win1_29543 | 29543 | 29563 | 3293 | CTGGTGTGTTCCGTCTACCCG | 4275 | GCCCATCTGCCTTGTGTGGTC |
| NC_045512.2_21mer_win1_29544 | 29544 | 29564 | 3294 | TGGTGTGTTCCGTCTACCCGA | 4276 | AGCCCATCTGCCTTGTGTGGT |
| NC_045512.2_21mer_win1_29545 | 29545 | 29565 | 3295 | GGTGTGTTCCGTCTACCCGAT | 4277 | TAGCCCATCTGCCTTGTGTGG |
| NC_045512.2_21mer_win1_29546 | 29546 | 29566 | 3296 | GTGTGTTCCGTCTACCCGATA | 4278 | ATAGCCCATCTGCCTTGTGTG |
| NC_045512.2_21mer_win1_29598 | 29598 | 29618 | 3297 | TATCAGATGAGAACACGTCTT | 4279 | TTCTGCACAAGAGTAGACTAT |
| NC_045512.2_21mer_win1_29599 | 29599 | 29619 | 3298 | ATCAGATGAGAACACGTCTTA | 4280 | ATTCTGCACAAGAGTAGACTA |
| NC_045512.2_21mer_win1_29600 | 29600 | 29620 | 3299 | TCAGATGAGAACACGTCTTAC | 4281 | CATTCTGCACAAGAGTAGACT |
| NC_045512.2_21mer_win1_29601 | 29601 | 29621 | 3300 | CAGATGAGAACACGTCTTACT | 4282 | TCATTCTGCACAAGAGTAGAC |
| NC_045512.2_21mer_win1_29602 | 29602 | 29622 | 3301 | AGATGAGAACACGTCTTACTT | 4283 | TTCATTCTGCACAAGAGTAGA |
| NC_045512.2_21mer_win1_29603 | 29603 | 29623 | 3302 | GATGAGAACACGTCTTACTTA | 4284 | ATTCATTCTGCACAAGAGTAG |
| NC_045512.2_21mer_win1_29604 | 29604 | 29624 | 3303 | ATGAGAACACGTCTTACTTAA | 4285 | AATTCATTCTGCACAAGAGTA |
| NC_045512.2_21mer_win1_29605 | 29605 | 29625 | 3304 | TGAGAACACGTCTTACTTAAG | 4286 | GAATTCATTCTGCACAAGAGT |
| NC_045512.2_21mer_win1_29606 | 29606 | 29626 | 3305 | GAGAACACGTCTTACTTAAGA | 4287 | AGAATTCATTCTGCACAAGAG |
| NC_045512.2_21mer_win1_29607 | 29607 | 29627 | 3306 | AGAACACGTCTTACTTAAGAG | 4288 | GAGAATTCATTCTGCACAAGA |
| NC_045512.2_21mer_win1_29608 | 29608 | 29628 | 3307 | GAACACGTCTTACTTAAGAGC | 4289 | CGAGAATTCATTCTGCACAAG |
| NC_045512.2_21mer_win1_29609 | 29609 | 29629 | 3308 | AACACGTCTTACTTAAGAGCA | 4290 | ACGAGAATTCATTCTGCACAA |
| NC_045512.2_21mer_win1_29610 | 29610 | 29630 | 3309 | ACACGTCTTACTTAAGAGCAT | 4291 | TACGAGAATTCATTCTGCACA |
| NC_045512.2_21mer_win1_29652 | 29652 | 29672 | 3310 | ATCAATTGAAATTAGAGTGTA | 4292 | ATGTGAGATTAAAGTTAACTA |
| NC_045512.2_21mer_win1_29653 | 29653 | 29673 | 3311 | TCAATTGAAATTAGAGTGTAT | 4293 | TATGTGAGATTAAAGTTAACT |
| NC_045512.2_21mer_win1_29654 | 29654 | 29674 | 3312 | CAATTGAAATTAGAGTGTATC | 4294 | CTATGTGAGATTAAAGTTAAC |
| NC_045512.2_21mer_win1_29655 | 29655 | 29675 | 3313 | AATTGAAATTAGAGTGTATCG | 4295 | GCTATGTGAGATTAAAGTTAA |
| NC_045512.2_21mer_win1_29656 | 29656 | 29676 | 3314 | ATTGAAATTAGAGTGTATCGT | 4296 | TGCTATGTGAGATTAAAGTTA |
| NC_045512.2_21mer_win1_29657 | 29657 | 29677 | 3315 | TTGAAATTAGAGTGTATCGTT | 4297 | TTGCTATGTGAGATTAAAGTT |
| NC_045512.2_21mer_win1_29658 | 29658 | 29678 | 3316 | TGAAATTAGAGTGTATCGTTA | 4298 | ATTGCTATGTGAGATTAAAGT |
| NC_045512.2_21mer_win1_29659 | 29659 | 29679 | 3317 | GAAATTAGAGTGTATCGTTAG | 4299 | GATTGCTATGTGAGATTAAAG |
| NC_045512.2_21mer_win1_29660 | 29660 | 29680 | 3318 | AAATTAGAGTGTATCGTTAGA | 4300 | AGATTGCTATGTGAGATTAAA |
| NC_045512.2_21mer_win1_29661 | 29661 | 29681 | 3319 | AATTAGAGTGTATCGTTAGAA | 4301 | AAGATTGCTATGTGAGATTAA |
| NC_045512.2_21mer_win1_29662 | 29662 | 29682 | 3320 | ATTAGAGTGTATCGTTAGAAA | 4302 | AAAGATTGCTATGTGAGATTA |
| NC_045512.2_21mer_win1_29663 | 29663 | 29683 | 3321 | TTAGAGTGTATCGTTAGAAAT | 4303 | TAAAGATTGCTATGTGAGATT |
| NC_045512.2_21mer_win1_29664 | 29664 | 29684 | 3322 | TAGAGTGTATCGTTAGAAATT | 4304 | TTAAAGATTGCTATGTGAGAT |
| NC_045512.2_21mer_win1_29665 | 29665 | 29685 | 3323 | AGAGTGTATCGTTAGAAATTA | 4305 | ATTAAAGATTGCTATGTGAGA |
| NC_045512.2_21mer_win1_29666 | 29666 | 29686 | 3324 | GAGTGTATCGTTAGAAATTAG | 4306 | GATTAAAGATTGCTATGTGAG |
| NC_045512.2_21mer_win1_29667 | 29667 | 29687 | 3325 | AGTGTATCGTTAGAAATTAGT | 4307 | TGATTAAAGATTGCTATGTGA |
| NC_045512.2_21mer_win1_29689 | 29689 | 29709 | 3326 | ACACATTGTAATCCCTCCTGA | 4308 | AGTCCTCCCTAATGTTACACA |
| NC_045512.2_21mer_win1_29690 | 29690 | 29710 | 3327 | CACATTGTAATCCCTCCTGAA | 4309 | AAGTCCTCCCTAATGTTACAC |
| NC_045512.2_21mer_win1_29691 | 29691 | 29711 | 3328 | ACATTGTAATCCCTCCTGAAC | 4310 | CAAGTCCTCCCTAATGTTACA |
| NC_045512.2_21mer_win1_29692 | 29692 | 29712 | 3329 | CATTGTAATCCCTCCTGAACT | 4311 | TCAAGTCCTCCCTAATGTTAC |
| NC_045512.2_21mer_win1_29693 | 29693 | 29713 | 3330 | ATTGTAATCCCTCCTGAACTT | 4312 | TTCAAGTCCTCCCTAATGTTA |
| NC_045512.2_21mer_win1_29694 | 29694 | 29714 | 3331 | TTGTAATCCCTCCTGAACTTT | 4313 | TTTCAAGTCCTCCCTAATGTT |
| NC_045512.2_21mer_win1_29695 | 29695 | 29715 | 3332 | TGTAATCCCTCCTGAACTTTC | 4314 | CTTTCAAGTCCTCCCTAATGT |
| NC_045512.2_21mer_win1_29696 | 29696 | 29716 | 3333 | GTAATCCCTCCTGAACTTTCT | 4315 | TCTTTCAAGTCCTCCCTAATG |
| NC_045512.2_21mer_win1_29697 | 29697 | 29717 | 3334 | TAATCCCTCCTGAACTTTCTC | 4316 | CTCTTTCAAGTCCTCCCTAAT |
| NC_045512.2_21mer_win1_29698 | 29698 | 29718 | 3335 | AATCCCTCCTGAACTTTCTCG | 4317 | GCTCTTTCAAGTCCTCCCTAA |
| NC_045512.2_21mer_win1_29699 | 29699 | 29719 | 3336 | ATCCCTCCTGAACTTTCTCGG | 4318 | GGCTCTTTCAAGTCCTCCCTA |
| NC_045512.2_21mer_win1_29700 | 29700 | 29720 | 3337 | TCCCTCCTGAACTTTCTCGGT | 4319 | TGGCTCTTTCAAGTCCTCCCT |
| NC_045512.2_21mer_win1_29701 | 29701 | 29721 | 3338 | CCCTCCTGAACTTTCTCGGTG | 4320 | GTGGCTCTTTCAAGTCCTCCC |
| NC_045512.2_21mer_win1_29702 | 29702 | 29722 | 3339 | CCTCCTGAACTTTCTCGGTGG | 4321 | GGTGGCTCTTTCAAGTCCTCC |
| NC_045512.2_21mer_win1_29703 | 29703 | 29723 | 3340 | CTCCTGAACTTTCTCGGTGGT | 4322 | TGGTGGCTCTTTCAAGTCCTC |
| NC_045512.2_21mer_win1_29704 | 29704 | 29724 | 3341 | TCCTGAACTTTCTCGGTGGTG | 4323 | GTGGTGGCTCTTTCAAGTCCT |
| NC_045512.2_21mer_win1_29705 | 29705 | 29725 | 3342 | CCTGAACTTTCTCGGTGGTGT | 4324 | TGTGGTGGCTCTTTCAAGTCC |
| NC_045512.2_21mer_win1_29706 | 29706 | 29726 | 3343 | CTGAACTTTCTCGGTGGTGTA | 4325 | ATGTGGTGGCTCTTTCAAGTC |
| NC_045512.2_21mer_win1_29707 | 29707 | 29727 | 3344 | TGAACTTTCTCGGTGGTGTAA | 4326 | AATGTGGTGGCTCTTTCAAGT |
| NC_045512.2_21mer_win1_29708 | 29708 | 29728 | 3345 | GAACTTTCTCGGTGGTGTAAA | 4327 | AAATGTGGTGGCTCTTTCAAG |
| NC_045512.2_21mer_win1_29709 | 29709 | 29729 | 3346 | AACTTTCTCGGTGGTGTAAAA | 4328 | AAAATGTGGTGGCTCTTTCAA |
| NC_045512.2_21mer_win1_29710 | 29710 | 29730 | 3347 | ACTTTCTCGGTGGTGTAAAAG | 4329 | GAAAATGTGGTGGCTCTTTCA |
| NC_045512.2_21mer_win1_29711 | 29711 | 29731 | 3348 | CTTTCTCGGTGGTGTAAAAGT | 4330 | TGAAAATGTGGTGGCTCTTTC |
| NC_045512.2_21mer_win1_29733 | 29733 | 29753 | 3349 | GCTCCGGTGCGCCTCATGCTA | 4331 | ATCGTACTCCGCGTGGCCTCG |
| NC_045512.2_21mer_win1_29734 | 29734 | 29754 | 3350 | CTCCGGTGCGCCTCATGCTAG | 4332 | GATCGTACTCCGCGTGGCCTC |
| NC_045512.2_21mer_win1_29735 | 29735 | 29755 | 3351 | TCCGGTGCGCCTCATGCTAGC | 4333 | CGATCGTACTCCGCGTGGCCT |
| NC_045512.2_21mer_win1_29736 | 29736 | 29756 | 3352 | CCGGTGCGCCTCATGCTAGCT | 4334 | TCGATCGTACTCCGCGTGGCC |
| NC_045512.2_21mer_win1_29737 | 29737 | 29757 | 3353 | CGGTGCGCCTCATGCTAGCTC | 4335 | CTCGATCGTACTCCGCGTGGC |
| NC_045512.2_21mer_win1_29770 | 29770 | 29790 | 3354 | TTACGATCCCTCTCGACGGAT | 4336 | TAGGCAGCTCTCCCTAGCATT |
| NC_045512.2_21mer_win1_29771 | 29771 | 29791 | 3355 | TACGATCCCTCTCGACGGATA | 4337 | ATAGGCAGCTCTCCCTAGCAT |
| NC_045512.2_21mer_win1_29772 | 29772 | 29792 | 3356 | ACGATCCCTCTCGACGGATAT | 4338 | TATAGGCAGCTCTCCCTAGCA |
| NC_045512.2_21mer_win1_29773 | 29773 | 29793 | 3357 | CGATCCCTCTCGACGGATATA | 4339 | ATATAGGCAGCTCTCCCTAGC |
| NC_045512.2_21mer_win1_29774 | 29774 | 29794 | 3358 | GATCCCTCTCGACGGATATAC | 4340 | CATATAGGCAGCTCTCCCTAG |
| NC_045512.2_21mer_win1_29775 | 29775 | 29795 | 3359 | ATCCCTCTCGACGGATATACC | 4341 | CCATATAGGCAGCTCTCCCTA |
| NC_045512.2_21mer_win1_29776 | 29776 | 29796 | 3360 | TCCCTCTCGACGGATATACCT | 4342 | TCCATATAGGCAGCTCTCCCT |
| NC_045512.2_21mer_win1_29777 | 29777 | 29797 | 3361 | CCCTCTCGACGGATATACCTT | 4343 | TTCCATATAGGCAGCTCTCCC |
| NC_045512.2_21mer_win1_29778 | 29778 | 29798 | 3362 | CCTCTCGACGGATATACCTTC | 4344 | CTTCCATATAGGCAGCTCTCC |
| NC_045512.2_21mer_win1_29779 | 29779 | 29799 | 3363 | CTCTCGACGGATATACCTTCT | 4345 | TCTTCCATATAGGCAGCTCTC |
| NC_045512.2_21mer_win1_29780 | 29780 | 29800 | 3364 | TCTCGACGGATATACCTTCTC | 4346 | CTCTTCCATATAGGCAGCTCT |
| NC_045512.2_2lmer_win1_29781 | 29781 | 29801 | 3365 | CTCGACGGATATACCTTCTCG | 4347 | GCTCTTCCATATAGGCAGCTC |
| NC_045512.2_21mer_win1_29782 | 29782 | 29802 | 3366 | TCGACGGATATACCTTCTCGG | 4348 | GGCTCTTCCATATAGGCAGCT |
| NC_045512.2_21mer_win1_29783 | 29783 | 29803 | 3367 | CGACGGATATACCTTCTCGGG | 4349 | GGGCTCTTCCATATAGGCAGC |
| NC_045512.2_21mer_win1_29784 | 29784 | 29804 | 3368 | GACGGATATACCTTCTCGGGA | 4350 | AGGGCTCTTCCATATAGGCAG |
| NC_045512.2_21mer_win1_29785 | 29785 | 29805 | 3369 | ACGGATATACCTTCTCGGGAT | 4351 | TAGGGCTCTTCCATATAGGCA |
| NC_045512.2_21mer_win1_29786 | 29786 | 29806 | 3370 | CGGATATACCTTCTCGGGATT | 4352 | TTAGGGCTCTTCCATATAGGC |
| NC_045512.2_21mer_win1_29787 | 29787 | 29807 | 3371 | GGATATACCTTCTCGGGATTA | 4353 | ATTAGGGCTCTTCCATATAGG |
| NC_045512.2_21mer_win1_29788 | 29788 | 29808 | 3372 | GATATACCTTCTCGGGATTAC | 4354 | CATTAGGGCTCTTCCATATAG |
| NC_045512.2_21mer_win1_29789 | 29789 | 29809 | 3373 | ATATACCTTCTCGGGATTACA | 4355 | ACATTAGGGCTCTTCCATATA |
| NC_045512.2_21mer_win1_29790 | 29790 | 29810 | 3374 | TATACCTTCTCGGGATTACAC | 4356 | CACATTAGGGCTCTTCCATAT |
| NC_045512.2_2lmer_win1_29791 | 29791 | 29811 | 3375 | ATACCTTCTCGGGATTACACA | 4357 | ACACATTAGGGCTCTTCCATA |
| NC_045512.2_21mer_win1_29792 | 29792 | 29812 | 3376 | TACCTTCTCGGGATTACACAT | 4358 | TACACATTAGGGCTCTTCCAT |
| NC_045512.2_21mer_win1_29793 | 29793 | 29813 | 3377 | ACCTTCTCGGGATTACACATT | 4359 | TTACACATTAGGGCTCTTCCA |
| NC_045512.2_21mer_win1_29794 | 29794 | 29814 | 3378 | CCTTCTCGGGATTACACATTT | 4360 | TTTACACATTAGGGCTCTTCC |
| NC_045512.2_21mer_win1_29795 | 29795 | 29815 | 3379 | CTTCTCGGGATTACACATTTT | 4361 | TTTTACACATTAGGGCTCTTC |
| NC_045512.2_21mer_win1_29796 | 29796 | 29816 | 3380 | TTCTCGGGATTACACATTTTA | 4362 | ATTTTACACATTAGGGCTCTT |
| NC_045512.2_21mer_win1_29797 | 29797 | 29817 | 3381 | TCTCGGGATTACACATTTTAA | 4363 | AATTTTACACATTAGGGCTCT |
| NC_045512.2_21mer_win1_29798 | 29798 | 29818 | 3382 | CTCGGGATTACACATTTTAAT | 4364 | TAATTTTACACATTAGGGCTC |
| NC_045512.2_21mer_win1_29799 | 29799 | 29819 | 3383 | TCGGGATTACACATTTTAATT | 4365 | TTAATTTTACACATTAGGGCT |
| NC_045512.2_21mer_win1_29800 | 29800 | 29820 | 3384 | CGGGATTACACATTTTAATTA | 4366 | ATTAATTTTACACATTAGGGC |
| NC_045512.2_21mer_win1_29801 | 29801 | 29821 | 3385 | GGGATTACACATTTTAATTAA | 4367 | AATTAATTTTACACATTAGGG |
| NC_045512.2_21mer_win1_29802 | 29802 | 29822 | 3386 | GGATTACACATTTTAATTAAA | 4368 | AAATTAATTTTACACATTAGG |
| NC_045512.2_21mer_win1_29803 | 29803 | 29823 | 3387 | GATTACACATTTTAATTAAAA | 4369 | AAAATTAATTTTACACATTAG |
| NC_045512.2_21mer_win1_29804 | 29804 | 29824 | 3388 | ATTACACATTTTAATTAAAAT | 4370 | TAAAATTAATTTTACACATTA |
| NC_045512.2_21mer_win1_29805 | 29805 | 29825 | 3389 | TTACACATTTTAATTAAAATC | 4371 | CTAAAATTAATTTTACACATT |
| NC_045512.2_21mer_win1_29806 | 29806 | 29826 | 3390 | TACACATTTTAATTAAAATCA | 4372 | ACTAAAATTAATTTTACACAT |
| NC_045512.2_21mer_win1_29807 | 29807 | 29827 | 3391 | ACACATTTTAATTAAAATCAT | 4373 | TACTAAAATTAATTTTACACA |
| NC_045512.2_21mer_win1_29808 | 29808 | 29828 | 3392 | CACATTTTAATTAAAATCATC | 4374 | CTACTAAAATTAATTTTACAC |

TABLE 2
Genome Sequences for Coronaviruses

| SEQ | | |
|---|---|---|
| ID | Des- | |
| NO | cription | Sequence |
| 2407 | SARS-CoV- | attaaaggtttataccttcccaggtaacaaaccaaccaactttcgatctcttgtagatctgttctctaaacgaactttaaaatctgtgtggctgtcactcggctgcatgcttagtgc |
| | 2 genome | actcacgcagtataattaataactaattactgtcgttgacaggacacgagtaactcgtctatcttctgcaggctgcttacggtttcgtccgtgttgcagccgatcatcagcacat |
| | (Genbank | ctaggtttcgtccgggtgtgaccgaaaggtaagatggagagccttgtccctggtttcaacgagaaaacacacgtccaactcagtttgcctgttttacaggttcgcgacgtgct |
| | Accession | cgtacgtggctttggagactccgtggaggaggtatatcagaggcacgtcaacatcttaaagatggcacttgtggcttagtagaagttgaaaaaggcgttttgcctcaacttg |
| | No. | aacagccctatgtgttcatcaaacgttcggatgctcgaactgcacctcatggtcatgttatggttgagctggtagcagaactcgaaggcattcagtacggtcgtagtggtgag |
| | NC_ | acacttggtgtccttgtccctcatgtgggcgaaataccagtggcttaccgcaaggttcttcttcgtaagaacggtaataaaggagctggtggccatagttacggcgccgatct |
| | 045512.2) | aaagtcatttgacttaggcgacgagcttggcactgatccttatgaagattttcaagaaaactggaacactaaacatagcagtggtgttacccgtgaactcatgcgtgagctta |
| | | acggaggggcatacactcgctatgtcgataacaacttctgtggccctgatggctaccctcttgagtgcattaaagaccttctagcacgtgctggtaaagcttcatgcactttgt |
| | | ccgaacaactggactttattgacactaagaggggtgtatactgctgccgtgaacatgagcatgaaattgcttggtacacggaacgttctgaaaagagctatgaattgcagac |
| | | acatttgaaattaaattggcaaagaaatttgacaccttcaatggggaatgtccaaattttgtatttcccttaaattccataatcaagactattcaaccaagggttgaaaagaaaa |
| | | agcttgatggctttatgggtagaattcgatctgtctatccagttgcgtcaccaaatgaatgcaaccaaatgtgcctttcaactctcatgaagtgtgatcattgtggtgaaacttca |
| | | tggcagacgggcgattttgttaaagccacttgcgaattttgtggcactgagaatttgactaaagaaggtgccactacttgtggttacttaccccaaaatgctgttgttaaaattta |
| | | ttgtccagcatgtcacaattcagaagtaggacctgagcatagtcttgccgaataccataatgaatctggcttgaaaaccattcttcgtaagggtggtcgcactattgcctttgga |
| | | ggctgtgtgttctcttatgttggttgccataacaagtgtgcctattgggttccacgtgctagcgctaacataggttgtaaccatacaggtgttgttggagaaggttccgaaggtc |
| | | ttaatgacaaccttcttgaaatactccaaaaagagaaagtcaacatcaatattgttggtgactttaaacttaatgaagagatcgccattattttggcatctttttctgcttccacaag |
| | | tgatttgtggaaactgtgaaaggtttggattataaagcattcaaacaaattgttgaatcctgtggtaattttaaagttacaaaaggaaaagctaaaaaaggtgcctggaatattg |
| | | gtgaacagaaatcaatactgagtcctattatgcatttgcatcagaggctgctcgtgttgtacgatcaattttctcccgcactcttgaaactgctcaaaattctgtgcgtgttttaca |
| | | gaaggccgctataacaatactagatggaatttcacagtattcactgagactcattgatgctatgatgttcacatctgatttggctactaacaatctagttgtaatggcctacattac |
| | | aggtggtgttgttcagttgacttcgcagtggctaactaacatattggcactgtttatgaaaaactcaaacccgtccttgattggcttgaagagaagtttaaggaaggtgtagag |
| | | tttcttagagacggttgggaaattgttaaatttatctcaacctgtgcttgtgaaattgtcggtggacaaattgtcacctgtgcaaaggaaattaaggagagtgttcagacattcttt |
| | | aagcttgtaaataaatttttggctttgtgtgctgactctatcattattggtggagctaaacttaaagccttgaatttaggtgaaacatttgtcacgcactcaaagggattgtacaga |
| | | aagtgtgttaaatccagagaagaaactggcctactcatgcctctaaaagccccaaaagaaattatcttcttagagggagaaacacttcccacagaagtgttaacagaggaa |
| | | gttgtcttgaaaactggtgatttacaaccattagaacaacctactagtgaagctgttgaagctccattggttggtacaccagtttgtattaacgggcttatgttgctcgaaatcaa |
| | | agacacagaaaagtactgtgccatgcacctaatatgatggtaacaaacaataccttcacactcaaaggcggtgcaccaacaaaggttacttttggtgatgacactgtgata |
| | | gaagtgcaaggttacaagagtgtgaatatcacttttgaacttgatgaaaggattgataaagtacttaatgagaagtgctctgcctatacagttgaactcggtacagaagtaaat |
| | | gagttcgcctgtgttgtggcagatgctgtcataaaaactttgcaaccagtatctgaattacttacaccactgggcattgatttagatgagtggagtatggctacatactacttattt |
| | | gatgagtaggtgagtttaaattggcttcacatatgtattgttattctaccctccagatgaggatgaagaagaaggtgattgtgaagaagaagagtttgagccatcaactcaat |
| | | atgagtatggtactgaagatgattaccaaggtaaacctttggaatttggtgccacttctgctgacttcaacctgaagaagagcaagaagaagattggttagatgatgatagtc |
| | | aacaaactgttggtcaacaagacggcagtgaggacaatcagacaactactattcaaacaattgttgaggttcaacctcaattagagatggaacttacaccagttgttcagact |
| | | attgaagtgaatagttttagtggttatttaaaacttactgacaatgtatacattaaaaatgcagacattgtggaagaagctaaaaaggtaaaaccaacagtggttgttaatgcag |
| | | ccaatgtttaccttaaacatggaggaggtgttgcaggagccttaaataaggctactaacaatgccatgcaagttgaatctgatgattacatagctactaatggaccacttaaag |
| | | tgggtggtagttgtgttttaagcggacacaatcttgctaaacactgtcttcatgttgtcggcccaaatgttaacaaaggtgaagacattcaacttcttaagagtgcttatgaaaat |
| | | tttaatcagcacgaagttctacttgcaccattattatcagaggtatttttggtgctgaccctatacattattaagagtttgtgtagatactgttcgcacaaatgtctacttagctgtc |
| | | tttgataaaaatctctatgacaaacttgtttcaagattttggaaatgaagagtgaaaagcaagttgaacaaaagatcgctgagattcctaaagaggaagttaagccatttataa |
| | | ctgaaagtaaaccttcagttgaacagagaaaacaagatgataagaaaatcaaagcttgtgttgaagaagttacaacaactctggaagaaactaagttcctcacagaaaactt |
| | | gttactttatattgacattaatggcaatcttcatccagattctgccactcttgttagtgacattgacatcactttcttaaagaaagatgctccatatatagtgggtgatgttgttcaag |
| | | agggtgttttaactgctgtggttatacctactaaaaaggctggtggcactactgaaatgctagcgaaagctttgagaaaagtgccaacagacaattatataaccacttacccg |
| | | ggtcagggtttaaatggttacactgtagaggaggcaaagacagtgcttaaaaagtgtaaaagtgccttttacattctaccatctattatctctaatgagaagcaagaaattcttg |
| | | gaactgtttcttggaatttgcgagaaatgcttgcacatgcagaagaaacacgcaaattaatgcctgtagtgtggaaactaaagccatagtttcaactatacagcgtaaatata |
| | | agggtattaaaatacaagagggtgtggttgattatggtgctagattttacttttacaccagtaaaacaactgtagcgtcacttatcaacacacttaacgatctaaatgaaactctt |
| | | gttacaatgccacttggctatgtaacacatggcttaaatttggaagaagctgctcggtatatgagatctctcaaagtgccagctacagtttctgtttcttcacctgatgctgttaca |
| | | gcgtataatggttatcttacttcttcttctaaaacacctgaagaacattttattgaaaccatctcacttgctggttcctataaagattggtcctattctggacaatctacacaactagg |
| | | tatagaatttcttaagagaggtgataaaagtgtatattacactagtaatcctaccacattccacctagatggtgaagttatcacctttgacaatcttaagacacttattctttgaga |
| | | gaagtgaggactattaaggtgtttacaacagtagacaacattaacctccacacgcaagttgtggacatgtcaatgacatatggacaacagtttggtccaacttatttggatgg |
| | | agctgatgttactaaaataaaacctcataattcacatgaaggtaaaacattttatgttttacctaatgatgacactctacgtgttgaggcttttgagtactaccacacaactgatcct |
| | | agttttctgggtaggtacatgtcagcattaaatcacactaaaaagtggaaatacccacaagttaatggtttaacttctattaaatgggcagataacaactgttatcttgccactgc |
| | | attgttaacactccaacaaatagagttgaagtttaatccacctgctctacaagatgcttattacagagcaagggctggtgaagctgctaacttttgtgcacttatcttagcctact |
| | | gtaataagacagtaggtgagttaggtgatgttagagaaacaatgagttacttgtttcaacatgccaatttagattcttgcaaaagagtcttgaacgtggtgtgtaaaacttgtgg |
| | | acaacagcagacaacccttaagggtgtagaagctgttatgtacatgggcacactttcttatgaacaatttaagaaaggtgttcagataccttgtacgtgtggtaaacaagctac |
| | | aaaatatctagtacaacaggagtcaccttttgttatgatgtcagcaccacctgctcagtatgaacttaagcatggtacatttacttgtgctagtgagtacactggtaattaccagt |
| | | gtggtcactataaacatataacttctaaagaaactttgtattgcatagacggtgattacttacaaagtcctcagaatacaaaggtcctattacggatgttttctacaaagaaaac |
| | | agttacacaacaaccataaaaccagttacttataaattggatggtgttgtttgtacagaaattgaccctaagttggacaattattataagaaagacaattcttatttcacagagca |
| | | accaattgatcttgtaccaaaccaaccatatccaaacgcaagcttcgataattttaagtttgtatgtgataatatcaaatttgctgatgatttaaaccagttaactggttataagaaa |
| | | cctgcttcaagagagcttaaagttacatttttccctgacttaaatggtgatgtggtggctattgattataaacactacacaccctatttaagaaaggagctaaattgttacataaa |
| | | cctattgtttggcatgttaacaatgcaactaataaagccacgtataaaccaaatacctggtgtatacgttgtctttggagcacaaaaccagttgaaacatcaaattcgtttgatgt |
| | | actgaagtcagaggacgcgcagggaatggataatcttgcctgcgaagatctaaaaccagtactgaagaagtagtggaaaatcctaccatacagaaagacgttcttgagt |
| | | gtaatgtgaaaactaccgaagttgtaggagacattatacttaaaccagcaaataatagtttaaaaattacagaagaggttggccacacagatctaatggctgcttatgtagac |
| | | aattctagtcttactattaagaaacctaatgaattatctagagtattaggtttgaaaacccttgctactcatggtttagctgctgttaatagtgtcccttgggatactatagctaattat |
| | | gctaagccttttcttaacaaagttgttagtacaactactaacatagttacacggtgtttaaaccgtgtttgtactaattatatgccttatttattactttattgctacaattgtgtactttt |
| | | actagaagtacaaattctagaattaaagcatctatgccgactactatagcaaagaatactgttaagagtgtcggtaaattttgtctagaggcttcatttaattatttgaagtcacct |
| | | aatttttctaaactgataaatattataatttggtttttactattaagtgtttgcctaggttctttaatctactcaaccgctgctttaggtgttttaatgtctaatttaggcatgccttcttact |
| | | gtactggttacagagaaggctatttgaactctactaatgtcactattgcaacctactgtactggttctataccttgtagtgtttgtcttagtggtttagattattagacacctatcctt |
| | | ctttagaaactatacaaattaccatttcatcttttaaatgggatttaactgatttggcttagttgcagagtggtttttggcatatattatttcactaggtttttctatgtacttggattgg |
| | | ctgcaatcatgcaattgtttttcagctattttgcagtacattttattagtaattcttggcttatgtggttaataattaatcttgtacaaatggccccgatttcagctatggttagaatgta |
| | | catcttctttgcatcattttattatgtatggaaaagttatgtgcatgttgtagacggttgtaattcatcaacttgtatgatgtgttacaaacgtaatagagcaacaagagtcgaatgt |
| | | acaactattgttaatggtgttagaaggtccttttatgtctatgctaatggaggtaaaggcttttgcaaactacacaattggaattgtgttaattgtgatacattctgtgctggtagta |
| | | catttattagtgatgaagttgcgagagacttgtcactacagtttaaaagaccaataaatcctactgaccagtcttcttacatcgttgatagtgttacagtgaagaatggttccatcc |
| | | atctttactttgataaagctggtcaaaagacttatgaaagacattctctctctcattttgttaacttagacaacctgagagctaataacactaaaggttcattgcctattaatgttata |
| | | gtttttgatggtaaatcaaaatgtgaagaatcatctgcaaaatcagcgtctgtttactacagtcagcttatgtgtcaacctatactgttactagatcaggcattagtgtctgatgttg |
| | | gtgatagtgcggaagttgcagttaaaatgtttgatgcttacgttaatacgttttcatcaacttttaacgtaccaatggaaaaactcaaaacactagttgcaactgcagaagctga |
| | | acttgcaaagaatgtgtccttagacaatgtcttatctacttttatttcagcagctcggcaagggtttgttgattcagatgtagaaactaaagatgttgttgaatgtcttaaattgtca |
| | | catcaatctgacatagaagttactggcgatagttgtaataactatatgctcacctataacaaagttgaaaacatgacaccccgtgaccttggtgcttgtattgactgtagtgcgc |
| | | gtcatattaatgcgcaggtagcaaaaagtcacaacattgctttgatatggaacgttaaagatttcatgtcattgtctgaacaactacgaaaacaaatacgtagtgctgctaaaa |
| | | agaataacttaccttttaagttgacatgtgcaactactagacaagttgttaatgttgtaacaacaaagatagcacttaagggtggtaaaattgttaataattggttgaagcagtta |
| | | attaaagttacacttgtgttcctttttgttgctgctattttctatttaataacacctgttcatgtcatgtctaaacatactgacttttcaagtgaaatcataggatacaaggctattgatg |
| | | gtggtgtcactcgtgacatagcatctacagatacttgttttgctaacaaacatgctgattttgacacatggtttagccagcgtggtggtagttatactaatgacaaagcttgccca |
| | | ttgattgctgcagtcataacaagagaagtgggttttgtcgtgcctggtttgcctggcacgatattacgcacaactaatggtgactttttgcatttcttacctagagtttttagtgca |
| | | gttggtaacatctgttacacaccatcaaaacttatagagtacactgactttgcaacatcagcttgtgttttggctgctgaatgtacaatttttaaagatgcttctggtaagccagta |
| | | ccatattgttatgataccaatgtactagaaggttctgttgcttatgaaagtttacgccctgacacacgttatgtgctcatggatggctctattattcaatttcctaacacctaccttga |
| | | aggttctgttagagtggtaacaacttttgattctgagtactgtaggcacggcacttgtgaaagatcagaagctggtgtttgtgtatctactagtggtagatgggtacttaacaat |
| | | gattattacagatattaccaggagttttctgtggtgtagatgctgtaaatttacttactaatatgtttacaccactaattcaacctattggtgattggacatatcagcatctatagta |
| | | gctggtggtattgtagctatcgtagtaacatgccttgcctactattttatgaggtttagaagagcttttggtgaatacagtcatgtagttgcctttaatactttactattccttatgtcat |
| | | tcactgtactagtttaacaccagtttactcattcttacctggtgtttattctgttatttacttgtacttgacattttatcttactaatgatgtttcttttttagcacatattcagtggatggtt |
| | | atgttcacacctttagtacctttctggataacaattgcttatatcatttgtatttccacaaagcatttctattggttctttagtaattacctaaagagacgtgtagtattaatggtgtttcct |
| | | ttagtacttttgaagaagctgcgctgtgcacctttttgttaaataaagaaatgtatctaaagttgcgtagtgatgtgctattacctcttacgcaatataatagatacttagctctttata |
| | | ataagtacaagtattttagtggagcaatggatacaactagctacagagaagctgcttgttgtcatctcgcaaaggctctcaatgacttcagtaactcaggttctgatgttctttac |
| | | caaccaccacaaacctctatcacctcagctgttttgcagagtggttttagaaaaatggcattcccatctggtaaagttgagggttgtatggtacaagtaacttgtggtacaacta |
| | | cacttaacggtattggcttgatgacgtagtttactgtccaagacatgtgatctgcacctctgaagacatgcttaaccctaattatgaagatttactcattcgtaagtctaatcataa |
| | | tttcttggtacaggctggtaatgttcaactcagggttattggacattctatgcaaaattgtgtacttaagcttaaggttgatacagccaatcctaagacacctaagtataagtttgtt |
| | | cgcattcaaccaggacagactttttcagtgttagcttgttacaatggttcaccatctggtgtttaccaatgtgctatgaggcccaatttcactattaagggttcattccttaatggtt |
| | | catgtggtagtgttggttttaacatagattatgactgtgtctattttgttacatgcaccatatggaattaccaactggagttcatgctggcacagacttagaaggtaacttttatgg |
| | | accttttgttgacaggcaaacagcacaagcagctggtacggacacaactattacagttaatgttttagcttggttgtacgctgctgttataaatggagacaggtggtttctcaat |
| | | cgatttaccacaactcttaatgactttaaccttgtggctatgaagtacaattatgaacctctaacacaagaccatgttgacatactaggacctattctgctcaaactggaattgc |
| | | cgttttagatatgtgtgcttcattaaaagaattactgcaaaatggtatgaatggacgtaccatattgggtagtgattattagaagatgaatttacaccttttgatgttgttagacaat |
| | | gctcaggtgttactttccaaagtgcagtgaaaagaacaatcaagggtacacaccactggttgttactcacaattttgacttcacttttagttttagtccagagtactcaatggtctt |
| | | tgttatttttttgtatgaaaatgcctttttaccttttgctatgggtattattgctatgtctgatttgcaatgatgtttgtcaaacataagcatgcatttactgtttgtttttgttaccttctct |
| | | tgccactgtagcttattttaatatggtctatatgcctgctagttgggtgatgcgtattatgacatggttggatatggttgatactagtttgtaggttttaagctaaaagactgtgttat |
| | | gtatgcatcagctgtagtgttactaatccttatgacagcaagaactgtgtatgatgatggtgctaggagagtgtggacacttatgaatgtcttgacactcgtttataaagtttatta |
| | | tggtaatgattagatcaagccatttccatgtgggctcttataatctctgttacttctaactactcaggtgtagttacaactgtcatgtttttggccagaggtattgtttttatgtgtgtt |
| | | gagtattgccctattttcttcataactggtaatacacttcagtgtataatgctagtttattgtttcttaggctatttttgtacttgttactttggcctatttgtttactcaaccgctactttag |
| | | actgactcttggtgtttatgattacttagtttctacacaggagtttagatatatgaattcacagggactactcccacccaagaatagcatagatgccttcaaactcaacattaaatt |
| | | gttgggtgttggtggcaaaccttgtatcaaagtagccactgtacagtctaaaatgtcagatgtaaagtgcacatcagtagtcttactctcagttttgcaacaactcagagtagaa |
| | | tcatcatctaaattgtgggctcaatgtgtccagttacacaatgacattctcttagctaaagatactactgaagcctttgaaaaaatggtttcactactttctgttttgattccatgca |
| | | gggtgctgtagacataaacaagattgtgaagaaatgctggacaacagggcaaccttacaagctatagcctcagagtttagttccdtccatcatatgcagcttttgctactgc |
| | | tcaagaagcttatgagcaggctgttgctaatggtgattctgaagttgttcttaaaaagttgaagaagtattgaatgtggctaaatctgaatttgaccgtgatgcagccatgcaa |
| | | cgtaagttggaaaagatggctgatcaagctatgacccaaatgtataaacaggctagatctgaggacaagagggcaaaagttactagtgctatgcagacaatgatttcacta |
| | | tgcttagaaagttggataatgatgcactcaacaacattatcaacaatgcaagagatggttgtgttccdtgaacataatacctcttacaacagcagccaaactaatggttgtcat |
| | | accagactataacacatataaaaatacgtgtgatggtacaacatttacttatgcatcagcattgtgggaaatccaacaggttgtagatgcagatagtaaaattgttcaacttagt |
| | | gaaattagtatggacaattcacctaatttagcatggcctcttattgtaacagctttaagggccaattctgctgtcaaattacagaataatgagcttagtcctgttgcactacgaca |
| | | gatgtcttgtgctgccggtactacacaaactgcttgcactgatgacaatgcgttagcttactacaacacaacaaagggaggtaggtttgtacttgcactgttatccgatttacag |
| | | gatttgaaatgggctagattccctaagagtgatggaactggtactatctatacagaactggaaccaccttgtaggtttgttacagacacacctaaaggtcctaaagtgaagtat |
| | | ttatactttattaaaggattaaacaacctaaatagaggtatggtacttggtagtttagctgccacagtacgtctacaagctggtaatgcaacagaagtgcctgccaattcaactg |
| | | tattatattctgtgatttgctgtagatgctgctaaagcttacaaagattatctagctagtgggggacaaccaatcactaattgtgttaagatgttgtgtacacacactggtactg |
| | | gtcaggcaataacagttacaccggaagccaatatggatcaagaatcctttggtggtgcatcgtgttgtctgtactgccgttgccacatagatcatccaaatcctaaaggatttt |
| | | gtgacttaaaaggtaagtatgtacaaatacctacaacttgtgctaatgaccctgtgggttttacacttaaaaacacagtagtaccgtctgcggtatgtggaaaggttatggctg |
| | | tagttgtgatcaactccgcgaacccatgcttcagtcagctgatgcacaatcgtttttaaacgggtttgcggtgtaagtgcagcccgtcttacaccgtgcggcacaggcactag |
| | | tactgatgtcgtatacagggcttttgacatctacaatgataaagtagctggttttgctaaattcctaaaaactaattgttgtcgcttccaagaaaaggacgaagatgacaatttaat |
| | | tgattcttactttgtagttaagagacacactttctctaactaccaacatgaagaaacaatttataatttacttaaggattgtccagagttgctaaacatgacttattaagtttagaat |
| | | agacggtgacatggtaccacatatatcacgtcaacgtcttactaaatacacaatggcagacctcgtctatgattaaggcattttgatgaaggtaattgtgacacattaaaaga |
| | | aatacttgtcacatacaattgttgtgatgatgattatttcaataaaaaggactggtatgattttgtagaaaacccagatatattacgcgtatacgccaacttaggtgaacgtgtac |
| | | gccaagattgttaaaaacagtacaattctgtgatgccatgcgaaatgctggtattgttggtgtactgacattagataatcaagatctcaatggtaactggtatgatttcggtgatt |
| | | tcatacaaaccacgccaggtagtggagttcctgttgtagattcttattattcattgttaatgcctatattaaccttgaccagggctttaactgcagagtcacatgttgacactgactt |
| | | aacaaagccttacattaagtgggatttgttaaaatatgacttcacggaagagaggttaaaactattgaccgttattttaaatattgggatcagacataccacccaaattgtgtta |
| | | actgtttggatgacagatgcattctgcattgtgcaaactttaatgttttattctctacagtgttcccacctacaagttttggaccactagtgagaaaaatatttgttgatggtgttcca |
| | | tttgtagtttcaactggataccacttcagagagctaggtgttgtacataatcaggatgtaaacttacatagctctagacttagttttaaggaattacttgtgtatgctgctgaccctg |
| | | ctatgcacgctgcttctggtaatctattactagataaacgcactacgtgatttcagtagctgcacttactaacaatgttgatttcaaactgtcaaacccggtaattttaacaaag |
| | | acttctatgactttgctgtgtctaagggtttctttaaggaaggaagttctgttgaattaaaacacttcttctttgctcaggatggtaatgctgctatcagcgattatgactactatcgt |
| | | tataatctaccaacaatgtgtgatatcagacaactactatttgtagttgaagttgttgataagtactttgattgttacgatggtggctgtattaatgctaaccaagtcatcgtcaaca |
| | | acctagacaaatcagctggttttccatttaataaatggggtaaggctagactttattatgattcaatgagttatgaggatcaagatgcacttttcgcatatacaaaacgtaatgtca |
| | | tccctactataactcaaatgaatcttaagtatgccattagtgcaaagaatagagctcgcaccgtagctggtgtctctatctgtagtactatgaccaatagacagtttcatcaaaa |
| | | attattgaaatcaatagccgccactagaggagctactgtagtaattggaacaagcaaattctatggtggttggcacaacatgttaaaaactgtttatagtgatgtagaaaaccc |
| | | tcaccttatgggttgggattatcctaaatgtgatagagccatgcctaacatgcttagaattatggcctcacttgttcttgctcgcaaacatacaacgtgttgtagcttgtcacacc |
| | | gtttctatagattagctaatgagtgtgctcaagtattgagtgaaatggtcatgtgtggcggttcactatatgttaaaccaggtggaacctcatcaggagatgccacaactgctta |
| | | tgctaatagtgtttttaacatttgtcaagctgtcacggccaatgttaatgcacttttatctactgatggtaacaaaattgccgataagtatgtccgcaatttacaacacagactttat |
| | | gagtgtctctatagaaatagagatgttgacacagactttgtgaatgagttttacgcatatttgcgtaaacatttctcaatgatgatactctctgacgatgctgttgtgtgtttcaata |
| | | gcacttatgcatctcaaggtctagtggctagcataaagaactttaagtcagttctttattatcaaaacaatgtttttatgtctgaagcaaaatgttggactgagactgaccttacta |
| | | aaggacctcatgaattttgctctcaacatacaatgctagttaaacagggtgatgattatgtgtaccttccttacccagatccatcaagaatcctaggggccggctgttttgtagat |
| | | gatatcgtaaaaacagatggtacacttatgattgaacggttcgtgtattagctatagatgcttacccacttactaaacatcctaatcaggagtatgctgatgtattcatttgtactt |
| | | acaatacataagaaagctacatgatgagttaacaggacacatgttagacatgtattctgttatgcttactaatgataacacttcaaggtattgggaacctgagt-tttatgaggcta |
| | | tgtacacaccgcatacagtcttacaggctgttggggcttgtgttctttgcaattcacagacttcattaagatgtggtgcttgcatacgtagaccattcttatgttgtaaatgctgtta |
| | | cgaccatgtcatatcaacatcacataaattagtcttgtagttaatccgtatgtttgcaatgaccaggttgtgatgtcacagatgtgactcaactttacttaggaggtatgagctat |
| | | tattgtaaatcacataaaccacccattagttttccattgtgtgctaatggacaagtttttggtttatataaaaatacatgtgttggtagcgataatgttactgactttaatgcaattgca |
| | | acatgtgactggacaaatgctggtgattacattttagctaacacctgtactgaaagactcaagctttttgcagcagaaacgctcaaagctactgaggagacatttaaactgtctt |
| | | atggtattgctactgtacgtgaagtgctgtctgacagagaattacatctttcatgggaagttggtaaacctagaccaccacttaaccgaaattatgtctttactggttatcgtgta |
| | | actaaaaacagtaaagtacaaataggagagtacacctttgaaaaaggtgactatggtgatgctgttgtttaccgaggtacaacaacttacaaattaaatgttggtgattattttgt |
| | | gctgacatcacatacagtaatgccattaagtgcacctacactagtgccacaagagcactatgttagaattactggcttatacccaacactcaatatctcagatgagttttctagc |
| | | aatgttgcaaattatcaaaaggttggtatgcaaaagtattctacactccagggaccacctggtactggtaagagtcattttgctattggcctagctctctactacccttctgctcg |
| | | catagtgtatacagcttgactcatgccgctgttgatgcactatgtgagaaggcattaaaatatttgcctatagataaatgtagtagaattatacctgcacgtgctcgtgtagagt |
| | | gttttgataaattcaaagtgaattcaacattagaacagtatgtatttgtactgtaaatgcattgcctgagacgacagcagatatagttgtattgatgaaatttcaatggccacaa |
| | | attatgatttgagtgttgtcaatgccagattacgtgctaagcactatgtgtacattggcgaccctgctcaattacctgcaccacgcacattgctaactaagggcacactagaac |
| | | cagaatatttcaattcagtgtgtagacttatgaaaactataggtccagacatgttcctcggaacttgtcggcgttgtcctgctgaaattgttgacactgtgagtgctttggtttatg |
| | | ataataagcttaaagcacataaagacaaatcagctcaatgctttaaaatgttttataagggtgttatcacgcatgatgtttcatctgcaattaacaggccacaaataggcgtggt |
| | | aagagaattccttacacgtaaccctgcttggagaaaagagtctttatttcaccttataattcacagaatgctgtagcctcaaagattttgggactaccaactcaaactgttgattc |
| | | atcacagggctcagaatatgactatgtcatattcactcaaaccactgaaacagctcactcttgtaatgtaaacagatttaatgttgctattaccagagcaaaagtaggcatacttt |
| | | gcataatgtctgatagagacctttatgacaagttgcaatttacaagtcttgaaattccacgtaggaatgtggcaactttacaagctgaaaatgtaacaggactctttaaagattgt |
| | | agtaaggtaatcactgggttacatcctacacaggcacctacacacctcagtgttgacactaaattcaaaactgaaggtttatgtgttgacatacctggcatacctaaggacatg |
| | | acctatagaagactcatctctatgatgggttttaaaatgaattatcaagttaatggttaccctaacatgtttatcacccgcgaagaagctataagacatgtacgtgcatggattgg |
| | | cttcgatgtcgaggggtgtcatgctactagagaagctgttggtaccaatttacctttacagctaggtttttctacaggtgttaacctagttgctgtacctacaggttatgttgatac |
| | | acctaataatacagatttttccagagttagtgctaaaccaccgcctggagatcaatttaaacacctcataccacttatgtacaaaggacttccttggaatgtagtgcgtataaag |
| | | attgtacaaatgttaagtgacacacttaaaaatctactgacagagtcgtatttgtcttatgggcacatggctttgagttgacatctatgaagtattttgtgaaaataggacctgag |
| | | cgcacctgttgtctatgtgatagacgtgccacatgcttttccactgcttcagacacttatgcctgttggcatcattctattggatttgattacgtctataatccgtttatgattgatgtt |
| | | caacaatggggttttacaggtaacctacaaagcaaccatgatctgtattgtcaagtccatggtaatgcacatgtagctagttgtgatgcaatcatgactaggtgtctagctgtcc |
| | | acgagtgattgttaagcgtgttgactggactattgaatatcctataattggtgatgaactgaagattaatgcggcttgtagaaaggttcaacacatggttgttaaagctgcatta |
| | | ttagcagacaaattcccagttcttcacgacattggtaaccctaaagctattaagtgtgtacctcaagctgatgtagaatggaagttctatgatgcacagccttgtagtgacaaa |
| | | gcttataaaatagaagaattattctattcttatgccacacattctgacaaattcacagatggtgtatgcctattttggaattgcaatgtcgatagatatcctgctaattccattgtttgt |
| | | agatttgacactagagtgctatctaaccttaacttgcctggttgtgatggtggcagtttgtatgtaaataaacatgcattccacacaccagcttttgataaaagtgatttgttaattt |
| | | aaaacaattaccatttttctattactctgacagtccatgtgagtctcatggaaaacaagtagtgtcagatatagattatgtaccactaaagtagctacgtgtataacacgttgcaa |
| | | tttaggtggtgctgtctgtagacatcatgctaatgagtacagattgtatctcgatgcttataacatgatgatctcagctggctttagcttgtgggtttacaaacaatttgatacttata |
| | | acctaggaacacttttacaagacttcagagtttagaaaatgtggcttttaatgttgtaaataagggacactttgatggacaacagggtgaagtaccagtttctatcattaataac |
| | | actgtttacacaaaagttgatggtgttgatgtagaattgtttgaaaataaaacaacattacctgttaatgtagcatttgagctttgggctaagcgcaacattaaaccagtaccaga |
| | | ggtgaaaatactcaataatttgggtgtggacattgctgctaatactgtgatctgggactacaaaagagatgctccagcacatatatctactattggtgtttgttctatgactgaca |
| | | tagccaagaaaccaactgaaacgatttgtgcaccactcactgtcttttttgatggtagagttgatggtcaagtagacttatttagaaatgcccgtaatggtgttcttattacagaa |
| | | ggtagtgttaaaggtttacaaccatctgtaggtcccaaacaagctagtcttaatggagtcacattaattggagaagccgtaaaaacacagttcaattattataagaaagttgat |
| | | ggtgttgtccaacaattacctgaaacttactttactcagagtagaaatttacaagaatttaaacccaggagtcaaatggaaattgatttcttagaattagctatggatgaattcatt |
| | | gaacggtataaattagaaggctatgccttcgaacatatcgtttatggagattttagtcatagtcagttaggtggtttacatctactgattggactagctaaacgttttaaggaatca |
| | | catttgaattagaagattttattcctatggacagtacagttaaaaactatttcataacagatgcgcaaacaggttcatctaagtgtgtgtgttctgttattgatttattacttgatgatt |
| | | ttgttgaaataataaaatcccaagatttatctgtagtttctaaggttgtcaaagtgactattgactatacagaaatttcatttatgattggtgtaaagatggccatgtagaaacatttt |
| | | acccaaaattacaatctagtcaagcgtggcaaccgggtgttgctatgcctaatattacaaaatgcaaagaatgctattagaaaagtgtgaccttcaaaattatggtgatagtg |
| | | caacattacctaaaggcataatgatgaatgtcgcaaaatatactcaactgtgtcaatatttaaacacattaacattagctgtaccctataatatgagagttatacattttggtgctg |
| | | gttctgataaaggagttgcaccaggtacagctgttttaagacagtggttgcctacgggtacgctgcttgtcgattcagatcttaatgactttgtctctgatgcagattcaactttg |
| | | attggtgattgtgcaactgtacatacagctaataaatgggatctcattattagtgatatgtacgaccctaagactaaaaatgttacaaaagaaaatgactctaaagagggtttttt |
| | | cacttacatttgtgggtttatacaacaaaagctagctcttggaggttccgtggctataaagataacagaacattcttggaatgctgatctttataagctcatgggacacttcgcat |
| | | ggtggacagcctttgttactaatgtgaatgcgtcatcatctgaagcatttttaattggatgtaattatcttggcaaaccacgcgaacaaatagatggttatgtcatgcatgcaaat |
| | | tacatattttggaggaatacaaatccaattcagttgtcttcctattctttatttgacatgagtaaatttccccttaaattaaggggtactgctgttatgtattaaaagaaggtcaaatc |
| | | aatgatatgattttatctcttcttagtaaaggtagacttataattagagaaaacaacagagttgttatttctagtgatgttcttgttaacaactaaacgaacaatgtttgtttttcttgttt |
| | | tattgccactagtctctagtcagtgtgttaatcttacaaccagaactcaattaccccctgcatacactaattattcacacgtggtgtttattaccctgacaaagttttcagatcctca |
| | | gttttacattcaactcaggacttgttcttacctttatttccaatgttacttggttccatgctatacatgtctctgggaccaatggtactaagaggtttgataaccctgtcctaccattta |
| | | atgatggtgtttattttgcttccactgagaagtctaacataataagaggctggatttttggtactactttagattcgaagacccagtccctacttattgttaataacgctactaatgtt |
| | | gttattaaagtctgtgaatttcaattttgtaatgatccatttttgggtgtttattaccacaaaaacaacaaaagttggatggaaagtgagttcagagtttattctagtgcgaataattg |
| | | cacttttgaatatgtactcagccttttcttatggaccttgaaggaaaacagggtaatttcaaaaatcttagggaatttgtgtttaagaatattgatggttattttaaaatatattctaa |
| | | gcacacgcctattaatttagtgcgtgatctccctcagggtttttcggctttagaaccattggtagatttgccaataggtattaacatcactaggtttcaaactttacttgctttacata |
| | | gaagttatttgactcctggtgattcttcttcaggttggacagaggtgctgcagcttattatgtgggttatcttcaacctaggacttttctattaaaatataatgaaaatggaaccatt |
| | | acagatgctgtagactgtgcacttgaccctctctcagaaacaaagtgtacgttgaaatccttcactgtagaaaaaggaatctatcaaacttctaactttagagtccaaccaaca |
| | | gaatctattgttagatttcctaatattacaaacttgtgccatttggtgaagtttttaacgccaccagatttgcatctgtttatgcttggaacaggaagagaatcagcaactgtgttg |
| | | ctgattattctgtcctatataattccgcatcattttccacttttaagtgttatggagtgtctcctactaaattaaatgatactgattactaatgtctatgcagattcatttgtaattagag |
| | | gtgatgaagtcagacaaatcgctccagggcaaactggaaagattgctgattataattataaattaccagatgattttacaggctgcgttatagcttggaattctaacaatcttgat |
| | | tctaaggttggtggtaattataattacctgtatagattgtttaggaagtctaatctcaaaccttttgagagagatatttcaactgaaatctatcaggccggtagcacaccttgtaat |
| | | ggtgttgaaggttttaattgttactttcctttacaatcatatggtttccaacccactaatggtgttggttaccaaccatacagagtagtagtactttatttgaacttctacatgcacca |
| | | gcaactgtttgtggacctaaaaagtctactaatttggttaaaaacaaatgtgtcaatttcaacttcaatggtttaacaggcacaggtgttcttactgagtctaacaaaaagtttctg |
| | | cattccaacaatttggcagagacattgctgacactactgatgctgtccgtgatccacagacacttgagattcttgacattacaccatgttatttggtggtgtcagtgttataaca |
| | | ccaggaacaaatacttctaaccaggttgctgttattatcaggatgttaactgcacagaagtccctgttgctattcatgcagatcaacttactcctacttggcgtgtttattctaca |
| | | ggttctaatgtttttcaaacacgtgcaggctgtttaataggggctgaacatgtcaacaactcatatgagtgtgacatacccattggtgcaggtatatgcgctagttatcagactc |
| | | agactaattctcctcggcgggcacgtagtgtagctagtcaatccatcattgcctacactatgtcacttggtgcagaaaattcagttgcttactctaataactctattgccataccc |
| | | acaaattttactattagtgttaccacagaaattctaccagtgtctatgaccaagacatcagtagattgtacaatgtacatttgtggtgattcaactgaatgcagcaatcttttgttgc |
| | | aatatggcagtttttgtacacaattaaaccgtgattaactggaatagctgttgaacaagacaaaaacacccaagaagtttttgcacaagtcaaacaaatttacaaaacaccac |
| | | caattaaagattttggtggttttaatttttcacaaatattaccagatccatcaaaaccaagcaagaggtcatttattgaagatctacttttcaacaaagtgacacttgcagatgctg |
| | | gcttcatcaaacaatatggtgattgccttggtgatattgctgctagagacctcatttgtgcacaaaagtttaacggccttactgttttgccacctttgctcacagatgaaatgattg |
| | | ctcaatacacttctgcactgttagcgggtacaatcacttctggttggacctttggtgcaggtgctgcattacaaataccatttgctatgcaaatggcttataggtttaatggtattg |
| | | gagttacacagaatgttctctatgagaaccaaaaattgattgccaaccaatttaatagtgctattggcaaaattcaagactcactttcttccacagcaagtgcacttggaaaactt |
| | | caagatgtggtcaaccaaaatgcacaagctttaaacacgcttgttaaacaacttagctccaattttggtgcaatttcaagtgttttaaatgatatcctttcacgtcttgacaaagtt |
| | | gaggctgaagtgcaaattgataggttgatcacaggcagacttcaaagtttgcagacatatgtgactcaacaattaattagagctgcagaaatcagagcttctgctaatcttgct |
| | | gctactaaaatgtcagagtgtgtacttggacaatcaaaaagagttgatttttgtggaaagggctatcatcttatgtccttccctcagtcagcacctcatggtgtagtcttcttgcat |
| | | gtgacttatgtccctgcacaagaaaagaacttcacaactgctcctgccatttgtcatgatggaaaagcacactttcctcgtgaaggtgtctttgtttcaaatggcacacactggt |
| | | ttgtaacacaaaggaatttttatgaaccacaaatcattactacagacaacacatttgtgtaggtaactgtgatgttgtaataggaattgtcaacaacacagtttatgatcct-ttgc |
| | | aacctgaattagactcattcaaggaggagttagataaatattttaagaatcatacatcaccagatgttgatttaggtgacatctctggcattaatgcttcagttgtaaacattcaaa |
| | | aagaaattgaccgcctcaatgaggttgccaagaatttaaatgaatctctcatcgatctccaagaacttggaaagtatgagcagtatataaaatggccatggtacatttggctag |
| | | gttttatagaggcttgattgccatagtaatggtgacaattatgctttgctgtatgaccagttgctgtagttgtctcaagggctgttgttcttgtggatcctgctgcaaatttgatgaa |
| | | gacgactctgagccagtgctcaaaggagtcaaattacattacacataaacgaacttatggatttgtttatgagaatcttcacaattggaactgtaactttgaagcaaggtgaaat |
| | | caaggatgctactccttcagattttgttcgcgctactgcaacgataccgatacaagcctcactccctttcggatggcttattgttggcgttgcacttcttgctgtttttcagagcgc |
| | | ttccaaaatcataaccctcaaaaagagatggcaactagcactctccaagggtgttcactttgtttgcaacttgctgttgttgtttgtaacagtttactcacaccttttgctcgttgct |
| | | gctggccttgaagccccttttctctatctttatgctttagtctacttcttgcagagtataaactttgtaagaataataatgaggctttggctttgctggaaatgccgttccaaaaacc |
| | | cattactttatgatgccaactattttctttgctggcatactaattgttacgactattgtataccttacaatagtgtaacttcttcaattgtcattacttcaggtgatggcacaacaagtcc |
| | | tatttctgaacatgactaccagattggtggttatactgaaaaatgggaatctggagtaaaagactgtgttgtattacacagttacttcacttcagactattaccagctgtactcaac |
| | | tcaattgagtacagacactggtgttgaacatgttaccttcttcatctacaataaaattgttgatgagcctgaagaacatgtccaaattcacacaatcgacggttcatccggagtt |
| | | gttaatccagtaatggaaccaatttatgatgaaccgacgacgactactagcgtgcctttgtaagcacaagctgatgagtacgaacttatgtactcattcgtttcggaagagaca |
| | | ggtacgttaatagttaatagcgtacttattttcttgattcgtggtattcttgctagttacactagccatccttactgcgcttcgattgtgtgcgtactgctgcaatattgttaacgtga |
| | | gtcttgtaaaaccttattttacgtttactdcgtgttaaaaatctgaattcttctagagttcctgatcttctggtctaaacgaactaaatattatattagtttttctgtttggaactttaattt |
| | | tagccatggcagattccaacggtactattaccgttgaagagcttaaaaagctccttgaacaatggaacctagtaataggtttcctattccttacatggatttgtcttctacaatttg |
| | | cctatgccaacaggaataggtttttgtatataattaagttaattttcctaggctgttatggccagtaactttagcttgttttgtgcttgctgctgtttacagaataaattggatcaccg |
| | | gtggaattgctatcgcaatggcttgtcttgtaggcttgatgtggctcagctacttcattgcttctttcagactgtttgcgcgtacgcgttccatgtggtcattcaatccagaaacta |
| | | acattcttctcaacgtgccactccatggcactattctgaccagaccgcttctagaaagtgaactcgtaatcggagctgtgatccttcgtggacatcttcgtattgctggacacca |
| | | tctaggacgctgtgacatcaaggacctgcctaaagaaatcactgttgctacatcacgaacgctttcttattacaaattgggagcttcgcagcgtgtagcaggtgactcaggttt |
| | | tgctgcatacagtcgctacaggattggcaactataaattaaacacagaccattccagtagcagtgacaatattgattgcttgtacagtaagtgacaacagatgtttcatctcgt |
| | | tgactttcaggttactatagcagagatattactaattattatgaggacttttaaagtttccatttggaatcttgattacatcataaacctcataattaaaaatttatctaagtcactaact |
| | | gagaataaatattctcaattagatgaagagcaaccaatggagattgattaaacgaacatgaaaattattcttttcttggcactgataacactcgctacttgtgagattatcactac |
| | | caagagtgtgttagaggtacaacagtacttttaaaagaaccttgacttctggaacatacgagggcaattcaccatttcatcctctagctgataacaaatttgcactgacttgctt |
| | | tagcactcaatttgatttgcttgtcctgacggcgtaaaacacgtctatcagttacgtgccagatcagtttcacctaaactgttcatcagacaagaggaagttcaagaactttact |
| | | ctccaatttttcttattgttgcggcaatagtgtttataacactttgatcacactcaaaagaaagacagaatgattgaactttcattaattgacttctatttgtgattttagcctttctgc |
| | | tattccttgttttaattatgcttattatcttttggttctcacttgaactgcaagatcataatgaaacttgtcacgcctaaacgaacatgaaatttcttgttttcttaggaatcatcacaact |
| | | gtagctgcatttcaccaagaatgtagtttacagtcatgtactcaacatcaaccatatgtagttgatgacccgtgtcctattcacttctattctaaatggtatattagagtaggagct |
| | | agaaaatcagcacctttaattgaattgtgcgtggatgaggctggttctaaatcacccattcagtacatcgatatcggtaattatacagtttcctgtttaccttttacaattaattgcc |
| | | aggaacctaaattgggtagtcttgtagtgcgttgttcgttctatgaagactttttagagtatcatgacgttcgtgttgttttagatttcatctaaacgaacaaactaaaatgtctgata |
| | | atggaccccaaaatcagcgaaatgcaccccgcattacgtttggtggaccdcagattcaactggcagtaaccagaatggagaacgcagtggggcgcgatcaaaacaac |
| | | gtcggccccaaggtttacccaataatactgcgtcttggttcaccgctctcactcaacatggcaaggaagaccttaaattccctcgaggacaaggcgttccaattaacaccaat |
| | | agcagtccagatgaccaaattggctactaccgaagagctaccagacgaattcgtggtggtgacggtaaaatgaaagatctcagtccaagatggtatttctactacctagga |
| | | actgggccagaagctggacttccctatggtgctaacaaagacggcatcatatgggttgcaactgagggagccttgaatacaccaaaagatcacattggcacccgcaatcc |
| | | tgctaacaatgctgcaatcgtgctacaacttcctcaaggaacaacattgccaaaaggcttctacgcagaagggagcagaggcggcagtcaagcctcttctcgttcctcatc |
| | | acgtagtcgcaacagttcaagaaattcaactccaggcagcagtaggggaacttctcctgctagaatggctggcaatggcggtgatgctgctcttgattgctgctgcttgac |
| | | agattgaaccagcttgagagcaaaatgtaggtaaaggccaacaacaacaaggccaaactgtcactaagaaatctgctgctgaggcttctaagaagcctcggcaaaaac |
| | | gtactgccactaaagcatacaatgtaacacaagattcggcagacgtggtccagaacaaacccaaggaaattttggggaccaggaactaatcagacaaggaactgattac |
| | | aaacattggccgcaaattgcacaatttgcccccagcgcttcagcgttcttcggaatgtcgcgcattggcatggaagtcacaccttcgggaacgtggttgacctacacaggtg |
| | | ccatcaaattggatgacaaagatccaaatttcaaagatcaagtcattttgctgaataagcatattgacgcatacaaaacattcccaccaacagagcctaaaaaggacaaaaa |
| | | gaagaaggctgatgaaactcaagccttaccgcagagacagaagaaacagcaaactgtgactatcttcctgctgcagatttggatgatttctccaaacaattgcaacaatcc |
| | | atgagcagtgctgactcaactcaggcctaaactcatgcagaccacacaaggcagatgggctatataaacgttttcgcttttccgtttacgatatatagtctactcttgtgcagaa |
| | | tgaattctcgtaactacatagcacaagtagatgtagttaactttaatctcacatagcaatctttaatcagtgtgtaacattagggaggacttgaaagagccaccacattttcaccg |
| | | aggccacgcggagtacgatcgagtgtacagtgaacaatgctagggagagctgcctatatggaagagccctaatgtgtaaaattaattttagtagtgctatccccatgtgattt |
| | | taatagcttcttaggagaatgacaaaaaaaaaaaaaaaaaaaaaaaaaaaaaaaaa |
| 2408 | SARS-CoV- | atattaggtttttacctacccaggaaaagccaaccaacctcgatctcttgtagatctgttctctaaacgaactttaaaatctgtgtagctgtcgctcggctgcatgcctagtgcac |
| | 1 genome | ctacgcagtataaacaataataaattttactgtcgttgacaagaaacgagtaactcgtccctcttctgcagactgcttacggtttcgtccgtgttgcagtcgatcatcagcatacc |
| | (Genbank | taggtttcgtccgggtgtgaccgaaaggtaagatggagagccttgttcttggtgtcaacgagaaaacacacgtccaactcagtttgcctgtccttcaggttagagacgtgcta |
| | Accession | gtgcgtggcttcggggactctgtggaagaggccctatcggaggcacgtgaacacctcaaaaatggcacttgtggtctagtagagctggaaaaaggcgtactgccccagc |
| | No. | ttgaacagccctatgtgttcattaaacgttctgatgccttaagcaccaatcacggccacaaggtcgttgagctggttgcagaaatggacggcattcagtacggtcgtagcggt |
| | NC_ | ataacactgggagtactcgtgccacatgtgggcgaaaccccaattgcataccgcaatgttcttcttcgtaagaacggtaataagggagccggtggtcatagctatggcatc |
| | 004718.3) | gatctaaagtcttatgacttaggtgacgagcttggcactgatcccattgaagattatgaacaaaactggaacactaagcatggcagtggtgcactccgtgaactcactcgtga |
| | | gctcaatggaggtgcagtcactcgctatgtcgacaacaatttctgtggcccagatgggtaccctcttgattgcatcaaagattttctcgcacgcgcgggcaagtcaatgtgca |
| | | ctctttccgaacaacttgattacatcgagtcgaagagaggtgtctactgctgccgtgaccatgagcatgaaattgcctggttcactgagcgctctgataagagctacgagca |
| | | ccagacacccttcgaaattaagagtgccaagaaatttgacactttcaaaggggaatgcccaaagtttgtgtttcctcttaactcaaaagtcaaagtcattcaaccacgtgttga |
| | | aaagaaaaagactgagggtttcatggggcgtatacgctctgtgtaccctgttgcatctccacaggagtgtaacaatatgcacttgtctaccttgatgaaatgtaatcattgcga |
| | | tgaagtttcatggcagacgtgcgactttctgaaagccacttgtgaacattgtggcactgaaaatttagttattgaaggacctactacatgtgggtacctacctactaatgctgta |
| | | gtgaaaatgccatgtcctgcctgtcaagacccagagattggacctgagcatagtgttgcagattatcacaaccactcaaacattgaaactcgactccgcaagggaggtagg |
| | | actagatgttttggaggctgtgtgtttgcctatgttggctgctataataagcgtgcctactgggttcctcgtgctagtgctgatattggctcaggccatactggcattactggtga |
| | | caatgtggagaccttgaatgaggatctccttgagatactgagtcgtgaacgtgttaacattaacattgttggcgattttcatttgaatgaagaggttgccatcattttggcatcttt |
| | | ctctgcttctacaagtgcctttattgacactataaagagtcttgattacaagtattcaaaaccattgttgagtcctgcggtaactataaagttaccaagggaaagcccgtaaaag |
| | | gtgcttggaacattggacaacagagatcagttttaacaccactgtgtggttttccctcacaggctgctggtgttatcagatcaatttttgcgcgcacacttgatgcagcaaacca |
| | | ctcaattcctgatttgcaaagagcagctgtcaccatacttgatggtatttctgaacagtcattacgtcttgtcgacgccatggtttatacttcagacctgctcaccaacagtgtcat |
| | | tattatggcatatgtaactggtggtcttgtacaacagacttctcagtggttgtctaatcttttgggcactactgttgaaaaactcaggcctatctttgaatggattgaggcgaaact |
| | | tagtgcaggagttgaatttctcaaggatgcttgggagattctcaaatttctcattacaggtgtttttgacatcgtcaagggtcaaatacaggttgcttcagataacatcaaggatt |
| | | gtgtaaaatgcttcattgatgttgttaacaaggcactcgaaatgtgcattgatcaagtcactatcgctggcgcaaagttgcgatcactcaacttaggtgaagtcttcatcgctca |
| | | aagcaagggactttaccgtcagtgtatacgtggcaaggagcagctgcaactactcatgcctcttaaggcaccaaaagaagtaacctttcttgaaggtgattcacatgacaca |
| | | gtacttacctctgaggaggttgttctcaagaacggtgaactcgaagcactcgagacgcccgttgatagcttcacaaatggagctatcgttggcacaccagtagtgtaaatg |
| | | gcctcatgacttagagattaaggacaaagaacaatactgcgcattgtctcctggtttactggctacaaacaatgtattcgcttaaaagggggtgcaccaattaaaggtgtaa |
| | | cattggagaagatactgtttgggaagttcaaggttacaagaatgtgagaatcacatttgagcttgatgaacgtgttgacaaagtgcttaatgaaaagtgactgtctacactgt |
| | | tgaatccggtaccgaagttactgagtttgcatgtgttgtagcagaggctgttgtgaagactttacaaccagtttctgatctccttaccaacatgggtattgatcttgatgagtgga |
| | | gtgtagctacattctacttatttgatgatgctggtgaagaaaacttttcatcacgtatgtattgttccttttaccctccagatgaggaagaagaggacgatgcagagtgtgagga |
| | | agaagaaattgatgaaacctgtgaacatgagtacggtacagaggatgattatcaaggtaccactggaatttggtgcctcagctgaaacagttcgagttgaggaagaaga |
| | | agaggaagactggctggatgatactactgagcaatcagagattgagccagaaccagaacctacacctgaagaaccagttaatcagtttactggttatttaaaacttactgac |
| | | aatgttgccattaaatgtgttgacatcgttaaggaggcacaaagtgctaatcctatggtgattgtaaatgctgctaacatacacctgaaacatggtggtggtgtagcaggtgca |
| | | ctcaacaaggcaaccaatggtgccatgcaaaaggagagtgatgattacattaagctaaatggccctcttacagtaggagggtcttgtttgattctggacataatcttgctaag |
| | | aagtgtctgcatgttgttggacctaacctaaatgcaggtgaggacatccagcttcttaaggcagcatatgaaaatttcaattcacaggacatcttacttgcaccattgttgtcag |
| | | caggcatatttggtgctaaaccacttcagtattacaagtgtgcgtgcagacggttcgtacacaggtttatattgcagtcaatgacaaagctctttatgagcaggttgtcatgga |
| | | ttatcttgataacctgaagcctagagtggaagcacctaaacaagaggagccaccaaacacagaagattccaaaactgaggagaaatctgtcgtacagaagcctgtcgatg |
| | | tgaagccaaaaattaaggcctgcattgatgaggttaccacaacactggaagaaactaagtttcttaccaataagttactcttgtttgctgatatcaatggtaagattaccatgat |
| | | tctcagaacatgcttagaggtgaagatatgtctttccttgagaaggatgcaccttacatggtaggtgatgttatcactagtggtgatatcacttgtgttgtaataccctccaaaaa |
| | | ggctggtggcactactgagatgctctcaagagctttgaagaaagtgccagttgatgagtatataaccacgtaccctggacaaggatgtgctggttatacacttgaggaagct |
| | | aagactgctcttaagaaatgcaaatctgcattttatgtactaccttcagaagcacctaatgctaaggaagagattctaggaactgtatcctggaatttgagagaaatgcttgctc |
| | | atgctgaagagacaagaaaattaatgcctatatgcatggatgttagagccataatggcaaccatccaacgtaagtataaaggaattaaaattcaagagggcatcgttgactat |
| | | ggtgtccgattcttcttttatactagtaaagagcctgtagcttctattattacgaagctgaactctctaaatgagccgcttgtcacaatgccaattggttatgtgacacatggtttta |
| | | atcttgaagaggctgcgcgctgtatgcgttctcttaaagctcctgccgtagtgtcagtatcatcaccagatgctgttactacatataatggatacctcacttcgtcatcaaagac |
| | | atctgaggagcactttgtagaaacagtttattggctggctcttacagagattggtcctattcaggacagcgtacagagttaggtgttgaatttcttaagcgtggtgacaaaatt |
| | | gtgtaccacactctggagagccccgtcgagtttcatcttgacggtgaggttctttcacttgacaaactaaagagtctcttatccctgcgggaggttaagactataaaagtgttc |
| | | acaactgtggacaacactaatctccacacacagcttgtggatatgtctatgacatatggacagcagtttggtccaacatacttggatggtgctgatgttacaaaaattaaacct |
| | | catgtaaatcatgagggtaagactttattgtactacctagtgatgacacactacgtagtgaagattcgagtactaccatactcttgatgagagttttcttggtaggtacatgtct |
| | | gattaaaccacacaaagaaatggaaatttcctcaagttggtggtttaacttcaattaaatgggctgataacaattgttatttgtctagtgttttattagcacttcaacagcttgaagt |
| | | caaattcaatgcaccagcacttcaagaggcttattatagagcccgtgctggtgatgctgctaacttttgtgcactcatactcgcttacagtaataaaactgttggcgagcttggt |
| | | gatgtcagagaaactatgacccatcttctacagcatgctaatttggaatctgcaaagcgagttcttaatgtggtgtgtaaacattgtggtcagaaaactactaccttaacgggtg |
| | | tagaagctgtgatgtatatgggtactctatcttatgataatcttaagacaggtgtttccattccatgtgtgtgtggtcgtgatgctacacaatatctagtacaacaagagtcttctttt |
| | | gttatgatgtctgcaccacctgctgagtataaattacagcaaggtacattcttatgtgcgaatgagtacactggtaactatcagtgtggtcattacactcatataactgctaagga |
| | | gaccactatcgtattgacggagctcaccttacaaagatgtcagagtacaaaggaccagtgactgatgttttctacaaggaaacatcttacactacaaccatcaagcctgtgt |
| | | cgtataaactcgatggagttacttacacagagattgaaccaaaattggatgggtattataaaaaggataatgcttactatacagagcagcctatagaccttgtaccaactcaac |
| | | cattaccaaatgcgagttttgataatttcaaactcacatgttctaacacaaaatttgctgatgatttaaatcaaatgacaggcttcacaaagccagcttcacgagagctatctgtc |
| | | acattcttcccagacttgaatggcgatgtagtggctattgactatagacactattcagcgagtttcaagaaaggtgctaaattactgcataagccaattgtttggcacattaacc |
| | | aggctacaaccaagacaacgttcaaaccaaacacttggtgtttacgttgtctttggagtacaaagccagtagatacttcaaattcatttgaagttctggcagtagaagacacac |
| | | aaggaatggacaatcttgcttgtgaaagtcaacaacccacctctgaagaagtagtggaaaatcctaccatacagaaggaagtcatagagtgtgacgtgaaaactaccgaa |
| | | gttgtaggcaatgtcatacttaaaccatcagatgaaggtgttaaagtaacacaagagttaggtcatgaggatcttatggctgcttatgtggaaaacacaagcattaccattaag |
| | | aaacctaatgagctttcactagccttaggtttaaaaacaattgccactcatggtattgctgcaattaatagtgttccttggagtaaaattttggcttatgtcaaaccattcttaggac |
| | | aagcagcaattacaacatcaaattgcgctaagagattagcacaacgtgtgtttaacaattatatgccttatgtgtttacattattgttccaattgtgtacttttactaaaagtaccaat |
| | | tctagaattagagcttcactacctacaactattgctaaaaatagtgttaagagtgttgctaaattatgtttggatgccggcattaattatgtgaagtcacccaaattttctaaattgtt |
| | | cacaatcgctatgtggctattgttgttaagtatttgcttaggttactaatctgtgtaactgctgatttggtgtactcttatctaattttggtgctccttcttattgtaatggcgttagag |
| | | aattgtatcttaattcgtctaacgttactactatggatttctgtgaaggttatttccttgcagcatttgtttaagtggattagactcccttgattcttatccagctcttgaaaccattcag |
| | | gtgacgatttcatcgtacaagctagacttgacaattttaggtctggccgctgagtgggttttggcatatatgttgttcacaaaattatttatttattaggtctttcagctataatgca |
| | | ggtgttctttggctattttgctagtcatttcatcagcaattcttggctcatgtggtttatcattagtattgtacaaatggcacccgtttctgcaatggttaggatgtacatcttctttgctt |
| | | ctttctactacatatggaagagctatgttcatatcatggatggttgcacctcttcgacttgcatgatgtgctataagcgcaatcgtgccacacgcgttgagtgtacaactattgtt |
| | | aatggcatgaagagatctttctatgtctatgcaaatggaggccgtggcttctgcaagactcacaattggaattgtctcaattgtgacacattttgcactggtagtacattcattag |
| | | tgatgaagttgctcgtgatttgtcactccagtttaaaagaccaatcaaccctactgaccagtcatcgtatattgttgatagtgttgctgtgaaaaatggcgcgcttcacctctactt |
| | | tgacaaggctggtcaaaagacctatgagagacatccgctctcccattttgtcaatttagacaatttgagagctaacaacactaaaggttcactgcctattaatgtcatagtttttg |
| | | atggcaagtccaaatgcgacgagtctgcttctaagtctgcttctgtgtactacagtcagctgatgtgccaacctattctgttgcttgaccaagctcttgtatcagacgttggaga |
| | | tagtactgaagtttccgttaagatgtttgatgcttatgtcgacaccttttcagcaacttttagtgttcctatggaaaaacttaaggcacttgttgctacagctcacagcgagttagc |
| | | aaagggtgtagctttagatggtgtcctttctacattcgtgtcagctgcccgacaaggtgttgttgataccgatgttgacacaaaggatgttattgaatgtctcaaactttcacatc |
| | | actctgacttagaagtgacaggtgacagttgtaacaatttcatgctcacctataataaggttgaaaacatgacgcccagagatcttggcgcatgtattgactgtaatgcaagg |
| | | catatcaatgcccaagtagcaaaaagtcacaatgtttcactcatctggaatgtaaaagactacatgtctttatctgaacagctgcgtaaacaaattcgtagtgctgccaagaag |
| | | aacaacataccttttagactaacttgtgctacaactagacaggttgtcaatgtcataactactaaaatctcactcaagggtggtaagattgttagtacttgttttaaacttatgctta |
| | | aggccacattattgtgcgttcttgctgcattggtttgttatatcgttatgccagtacatacattgtcaatccatgatggttacacaaatgaaatcattggttacaaagccattcagga |
| | | tggtgtcactcgtgacatcatttctactgatgattgttttgcaaataaacatgctggttttgacgcatggtttagccagcgtggtggttcatacaaaaatgacaaaagctgccctg |
| | | tagtagctgctatcattacaagagagattggtttcatagtgcctggcttaccgggtactgtgctgagagcaatcaatggtgacttcttgcattttctacctcgtgtttttagtgctgt |
| | | tggcaacatttgctacacaccttccaaactcattgagtatagtgattttgctacctctgcttgcgttcttgctgctgagtgtacaatttttaaggatgctatgggcaaacctgtgcc |
| | | atattgttatgacactaatttgctagagggttctatttcttatagtgagcttcgtccagacactcgttatgtgcttatggatggttccatcatacagtttcctaacacttacctggagg |
| | | gttctgttagagtagtaacaacttttgatgctgagtactgtagacatggtacatgcgaaaggtcagaagtaggtatttgcctatctaccagtggtagatgggttcttaataatga |
| | | gcattacagagctctatcaggagttttctgtggtgttgatgcgatgaatctcatagctaacatctttactcctcttgtgcaacctgtgggtgattagatgtgtctgcttcagtagtg |
| | | gctggtggtattattgccatattggtgacttgtgctgcctactactttatgaaattcagacgtgtttttggtgagtacaaccatgttgttgctgctaatgcacttttgtttttgatgtcttt |
| | | cactatactagtaggtaccagcttacagctttctgccgggagtctactcagtatttacttgtacttgacattctatttcaccaatgatgtttcattcttggctcaccttcaatggttt |
| | | gccatgttttctcctattgtgcctttttggataacagcaatctatgtattctgtatttctctgaagcactgccattggttattaacaactatcttaggaaaagagtcatgtttaatgga |
| | | gttacatttagtaccttcgaggaggctgattgtgtacctttttgctcaacaaggaaatgtacctaaaattgcgtagcgagacactgttgccacttacacagtataacaggtatct |
| | | tgactatataacaagtacaagtatttcagtggagccttagatactaccagctatcgtgaagcagcttgctgccacttagcaaaggctctaaatgactttagcaactcaggtgct |
| | | gatgttactaccaaccaccacagacatcaatcacttctgctgttctgcagagtggttttaggaaaatggcattcccgtcaggcaaagttgaagggtgcatggtacaagtaac |
| | | ctgtggaactacaactcttaatggattgtggttggatgacacagtatactgtccaagacatgtcatttgcacagcagaagacatgcttaatcctaactatgaagatctgctcatt |
| | | cgcaaatccaaccatagctttcttgttcaggctggcaatgttcaacttcgtgttattggccattctatgcaaaattgtctgcttaggcttaaagttgatacttctaaccctaagacac |
| | | ccaagtataaatttgtccgtatccaacctggtcaaacattttcagttctagcatgctacaatggttcaccatctggtgtttatcagtgtgccatgagacctaatcataccattaaag |
| | | gttattccttaatggatcatgtggtagtgttggttttaacattgattatgattgcgtgtctttctgctatatgcatcatatggagcttccaacaggagtacacgctggtactgactta |
| | | gaaggtaaattctatggtccatttgttgacagacaaactgcacaggctgcaggtacagacacaaccataacattaaatgttttggcatggctgtatgctgctgttatcaatggt |
| | | gataggtggtttcttaatagattcaccactactttgaatgactttaaccttgtggcaatgaagtacaactatgaacctttgacacaagatcatgttgacatattgggacctctttctg |
| | | ctcaaacaggaattgccgtcttagatatgtgtgctgattgaaagagctgctgcagaatggtatgaatggtcgtactatccttggtagcactattttagaagatgagtttacacc |
| | | atttgatgttgttagacaatgactggtgttaccttccaaggtaagttcaagaaaattgttaagggcactcatcattggatgatttaactttcttgacatcactattgattcttgttca |
| | | aagtacacagtggtcactgtttttctttgtttacgagaatgattcttgccatttactcttggtattatggcaattgctgcatgtgctatgctgcttgttaagcataagcacgcattctt |
| | | gtgcttgtttctgttaccttctcttgcaacagttgcttactttaatatggtctacatgcctgctagctgggtgatgcgtatcatgacatggcttgaattggctgacactagcttgtctg |
| | | gttataggcttaaggattgtgttatgtatgcttcagctttagttttgcttattctcatgacagctcgcactgtttatgatgatgctgctagacgtgtttggacactgatgaatgtcatta |
| | | cacttgtttacaaagtctactatggtaatgattagatcaagctatttccatgtgggccttagttatttctgtaacctctaactattctggtgtcgttacgactatcatgtttttagctag |
| | | agctatagtgtttgtgtgtgttgagtattacccattgttatttattactggcaacaccttacagtgtatcatgcttgtttattgtttcttaggctattgttgctgctgctactttggccttttc |
| | | tgtttactcaaccgttacttcaggcttactcttggtgtttatgactacttggtactacacaagaatttaggtatatgaactcccaggggcttttgcctcctaagagtagtattgatg |
| | | ctttcaagcttaacattaagttgttgggtattggaggtaaaccatgtatcaaggttgctactgtacagtctaaaatgtctgacgtaaagtgcacatctgtggtactgctctcggttc |
| | | ttcaacaacttagagtagagtcatcttctaaattgtgggcacaatgtgtacaactccacaatgatattcttcttgcaaaagacacaactgaagctttcgagaagatggtttctcttt |
| | | tgtctgttttgctatccatgcagggtgctgtagacattaataggttgtgcgaggaaatgctcgataaccgtgctactcttcaggctattgcttcagaatttagttattaccatcata |
| | | tgccgcttatgccactgcccaggaggcctatgagcaggctgtagctaatggtgattctgaagtcgttctcaaaaagttaaagaaatctttgaatgtggctaaatctgagtttga |
| | | ccgtgatgctgccatgcaacgcaagttggaaaagatggcagatcaggctatgacccaaatgtacaaacaggcaagatctgaggacaagagggcaaaagtaactagtgc |
| | | tatgcaaacaatgctcttcactatgcttaggaagcttgataatgatgcacttaacaacattatcaacaatgcgcgtgatggttgtgttccactcaacatcataccattgactacag |
| | | cagccaaactcatggttgttgtccctgattatggtacctacaagaacacttgtgatggtaacacctttacatatgcatctgcactagggaaatccagcaagttgttgatgcgga |
| | | tagcaagattgttcaacttagtgaaattaacatggacaattcaccaaatttggcttggcctcttattgttacagctctaagagccaactcagctgttaaactacagaataatgaac |
| | | tgagtccagtagcactacgacagatgtcctgtgcggctggtaccacacaaacagcttgtactgatgacaatgcacttgcctactataacaattcgaagggaggtaggtttgt |
| | | gctggcattactatcagaccaccaagatctcaaatgggctagattccctaagagtgatggtacaggtacaatttacacagaactggaaccaccttgtaggtttgttacagaca |
| | | caccaaaagggcctaaagtgaaatacttgtacttcatcaaaggcttaaacaacctaaatagaggtatggtgctgggcagtttagctgctacagtacgtcttcaggctggaaat |
| | | gctacagaagtacctgccaattcaactgtgattccttctgtgatttgcagtagaccctgctaaagcatataaggattacctagcaagtggaggacaaccaatcaccaactgt |
| | | gtgaagatgttgtgtacacacactggtacaggacaggcaattactgtaacaccagaagctaacatggaccaagagtcctttggtggtgcttcatgttgtagtattgtagatgc |
| | | cacattgaccatccaaatcctaaaggattctgtgacttgaaaggtaagtacgtccaaatacctaccacttgtgctaatgacccagtgggttttacacttagaaacacagtagta |
| | | ccgtctgcggaatgtggaaaggttatggctgtagttgtgaccaactccgcgaacccttgatgcagtctgcggatgcatcaacgtttttaaacgggtttgcggtgtaagtgcag |
| | | cccgtcttacaccgtgcggcacaggcactagtactgatgtcgtctacagggcttttgatatttacaacgaaaaagttgctggttttgcaaagttcctaaaaactaattgctgtcg |
| | | cttccaggagaaggatgaggaaggcaatttattagactcttactttgtagttaagaggcatactatgtctaactaccaacatgaagagactatttataacttggttaaagattgtc |
| | | cagcggttgctgtccatgactttttcaagtttagagtagatggtgacatggtaccacatatatcacgtcagcgtctaactaaatacacaatggctgatttagtctatgctctacgt |
| | | cattttgatgagggtaattgtgatacattaaaagaaatactcgtcacatacaattgctgtgatgatgattatttcaataagaaggattggtatgacttcgtagagaatcctgacatc |
| | | ttacgcgtatatgctaacttaggtgagcgtgtacgccaatcattattaaagactgtacaattctgcgatgctatgcgtgatgcaggcattgtaggcgtactgacattagataatc |
| | | aggatcttaatgggaactggtacgatttcggtgatttcgtacaagtagcaccaggctgcggagttcctattgtggattcatattactcattgctgatgcccatcctcactttgact |
| | | agggcattggctgctgagtcccatatggatgctgatctcgcaaaaccacttattaagtgggatttgctgaaatatgattttacggaagagagactttgtctcttcgaccgttattt |
| | | taaatattgggaccagacataccatcccaattgtattaactgtttggatgataggtgtatccttcattgtgcaaactttaatgtgttattttctactgtgtttccacctacaagttttgg |
| | | accactagtaagaaaaatatttgtagatggtgttccttttgttgtttcaactggataccattttcgtgagttaggagtcgtacataatcaggatgtaaacttacatagctcgcgtctc |
| | | agtttcaaggaacttttagtgtatgctgctgatccagctatgcatgcagcttctggcaatttattgctagataaacgcactacatgatttcagtagctgcactaacaaacaatgtt |
| | | gcttttcaaactgtcaaacccggtaattttaataaagacttttatgactttgctgtgtctaaaggtttctttaaggaaggaagttctgttgaactaaaacacttcttctttgctcaggat |
| | | ggcaacgctgctatcagtgattatgactattatcgttataatctgccaacaatgtgtgatatcagacaactcctattcgtagttgaagttgttgataaatactttgattgttacgatg |
| | | gtggctgtattaatgccaaccaagtaatcgttaacaatctggataaatcagctggtttcccatttaataaatggggtaaggctagactttattatgactcaatgagttatgaggat |
| | | caagatgcacttttcgcgtatactaagcgtaatgtcatccctactataactcaaatgaatcttaagtatgccattagtgcaaagaatagagctcgcaccgtagaggtgtactat |
| | | ctgtagtactatgacaaatagacagtttcatcagaaattattgaagtcaatagccgccactagaggagctactgtggtaattggaacaagcaagttttacggtggctggcata |
| | | atatgttaaaaactgtttacagtgatgtagaaactccacaccttatgggttgggattatccaaaatgtgacagagccatgcctaacatgcttaggataatggcctctcttgttctt |
| | | gctcgcaaacataacacttgctgtaacttatcacaccgtttctacaggttagctaacgagtgtgcgcaagtattaagtgagatggtcatgtgtggcggctcactatatgttaaac |
| | | caggtggaacatcatccggtgatgctacaactgcttatgctaatagtgtctttaacatttgtcaagctgttacagccaatgtaaatgcacttattcaactgatggtaataagata |
| | | gctgacaagtatgtccgcaatctacaacacaggctctatgagtgtactatagaaatagggatgttgatcatgaattcgtggatgagttttacgcttacctgcgtaaacatttctc |
| | | catgatgattattctgatgatgccgttgtgtgctataacagtaactatgcggctcaaggtttagtagctagcattaagaactttaaggcagttattattatcaaaataatgtgttc |
| | | atgtctgaggcaaaatgttggactgagactgaccttactaaaggacctcacgaattttgctcacagcatacaatgctagttaaacaaggagatgattacgtgtacctgccttac |
| | | ccagatccatcaagaatattaggcgcaggctgttttgtcgatgatattgtcaaaacagatggtacacttatgattgaaaggttcgtgtcactggctattgatgcttacccacttac |
| | | aaaacatcctaatcaggagtatgctgatgtctttcacttgtatttacaatacattagaaagttacatgatgagcttactggccacatgttggacatgtattccgtaatgctaactaat |
| | | gataacacctcacggtactgggaacctgagttttatgaggctatgtacacaccacatacagtcttgcaggctgtaggtgcttgtgtattgtgcaattcacagacttcacttcgtt |
| | | gcggtgcctgtattaggagaccattcctatgttgcaagtgctgctatgaccatgtcatttcaacatcacacaaattagtgttgtctgttaatccctatgtttgcaatgccccaggtt |
| | | gtgatgtcactgatgtgacacaactgtatctaggaggtatgagctattattgcaagtcacataagcctcccattagttttccattatgtgctaatggtcaggtttttggtttatacaa |
| | | aaacacatgtgtaggcagtgacaatgtcactgacttcaatgcgatagcaacatgtgattggactaatgctggcgattacatacttgccaacacttgtactgagagactcaagc |
| | | ttttcgcagcagaaacgctcaaagccactgaggaaacatttaagctgtcatatggtattgccactgtacgcgaagtactctctgacagagaattgcatctttcatgggaggttg |
| | | gaaaacctagaccaccattgaacagaaactatgtattactggttaccgtgtaactaaaaatagtaaagtacagattggagagtacacctttgaaaaaggtgactatggtgat |
| | | gctgttgtgtacagaggtactacgacatacaagttgaatgttggtgattactttgtgttgacatctcacactgtaatgccacttagtgcacctactctagtgccacaagagcact |
| | | atgtgagaattactggcttgtacccaacactcaacatctcagatgagttttctagcaatgttgcaaattatcaaaaggtcggcatgcaaaagtactctacactccaaggaccac |
| | | ctggtactggtaagagtcattttgccatcggacttgactctattacccatctgctcgcatagtgtatacggcatgctctcatgcagctgttgatgccctatgtgaaaaggcatta |
| | | aaatatttgcccatagataaatgtagtagaatcatacctgcgcgtgcgcgcgtagagtgttttgataaattcaaagtgaattcaacactagaacagtatgttttagcactgtaaa |
| | | tgcattgccagaaacaactgctgacattgtagtctttgatgaaatctctatggctactaattatgacttgagtgttgtcaatgctagacttcgtgcaaaacactacgtctatattgg |
| | | cgatcctgctcaattaccagccccccgcacattgctgactaaaggcacactagaaccagaatattttaattcagtgtgcagacttatgaaaacaataggtccagacatgttcct |
| | | tggaacttgtcgccgttgtcctgctgaaattgttgacactgtgagtgattagtttatgacaataagctaaaagcacacaaggataagtcagctcaatgcttcaaaatgttctaca |
| | | aaggtgttattacacatgatgtttcatctgcaatcaacagacctcaaataggcgttgtaagagaatttcttacacgcaatcctgcttggagaaaagctgtttttatctcaccttata |
| | | attcacagaacgctgtagcttcaaaaatcttaggattgcctacgcagactgttgattcatcacagggttctgaatatgactatgtcatattcacacaaactactgaaacagcaca |
| | | ctcttgtaatgtcaaccgcttcaatgtggctatcacaagggcaaaaattggcattttgtgcataatgtctgatagagatctttatgacaaactgcaatttacaagtctagaaatac |
| | | cacgtcgcaatgtggctacattacaagcagaaaatgtaactggactttttaaggactgtagtaagatcattactggtcttcatcctacacaggcacctacacacctcagcgttg |
| | | atataaagttcaagactgaaggattatgtgttgacataccaggcataccaaaggacatgacctaccgtagactcatctctatgatgggtttcaaaatgaattaccaagtcaatg |
| | | gttaccctaatatgtttatcacccgcgaagaagctattcgtcacgttcgtgcgtggattggctttgatgtagagggctgtcatgcaactagagatgctgtgggtactaacctac |
| | | ctctccagctaggattttctacaggtgttaacttagtagctgtaccgactggttatgttgacactgaaaataacacagaattcaccagagttaatgcaaaacctccaccaggtg |
| | | accagtttaaacatcttataccactcatgtataaaggcttgccaggaatgtagtgcgtattaagatagtacaaatgctcagtgatacactgaaaggattgtcagacagagtcg |
| | | tgttcgtcctttgggcgcatggctttgagcttacatcaatgaagtactttgtcaagattggacctgaaagaacgtgttgtctgtgtgacaaacgtgcaacttgatttctacttcat |
| | | cagatacttatgcctgctggaatcattctgtgggttttgactatgtctataacccatttatgattgatgttcagcagtggggctttacgggtaaccttcagagtaaccatgaccaa |
| | | cattgccaggtacatggaaatgcacatgtggctagttgtgatgctatcatgactagatgtttagcagtccatgagtgctttgttaagcgcgttgattggtctgttgaataccctatt |
| | | ataggagatgaactgagggttaattctgcttgcagaaaagtacaacacatggttgtgaagtctgcattgcttgctgataagtttccagttcttcatgacattggaaatccaaagg |
| | | ctatcaagtgtgtgcctcaggctgaagtagaatggaagttctacgatgctcagccatgtagtgacaaagcttacaaaatagaggaactcttctattcttatgctacacatcacg |
| | | ataaattcactgatggtgtttgtttgttttggaattgtaacgttgatcgttacccagccaatgcaattgtgtgtaggtttgacacaagagtcttgtcaaacttgaacttaccaggctg |
| | | tgatggtggtagtttgtatgtgaataagcatgcattccacactccagctttcgataaaagtgcatttactaatttaaagcaattgcctttatttactattctgatagtccttgtgagtc |
| | | tcatggcaaacaagtagtgtcggatattgattatgttccactcaaatctgctacgtgtattacacgatgcaatttaggtggtgctgtttgcagacaccatgcaaatgagtaccga |
| | | cagtacttggatgcatataatatgatgatttctgctggatttagcctatggatttacaaacaatttgatacttataacctgtggaatacatttaccaggttacagagtttagaaaatg |
| | | tggcttataatgttgttaataaaggacactttgatggacacgccggcgaagcacctgtttccatcattaataatgctgtttacacaaaggtagatggtattgatgtggagatcttt |
| | | gaaaataagacaacacttcctgttaatgttgcatttgagctttgggctaagcgtaacattaaaccagtgccagagattaagatactcaataatttgggtgttgatatcgctgctaa |
| | | tactgtaatctgggactacaaaagagaagccccagcacatgtatctacaataggtgtctgcacaatgactgacattgccaagaaacctactgagagtgcttgttcttcacttac |
| | | tgtcttgtttgatggtagagtggaaggacaggtagacctttttagaaacgcccgtaatggtgttttaataacagaaggttcagtcaaaggtctaacaccttcaaagggaccag |
| | | cacaagctagcgtcaatggagtcacattaattggagaatcagtaaaaacacagtttaactactttaagaaagtagacggcattattcaacagttgcctgaaacctactttactc |
| | | agagcagagacttagaggattttaagcccagatcacaaatggaaactgactttctcgagctcgctatggatgaattcatacagcgatataagctcgagggctatgccttcga |
| | | acacatcgtttatggagatttcagtcatggacaacttggcggtcttcatttaatgataggcttagccaagcgctcacaagattcaccacttaaattagaggattttatccctatgg |
| | | acagcacagtgaaaaattacttcataacagatgcgcaaacaggttcatcaaaatgtgtgtgttctgtgattgatcttttacttgatgactttgtcgagataataaagtcacaagatt |
| | | tgtcagtgatttcaaaagtggtcaaggttacaattgactatgctgaaatttcattcatgattggtgtaaggatggacatgttgaaaccttctacccaaaactacaagcaagtcaa |
| | | gcgtggcaaccaggtgttgcgatgcctaacttgtacaagatgcaaagaatgcttcttgaaaagtgtgaccttcagaattatggtgaaaatgctgttataccaaaaggaataat |
| | | gatgaatgtcgcaaagtatactcaactgtgtcaatacttaaatacacttactttagctgtaccctacaacatgagagttattcactttggtgctggctctgataaaggagttgcac |
| | | caggtacagagtgctcagacaatggttgccaactggcacactacttgtcgattcagatcttaatgacttcgtctccgacgcagattctactttaattggagactgtgcaacagt |
| | | acatacggctaataaatgggaccttattattagcgatatgtatgaccctaggaccaaacatgtgacaaaagagaatgactctaaagaagggtttttcacttatctgtgtggattt |
| | | ataaagcaaaaactagccagggtggttctatagctgtaaagataacagagcattcttggaatgctgacctttacaagcttatgggccatttctcatggtggacagcttttgtta |
| | | caaatgtaaatgcatcatcatcggaagcatttttaattggggctaactatcttggcaagccgaaggaacaaattgatggctataccatgcatgctaactacattttctggagga |
| | | acacaaatcctatccagttgtcttcctattcactctttgacatgagcaaatttcctcttaaattaagaggaactgctgtaatgtctcttaaggagaatcaaatcaatgatatgatttat |
| | | tctcttctggaaaaaggtaggcttatcattagagaaaacaacagagttgtggtttcaagtgatattcttgttaacaactaaacgaacatgtttattttcttattatttcttactctcact |
| | | agtggtagtgaccttgaccggtgcaccacttttgatgatgttcaagctcctaattacactcaacatacttcatctatgaggggggtttactatcctgatgaaatttttagatcagac |
| | | actctttatttaactcaggatttatttcttccattttattctaatgttacagggtttcatactattaatcatacgtttggcaaccctgtcataccttttaaggatggtatttattttgctgcca |
| | | cagagaaatcaaatgttgtccgtggttgggtttttggttctaccatgaacaacaagtcacagtcggtgattattattaacaattctactaatgttgttatacgagcatgtaactttga |
| | | attgtgtgacaaccctttctttgctgtttctaaacccatgggtacacagacacatactatgatattcgataatgcatttaattgcactttcgagtacatatctgatgccttttcgcttga |
| | | tgtttcagaaaagtcaggtaattttaaacacttacgagagtttgtgtttaaaaataaagatgggtttctctatgtttataagggctatcaacctatagatgtagttcgtgatctacctt |
| | | ctggttttaacactttgaaacctatttttaagttgcctcttggtattaacattacaaattttagagccattcttacagccttttcacctgctcaagacatttggggcacgtcagctgca |
| | | gcctatt-ttgttggctatttaaagccaactacatttatgctcaagtatgatgaaaatggtacaatcacagatgctgttgattgttctcaaaatccacttgctgaactcaaatgctctg |
| | | ttaagagctttgagattgacaaaggaatttaccagacctctaatttcagggttgttccctcaggagatgttgtgagattccctaatattacaaacttgtgtccttttggagaggtttt |
| | | taatgctactaaattcccttctgtctatgcatgggagagaaaaaaaatttctaattgtgttgctgattactctgtgctctacaactcaacatttttttcaacctttaagtgctatggcgt |
| | | ttctgccactaagttgaatgatctttgcttctccaatgtctatgcagattcttttgtagtcaagggagatgatgtaagacaaatagcgccaggacaaactggtgttattgctgatta |
| | | taattataaattgccagatgatttcatgggttgtgtccttgcttggaatactaggaacattgatgctacttcaactggtaattataattataaatataggtatcttagacatggcaag |
| | | cttaggccattgagagagacatatctaatgtgcctttctcccctgatggcaaaccttgcaccccacctgctcttaattgttattggccattaaatgattatggtttttacaccacta |
| | | ctggcattggctaccaaccttacagagttgtagtactttatttgaacttttaaatgcaccggccacggtttgtggaccaaaattatccactgaccttattaagaaccagtgtgtca |
| | | attttaattttaatggactcactggtactggtgtgttaactccttcttcaaagagatttcaaccatttcaacaatttggccgtgatgtttctgatttcactgattccgttcgagatccta |
| | | aaacatctgaaatattagacatttcaccttgcgcttttgggggtgtaagtgtaattacacctggaacaaatgcttcatctgaagttgctgttctatatcaagatgttaactgcactg |
| | | atgtttctacagcaattcatgcagatcaactcacaccagcttggcgcatatattctactggaaacaatgtattccagactcaagcaggctgtcttataggagctgagcatgtcg |
| | | acacttcttatgagtgcgacattcctattggagaggcatttgtgctagttaccatacagtttattattacgtagtactagccaaaaatctattgtggcttatactatgtattaggtg |
| | | ctgatagttcaattgcttactctaataacaccattgctatacctactaacttttcaattagcattactacagaagtaatgcctgtttctatggctaaaacctccgtagattgtaatatgt |
| | | acatctgcggagattctactgaatgtgctaatttgcttctccaatatggtagcttttgcacacaactaaatcgtgcactctcaggtattgctgctgaacaggatcgcaacacacg |
| | | tgaagtgttcgctcaagtcaaacaaatgtacaaaaccccaactttgaaatattttggtggttttaatttttcacaaatattacctgaccctctaaagccaactaagaggtcttttatt |
| | | gaggacttgctctttaataaggtgacactcgctgatgctggcttcatgaagcaatatggcgaatgcctaggtgatattaatgctagagatctcatttgtgcgcagaagttcaat |
| | | ggacttacagtgttgccacctctgctcactgatgatatgattgctgcctacactgctgctctagttagtggtactgccactgctggatggacatttggtgctggcgctgctcttc |
| | | aaataccttttgctatgcaaatggcatataggttcaatggcattggagttacccaaaatgttctctatgagaaccaaaaacaaatcgccaaccaatttaacaaggcgattagtc |
| | | aaattcaagaatcacttacaacaacatcaactgcattgggcaagctgcaagacgttgttaaccagaatgctcaagcattaaacacacttgttaaacaacttagctctaattttgg |
| | | tgcaatttcaagtgtgctaaatgatatcctttcgcgacttgataaagtcgaggcggaggtacaaattgacaggttaattacaggcagacttcaaagccttcaaacctatgtaac |
| | | acaacaactaatcagggctgctgaaatcagggcttctgctaatcttgctgctactaaaatgtctgagtgtgttcttggacaatcaaaaagagttgacttttgtggaaagggcta |
| | | ccaccttatgtccttcccacaagcagccccgcatggtgttgtcttcctacatgtcacgtatgtgccatcccaggagaggaacttcaccacagcgccagcaatttgtcatgaag |
| | | gcaaagcatacttccctcgtgaaggtgtttttgtgtttaatggcacttcttggtttattacacagaggaacttatttctccacaaataattactacagacaatacatttgtctcagga |
| | | aattgtgatgtcgttattggcatcattaacaacacagtttatgatcctctgcaacctgagcttgactcattcaaagaagagctggacaagtacttcaaaaatcatacatcaccag |
| | | atgttgatcttggcgacatttcaggcattaacgcttctgtcgtcaacattcaaaaagaaattgaccgcctcaatgaggtcgctaaaaatttaaatgaatcactcattgaccttcaa |
| | | gaattgggaaaatatgagcaatatattaaatggccttggtatgtttggctcggcttcattgctggactaattgccatcgtcatggttacaatcttgattgttgcatgactagttgtt |
| | | gcagttgcctcaagggtgcatgctcttgtggttcttgctgcaagtttgatgaggatgactctgagccagttctcaagggtgtcaaattacattacacataaacgaacttatggat |
| | | ttgtttatgagattttttactcttagatcaattactgcacagccagtaaaaattgacaatgcttctcctgcaagtactgttcatgctacagcaacgataccgctacaagcctcactc |
| | | cattcggatggcttgttattggcgttgcatttcttgctgtttttcagagcgctaccaaaataattgcgctcaataaaagatggcagctagccattataagggcttccagttcattt |
| | | gcaatttactgctgctatttgttaccatctattcacatatttgcttgtcgctgcaggtatggaggcgcaatttttgtacctctatgccttgatatattttctacaatgcatcaacgcat |
| | | gtagaattattatgagatgttggctttgttggaagtgcaaatccaagaacccattactttatgatgccaactactttgtttgctggcacacacataactatgactactgtataccat |
| | | ataacagtgtcacagatacaattgtcgttactgaaggtgacggcatttcaacaccaaaactcaaagaagactaccaaattggtggttattctgaggataggcactcaggtgtt |
| | | aaagactatgtcgttgtacatggctatttcaccgaagtttactaccagcttgagtctacacaaattactacagacactggtattgaaaatgctacattcttcatctttaacaagctt |
| | | gttaaagacccaccgaatgtgcaaatacacacaatcgacggctcttcaggagttgctaatccagcaatggatccaatttatgatgagccgacgacgactactagcgtgcctt |
| | | tgtaagcacaagaaagtgagtacgaacttatgtactcattcgtttcggaagaaacaggtacgttaatagttaatagcgtacttctttttcttgattcgtggtattcttgctagtcac |
| | | actagccatccttactgcgcttcgattgtgtgcgtactgctgcaatattgttaacgtgagtttagtaaaaccaacggtttacgtctactcgcgtgttaaaaatctgaactcttctga |
| | | aggagttcctgatcttctggtctaaacgaactaactattattattattctgtttggaactttaacattgcttatcatggcagacaacggtactattaccgttgaggagcttaaacaac |
| | | tcctggaacaatggaacctagtaataggtttcctattcctagcctggattatgttactacaatttgcctattctaatcggaacaggtttttgtacataataaagcttgttttcctctgg |
| | | ctcttgtggccagtaacacttgcttgttttgtgcttgctgctgtctacagaattaattgggtgactggcgggattgcgattgcaatggcttgtattgtaggcttgatgtggcttagc |
| | | tacttcgttgcttccttcaggctgtttgctcgtacccgctcaatgtggtcattcaacccagaaacaaacattcttctcaatgtgcctctccgggggacaattgtgaccagaccgc |
| | | tcatggaaagtgaacttgtcattggtgctgtgatcattcgtggtcacttgcgaatggccggacactccctagggcgctgtgacattaaggacctgccaaaagagatcactgt |
| | | ggctacatcacgaacgattcttattacaaattaggagcgtcgcagcgtgtaggcactgattcaggttttgctgcatacaaccgctaccgtattggaaactataaattaaataca |
| | | gaccacgccggtagcaacgacaatattgattgctagtacagtaagtgacaacagatgtttcatcttgttgacttccaggttacaatagcagagatattgattatcattatgagg |
| | | actttcaggattgctatttggaatcttgacgttataataagttcaatagtgagacaattatttaagcctctaactaagaagaattattcggagttagatgatgaagaacctatggag |
| | | ttagattatccataaaacgaacatgaaaattattctcttcctgacattgattgtatttacatcttgcgagctatatcactatcaggagtgtgttagaggtacgactgtactactaaaa |
| | | gaaccttgcccatcaggaacatacgagggcaattcaccatttcaccctcttgctgacaataaatttgcactaacttgcactagcacacactttgatttgcttgtgctgacggta |
| | | ctcgacatacctatcagctgcgtgcaagatcagtttcaccaaaacttttcatcagacaagaggaggttcaacaagagctctactcgccactttttctcattgttgctgctctagta |
| | | tttttaatactttgcttcaccattaagagaaagacagaatgaatgagctcactttaattgacttctatttgtgctttttagcctttctgctattccttgttttaataatgcttattatattttg |
| | | gttttcactcgaaatccaggatctagaagaaccttgtaccaaagtctaaacgaacatgaaacttctcattgttttgacttgtatttactatgcagttgcatatgcactgtagtacagc |
| | | gctgtgcatctaataaacctcatgtgcttgaagatccttgtaaggtacaacactaggggtaatacttatagcactgcttggctttgtgctctaggaaaggttttaccttttcataga |
| | | tggcacactatggttcaaacatgcacacctaatgttactatcaactgtcaagatccagaggtggtgcgcttatagctaggtgttggtaccttcatgaaggtcaccaaactgct |
| | | gcatttagagacgtacttgttgttttaaataaacgaacaaattaaaatgtctgataatggaccccaatcaaaccaacgtagtgccccccgcattacatttggtggacccacaga |
| | | ttcaactgacaataaccagaatggaggacgcaatggggcaaggccaaaacagcgccgaccccaaggtttacccaataatactgcgtcttggttcacagctctcactcagc |
| | | atggcaaggaggaacttagattccctcgaggccagggcgttccaatcaacaccaatagtggtccagatgaccaaattggctactaccgaagagctacccgacgagttcgt |
| | | ggtggtgacggcaaaatgaaagagctcagccccagatggtacttctattacctaggaactggcccagaagcttcacttccctacggcgctaacaaagaaggcatcgtatg |
| | | ggttgcaactgagggagccttgaatacacccaaagaccacattggcacccgcaatcctaataacaatgctgccaccgtgctacaacttcctcaaggaacaacattgccaa |
| | | aaggatctacgcagagggaagcagaggcggcagtcaagcctcttctcgctcctcatcacgtagtcgcggtaattcaagaaattcaactcctggcagcagtaggggaaat |
| | | tctcctgctcgaatggctagcggaggtggtgaaactgccctcgcgctattgctgctagacagattgaaccagcttgagagcaaagtttctggtaaaggccaacaacaacaa |
| | | ggccaaactgtcactaagaaatctgctgctgaggcatctaaaaagcctcgccaaaaacgtactgccacaaaacagtacaacgtcactcaagcatttgggagacgtggtcc |
| | | agaacaaacccaaggaaatttcggggaccaagacctaatcagacaaggaactgattacaaacattggccgcaaattgcacaatttgctccaagtgcctctgcattctttgga |
| | | atgtcacgcattggcatggaagtcacaccttcgggaacatggctgacttatcatggagccattaaattggatgacaaagatccacaattcaaagacaacgtcatactgctga |
| | | acaagcacattgacgcatacaaaacattcccaccaacagagcctaaaaaggacaaaaagaaaaagactgatgaagctcagcctttgccgcagagacaaaagaagcag |
| | | cccactgtgactatatcctgcggctgacatggatgatttctccagacaacttcaaaattccatgagtggagcttctgctgattcaactcaggcataaacactcatgatgacca |
| | | cacaaggcagatgggctatgtaaacgttttcgcaattccgtttacgatacatagtctactcttgtgcagaatgaattctcgtaactaaacagcacaagtaggtttagttaacttta |
| | | atctcacatagcaatctttaatcaatgtgtaacattagggaggacttgaaagagccaccacattttcatcgaggccacgcggagtacgatcgagggtacagtgaataatgct |
| | | agggagagctgcctatatggaagagccctaatgtgtaaaattaattttagtagtgctatccccatgtgattttaatagcttcttaggagaatgacaaaaaaaaaaaaaaaaaaa |
| | | aaaaa |
| 2409 | MERS-CoV | gatttaagtgaatagcttggctatctcacttcccctcgttctcttgcagaactttgattttaacgaacttaaataaaagccctgttgtttagcgtatcgttgcacttgtctggtgggat |
| | genome | tgtggcattaatttgcctgctcatctaggcagtggacatatgctcaacactgggtataattctaattgaatactatttttcagttagagcgtcgtgtctcttgtacgtctcggtcaca |
| | (Genbank | atacacggtttcgtccggtgcgtggcaattcggggcacatcatgtctttcgtggctggtgtgaccgcgcaaggtgcgcgcggtacgtatcgagcagcgctcaactctgaaa |
| | Accession | aacatcaagaccatgtgtctctaactgtgccactctgtggttcaggaaacctggttgaaaaactttcaccatggttcatggatggcgaaaatgcctatgaagtggtgaaggcc |
| | No. | atgttacttaaaaaggagccacttctctatgtgcccatccggctggctggacacactagacacctcccaggtcctcgtgtgtacctggttgagaggctcattgcttgtgaaaat |
| | NC_ | ccattcatggttaaccaattggcttatagctctagtgcaaatggcagcctggttggcacaactttgcagggcaagcctattggtatgttcttcccttatgacatcgaacttgtcac |
| | 019843.3) | aggaaagcaaaatattctcctgcgcaagtatggccgtggtggttatcactacaccccattccactatgagcgagacaacacctcttgccctgagtggatggacgattttgag |
| | | gcggatcctaaaggcaaatatgcccagaatctgcttaagaagttgattggcggtgatgtcactccagttgaccaatacatgtgtggcgttgatggaaaacccattagtgccta |
| | | cgcatttttaatggccaaggatggaataaccaaactggctgatgttgaagcggacgtcgcagcacgtgctgatgacgaaggcttcatcacattaaagaacaatctatataga |
| | | ttggtttggcatgttgagcgtaaagacgttccatatcctaagcaatctatttttactattaatagtgtggtccaaaaggatggtgttgaaaacactcctcctcactattttactcttgg |
| | | atgcaaaattttaacgctcaccccacgcaacaagtggagtggcgtttctgacttgtccctcaaacaaaaactcctttacaccttctatggtaaggagtcacttgagaacccaac |
| | | ctacatttaccactccgcattcattgagtgtggaagttgtggtaatgattcctggcttacagggaatgctatccaagggtttgcctgtggatgtggggcatcatatacagctaat |
| | | gatgtcgaagtccaatcatctggcatgattaagccaaatgctcttctttgtgctacttgcccctttgctaagggtgatagctgttcttctaattgcaaacattcagttgctcagttgg |
| | | ttagttacctttctgaacgctgtaatgttattgctgattctaagtccttcacacttatattggtggcgtagcttacgcctactttggatgtgaggaaggtactatgtactttgtgccta |
| | | gagctaagtctgttgtctcaaggattggagactccatctttacaggctgtactggctcttggaacaaggtcactcaaattgctaacatgttcttggaacagactcagcattccct |
| | | taactttgtgggagagttcgttgtcaacgatgttgtcctcgcaattctctctggaaccacaactaatgttgacaaaatacgccagcttctcaaaggtgtcacccttgacaagttg |
| | | cgtgattatttagctgactatgacgtagcagtcactgccggcccattcatggataatgctattaatgttggtggtacaggattacagtatgccgccattactgcaccttatgtagt |
| | | tctcactggcttaggtgagtcctttaagaaagttgcaaccataccgtataaggtttgcaactctgttaaggatactctggcttattatgctcacagcgtgttgtacagagtttttcct |
| | | tatgacatggattctggtgtgtcatcctttagtgaactactttttgattgcgttgatattcagtagcttctacctattttttagtccgcatcttgcaagataagactggcgactttatgt |
| | | ctacaattattacttcctgccaaactgctgttagtaagcttctagatacatgttttgaagctacagaagcaacatttaacttcttgttagatttggcaggattgttcagaatctttctcc |
| | | gcaatgcctatgtgtacacttcacaagggtttgtggtggtcaatggcaaagtttctacacttgtcaaacaagtgttagacttgcttaataagggtatgcaacttttgcatacaaag |
| | | gtctcctgggctggttctaaaatcattgctgttatctacagcggcagggagtctctaatattcccatcgggaacctattactgtgtcaccactaaggctaagtccgttcaacaag |
| | | atcttgacgttatt-ttgcctggtgagttttccaagaagcagttaggactgctccaacctactgacaattctacaactgttagtgttactgtatccagtaacatggttgaaactgttgt |
| | | gggtcaacttgagcaaactaatatgcatagtcctgatgttatagtaggtgactatgtcattattagtgaaaaattgtttgtgcgtagtaaggaagaagacggatttgccttctacc |
| | | ctgcttgcactaatggtcatgctgtaccgactctctttagacttaagggaggtgcacctgtaaaaaaagtagcctttggcggtgatcaagtacatgaggttgctgctgtaagaa |
| | | gtgttactgtcgagtacaacattcatgctgtattagacacactacttgcttcttctagtcttagaacctttgttgtagataagtctttgtcaattgaggagtttgctgacgtagtaaag |
| | | gaacaagtctcagacttgcttgttaaattactgcgtggaatgccgattccagattttgatttagacgattttattgacgcaccatgctattgctttaacgctgagggtgatgcatcc |
| | | tggtcttctactatgatcttctctcttcaccccgtcgagtgtgacgaggagtgttctgaagtagaggcttcagatttagaagaaggtgaatcagagtgcatttctgagacttcaa |
| | | ctgaacaagttgacgtttctcatgagacttctgacgacgagtgggctgctgcagttgatgaagcgttccctctcgatgaagcagaagatgttactgaatctgtgcaagaaga |
| | | agcacaaccagtagaagtacctgttgaagatattgcgcaggttgtcatagctgacaccttacaggaaactcctgttgtgcctgatactgttgaagtcccaccgcaagtggtg |
| | | aaacttccgtctgcacctcagactatccagcccgaggtaaaagaagttgcacctgtctatgaggctgataccgaacagacacagaatgttactgttaaacctaagaggttac |
| | | gcaaaaagcgtaatgttgaccattgtccaattttgaacataaggttattacagagtgcgttaccatagttttaggtgacgcaattcaagtagccaagtgctatggggagtctgt |
| | | gttagttaatgctgctaacacacatcttaagcatggcggtggtatcgctggtgctattaatgcggcttcaaaaggggctgtccaaaaagagtcagatgagtatattctggctaa |
| | | agggccgttacaagtaggagattcagttctcttgcaaggccattctctagctaagaatatcctgcatgtcgtaggcccagatgcccgcgctaaacaggatgtttctctccttag |
| | | taagtgctataaggctatgaatgcatatcctcttgtagtcactcctcttgtttcagcaggcatatttggtgtaaaaccagctgtgtcttttgattatcttattagggaggctaagact |
| | | agagttttagtcgtcgttaattcccaagatgtctataagagtcttaccatagttgacattccacagagtttgactttttcatatgatgggttacgtggcgcaatacgtaaagctaaa |
| | | gattatggttttactgtttttgtgtgcacagacaactctgctaacactaaagttcttaggaacaagggtgttgattatactaagaagtttcttacagttgacggtgtgcaatattattg |
| | | ctacacgtctaaggacactttagatgatatcttacaacaggctaataagtagttggtattatatctatgcctttgggatatgtgtctcatggtttagacttaatgcaagcagggag |
| | | tgtcgtgcgtagagttaacgtgccctacgtgtgtctcctagctaataaagagcaagaagctattttgatgtctgaagacgttaagttaaacccttcagaagattttataaagcac |
| | | gtccgcactaatggtggttacaattcttggcatttagtcgagggtgaactattggtgcaagacttacgcttaaataagctcctgcattggtctgatcaaaccatatgctacaagg |
| | | atagtgtgttttatgttgtaaagaatagtacagcttttccatttgaaacactttcagcatgtcgtgcgtatttggattcacgcacgacacagcagttaacaatcgaagtcttagtga |
| | | ctgtcgatggtgtaaattttagaacagtcgttctaaataataagaacacttatagatcacagcttggatgcgttttattaatggtgctgatatttctgacaccattcctgatgagaa |
| | | acagaatggtcacagtttatatctagcagacaatttgactgctgatgaaacaaaggcgcttaaagagttatatggccccgttgatcctactttcttacacagattctattcactta |
| | | aggctgcagtccatgggtggaagatggttgtgtgtgataaggtacgttctctcaaattgagtgataataattgttatcttaatgcagttattatgacacttgatttattgaaggaca |
| | | ttaaatttgttatacctgactacagcatgcatttatgaaacataagggcggtgattcaactgacttcatagccctcattatggcttatggcaattgcacatttggtgaccagatg |
| | | atgcctctcggttacttcataccgtgcttgcaaaggctgagttatgctgttctgcacgcatggtttggagagagtggtgcaatgtctgtggcataaaagatgttgttctacaagg |
| | | cttaaaagcttgttgttacgtgggtgtgcaaactgttgaagatctgcgtgctcgcatgacatatgtatgccagtgtggtggtgaacgtcatcggcaattagtcgaacacacca |
| | | ccccctggttgctgctctcaggcacaccaaatgaaaaattggtgacaacctccacggcgcctgattttgtagcatttaatgtctttcagggcattgaaacggctgttggccatt |
| | | atgttcatgctcgcctgaagggtggtcttattttaaagtttgactaggcaccgttagcaagacttcagactggaagtgcaaggtgacagatgtacttttccccggccaaaaat |
| | | acagtagcgattgtaatgtcgtacggtattctttggacggtaatttcagaacagaggttgatcccgacctatctgctttctatgttaaggatggtaaatactttacaagtgaacca |
| | | cccgtaacatattcaccagctacaattttagctggtagtgtctacactaatagctgccttgtatcgtctgatggacaacctggcggtgatgctattagtttgagttttaataaccttt |
| | | tagggtttgattctagtaaaccagtcactaagaaatacacttactccttcttgcctaaagaagacggcgatgtgttgttggctgagtttgacacttatgaccctatttataagaatg |
| | | gtgccatgtataaaggcaaaccaattctttgggtcaataaagcatcttatgatactaatcttaataagttcaatagagctagtttgcgtcaaatttttgacgtagcccccattgaac |
| | | tcgaaaataaattcacacctttgagtgtggagtctacaccagttgaacctccaactgtagatgtggtagcacttcaacaggaaatgacaattgtcaaatgtaagggtttaaata |
| | | aacctttcgtgaaggacaatgtcagtttcgttgctgatgattcaggtactcccgttgttgagtatctgtctaaagaagacctacatacattgtatgtagaccctaagtatcaagtc |
| | | attgtcttaaaagacaatgtactttcttctatgcttagattgcacaccgttgagtcaggtgatattaacgttgttgcagcttccggatctttgacacgtaaagtgaagttactattta |
| | | gggcttcattttatttcaaagaatttgctacccgcactttcactgctaccactgctgtaggtagttgtataaagagtgtagtgcggcatctaggtgttactaaaggcatattgaca |
| | | ggctgttttagttttgccaagatgttatttatgcttccactagcttactttagtgattcaaaactcggcaccacagaggttaaagtgagtgattgaaaacagccggcgttgtgac |
| | | aggtaatgttgtaaaacagtgttgcactgctgctgttgatttaagtatggataagttgcgccgtgtggattggaaatcaaccctacggttgttacttatgttatgcacaactatggt |
| | | attgttgtcttctgtgtatcacttgtatgtcttcaatcaggtcttatcaagtgatgttatgtttgaagatgcccaaggtttgaaaaagttctacaaagaagttagagcttacctagga |
| | | atctcttctgcttgtgacggtcttgcttcagcttatagggcgaattcctttgatgtacctacattctgcgcaaaccgttctgcaatgtgtaattggtgcttgattagccaagattcca |
| | | taactcactacccagctcttaagatggttcaaacacatcttagccactatgttcttaacatagattggttgtggtttgcatttgagactggtttggcatacatgctctatacctcggc |
| | | cttcaactggttgttgttggcaggtacattgcattatttctttgcacagacttccatatttgtagactggcggtcatacaattatgctgtgtctagtgccttctggttattcacccacat |
| | | tccaatggcgggtttggtacgaatgtataatttgttagcatgcctttggcttttacgcaagttttatcagcatgtaatcaatggttgcaaagatacggcatgcttgctctgctataa |
| | | gaggaaccgacttactagagttgaagcttctaccgttgtagtggtggaaaacgtacgttttatatcacagcaaatggcggtatttcattctgtcgtaggcataattggaattgt |
| | | gtggattgtgacactgcaggtgtggggaataccttcatctgtgaagaagtcgcaaatgacctcactaccgccctacgcaggcctattaacgctacggatagatcacattatta |
| | | tgtggattccgttacagttaaagagactgttgttcagtttaattatcgtagagacggtcaaccattctacgagcggtttcccctctgcgcttttacaaatctagataagttgaagtt |
| | | caaagaggtagtaaaactactactggtatacctgaatacaactttatcatctacgactcatcagatcgtggccaggaaagtttagctaggtctgcatgtgtttattattctcaagt |
| | | cttgtgtaaatcaattcttttggttgactcaagtttggttacttctgttggtgattctagtgaaatcgccactaaaatgtttgattcctttgttaatagtttcgtctcgctgtataatgtca |
| | | cacgcgataagttggaaaaacttatctctactgctcgtgatggcgtaaggcgaggcgataacttccatagtgtcttaacaacattcattgacgcagcacgaggccccgcag |
| | | gtgtggagtctgatgttgagaccaatgaaattgttgactctgtgcagtatgctcataaacatgacatacaaattactaatgagagctacaataattatgtaccctcatatgttaaa |
| | | cctgatagtgtgtctaccagcgatttaggtagtctcattgattgtaatgcggcttcagttaaccaaattgtcttgcgtaattctaatggtgcttgcatttggaacgctgctgcatata |
| | | tgaaactctcggatgcacttaaacgacagattcgcattgcatgccgtaagtgtaatttagctttccggttaaccacctcaaagctacgcgctaatgataatatcttatcagttaga |
| | | ttcactgctaacaaaattgttggtggtgctcctacatggtttaatgcgttgcgtgactttacgttaaagggttatgttcttgctaccattattgtgtttctgtgtgctgtactgatgtatt |
| | | tgtgtttacctacattttctatggcacctgttgaattttatgaagaccgcatcttggactttaaagttcttgataatggtatcattagggatgtaaatcctgatgataagtgattgcta |
| | | ataagcaccggtccttcacacaatggtatcatgagcatgttggtggtgtctatgacaactctatcacatgcccattgacagttgcagtaattgctggagttgctggtgctcgcat |
| | | tccagacgtacctactacattggcttgggtgaacaatcagataattttattgtttctcgagtctttgctaatacaggcagtgtttgctacactcctatagatgagataccctataag |
| | | agtttctctgatagtggttgcattcttccatctgagtgcactatgtttagggatgcagagggccgtatgacaccatactgccatgatcctactgttttgcctggggcttttgcgtac |
| | | agtcagatgaggcctcatgttcgttacgacttgtatgatggtaacatgtttattaaatttcctgaagtagtatttgaaagtacacttaggattactagaactctgtcaactcagtact |
| | | gccggttcggtagttgtgagtatgcacaagagggtgtttgtattaccacaaatggctcgtgggccatttttaatgaccaccatcttaatagacctggtgtctattgtggctctgat |
| | | tttattgacattgtcaggcggttagcagtatcactgttccagcctattacttatttccaattgactacctcattggtcttgggtataggtttgtgtgcgttcctgactttgctcttctatt |
| | | atattaataaagtaaaacgtgatttgcagattacacccagtgtgctgtaattgctgttgttgctgctgttcttaatagcttgtgcatctgctttgttacctctataccattgtgtatagt |
| | | accttacactgcattgtactattatgctacattctattttactaatgagcctgcatttattatgcatgtttcttggtacattatgttcgggcctatcgttcccatatggatgacctgcgtc |
| | | tatacagttgcaatgtgattagacacttcttctgggttttagcttattttagtaagaaacatgtagaagtttttactgatggtaagcttaattgtagtttccaggacgctgcctctaat |
| | | atctttgttattaacaaggacacttatgcagctcttagaaactctttaactaatgatgcctattcacgatttttggggttgtttaacaagtataagtacttctctggtgctatggaaac |
| | | agccgcttatcgtgaagctgcagcatgtcatcttgctaaagccttacaaacatacagcgagactggtagtgatcttctttaccaaccacccaactgtagcataacctaggcgt |
| | | gttgcaaagcggtttggtgaaaatgtcacatcccagtggagatgttgaggcttgtatggttcaggttacctgcggtagcatgactcttaatggtctttggcttgacaacacagt |
| | | ctggtgcccacgacacgtaatgtgcccggctgaccagttgtctgatcctaattatgatgccttgttgatttctatgactaatcatagtttcagtgtgcaaaaacacattggcgctc |
| | | cagcaaacttgcgtgttgttggtcatgccatgcaaggcactcttttgaagttgactgtcgatgttgctaaccctagcactccagcctacacttttacaacagtgaaacctggcg |
| | | cagcatttagtgtgttagcatgctataatggtcgtccgactggtacattcactgttgtaatgcgccctaactacacaattaagggttcctttctgtgtggttcttgtggtagtgttgg |
| | | ttacaccaaggagggtagtgtgatcaatttctgttacatgcatcaaatggaacttgctaatggtacacataccggttcagcatttgatggtactatgtatggtgcctttatggata |
| | | aacaagtgcaccaagttcagttaacagacaaatactgcagtgttaatgtagtagcttggctttacgcagcaatacttaatggttgcgcttggtttgtaaaacctaatcgcactag |
| | | tgttgtttcttttaatgaatgggctcttgccaaccaattcactgaatttgttggcactcaatccgttgacatgttagctgtcaaaacaggcgttgctattgaacagctgattatgcg |
| | | atccaacaactgtatactgggttccagggaaagcaaatccttggcagtaccatgttggaagatgaattcacacctgaggatgttaatatgcagattatgggtgtggttatgca |
| | | gagtggtgtgagaaaagttacatatggtactgcgcattggttgtttgcgacccttgtctcaacctatgtgataatcttacaagccactaaatttactttgtggaactacttgtttga |
| | | gactattcccacacagttgttcccactcttatttgtgactatggccttcgttatgttgttggttaaacacaaacacacctttttgacacttttcttgttgcctgtggctatttgtttgactt |
| | | atgcaaacatagtctacgagcccactactcccatttcgtcagcgctgattgcagttgcaaattggcttgcccccactaatgcttatatgcgcactacacatactgatattggtgt |
| | | ctacattagtatgtcacttgtattagtcattgtagtgaagagattgtacaacccatcactttctaactttgcgttagcattgtgcagtggtgtaatgtggttgtacacttatagcattg |
| | | gagaagcctcaagccccattgcctatctggtttttgtcactacactcactagtgattatacgattacagtctttgttactgtcaaccttgcaaaagtttgcacttatgccatctttgct |
| | | tactcaccacagcttacacttgtgtttccggaagtgaagatgatacttttattatacacatgtttaggtttcatgtgtacttgctattttggtgtcttctctcttttgaaccttaagcttag |
| | | agcacctatgggtgtctatgactttaaggtctcaacacaagagttcagattcatgactgctaacaatctaactgcacctagaaattcttgggaggctatggctctgaactttaag |
| | | ttaataggtattggcggtacaccttgtataaaggttgctgctatgcagtctaaacttacagatcttaaatgcacatctgtggttctcctctctgtgctccaacagttacacttagag |
| | | gctaatagtagggcctgggctttctgtgttaaatgccataatgatatattggcagcaacagaccccagtgaggctttcgagaaattcgtaagtctctttgctactttaatgactttt |
| | | tctggtaatgtagatcttgatgcgttagctagtgatatttttgacactcctagcgtacttcaagctactctttctgagttttcacacttagctacctttgctgagttggaagctgcgca |
| | | gaaagcctatcaggaagctatggactaggtgacacctcaccacaagttcttaaggctttgcagaaggctgttaatatagctaaaaacgcctatgagaaggataaggcagt |
| | | ggcccgtaagttagaacgtatggctgatcaggctatgacttctatgtataagcaagcacgtgctgaagacaagaaagcaaaaattgtcagtgctatgcaaactatgttgtttg |
| | | gtatgattaagaagctcgacaacgatgttcttaatggtatcatttctaacgctaggaatggttgtatacctcttagtgtcatcccactgtgtgcttcaaataaacttcgcgttgtaat |
| | | tcctgacttcaccgtaggaatcaggtagtcacatatccctcgcttaactacgctggggctttgtgggacattacagttataaacaatgtggacaatgaaattgttaagtcttca |
| | | gatgttgtagacagcaatgaaaatttaacatggccacttgttttagaatgcactagggcatccacttctgccgttaagttgcaaaataatgagatcaaaccttcaggtctaaaaa |
| | | ccatggttgtgtctgcgggtcaagagcaaactaactgtaatactagttccttagcttattacgaacctgtgcagggtcgtaaaatgctgatggctcttctttctgataatgcctat |
| | | ctcaaatgggcgcgtgttgaaggtaaggacggatttgtcagtgtagagctacaacctccttgcaaattcttgattgcgggaccaaaaggacctgaaatccgatatctctatttt |
| | | gttaaaaatcttaacaaccttcatcgcgggcaagtgttagggcacattgctgcgactgttagattgcaagctggttctaacaccgagtttgcctctaattcctcggtgttgtcact |
| | | tgttaacttcaccgttgatcctcaaaaagcttatctcgatttcgtcaatgcgggaggtgccccattgacaaattgtgttaagatgcttactcctaaaactggtacaggtatagcta |
| | | tatctgttaaaccagagagtacagctgatcaagagacttatggtggagcttcagtgtgtactattgccgtgcgcatatagaacatcctgatgtactggtgtttgtaaatataag |
| | | ggtaagtttgtccaaatccctgctcagtgtgtccgtgaccctgtgggattttgtttgtcaaataccccctgtaatgtctgtcaatattggattggatatgggtgcaattgtgactcg |
| | | cttaggcaagcagcactgccccaatctaaagattccaattttttaaacgagtccggggttctattgtaaatgcccgaatagaaccctgttcaagtggtttgtccactgatgtcgt |
| | | ctttagggcatttgacatctgcaactataaggctaaggttgctggtattggaaaatactacaagactaatacttgtaggtttgtagaattagatgaccaagggcatcatttagact |
| | | cctattttgtcgttaagaggcatactatggagaattatgaactagagaagcactgttacgacttgttacgtgactgtgatgctgtagctccccatgatttcttcatctttgatgtag |
| | | acaaagttaaaacacctcatattgtacgtcagcgtttaactgagtacactatgatggatcttgtatatgccctgaggcactttgatcaaaatagcgaagtgcttaaggctatctta |
| | | gtgaagtatggttgctgtgatgttacctactttgaaaataaactctggtttgattttgttgaaaatcccagtgttattggtgtttatcataaacttggagaacgtgtacgccaagcta |
| | | tcttaaacactgttaaattttgtgaccacatggtcaaggctggtttagtcggtgtgctcacactagacaaccaggaccttaatggcaagtggtatgattttggtgacttcgtaatc |
| | | actcaacctggttcaggagtagctatagttgatagctactattcttatttgatgcctgtgctctcaatgaccgattgtctggccgctgagacacatagggattgtgattttaataaa |
| | | ccactcattgagtggccacttactgagtatgattttactgattataaggtacaactattgagaagtactttaaatattgggatcagacgtatcacgcaaattgcgttaattgtactg |
| | | atgaccgttgtgtgttacattgtgctaatttcaatgtattgtttgctatgaccatgcctaagacttgtttcggacccatagtccgaaagatctttgttgatggcgtgccatttgtagta |
| | | tcttgtggttatcactacaaagaattaggtttagtcatgaatatggatgttagtaccatagacataggctctctcttaaggagttgatgatgtatgccgctgatccagccatgca |
| | | cattgcctcctctaacgcttttcttgatttgaggacatcatgttttagtgtcgctgcacttacaactggtttgacttttcaaactgtgcggcctggcaattttaaccaagacttctatg |
| | | atttcgtggtatctaaaggtttctttaaggagggctcttcagtgacgctcaaacattttttctttgctcaagatggtaatgctgctattacagattataattactattcttataatctgcc |
| | | tactatgtgtgacatcaaacaaatgttgttctgcatggaagttgtaaacaagtacttcgaaatctatgacggtggttgtcttaatgcttctgaagtggttgttaataatttagacaa |
| | | gagtgctggccatccttttaataagtttggcaaagctcgtgtctattatgagagcatgtcttaccaggagcaagatgaactttttgccatgacaaagcgtaacgtcattcctacc |
| | | atgactcaaatgaatctaaaatatgctattagtgctaagaatagagctcgcactgttgcaggcgtgtccatacttagcacaatgactaatcgccagtaccatcagaaaatgctt |
| | | aagtccatggctgcaactcgtggagcgacttgcgtcattggtactacaaagttctacggtggctgggatttcatgcttaaaacattgtacaaagatgttgataatccgcatctta |
| | | tgggttgggattaccctaagtgtgatagagctatgcctaatatgtgtagaatcttcgcttcactcatattagctcgtaaacatggcacttgttgtactacaagggacagattttatc |
| | | gcttggcaaatgagtgtgctcaggtgctaagcgaatatgttctatgtggtggtggttactacgtcaaacctggaggtaccagtagcggagatgccaccactgcatatgccaa |
| | | tagtgtctttaacattttgcaggcgacaactgctaatgtcagtgcacttatgggtgctaatggcaacaagattgttgacaaagaagttaaagacatgcagtttgatttgtatgtca |
| | | atgtttacaggagcactagcccagaccccaaatttgttgataaatactatgatttcttaataagcacttttctatgatgatactgtctgatgacggtgtcgtttgctataatagtgat |
| | | tatgcagctaagggttacattgctggaatacagaattttaaggaaacgctgtattatcagaacaatgtctttatgtctgaagctaaatgctgggtggaaaccgatctgaagaaa |
| | | gggccacatgaattctgttcacagcatacgattatattaaggatggcgacgatggttacttccttccttatccagacccttcaagaattttgtctgccggttgattgtagatgat |
| | | atcgttaagactgacggtacactcatggtagagcggtttgtgtctttggctatagatgcttaccctctcacaaagcatgaagatatagaataccagaatgtattctgggtctactt |
| | | acagtatatagaaaaactgtataaagaccttacaggacacatgcttgacagttattctgtcatgctatgtggtgataattctgctaagttttgggaagaggcattctatagagatc |
| | | tctatagttcgcctaccactttgcaggctgtcggttcatgcgttgtatgccattcacagacttccctacgctgtgggacatgcatccgtagaccatttctctgctgtaaatgctgc |
| | | tatgatcatgttatagcaactccacataagatggttttgtctgtttctccttacgtttgtaatgcccctggttgtggcgtttcagacgttactaagctatatttaggtggtatgagcta |
| | | cttttgtgtagatcatagacctgtgtgtagttttccactttgcgctaatggtcttgtattcggcttatacaagaatatgtgcacaggtagtccttctatagttgaatttaataggttggc |
| | | tacctgtgactggactgaaagtggtgattacacccttgccaatactacaacagaaccactcaaactttttgctgctgagactttacgtgccactgaagaggcgtctaagcagt |
| | | cttatgctattgccaccatcaaagaaattgttggtgagcgccaactattacttgtgtgggaggctggcaagtccaaaccaccactcaatcgtaattatgtttttactggttatcat |
| | | ataaccaaaaatagtaaagtgcagctcggtgagtacattttcgagcgcattgattatagtgatgctgtatcctacaagtctagtacaacgtataaactgactgtaggtgacatct |
| | | tcgtacttacctctcactctgtggctaccttgacggcgcccacaattgtgaatcaagagaggtatgttaaaattactgggttgtacccaaccattacggtacctgaagagttcg |
| | | caagtcatgttgccaacttccaaaaatcaggttatagtaaatatgtcactgttcagggaccacctggcactggcaaaagtcattttgctatagggttagcgatttactaccctac |
| | | agcacgtgttgtttatacagcatgttcacacgcagctgttgatgattgtgtgaaaaagcttttaaatatttgaacattgctaaatgttcccgtatcattcctgcaaaggcacgtgtt |
| | | gagtgctatgacaggtttaaagttaatgagacaaattctcaatatttgtttagtactattaatgactaccagaaacttctgccgatattctggtggttgatgaggttagtatgtgca |
| | | ctaattatgatattcaattattaatgcacgtattaaagctaagcacattgtctatgtaggagatccagcacagttgccagctcctaggactttgttgactagaggcacattggaa |
| | | ccagaaaatttcaatagtgtcactagattgatgtgtaacttaggtcctgacatatttttaagtatgtgctacaggtgtcctaaggaaatagtaagcactgtgagcgctcttgtcta |
| | | caataataaattgttagccaagaaggagctttcaggccagtgctttaaaatactctataagggcaatgtgacgcatgatgctagctctgccattaatagaccacaactcacatt |
| | | tgtgaagaattttattactgccaatccggcatggagtaaggcagtctttatttcgccttacaattcacagaatgctgtgtctcgttcaatgctgggtcttaccactcagactgttga |
| | | ttcctcacagggttcagaataccagtacgttatcttctgtcaaacagcagatacggcacatgctaacaacattaacagatttaatgttgcaatcactcgtgcccaaaaaggtatt |
| | | ctttgtgttatgacatctcaggcactctttgagtccttagagtttactgaattgtatttactaattacaagctccagtctcagattgtaactggcctttttaaagattgctctagagaa |
| | | acttctggcctctcacctgcttatgcaccaacatatgttagtgttgatgacaagtataagacgagtgatgagctttgcgtgaatcttaatttacccgcaaatgtcccatactctcgt |
| | | gttatttccaggatgggctttaaactcgatgcaacagttcctggatatcctaagcttttcattactcgtgaagaggctgtaaggcaagttcgaagctggataggcttcgatgttg |
| | | agggtgctcatgcttcccgtaatgcatgtggcaccaatgtgcctctacaattaggattttcaactggtgtgaactttgttgttcagccagttggtgttgtagacactgagtgggg |
| | | taacatgttaacgggcattgctgcacgtcctccaccaggtgaacagtttaagcacctcgtgcctcttatgcataagggggctgcgtggcctattgttagacgacgtatagtgc |
| | | aaatgttgtcagacactttagacaaattgtctgattactgtacgtttgtttgttgggctcatggctttgaattaacgtctgcatcatacttttgcaagataggtaaggaacagaagt |
| | | gttgcatgtgcaatagacgcgctgcagcgtactcttcacctctgcaatcttatgcctgctggactcattcctgcggttatgattatgtctacaaccctttctttgtcgatgttcaaca |
| | | gtggggttatgtaggcaatcttgctactaatcacgatcgttattgctctgtccatcaaggagctcatgtggcttctaatgatgcaataatgactcgttgtttagctattcattcttgtt |
| | | ttatagaacgtgtggattgggatatagagtatccttatatctcacatgaaaagaaattgaattcctgttgtagaatcgttgagcgcaacgtcgtacgtgctgctcttcttgccggt |
| | | tcatttgacaaagtctatgatattggcaatcctaaaggaattcctattgttgatgaccagtggttgattggcattattttgatgcacagcccttgaccaggaaggtacaacagct |
| | | tttctatacagaggacatggcctcaagatttgctgatgggctctgcttattttggaactgtaatgtaccaaaatatcctaataatgcaattgtatgcaggtttgacacacgtgtgc |
| | | attctgagttcaatttgccaggttgtgatggcggtagtttgtatgttaacaagcacgcttttcatacaccagcatatgatgtgagtgcattccgtgatctgaaacctttaccattctt |
| | | ttattattctactacaccatgtgaagtgcatggtaatggtagtatgatagaggatattgattatgtacccctaaaatctgcagtagtattacagcttgtaatttagggggcgctgtt |
| | | tgtaggaagcatgctacagagtacagagagtatatggaagcatataatcttgtactgcatcaggtttccgcctttggtgttataagacctttgatatttataatctctggtctactt |
| | | ttacaaaagttcaaggtttggaaaacattgatttaatgttgttaaacaaggccattttattggtgttgagggtgaactacctgtagctgtagtcaatgataagatcttcaccaaga |
| | | gtggcgttaatgacatttgtatgtttgagaataaaaccactttgcctactaatatagcttttgaactctatgctaagcgtgctgtacgctcgcatcccgatttcaaattgctacaca |
| | | atttacaagcagacatttgctacaagttcgtcctttgggattatgaacgtagcaatatttatggtactgctactattggtgtatgtaagtacactgatattgatgttaattcagctttg |
| | | aatatatgttttgacatacgcgataattgttcattggagaagttcatgtctactcccaatgccatctttatttctgatagaaaaatcaagaaatacccttgtatggtaggtcctgatta |
| | | tgcttacttcaatggtgctatcatccgtgatagtgatgttgttaaacaaccagtgaagttctacttgtataagaaagtcaataatgagtttattgatcctactgagtgtatttacactc |
| | | agagtcgctcttgtagtgacttcctacccattctgacatggagaaagactttctatcttttgatagtgatgttttcattaagaagtatggcttggaaaactatgatttgagcacgt |
| | | agtctatggagacttctctcatactacgttaggcggtcttcacttgcttattggtttatacaagaagcaacaggaaggtcatattattatggaagaaatgctaaaaggtagctca |
| | | actattcataactattttattactgagactaacacagcggcttttaaggcggtgtgttctgttatagatttaaagcttgacgactttgttatgattttaaagagtcaagaccttggcgt |
| | | agtatccaaggttgtcaaggttcctattgacttaacaatgattgagtttatgttatggtgtaaggatggacaggttcaaaccttctaccctcgactccaggcttctgcagattgga |
| | | aacctggtcatgcaatgccatccctctttaaagttcaaaatgtaaaccttgaacgttgtgagcttgctaattacaagcaatctattcctatgcctcgcggtgtgcacatgaacatc |
| | | gctaaatatatgcaattgtgccagtatttaaatacttgcacattagccgtgcctgccaatatgcgtgttatacattttggcgctggttctgataaaggtatcgctcctggtacctca |
| | | gttttacgacagtggcttcctacagatgccattattatagataatgatttaaatgagttcgtgtcagatgctgacataactttatttggagattgtgtaactgtacgtgtcggccaa |
| | | caagtggatcttgttatttccgacatgtatgatcctactactaagaatgtaacaggtagtaatgagtcaaaggctttattctttacttacctgtgtaacctcattaataataatcttgct |
| | | cttggtgggtctgttgctattaaaataacagaacactcttggagcgttgaactttatgaacttatgggaaaatttgcttggtggactgttttctgcaccaatgcaaatgcatcctca |
| | | tctgaaggattcctcttaggtattaattacttgggtactattaaagaaaatatagatggtggtgctatgcacgccaactatatattttggagaaattccactcctatgaatctgagta |
| | | cttactcactttttgatttatccaagtttcaattaaaattaaaaggaacaccagttcttcaattaaaggagagtcaaattaacgaactcgtaatatctctcctgtcgcagggtaagtt |
| | | acttatccgtgacaatgatacactcagtgtttctactgatgttcttgttaacacctacagaaagttacgttgatgtagggccagattctgttaagtagcttgtattgaggttgatat |
| | | acaacagactttattgataaaacttggcctaggccaattgatgtttctaaggctgacggtattatataccctcaaggccgtacatattctaacataactatcacttatcaaggtct |
| | | ttttccctatcagggagaccatggtgatatgtatgtttactctgcaggacatgctacaggcacaactccacaaaagttgtttgtagctaactattctcaggacgtcaaacagtttg |
| | | ctaatgggtttgtcgtccgtataggagcagctgccaattccactggcactgttattattagcccatctaccagcgctactatacgaaaaatttaccctgatttatgctgggttctt |
| | | cagttggtaatttctcagatggtaaaatgggccgcttcttcaatcatactctagttcttttgcccgatggatgtggcactttacttagagctttttattgtattctagagcctcgctct |
| | | ggaaatcattgtcctgctggcaattcctatacttatttgccacttatcacactcctgcaacagattgttctgatggcaattacaatcgtaatgccagtctgaactcttttaaggagt |
| | | attttaatttacgtaactgcacctttatgtacacttataacattaccgaagatgagattttagagtggtttggcattacacaaactgctcaaggtgttcacctcttctcatctcggtat |
| | | gttgatttgtacggcggcaatatgtttcaatttgccaccttgcctgtttatgatactattaagtattattctatcattcctcacagtattcgttctatccaaagtgatagaaaagcttgg |
| | | gctgccttctacgtatataaacttcaaccgttaactttcctgttggatttttctgttgatggttatatacgcagagctatagactgtggttttaatgatttgtcacaactccactgctca |
| | | tatgaatccttcgatgttgaatctggagtttattcagtttcgtctttcgaagcaaaaccttctggctcagttgtggaacaggctgaaggtgttgaatgtgatttttcacctcttctgtc |
| | | tggcacacctcctcaggtttataatttcaagcgtttggtttttaccaattgcaattataatcttaccaaattgattcacttttttctgtgaatgattttacttgtagtcaaatatctccagc |
| | | agcaattgctagcaactgttattcttcactgattttggattacttttcatacccacttagtatgaaatccgatctcagtgttagttctgctggtccaatatcccagtttaattataaaca |
| | | gtccttttctaatcccacatgtttgattttagcgactgttcctcataaccttactactattactaagcctcttaagtacagctatattaacaagtgctctcgtcttattctgatgatcgta |
| | | ctgaagtacctcagttagtgaacgctaatcaatactcaccagtgtatccattgtcccatccactgtgtgggaagacggtgattattataggaaacaactatctccacttgaagg |
| | | tggtggctggcttgttgctagtggctcaactgttgccatgactgagcaattacagatgggctttggtattacagttcaatatggtacagacaccaatagtgtttgccccaagctt |
| | | gaatttgctaatgacacaaaaattgcctctcaattaggcaattgcgtggaatattccctctatggtgtttcgggccgtggtgtttttcagaattgcacagctgtaggtgttcgaca |
| | | gcagcgctttgtttatgatgcgtaccagaatttagttggctattattctgatgatggcaactactactgtttgcgtgcttgtgttagtgttcctgtttctgtcatctatgataaagaaac |
| | | taaaacccacgctactctatttggtagtgttgcatgtgaacacatttcttctaccatgtctcaatactcccgttctacgcgatcaatgcttaaacggcgagattctacatatggccc |
| | | ccttcagacacctgttggttgtgtcctaggacttgttaattcctctttgttcgtagaggactgcaagttgcctcttggtcaatctctctgtgctcttcctgacacacctagtactctca |
| | | cacctcgcagtgtgcgctctgttccaggtgaaatgcgcttggcatccattgatttaatcatcctattcaggttgatcaacttaatagtagttattttaaattaagtatacccactaat |
| | | ttttcctttggtgtgactcaggagtacattcagacaaccattcagaaagttactgttgattgtaaacagtacgtttgcaatggtttccagaagtgtgagcaattactgcgcgagta |
| | | tggccagttttgttccaaaataaaccaggctctccatggtgccaatttacgccaggatgattctgtacgtaatttgtttgcgagcgtgaaaagctctcaatcatctcctatcatac |
| | | caggttttggaggtgactttaatttgacacttctagaacctgtttctatatctactggcagtcgtagtgcacgtagtgctattgaggatttgctatttgacaaagtcactatagctga |
| | | tcctggttatatgcaaggttacgatgattgcatgcagcaaggtccagcatcagctcgtgatcttatttgtgctcaatatgtggctggttacaaagtattacctcctcttatggatgt |
| | | taatatggaagccgcgtatacttcatctttgcttggcagcatagcaggtgttggctggactgctggcttatcctcctttgctgctattccatttgcacagagtatcttttataggtta |
| | | aacggtgttggcattactcaacaggttattcagagaaccaaaagcttattgccaataagtttaatcaggctctgggagctatgcaaacaggcttcactacaactaatgaagct |
| | | tttcagaaggttcaggatgctgtgaacaacaatgcacaggctctatccaaattagctagcgagctatctaatacttttggtgctatttccgcctctattggagacatcatacaac |
| | | gtcttgatgttctcgaacaggacgcccaaatagacagacttattaatggccgtttgacaacactaaatgatttgttgcacagcagcttgttcgttccgaatcagctgactttcc |
| | | gctcaattggctaaagataaagtcaatgagtgtgtcaaggcacaatccaagcgttctggattttgcggtcaaggcacacatatagtgtcctttgttgtaaatgcccctaatggc |
| | | ctttacttcatgcatgttggttattaccctagcaaccacattgaggttgtttctgcttatggtattgcgatgcagctaaccctactaattgtatagcccctgttaatggctactttatt |
| | | aaaactaataacactaggattgttgatgagtggtcatatactggctcgtccttctatgcacctgagcccattacctcccttaatactaagtatgttgcaccacaggtgacatacc |
| | | aaaacatttctactaacctccctcctcctcttctcggcaattccaccgggattgacttccaagatgagttggatgagtttttcaaaaatgttagcaccagtatacctaattttggttc |
| | | cctaacacagattaatactacattactcgatcttacctacgagatgttgtacttcaacaagttgttaaagccdtaatgagtcttacatagaccttaaagagcttggcaattatact |
| | | tattacaacaaatggccgtggtacatttggcttggtttcattgctgggcttgttgccttagctctatgcgtcttcttcatactgtgctgcactggttgtggcacaaactgtatggga |
| | | aaacttaagtgtaatcgttgttgtgatagatacgaggaatacgacctcgagccgcataaggttcatgttcactaattaacgaactattaatgagagttcaaagaccacccactc |
| | | tcttgttagtgttttcactctctatttggtcactgcatcctcaaaacctctctatgtacctgagcattgtcagaattattctggttgcatgcttagggcttgtattaaaactgcccaag |
| | | ctgatacagaggtattatacaaattttcgaattgacgtcccatctgcagaatcaactggtactcaatcagtttctgtcgatcttgagtcaacttcaactcatgatggtcctaccg |
| | | aacatgttactagtgtgaatattttgacgttggttactcagttaattaacgaactctatggattacgtgtactgcttaatcaaatttggcagaagtaccttaactcaccgtatacta |
| | | cttgtttgtacatccctaaacccacagctaagtatacacctttagttggcacttcattgcaccctgtgctgtggaactgtcagctatcctttgctggttatactgaatctgctgttaat |
| | | tctacaaaagattggccaaacaggacgcagctcagcgaatcgcttggttgctacataaggatggaggaatccctgatggatgttccctctacctccggcactcaagtttatt |
| | | cgcgcaaagcgaggaagaggagccattaccaactaagaaactgcgctacgttaagcgtagattttctcttctgcgccatgaagaccttagtgttattgtccaaccaacaca |
| | | ctatgtcagggttacattttcagaccccaacatgtggtatctacgttcgggtcatcatttacactcagttcacaattggcttaaaccttatggcggccaacctgtttctgagtacca |
| | | tattactctagattgctaaatctcactgatgaagatttagctagagatttttcacccattgcgctattttgcgcaatgtcagatttgagctacatgagttcgccttgctgcgcaaa |
| | | actcttgttcttaatgcatcagagatctactgtgctaacatacatagatttaagcctgtgtatagagttaacacggcaatccctactattaaggattggcttctcgttcagggatttt |
| | | ccdttaccatagtggcctccattacatatgtcaatctctaaattgcatgcactggatgatgttactcgcaattacatcattacaatgccatgctttagaacttaccdcaacaaat |
| | | gtttgttactcctttggccgtagatgttgtctccatacggtcttccaatcagggtaataaacaaattgttcattcttatcccattttacatcatccaggattttaacgaactatggcttt |
| | | ctcggcgtctttatttaaacccgtccagctagtcccagtttctcctgcatttcatcgcattgagtctactgactctattgttttcacatacattcctgctagcggctatgtagctgatt |
| | | agctgtcaatgtgtgtctcattcccctattattactgctacgtcaagatacttgtcgtcgcagcattatcagaactatggttctctatttccttgttctgtataactttttattagccattg |
| | | tactagtcaatggtgtacattatccaactggaagttgcctgatagccttcttagttatcctcataatactttggtttgtagatagaattcgtttctgtctcatgctgaattcctacattc |
| | | cactgtttgacatgcgttcccactttattcgtgttagtacagtttcttctcatggtatggtccctgtaatacacaccaaaccattatttattagaaacttcgatcagcgttgcagctgt |
| | | tctcgttgtttttatttgcactcttccacttatatagagtgcacttatattagccgttttagtaagattagcctagtttctgtaactgacttctccttaaacggcaatgtttccactgttttc |
| | | gtgcctgcaacgcgcgattcagttcctcttcacataatcgccccgagctcgcttatcgtttaagcagctctgcgctactatgggtcccgtgtagaggctaatccattagtctctc |
| | | tttggacatatggaaaacgaactatgttaccdttgtccaagaacgaatagggttgttcatagtaaactttttcatttttaccgtagtatgtgctataacactcttggtgtgtatggct |
| | | ttccttacggctactagattatgtgtgcaatgtatgacaggcttcaataccctgttagttcagcccgcattatacttgtataatactggacgttcagtctatgtaaaattccaggata |
| | | gtaaaccccctctaccacctgacgagtgggtttaacgaactccttcataatgtctaatatgacgcaactcactgaggcgcagattattgccattattaaagactggaactttgc |
| | | atggtccctgatctttctcttaattactatcgtactacagtatggatacccatcccgtagtatgactgtctatgtctttaaaatgtttgttttatggctcctatggccatcttccatggc |
| | | gctatcaatatttagcgccgtttatccaattgatctagatcccagataatctctggcattgtagcagctgtttcagctatgatgtggatttcctactttgtgcagagtatccggctg |
| | | tttatgagaactggatcatggtggtcattcaatcctgagactaattgccttttgaacgttccatttggtggtacaactgtcgtacgtccactcgtagaggactctaccagtgtaac |
| | | tgctgttgtaaccaatggccacctcaaaatggctggcatgcatttcggtgcttgtgactacgacagacttcctaatgaagtcaccgtggccaaacccaatgtgctgattgcttt |
| | | aaaaatggtgaagcggcaaagctacggaactaattccggcgttgccatttaccatagatataaggcaggtaattacaggagtccgcctattacggcggatattgaacttgca |
| | | ttgcttcgagcttaggctctttagtaagagtatcttaattgattttaacgaatctcaatttcattgttatggcatcccctgctgcacctcgtgctgtttcctttgccgataacaatgata |
| | | taacaaatacaaacctatctcgaggtagaggacgtaatccaaaaccacgagctgcaccaaataacactgtctcttggtacactgggcttacccaacacgggaaagtccctc |
| | | ttacctttccacctgggcagggtgtacctcttaatgccaattctacccctgcgcaaaatgctgggtattggcggagacaggacagaaaaattaataccgggaatggaattaa |
| | | gcaactggctcccaggtggtacttctactacactggaactggacccgaagcagcactcccattccgggctgttaaggatggcatcgtttgggtccatgaagatggcgccac |
| | | tgatgctccttcaacttttgggacgcggaaccctaacaatgattcagctattgttacacaattcgcgcccggtactaagcttcctaaaaacttccacattgaggggactggagg |
| | | caatagtcaatcatcttcaagagcctctagcttaagcagaaactcttccagatctagttcacaaggttcaagatcaggaaactctacccgcggcacttctccaggtccatctg |
| | | gaatcggagcagtaggaggtgatctactttaccttgatcttctgaacagactacaagcccttgagtctggcaaagtaaagcaatcgcagccaaaagtaatcactaagaaag |
| | | atgctgctgctgctaaaaataagatgcgccacaagcgcacttccaccaaaagtttcaacatggtgcaagcttttggtcttcgcggaccaggagacctccagggaaactttg |
| | | gtgatcttcaattgaataaactcggcactgaggacccacgttggccccaaattgctgagcttgctcctacagccagtgcttttatgggtatgtcgcaatttaaacttacccatca |
| | | gaacaatgatgatcatggcaaccctgtgtacttccttcggtacagtggagccattaaacttgacccaaagaatcccaactacaataagtggttggagcttcttgagcaaaata |
| | | ttgatgcctacaaaaccttccctaagaaggaaaagaaacaaaaggcaccaaaagaagaatcaacagaccaaatgtctgaacctccaaaggagcagcgtgtgcaaggta |
| | | gcatcactcagcgcactcgcacccgtccaagtgttcagcctggtccaatgattgatgttaacactgattagtgtcactcaaagtaacaagatcgcggcaatcgtttgtgtttgg |
| | | caaccccatctcaccatcgcttgtccactcttgcacagaatggaatcatgttgtaattacagtgcaataaggtaattataacccatttaattgatagctatgctttattaaagtgtgt |
| | | agctgtagagagaatgttaaagactgtcacctctgcttgattgcaagtgaacagtgccccccgggaagagctctacagtgtgaaatgtaaataaaaaatagctattattcaatt |
| | | agattaggctaattagatgatttgcaaaaaaaaaaaa |
| 2410 | hCoV- | attgtgagcgatttgcgtgcgtgcatcccgcttcactgatctcttgttagatattttgtaatctaaactttataaaaacatccactccctgtaatctatgcttgtgggcgtagattttt |
| | OC43 | catagtggtgtttatattcatttctgctgttaacagattcagccagggacgtgttgtatcctaggcagtggcccgcccataggtcacaatgtcgaagatcaacaaatacggtct |
| | genome | cgaactacactgggctccagaatttccatggatgtttgaggacgcagaggagaagttggataaccctagtagttcagaggtggatatgatttgctccaccactgcgcaaaa |
| | (Genbank | gctggaaacagacggaatttgtcctgaaaatcatgtgatggtggattgtcgccgacttcttaaacaagagtgttgtgtgcagtctagcctaatacgtgaaattgttatgaatgc |
| | Accession | aagtccatatgatttggaggtgctacttcaagatgattgcagtcccgtgaagcagttttggttacaacccccttaggtatgtctttagaggcatgctatgtgagaggttgtaatc |
| | No. | ctaaaggatggaccatgggtttgtttcggcgtagaagtgtgtgtaacactggtcgttgcactgttaataagcatgtggcctatcagttatatatgattgatcctgcaggtgtctgt |
| | NC_ | cttggtgcaggtcaattcgtgggttgggtcatacccttagcctttatgcctgtgcaatcccggaaatttattgttccatgggttatgtacttgcgtaagcgtggcgaaaagggtg |
| | 006213.1) | cttacaataaagatcatggacgtggcggttttggacatgtttatgattttaaagttgaagatgcttatgaccaggtgcatgatgagcctaagggtaagttttctaagaaggcttat |
| | | gctttaattagagggtatcgtggtgttaaaccacttctctatgtagaccagtatggttgtgattatactggtagtcttgcagatggcttagaggcttatgctgataagacattgcaa |
| | | gaaatgaaggcattatttcctacttggagtcaggaactcctttttgatgtaattgtggcatggcatgttgtgcgtgatccacgttatgttatgagattgcagagtgctgctactata |
| | | cgtagtgttgcatatgttgctaatcctactgaagacttgtgtgatggttctgttgttataaaagaacctgtgcatgtttatgcagatgactctattattttacgtcaatataatttagttg |
| | | acattatgagtcatttttatatggaggcagatacagttgtaaatgctttttatggtgttgctttgaaagattgcggttttgttatgcagtttggttacattgattgcgaacaagactcgt |
| | | gtgattttaaaggttggattcctggtaacatgatagatggttttgcttgcaccacttgtggtcatgtttatgaagtaggtgatttgatggcacaatcttcaggtgttttgcctgttaac |
| | | cctgtattgcatactaagagtgcagcaggctatggtggttttggttgtaaagattcttttactctgtatggccaaactgtagtttattttggaggttgtgtgtattggagtccagcac |
| | | gtaatatatggattcctatattaaaatcctagttaagtcatatgacagtttggtttatactggagttttaggttgcaaggctattgtaaaggaaacaaatctcatttgcaaagctttg |
| | | taccttgattatgttcaacacaagtgtggcaatttacaccaacgggagttgctaggtgtttcagatgtgtggcataaacaattgctattaaatagaggtgtttataaacctctgtta |
| | | gagaatattgattattttaatatgcggcgcgctaaatttagtttagaaacttttactgtttgtgcagatggctttatgccttttcttttagatgatttagttccacgcgcatattatttggc |
| | | agtaagtggtcaagcattttgtgattatgcagataaactttgccatgccgttgtgtctaagagtaaagagttacttgatgtgtctctggattattaggtgcagctatacattatttg |
| | | aattctaagattgttgatttggctcaacattttagtgattttggaacaagtttcgtttctaaaattgttcatttattaagacttttactactagcactgctcttgcatttgcatgggttttat |
| | | ttcatgttttgcatggtgcttatatagtagtggagagtgatatatattttgttaaaaacattcctcgttatgctagtgctgttgcacaagcatttcagagtgttgctaaagttgtactg |
| | | gactctttaagagttacttttattgatggcctttcttgttttaagattggacgtagaagaatttgtctttcaggcagaaaaatttatgaagttgagcgtggcttgttacattcatccca |
| | | attgccattagatgtttatgatttaaccatgcctagtcaagttcagaaagccaagcaaaaacctatttatttaaaaggttctggttctgatttttcattagcggatagtgtagttgaa |
| | | gttgttacaacttcacttacaccatgtggttattctgaaccacctaaagttgcagctaaaatttgcattgtggataatgtttatatggccaaggctggtgacaaatattaccctgttg |
| | | tggttgatgatcatgttggactcttggatcaagcatggagagttccttgtgctggaaggcgtgttacatttaaggaacagcctacagtaaaggagattataagcatgcctaag |
| | | attattaaggttttttatgagcttgacaacgattttaatactattttaaatactgcgtgtggagtgtttgaagtggatgatactgttgatatggaggaattttatgctgtggtgattgat |
| | | gccatagaagagaaactttaccatgtaaggagcttgaaggtgtaggtgctaaagttagtgcctttttacagaaattagaggataatcccctatttttatttgatgaggctggcg |
| | | aggaagttcttgctcctaaattgtattgtgcctttacagctcctgaagatgatgactttcttgaggaaagtgatgttgaagaagatgatgtagaaggtgaggaaactgatttaac |
| | | tgtcacaagtgctggacagccttgtgttgctagtgaacaggaggagtcttctgaagtcttagaggacactttggatgatggtccaagtgtggagacatctgattcacaagttg |
| | | aagaagatgtagaaatgtcggattttgttgatcttgaatctgtgattcaggattatgaaaatgtttgttttgagttttatactacagagccagaatttgttaaagttttgggtctgtatg |
| | | tgcctaaagcaactcgcaacaattgctggttgcgatcagttttggcagtgatgcagaaattgccctgtcaatttaaagataaaaatttgcaggatctttgggtgttatacaagca |
| | | acagtatagtcagttgtttgttgataccttggttaataagatacctgctaatattgtacttccacaaggtggttatgttgctgattttgcatattggtttttaaccttatgtgattggcag |
| | | tgtgttgcatactggaaatgcattaaatgtgatttagctcttaagcttaaaggcttggatgctatgttcttttatggtgatgttgtttcacatatatgcaagtgtggtgagtctatggta |
| | | cttattgatgttgatgtgccatttacagcccactttgctcttaaagataagttgttttgtgcatttattactaagcgtattgtgtataaagcagcttgtgttgtggatgttaatgatagtc |
| | | attctatggctgttgttgatggtaaacaaattgatgatcatcgtatcactagtattactagtgataagtttgattttattattgggcatggtatgtcattttcaatgactacttttgaaatt |
| | | gcccaattgtatggttcttgtataacacctaatgtgtgttttgttaaaggtgatataattaaagtatctaagcttgttaaagcagaagttgttgtaaaccctgctaatggccatatgg |
| | | cacatggtggtggtgttgcaaaagctattgcagtagcagctggacagcagtttgttaaagagactaccgatatggttaagtctaaaggagtttgtgctactggagattgttatg |
| | | tactacagggggcaaattatgtaaaactgtgcttaatgttgttggacctgatgcgagaacacagggtaaacaaagttatgtattgttagagcgtgtttataaacatcttaacaa |
| | | ctatgactgtgttgttacaactttgatctcagctggtatatttagtgtgccttctgatgtgtctttaacatatctacttggtactgctaagaaacaagttgttcttgttagcaataatcaa |
| | | gaggattttgatcttatttctaagtgtcagataactgctgttgagggcactaagaaattggcagcgcgtctttatttaatgttggacgttccattgtttacgaaacagatgctaata |
| | | agttgattttaatcaatgacgttgcatttgtttcgacatttaatgttttacaggatgttttatccttaagacatgatatagcacttgatgatgatgcacgaaccttcgttcagagcaat |
| | | gttgatgttgtacctgagggttggcgtgttgtcaataagttttatcaaattaatggtgttagaaccgttaagtattttgagtgtactggaggcatagatatatgcagccaggataa |
| | | agtttttggttatgtacagcagggtatttttaataaggctactgttgctcaaattaaagccttgtttttggataaagtggacatcttgctaactgttgatggtgttaatttcactaatag |
| | | gtttgtgcctgttggtgaaagttttggtaagagtctaggaaatgtgttttgtgatggagttaatgtcacgaagcataagtgtgatataaattataaaggtaaagtctttttccagttt |
| | | gataatctttctagtgaagatttaaaggctgtaagaagttcctttaattttgatcagaaggaattgcttgcctattacaacatgcttgttaattgttttaagtggcaggttgttgttaat |
| | | ggtaagtatttcacttttaagcaagctaataacaattgttttgttaatgtttcttgcttaatgctccagagtttgcatctgacatttaaaattgttcaatggcaagaggcatggcttga |
| | | atttcgttctggccgccctgctagatttgtagctttggttttggccaaaggtgggtttaaatttggagatcctgctgattctagagatttcttgcgtgttgtgtttagtcaagttgattt |
| | | gactggggcaatatgtgattttgaaattgcatgtaaatgtggtgtaaagcaggaacagcgtactggtaggacgctgttatgcattttggtacattgagtcgtgaagatcttga |
| | | gattggttataccgtggactgttcttgcggtaaaaagctaattcattgtgtacgatttgatgtaccatttttaatttgcagtaatacacctgctagtgtaaaattacctaagggtgta |
| | | ggaagtgcaaatatttttataggtgataaggttggtcattatgttcatgttaagtgtgaacaatcttatcagctttatgatgcttctaatgttaagaaggttacagatgttactggca |
| | | agttgtcagattgtctgtatcttaaaaatttgaaacaaacttttaaatcggtgttaaccacctattatttggatgatgttaagaaaattgagtataaacctgacttgtcacaatattatt |
| | | gtgacggaggtaagtattatactcagcgtattattaaagcccaatttaaaacattcgagaaagtagatggtgtgtatactaattttaaattgataggacacaccgtagtgacag |
| | | tcttaatgctaagttgggttttgatagctctaaagagtttgttgaatataagattactgagtggccaacagctacaggtgatgtggtgttggctactgatgatttgtatgttaagag |
| | | atatgagaggggttgtattacttttggtaaacctgttatatggttaagccatgagaaagcttccctcaattattaacatattttaatagaccttcattggttgatgataataaatttga |
| | | tgttttaaaagtggatgatgttgacgatggtggtgacagctcagagagtggtgccaaagaaaccaaagaaatcaacattattaagttaagtggtgttaaaaaaccatttaagg |
| | | ttgaagatagtgtcattgttaatgatgatactagtgaaaccaaatatgttaagagtttgtctattgttgatgtgtatgatatgtggcttacaggttgtaagtatgttgttagaactgct |
| | | aatgattgagcagagcagttaacgtacctacaatacgtaagtttataaaatttggtatgactcttgttagtataccaattgatttgttaaatttaagagagattaagcctgctgtta |
| | | atgtggttaaagagtgcgaaataaaatttctgtatgattaatt-ttattaaatggctttttgtcttattatttggctggattaaaatatccgctgataataaagtaatctacaccacag |
| | | aaattgcatcaaagcttacgtgtaagcttgtagctttagcttttaaaaatgcatttttgacatttaagtggagtatggttgctagaggtgcttgcattatagcgactatatttctattgt |
| | | ggtttaattttatatatgccaatgtaatttttagtgatttttatttgcctaaaatcggtttcttgccgacttttgttggtaagattgcacagtggattaagaacacttttagtcttgtaacta |
| | | tttgtgatctatattccattcaggatgtgggttttaagaatcagtattgtaatggaagtattgcatgtcagttctgcttggcaggatttgatatgttagataattataaagccattgat |
| | | gtagtacagtatgaagctgataggagagcatttgttgattatacaggtgtgttaaagattgtcattgaattgatagttagttacgccctgtatacggcatggttttatccattgtttg |
| | | cccttatcagtattcagatcttgaccacttggctgcctgagctttttatgcttagtacattacattggagttttaggttgctggtggctttagctaatatgttaccagcacatgtgttta |
| | | tgaggttttatattattattgcctcttttattaagctctttagcttgtttaggcatgttgcctatggttgtagtaaatctggttgtttgttttgttacaagaggaatcgtagtctacgtgtta |
| | | aatgtagtactatcgttggtggcatgatacgctattacgatgttatggctaatggtggcactggcttttgttcaaaacatcaatggaattgcattgattgtgattcttataaaccag |
| | | gtaatacttttattactgttgaggccgctcttgatctatctaaggaattgaaacggcccattcagcctacagatgttgcttatcatacggttactgatgttaagcaagttggttgttc |
| | | tatgcgcttgttctatgatcgtgatggacagcgcacatatgatgatgttaatgctagtttgtttgtggattatagtaatttgctacattctaaggttaagagtgtgcctaatatgcatg |
| | | ttgtggtagtggaaaatgatgctgataaagccaattttctgaatgctgctgtattttatgcacagtctttgtttagacctattttaatggttgataaaaatctgataactactgctaac |
| | | actggtacgtctgttacagaaactatgtttgatgtttatgtggatacatttttgtctatgtttgatgtggataaaaagagtcttaatgattaatagcaactgcgcattatctataaaa |
| | | cagggtacgcagatttataaagttttggatacctttttaagctgtgctcgtaaaagttgttctattgattcagatgttgatactaagtgtttagctgattctgtcatgtctgctgtatcg |
| | | gcaggtcttgaattgacggatgaaagttgtaataacttggtgccaacatatttgaagagtgacaacattgtggcagctgatttaggtgttctgattcaaaattctgcaaagcatg |
| | | tgcagggtaatgttgctaaaatagctggtgtttcctgtatatggtctgtggatgcttttaatcagtttagttctgatttccagcataaattgaagaaagcatgttgtaaaactggtttg |
| | | aaactgaagcttacttataataagcagatggctaatgtctctgttttaactacaccattagtcttaaagggggtgcagtttttagttattttgtttatgtgtgttttgtgttgagtttggt |
| | | ctgttttattggactgtggtgcttaatgcccacttacacagtacacaaatcagattttcagcttcccgtttatgccagttataaagttttagataatggtgttattagagatgttagcg |
| | | ttgaagatgtttgtttcgctaacaaatttgaacaatttgatcaatggtatgagtctacatttggtctaagttattatagtaacagtatggcttgtcccattgttgttgctgtaatagatc |
| | | aggattttggctctacagtgtttaatgtccctaccaaagtgttacgatatggttatcatgtgttgcactttattacacatgcactttctgctgatggagtgcagtgttatacgccaca |
| | | tagtcaaatatcgtattctaatttttatgctagtggctgtgtgattcctctgcttgcactatgtttacaatggccgatggtagtccacaaccttattgttatacagaggggcttatgc |
| | | aaaatgcttactgtatagttcattggtacctcacgtgcggtataatcttgctaatgctaaaggttttatccgttttccagaagtgttgcgagaagggcttgtacgtatcgtgcgta |
| | | ctcgttctatgtcgtattgcagagttggattatgtgaggaagctgatgagggtatatgattaattttaatggttcttgggtgcttaataatgattattatagatcattgcctgggacc |
| | | ttttgtggtagagatgtttttgatttaatttatcagctatttaaaggtttagcacagcctgtggattttttggcattgactgctagttccattgctggtgctatactcgctgtaattgttgt |
| | | tttggtgttttattacctaataaagcttaaacgtgatttggtgattacaccagtgttgtttttgttaacgtgattgtgtggtgtgtaaattttatgatgctttttgtgtttcaagtttacccc |
| | | atactttcttgtgtatatgctatttgttatttttatgccacgattatttcccttcggagataagtgtgataatgcacttacaatggctagttatgtatggcactattatgcctttatggtttt |
| | | gtttgctatatatagctgttgttgtttcaaatcatgctttttgggtattttcttactgcagaaagcttggtacttctgttcgtagtgatggtacatttgaagaaatggctctcactactttt |
| | | atgattacaaaagattcttattgtaagcttaagaattctttgtctgatgttgatttaatagatatttgagtttgtataataaatataggtattacagcggtaaaatggatactgctgcat |
| | | atagggaggctgcttgctctcagttggctaaagcaatggacacatttaccaataataatggtagtgatgtgctttaccaaccgcctactgcttccgtctcaacttcattcttgcaa |
| | | tctggtattgtgaaaatggtaaatcctacttctaaggtagaaccatgtgttgtcagtgttacctatggtaatatgacattgaatggtttatggttggatgacaaggtctactgtccc |
| | | agacatgtaatatgttctgcttcagatatgactaatccagattatacaaatttgttgtgtagagtaacatcaagtgattttactgtattgtttgatcgtctaagccttacagtgatgtct |
| | | tatcaaatgcggggttgtatgcttgttcttacagtgaccdgcaaaattctcgtacgccaaaatatacatttggtgtggttaaacctggtgagacttttactgttttagctgcttata |
| | | acggcaaaccacaaggagcctttcatgtaactatgcgtagtagttataccattaagggttcctttttatgcggatcttgtggatctgttggttatgtaataatgggtgattgtgtta |
| | | aatttgtttatatgcatcaattggagcttagtactggttgtcatactggtactgacttcaatggggatttttatggtccttataaggatgctcaggttgttcagttgctcattcaggatt |
| | | atatacaatctgttaattttgtagcatggctttatgctgctatacttaacaattgtaattggtttgtacaaagtgataagtgttctgtagaagattttaatgtgtgggctctgtccaatg |
| | | gatttagccaagttaaatctgaccttgttatagatgctttagcttctatgactggtgtgtctttggaaacactgttggctgctattaagcgtcttaagaatggtttccaaggacgtca |
| | | gattatgggtagttgctcttttgaggatgaattgacacctagcgatgtttatcaacaactcgctggtatcaagttacaatcaaaacgcactagattgtttaaaggcactgtttgttg |
| | | gattatggcttctacatttttgtttagttgcataattacagcatttgtgaaatggactatgtttatgtatgtaactactaatatgtttagtattacgttttgtgcactttgtgttataagtttg |
| | | gccatgttgttggttaagcataagcatctttatttgactatgtatataactcctgtgctttttacactgttgtataacaactatttggttgtgtacaagcatacatttagaggctatgtct |
| | | atgcatggctatcatattatgttccatcagttgagtacacttatactgatgaagttatttatggcatgttattgcttgtaggaatggtattgttacattacgtagcattaaccatgattt |
| | | gttttcttttataatgtttgttggtcgtttgatttctgttttctctttgtggtacaagggttctaacttagaggaagaaattcttcttatgttggcttccctttttggtacttacacatggaca |
| | | acagttttatctatggctgtagcaaaggttattgctaagtgggttgctgtgaatgtcttgtatttcacagatatacctcaaattaagatagtgatttgtgctatttgtttattggttatat |
| | | tattagctgttattggggcttgttttccttgatgaacagtttgtttagaatgcctttgggtgtttataattataaaatttcagtacaggaattaagatatatgaatgctaatggattgcg |
| | | ccctcctaagaatagttttgaagcccttatgcttaattttaagagttgggtattggaggtgttccaatcattgaagtatctcaatttcaatcaaaattgactgatgtcaaatgtgcta |
| | | atgtcgtcttgcttaattgcttgcaacatttgcatgttgcttctaattctaagttgtggcattattgtagcactttgcacaatgaaatacttgccacttcggatctgagtgttgatttga |
| | | aaagcttgctcagttattaattgttttgtttgctaatccagctgctgtggatagcaagtgcctgactagtattgaagaagtttgcgatgattacgcaaaggacaatactgttttgca |
| | | ggctttacagagtgaatttgttaatatggctagcttcgttgaatatgaagttgctaagaaaaatcttgatgaggcgcgttttagtggttctgctaatcaacagcagttaaaacagc |
| | | tagagaaagcctgtaatattgctaaatctgcttatgaacgcgaccgtgctgtagcaaaaaagttggagcgtatggctgatttggctctcactaatatgtataaagaagctagaa |
| | | ttaatgataagaagagtaaggttgtttctgccttgcaaactatgctttttagtatggtgcgtaagttagataatcaagctctgaattcaatattagataacgctgtgaagggttgtgt |
| | | accattgaatgcaataccttcattggcagcaaatactctgaatataattgtaccagataaaagtgtttatgaccaggtagttgataatgtctatgttacctatgcgggtaatgtatg |
| | | gcagattcaaactatccaggattcagatggtacaaataagcagttgaatgagatatctgatgattgtaactggccactagttattattgcaaatcggtataatgaggtatctgct |
| | | actgttttgcaaaataatgaattaatgcctgctaagttgaaaattcaggttgttaatagtggtccagatcagacttgtaatacacctactcaatgttactataataatagtaacaatg |
| | | ggaagattgtttatgctatacttagtgatgttgatggtcttaagtatacaaaaattcttaaagatgatggcaattttgttgttttggagttagatcctccttgtaaatttactgttcaaga |
| | | tgctaaaggtcttaaaattaagtacctttattttgtaaaaggttgtaacacactagcaagaggctgggttgttggtacaatttcttctacagttagattgcaagctggaactgctac |
| | | tgaatatgcttccaactcatctatattgtattatgtgcgttttctgtagatcctaagaaaacgtatttagattttatacaacaaggaggaacacctattgccaattgtgttaaaatgtt |
| | | gtgtgaccatgctggtaccggtatggccattactgttaaacccgatgctaccactagtcaggattcatatggtggtgcgtctgtttgtatatattgccgcgcacgagttgaaca |
| | | cccagatgttgatgggttgtgcaaattacgcggcaagtttgtacaagtgcctgtaggtataaaagatcctgtgtcttatgttttgacacatgatgtttgtcgagtttgtggattttg |
| | | gcgggatggaagttgttcatgtgttagcactgacactactgttcaatcaaaagatactaattttttaaacgggttcggggtacgagtgtagatgcccgtctcgtaccctgcgcc |
| | | agtggtttatctactgatgtacaattaagggcatttgatatttacaatgctagtgttgctggcattggtttacatttaaaagttaattgttgccgttttcagcgtgttgatgagaacgg |
| | | tgataaattagatcagttattgttgttaagaggacagatctgactatatataatagagagatgaaatgctatgagcgtgtaaaagattgtaagtttgtggctgaacacgatttctt |
| | | tacatttgatgtagaaggtagtcgtgtgccacacattgtacgcaaggatttaacaaagtatactatgttggatctttgctatgcattgcgacattttgatcgcaatgattgcatgct |
| | | gctttgtgacattctctctatatatgctggttgtgaacaatcctactttactaagaaggattggtatgattttgttgaaaatcctgatattattaatgtgtataaaaagctaggacctat |
| | | ttttaatagagccctagttagcgctactgagtttgcggacaaattggtggaggtaggcttagtaggcgttttaacacttgataatcaagatttaaatggtaaatggtatgattttg |
| | | gtgactatgttattgcagccccaggatgtggtgttgctatagcagattcttattattcttatatcatgcctatgctgaccatgtgtcatgcattggattgcgaattgtatgtgaataat |
| | | gcttatagactatttgatcttgtacagtatgattttactgattacaagcttgaattgtttaataagtattttaagcactggagtatgccatatcatcctaacactgttgattgtcaggat |
| | | gatcggtgtattatacattgtgctaattttaacatactttttagtatggttttacctaatacatgttttgggcctcttgttaggcaaatttttgtggatggtgtgccttttgttgtttcaattg |
| | | gctaccattataaagaacttggtattgtgatgaatatggatgtggatacacatcgttatcgcttgtctttaaaagacttgatttatatgctgctgatccagctttgcatgtagcttct |
| | | gctagtgcattgtatgatttacgcacttgctgttttagtgttgccgctataacaagcggtgtaaaatttcaaacagttaaacctggtaattttaatcaggatttttatgattttgttttaa |
| | | gtaaaggcctgcttaaagagggtagctcagttgatctgaagcactttttattacacaggatggtaatgctgctattactgattataattattataagtataatttgcccaccatggt |
| | | ggacattaagcagttgttgtttgttttggaagttgtttataagtattttgagatttatgatggtgggtgtataccggcatcacaagtcattgttaataattatgataagagtgctggct |
| | | atccatttaacaaatttggaaaagccaggctctattatgaagcattatcatttgaggaacaggatgaaatttacgcttatactaagcgtaatgtcctgccaacacttactcaaatg |
| | | aatttgaaatatgctattagtgctaagaatagagcccgcactgttgctggtgtttccatacttagtactatgactggcagaatgtttcatcaaaaatgtttgaaaagtatagcagct |
| | | acacgtggtgttcctgtagttataggcaccactaaattttatggtggctgggatgatatgttacgccgccttattaaagatgttgacaatcctgtacttatgggttgggattatcct |
| | | aagtgtgatcgtgctatgccaaacctactacgtattgttagtagtttggtattagcccgaaaacatgagacatgttgttcgcaaagcgataggttttatcgacttgcgaatgaat |
| | | gcgcacaagttttgagtgaaattgttatgtgtggtggctgttattatgttaagcctggtggcactagtagtggtgatgcaactactgcttttgctaattcagtctttaacatatgtca |
| | | agagtttcagccaatgtatgtgccttaatgtcatgcaatggcaataagattgaagatcttagtatacgtgctcttcagaagcgcttatactcacatgtgtatagaagtgataagg |
| | | ttgattcaacctttgtcacagaatattatgaatttttaaataagcattttagtatgatgattttgagtgatgatggggttgtgtgttataattctgattatgcgtccaaagggtatattgc |
| | | taatataagtgcctttcaacaggtattatattatcaaaataacgtttttatgtcagaatccaaatgttgggttgaacatgacataaataatggacctcatgaattctgttcacaacac |
| | | acaatgcttgtaaagatggatggtgacgatgtctaccttccatatcctaatcctagtcgtatattaggagaggatgttttgtagatgatttgttaaagactgatagtgttcttttaat |
| | | agaacgatttgtaagtcttgcaatagatgcttatccacttgtgtatcatgaaaatgaagaataccaaaaggtttttcgtgtttatttggcgtatataaagaagttgtacaatgacct |
| | | gggtaatcagatcttggatagctacagtgttattttaagtacttgtgatggacaaaagttcactgatgagtccttttacaagaacatgtatttaagaagtgcagttatgcagagtg |
| | | ttggagcttgcgtggtctgctcttctcaaacatcattacgttgtggcagttgcatcagaaagcctcttctttgctgcaagtgttgttatgatcatgttatggcgactgatcataaata |
| | | tgtcttgagtgtttcaccatatgtgtgtaatgcaccaggatgtgatgtaaatgatgttaccaaattgtatctaggtggtatgtcatattattgtgaagaccataagccacaatattca |
| | | ttcaagttggtaatgaatggtaggtttttggtctatataaacaatcttgtacaggatctccgtacatagacgattttaatcgtatagctagttgtaaatggaccgatgtggatgatt |
| | | acatactagctaatgaatgtacagagcgcttgaaattgtttgctgcagaaacgcaaaaggcaaccgaggaagcctttaagcagagttatgcatcagcaacaatacaagaga |
| | | ttgttagtgagcgcgaattgattctctcttgggagattggaaaagttaagccaccacttaataaaaattatgtttttactggctaccattttactaaaaatggtaagacagttttagg |
| | | tgagtatgtttttgataagagtgagttgactaatggtgtgtattatcgcgccacaaccacttataagctatctgtaggagatgtttttgttttaacctctcattcagtagctaatttaa |
| | | gtgctcctacgcttgttccgcaggagaattatagtagtattagatttgctagtgtttatagtgtgcttgagacgtttcagaacaatgttgttaattatcaacacattggtatgaaacg |
| | | ttactgcaccgtgcaaggacctcctggtacagggaagtcacatcttgctattggtcttgctgtattctattgtacagcacgtgttgtatacacagcggccagccatgcagctgtt |
| | | gacgcattgtgtgaaaaagcatataaatttttgaatataaatgattgcactcgtattgttccggccaaggtcagggtggagtgctatgataagtttaaaattaatgacaccactc |
| | | gtaagtatgtgtttactaccataaatgcattacctgagatggtgactgatattgttgttgtagatgaagttagtatgcttaccaattatgagctttctgttattaatgctcgtattcgcg |
| | | ctaagcattatgtttatattggtgatcctgctcaattgccagcaccacgtgtgttattgagcaagggtacacttgaacctaaatattttaacactgttactaagctcatgtgttgctt |
| | | agggccagacatttttcttggtacatgttatagatgtcctaaggaaatcgttgatacagtgtccgccttggtttatgaaaataagcttaaggctaagaatgagagtagttcattgt |
| | | gttttaaggtctattataagggcgttacaacacatgaaagttctagtgctgtaaatatgcagcagatttatttgattaataagtttttgaaggctaaccattgtggcataaagctgt |
| | | ttttattagcccatataatagtcagaactttgcagctaagcgtgttttgggtttacaaacccaaaccgtggattctgctcaaggttctgaatatgattatgttatatattcacagact |
| | | gcagaaacagcgcattctgtaaatgttaatcgcttcaatgttgctattactcgagccaagaaaggtattctttgtgttatgagtaatatgcagttgtttgaagcattacagtttacta |
| | | cattgaccttagataaagtgccacaggccgtcgaaactaaagttcaatgtagtactaatttatttaaagattgtagcaagagttatagcggttatcacccagctcatgctccttca |
| | | tttttggcagtagatgacaaatataaggcaactggcgatttagccgtgtgtcttggtattggtgattctgctgttacatattcaagattaatatcactcatgggttttaaattggatgt |
| | | tacccttgatgggtattgtaagctttttataactaaagaagaagagttaaacgcgtgcgtgcctgggttggctttgatgctgaaggtgctcatgccacgcgtgatagcattgg |
| | | gacaaatttcccacttcaattaggattttccacaggaattgattttgttgtggaagccactggtttgtttgctgatagagatggttacagctttaaaaaggctgtggcgaaagctc |
| | | ctcctggtgaacaatttaagcacctcatccattgatgacgagaggtcatcgctgggatgttgttagacctagaatagtacaaatgtttgcagatcatttaattgatctgtctgatt |
| | | gtgttgtgctagttacatgggcagccaactttgagctcacttgtaccgctactttgcaaaagtagggcgtgagatttcttgtaatgtatgcactaaacgtgccacagtttacaat |
| | | tctagaactggttactatggttgttggcgccatagtgttacatgtgattacttgtataatccacttattgttgatattcaacagtggggatatattggttattatcaagtaatcatgatt |
| | | tatattgtagtgtccataaaggagcacatgttgcttcctctgatgctataatgacacggtgtttggccgtttatgattgatttgcaataatattaattggaatgtggagtatcccatc |
| | | atttcaaatgagttaagtattaatacctcttgtagggtcttgcagcgtgtgattcttaaagctgccatgctctgcaacagatatactttgtgttatgatattggcaacccaaaagcg |
| | | attgcctgtgtcaaagattttgattttaagttctatgatgcccaaccaattgttaagtctgttaagactcttttgtattatttgaggcacataaggactcttttaaagacggtttgtgta |
| | | tgttttggaactgtaatgtggataagtatccaccgaatgcagttgtatgtagatttgacactagagtgttgaataatttaaatcttcctggctgtaatggaggtagtttgtatgttaat |
| | | aaacatgcattccacactaaaccattgctagggcagcctttgagcatttgaagcctatgccattcttctattattcagatacgccttgtgtgtatatggatggcatggatgctaa |
| | | gcaggttgattatgtacctttgaaatctgccacgtgcatcacaagatgcaatttaggtggtgcagtttgtttaaaacatgctgaagagtatcgtgagtacttagagtcttacaata |
| | | cagctactacagcaggttttactttttgggtctataagacatttgatttttataatttgtggaatacgttcaccaagctacaaagcttggagaatgttgtatataatttagtcaagact |
| | | ggtcattatacaggacaggctggtgaaatgccttgtgccattataaatgataaagttgtggctaagatcgataaggaggatgttgtcatttttattaataatacaacataccctac |
| | | taatgtggccgttgaattatttgccaagcgcagtgttcgacaccacccagagcttaagctctttagaaatttaaatatagacgtgtgttggaagcacgtcatttgggattatgct |
| | | agagaaagtatattttgcagtaatacctatggtgtctgcatgtatacagatttaaagttcattgataaattgaatgtcctttttgatggtcgtgataatggtgctcttgaagcttttaa |
| | | acgttctaataatggcgtttacatttccacgacaaaagttaagagtctttcgatgataagaggtccaccgcgtgctgaattaaatggcgtagtggtggacaaggttggagaca |
| | | ctgattgtgtgttttattttgctgtgcgtaaagaaggtcaggatgtcatcttcagccaattcgacagcctgggagtcagctctaaccagagcccacaaggtaatctggggagt |
| | | aatggtaaacccggtaatgtcggtggtaatgatgactgtcaatctctactatctttacacaaagccgtgttattagctcttttacatgtcgtactgatatggaaaaagattttatag |
| | | ctttagatcaagatgtgtttattcagaagtatggtttggaggactatgcctttgaacacattgtttatggtaacttcaaccagaagattattggtggtttgcatttgttaataggcttg |
| | | taccgaagacagcaaacttccaatctggttgttcaggagtttgtttcatatgactccagcatacactcttattttatcactgacgagaagagtggtggtagtaagagtgtttgcac |
| | | tgttatagatattttgttggatgattttgtggctcttgttaagtcacttaatcttaattgtgtgagtaaggttgttaatgttaatgttgattttaaagattttcagtttatgattggtgtaac |
| | | gatgagaaagttatgactttctatcctcgtttgcaagctgcatctgactggaagcctggttattctatgcctgtattatataagtatttgaattctccaatggaaagagttagtctct |
| | | ggaattatgggaagccagttactttgcctacaggctgtatgatgaatgttgctaagtatactcagttatgtcaatatctgaatactacaacattagctgtacctgttaatatgcga |
| | | gttttgcatttaggtgcaggttcagaaaaaggagtagcaccgggttctgcagttcttaggcagtggttgcctgctggtactattcttgtagataacgatttatacccatttgttagt |
| | | gacagtgtcgctacatattttggggattgtataactttaccattgattgtcaatgggatttgataatttctgatatgtatgaccctattactaagaacataggggagtacaatgtga |
| | | gtaaagatggtttctttacatacatttgtcatatgattcgagacaagttagctctgggtggcagtgttgctataaaaataacagagttttcttggaatgcagaattatataagttaat |
| | | ggggtattttgcattttggactgtgttttgcacaaatgcaaatgcttcttctagtgaaggatttttaattggcataaattatttgtgtaagcccaaggttgagatagatggaaatgtta |
| | | tgcatgccaattatttgttttggagaaattccacagtttggaacgggggtgcttatagcctgtttgatatggctaaattcccgcttaagttggctggtactgccgtaataaatttaa |
| | | gagcagaccagattaatgatatggtttattcccttcttgaaaagggtaaactacttattagagatacaaataaagaagt-tttcgttggtgacagtttggttaatgtaatctaaacttt |
| | | aaaaatggctgtcgcttatgcagacaagcctaatcattttatcaattttccacttacccattttcagggttttgtgttaaattataaaggtttacaatttcaaattctcgatgaaggagt |
| | | ggattgtaaaatacaaacagcgccacacattagtcttactatgctggacatacagcctgaagactataaaagtgttgatgtcgctattcaagaagttattgatgatatgcattgg |
| | | ggtgatggttttcagattaaatttgagaatcctcacatcctaggaagatgcatagttttagatgttaaaggtgtagaagaattgcatgacgatttagttaattacattcgtgataaa |
| | | ggttgtgttgctgaccaatccaggaaatggattggccattgcaccatagctcaactcacggatgcagcactgtccattaaggaaaatgttgattttataaacagcatgcaattc |
| | | aattataaaatcaccatcaacccctcatcaccggctagacttgaaatagttaagctcggtgctgaaaagaaagatggtttttatgaaaccatagttagtcactggatgggaatt |
| | | cgttttgaatacacatcacccactgataagctagctatgattatgggttattgttgtttagatgtggtacgtaaagagctagaagaaggcgatcttcccgagaatgatgatgatg |
| | | cttggtttaagctatcgtaccattatgaaaacaattcttggttcttccgacatgtctacaggaaaagttttcatttccgtaaggcttgtcaaaatttagattgtaattgtttggggtttt |
| | | atgaatcttcagttgaagaatattaaactcagtgaaaatgtttttgcttcctagatttattctagttagctgcataattggtagcttaggtttttacaaccctcctaccaatgttgtttcg |
| | | catgtaaatggagattggtttttatttggtgacagtcgttcagattgtaatcatattgttaatatcaacccccataattattcttatatggaccttaatcctgttctgtgtgattctggta |
| | | aaatatcatctaaagctggcaactccatttttaggagttttcactttaccgatttttataattacacaggcgaaggtcaacaaattattttttatgagggtgttaattttacgccttatc |
| | | atgcctttaaatgcaaccgttctggtagtaatgatatttggatgcagaataaaggcttgttttatactcaggtttataagaatatggctgtgtatcgcagccttacttttgttaatgta |
| | | ccatatgtttataatggctccgcacaagctacagctctttgtaaatctggtagtttagtccttaataaccctgcatatatagctcctcaagctaactctggggattattattataagg |
| | | ttgaagctgatttttatttgtcaggttgtgacgagtatatcgtaccactttgtatttttaacggcaagtttttgtcgaatacaaagtattatgatgatagtcaatattattttaataaaga |
| | | cactggtgttatttatggtctcaattctacagaaaccattaccactggttttgatcttaattgttattatttagttttaccctctggtaattatttagccatttcaaatgagctattgttaac |
| | | tgttcctacgaaagcaatctgtcttaataagcgtaaggattttacgcctgtacaggttgttgattcgcggtggaacaatgccaggcagtctgataacatgacggcggttgcttg |
| | | tcaacctccgtactgttattttcgtaattctactaccaactatgttggtgtttatgatattaatcatggagatgctggttttactagcatacttagtggtttgttatataattcaccttgttt |
| | | ttcgcagcaaggcgtttttaggtatgataatgttagcagtgtctggcctctctacccctatggcagatgtcccactgctgctgatattaatatccctgatttacccatttgtgtgtat |
| | | gatccgctaccagttattttgcttggcattcttttgggcgttgcgattgtaattattgtagttttgttgttatattttatggtggataatgttactaggctgcatgatgcttagaccataat |
| | | ctaaacatgtttttgatacttttaatttccttaccaacggcttttgctgttataggagatttaaagtgtacttcagataatattaatgataaagacaccggtcctcctcctataagtact |
| | | gatactgttgatgttactaatggtttgggtacttattatgttttagatcgtgtgtatttaaatactacgttgtttcttaatggttattaccctacttcaggttccacatatcgtaatatggc |
| | | actgaagggaagtgtactattgagcagactatggtttaaaccaccatttattctgattttattaatggtatttttgctaaggtcaaaaataccaaggttattaaagatcgtgtaatgt |
| | | atagtgagttccctgctataactataggtagtacttttgtaaatacatcctatagtgtggtagtacaaccacgtacaatcaattcaacacaggatggtgataataaattacaaggt |
| | | cttttagaggtctctgtttgccagtataatatgtgcgagtacccacaaacgatttgtcatcctaacctgggtaatcatcgcaaagaactatggcatttggatacaggtgttgtttc |
| | | ctgtttatataagcgtaatttcacatatgatgtgaatgctgattatttgtattttcatttttatcaagaaggtggtactttttatgcatattttacagacactggtgttgttactaagtttttg |
| | | tttaatgtttatttaggcatggcgctttcacactattatgtcatgcctctgacttgtaatagtaagcttactttagaatattgggttacacctdcacttctagacaatatttactcgcttt |
| | | caatcaagatggtattatttttaatgctgttgattgtatgagtgattttatgagtgagattaagtgtaaaacacaatctatagcaccacctactggtgtttatgaattaaacggttaca |
| | | ctgttcagccaatcgcagatgtttaccgacgtaaacctaatcttcccaattgcaatatagaagcttggcttaatgataagtcggtgccctctccattaaattgggaacgtaagac |
| | | attttcaaattgtaattttaatatgagcagcctgatgtcttttattcaggcagactcatttacttgtaataatattgatgctgctaagatatatggtatgtgtttttccagcataactatag |
| | | ataagtttgctatacccaatggcaggaaggttgacctacaattgggtaatttgggctatttgcagtcatttaactatagaattgatactactgcaacaagttgtcagttgtattata |
| | | atttacctgctgctaatgtttctgttagcaggtttaatccttctacttggaataagagatttggttttatagaagattctgtttttaagcctcgacctgcaggtgttcttactaatcatga |
| | | tgtagtttatgcacaacactgtttcaaagctcctaaaaatttctgtccgtgtaaattgaatggttcgtgtgtaggtagtggtcctggtaaaaataatggtataggcacttgtcctgc |
| | | aggtactaattatttaacttgtgataatttgtgcactcctgatcctattacatttacaggtacttataagtgcccccaaactaaatctttagttggcataggtgagcactgttcgggt |
| | | cttgctgttaaaagtgattattgtggaggcaattcttgtacttgccgaccacaagcatttttgggttggtctgcagactcttgtttacaaggagacaagtgtaatatttttgctaattt |
| | | tattttgcatgatgttaatagtggtcttacttgttctactgatttacaaaaagctaacacagacataattcttggtgtttgtgttaattatgacctctatggtattttaggccaaggcatt |
| | | tttgttgaggttaatgcgacttattataatagttggcagaaccttttatatgattctaatggtaatctctacggttttagagactacataacaaacagaacttttatgattcgtagttgc |
| | | tatagcggtcgtgtttctgcggcctttcacgctaactcttccgaaccagcattgctatttcggaatattaaatgcaactacgtttttaataatagtcttacacgacagctgcaaccc |
| | | attaactattttgatagttatcttggttgtgttgtcaatgcttataatagtactgctatttctgttcaaacatgtgatctcacagtaggtagtggttactgtgtggattactctaaaaaca |
| | | gacgaagtcgtggagcgattaccactggttatcggtttactaattttgagccatttactgttaattcagtaaacgatagtttagaacctgtaggtggtttgtatgaaattcaaatac |
| | | cttcagagtttactataggtaatatggtggagtttattcaaacaagctctcctaaagttactattgattgtgctgcatttgtctgtggtgattatgcagcatgtaaatcacagttggtt |
| | | gaatatggtagtttctgtgataacattaatgccatactcacagaagtaaatgaactacttgacactacacagttgcaagtagctaatagtttaatgaatggtgttactcttagcact |
| | | aagcttaaagatggcgttaatttcaatgtagacgacatcaatttttcccctgtattaggttgtctaggcagcgaatgtagtaaagcttccagtagatctgctatagaggatttactt |
| | | tttgataaagtaaagttatctgatgtcggttttgttgaggcttataataattgtacaggaggtgccgaaattagggacctcatttgtgtgcaaagttataaaggcatcaaagtgttg |
| | | cctccactgctctcagaaaatcagatcagtggatacactttggctgccacctctgctagtctatttcctccttggacagcagcagcaggtgtaccattttatttaaatgttcagtat |
| | | cgcattaatgggcttggtgtcaccatggatgtgctaagtcaaaatcaaaagcttattgctaatgcatttaacaatgccctttatgctattcaggaagggttcgatgcaactaattc |
| | | tgattagttaaaattcaagctgttgttaatgcaaatgctgaagctcttaataacttattgcaacaactctctaatagatttggtgctataagtgcttctttacaagaaattctatctag |
| | | acttgatgctcttgaagcggaagctcagatagatagacttattaatggtcgtcttaccgctcttaatgcttatgtttctcaacagcttagtgattctacactggtaaaatttagtgca |
| | | gcacaagctatggagaaggttaatgaatgtgtcaaaagccaatcatctaggataaatttctgtggtaatggtaatcatattatatcattagtgcagaatgctccatatggtttgta |
| | | ttttatccactttagttatgtccctactaagtatgtcacagcgagggttagtcctggtctgtgcattgctggtgatagaggtatagctcctaagagtggttattttgttaatgtaaata |
| | | atacttggatgtacactggtagtggttactactaccctgaacctataactgaaaataatgttgttgttatgagtacctgcgctgttaattatactaaagcgccgtatgtaatgctga |
| | | acacttcaatacccaaccttcctgattttaaggaagagttggatcaatggtttaaaaatcaaacatcagtggcaccagatttgtcacttgattatataaatgttacattcttggacc |
| | | tacaagttgaaatgaataggttacaggaggcaataaaagtcttaaatcagagctacatcaatctcaaggacattggtacatatgaatattatgtaaaatggccttggtatgtatg |
| | | gatttaatctgccttgctggtgtagctatgcttgttttactattcttcatatgctgttgtacaggatgtgggactagttgttttaagaaatgtggtggttgttgtgatgattatactgga |
| | | taccaggagttagtaatcaaaacttcacatgacgactaagttcgtattgattcattgcactgatacttgttagatattttgcaatctagcatttgttaaagttcttaaggccacgc |
| | | cctattaatggacatttggagacctgagaagaaatatctccgttatattaacggttttaatgtctcagaattagaagatgcttgttttaaatttaactatcaatttcctaaagtaggat |
| | | attgtagagttcctagtcatgcttggtgccgtaatcaaggtagattttgtgctacattcactattatggtaaatccaaacattatgataaatattttggagtaataaatggtttcaca |
| | | gcattcgctaatactgtagaggatgctgttaacaaactggttttcttagctgttgactttattacctggcgcagacaggagttaaatgtttatggctgatgcttatcttgcagacac |
| | | tgtgtggtatgtggggcaaataatttttatagttgccatttgtttattggttacaatagttgtagtggcatttttggcaacttttaaattgtgtattcaactttgcggtatgtgtaatacct |
| | | tagtactgtccccttctatttatgtgtttaatagaggtaggcagttttatgagttttacaatgatgtaaaaccaccagtccttgatgtggatgacgtttaggtaatccaaacattatg |
| | | agtagtaaaactacaccagcaccagtttatatctggactgctgatgaagctattaaattcctaaaggaatggaatttttattgggtattatactactttttattacaatcatattgca |
| | | atttggatatacaagtcgcagtatgtttgtttatgttattaagatgattattttgtggcttatgtggccccttactataatcttaactattttcaattgcgtatacgcattgaataatgtgt |
| | | atcttggcctttctatagtttttaccatagtggccattattatgtggattgtgtattttgtgaatagtatcaggttgtttattagaactggaagtttttggagtttcaacccagaaacaaa |
| | | caacttgatgtgtatagatatgaaaggaacaatgtatgttaggccgataattgaggactatcatactctgacggtcacaataatacgcggccatctttacattcaaggtataaaa |
| | | ctaggtactggctattattggcagatttgccagcttatatgactgttgctaaggttacacacctgtgcacatataagcgtggttttcttgacaggataagcgatactagtggtttt |
| | | gctgtttatgttaagtccaaagtcggtaattaccgactgccatcaacccaaaagggttctggcatggacaccgcattgttgagaaataatatctaaattttaaggatgtatttac |
| | | tcctggtaagcaatccagtagtagagcgtcctctggaaatcgttctggtaatggcatcctcaagtgggccgatcagtccgaccagtttagaaatgttcaaaccaggggtaga |
| | | agagctcaacccaagcaaactgctacctctcagcaaccatcaggagggaatgttgtaccctactattcttggttctctggaattactcagtttcaaaagggaaaggagtttga |
| | | gtttgtagaaggacaaggtgtgcctattgcaccaggagtcccagctactgaagctaaggggtactggtacagacacaacagacgttatttaaaacagccgatggcaacc |
| | | agcgtcaactgctgccacgatggtatttttactatctgggaacaggaccgcatgctaaagaccagtacggcaccgatattgacggagtctactgggtcgctagcaaccagg |
| | | ctgatgtcaataccccggctgacattgtcgatcgggacccaagtagcgatgaggctattccgactaggtttccgcctggcacggtactccctcagggttactatattgaagg |
| | | ctcaggaaggtctgctcctaattccagatctacttcgcgcacatccagcagagcctctagtgcaggatcgcgtagtagagccaattctggcaatagaacccctacctctggt |
| | | gtaacacctgacatggctgatcaaattgctagtcttgttctggcaaaacttggcaaggatgccactaaacctcagcaagtaactaagcatactgccaaagaagtcagacaga |
| | | aaattttgaataagccccgccagaagaggagccccaataaacaatgcactgttcagcagtgttttggtaagagaggccctaatcagaattttggtggtggagaaatgttaaa |
| | | acttggaactagtgacccacagttccccattcttgcagaactcgcacccacagctggtgcgtttttattggatcaagattagagttggccaaagtgcagaatttatctgggaat |
| | | cctgacgagccccagaaggatgtttatgaattgcgctataacggcgcaattaggtttgacagtacactttcaggttttgagaccataatgaaggtgctgaatgagaatttgaat |
| | | gcctatcaacaacaagatggtatgatgaatatgagtccaaaaccacagcgtcagcgtggtcataagaatggacaaggagaaaatgataatataagtgttgcagtgcccaa |
| | | aagccgcgtgcagcaaaataagagtagagagttgactgcagaggacatcagccttcttaagaagatggatgagccctatactgaagacacctcagaaatataagagaat |
| | | gaaccttatgtcggcatctggtggtaacccctcgcagaaaagtcgagataaggcactctctatcagaatggatgtcttgctgctataatagatagagaaggttatagcagact |
| | | atagattaattagttgaaagttttgtgttgtaatgtatagtgttggagaaagtgaaagacttgcggaagtaattgccgacaagtgcccaagggaagagccagcatgttaagtta |
| | | ccacccagtaattagtaaatgaatgaagttaattatggccaattggaagaatcacaaaaaaaaaaaaaaaaaaaaaaaaaaaa |

TABLE 4
Biological Assay Results for Assessed siNA

| | SARS-CoV-2 nanoluc | pSiCHECK-2 reporter | pSiCHECK-2 reporter assay Cos-7 at |
|---|---|---|---|
| | hACE-2 A549 assay | assay Cos-7 | least 50% inhibition |
| | A: <0.1 nM EC50; | A: <0.2 nM EC50; | yes (y)/no (n)/undetermimed (u). |
| | B: 0.1-1.0 nM EC50; | B: 0.2-1.0 nM EC50; | siRNAs w/>70% inhib. were followed |
| siNA Name | C: >1 nM EC50 | C: >1 nM EC50 | up w/EC50 (hACE-2 A549 assay) |
| ds-siNA-006 | | | n |
| ds-siNA-007 | | | n |
| ds-siNA-008 | | | n |
| ds-siNA-009 | | | n |
| ds-siNA-010 | | | u |
| ds-siNA-011 | | B | y |
| ds-siNA-012 | | | y |
| ds-siNA-013 | | | n |
| ds-siNA-014 | | | n |
| ds-siNA-015 | | | n |
| ds-siNA-016 | | | n |
| ds-siNA-017 | | | n |
| ds-siNA-018 | | B | y |
| ds-siNA-019 | | | y |
| ds-siNA-020 | | | y |
| ds-siNA-021 | | | y |
| ds-siNA-022 | | | n |
| ds-siNA-023 | | | n |
| ds-siNA-024 | | | n |
| ds-siNA-025 | | | u |
| ds-siNA-026 | | | u |
| ds-siNA-027 | | | y |
| ds-siNA-028 | | | n |
| ds-siNA-029 | | | n |
| ds-siNA-030 | | B | y |
| ds-siNA-031 | | | n |
| ds-siNA-032 | | | n |
| ds-siNA-033 | | | u |
| ds-siNA-034 | | | u |
| ds-siNA-035 | | | n |
| ds-siNA-036 | | | y |
| ds-siNA-037 | | | n |
| ds-siNA-038 | | | n |
| ds-siNA-039 | | A | y |
| ds-siNA-040 | | A | y |
| ds-siNA-041 | | | y |
| ds-siNA-042 | | | n |
| ds-siNA-043 | | | n |
| ds-siNA-044 | | | n |
| ds-siNA-045 | | | n |
| ds-siNA-046 | | | y |
| ds-siNA-047 | | | y |
| ds-siNA-048 | | | y |
| ds-siNA-049 | | | n |
| ds-siNA-050 | | | n |
| ds-siNA-051 | | B | y |
| ds-siNA-052 | | | y |
| ds-siNA-053 | | | n |
| ds-siNA-054 | | | n |
| ds-siNA-055 | | | n |
| ds-siNA-056 | | | n |
| ds-siNA-057 | | | u |
| ds-siNA-058 | | | u |
| ds-siNA-059 | | | u |
| ds-siNA-060 | | | y |
| ds-siNA-061 | | B | y |
| ds-siNA-062 | | B | y |
| ds-siNA-063 | | | n |
| ds-siNA-064 | | | y |
| ds-siNA-065 | | | y |
| ds-siNA-066 | | | n |
| ds-siNA-067 | | B | y |
| ds-siNA-068 | | | n |
| ds-siNA-069 | | | n |
| ds-siNA-070 | | | n |
| ds-siNA-071 | | | n |
| ds-siNA-072 | | | n |
| ds-siNA-073 | | | n |
| ds-siNA-074 | | | n |
| ds-siNA-075 | | C | y |
| ds-siNA-076 | | | y |
| ds-siNA-077 | | | n |
| ds-siNA-078 | | | n |
| ds-siNA-079 | | | u |
| ds-siNA-080 | | | n |
| ds-siNA-081 | | A | y |
| ds-siNA-082 | | B | y |
| ds-siNA-083 | | B | y |
| ds-siNA-084 | | | y |
| ds-siNA-085 | | | y |
| ds-siNA-086 | | | n |
| ds-siNA-087 | | A | y |
| ds-siNA-088 | | | y |
| ds-siNA-089 | | B | y |
| ds-siNA-090 | | | n |
| ds-siNA-091 | | | n |
| ds-siNA-092 | | | n |
| ds-siNA-093 | | | n |
| ds-siNA-094 | | | n |
| ds-siNA-095 | B | B | y |
| ds-siNA-096 | | | y |
| ds-siNA-097 | | | n |
| ds-siNA-098 | | | y |
| ds-siNA-099 | | | n |
| ds-siNA-100 | | | n |
| ds-siNA-101 | | | n |
| ds-siNA-102 | | | n |
| ds-siNA-103 | | | n |
| ds-siNA-104 | | | n |
| ds-siNA-105 | | | u |
| ds-siNA-106 | | B | y |
| ds-siNA-107 | | | y |
| ds-siNA-108 | | | y |
| ds-siNA-109 | | | n |
| ds-siNA-110 | | | n |
| ds-siNA-111 | | | n |
| ds-siNA-112 | | | n |
| ds-siNA-113 | | | y |
| ds-siNA-114 | | | y |
| ds-siNA-115 | | B | y |
| ds-siNA-116 | | | y |
| ds-siNA-117 | | | n |
| ds-siNA-118 | | | n |
| ds-siNA-119 | | | y |
| ds-siNA-120 | | | u |
| ds-siNA-121 | | | u |
| ds-siNA-122 | | | y |
| ds-siNA-123 | | | n |
| ds-siNA-124 | | | n |
| ds-siNA-125 | | B | y |
| ds-siNA-126 | | | n |
| ds-siNA-127 | | | n |
| ds-siNA-128 | | | u |
| ds-siNA-129 | | | u |
| ds-siNA-130 | | | n |
| ds-siNA-131 | | | y |
| ds-siNA-132 | | | n |
| ds-siNA-133 | | | n |
| ds-siNA-134 | | A | y |
| ds-siNA-135 | | A | y |
| ds-siNA-136 | | | y |
| ds-siNA-137 | | | n |
| ds-siNA-138 | | | n |
| ds-siNA-139 | | | n |
| ds-siNA-140 | | | n |
| ds-siNA-141 | | | n |
| ds-siNA-142 | | | y |
| ds-siNA-143 | | | y |
| ds-siNA-144 | | | n |
| ds-siNA-145 | | | n |
| ds-siNA-146 | | B | y |
| ds-siNA-147 | | | n |
| ds-siNA-148 | | | n |
| ds-siNA-149 | | | n |
| ds-siNA-150 | | C | y |
| ds-siNA-151 | | | n |
| ds-siNA-152 | | | u |
| ds-siNA-153 | | | u |
| ds-siNA-154 | | | u |
| ds-siNA-155 | | | y |
| ds-siNA-156 | | B | y |
| ds-siNA-157 | | B | y |
| ds-siNA-158 | | | n |
| ds-siNA-159 | | | y |
| ds-siNA-160 | | | y |
| ds-siNA-161 | | | n |
| ds-siNA-162 | | B | y |
| ds-siNA-163 | | | n |
| ds-siNA-164 | | | y |
| ds-siNA-165 | | | y |
| ds-siNA-166 | | | y |
| ds-siNA-167 | | | y |
| ds-siNA-168 | | | n |
| ds-siNA-169 | | | n |
| ds-siNA-170 | C | B | y |
| ds-siNA-171 | | | y |
| ds-siNA-172 | | | y |
| ds-siNA-173 | | B | y |
| ds-siNA-174 | | | u |
| ds-siNA-175 | | | y |
| ds-siNA-176 | C | A | y |
| ds-siNA-177 | | A | y |
| ds-siNA-178 | | B | y |
| ds-siNA-179 | | B | y |
| ds-siNA-180 | | B | y |
| ds-siNA-181 | | | y |
| ds-siNA-182 | | | y |
| ds-siNA-183 | | A | y |
| ds-siNA-184 | | B | y |
| ds-siNA-185 | | | n |
| ds-siNA-186 | | | n |
| ds-siNA-187 | | | n |
| ds-siNA-188 | | | n |
| ds-siNA-189 | | | n |
| ds-siNA-190 | | | y |
| ds-siNA-191 | | | y |
| ds-siNA-192 | | | y |
| ds-siNA-193 | B | B | y |
| ds-siNA-194 | | | n |
| ds-siNA-195 | | | n |
| ds-siNA-196 | A | B | |
| ds-siNA-197 | B | B | |
| ds-siNA-198 | B | A | |
| ds-siNA-199 | A | B | |
| ds-siNA-217 | | A | |
| ds-siNA-218 | | A | |
| ds-siNA-219 | | A | |
| ds-siNA-220 | | A | |
| ds-siNA-221 | | A | |
| ds-siNA-222 | | A | |

1. A short interfering nucleic acid (siNA) molecule comprising:
(a) a sense strand comprising a first nucleotide sequence, wherein the first nucleotide sequence is 15 to 30 nucleotides in length and comprises a nucleotide sequence that is at least about 60%, 65%, 70%, 75%, 80%, 85%, 90%, 95%, or 100% identical to an RNA corresponding to of any one of SEQ ID NOs: 1-1203 and 2411-3392; and
(b) an antisense strand comprising a second nucleotide sequence, wherein the second nucleotide sequence is 15 to 30 nucleotides in length and comprises a nucleotide sequence that is at least about 60%, 65%, 70%, 75%, 80%, 85%, 90%, 95%, or 100% complementary to the first nucleotide sequence.

2. The siNA molecule of claim 1, wherein the first nucleotide sequence is identical to an RNA corresponding to 15 to 30 nucleotides within positions 190-216, 233-279, 288-324, 455-477, 626-651, 704-723, 3352-3378, 5384-5403, 6406-6483, 7532-7551, 9588-9606, 10484-10509, 11609-11630, 11834-11853, 12023-12045, 12212-12234, 12401-12420, 12839-12867, 12885-12924, 12966-12990, 13151-13176, 13363-13386, 13388-13416, 13458-13416, 13458-13520, 13762-13790, 14290-14312, 14404-14429, 14500-14531, 14623-14642, 14650-14687, 14698-14717, 14722-14748, 14750-14777, 14821-14846, 14854-14873, 14875-14903, 14962-14990, 14992-15020, 15055-15140, 15172-15200, 15310-15332, 15346-15367, 15496-15518, 15622-15644, 15838-15869, 15886-15905, 15985-16010, 16057-16079, 16186-16205, 16430-16448, 16822-16865, 16954-16976, 17008-17042, 17080-17111, 17137-17156, 17269-17289, 17530-17549, 17563-17582, 17680-17699, 17746-17765, 17857-17876, 17956-17975, 18100-18122, 18196-18218, 19618-19639, 19783-19802, 19831-19850, 20107-20130, 20776-20795, 21502-21524, 24302-24325, 24446-24465, 24620-24651, 24662-24684, 25034-25057, 25104-25128, 25364-25387, 25502-25530, 26191-26227, 26232-26267, 26269-26330, 26332-26394, 26450-26481, 26574-26600, 27003-27064, 27093-27111, 27183-27212, 27382-27407, 27511-27533, 27771-27818, 28270-28296, 28397-28434, 28513-28546, 28673-28692, 28706-28726, 28744-28794, 28799-28827, 28946-28972, 28976-29034, 29144-29172, 29174-29196, 29228-29259, 29285-29305, 29342-29394, 29444-29463, 29543-29566, 29598-29630, 29652-29687, 29689-29731, 29733-29757.

3. The siNA molecule of claim 1, wherein the second nucleotide sequence is complementary to an RNA corresponding to 15 to 30 nucleotides within positions 190-216, 233-279, 288-324, 455-477, 626-651, 704-723, 3352-3378, 5384-5403, 6406-6483, 7532-7551, 9588-9606, 10484-10509, 11609-11630, 11834-11853, 12023-12045, 12212-12234, 12401-12420, 12839-12867, 12885-12924, 12966-12990, 13151-13176, 13363-13386, 13388-13416, 13458-13416, 13458-13520, 13762-13790, 14290-14312, 14404-14429, 14500-14531, 14623-14642, 14650-14687, 14698-14717, 14722-14748, 14750-14777, 14821-14846, 14854-14873, 14875-14903, 14962-14990, 14992-15020, 15055-15140, 15172-15200, 15310-15332, 15346-15367, 15496-15518, 15622-15644, 15838-15869, 15886-15905, 15985-16010, 16057-16079, 16186-16205, 16430-16448, 16822-16865, 16954-16976, 17008-17042, 17080-17111, 17137-17156, 17269-17289, 17530-17549, 17563-17582, 17680-17699, 17746-17765, 17857-17876, 17956-17975, 18100-18122, 18196-18218, 19618-19639, 19783-19802, 19831-19850, 20107-20130, 20776-20795, 21502-21524, 24302-24325, 24446-24465, 24620-24651, 24662-24684, 25034-25057, 25104-25128, 25364-25387, 25502-25530, 26191-26227, 26232-26267, 26269-26330, 26332-26394, 26450-26481, 26574-26600, 27003-27064, 27093-27111, 27183-27212, 27382-27407, 27511-27533, 27771-27818, 28270-28296, 28397-28434, 28513-28546, 28673-28692, 28706-28726, 28744-28794, 28799-28827, 28946-28972, 28976-29034, 29144-29172, 29174-29196, 29228-29259, 29285-29305, 29342-29394, 29444-29463, 29543-29566, 29598-29630, 29652-29687, 29689-29731, 29733-29757, or 29770-29828 of SEQ ID NO: 2407.

4. The siNA molecule of claim 1, wherein the sense strand comprises a nucleotide sequence identical to an RNA corresponding to any one of SEQ ID NOs: 1-1203 and 2411-3392.

5. The siNA molecule of claim 1, wherein the antisense strand comprises a nucleotide sequence identical to an RNA corresponding to any one of SEQ ID NOs: 1204-2406 and 3393-4374 and (b) a sense strand.

6. The siNA of claim 1, wherein the sense strand comprises 15 or more modified nucleotides independently selected from a 2′-O-methyl nucleotide and a 2′-fluoro nucleotide, wherein at least one modified nucleotide is a 2′-O-methyl nucleotide and the nucleotide at position 3, 5, 7, 8, 9, 10, 11, 12, 14, 17, and/or 19 from the 5′ end is a 2′-fluoro nucleotide; and the antisense strand comprises 15 or more modified nucleotides independently selected from a 2′-O-methyl nucleotide and a 2′-fluoro nucleotide, wherein at least one modified nucleotide is a 2′-O-methyl nucleotide and at least one modified nucleotide is a 2′-fluoro nucleotide.

7. The siNA of claim 1, wherein the sense strand comprises 15 or more modified nucleotides independently selected from a 2′-O-methyl nucleotide and a 2′-fluoro nucleotide, wherein at least one modified nucleotide is a 2′-O-methyl nucleotide and at least one modified nucleotide is a 2′-fluoro nucleotide; and the antisense strand comprises 15 or more modified nucleotides independently selected from a 2′-O-methyl nucleotide and a 2′-fluoro nucleotide, wherein at least one modified nucleotide is a 2′-O-methyl nucleotide and the nucleotide at position 2, 5, 6, 8, 10, 14, 16, 17, and/or 18 from the 5′ end is a 2′-fluoro nucleotide.

8. The siNA of claim 1, wherein the sense strand comprises 16, 17, 18, 19, 20, 21, 22, 23, or more modified nucleotides independently selected from a 2′-O-methyl nucleotide and a 2′-fluoro nucleotide.

9. The siNA of claim 1, wherein 70%, 75%, 80%, 85%, 90%, 95% or 100% of the nucleotides in the sense strand are modified nucleotides independently selected from a 2′-O-methyl nucleotide and a 2′-fluoro nucleotide.

10. The siNA of claim 1, wherein:
(i) at least 2, 3, 4, 5, or 6 modified nucleotides of the sense strand are 2′-fluoro nucleotides;
(ii) no more than 10, 9, 8, 7, 6, 5, 4, 3, or 2 modified nucleotides of the sense strand are 2′-fluoro nucleotides;
(iii) at least 10, 11, 12, 13, 14, 15, 16, 17, 18, 19, 20, 21, or 22 modified nucleotides of the sense strand sequence are 2′-O-methyl nucleotides; and/or
(iv) no more than 25, 24, 23, 22, 21, 20, 19, 18, 17, 16, 15, 14, 13, 12, 11, 10, 9, 8, 7, 6, 5, 4, 3, or 2 modified nucleotides of the sense strand are 2′-O-methyl nucleotides.

11. The siRNA of claim 1, wherein the antisense strand comprises 16, 17, 18, 19, 20, 21, 22, 23, or more modified nucleotides independently selected from a 2′-O-methyl nucleotide and a 2′-fluoro nucleotide.

12. The siNA of claim 1, wherein 70%, 75%, 80%, 85%, 90%, 95% or 100% of the nucleotides in the antisense strand are modified nucleotides independently selected from a 2′-O-methyl nucleotide and a 2′-fluoro nucleotide.

13. The siNA of claim 1, wherein:
(i) at least 2, 3, 4, 5, or 6 modified nucleotides of the antisense strand are 2′-fluoro nucleotides;
(ii) no more than 10, 9, 8, 7, 6, 5, 4, 3, or 2 modified nucleotides of the antisense strand are 2′-fluoro nucleotides;
(iii) at least 10, 11, 12, 13, 14, 15, 16, 17, 18, 19, 20, 21, or 22 modified nucleotides of the antisense strand sequence are 2′-O-methyl nucleotides; and/or
(iv) no more than 25, 24, 23, 22, 21, 20, 19, 18, 17, 16, 15, 14, 13, 12, 11, 10, 9, 8, 7, 6, 5, 4, 3, or 2 modified nucleotides of the antisense strand are 2′-O-methyl nucleotides.

14. The siNA of claim 1, wherein the sense strand and/or the antisense strand comprise one or more phosphorothioate internucleoside linkage(s).

15. The siNA of claim 1, wherein the siNA further comprises a phosphorylation blocker and/or a 5′-stabilized end cap.

16. The siNA of claim 1, wherein the sense strand further comprises a TT sequence adjacent to the first nucleotide sequence.

17. The siNA of claim 1, wherein at least one end of the siNA is a blunt end.

18. The siNA of claim 1, wherein at least one end of the siNA comprises an overhang, wherein the overhang comprises at least one nucleotide.

19. The siNA of claim 1, wherein at both ends of the siNA comprise an overhang, wherein the overhang comprises at least one nucleotide.

20. The siNA of claim 1, wherein the sense strand and/or the antisense strand comprises one or more modified nucleotides.

21. The siNA of claim 1, wherein the sense strand and/or the antisense strand further comprises at least 1, 2, 3, 4, 5, 6, 7, 8, 9, 10, 11, 12, 13, 14, 15 or more phosphorothioate internucleotide linkages.

22-23. (canceled)

24. The siNA of claim 20, wherein the modified nucleotides are independently selected from 2′-O-methyl nucleotides and 2′-fluoro nucleotides.

25. The siNA of claim 0, wherein at least one 2′-fluoro nucleotide or 2′-O-methyl nucleotide is a 2′-fluoro or 2′-O-methyl nucleotide mimic of Formula (V): 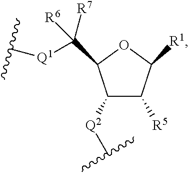
wherein
R¹ is a nucleobase, aryl, heteroaryl, or H,
Q¹ and Q² are independently S or O,
R⁵ is —OCD₃, —F, or —OCH₃, and
R⁶ and R⁷ are independently H or D;
or a 2′-fluoro nucleotide 2′-fluoro nucleotide mimic selected from

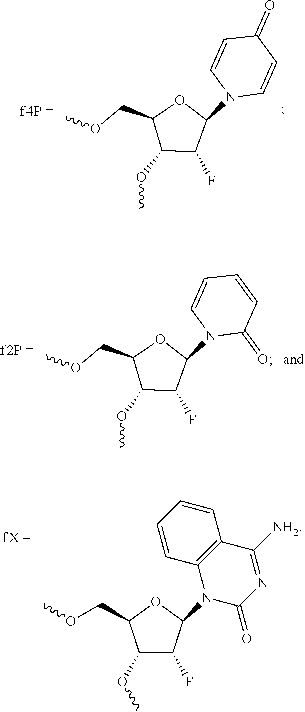

26. The siNA of claim 1, wherein the sense strand and/or antisense strand comprises at least one modified nucleotide selected from 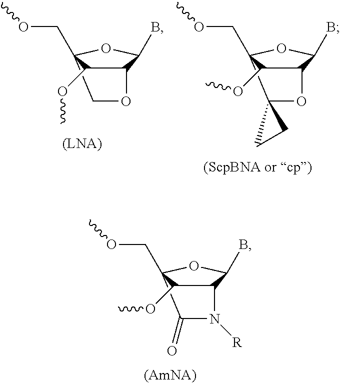
where R is H or alkyl (or AmNA(N-Me)) when R is alkyl); 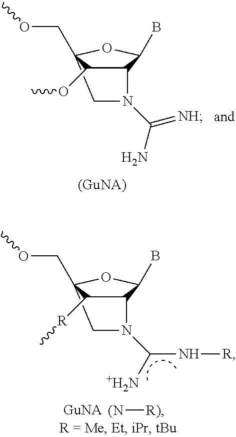
wherein B is a nucleobase.

27. The siNA of claim 1, wherein the ds-siNA further comprises a phosphorylation blocker and/or a 5′-stabilized end cap.

28. The siNA of claim 0, wherein the phosphorylation blocker has the structure of Formula (IV): 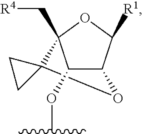
wherein
R¹ is a nucleobase,
R⁴ is —O—R³⁰ or —NR³¹R³²,
R³⁰ is C₁-C₈ substituted or unsubstituted alkyl; and
R³¹ and R³² together with the nitrogen to which they are attached form a substituted or unsubstituted heterocyclic ring.

29. The siNA of claim 0, wherein R⁴ is —OCH₃ or —N(CH₂CH₂)₂O.

30. The siNA of claim 26, wherein the phosphorylation blocker is attached to the 5′ end of the sense strand.

31. The siNA of claim 0, wherein the phosphorylation blocker is attached to the 5′ end of the sense strand via one or more linkers independently selected from a phosphodiester linker, phosphorothioate linker, and phosphorodithioate linker.

32. The siNA of claim 0, wherein the 5′-stabilized end cap is a 5′ vinylphosphonate.

33. The siNA of claim 32, wherein the 5′ vinylphosphonate is selected from a 5′-(E)-vinyl phosphonate or 5′-(Z)-vinyl phosphonate.

34. The siNA of claim 32, wherein the 5′-vinylphosphonate is a deuterated vinyl phosphonate.

35. The siNA of claim 34, wherein the deuterated vinylphosphonate is a mono-deuterated vinylphosphonate or a di-deuterated vinylphosphonate.

36. The siNA of claim 0, wherein the 5′-stabilized end cap has the structure of Formula (Ia): 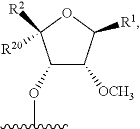
wherein
R¹ is a nucleobase, aryl, heteroaryl, or H,
R² is 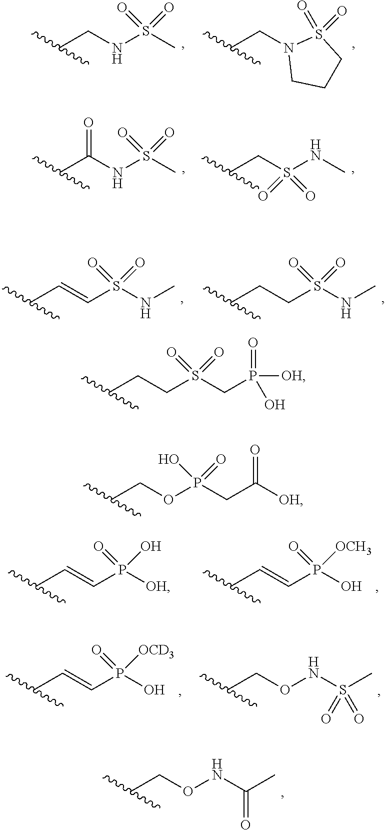
—CH═CD-Z, —CD=CH—Z, —CD=CD-Z, —(CR²¹R²²)ₙ—Z, or —(C₂-C₆ alkenylene)-Z and R²⁰ is hydrogen; or
R² and R²⁰ together form a 3- to 7-membered carbocyclic ring substituted with —(CR²¹R²²)ₙ—Z or —(C₂-C₆ alkenylene)-Z;
n is 1, 2, 3, or 4;
Z is —ONR²³R²⁴, —OP(O)OH(CH₂)ₘCO₂R²³, —OP(S)OH(CH₂)ₘCO₂R²³, —P(O)(OH)₂, —P(O)(OH)(OCH₃), —P(O)(OH)(OCD₃), —SO₂(CH₂)ₘP(O)(OH)₂, —SO₂NR²³R²⁵, —NR²³R²⁴, or —NR²³SO₂R²⁵;
R²¹ and R²² either are independently hydrogen or C₁-C₆ alkyl, or R²¹ and R²² together form an oxo group;
R²³ is hydrogen or C₁-C₆ alkyl;
R²⁴ is —SO₂R²⁵ or —C(O)R²⁵; or
R²³ and R²⁴ together with the nitrogen to which they are attached form a substituted or unsubstituted heterocyclic ring;
R²⁵ is C₁-C₆ alkyl; and
m is 1, 2, 3, or 4.

37. The siNA of claim 0, wherein R¹ is an aryl.

38. The siNA of claim 0, wherein the aryl is a phenyl.

39. The siNA of claim 26, wherein the 5′-stabilized end cap has the structure of Formula (Ib): 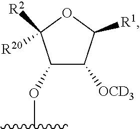
wherein
R¹ is a nucleobase, aryl, heteroaryl, or H,
R² is 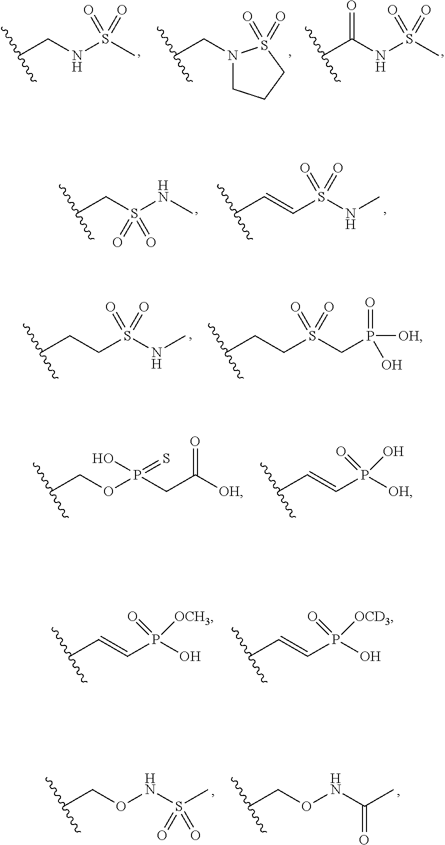
—CH═CD-Z, —CD=CH—Z, —CD=CD-Z, —(CR²¹R²²)ₙ—Z, or —(C₂-C₆ alkenylene)-Z and R²⁰ is hydrogen; or
R² and R²⁰ together form a 3- to 7-membered carbocyclic ring substituted with —(CR²¹R²²)ₙ—Z or —(C₂-C₆ alkenylene)-Z;
n is 1, 2, 3, or 4;
Z is —ONR²³R²⁴, —OP(O)OH(CH₂)ₘCO₂R²³, —OP(S)OH(CH₂)ₘCO₂R²³, —P(O)(OH)₂, —P(O)(OH)(OCH₃), —P(O)(OH)(OCD₃), —SO₂(CH₂)ₘP(O)(OH)₂, —SO₂NR²³R²⁵, —NR²³R²⁴,
R²¹ and R²² are independently hydrogen or C₁-C₆ alkyl; R²¹ and R²² together form an oxo group;
R²³ is hydrogen or C₁-C₆ alkyl;
R²⁴ is —SO₂R²⁵ or —C(O)R²⁵; or
R²³ and R²⁴ together with the nitrogen to which they are attached form a substituted or unsubstituted heterocyclic ring;
R²⁵ is C₁-C₆ alkyl; and
m is 1, 2, 3, or 4.

40. The siNA of claim 26, wherein the 5′-stabilized end cap is selected from the group consisting of Formula (1) to Formula (15), Formula (9X) to Formula (12X), and Formula (9Y) to Formula (12Y): 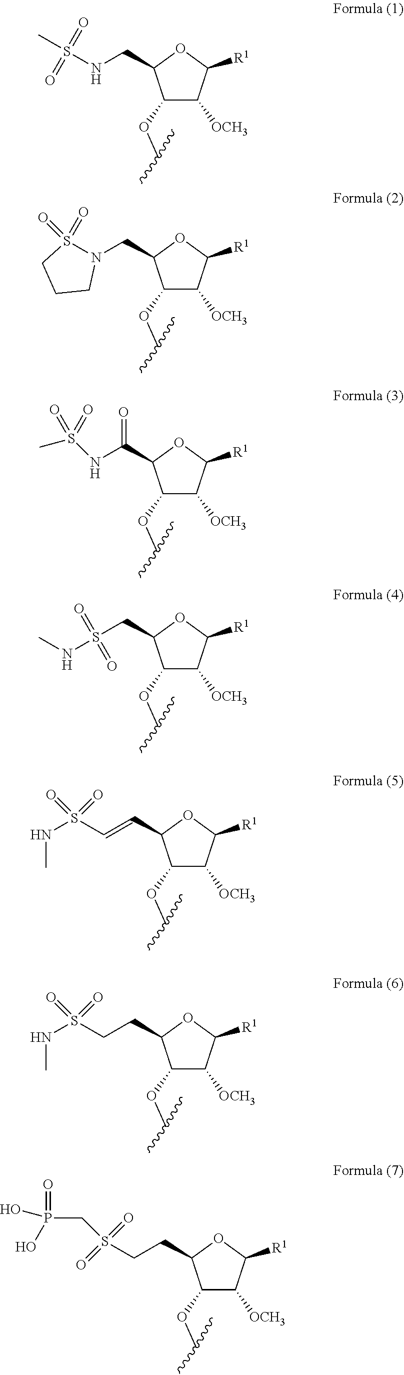 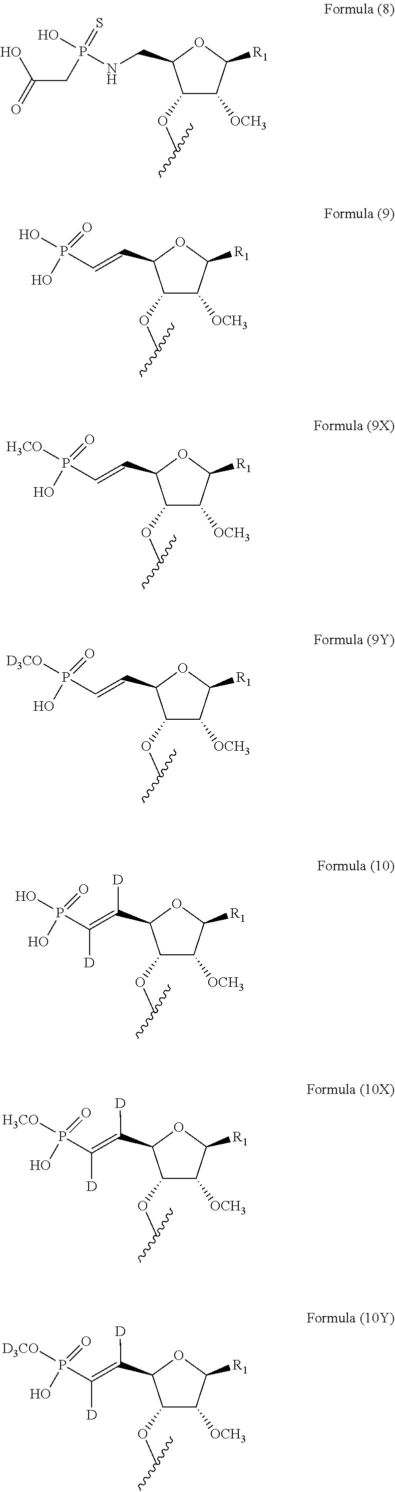 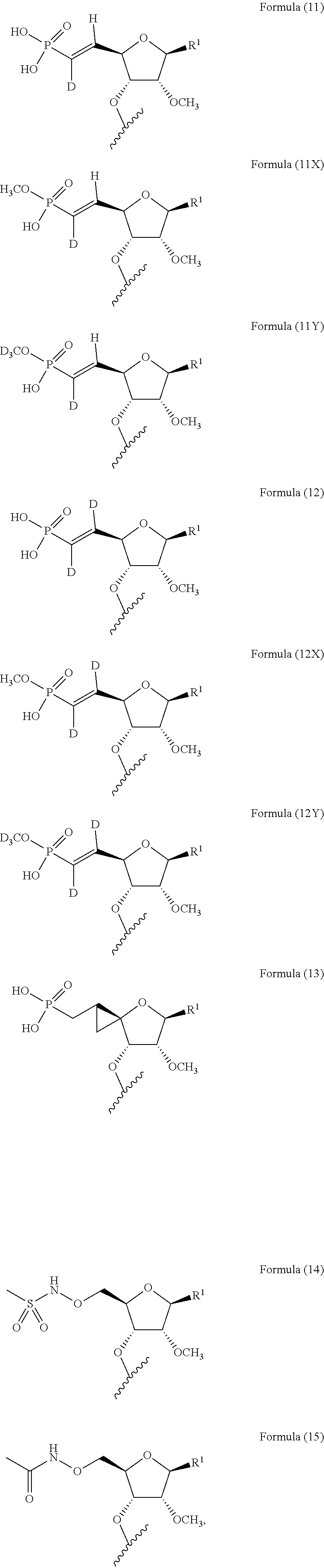
wherein R¹ is a nucleobase, aryl, heteroaryl, or H.

41. The siNA of claim 26, wherein the 5′-stabilized end cap is selected from the group consisting of Formulas (1A)-(15A), Formulas (9B)-(12B), Formulas (9AX)-(12AX), Formulas (9AY)-(12AY), Formulas (9BX)-(12BX), and Formulas (9BY)-(12BY):

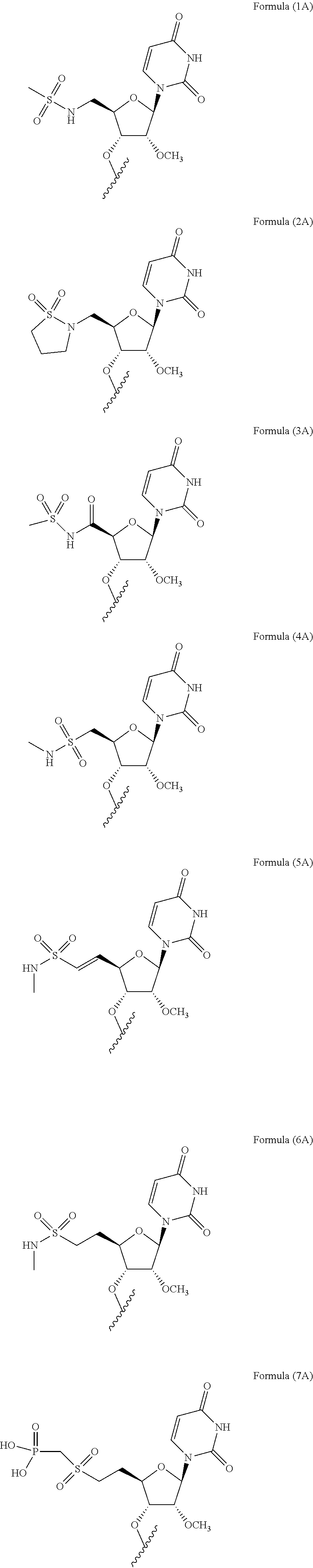

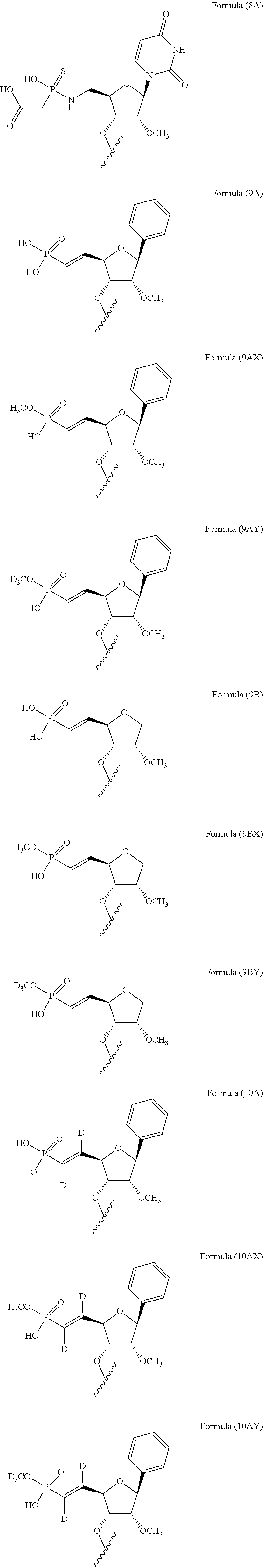

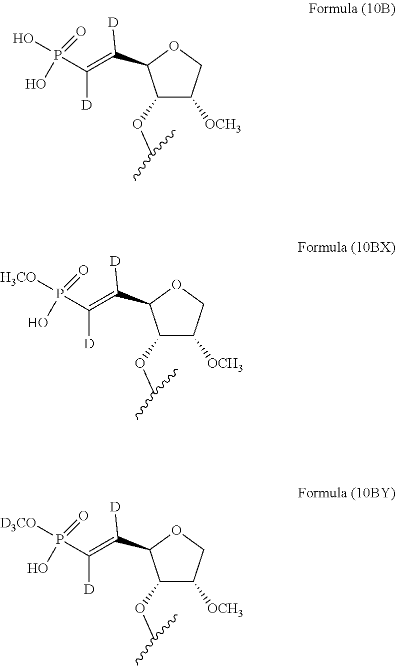

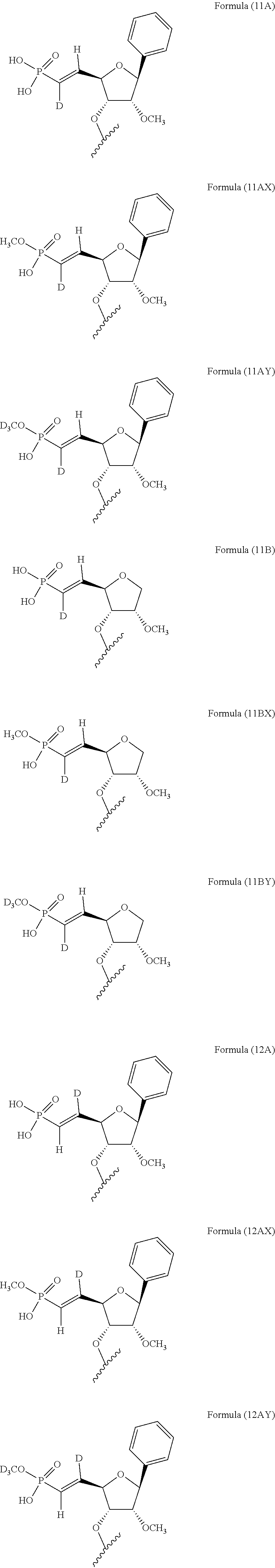

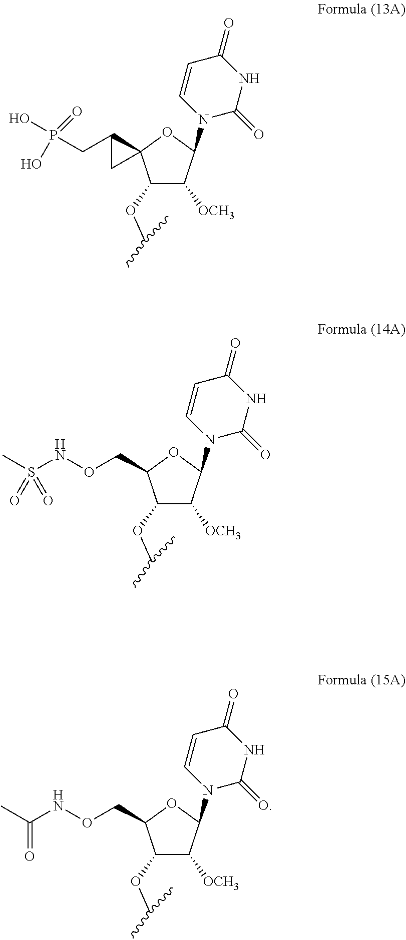

42. The siNA of any of claim 26, wherein the 5′-stabilized end cap is attached to the 5′ end of the antisense strand.

43. The siNA of claim 42, wherein the 5′-stabilized end cap is attached to the 5′ end of the antisense strand via one or more linkers independently selected from a phosphodiester linker, phosphorothioate linker, phosphoramidite (HEG) linker, triethylene glycol (TEG) linker, or phosphorodithioate linker.

44. The siNA molecule of claim 1, wherein the sense strand consists of 21 nucleotides.

45. The siNA molecule of claim 44, wherein 2′-O-methyl nucleotides are at positions 18-21 from the 5′ end of the sense strand.

46. The siNA molecule of claim 1, wherein the antisense strand consists of 23 nucleotides.

47. The siNA molecule of claim 46, wherein 2′-O-methyl nucleotides are at positions 18-23 from the 5′ end of the antisense strand.

48. An siNA selected from ds-siNA-005; ds-siNA-006; ds-siNA-007; ds-siNA-008; ds-siNA-009; ds-siNA-010; ds-siNA-011; ds-siNA-012; ds-siNA-013; ds-siNA-014; ds-siNA-015; ds-siNA-016; ds-siNA-017; ds-siNA-018; ds-siNA-019; ds-siNA-020; ds-siNA-021; ds-siNA-022; ds-siNA-023; ds-siNA-024; ds-siNA-025; ds-siNA-026; ds-siNA-027; ds-siNA-028; ds-siNA-029; ds-siNA-030; ds-siNA-031; ds-siNA-032; ds-siNA-033; ds-siNA-034; ds-siNA-035; ds-siNA-036; ds-siNA-037; ds-siNA-038; ds-siNA-039; ds-siNA-040; ds-siNA-041; ds-siNA-042; ds-siNA-043; ds-siNA-044; ds-siNA-045; ds-siNA-046; ds-siNA-047; ds-siNA-048; ds-siNA-049; ds-siNA-050; ds-siNA-051; ds-siNA-052; ds-siNA-053; ds-siNA-054; ds-siNA-055; ds-siNA-056; ds-siNA-057; ds-siNA-058; ds-siNA-059; ds-siNA-060; ds-siNA-061; ds-siNA-062; ds-siNA-063; ds-siNA-064; ds-siNA-065; ds-siNA-066; ds-siNA-067; ds-siNA-068; ds-siNA-069; ds-siNA-070; ds-siNA-071; ds-siNA-072; ds-siNA-073; ds-siNA-074; ds-siNA-075; ds-siNA-076; ds-siNA-077; ds-siNA-078; ds-siNA-079; ds-siNA-080; ds-siNA-081; ds-siNA-082; ds-siNA-083; ds-siNA-084; ds-siNA-085; ds-siNA-086; ds-siNA-087; ds-siNA-088; ds-siNA-089; ds-siNA-090; ds-siNA-091; ds-siNA-092; ds-siNA-093; ds-siNA-094; ds-siNA-095; ds-siNA-096; ds-siNA-097; ds-siNA-098; ds-siNA-099; ds-siNA-100; ds-siNA-101; ds-siNA-102; ds-siNA-103; ds-siNA-104; ds-siNA-105; ds-siNA-106; ds-siNA-107; ds-siNA-108; ds-siNA-109; ds-siNA-110; ds-siNA-111; ds-siNA-112; ds-siNA-113; ds-siNA-114; ds-siNA-115; ds-siNA-116; ds-siNA-117; ds-siNA-118; ds-siNA-119; ds-siNA-120; ds-siNA-121; ds-siNA-122; ds-siNA-123; ds-siNA-124; ds-siNA-125; ds-siNA-126; ds-siNA-127; ds-siNA-128; ds-siNA-129; ds-siNA-130; ds-siNA-131; ds-siNA-132; ds-siNA-133; ds-siNA-134; ds-siNA-135; ds-siNA-136; ds-siNA-137; ds-siNA-138; ds-siNA-139; ds-siNA-140; ds-siNA-141; ds-siNA-142; ds-siNA-143; ds-siNA-144; ds-siNA-145; ds-siNA-146; ds-siNA-147; ds-siNA-148; ds-siNA-149; ds-siNA-150; ds-siNA-151; ds-siNA-152; ds-siNA-153; ds-siNA-154; ds-siNA-155; ds-siNA-156; ds-siNA-157; ds-siNA-158; ds-siNA-159; ds-siNA-160; ds-siNA-161; ds-siNA-162; ds-siNA-163; ds-siNA-164; ds-siNA-165; ds-siNA-166; ds-siNA-167; ds-siNA-168; ds-siNA-169; ds-siNA-170; ds-siNA-171; ds-siNA-172; ds-siNA-173; ds-siNA-174; ds-siNA-175; ds-siNA-176; ds-siNA-177; ds-siNA-178; ds-siNA-179; ds-siNA-180; ds-siNA-181; ds-siNA-182; ds-siNA-183; ds-siNA-184; ds-siNA-185; ds-siNA-186; ds-siNA-187; ds-siNA-188; ds-siNA-189; ds-siNA-190; ds-siNA-191; ds-siNA-192; ds-siNA-193; ds-siNA-194; ds-siNA-195; ds-siNA-196; ds-siNA-197; ds-siNA-198; ds-siNA-199; ds-siNA-200; ds-siNA-201; ds-siNA-202; ds-siNA-203; ds-siNA-204; ds-siNA-205; ds-siNA-206; ds-siNA-207; ds-siNA-208; ds-siNA-209; ds-siNA-210; ds-siNA-211; ds-siNA-212; ds-siNA-213; ds-siNA-214; ds-siNA-215; ds-siNA-216; ds-siNA-217; ds-siNA-218; ds-siNA-219; ds-siNA-220; ds-siNA-221; and ds-siNA-222.

49. The siNA of claim 48, wherein the siNA is selected from ds-siNA-196 (sense and antisense respectively comprising SEQ ID NOs: 4578 and 4800), ds-siNA-197 (sense and antisense respectively comprising SEQ ID NOs: 4579 and 4801), ds-siNA-198 (sense and antisense respectively comprising SEQ ID NOs: 4580 and 4802), ds-siNA-199 (sense and antisense respectively comprising SEQ ID NOs: 4581 and 4803), ds-siNA-217 (sense and antisense respectively comprising SEQ ID NOs: 4599 and 4821), ds-siNA-218 (sense and antisense respectively comprising SEQ ID NOs: 4600 and 4822), ds-siNA-219 (sense and antisense respectively comprising SEQ ID NOs: 4601 and 4823), ds-siNA-220 (sense and antisense respectively comprising SEQ ID NOs: 4602 and 4824), ds-siNA-221 (sense and antisense respectively comprising SEQ ID NOs: 4603 and 4825), and ds-siNA-222 (sense and antisense respectively comprising SEQ ID NOs: 4604 and 4826).

50. The siNA of claim 48, wherein the siNA is selected from ds-siNA-196 (sense and antisense respectively comprising SEQ ID NOs: 4578 and 4800), ds-siNA-197 (sense and antisense respectively comprising SEQ ID NOs: 4579 and 4801), ds-siNA-198 (sense and antisense respectively comprising SEQ ID NOs: 4580 and 4802), and ds-siNA-199 (sense and antisense respectively comprising SEQ ID NOs: 4581 and 4803).

51. The siNA of claim 48, wherein the siNA is selected from, ds-siNA-217 (sense and antisense respectively comprising SEQ ID NOs: 4599 and 4821), ds-siNA-218 (sense and antisense respectively comprising SEQ ID NOs: 4600 and 4822), ds-siNA-219 (sense and antisense respectively comprising SEQ ID NOs: 4601 and 4823), ds-siNA-220 (sense and antisense respectively comprising SEQ ID NOs: 4602 and 4824), ds-siNA-221 (sense and antisense respectively comprising SEQ ID NOs: 4603 and 4825), and ds-siNA-222 (sense and antisense respectively comprising SEQ ID NOs: 4604 and 4826).

52. A pharmaceutical composition comprising at least one siNA according to claim 1 and a pharmaceutically acceptable carrier or diluent.

53. A pharmaceutical composition comprising at least one siNA according to claim 48 and a pharmaceutically acceptable carrier or diluent.

54-55. (canceled)

56. A method of treating a disease caused by a β-coronavirus in a subject in need thereof, comprising administering the subject at least one siNA according to claim 1.

57-75. (canceled)

76. A method of treating a β-coronavirus-caused disease in a subject in need thereof, comprising administering the subject at least one siNA of claim 48.

77-83. (canceled)
